(19) United States
(12) Patent Application Publication
(10) Pub. No.: US 2021/0328150 A1
(43) Pub. Date: Oct. 21, 2021

(54) ORGANIC ELECTROLUMINESCENT COMPOUND, ORGANIC ELECTROLUMINESCENT MATERIAL COMPRISING THE SAME, AND ORGANIC ELECTROLUMINESCENT DEVICE

(71) Applicant: ROHM AND HAAS ELECTRONIC MATERIALS KOREA LTD., Chungcheongnam-do (KR)

(72) Inventors: Hyo-Soon PARK, Gyeonggi-do (KR); Doo-Hyeon Moon, Gyeonggi-do (KR); Du-Yong Park, Gyeonggi-do (KR); Hong-Se Oh, Gyeonggi-do (KR); Kyoung-Jin Park, Gyeonggi-do (KR); Dong-Hyung Lee, Gyeonggi-do (KR); Sang-Hee Cho, Gyeonggi-do (KR); So-Mi Park, Gyeonggi-do (KR); Jin-Man Kim, Gyeonggi-do (KR)

(21) Appl. No.: 17/206,835

(22) Filed: Mar. 19, 2021

(30) Foreign Application Priority Data
Mar. 20, 2020 (KR) 10-2020-0034422
Nov. 6, 2020 (KR) 10-2020-0147603
Feb. 19, 2021 (KR) 10-2021-0022715

(51) Int. Cl. H01L 51/00

(52) U.S. Cl. CPC H01L 51/0067; H01L 51/0061; H01L 51/006; H01L 51/5016; H01L 51/0072; H01L 51/0058; H01L 2251/5384; H01L 51/0073

(57) ABSTRACT

The present disclosure relates to an organic electroluminescent compound, an organic electroluminescent material comprising the same, and an organic electroluminescent device. By comprising an organic electroluminescent compound according to the present disclosure and an organic electroluminescent material comprising the same, an organic electroluminescent device having low driving voltage and/or high luminous efficiency and/or long lifespan can be provided.

### TECHNICAL FIELD

The present disclosure relates to an organic electroluminescent compound, an organic electroluminescent material comprising the same, and an organic electroluminescent device.

### BACKGROUND ART

Among a display device, an electroluminescent device (EL device) is a self-light-emitting display device which has advantages in that it provides a wider viewing angle, a greater contrast ratio, and a faster response time. An organic EL device was first developed by Eastman Kodak in 1987, by using small aromatic diamine molecules and aluminum complexes as materials for forming a light-emitting layer [Appl. Phys. Lett. 51, 913, 1987].

An organic electroluminescent device (OLED) consists of a multi-layer structure including a hole injection layer, a hole transport layer, a light-emitting layer, an electron transport layer, and an electron injection layer, etc., in order to improve its efficiency and stability. In this case, the selection of a compound included in the hole transport layer or the like is recognized as one of the means for improving device properties such as the hole transport efficiency to a light-emitting layer, the luminous efficiency, and the lifespan.

In this regard, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc., were used as a compound comprised in a hole injection and transport material in an OLED. However, an OLED prepared using these materials have problems of reduction in quantum efficiency and lifespan. This is due to circumstances when an OLED is driven under high current, thermal stress occurs between an anode and a hole injection layer, thereby such thermal stress significantly reduces the lifespan of the device. Further, since the organic material used in the hole injection layer has very high hole mobility, there have been problems in that the hole-electron charge balance is broken and the quantum efficiency (cd/A) is lowered.

Therefore, the development of a material for a hole transport layer for improving the performance of an OLED is still required.

U.S. Pat. No. 8,343,637 B2 discloses a compound in which tetramethylphenanthrene is used as a linker of a carbazole-carbazole compound as an example of a host material. However, said reference does not disclose specific device Examples, and the synthesis methods for said compound. In addition, said compound in the reference is not used as a material for a hole transport layer.

### DISCLOSURE OF THE INVENTION

### Technical Problem

The object of the present disclosure is firstly, to provide an organic electroluminescent compound which can be prepared for an organic electroluminescent device having low driving voltage and/or high luminous efficiency and/or long lifespan an organic electroluminescent compound, and secondly, to provide an organic electroluminescent device comprising the organic electroluminescent compound.

### Solution to Problem

As a result of intensive studies to solve the technical problem above, the present inventors found that a compound represented by the following formula 1 which has a dihydrophenanthrene moiety, exhibits improved deterioration properties, so that the present invention was completed.

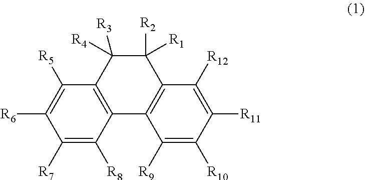

In formula 1,

R₁ to R₄ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent(s) to form a ring(s);

R₅ to R₁₂ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of an (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s);

provided that at least one of R₁ to R₁₂ represent(s) *-(L₁)ₐ(Ar₁)_{b};

L₁ represents a single bond, a substituted or unsubstituted (C6-C30)arylene, or a substituted or unsubstituted (3- to 30-membered)heteroarylene;

Ar₁ represents a substituted or unsubstituted (C6-C30)aryl, a substituted or unsubstituted (3- to 30-membered)heteroaryl, or —N—(Ar₂)(Ar₃);

Ar₂ and Ar₃ each independently represent a substituted or unsubstituted (C1-C30)alkyl, a substituted or unsubstituted (C2-C30)alkenyl, a substituted or unsubstituted fused ring of an (C3-C30) aliphatic ring and a (C6-C30) aromatic ring, a substituted or unsubstituted (C6-C30)aryl, or a substituted or unsubstituted (3- to 30-membered)heteroaryl; and

a represents an integer of 1 or 2, and b represents an integer of 1 to 4; and when a and b are 2 or more, each of L₁ and each of Ar₁ may be the same or different;

provided that the compounds of formula 1 where R₅ to R₁₀ and R₁₂ are hydrogen, and R₁₁ includes a substituted amino group, are excluded.

### Advantageous Effects of Invention

An organic electroluminescent device having low driving voltage and/or high luminous efficiency and/or long lifespan can be manufactured by comprising an organic electroluminescent compound according to the present disclosure and an organic electroluminescent material comprising the same.

### MODE FOR THE INVENTION

Hereinafter, the present disclosure will be described in detail. However, the following description is intended to explain the invention, and is not meant in any way to restrict the scope of the invention.

The present disclosure relates to an organic electroluminescent compound represented by formula 1 above, an organic electroluminescent material comprising the organic electroluminescent compound, and an organic electroluminescent device comprising the organic electroluminescent material.

Further, the present disclosure relates to an organic electroluminescent compound represented by formula 2 and an organic electroluminescent device comprising the organic electroluminescent compound.

Furthermore, the present disclosure relates to an organic electroluminescent compound represented by formula 3 and an organic electroluminescent device comprising the organic electroluminescent compound.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including host and dopant materials), an electron buffer material, a hole blocking material, an electron transport material, or an electron injection material, etc.

The term “a plurality of host materials” in the present disclosure means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The two or more compounds comprised in the plurality of host materials of the present disclosure may be included in one light-emitting layer or may be respectively included in different light-emitting layers. When the at least two host materials are comprised in one layer, the at least two host materials may be mixture-evaporated to form a layer, or simultaneously may be co-evaporated individually to form a layer.

The term “(C1-C30)alkyl” in the present disclosure is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C3-C30)cycloalkyl” in the present disclosure is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms, preferably 5 to 7 ring backbone atoms and at least one heteroatoms selected from the group consisting of B, N, O, S, Si. and P, preferably 0, S, and N, and includes tetrahydrofuran, pyrrolidine, thiolan, tetrahydropyran, etc. The term “(C6-C30)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenyifluorenyl, dimethylfluorenyl, diphenyifluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The term “(3- to 30-membered)heteroaryl(ene)” in the present disclosure is an aryl having 3 to 30 ring backbone atoms including at least one heteroatoms selected from the group consisting of B, N, O, S, Si, P, Se, and Ge, preferably at least one heteroatoms selected from N, O, and S in which the number of the ring backbone carbon atoms is preferably 5 to 25. The number of the heteroatoms in the heteroaryl is preferably 1 to 4. The above heteroaryl may be a monocyclic ring, or a fused ring condensed with at least one benzene ring; and may be partially saturated. Also, the above heteroaryl herein may be one formed by linking at least one heteroaryl or aryl group to a heteroaryl group via a single bond(s). Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotrazolyl, phenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyrdinyl, 3-imidazopyridinyl, 5-imidazopyridinyl, 6-imidazopyridinyl, 7-imidazopyridinyl, 8-imidazopyrdinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthrdinyl, 7-phenanthridinyl, 8-phenanthridinyl, 9-phenanthrdinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring of an (C3-C30) aliphatic ring and an (C6-C30) aromatic ring” in the present disclosure means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of an (C3-C30) aliphatic ring and an (C6-C30) aromatic ring may be replaced with at least one heteroatoms selected from B, N, O, S, Si and P, preferably at least one heteroatoms selected from N, O and S. The term “halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, i.e., at the 1 and 2 positions on benzene. Meta position is the next substitution position of the immediately adjacent substitution position, i.e., a compound with substituents at the 1 and 3 positions on benzene. Para position is the next substitution position of the meta position, i.e., a compound with substituents at the 1 and 4 positions on benzene.

The term “a ring formed in linking to an adjacent substituent” in the present disclosure means a substituted or unsubstituted (3- to 30-membered) mono- or polycyclic, alicyclic, aromatic ring, or a combination thereof, preferably a substituted or unsubstituted (3- to 26-membered) mono- or polycyclic, alicyclic, aromatic ring, or a combination thereof, formed by linking or fusing two or more adjacent substituents, Further, the formed ring may be included at least one heteroatoms selected from the group consisting of B, N, O, S, Si and P, preferably at least one heteroatoms selected from the group consisting of N, O and S. According to one embodiment of the present disclosure, the number of atoms in the ring skeleton is 5 to 20; according to another embodiment of the present disclosure, the number of atoms in the ring skeleton is 5 to 15. The linked or fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, the term “substituted” in the expression “substituted or unsubstituted” means that a hydrogen atom in a certain functional group is replaced with another atom or functional group, i.e., a substituent. Preferably, the substituent of the substituted (C1-C30)alkyl, the substituted (C2-C30)alkenyl, the substituted (C6-C30)aryl(ene), the substituted (3- to 30-membered)heteroaryl(ene), the substituted (C3-C30)cycloalkyl, the substituted (3- to 7-membered)heterocycloalkyl, the substituted fused ring of (C3-C30) aliphatic ring and (C6-C30) aromatic ring, the substituted tri(C1-C30)alkylsilyl, the substituted di(C1-C30)alkyl(C6-C30)arylsilyl, the substituted (C1-C30)alkyldi(C6-C30)arylsilyl, and the substituted tri(C6-C30)arylsilyl in the present disclosure, each independently represents at least one selected from the group consisting of deuterium, halogen, cyano, carboxyl, nitro, hydroxy, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (5- to 30-membered)heteroaryl unsubstituted or substituted with (C6-C30)aryl, (C6-C30)aryl unsubstituted or substituted with (5- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, a fused ring of an (C3-C30) aliphatic ring and an (C6-C30) aromatic ring, amino, mono- or di-(C1-C30)alkylamino, mono- or di-(C2-C30)alkenylamino, (C1-C30)alkyl(C2-C30)alkenylamino, a substituted or unsubstituted mono- or di-(C6-C30)arylamino, (C1-C30)alkyl(C6-C30)arylamino, mono- or di-(3- to 30-membered)heteroarylamino, (C1-C30)alkyl(3- to 30-membered)heteroarylamino, (C2-C30)alkenyl(C6-C30)arylamino, (C2-C30)alkenyl(3- to 30-membered)heteroarylamino, (C6-C30)aryl(3- to 30-membered)heteroarylamino, (C1-C30)alkylcarbonyl. (C1-C30)alkoxycarbonyl, (C6-C30)arylcarbonyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl. For example, the substituent may be methyl, phenyl, naphthyl, p-biphenyl, m-biphenyl, m-terphenyl, fluorenyl, phenanthrenyl, pyridyl, dibenzothiophenyl, or dibenzofuranyl, etc.

Hereinafter, the organic electroluminescent compound according to one embodiment will be described.

The organic electroluminescent compound according to one embodiment is represented by the following formula 1.

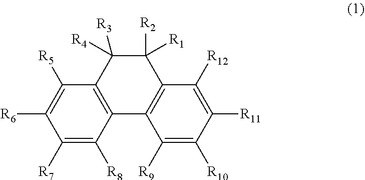

In formula 1,

R₁ to R₄ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent(s) to form a ring(s);

R₅ to R₁₂ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of an (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s):

provided that at least one of R₁ to R₁₂ represents(s) *-(L₁)ₐ-(Ar₁)_{b};

L₁ represents a single bond, a substituted or unsubstituted (C6-C30)arylene, or a substituted or unsubstituted (3- to 30-membered)heteroarylene:

Ar₁ represents a substituted or unsubstituted (C6-C30)aryl, a substituted or unsubstituted (3- to 30-membered)heteroaryl, or —N—(Ar₂)(Ar₃);

Ar₂ and Ar₃ each independently represent a substituted or unsubstituted (C1-C30)alkyl, a substituted or unsubstituted (C2-C30)alkenyl, a substituted or unsubstituted fused ring of an (C3-C30) aliphatic ring and a (C6-C30) aromatic ring, a substituted or unsubstituted (C6-C30)aryl, or a substituted or unsubstituted (3- to 30-membered)heteroaryl; and

a represents an integer of 1 or 2, and b represents an integer of 1 to 4; and when a and b are 2 or more, each of L₁ and each of Ar₁ may be the same or different;

provided that the compounds of formula 1 where R₅ to R₁₀ and R₁₂ represent hydrogen, and R₁₁ includes a substituted amino group, are excluded.

In one embodiment, R₁ to R₄ each independently may be *-(L₁)ₐ-(Ar₁)_{b}, a substituted or unsubstituted (C1-C30)alkyl, a substituted or unsubstituted (C6-C30)aryl, or a substituted or unsubstituted (3- to 30-membered)heteroaryl, preferably, *-(L₁)ₐ-(Ar₁)_{b}, a substituted or unsubstituted (C1-C10)alkyl, a substituted or unsubstituted (C6-C25)aryl, or a substituted or unsubstituted (5- to 25-membered)heteroaryl, more preferably *-(L₁)ₐ-(Ar₁)_{b}, a substituted or unsubstituted (C1-C4)alkyl, a substituted or unsubstituted (C6-C18)aryl, or a substituted or unsubstituted (5- to 18-membered)heteroaryl. For example, R₁ to R₄ each independently may be a substituted or unsubstituted methyl, a substituted or unsubstituted phenyl, a substituted or unsubstituted biphenyl, a substituted or unsubstituted pyridyl, a substituted or unsubstituted dibenzothiophenyl, a substituted or unsubstituted dibenzofuranyl, or *-(L₁)ₐ-(Ar₁)_{b}.

In one embodiment, R₅ to R₁₂ each independently may be *-(L₁)ₐ-(Ar₁)_{b}, hydrogen, a substituted or unsubstituted fused ring of an (C3-C30) aliphatic ring and a (C6-C30) aromatic ring, a substituted or unsubstituted (C6-C30)aryl, or a substituted or unsubstituted (3- to 30-membered)heteroaryl; or may be linked to an adjacent substituent(s) to form a ring(s), preferably *-(L₁)ₐ-(Ar₁)_{b}, hydrogen, a substituted or unsubstituted (C6-C25)aryl, or a substituted or unsubstituted (5- to 25-membered)heteroaryl; or may be linked to an adjacent substituent(s) to form a substituted or unsubstituted (5- to 30-membered) monocylic or polycyclic, aliphatic or aromatic ring(s) or a combination thereof, more preferably *-(L₁)ₐ-(Ar₁)_{b}, hydrogen, a substituted or unsubstituted (C6-C18)aryl, or a substituted or unsubstituted (5- to 18-membered)heteroaryl; or may be linked to an adjacent substituent(s) to form a substituted or unsubstituted (5- to 30-membered) monocylic or polycyclic, aromatic ring(s).

In formula 1 above, at least one of R₁ to R₁₂ represent(s) *-(L₁)ₐ-(Ar₁)_{b}, for example, at least one of R₁ to R₄, at least one of R₅ to R₈, or at least one of R₉ to R₁₂ may be *-(L₁)ₐ-(Ar₁)_{b}. For example, R₁ to R₄ other than *-(L₁)ₐ-(Ar₁)_{b} among R₁ to R₄ each independently may be a substituted or unsubstituted methyl, a substituted or unsubstituted phenyl, a substituted or unsubstituted biphenyl, a substituted or unsubstituted dibenzofuranyl, or a substituted or unsubstituted dibenzothiophenyl. For example, R₅ to R₁₂ other than *-(L₁)ₐ-(Ar₁)_{b} among R₅ to R₁₂ each independently may be hydrogen, phenyl unsubstituted or substituted with (C6-C30)aryl or deuterium, a substituted or unsubstituted m-biphenyl, or a substituted or unsubstituted pyridyl; or the substituent(s) adjacent to R₅ to R₁₂, or the substituent(s) adjacent to R₉ to R₁₂ may be linked to each other to form a benzene ring, a naphthalene ring, or a phenanthene ring.

According to one embodiment, the formula 1 may be an organic electroluminescent compound where Ar₁ represents a substituted or unsubstituted (3- to 30-membered)heteroaryl containing at least one N, or —N—(Ar₂)(Ar₃); and L₁ represents a single bond or a substituted or unsubstituted (C6-C30)arylene.

According to one embodiment, the organic electroluminescent compound represented by formula 1 may be represented by any one of the following formulas 1-1 to 1-4.

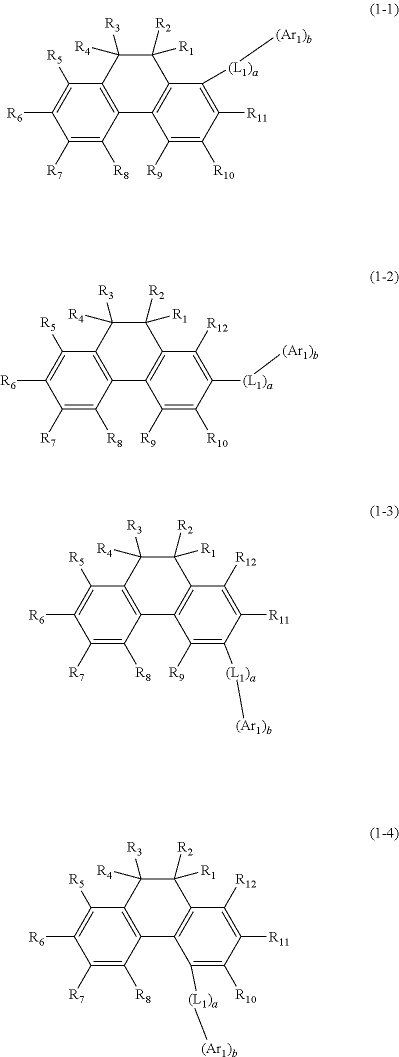

In formulas 1-1 to 1-4,

R₁ to R₁₂, L₁, Ar₁, a, and b are as defined in formula 1 above.

According to another embodiment, the organic electroluminescent compound represented by formula 1 may be represented by any one of the following formulas 1-5 to 1-13.

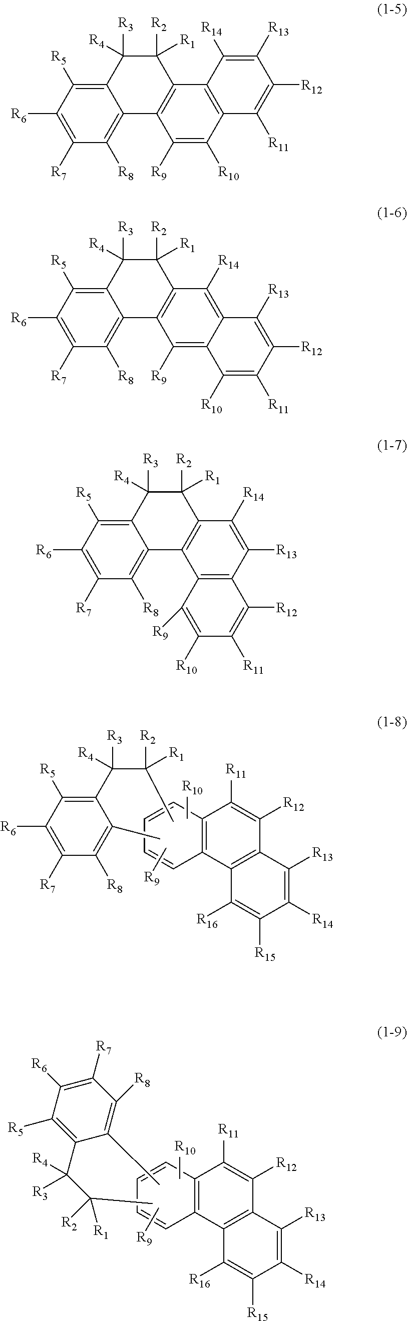

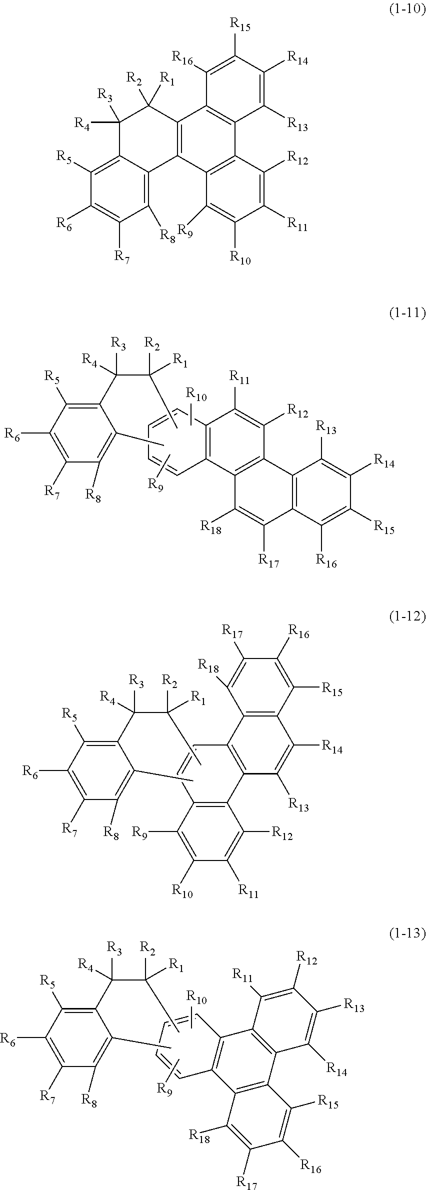

In formulas 1-5 to 1-13,

R₁ to R₁₂ are as defined in formula 1 above;

R₁₃ to R₁₈ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of an (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s):

provided that at least one of R₁ to R₁₄ in the formulas 1-5 to 1-7, at least one of R₁ to R₁₈ in the formulas 1-8 to 1-10, and at least one of R₁ to R₁₈ in the formulas 1-11 to 1-13 represent(s) *-(L₁)ₐ-(Ar₁)^{b}; and

L₁, Ar₁, a, and b are as defined in formula 1 above.

In one embodiment, Ar₁ may be a substituted or unsubstituted (C6-C30)aryl, a substituted or unsubstituted (5- to 30-membered)heteroaryl, or —N—(Ar₂)(Ar₃), preferably, a substituted or unsubstituted (5- to 25-membered)heteroaryl containing at least one N, or —N—(Ar₂)(Ar₃), more preferably, a substituted or unsubstituted (5- to 25-membered)heteroaryl containing at least one N, or —N—(Ar₂)(Ar₃). Wherein, Ar₂ and Ar₃ each independently may be a substituted or unsubstituted fused ring of an (C3-C30) aliphatic ring and a (C6-C30) aromatic ring, a substituted or unsubstituted (C6-C30)aryl, or a substituted or unsubstituted (3- to 30-membered)heteroaryl, preferably, a substituted or unsubstituted fused ring of an (C3-C20) aliphatic ring and a (C6-C25) aromatic ring, a substituted or unsubstituted (C6-C25)aryl, or a substituted or unsubstituted (5- to 25-membered)heteroaryl, more preferably, a substituted or unsubstituted fused ring of a (C3-C10) aliphatic ring and a (C6-C18) aromatic ring, a substituted or unsubstituted (C6-C18)aryl, or a substituted or unsubstituted (5- to 18-membered)heteroaryl. For example, Ar₂ and Ar₃ each independently may be a substituted or unsubstituted phenyl, a substituted or unsubstituted naphthyl, a substituted or unsubstituted p-biphenyl, a substituted or unsubstituted m-biphenyl, a substituted or unsubstituted fluorenyl, a substituted or unsubstituted phenanthrenyl, a substituted or unsubstituted chrysenyl, a substituted or unsubstituted dibenzofuranyl, a substituted or unsubstituted dibenzothiophenyl, a substituted or unsubstituted carbazolyl, a substituted or unsubstituted benzofluorenyl, or a substituted or unsubstituted dihydrophenanthrenyl.

In one embodiment, a substituted or unsubstituted (C6-C30)aryl in Ar₁ may be a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted triphenylenyl, or a substituted or unsubstituted phenanthrenyl, preferably, phenyl unsubstituted or substituted with deuterium or (5- to 30-membered)heteroaryl, a substituted or unsubstituted p-biphenyl, a substituted or unsubstituted m-biphenyl, a substituted or unsubstituted m-terphenyl, or a substituted or unsubstituted naphthyl.

In one embodiment, the substituted or unsubstituted (3- to 30-membered)heteroaryl in Ar₁ may be a substituted or unsubstituted pyridyl, a substituted or unsubstituted pyrimidinyl, a substituted or unsubstituted triazinyl, a substituted or unsubstituted pyrazinyl, a substituted or unsubstituted quinolinyl, a substituted or unsubstituted quinazolinyl, a substituted or unsubstituted quinoxalinyl, a substituted or unsubstituted benzoquinolinyl, a substituted or unsubstituted benzoquinazolinyl, a substituted or unsubstituted benzoquinoxalinyl, a substituted or unsubstituted dibenzoquinolinyl, a substituted or unsubstituted dibenzoquinazolinyl, a substituted or unsubstituted dibenzoquinoxalinyl, a substituted or unsubstituted indenopyridyl, a substituted or unsubstituted indenopyrimidinyl, a substituted or unsubstituted indenopyrazinyl, a substituted or unsubstituted benzofuropyridyl, a substituted or unsubstituted benzofuropyrimidinyl, a substituted or unsubstituted benzofuropyrazinyl, a substituted or unsubstituted benzothiopyridyl, a substituted or unsubstituted benzothiopyrimidinyl, a substituted or unsubstituted benzothiopyrazinyl, a substituted or unsubstituted carbazolyl, a substituted or unsubstituted benzocarbazolyl, a substituted or unsubstituted dibenzofuranyl, or a substituted or unsubstituted dibenzothiophenyl, preferably, a substituted or unsubstituted pyridyl, carbazolyl unsubstituted or substituted with (C6-C30)aryl, a substituted or unsubstituted benzocarbazolyl, quinazolinyl unsubstituted or substituted with (C6-C30)aryl and/or (5- to 30-membered)heteroaryl, quinoxalinyl unsubstituted or substituted with (C6-C30)aryl and/or (5- to 30-membered)heteroaryl, benzoquinoxalinyl unsubstituted or substituted with (C6-C30)aryl, a substituted or unsubstituted dibenzofuranyl, a substituted or unsubstituted dibenzothiophenyl, or triazinyl unsubstituted or substituted with (C6-C30)aryl and/or (5- to 30-membered)heteroaryl.

In one embodiment, L₁ may be a single bond, a substituted or unsubstituted (C6-C30)arylene, or a substituted or unsubstituted (5- to 30-membered)heteroarylene, preferably, a single bond, a substituted or unsubstituted (C6-C25)arylene, or a substituted or unsubstituted (5- to 25-membered)heteroarylene, more preferably, a single bond, or a substituted or unsubstituted (C6-C18)arylene, or a substituted or unsubstituted (5- to 18-membered)heteroarylene. For example, L₁ may be a single bond, or a substituted or unsubstituted phenylene, a substituted or unsubstituted biphenylene, a substituted or unsubstituted terphenylene, a substituted or unsubstituted naphthylene, a substituted or unsubstituted phenanthrenylene, a substituted or unsubstituted triphenylenylene, a substituted or unsubstituted fluorenylene, a substituted or unsubstituted pyridylene, a substituted or unsubstituted triazinylene, a substituted or unsubstituted carbazolylene, a substituted or unsubstituted quinoxalinylene, a substituted or unsubstituted quinazolinylene, a substituted or unsubstituted dibenzofuranylene, or a substituted or unsubstituted benzoquinoxalinylene, preferably, a substituted or unsubstituted phenylene, a substituted or unsubstituted p-biphenylene, a substituted or unsubstituted m-biphenylene, a substituted or unsubstituted o-biphenylene, a substituted or unsubstituted naphthylene, a substituted or unsubstituted pyridylene, a substituted or unsubstituted triazinylene, a substituted or unsubstituted carbazolylene, a substituted or unsubstituted quinoxalinylene, a substituted or unsubstituted quinazolinylene, a substituted or unsubstituted dibenzofuranylene, or a substituted or unsubstituted benzoquinoxalinylene.

In one embodiment, a may be an integer of 1 or 2, b may be an integer of 1 or 2, and when a and b are 2, each of L₁ and each of Ar₁ may be the same or different.

According to one embodiment, the organic electroluminescent compound represented by formula 1 above may be more specifically illustrated by the following compounds, but is not limited thereto.

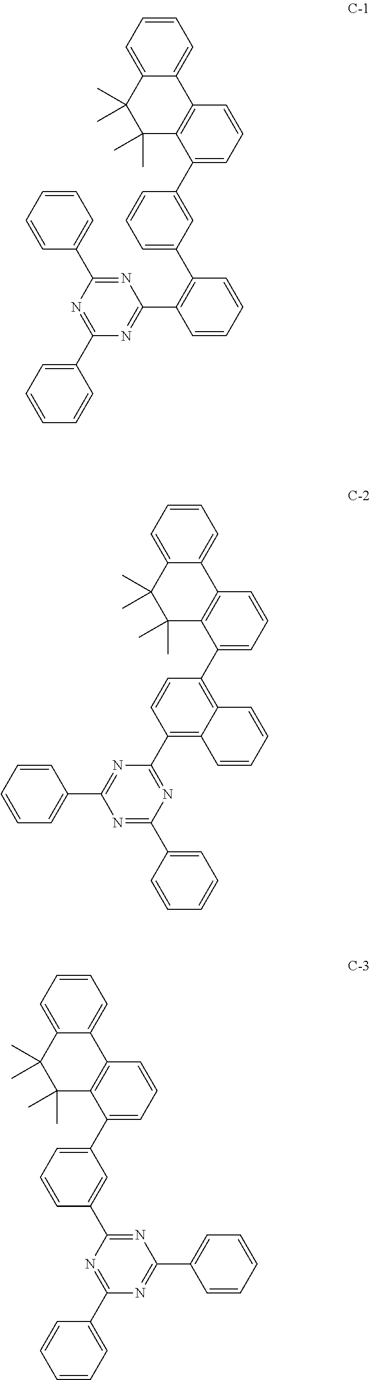 [SMILES: CC1(C)c2ccccc2-c2cccc(-c3cccc(-c4ccccc4-c4nc(-c5ccccc5)nc(-c5ccccc5)n4)c3)c2C1(C)C ; CC1(C)c2ccccc2-c2cccc(-c3ccc(-c4nc(-c5ccccc5)nc(-c5ccccc5)n4)c4ccccc34)c2C1(C)C ; CC1(C)c2ccccc2-c2cccc(-c3cccc(-c4nc(-c5ccccc5)nc(-c5ccccc5)n4)c3)c2C1(C)C]

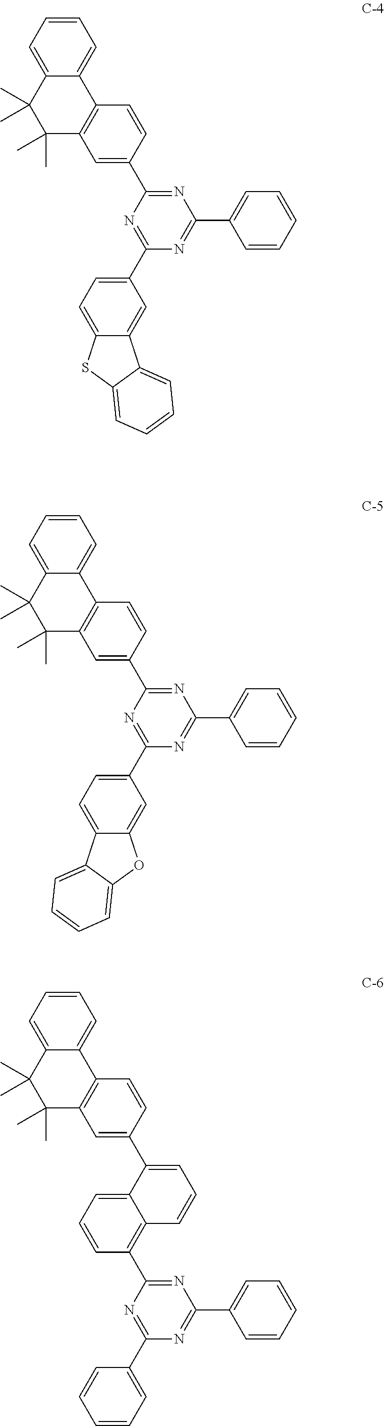 [SMILES: CC1(C)c2ccccc2-c2ccc(-c3nc(-c4ccccc4)nc(-c4ccc5sc6ccccc6c5c4)n3)cc2C1(C)C ; CC1(C)c2ccccc2-c2ccc(-c3nc(-c4ccccc4)nc(-c4ccc5c(c4)oc4ccccc45)n3)cc2C1(C)C ; CC1(C)c2ccccc2-c2ccc(-c3cccc4c(-c5nc(-c6ccccc6)nc(-c6ccccc6)n5)cccc34)cc2C1(C)C]

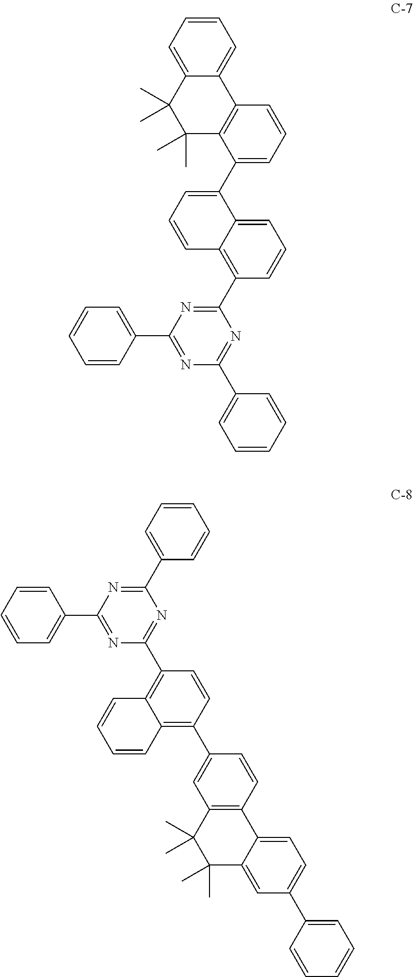 [SMILES: CC1(C)c2ccccc2-c2cccc(-c3cccc4c(-c5nc(-c6ccccc6)nc(-c6ccccc6)n5)cccc34)c2C1(C)C ; CC1(C)c2cc(-c3ccccc3)ccc2-c2ccc(-c3ccc(-c4nc(-c5ccccc5)nc(-c5ccccc5)n4)c4ccccc34)cc2C1(C)C]

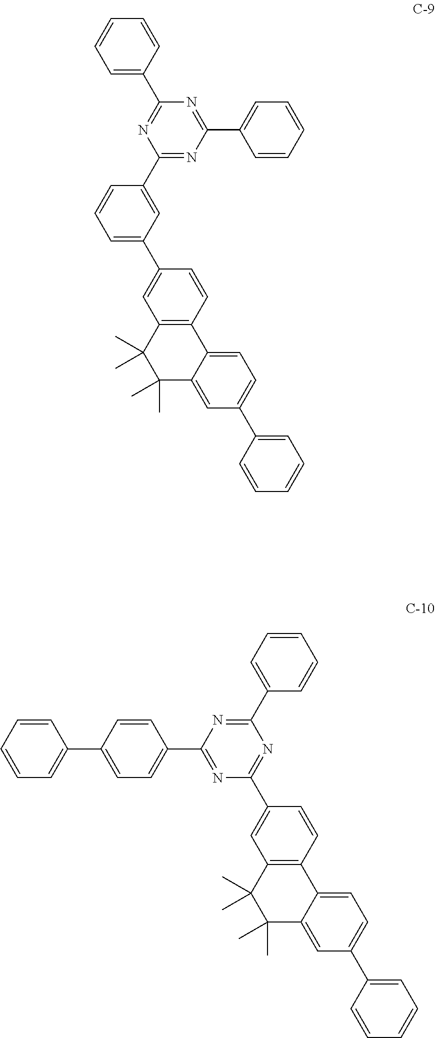 [SMILES: CC1(C)c2cc(-c3ccccc3)ccc2-c2ccc(-c3cccc(-c4nc(-c5ccccc5)nc(-c5ccccc5)n4)c3)cc2C1(C)C ; CC1(C)c2cc(-c3ccccc3)ccc2-c2ccc(-c3nc(-c4ccccc4)nc(-c4ccc(-c5ccccc5)cc4)n3)cc2C1(C)C]

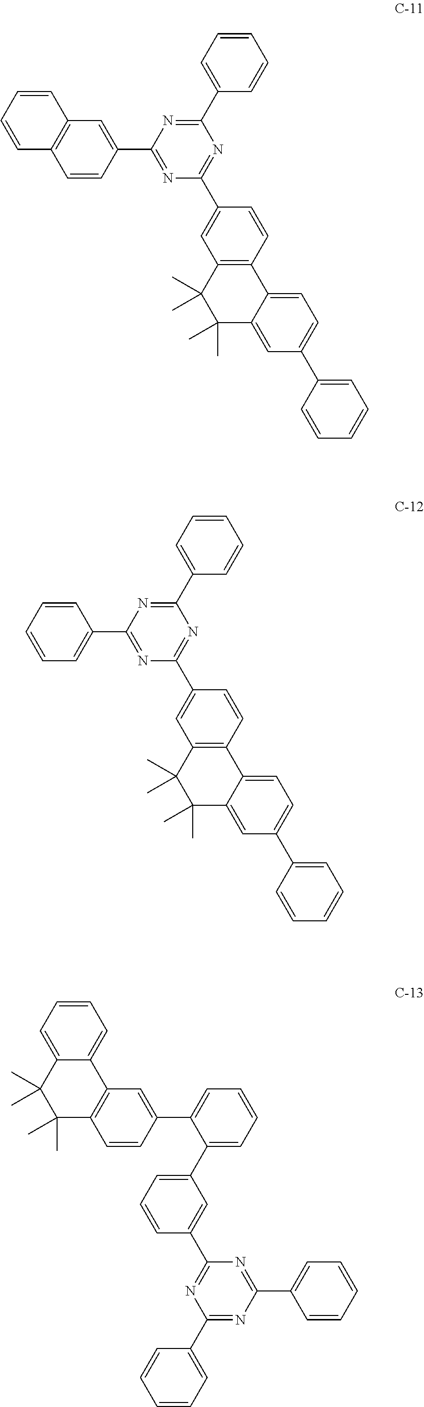 [SMILES: CC1(C)c2cc(-c3ccccc3)ccc2-c2ccc(-c3nc(-c4ccccc4)nc(-c4ccc5ccccc5c4)n3)cc2C1(C)C ; CC1(C)c2cc(-c3ccccc3)ccc2-c2ccc(-c3nc(-c4ccccc4)nc(-c4ccccc4)n3)cc2C1(C)C ; CC1(C)c2ccccc2-c2cc(-c3ccccc3-c3cccc(-c4nc(-c5ccccc5)nc(-c5ccccc5)n4)c3)ccc2C1(C)C]

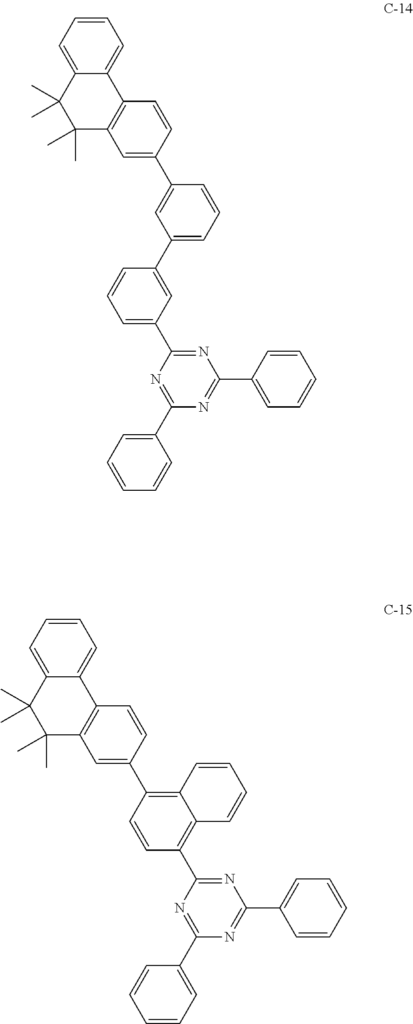 [SMILES: CC1(C)c2ccccc2-c2ccc(-c3cccc(-c4cccc(-c5nc(-c6ccccc6)nc(-c6ccccc6)n5)c4)c3)cc2C1(C)C ; CC1(C)c2ccccc2-c2ccc(-c3ccc(-c4nc(-c5ccccc5)nc(-c5ccccc5)n4)c4ccccc34)cc2C1(C)C]

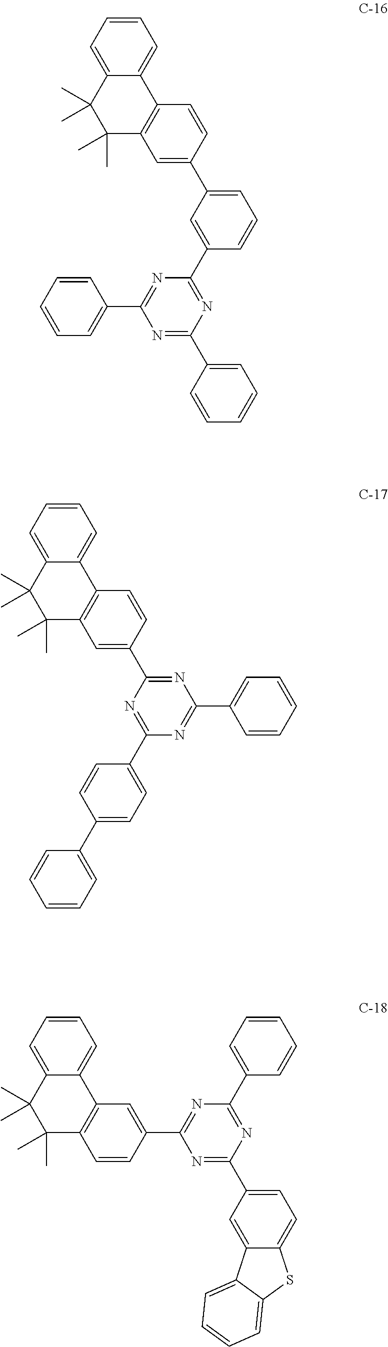 [SMILES: CC1(C)c2ccccc2-c2ccc(-c3cccc(-c4nc(-c5ccccc5)nc(-c5ccccc5)n4)c3)cc2C1(C)C ; CC1(C)c2ccccc2-c2ccc(-c3nc(-c4ccccc4)nc(-c4ccc(-c5ccccc5)cc4)n3)cc2C1(C)C ; CC1(C)c2ccccc2-c2cc(-c3nc(-c4ccccc4)nc(-c4ccc5sc6ccccc6c5c4)n3)ccc2C1(C)C]

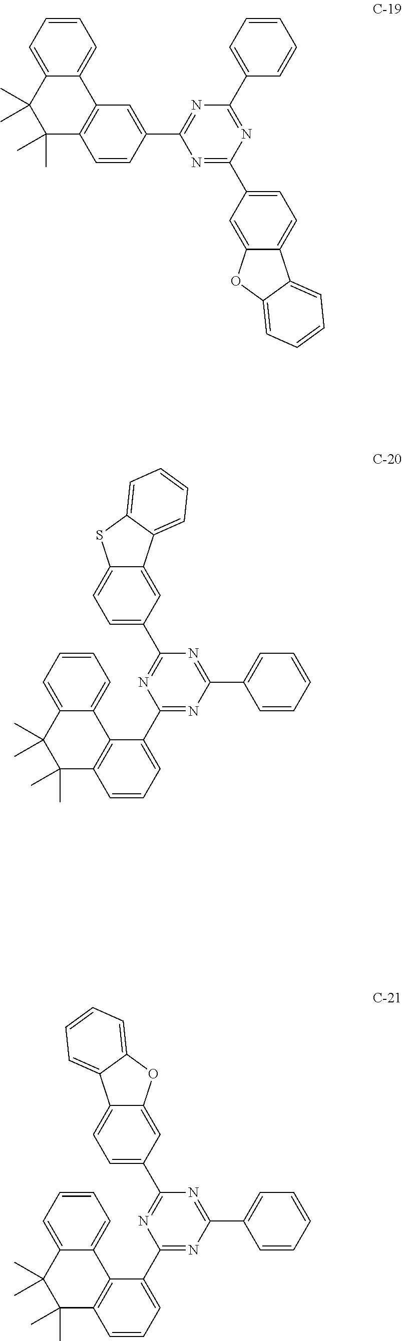 [SMILES: CC1(C)c2ccccc2-c2cc(-c3nc(-c4ccccc4)nc(-c4ccc5c(c4)oc4ccccc45)n3)ccc2C1(C)C ; CC1(C)c2ccccc2-c2c(-c3nc(-c4ccccc4)nc(-c4ccc5sc6ccccc6c5c4)n3)cccc2C1(C)C ; CC1(C)c2ccccc2-c2c(-c3nc(-c4ccccc4)nc(-c4ccc5c(c4)oc4ccccc45)n3)cccc2C1(C)C]

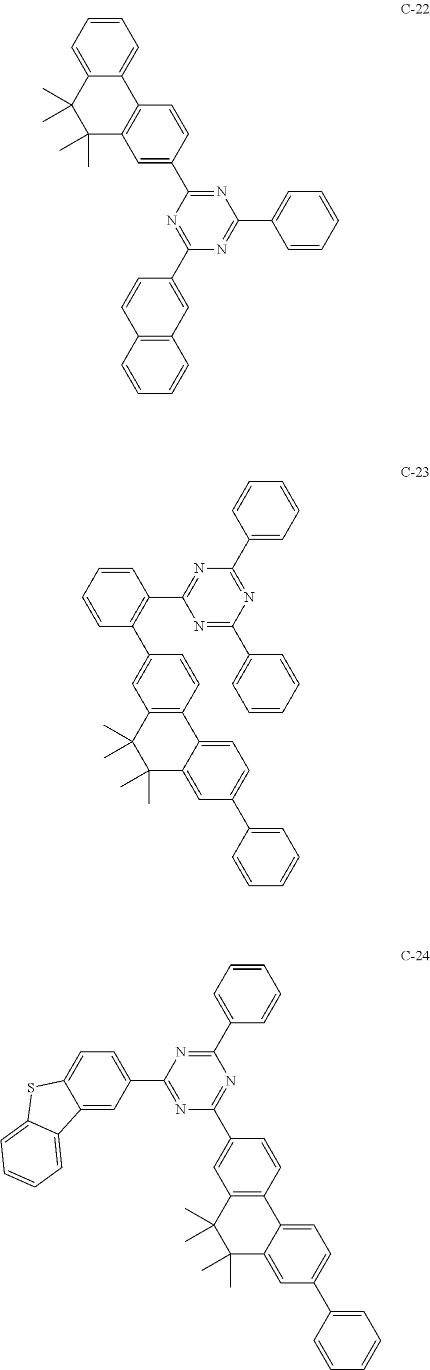 [SMILES: CC1(C)c2ccccc2-c2ccc(-c3nc(-c4ccccc4)nc(-c4ccc5ccccc5c4)n3)cc2C1(C)C ; CC1(C)c2cc(-c3ccccc3)ccc2-c2ccc(-c3ccccc3-c3nc(-c4ccccc4)nc(-c4ccccc4)n3)cc2C1(C)C ; CC1(C)c2cc(-c3ccccc3)ccc2-c2ccc(-c3nc(-c4ccccc4)nc(-c4ccc5sc6ccccc6c5c4)n3)cc2C1(C)C]

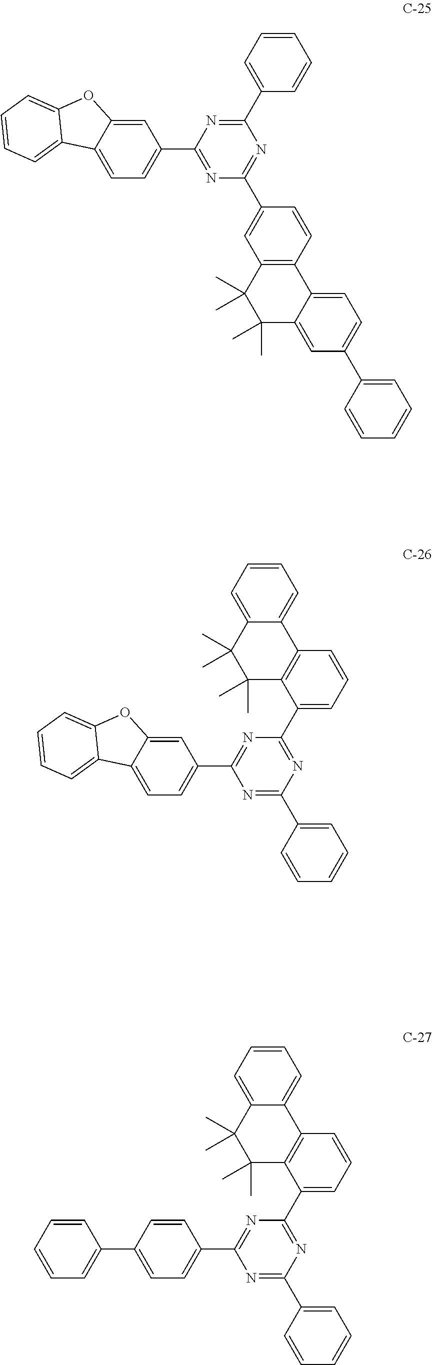 [SMILES: CC1(C)c2cc(-c3ccccc3)ccc2-c2ccc(-c3nc(-c4ccccc4)nc(-c4ccc5c(c4)oc4ccccc45)n3)cc2C1(C)C ; CC1(C)c2ccccc2-c2cccc(-c3nc(-c4ccccc4)nc(-c4ccc5c(c4)oc4ccccc45)n3)c2C1(C)C ; CC1(C)c2ccccc2-c2cccc(-c3nc(-c4ccccc4)nc(-c4ccc(-c5ccccc5)cc4)n3)c2C1(C)C]

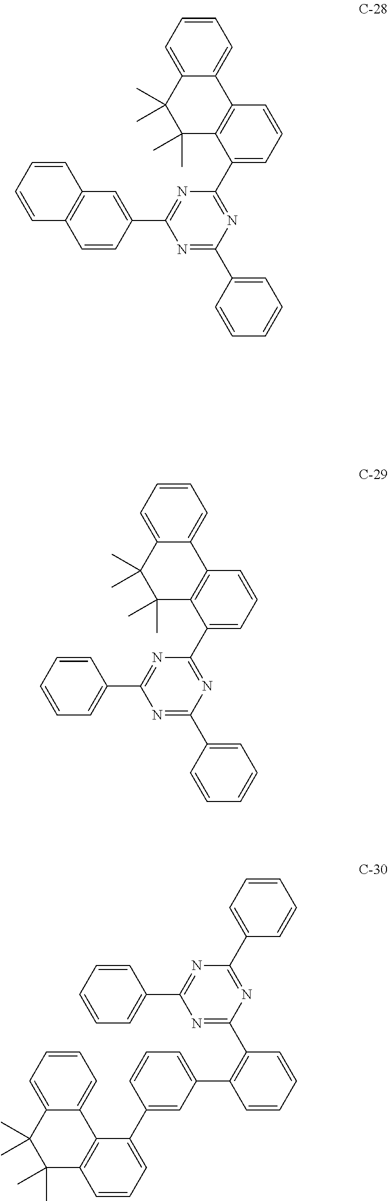 [SMILES: CC1(C)c2ccccc2-c2cccc(-c3nc(-c4ccccc4)nc(-c4ccc5ccccc5c4)n3)c2C1(C)C ; CC1(C)c2ccccc2-c2cccc(-c3nc(-c4ccccc4)nc(-c4ccccc4)n3)c2C1(C)C ; CC1(C)c2ccccc2-c2c(-c3cccc(-c4ccccc4-c4nc(-c5ccccc5)nc(-c5ccccc5)n4)c3)cccc2C1(C)C]

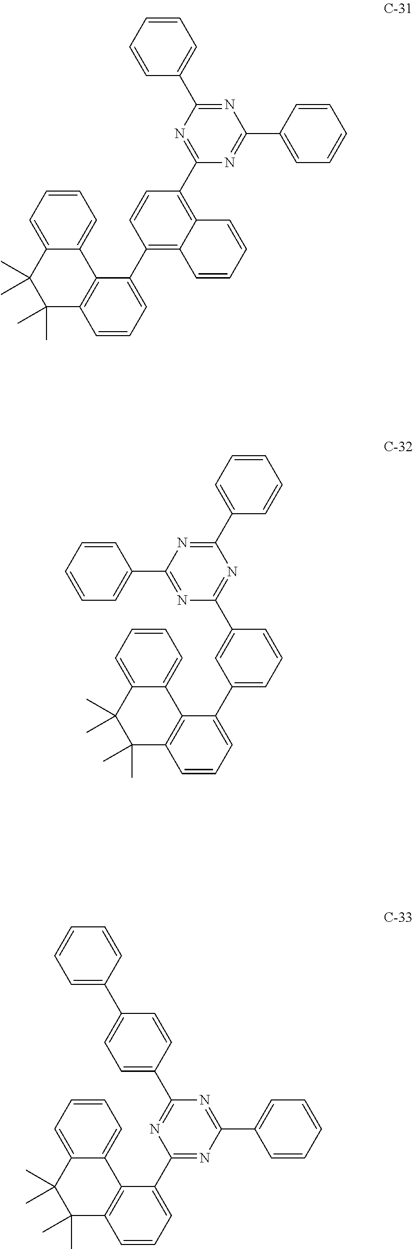 [SMILES: CC1(C)c2ccccc2-c2c(-c3ccc(-c4nc(-c5ccccc5)nc(-c5ccccc5)n4)c4ccccc34)cccc2C1(C)C ; CC1(C)c2ccccc2-c2c(-c3cccc(-c4nc(-c5ccccc5)nc(-c5ccccc5)n4)c3)cccc2C1(C)C ; CC1(C)c2ccccc2-c2c(-c3nc(-c4ccccc4)nc(-c4ccc(-c5ccccc5)cc4)n3)cccc2C1(C)C]

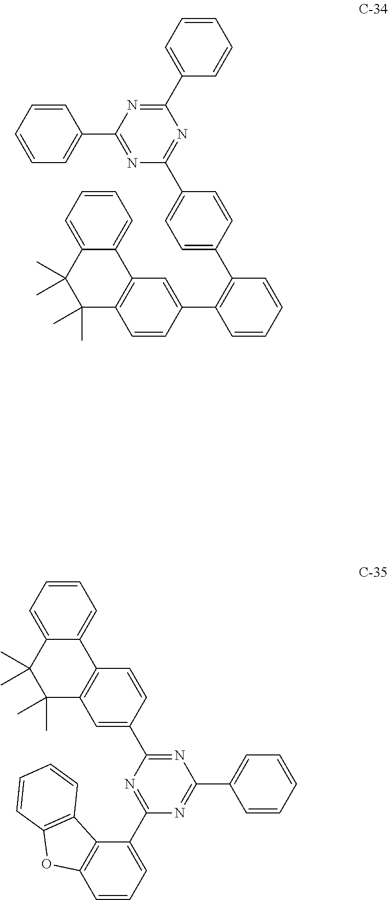 [SMILES: CC1(C)c2ccccc2-c2cc(-c3ccccc3-c3ccc(-c4nc(-c5ccccc5)nc(-c5ccccc5)n4)cc3)ccc2C1(C)C ; CC1(C)c2ccccc2-c2ccc(-c3nc(-c4ccccc4)nc(-c4cccc5oc6ccccc6c45)n3)cc2C1(C)C]

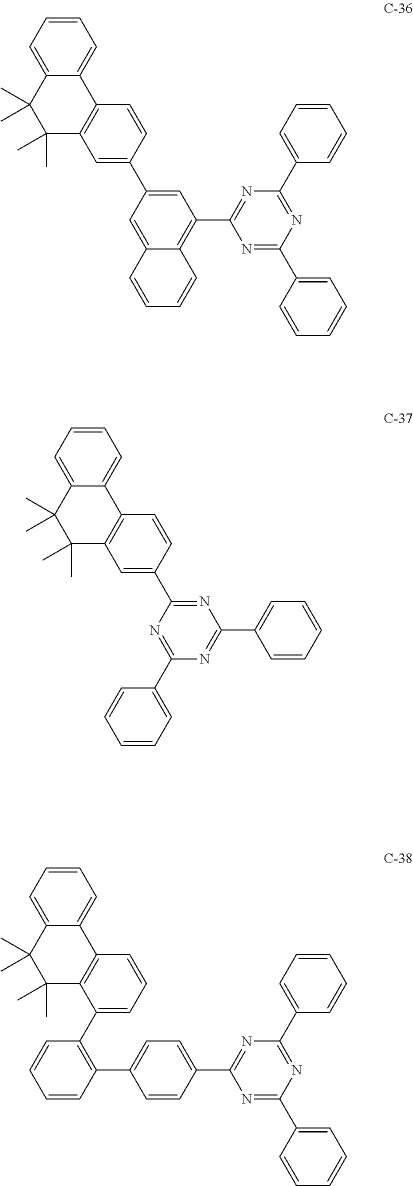 [SMILES: CC1(C)c2ccccc2-c2ccc(-c3cc(-c4nc(-c5ccccc5)nc(-c5ccccc5)n4)c4ccccc4c3)cc2C1(C)C ; CC1(C)c2ccccc2-c2ccc(-c3nc(-c4ccccc4)nc(-c4ccccc4)n3)cc2C1(C)C ; CC1(C)c2ccccc2-c2cccc(-c3ccccc3-c3ccc(-c4nc(-c5ccccc5)nc(-c5ccccc5)n4)cc3)c2C1(C)C]

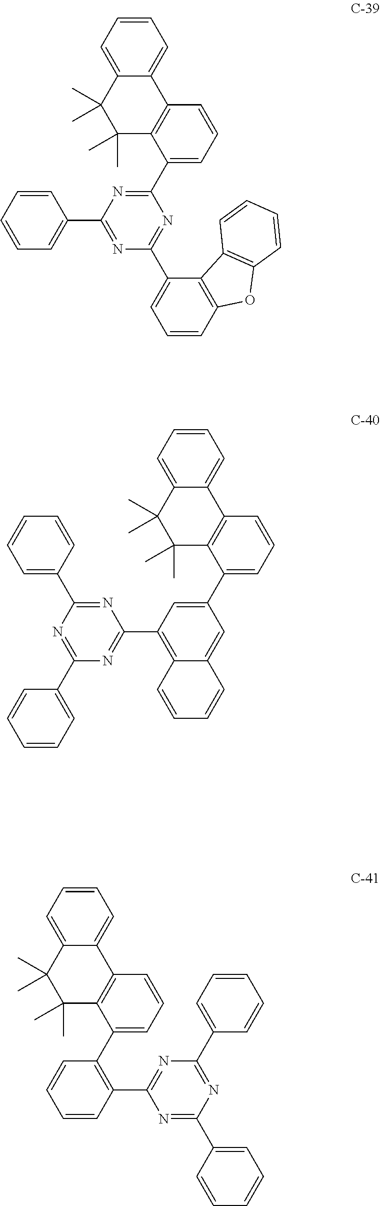 [SMILES: CC1(C)c2ccccc2-c2cccc(-c3nc(-c4ccccc4)nc(-c4cccc5oc6ccccc6c45)n3)c2C1(C)C ; CC1(C)c2ccccc2-c2cccc(-c3cc(-c4nc(-c5ccccc5)nc(-c5ccccc5)n4)c4ccccc4c3)c2C1(C)C ; CC1(C)c2ccccc2-c2cccc(-c3ccccc3-c3nc(-c4ccccc4)nc(-c4ccccc4)n3)c2C1(C)C]

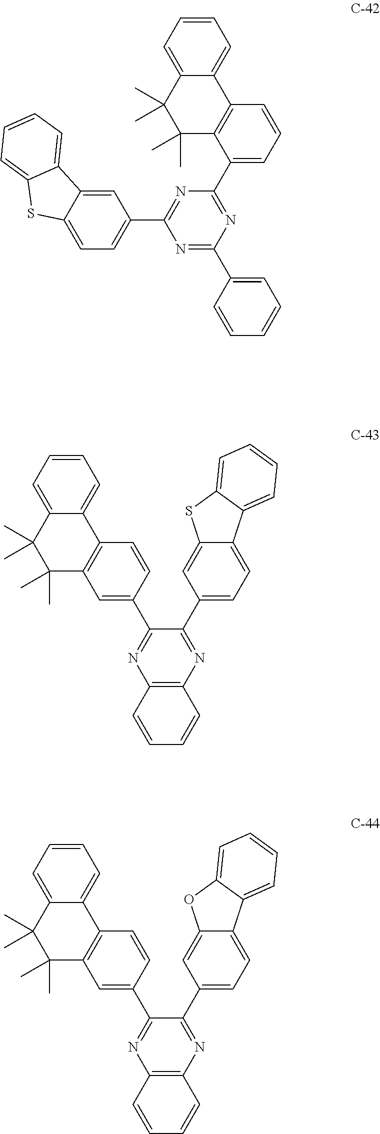 [SMILES: CC1(C)c2ccccc2-c2cccc(-c3nc(-c4ccccc4)nc(-c4ccc5sc6ccccc6c5c4)n3)c2C1(C)C ; CC1(C)c2ccccc2-c2ccc(-c3nc4ccccc4nc3-c3ccc4c(c3)sc3ccccc34)cc2C1(C)C ; CC1(C)c2ccccc2-c2ccc(-c3nc4ccccc4nc3-c3ccc4c(c3)oc3ccccc34)cc2C1(C)C]

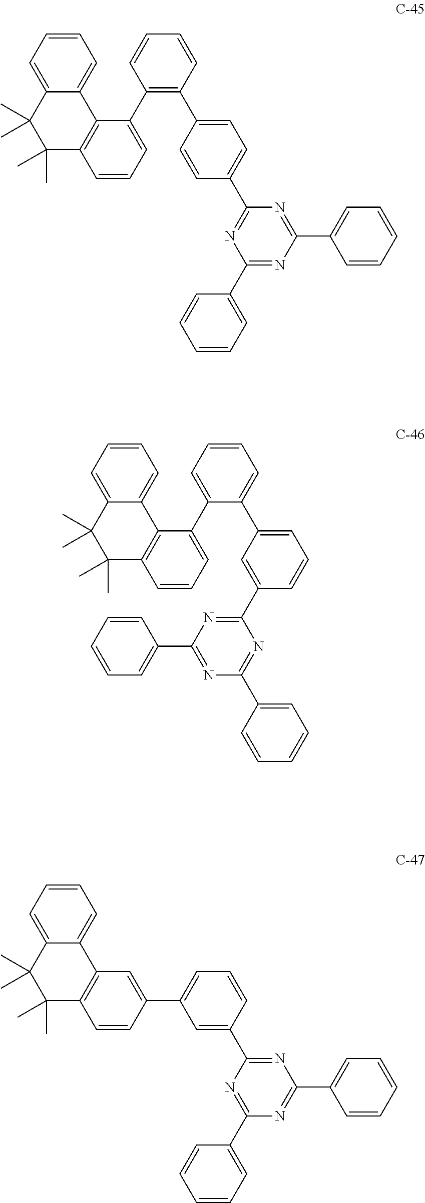 [SMILES: CC1(C)c2ccccc2-c2c(-c3ccccc3-c3ccc(-c4nc(-c5ccccc5)nc(-c5ccccc5)n4)cc3)cccc2C1(C)C ; CC1(C)c2ccccc2-c2c(-c3ccccc3-c3cccc(-c4nc(-c5ccccc5)nc(-c5ccccc5)n4)c3)cccc2C1(C)C ; CC1(C)c2ccccc2-c2cc(-c3cccc(-c4nc(-c5ccccc5)nc(-c5ccccc5)n4)c3)ccc2C1(C)C]

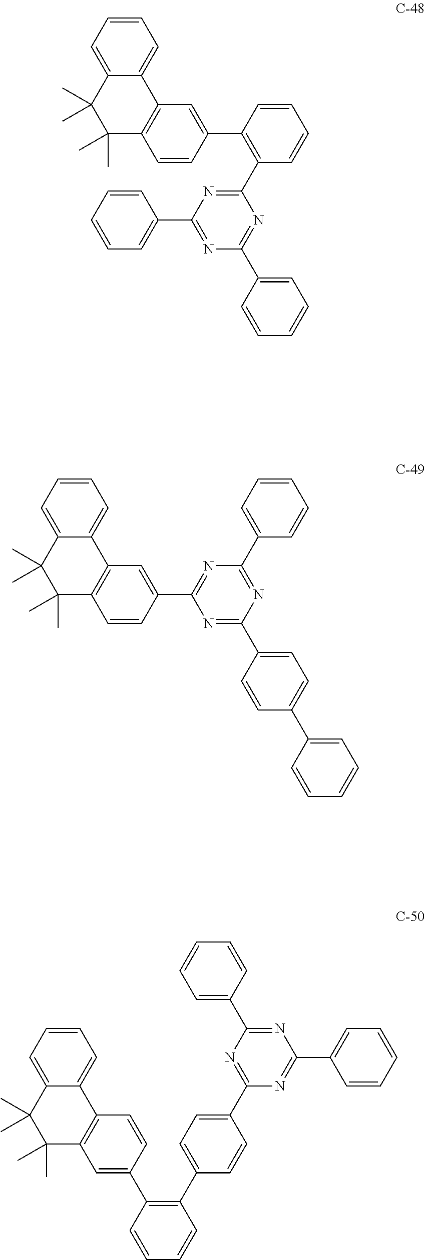 [SMILES: CC1(C)c2ccccc2-c2cc(-c3ccccc3-c3nc(-c4ccccc4)nc(-c4ccccc4)n3)ccc2C1(C)C ; CC1(C)c2ccccc2-c2cc(-c3nc(-c4ccccc4)nc(-c4ccc(-c5ccccc5)cc4)n3)ccc2C1(C)C ; CC1(C)c2ccccc2-c2ccc(-c3ccccc3-c3ccc(-c4nc(-c5ccccc5)nc(-c5ccccc5)n4)cc3)cc2C1(C)C]

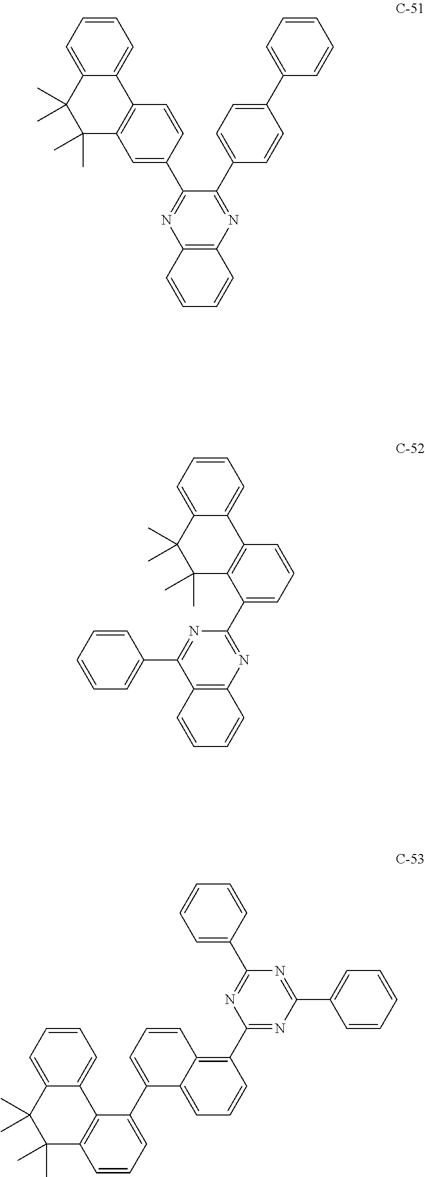 [SMILES: CC1(C)c2ccccc2-c2ccc(-c3nc4ccccc4nc3-c3ccc(-c4ccccc4)cc3)cc2C1(C)C ; CC1(C)c2ccccc2-c2cccc(-c3nc(-c4ccccc4)c4ccccc4n3)c2C1(C)C ; CC1(C)c2ccccc2-c2c(-c3cccc4c(-c5nc(-c6ccccc6)nc(-c6ccccc6)n5)cccc34)cccc2C1(C)C]

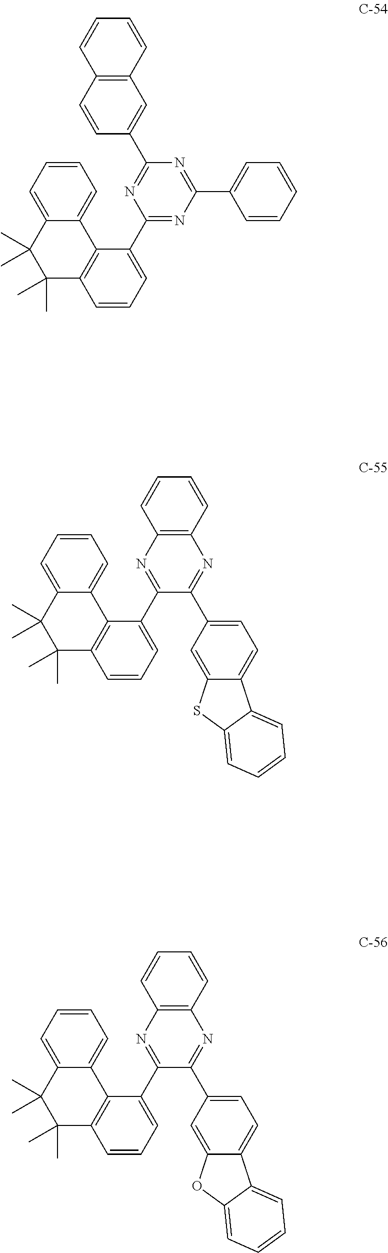 [SMILES: CC1(C)c2ccccc2-c2c(-c3nc(-c4ccccc4)nc(-c4ccc5ccccc5c4)n3)cccc2C1(C)C ; CC1(C)c2ccccc2-c2c(-c3nc4ccccc4nc3-c3ccc4c(c3)sc3ccccc34)cccc2C1(C)C ; CC1(C)c2ccccc2-c2c(-c3nc4ccccc4nc3-c3ccc4c(c3)oc3ccccc34)cccc2C1(C)C]

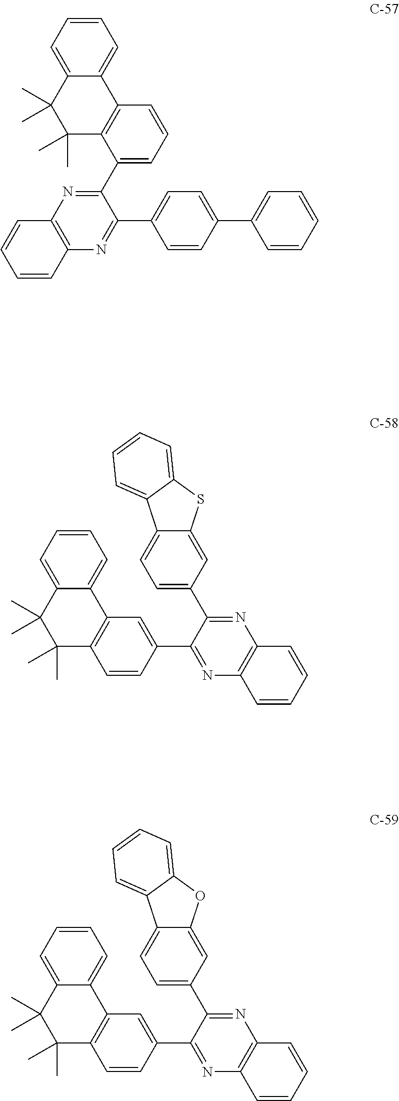 [SMILES: CC1(C)c2ccccc2-c2cccc(-c3nc4ccccc4nc3-c3ccc(-c4ccccc4)cc3)c2C1(C)C ; CC1(C)c2ccccc2-c2cc(-c3nc4ccccc4nc3-c3ccc4c(c3)sc3ccccc34)ccc2C1(C)C ; CC1(C)c2ccccc2-c2cc(-c3nc4ccccc4nc3-c3ccc4c(c3)oc3ccccc34)ccc2C1(C)C]

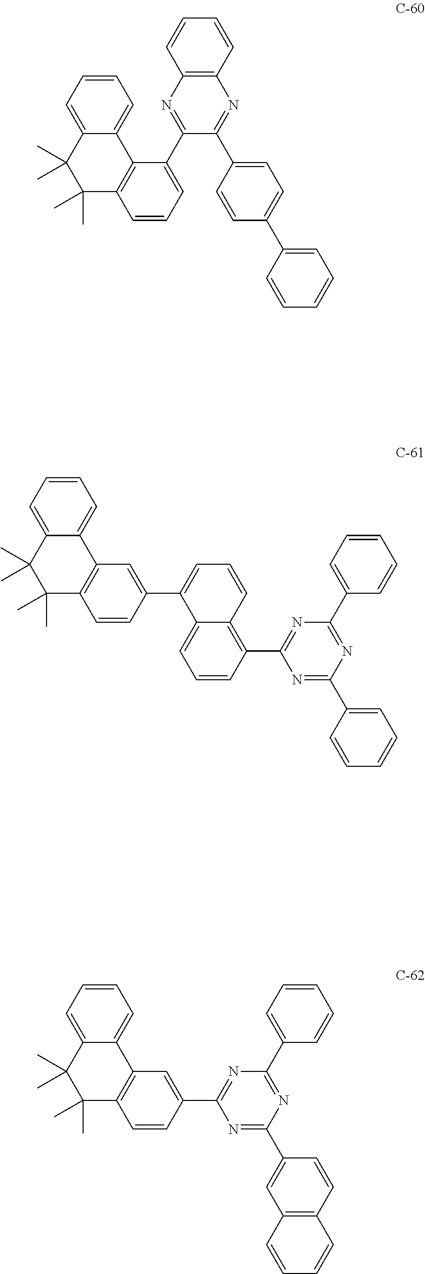 [SMILES: CC1(C)c2ccccc2-c2c(-c3nc4ccccc4nc3-c3ccc(-c4ccccc4)cc3)cccc2C1(C)C ; CC1(C)c2ccccc2-c2cc(-c3cccc4c(-c5nc(-c6ccccc6)nc(-c6ccccc6)n5)cccc34)ccc2C1(C)C ; CC1(C)c2ccccc2-c2cc(-c3nc(-c4ccccc4)nc(-c4ccc5ccccc5c4)n3)ccc2C1(C)C]

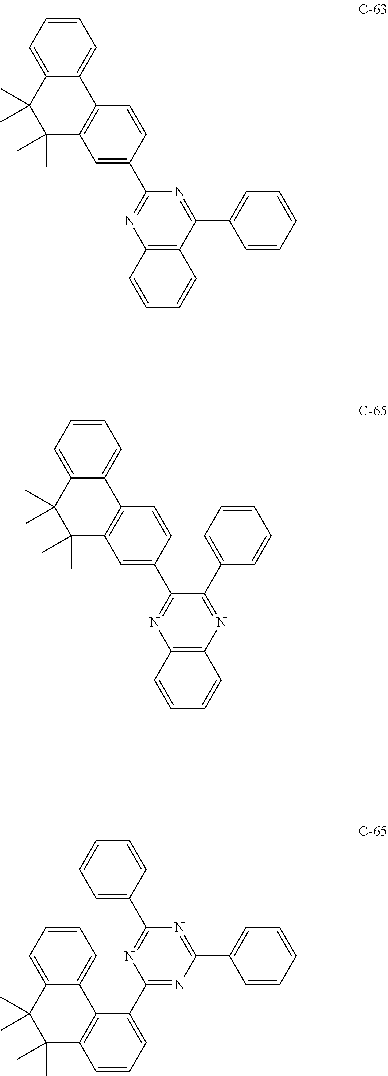 [SMILES: CC1(C)c2ccccc2-c2ccc(-c3nc(-c4ccccc4)c4ccccc4n3)cc2C1(C)C ; CC1(C)c2ccccc2-c2ccc(-c3nc4ccccc4nc3-c3ccccc3)cc2C1(C)C ; CC1(C)c2ccccc2-c2c(-c3nc(-c4ccccc4)nc(-c4ccccc4)n3)cccc2C1(C)C]

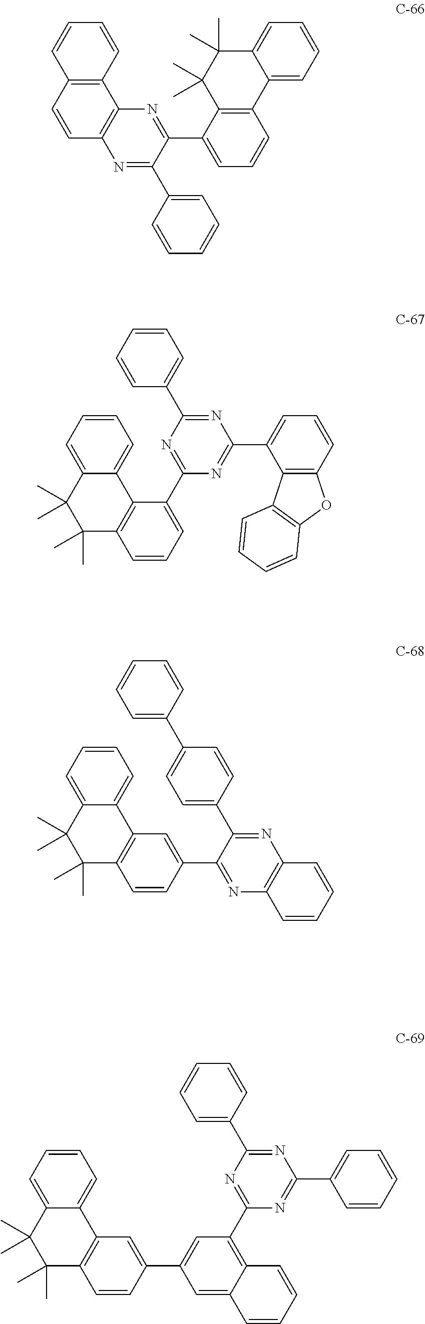 [SMILES: CC1(C)c2ccccc2-c2cccc(-c3nc4c(ccc5ccccc54)nc3-c3ccccc3)c2C1(C)C ; CC1(C)c2ccccc2-c2c(-c3nc(-c4ccccc4)nc(-c4cccc5oc6ccccc6c45)n3)cccc2C1(C)C ; CC1(C)c2ccccc2-c2cc(-c3nc4ccccc4nc3-c3ccc(-c4ccccc4)cc3)ccc2C1(C)C ; CC1(C)c2ccccc2-c2cc(-c3cc(-c4nc(-c5ccccc5)nc(-c5ccccc5)n4)c4ccccc4c3)ccc2C1(C)C]

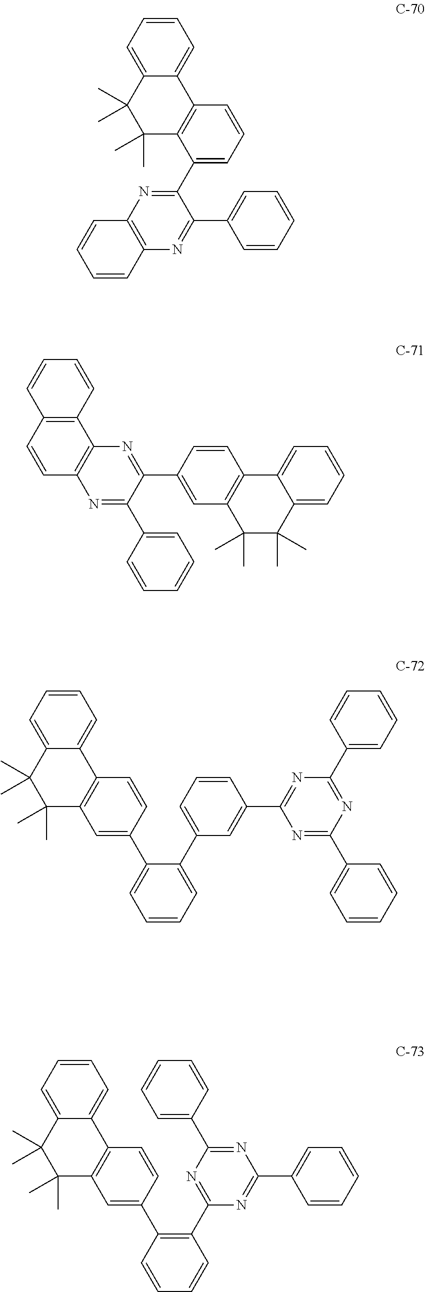 [SMILES: CC1(C)c2ccccc2-c2cccc(-c3nc4ccccc4nc3-c3ccccc3)c2C1(C)C ; CC1(C)c2ccccc2-c2ccc(-c3nc4c(ccc5ccccc54)nc3-c3ccccc3)cc2C1(C)C ; CC1(C)c2ccccc2-c2ccc(-c3ccccc3-c3cccc(-c4nc(-c5ccccc5)nc(-c5ccccc5)n4)c3)cc2C1(C)C ; CC1(C)c2ccccc2-c2ccc(-c3ccccc3-c3nc(-c4ccccc4)nc(-c4ccccc4)n3)cc2C1(C)C]

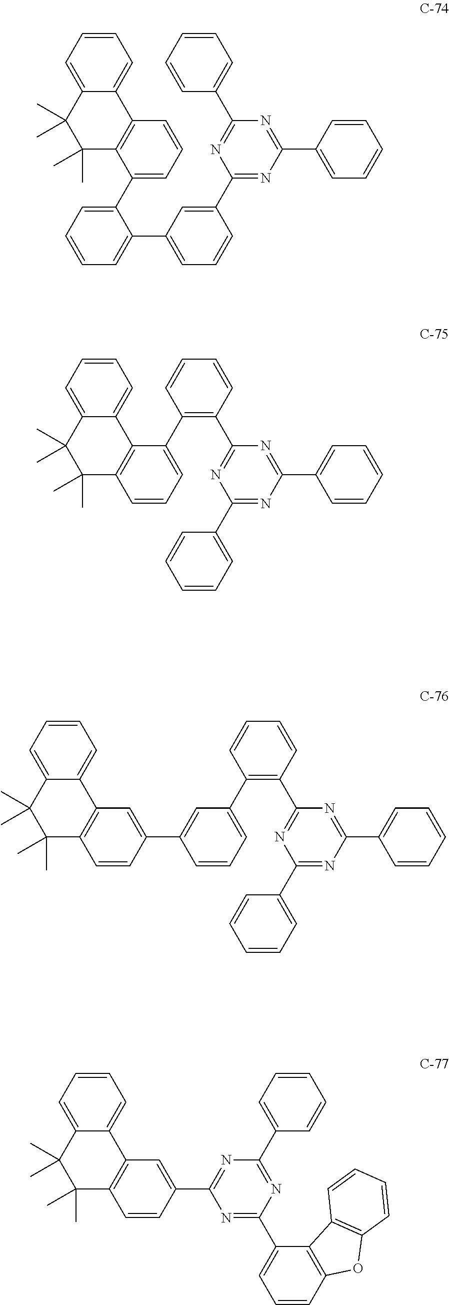 [SMILES: CC1(C)c2ccccc2-c2cccc(-c3ccccc3-c3cccc(-c4nc(-c5ccccc5)nc(-c5ccccc5)n4)c3)c2C1(C)C ; CC1(C)c2ccccc2-c2c(-c3ccccc3-c3nc(-c4ccccc4)nc(-c4ccccc4)n3)cccc2C1(C)C ; CC1(C)c2ccccc2-c2cc(-c3cccc(-c4ccccc4-c4nc(-c5ccccc5)nc(-c5ccccc5)n4)c3)ccc2C1(C)C ; CC1(C)c2ccccc2-c2cc(-c3nc(-c4ccccc4)nc(-c4cccc5oc6ccccc6c45)n3)ccc2C1(C)C]

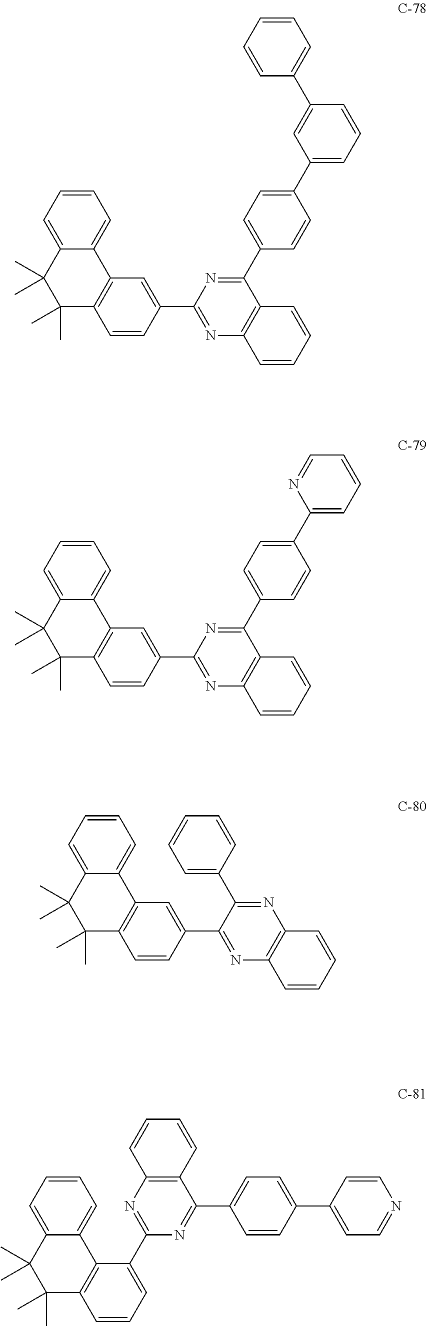 [SMILES: CC1(C)c2ccccc2-c2cc(-c3nc(-c4ccc(-c5cccc(-c6ccccc6)c5)cc4)c4ccccc4n3)ccc2C1(C)C ; CC1(C)c2ccccc2-c2cc(-c3nc(-c4ccc(-c5ccccn5)cc4)c4ccccc4n3)ccc2C1(C)C ; CC1(C)c2ccccc2-c2cc(-c3nc4ccccc4nc3-c3ccccc3)ccc2C1(C)C ; CC1(C)c2ccccc2-c2c(-c3nc(-c4ccc(-c5ccncc5)cc4)c4ccccc4n3)cccc2C1(C)C]

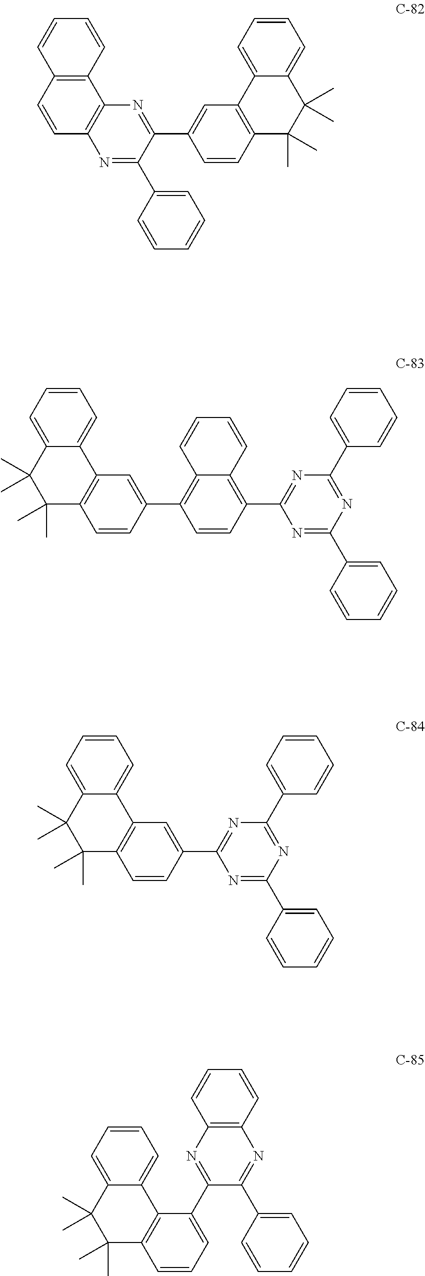

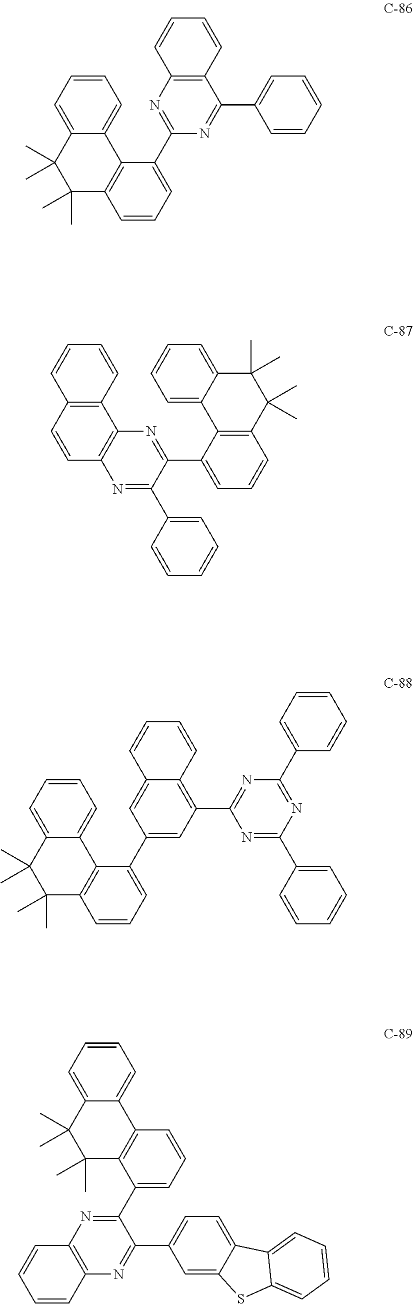

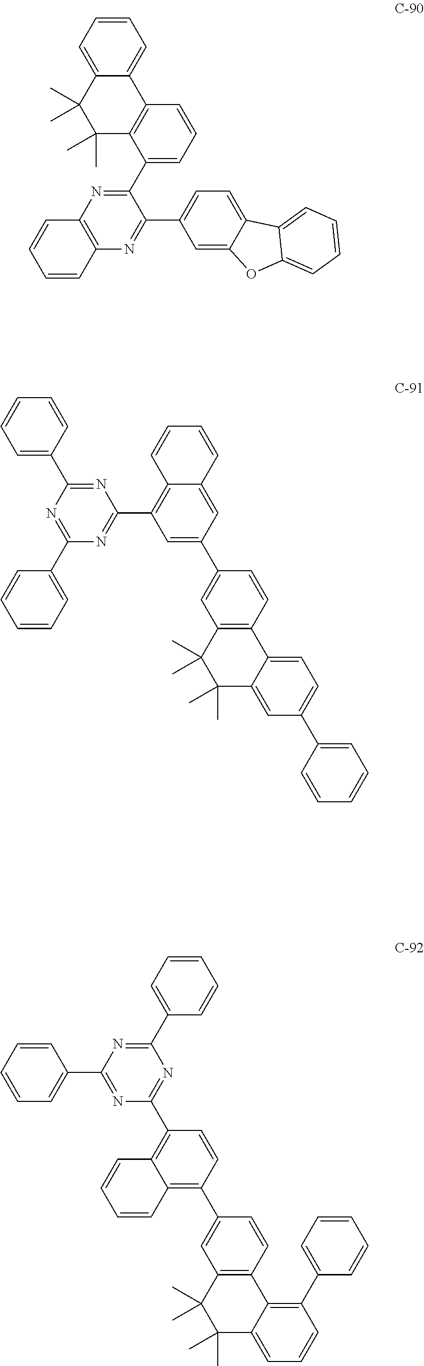 [SMILES: CC1(C)c2ccccc2-c2cccc(-c3nc4ccccc4nc3-c3ccc4c(c3)oc3ccccc34)c2C1(C)C ; CC1(C)c2cc(-c3ccccc3)ccc2-c2ccc(-c3cc(-c4nc(-c5ccccc5)nc(-c5ccccc5)n4)c4ccccc4c3)cc2C1(C)C ; CC1(C)c2cc(-c3ccc(-c4nc(-c5ccccc5)nc(-c5ccccc5)n4)c4ccccc34)ccc2-c2c(-c3ccccc3)cccc2C1(C)C]

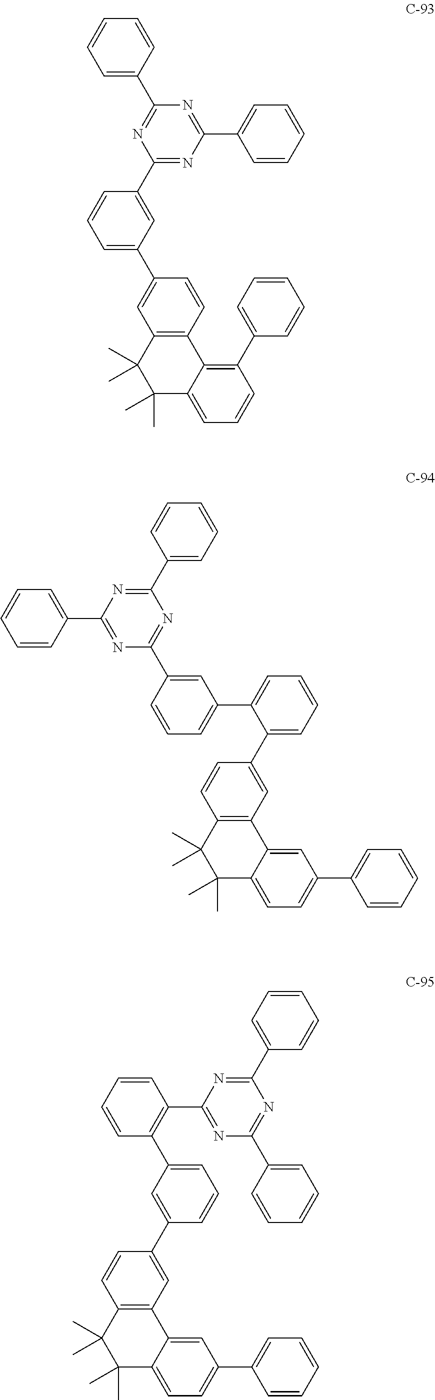 [SMILES: CC1(C)c2cc(-c3cccc(-c4nc(-c5ccccc5)nc(-c5ccccc5)n4)c3)ccc2-c2c(-c3ccccc3)cccc2C1(C)C ; CC1(C)c2ccc(-c3ccccc3)cc2-c2cc(-c3ccccc3-c3cccc(-c4nc(-c5ccccc5)nc(-c5ccccc5)n4)c3)ccc2C1(C)C ; CC1(C)c2ccc(-c3ccccc3)cc2-c2cc(-c3cccc(-c4ccccc4-c4nc(-c5ccccc5)nc(-c5ccccc5)n4)c3)ccc2C1(C)C]

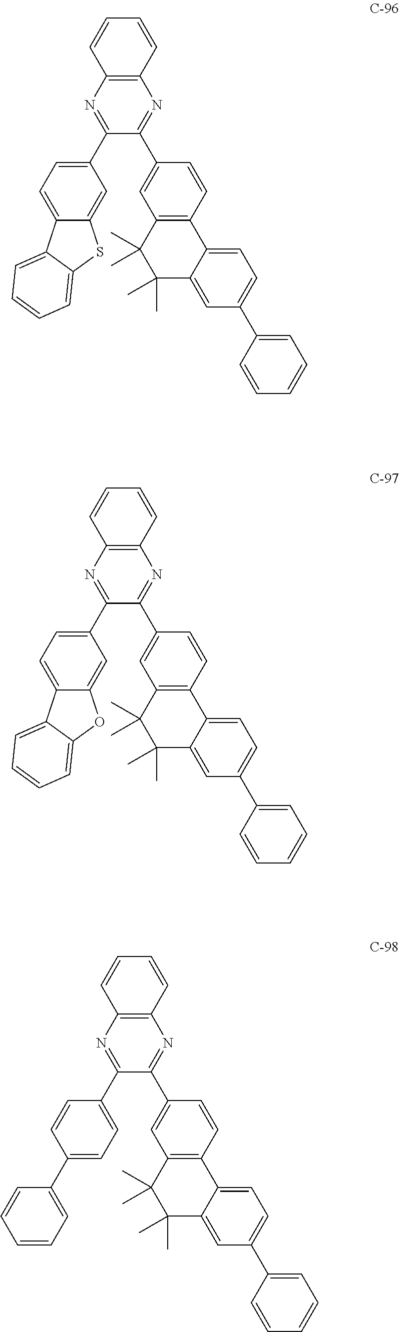 [SMILES: CC1(C)c2cc(-c3ccccc3)ccc2-c2ccc(-c3nc4ccccc4nc3-c3ccc4c(c3)sc3ccccc34)cc2C1(C)C ; CC1(C)c2cc(-c3ccccc3)ccc2-c2ccc(-c3nc4ccccc4nc3-c3ccc4c(c3)oc3ccccc34)cc2C1(C)C ; CC1(C)c2cc(-c3ccccc3)ccc2-c2ccc(-c3nc4ccccc4nc3-c3ccc(-c4ccccc4)cc3)cc2C1(C)C]

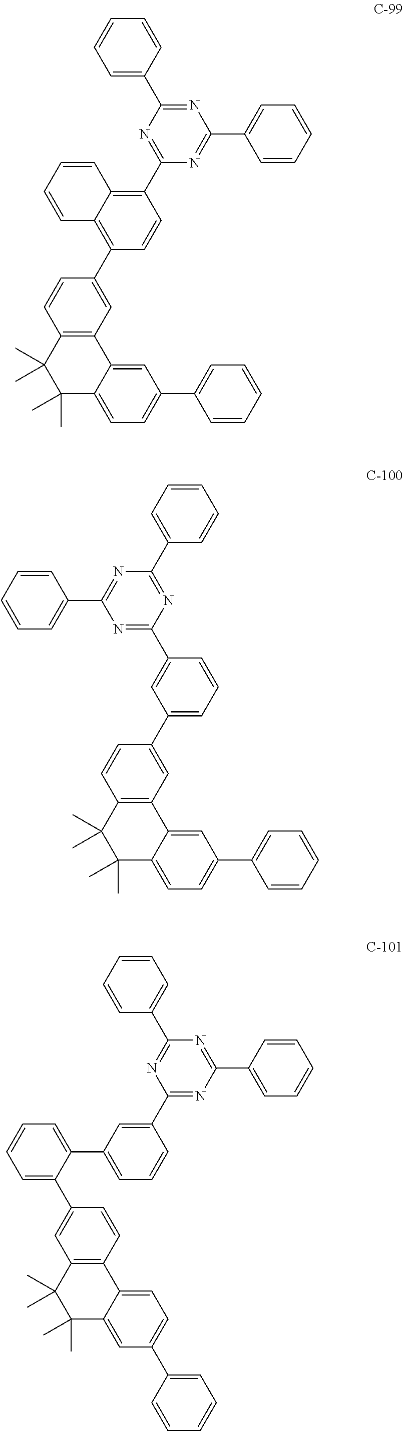 [SMILES: CC1(C)c2ccc(-c3ccccc3)cc2-c2cc(-c3ccc(-c4nc(-c5ccccc5)nc(-c5ccccc5)n4)c4ccccc34)ccc2C1(C)C ; CC1(C)c2ccc(-c3ccccc3)cc2-c2cc(-c3cccc(-c4nc(-c5ccccc5)nc(-c5ccccc5)n4)c3)ccc2C1(C)C ; CC1(C)c2cc(-c3ccccc3)ccc2-c2ccc(-c3ccccc3-c3cccc(-c4nc(-c5ccccc5)nc(-c5ccccc5)n4)c3)cc2C1(C)C]

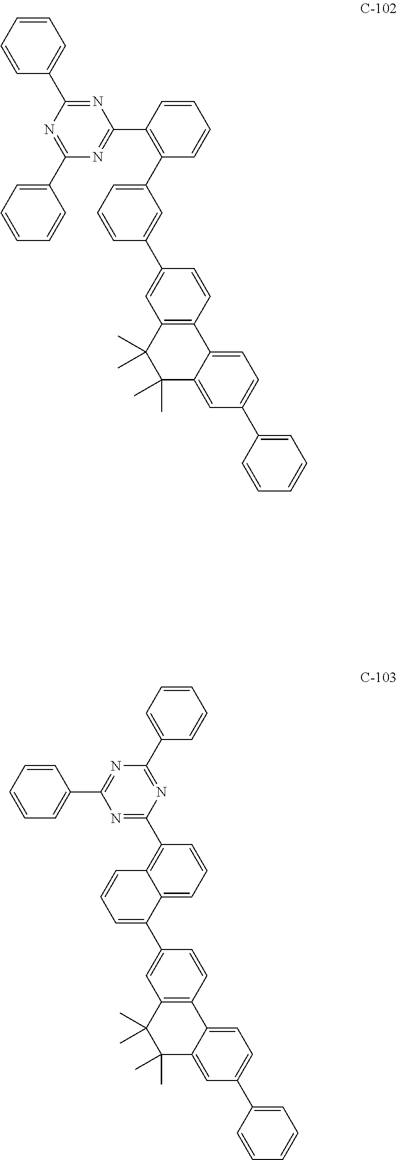 [SMILES: CC1(C)c2cc(-c3ccccc3)ccc2-c2ccc(-c3cccc(-c4ccccc4-c4nc(-c5ccccc5)nc(-c5ccccc5)n4)c3)cc2C1(C)C ; CC1(C)c2cc(-c3ccccc3)ccc2-c2ccc(-c3cccc4c(-c5nc(-c6ccccc6)nc(-c6ccccc6)n5)cccc34)cc2C1(C)C]

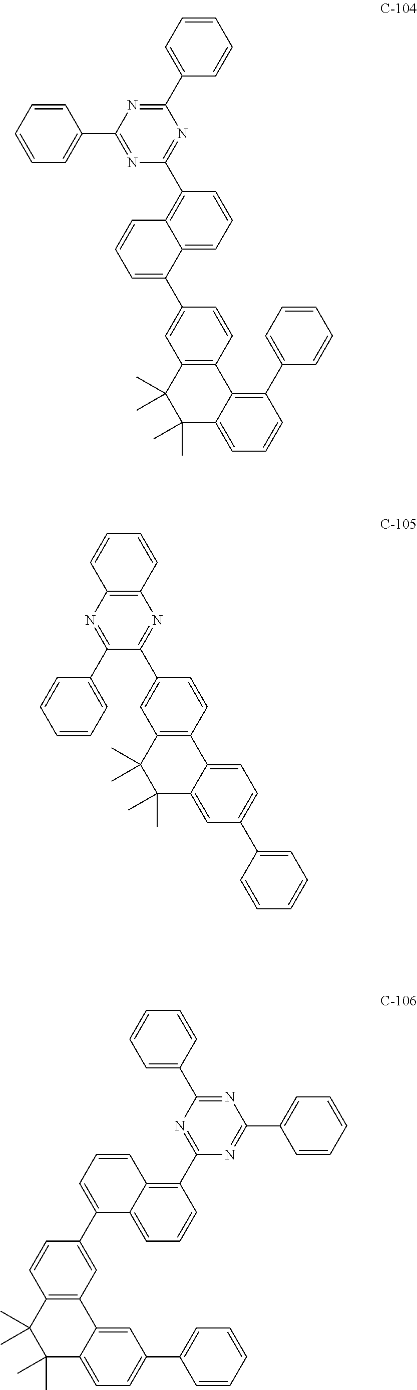 [SMILES: CC1(C)c2cc(-c3cccc4c(-c5nc(-c6ccccc6)nc(-c6ccccc6)n5)cccc34)ccc2-c2c(-c3ccccc3)cccc2C1(C)C ; CC1(C)c2cc(-c3ccccc3)ccc2-c2ccc(-c3nc4ccccc4nc3-c3ccccc3)cc2C1(C)C ; CC1(C)c2ccc(-c3ccccc3)cc2-c2cc(-c3cccc4c(-c5nc(-c6ccccc6)nc(-c6ccccc6)n5)cccc34)ccc2C1(C)C]

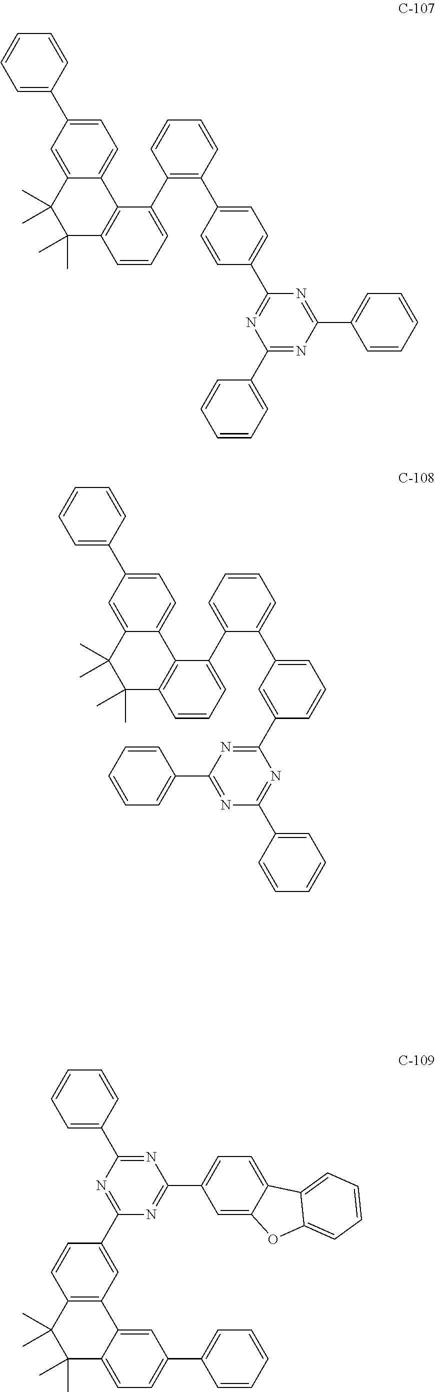 [SMILES: CC1(C)c2cc(-c3ccccc3)ccc2-c2c(-c3ccccc3-c3ccc(-c4nc(-c5ccccc5)nc(-c5ccccc5)n4)cc3)cccc2C1(C)C ; CC1(C)c2cc(-c3ccccc3)ccc2-c2c(-c3ccccc3-c3cccc(-c4nc(-c5ccccc5)nc(-c5ccccc5)n4)c3)cccc2C1(C)C ; CC1(C)c2ccc(-c3ccccc3)cc2-c2cc(-c3nc(-c4ccccc4)nc(-c4ccc5c(c4)oc4ccccc45)n3)ccc2C1(C)C]

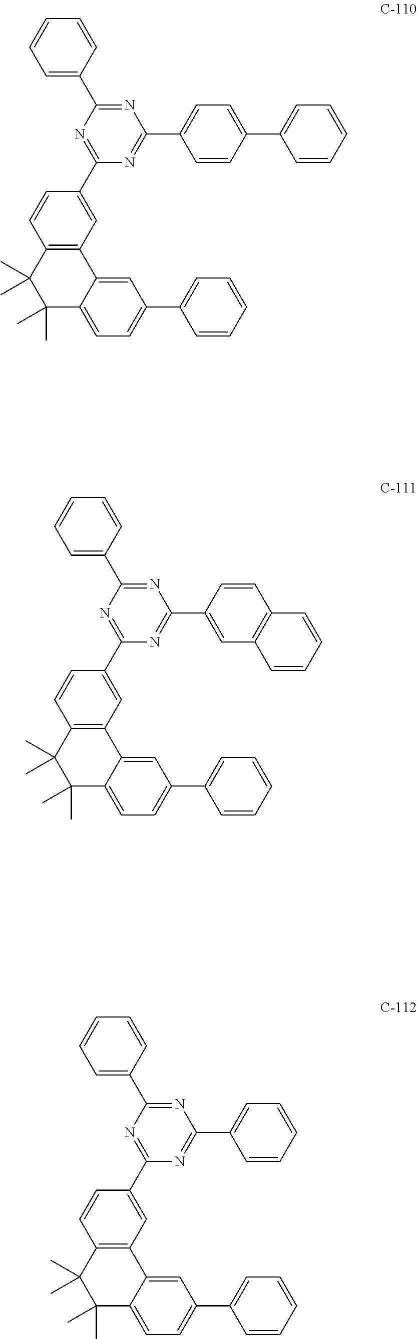 [SMILES: CC1(C)c2ccc(-c3ccccc3)cc2-c2cc(-c3nc(-c4ccccc4)nc(-c4ccc(-c5ccccc5)cc4)n3)ccc2C1(C)C ; CC1(C)c2ccc(-c3ccccc3)cc2-c2cc(-c3nc(-c4ccccc4)nc(-c4ccc5ccccc5c4)n3)ccc2C1(C)C ; CC1(C)c2ccc(-c3ccccc3)cc2-c2cc(-c3nc(-c4ccccc4)nc(-c4ccccc4)n3)ccc2C1(C)C]

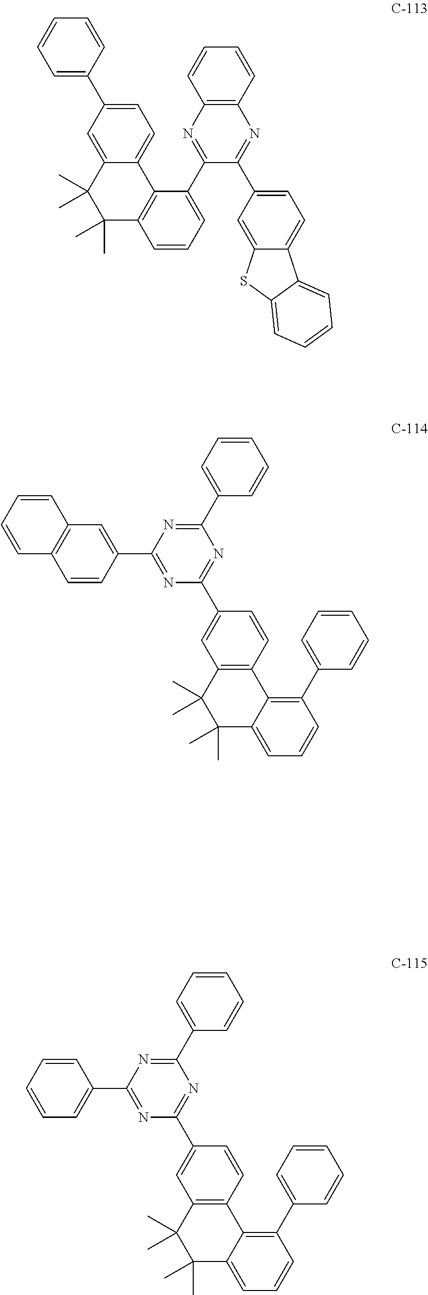 [SMILES: CC1(C)c2cc(-c3ccccc3)ccc2-c2c(-c3nc4ccccc4nc3-c3ccc4c(c3)sc3ccccc34)cccc2C1(C)C ; CC1(C)c2cc(-c3nc(-c4ccccc4)nc(-c4ccc5ccccc5c4)n3)ccc2-c2c(-c3ccccc3)cccc2C1(C)C ; CC1(C)c2cc(-c3nc(-c4ccccc4)nc(-c4ccccc4)n3)ccc2-c2c(-c3ccccc3)cccc2C1(C)C]

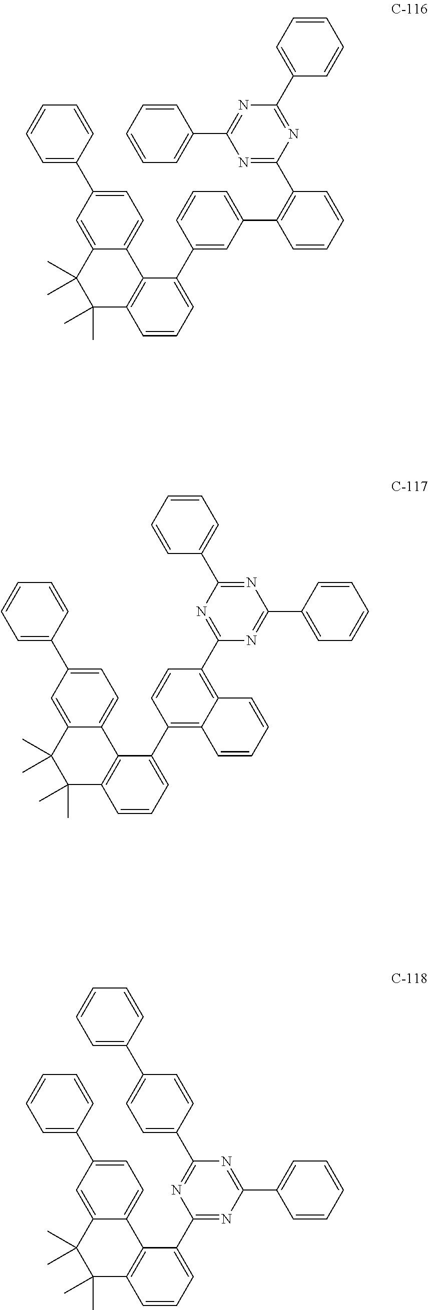 [SMILES: CC1(C)c2cc(-c3ccccc3)ccc2-c2c(-c3cccc(-c4ccccc4-c4nc(-c5ccccc5)nc(-c5ccccc5)n4)c3)cccc2C1(C)C ; CC1(C)c2cc(-c3ccccc3)ccc2-c2c(-c3ccc(-c4nc(-c5ccccc5)nc(-c5ccccc5)n4)c4ccccc34)cccc2C1(C)C ; CC1(C)c2cc(-c3ccccc3)ccc2-c2c(-c3nc(-c4ccccc4)nc(-c4ccc(-c5ccccc5)cc4)n3)cccc2C1(C)C]

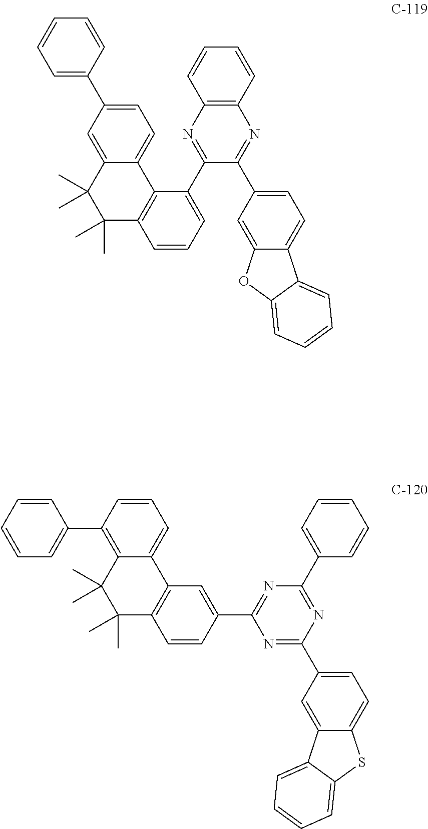 [SMILES: CC1(C)c2cc(-c3ccccc3)ccc2-c2c(-c3nc4ccccc4nc3-c3ccc4c(c3)oc3ccccc34)cccc2C1(C)C ; CC1(C)c2ccc(-c3nc(-c4ccccc4)nc(-c4ccc5sc6ccccc6c5c4)n3)cc2-c2cccc(-c3ccccc3)c2C1(C)C]

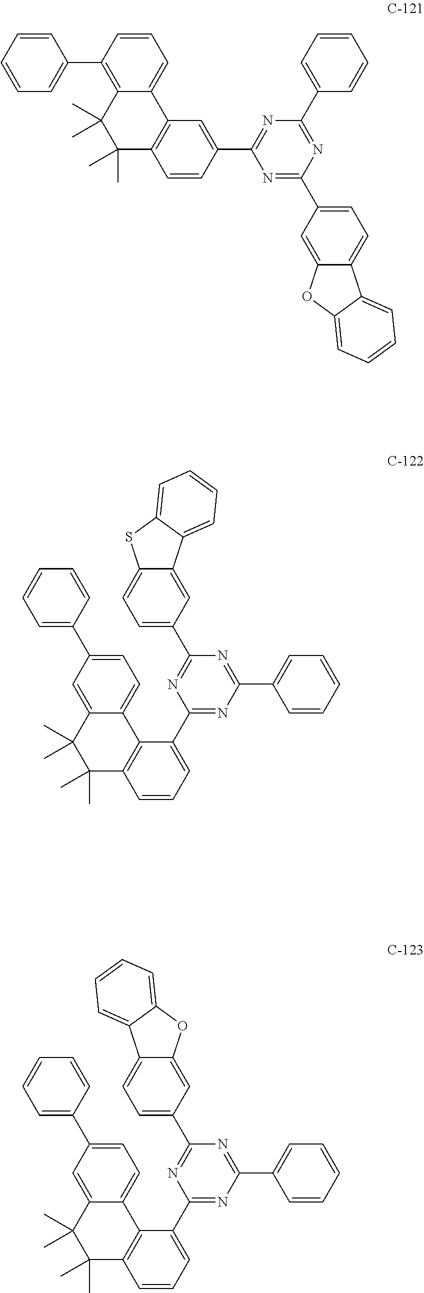 [SMILES: CC1(C)c2ccc(-c3nc(-c4ccccc4)nc(-c4ccc5c(c4)oc4ccccc45)n3)cc2-c2cccc(-c3ccccc3)c2C1(C)C ; CC1(C)c2cc(-c3ccccc3)ccc2-c2c(-c3nc(-c4ccccc4)nc(-c4ccc5sc6ccccc6c5c4)n3)cccc2C1(C)C ; CC1(C)c2cc(-c3ccccc3)ccc2-c2c(-c3nc(-c4ccccc4)nc(-c4ccc5c(c4)oc4ccccc45)n3)cccc2C1(C)C]

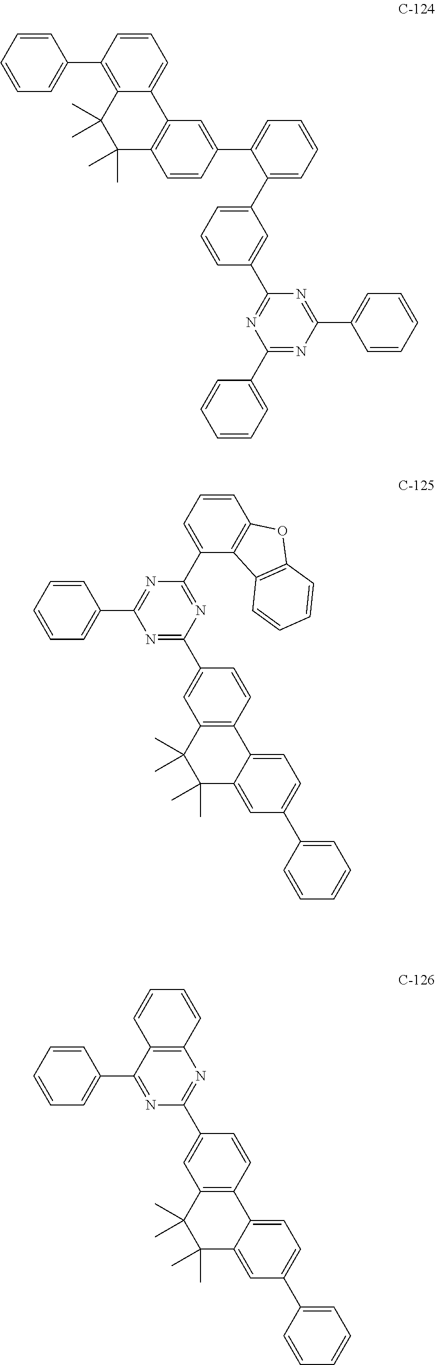 [SMILES: CC1(C)c2ccc(-c3ccccc3-c3cccc(-c4nc(-c5ccccc5)nc(-c5ccccc5)n4)c3)cc2-c2cccc(-c3ccccc3)c2C1(C)C ; CC1(C)c2cc(-c3ccccc3)ccc2-c2ccc(-c3nc(-c4ccccc4)nc(-c4cccc5oc6ccccc6c45)n3)cc2C1(C)C ; CC1(C)c2cc(-c3ccccc3)ccc2-c2ccc(-c3nc(-c4ccccc4)c4ccccc4n3)cc2C1(C)C]

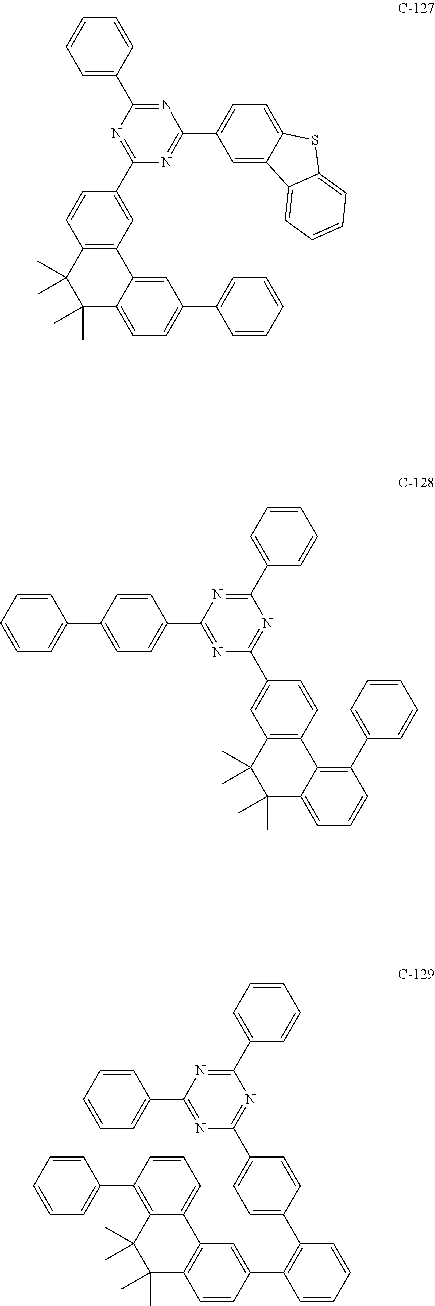 [SMILES: CC1(C)c2ccc(-c3ccccc3)cc2-c2cc(-c3nc(-c4ccccc4)nc(-c4ccc5sc6ccccc6c5c4)n3)ccc2C1(C)C ; CC1(C)c2cc(-c3nc(-c4ccccc4)nc(-c4ccc(-c5ccccc5)cc4)n3)ccc2-c2c(-c3ccccc3)cccc2C1(C)C ; CC1(C)c2ccc(-c3ccccc3-c3ccc(-c4nc(-c5ccccc5)nc(-c5ccccc5)n4)cc3)cc2-c2cccc(-c3ccccc3)c2C1(C)C]

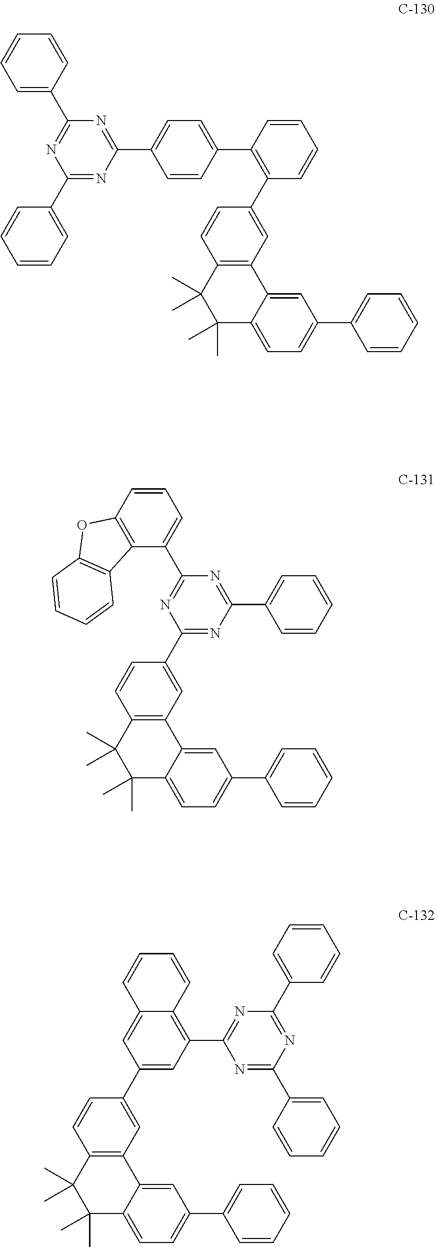 [SMILES: CC1(C)c2ccc(-c3ccccc3)cc2-c2cc(-c3ccccc3-c3ccc(-c4nc(-c5ccccc5)nc(-c5ccccc5)n4)cc3)ccc2C1(C)C ; CC1(C)c2ccc(-c3ccccc3)cc2-c2cc(-c3nc(-c4ccccc4)nc(-c4cccc5oc6ccccc6c45)n3)ccc2C1(C)C ; CC1(C)c2ccc(-c3ccccc3)cc2-c2cc(-c3cc(-c4nc(-c5ccccc5)nc(-c5ccccc5)n4)c4ccccc4c3)ccc2C1(C)C]

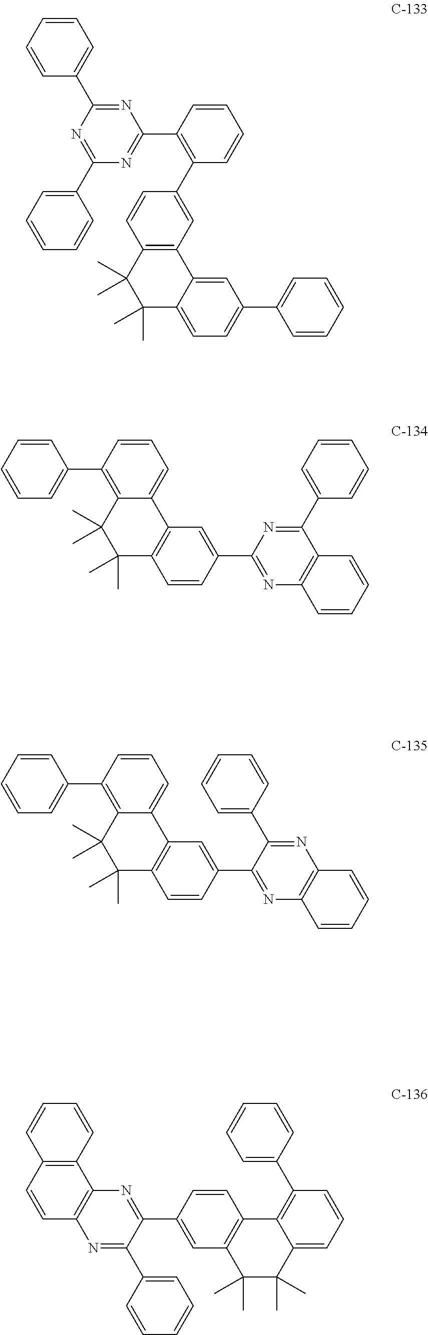 [SMILES: CC1(C)c2ccc(-c3ccccc3)cc2-c2cc(-c3ccccc3-c3nc(-c4ccccc4)nc(-c4ccccc4)n3)ccc2C1(C)C ; CC1(C)c2ccc(-c3nc(-c4ccccc4)c4ccccc4n3)cc2-c2cccc(-c3ccccc3)c2C1(C)C ; CC1(C)c2ccc(-c3nc4ccccc4nc3-c3ccccc3)cc2-c2cccc(-c3ccccc3)c2C1(C)C ; CC1(C)c2cc(-c3nc4c(ccc5ccccc54)nc3-c3ccccc3)ccc2-c2c(-c3ccccc3)cccc2C1(C)C]

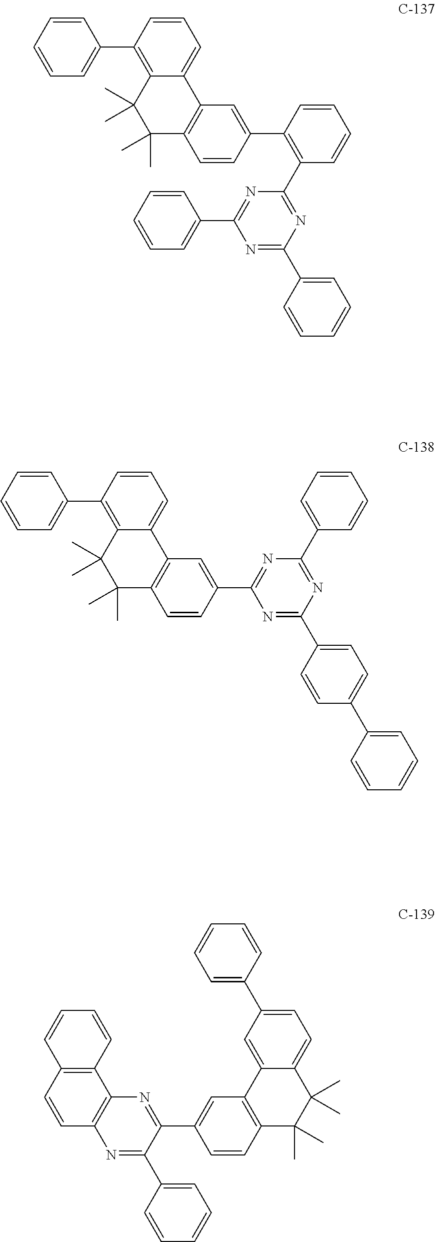 [SMILES: CC1(C)c2ccc(-c3ccccc3-c3nc(-c4ccccc4)nc(-c4ccccc4)n3)cc2-c2cccc(-c3ccccc3)c2C1(C)C ; CC1(C)c2ccc(-c3nc(-c4ccccc4)nc(-c4ccc(-c5ccccc5)cc4)n3)cc2-c2cccc(-c3ccccc3)c2C1(C)C ; CC1(C)c2ccc(-c3ccccc3)cc2-c2cc(-c3nc4c(ccc5ccccc54)nc3-c3ccccc3)ccc2C1(C)C]

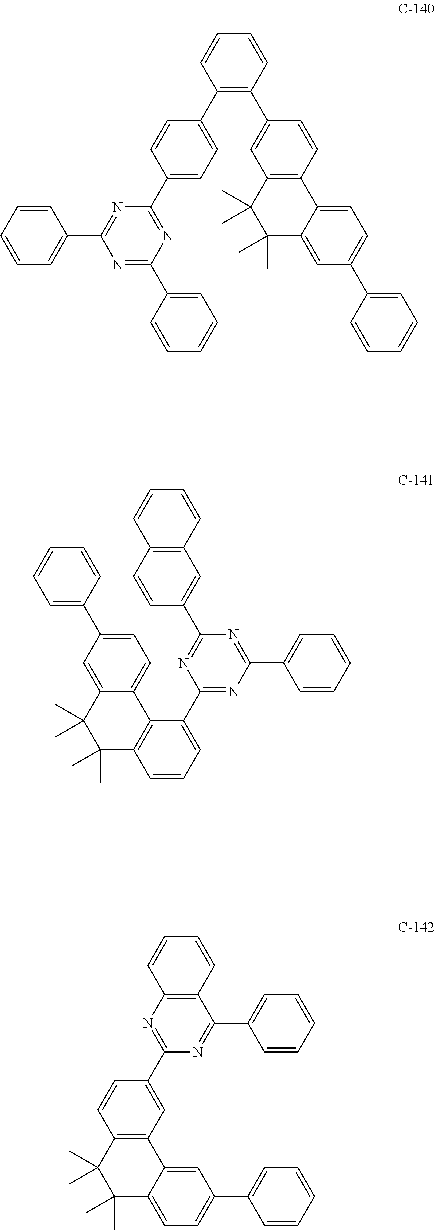 [SMILES: CC1(C)c2cc(-c3ccccc3)ccc2-c2ccc(-c3ccccc3-c3ccc(-c4nc(-c5ccccc5)nc(-c5ccccc5)n4)cc3)cc2C1(C)C ; CC1(C)c2cc(-c3ccccc3)ccc2-c2c(-c3nc(-c4ccccc4)nc(-c4ccc5ccccc5c4)n3)cccc2C1(C)C ; CC1(C)c2ccc(-c3ccccc3)cc2-c2cc(-c3nc(-c4ccccc4)c4ccccc4n3)ccc2C1(C)C]

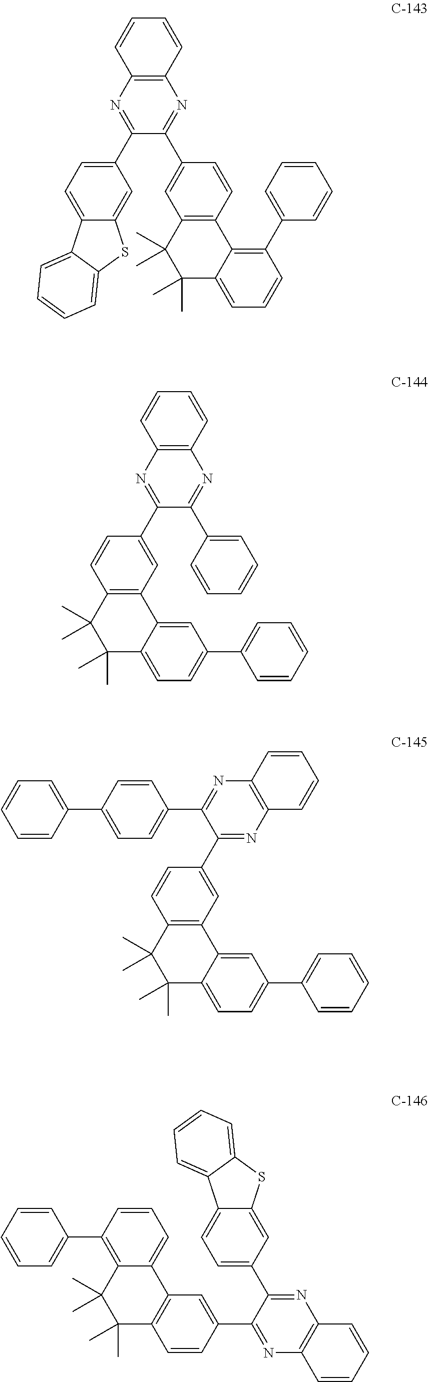 [SMILES: CC1(C)c2cc(-c3nc4ccccc4nc3-c3ccc4c(c3)sc3ccccc34)ccc2-c2c(-c3ccccc3)cccc2C1(C)C ; CC1(C)c2ccc(-c3ccccc3)cc2-c2cc(-c3nc4ccccc4nc3-c3ccccc3)ccc2C1(C)C ; CC1(C)c2ccc(-c3ccccc3)cc2-c2cc(-c3nc4ccccc4nc3-c3ccc(-c4ccccc4)cc3)ccc2C1(C)C ; CC1(C)c2ccc(-c3nc4ccccc4nc3-c3ccc4c(c3)sc3ccccc34)cc2-c2cccc(-c3ccccc3)c2C1(C)C]

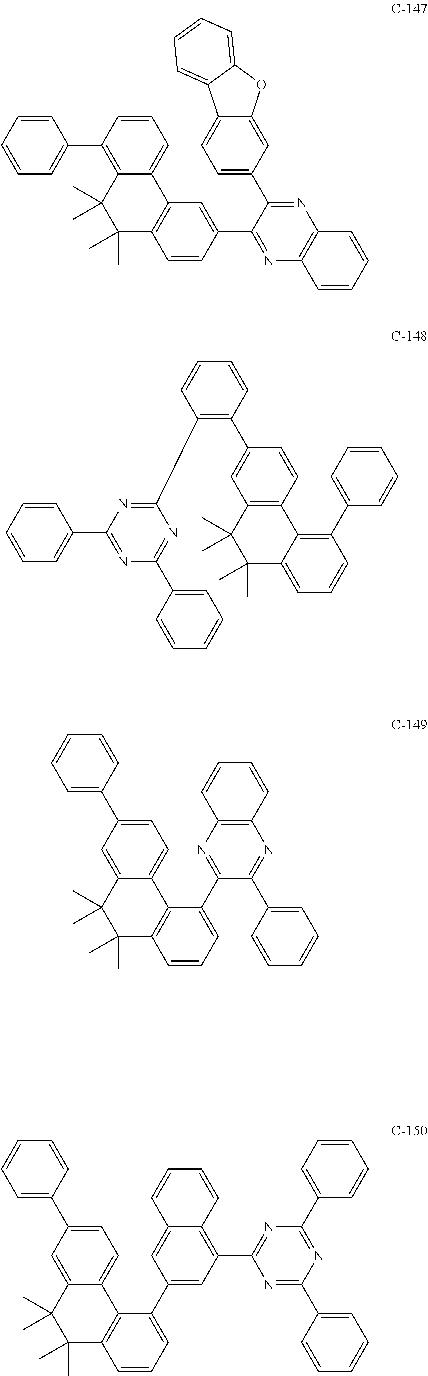 [SMILES: CC1(C)c2ccc(-c3nc4ccccc4nc3-c3ccc4c(c3)oc3ccccc34)cc2-c2cccc(-c3ccccc3)c2C1(C)C ; CC1(C)c2cc(-c3ccccc3-c3nc(-c4ccccc4)nc(-c4ccccc4)n3)ccc2-c2c(-c3ccccc3)cccc2C1(C)C ; CC1(C)c2cc(-c3ccccc3)ccc2-c2c(-c3nc4ccccc4nc3-c3ccccc3)cccc2C1(C)C ; CC1(C)c2cc(-c3ccccc3)ccc2-c2c(-c3cc(-c4nc(-c5ccccc5)nc(-c5ccccc5)n4)c4ccccc4c3)cccc2C1(C)C]

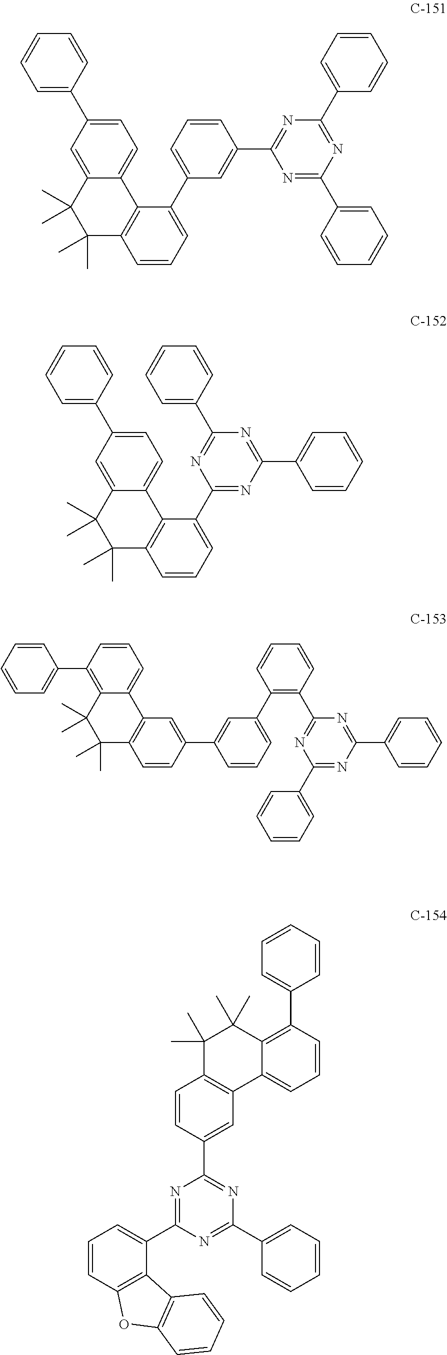 [SMILES: CC1(C)c2cc(-c3ccccc3)ccc2-c2c(-c3cccc(-c4nc(-c5ccccc5)nc(-c5ccccc5)n4)c3)cccc2C1(C)C ; CC1(C)c2cc(-c3ccccc3)ccc2-c2c(-c3nc(-c4ccccc4)nc(-c4ccccc4)n3)cccc2C1(C)C ; CC1(C)c2ccc(-c3cccc(-c4ccccc4-c4nc(-c5ccccc5)nc(-c5ccccc5)n4)c3)cc2-c2cccc(-c3ccccc3)c2C1(C)C ; CC1(C)c2ccc(-c3nc(-c4ccccc4)nc(-c4cccc5oc6ccccc6c45)n3)cc2-c2cccc(-c3ccccc3)c2C1(C)C]

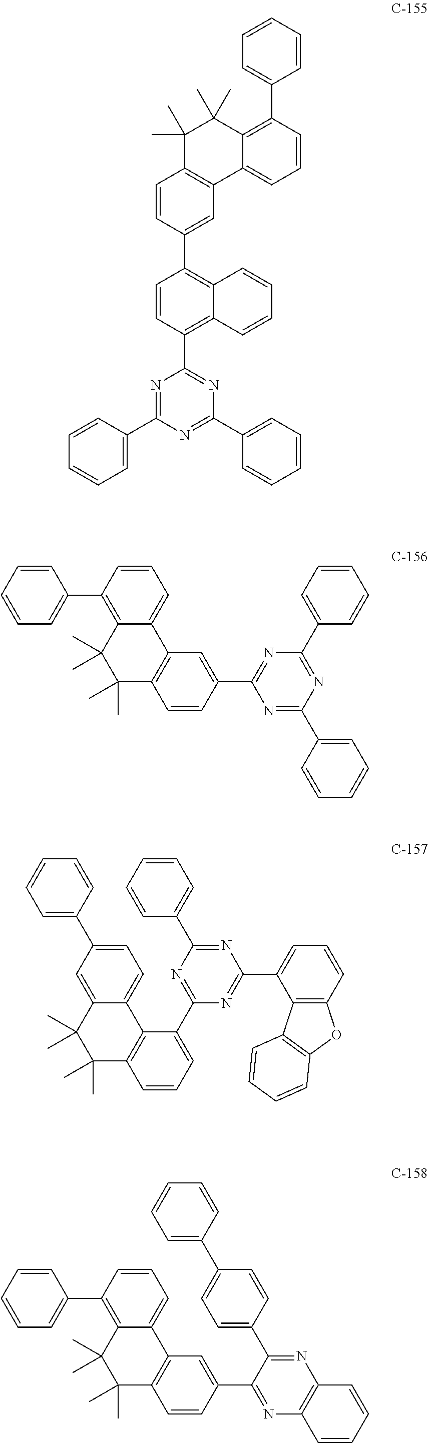 [SMILES: CC1(C)c2ccc(-c3ccc(-c4nc(-c5ccccc5)nc(-c5ccccc5)n4)c4ccccc34)cc2-c2cccc(-c3ccccc3)c2C1(C)C ; CC1(C)c2ccc(-c3nc(-c4ccccc4)nc(-c4ccccc4)n3)cc2-c2cccc(-c3ccccc3)c2C1(C)C ; CC1(C)c2cc(-c3ccccc3)ccc2-c2c(-c3nc(-c4ccccc4)nc(-c4cccc5oc6ccccc6c45)n3)cccc2C1(C)C ; CC1(C)c2ccc(-c3nc4ccccc4nc3-c3ccc(-c4ccccc4)cc3)cc2-c2cccc(-c3ccccc3)c2C1(C)C]

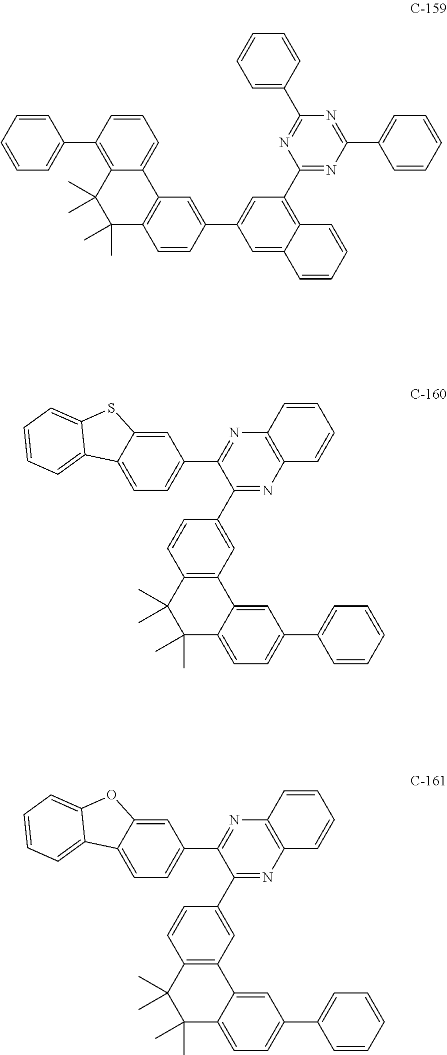 [SMILES: CC1(C)c2ccc(-c3cc(-c4nc(-c5ccccc5)nc(-c5ccccc5)n4)c4ccccc4c3)cc2-c2cccc(-c3ccccc3)c2C1(C)C ; CC1(C)c2ccc(-c3ccccc3)cc2-c2cc(-c3nc4ccccc4nc3-c3ccc4c(c3)sc3ccccc34)ccc2C1(C)C ; CC1(C)c2ccc(-c3ccccc3)cc2-c2cc(-c3nc4ccccc4nc3-c3ccc4c(c3)oc3ccccc34)ccc2C1(C)C]

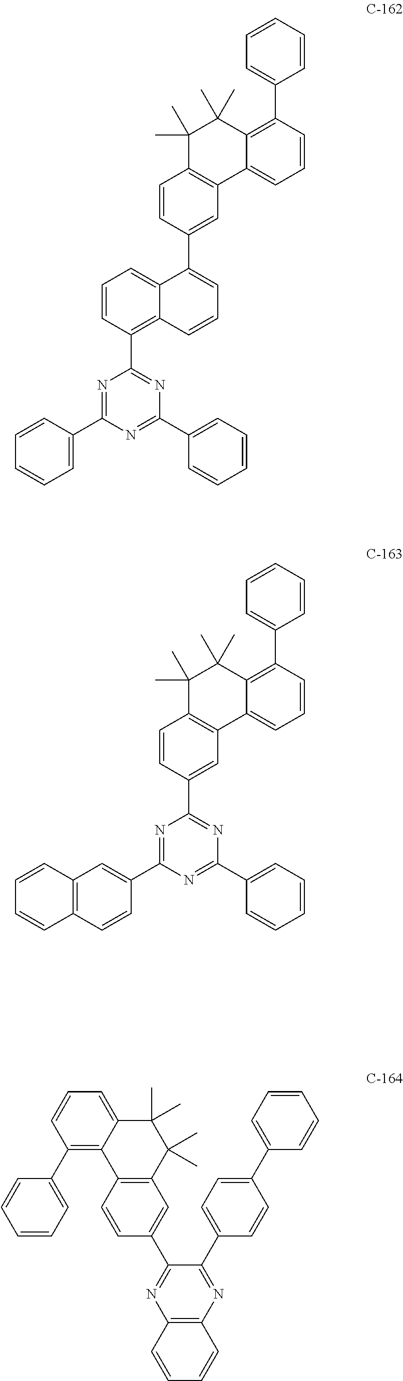 [SMILES: CC1(C)c2ccc(-c3cccc4c(-c5nc(-c6ccccc6)nc(-c6ccccc6)n5)cccc34)cc2-c2cccc(-c3ccccc3)c2C1(C)C ; CC1(C)c2ccc(-c3nc(-c4ccccc4)nc(-c4ccc5ccccc5c4)n3)cc2-c2cccc(-c3ccccc3)c2C1(C)C ; CC1(C)c2cc(-c3nc4ccccc4nc3-c3ccc(-c4ccccc4)cc3)ccc2-c2c(-c3ccccc3)cccc2C1(C)C]

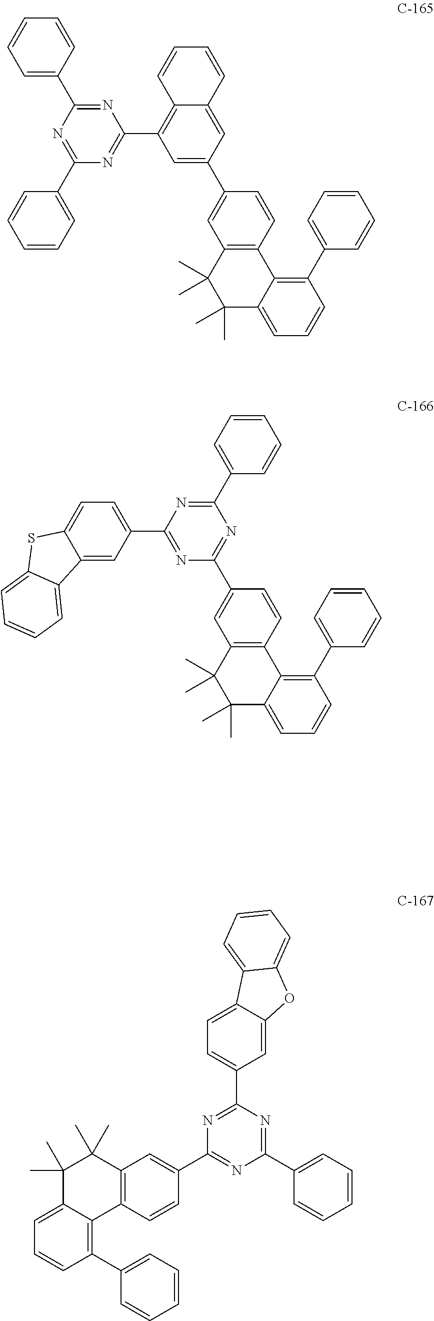 [SMILES: CC1(C)c2cc(-c3cc(-c4nc(-c5ccccc5)nc(-c5ccccc5)n4)c4ccccc4c3)ccc2-c2c(-c3ccccc3)cccc2C1(C)C ; CC1(C)c2cc(-c3nc(-c4ccccc4)nc(-c4ccc5sc6ccccc6c5c4)n3)ccc2-c2c(-c3ccccc3)cccc2C1(C)C ; CC1(C)c2cc(-c3nc(-c4ccccc4)nc(-c4ccc5c(c4)oc4ccccc45)n3)ccc2-c2c(-c3ccccc3)cccc2C1(C)C]

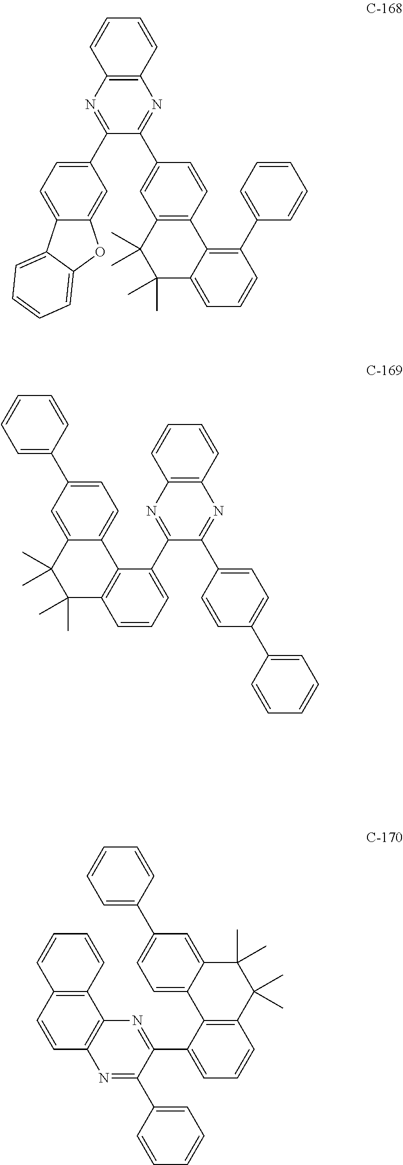 [SMILES: CC1(C)c2cc(-c3nc4ccccc4nc3-c3ccc4c(c3)oc3ccccc34)ccc2-c2c(-c3ccccc3)cccc2C1(C)C ; CC1(C)c2cc(-c3ccccc3)ccc2-c2c(-c3nc4ccccc4nc3-c3ccc(-c4ccccc4)cc3)cccc2C1(C)C ; CC1(C)c2cc(-c3ccccc3)ccc2-c2c(-c3nc4c(ccc5ccccc54)nc3-c3ccccc3)cccc2C1(C)C]

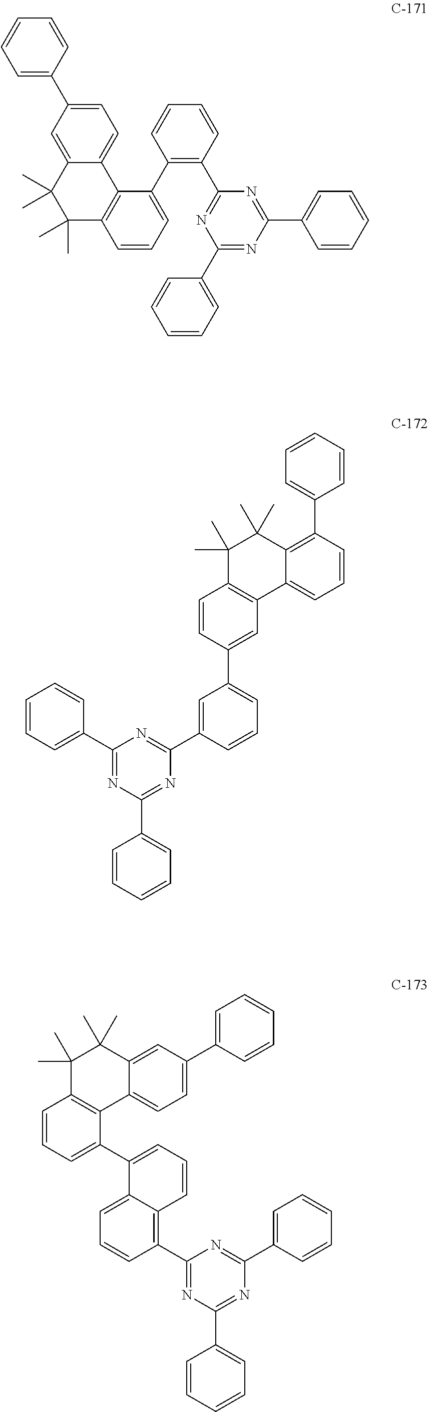 [SMILES: CC1(C)c2cc(-c3ccccc3)ccc2-c2c(-c3ccccc3-c3nc(-c4ccccc4)nc(-c4ccccc4)n3)cccc2C1(C)C ; CC1(C)c2ccc(-c3cccc(-c4nc(-c5ccccc5)nc(-c5ccccc5)n4)c3)cc2-c2cccc(-c3ccccc3)c2C1(C)C ; CC1(C)c2cc(-c3ccccc3)ccc2-c2c(-c3cccc4c(-c5nc(-c6ccccc6)nc(-c6ccccc6)n5)cccc34)cccc2C1(C)C]

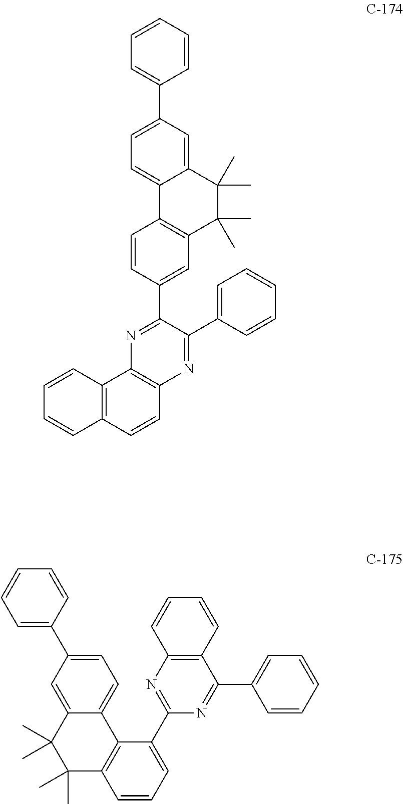 [SMILES: CC1(C)c2cc(-c3ccccc3)ccc2-c2ccc(-c3nc4c(ccc5ccccc54)nc3-c3ccccc3)cc2C1(C)C ; CC1(C)c2cc(-c3ccccc3)ccc2-c2c(-c3nc(-c4ccccc4)c4ccccc4n3)cccc2C1(C)C]

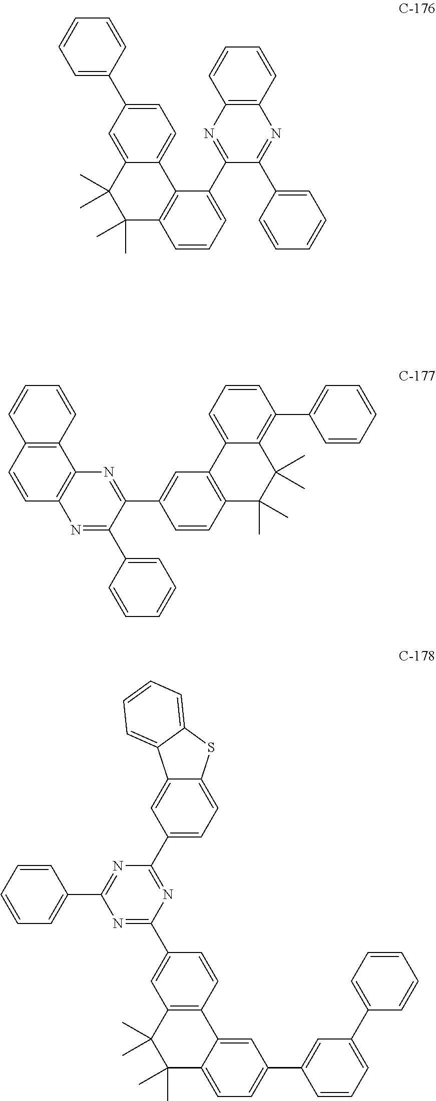 [SMILES: CC1(C)c2cc(-c3ccccc3)ccc2-c2c(-c3nc4ccccc4nc3-c3ccccc3)cccc2C1(C)C ; CC1(C)c2ccc(-c3nc4c(ccc5ccccc54)nc3-c3ccccc3)cc2-c2cccc(-c3ccccc3)c2C1(C)C ; CC1(C)c2ccc(-c3cccc(-c4ccccc4)c3)cc2-c2ccc(-c3nc(-c4ccccc4)nc(-c4ccc5sc6ccccc6c5c4)n3)cc2C1(C)C]

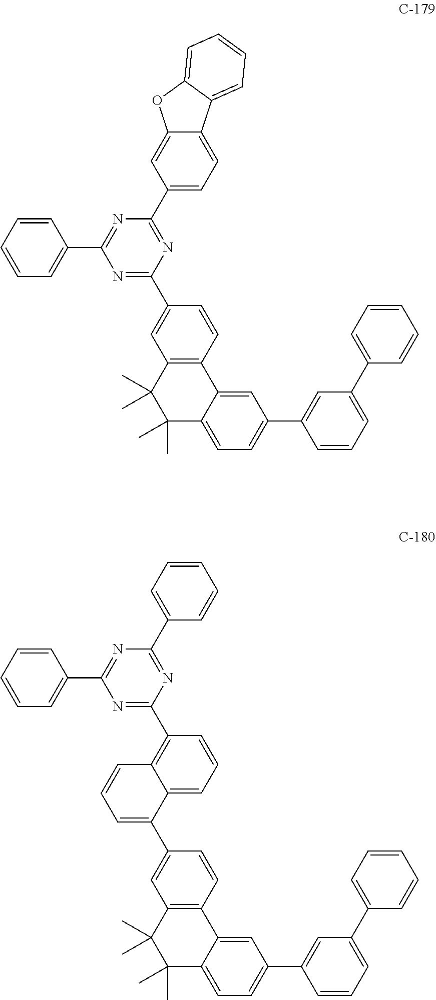 [SMILES: CC1(C)c2ccc(-c3cccc(-c4ccccc4)c3)cc2-c2ccc(-c3nc(-c4ccccc4)nc(-c4ccc5c(c4)oc4ccccc45)n3)cc2C1(C)C ; CC1(C)c2ccc(-c3cccc(-c4ccccc4)c3)cc2-c2ccc(-c3cccc4c(-c5nc(-c6ccccc6)nc(-c6ccccc6)n5)cccc34)cc2C1(C)C]

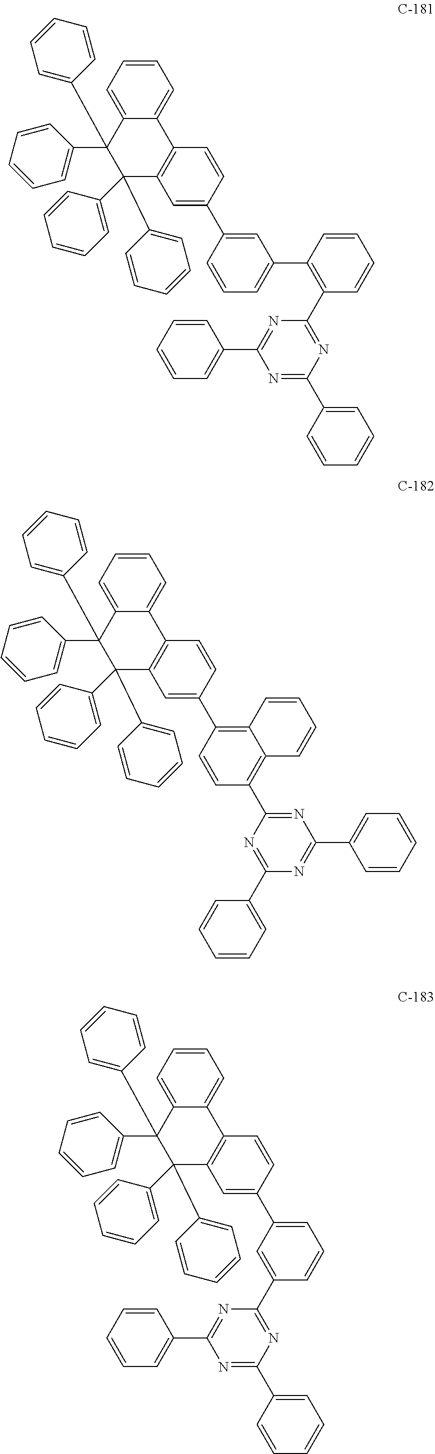 [SMILES: c1ccc(-c2nc(-c3ccccc3)nc(-c3ccccc3-c3cccc(-c4ccc5c(c4)C(c4ccccc4)(c4ccccc4)C(c4ccccc4)(c4ccccc4)c4ccccc4-5)c3)n2)cc1 ; c1ccc(-c2nc(-c3ccccc3)nc(-c3ccc(-c4ccc5c(c4)C(c4ccccc4)(c4ccccc4)C(c4ccccc4)(c4ccccc4)c4ccccc4-5)c4ccccc34)n2)cc1 ; c1ccc(-c2nc(-c3ccccc3)nc(-c3cccc(-c4ccc5c(c4)C(c4ccccc4)(c4ccccc4)C(c4ccccc4)(c4ccccc4)c4ccccc4-5)c3)n2)cc1]

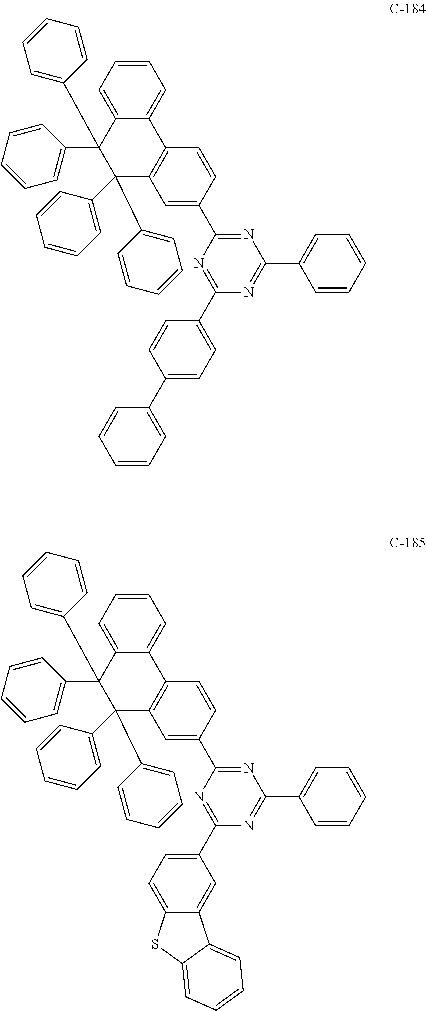 [SMILES: c1ccc(-c2ccc(-c3nc(-c4ccccc4)nc(-c4ccc5c(c4)C(c4ccccc4)(c4ccccc4)C(c4ccccc4)(c4ccccc4)c4ccccc4-5)n3)cc2)cc1 ; c1ccc(-c2nc(-c3ccc4c(c3)C(c3ccccc3)(c3ccccc3)C(c3ccccc3)(c3ccccc3)c3ccccc3-4)nc(-c3ccc4sc5ccccc5c4c3)n2)cc1]

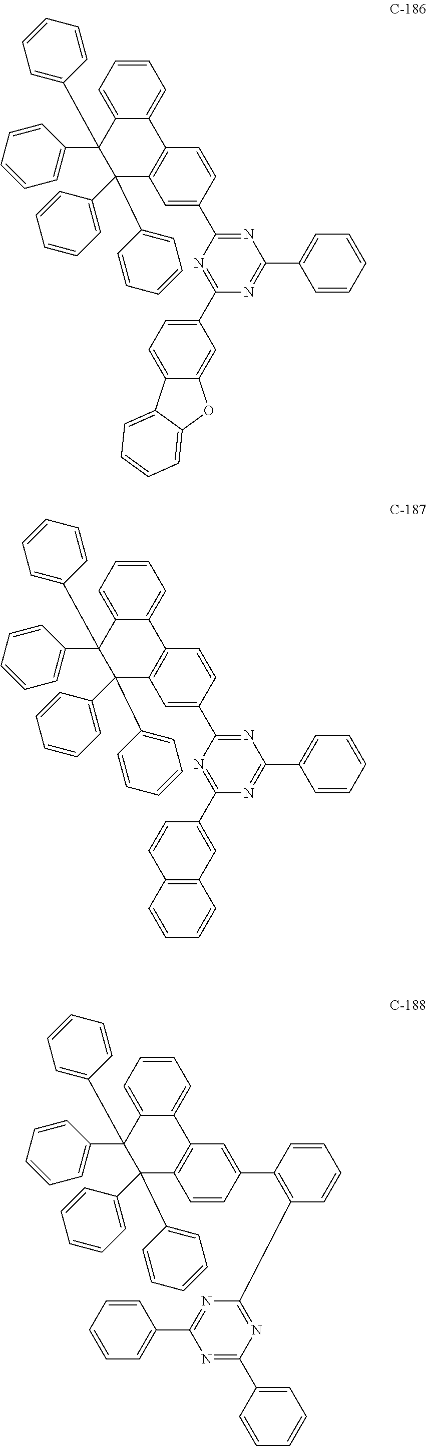 [SMILES: c1ccc(-c2nc(-c3ccc4c(c3)C(c3ccccc3)(c3ccccc3)C(c3ccccc3)(c3ccccc3)c3ccccc3-4)nc(-c3ccc4c(c3)oc3ccccc34)n2)cc1 ; c1ccc(-c2nc(-c3ccc4c(c3)C(c3ccccc3)(c3ccccc3)C(c3ccccc3)(c3ccccc3)c3ccccc3-4)nc(-c3ccc4ccccc4c3)n2)cc1 ; c1ccc(-c2nc(-c3ccccc3)nc(-c3ccccc3-c3ccc4c(c3)-c3ccccc3C(c3ccccc3)(c3ccccc3)C4(c3ccccc3)c3ccccc3)n2)cc1]

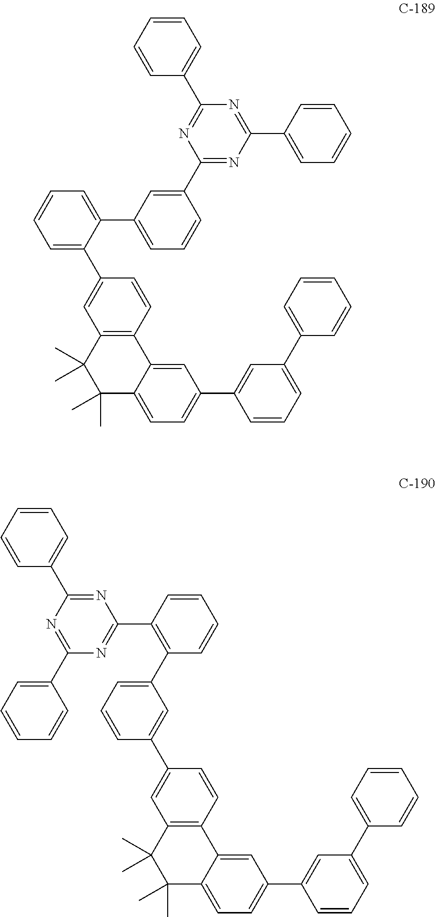 [SMILES: CC1(C)c2ccc(-c3cccc(-c4ccccc4)c3)cc2-c2ccc(-c3ccccc3-c3cccc(-c4nc(-c5ccccc5)nc(-c5ccccc5)n4)c3)cc2C1(C)C ; CC1(C)c2ccc(-c3cccc(-c4ccccc4)c3)cc2-c2ccc(-c3cccc(-c4ccccc4-c4nc(-c5ccccc5)nc(-c5ccccc5)n4)c3)cc2C1(C)C]

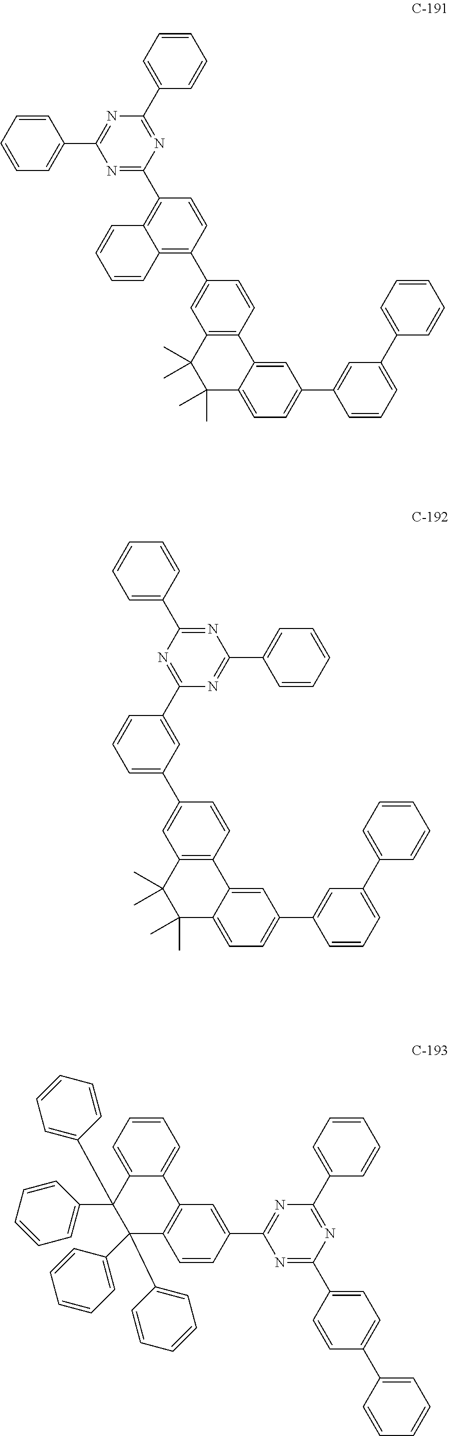

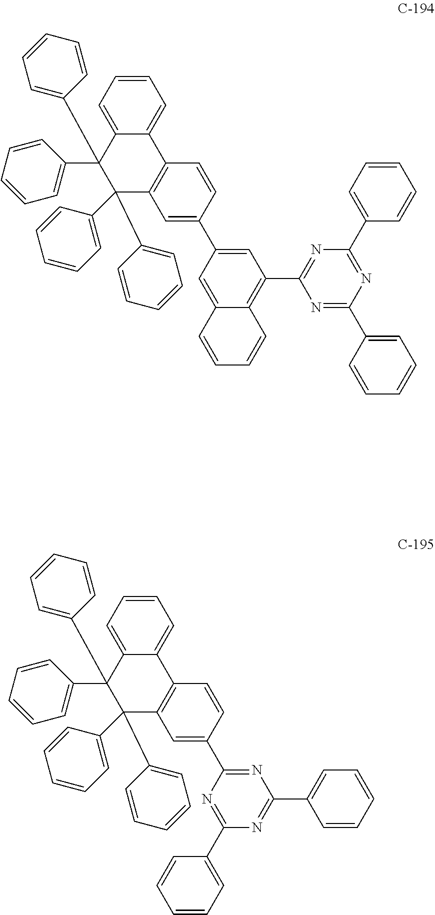 [SMILES: c1ccc(-c2nc(-c3ccccc3)nc(-c3cc(-c4ccc5c(c4)C(c4ccccc4)(c4ccccc4)C(c4ccccc4)(c4ccccc4)c4ccccc4-5)cc4ccccc34)n2)cc1 ; c1ccc(-c2nc(-c3ccccc3)nc(-c3ccc4c(c3)C(c3ccccc3)(c3ccccc3)C(c3ccccc3)(c3ccccc3)c3ccccc3-4)n2)cc1]

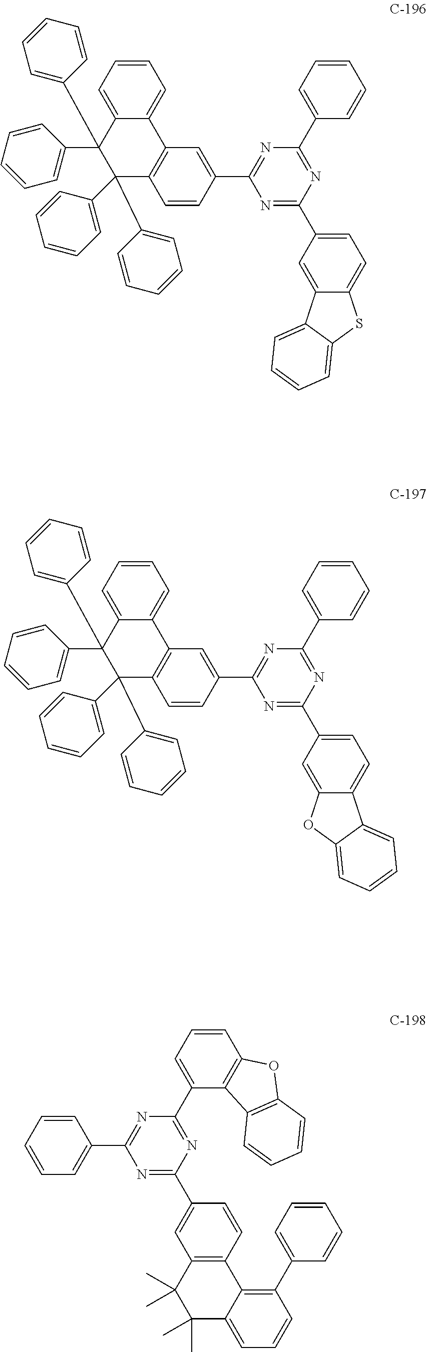 [SMILES: c1ccc(-c2nc(-c3ccc4c(c3)-c3ccccc3C(c3ccccc3)(c3ccccc3)C4(c3ccccc3)c3ccccc3)nc(-c3ccc4sc5ccccc5c4c3)n2)cc1 ; c1ccc(-c2nc(-c3ccc4c(c3)-c3ccccc3C(c3ccccc3)(c3ccccc3)C4(c3ccccc3)c3ccccc3)nc(-c3ccc4c(c3)oc3ccccc34)n2)cc1 ; CC1(C)c2cc(-c3nc(-c4ccccc4)nc(-c4cccc5oc6ccccc6c45)n3)ccc2-c2c(-c3ccccc3)cccc2C1(C)C]

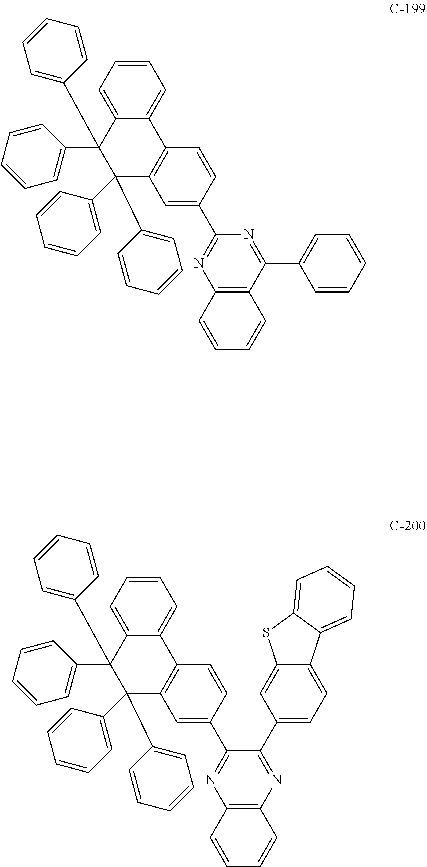 [SMILES: c1ccc(-c2nc(-c3ccc4c(c3)C(c3ccccc3)(c3ccccc3)C(c3ccccc3)(c3ccccc3)c3ccccc3-4)nc3ccccc23)cc1 ; c1ccc(C2(c3ccccc3)c3ccccc3-c3ccc(-c4nc5ccccc5nc4-c4ccc5c(c4)sc4ccccc45)cc3C2(c2ccccc2)c2ccccc2)cc1]

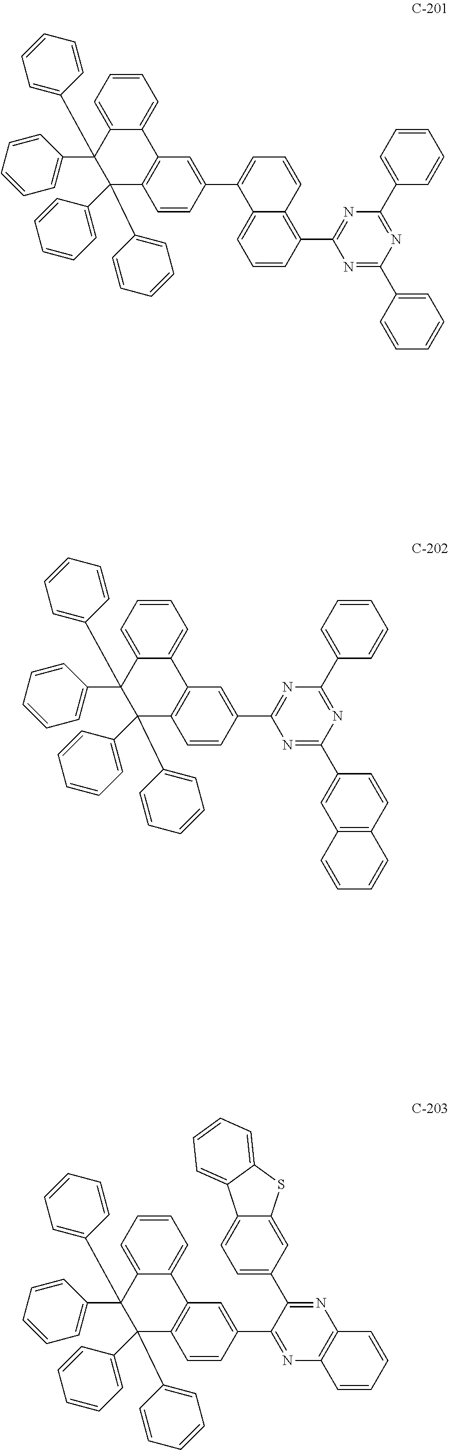 [SMILES: c1ccc(-c2nc(-c3ccccc3)nc(-c3cccc4c(-c5ccc6c(c5)-c5ccccc5C(c5ccccc5)(c5ccccc5)C6(c5ccccc5)c5ccccc5)cccc34)n2)cc1 ; c1ccc(-c2nc(-c3ccc4c(c3)-c3ccccc3C(c3ccccc3)(c3ccccc3)C4(c3ccccc3)c3ccccc3)nc(-c3ccc4ccccc4c3)n2)cc1 ; c1ccc(C2(c3ccccc3)c3ccccc3-c3cc(-c4nc5ccccc5nc4-c4ccc5c(c4)sc4ccccc45)ccc3C2(c2ccccc2)c2ccccc2)cc1]

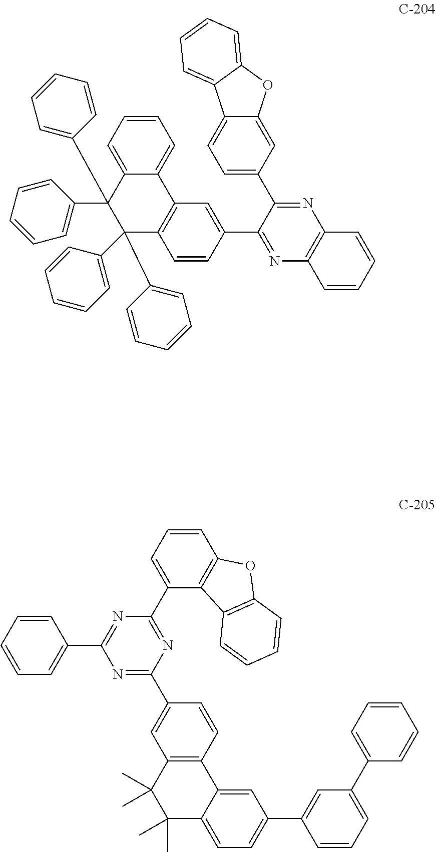 [SMILES: c1ccc(C2(c3ccccc3)c3ccccc3-c3cc(-c4nc5ccccc5nc4-c4ccc5c(c4)oc4ccccc45)ccc3C2(c2ccccc2)c2ccccc2)cc1 ; CC1(C)c2ccc(-c3cccc(-c4ccccc4)c3)cc2-c2ccc(-c3nc(-c4ccccc4)nc(-c4cccc5oc6ccccc6c45)n3)cc2C1(C)C]

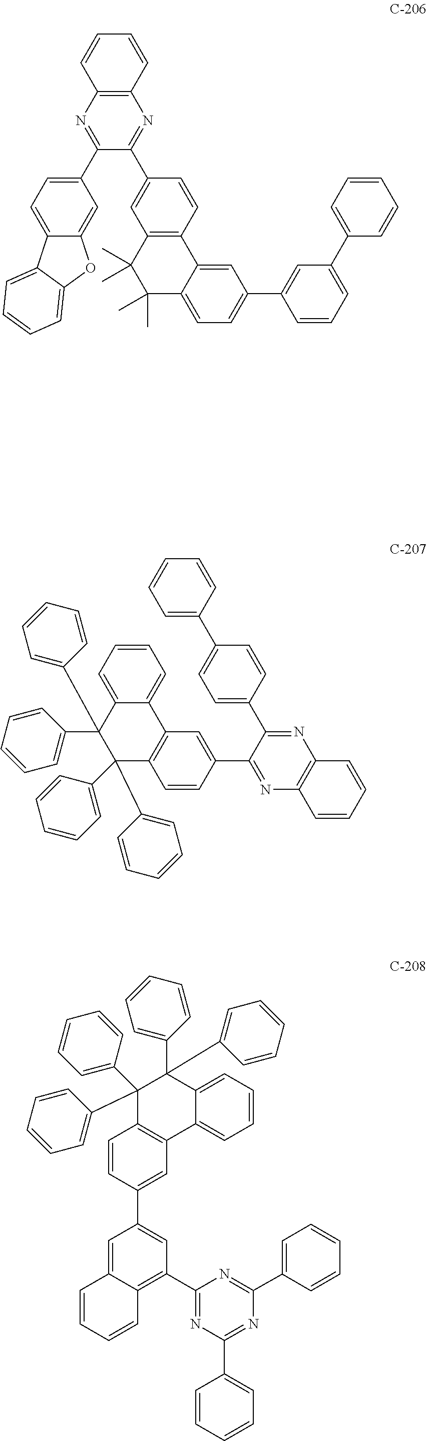 [SMILES: CC1(C)c2ccc(-c3cccc(-c4ccccc4)c3)cc2-c2ccc(-c3nc4ccccc4nc3-c3ccc4c(c3)oc3ccccc34)cc2C1(C)C ; c1ccc(-c2ccc(-c3nc4ccccc4nc3-c3ccc4c(c3)-c3ccccc3C(c3ccccc3)(c3ccccc3)C4(c3ccccc3)c3ccccc3)cc2)cc1 ; c1ccc(-c2nc(-c3ccccc3)nc(-c3cc(-c4ccc5c(c4)-c4ccccc4C(c4ccccc4)(c4ccccc4)C5(c4ccccc4)c4ccccc4)cc4ccccc34)n2)cc1]

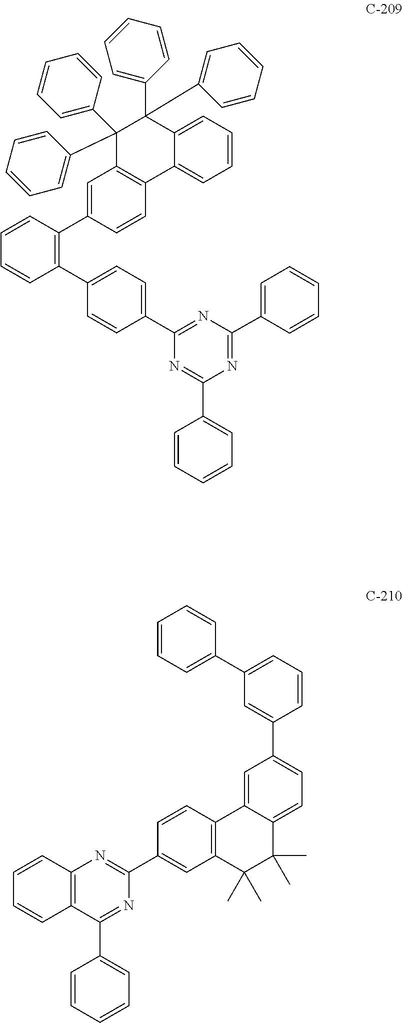 [SMILES: c1ccc(-c2nc(-c3ccccc3)nc(-c3ccc(-c4ccccc4-c4ccc5c(c4)C(c4ccccc4)(c4ccccc4)C(c4ccccc4)(c4ccccc4)c4ccccc4-5)cc3)n2)cc1 ; CC1(C)c2ccc(-c3cccc(-c4ccccc4)c3)cc2-c2ccc(-c3nc(-c4ccccc4)c4ccccc4n3)cc2C1(C)C]

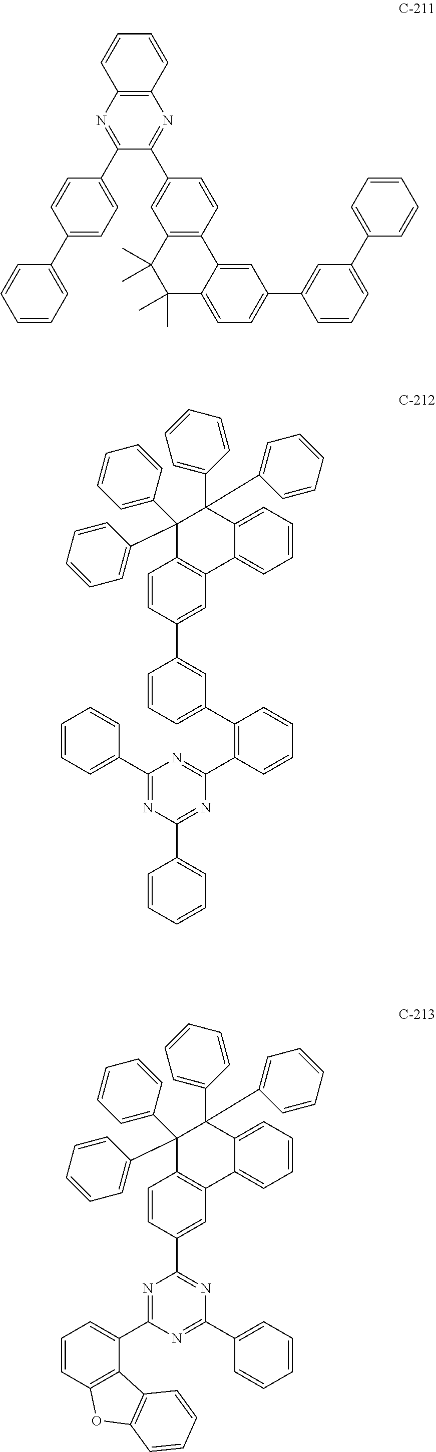 [SMILES: CC1(C)c2ccc(-c3cccc(-c4ccccc4)c3)cc2-c2ccc(-c3nc4ccccc4nc3-c3ccc(-c4ccccc4)cc3)cc2C1(C)C ; c1ccc(-c2nc(-c3ccccc3)nc(-c3ccccc3-c3cccc(-c4ccc5c(c4)-c4ccccc4C(c4ccccc4)(c4ccccc4)C5(c4ccccc4)c4ccccc4)c3)n2)cc1 ; c1ccc(-c2nc(-c3ccc4c(c3)-c3ccccc3C(c3ccccc3)(c3ccccc3)C4(c3ccccc3)c3ccccc3)nc(-c3cccc4oc5ccccc5c34)n2)cc1]

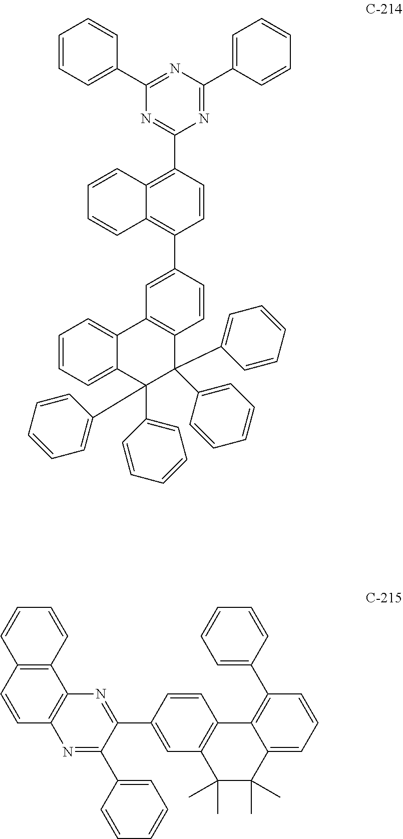 [SMILES: c1ccc(-c2nc(-c3ccccc3)nc(-c3ccc(-c4ccc5c(c4)-c4ccccc4C(c4ccccc4)(c4ccccc4)C5(c4ccccc4)c4ccccc4)c4ccccc34)n2)cc1 ; CC1(C)c2cc(-c3nc4c(ccc5ccccc54)nc3-c3ccccc3)ccc2-c2c(-c3ccccc3)cccc2C1(C)C]

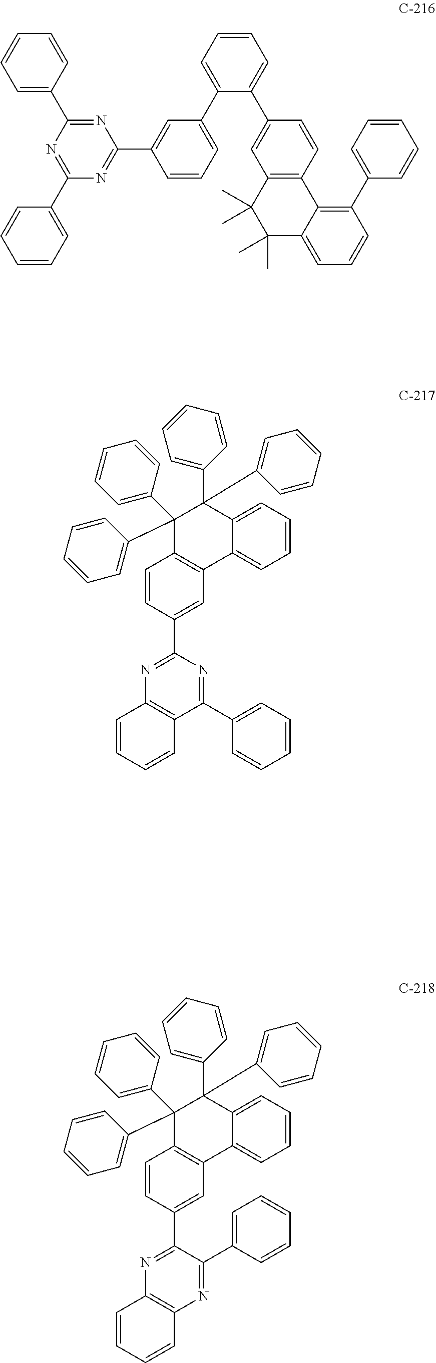 [SMILES: CC1(C)c2cc(-c3ccccc3-c3cccc(-c4nc(-c5ccccc5)nc(-c5ccccc5)n4)c3)ccc2-c2c(-c3ccccc3)cccc2C1(C)C ; c1ccc(-c2nc(-c3ccc4c(c3)-c3ccccc3C(c3ccccc3)(c3ccccc3)C4(c3ccccc3)c3ccccc3)nc3ccccc23)cc1 ; c1ccc(-c2nc3ccccc3nc2-c2ccc3c(c2)-c2ccccc2C(c2ccccc2)(c2ccccc2)C3(c2ccccc2)c2ccccc2)cc1]

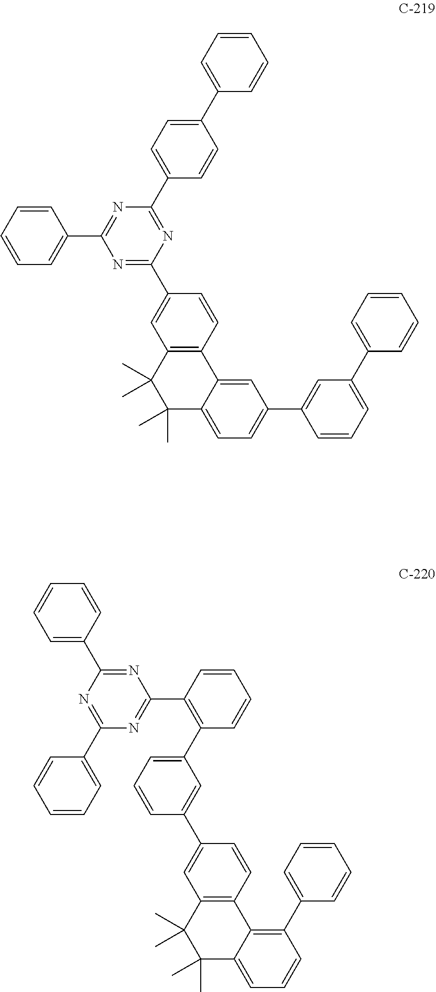 [SMILES: CC1(C)c2ccc(-c3cccc(-c4ccccc4)c3)cc2-c2ccc(-c3nc(-c4ccccc4)nc(-c4ccc(-c5ccccc5)cc4)n3)cc2C1(C)C ; CC1(C)c2cc(-c3cccc(-c4ccccc4-c4nc(-c5ccccc5)nc(-c5ccccc5)n4)c3)ccc2-c2c(-c3ccccc3)cccc2C1(C)C]

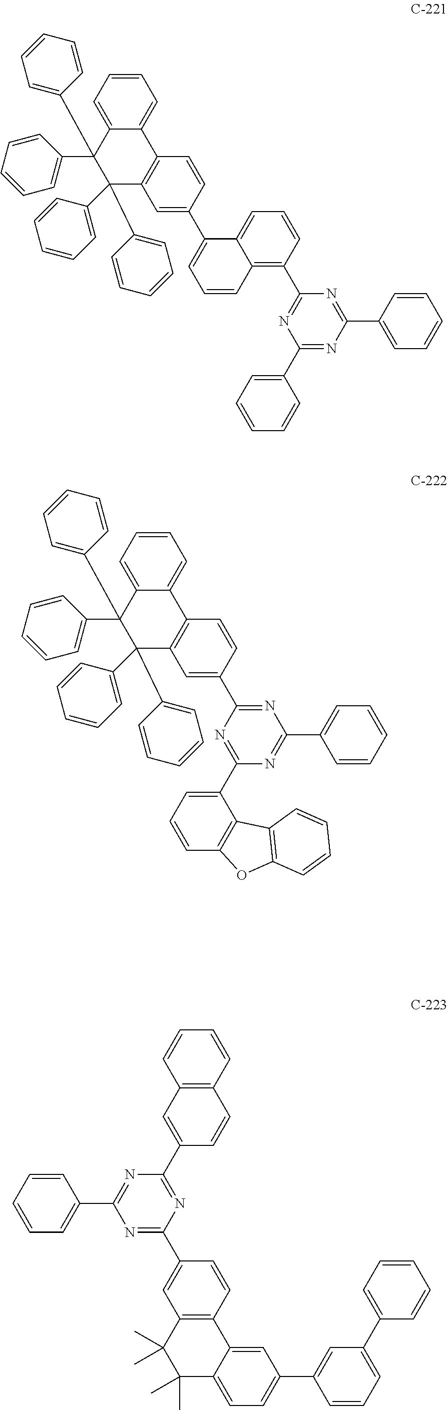 [SMILES: c1ccc(-c2nc(-c3ccccc3)nc(-c3cccc4c(-c5ccc6c(c5)C(c5ccccc5)(c5ccccc5)C(c5ccccc5)(c5ccccc5)c5ccccc5-6)cccc34)n2)cc1 ; c1ccc(-c2nc(-c3ccc4c(c3)C(c3ccccc3)(c3ccccc3)C(c3ccccc3)(c3ccccc3)c3ccccc3-4)nc(-c3cccc4oc5ccccc5c34)n2)cc1 ; CC1(C)c2ccc(-c3cccc(-c4ccccc4)c3)cc2-c2ccc(-c3nc(-c4ccccc4)nc(-c4ccc5ccccc5c4)n3)cc2C1(C)C]

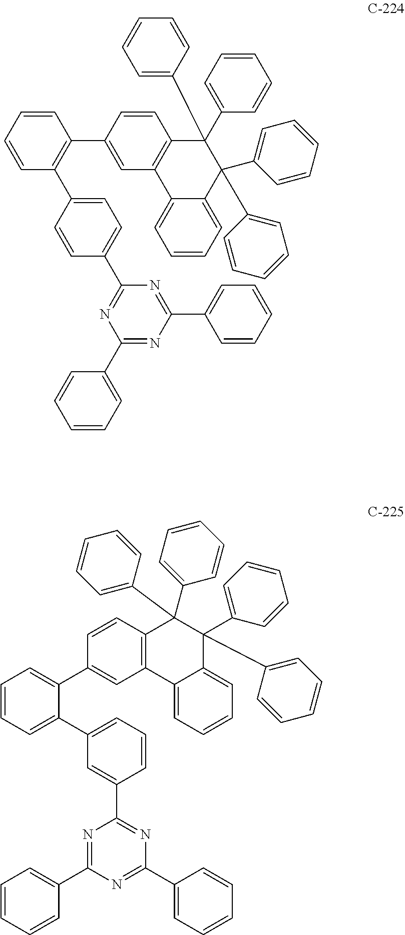 [SMILES: c1ccc(-c2nc(-c3ccccc3)nc(-c3ccc(-c4ccccc4-c4ccc5c(c4)-c4ccccc4C(c4ccccc4)(c4ccccc4)C5(c4ccccc4)c4ccccc4)cc3)n2)cc1 ; c1ccc(-c2nc(-c3ccccc3)nc(-c3cccc(-c4ccccc4-c4ccc5c(c4)-c4ccccc4C(c4ccccc4)(c4ccccc4)C5(c4ccccc4)c4ccccc4)c3)n2)cc1]

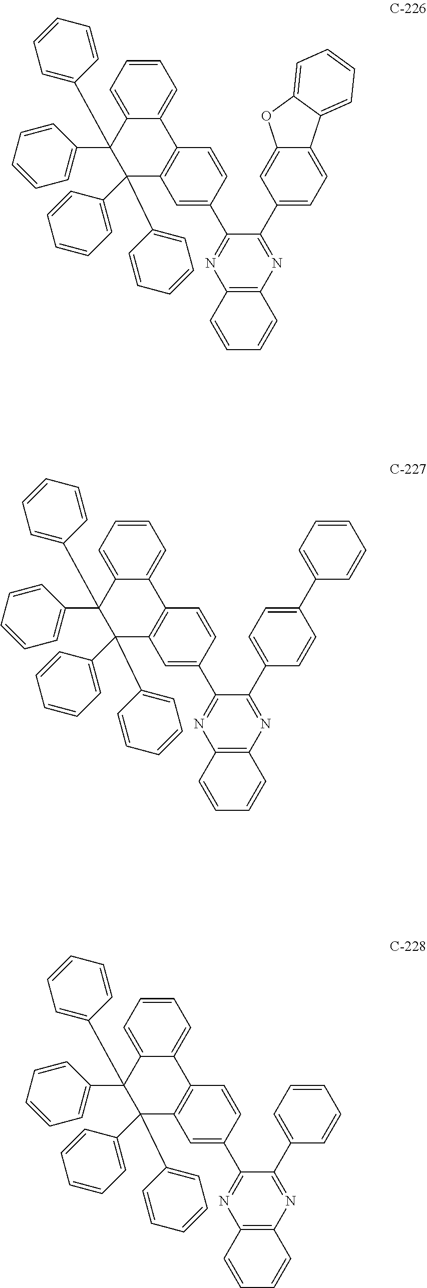 [SMILES: c1ccc(C2(c3ccccc3)c3ccccc3-c3ccc(-c4nc5ccccc5nc4-c4ccc5c(c4)oc4ccccc45)cc3C2(c2ccccc2)c2ccccc2)cc1 ; c1ccc(-c2ccc(-c3nc4ccccc4nc3-c3ccc4c(c3)C(c3ccccc3)(c3ccccc3)C(c3ccccc3)(c3ccccc3)c3ccccc3-4)cc2)cc1 ; c1ccc(-c2nc3ccccc3nc2-c2ccc3c(c2)C(c2ccccc2)(c2ccccc2)C(c2ccccc2)(c2ccccc2)c2ccccc2-3)cc1]

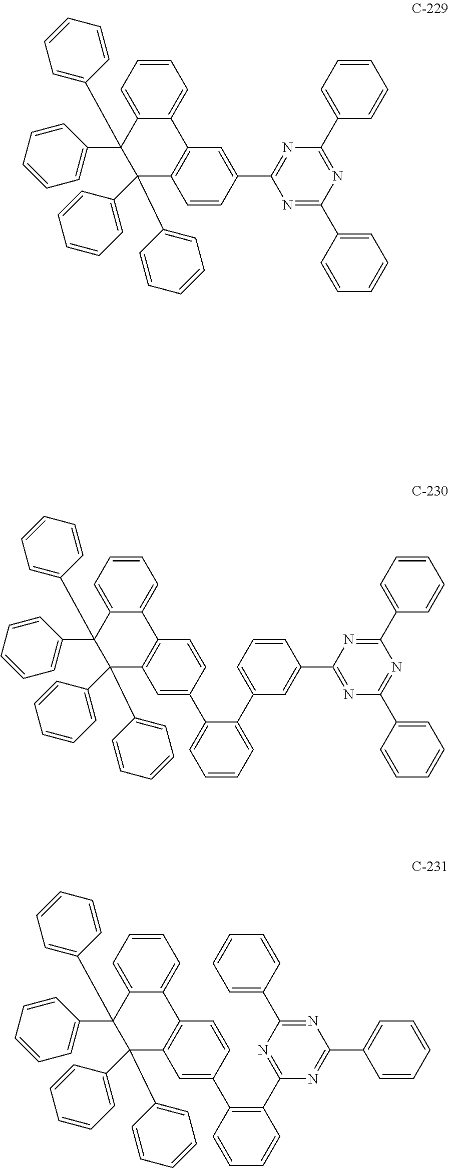 [SMILES: c1ccc(-c2nc(-c3ccccc3)nc(-c3ccc4c(c3)-c3ccccc3C(c3ccccc3)(c3ccccc3)C4(c3ccccc3)c3ccccc3)n2)cc1 ; c1ccc(-c2nc(-c3ccccc3)nc(-c3cccc(-c4ccccc4-c4ccc5c(c4)C(c4ccccc4)(c4ccccc4)C(c4ccccc4)(c4ccccc4)c4ccccc4-5)c3)n2)cc1 ; c1ccc(-c2nc(-c3ccccc3)nc(-c3ccccc3-c3ccc4c(c3)C(c3ccccc3)(c3ccccc3)C(c3ccccc3)(c3ccccc3)c3ccccc3-4)n2)cc1]

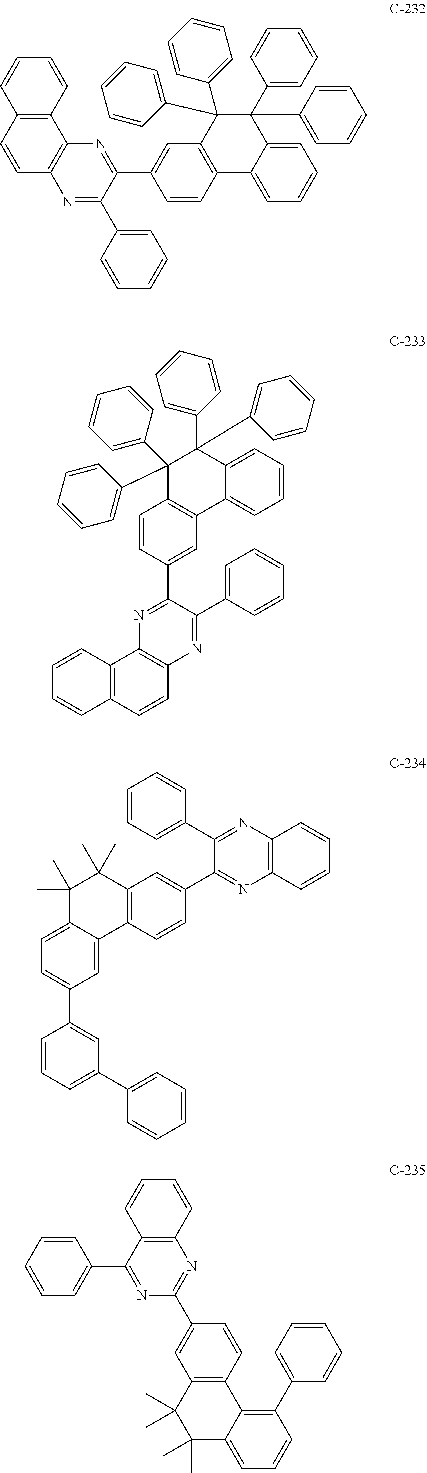

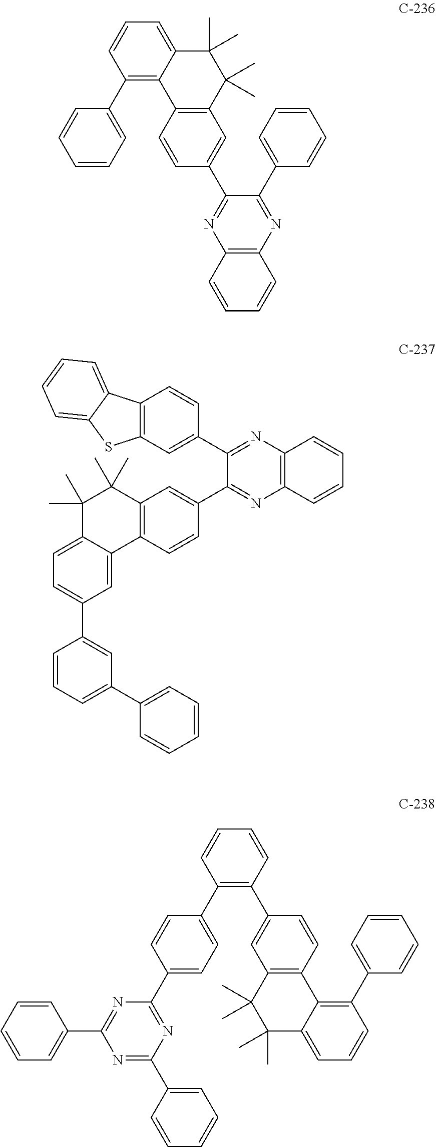 [SMILES: CC1(C)c2cc(-c3nc4ccccc4nc3-c3ccccc3)ccc2-c2c(-c3ccccc3)cccc2C1(C)C ; CC1(C)c2ccc(-c3cccc(-c4ccccc4)c3)cc2-c2ccc(-c3nc4ccccc4nc3-c3ccc4c(c3)sc3ccccc34)cc2C1(C)C ; CC1(C)c2cc(-c3ccccc3-c3ccc(-c4nc(-c5ccccc5)nc(-c5ccccc5)n4)cc3)ccc2-c2c(-c3ccccc3)cccc2C1(C)C]

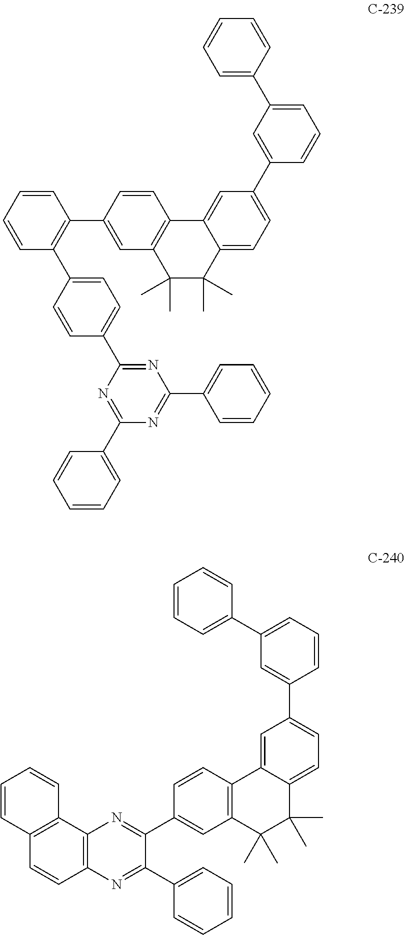 [SMILES: CC1(C)c2ccc(-c3cccc(-c4ccccc4)c3)cc2-c2ccc(-c3ccccc3-c3ccc(-c4nc(-c5ccccc5)nc(-c5ccccc5)n4)cc3)cc2C1(C)C ; CC1(C)c2ccc(-c3cccc(-c4ccccc4)c3)cc2-c2ccc(-c3nc4c(ccc5ccccc54)nc3-c3ccccc3)cc2C1(C)C]

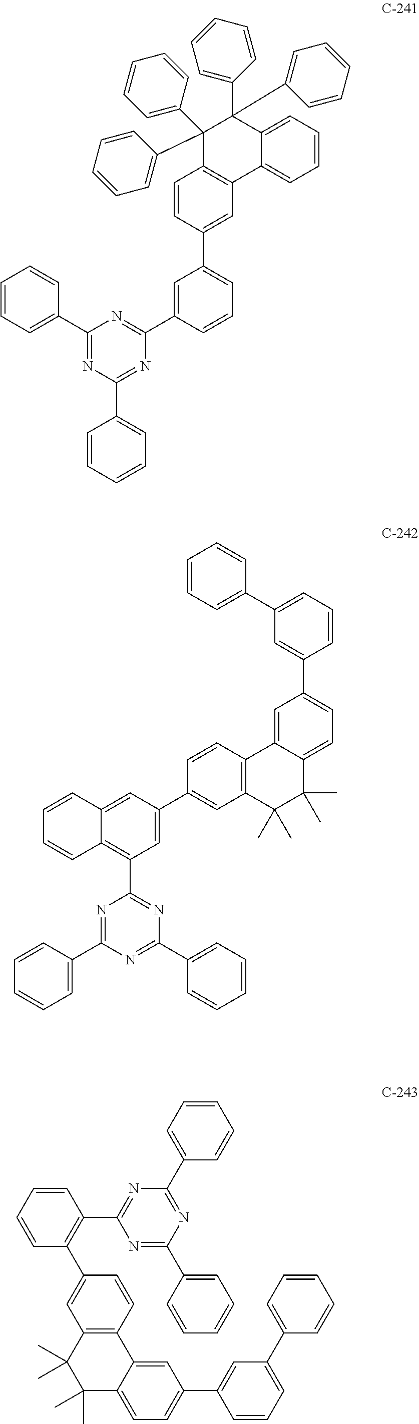 [SMILES: c1ccc(-c2nc(-c3ccccc3)nc(-c3cccc(-c4ccc5c(c4)-c4ccccc4C(c4ccccc4)(c4ccccc4)C5(c4ccccc4)c4ccccc4)c3)n2)cc1 ; CC1(C)c2ccc(-c3cccc(-c4ccccc4)c3)cc2-c2ccc(-c3cc(-c4nc(-c5ccccc5)nc(-c5ccccc5)n4)c4ccccc4c3)cc2C1(C)C ; CC1(C)c2ccc(-c3cccc(-c4ccccc4)c3)cc2-c2ccc(-c3ccccc3-c3nc(-c4ccccc4)nc(-c4ccccc4)n3)cc2C1(C)C]

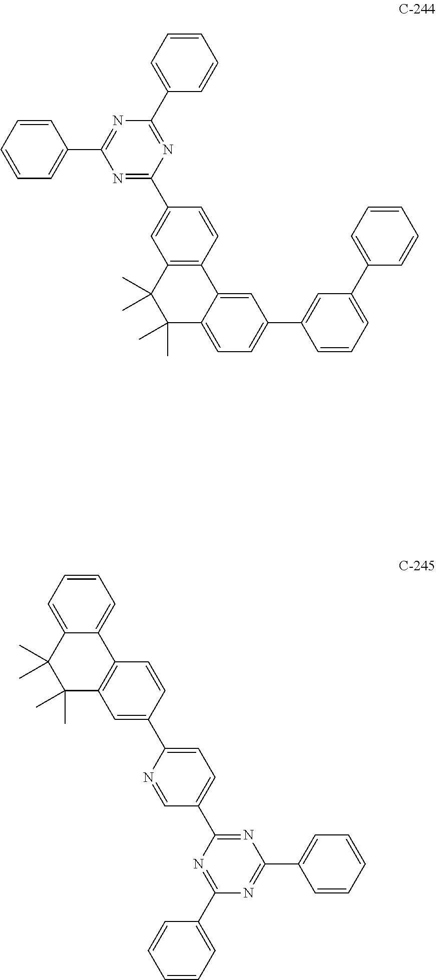 [SMILES: CC1(C)c2ccc(-c3cccc(-c4ccccc4)c3)cc2-c2ccc(-c3nc(-c4ccccc4)nc(-c4ccccc4)n3)cc2C1(C)C ; CC1(C)c2ccccc2-c2ccc(-c3ccc(-c4nc(-c5ccccc5)nc(-c5ccccc5)n4)cn3)cc2C1(C)C]

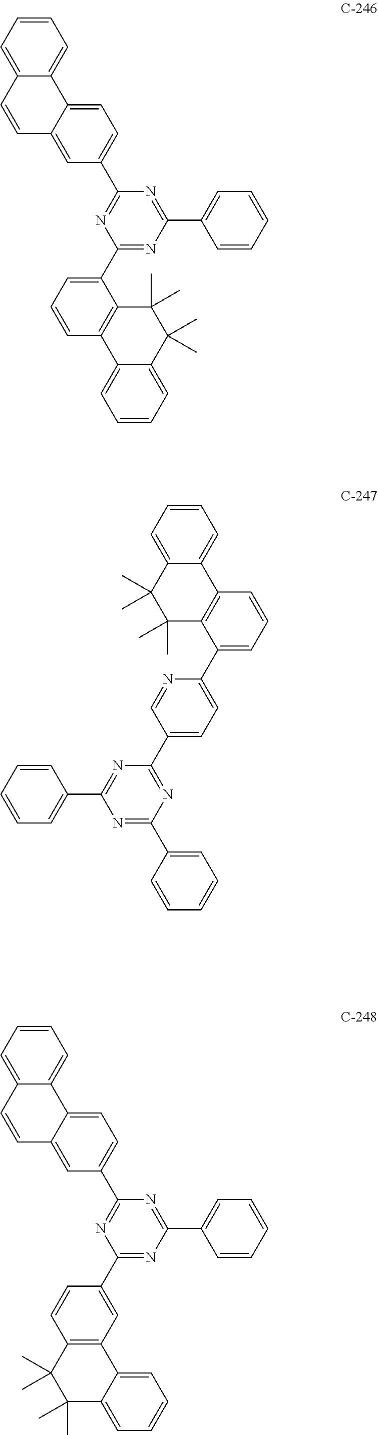 [SMILES: CC1(C)c2ccccc2-c2cccc(-c3nc(-c4ccccc4)nc(-c4ccc5c(ccc6ccccc65)c4)n3)c2C1(C)C ; CC1(C)c2ccccc2-c2cccc(-c3ccc(-c4nc(-c5ccccc5)nc(-c5ccccc5)n4)cn3)c2C1(C)C ; CC1(C)c2ccccc2-c2cc(-c3nc(-c4ccccc4)nc(-c4ccc5c(ccc6ccccc65)c4)n3)ccc2C1(C)C]

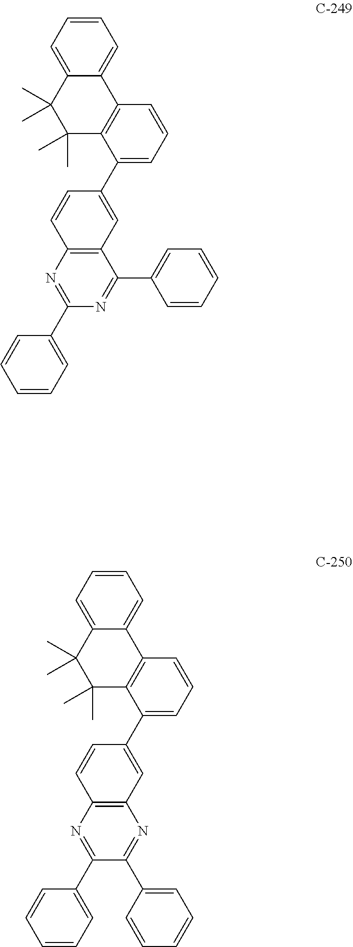 [SMILES: CC1(C)c2ccccc2-c2cccc(-c3ccc4nc(-c5ccccc5)nc(-c5ccccc5)c4c3)c2C1(C)C ; CC1(C)c2ccccc2-c2cccc(-c3ccc4nc(-c5ccccc5)c(-c5ccccc5)nc4c3)c2C1(C)C]

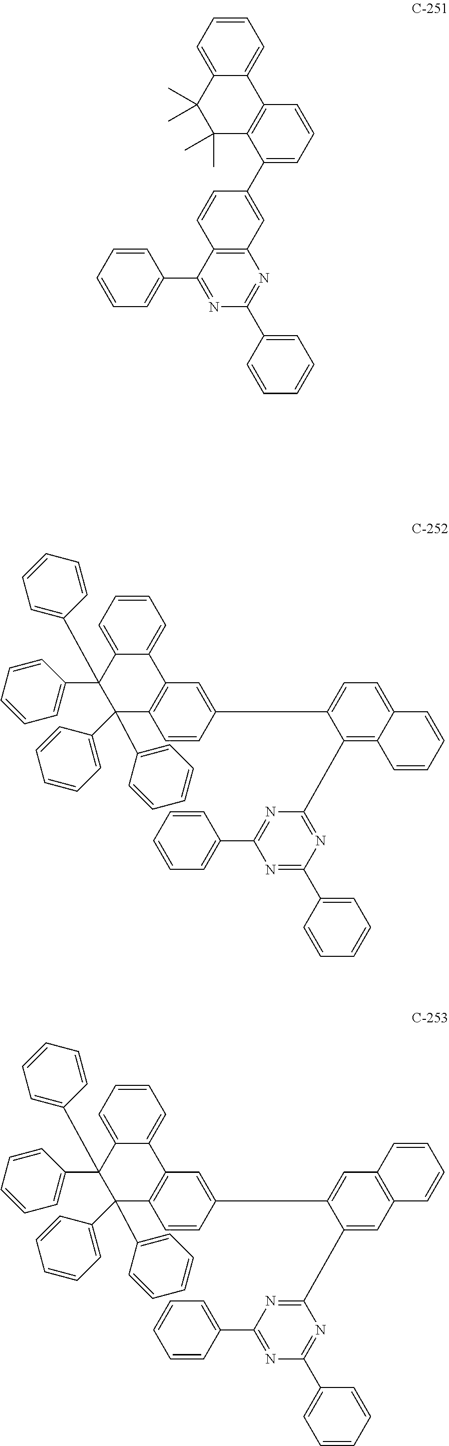 [SMILES: CC1(C)c2ccccc2-c2cccc(-c3ccc4c(-c5ccccc5)nc(-c5ccccc5)nc4c3)c2C1(C)C ; c1ccc(-c2nc(-c3ccccc3)nc(-c3c(-c4ccc5c(c4)-c4ccccc4C(c4ccccc4)(c4ccccc4)C5(c4ccccc4)c4ccccc4)ccc4ccccc34)n2)cc1 ; c1ccc(-c2nc(-c3ccccc3)nc(-c3cc4ccccc4cc3-c3ccc4c(c3)-c3ccccc3C(c3ccccc3)(c3ccccc3)C4(c3ccccc3)c3ccccc3)n2)cc1]

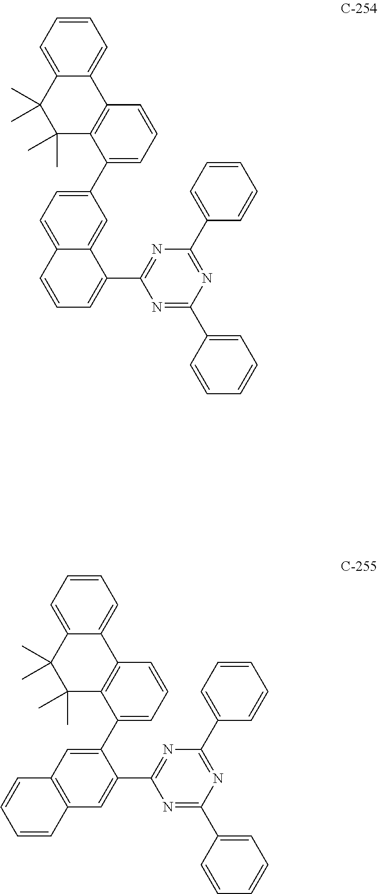 [SMILES: CC1(C)c2ccccc2-c2cccc(-c3ccc4cccc(-c5nc(-c6ccccc6)nc(-c6ccccc6)n5)c4c3)c2C1(C)C ; CC1(C)c2ccccc2-c2cccc(-c3cc4ccccc4cc3-c3nc(-c4ccccc4)nc(-c4ccccc4)n3)c2C1(C)C]

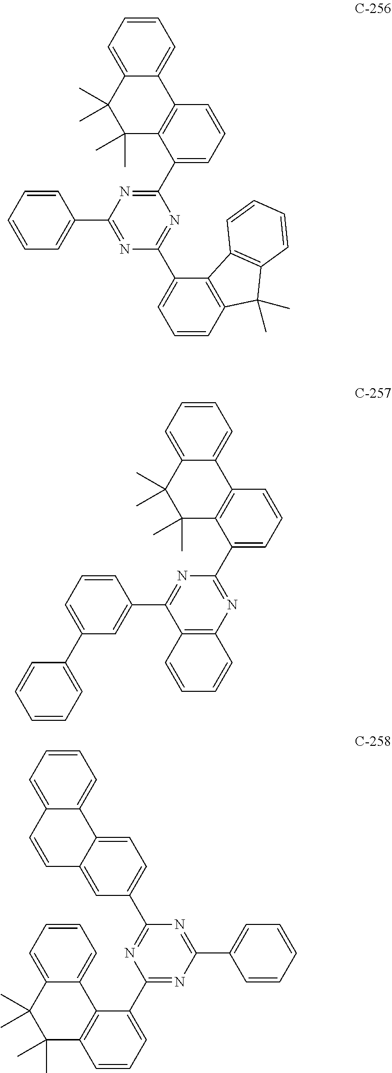

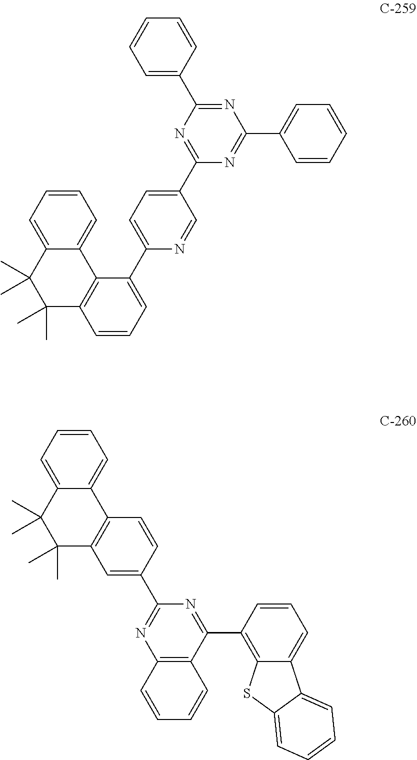 [SMILES: CC1(C)c2ccccc2-c2c(-c3ccc(-c4nc(-c5ccccc5)nc(-c5ccccc5)n4)cn3)cccc2C1(C)C ; CC1(C)c2ccccc2-c2ccc(-c3nc(-c4cccc5c4sc4ccccc45)c4ccccc4n3)cc2C1(C)C]

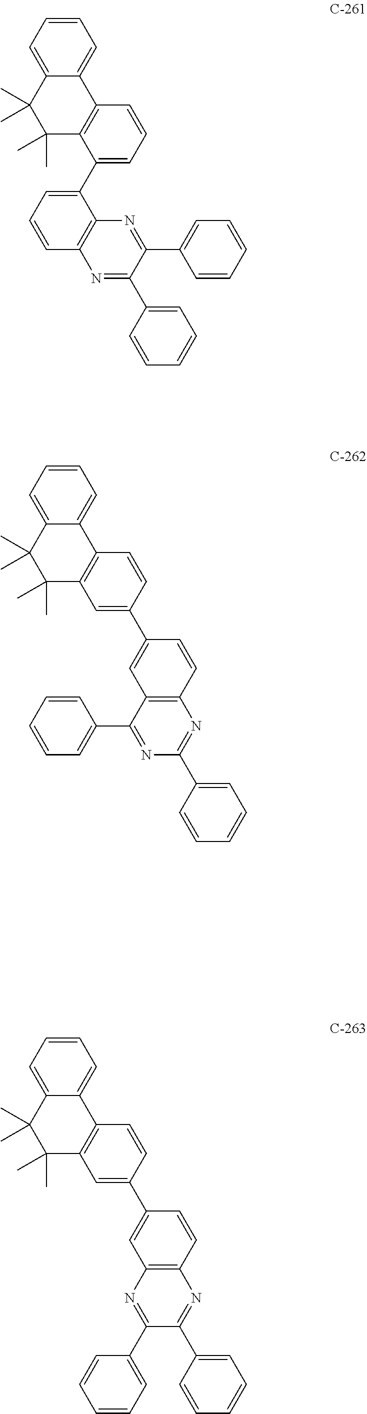 [SMILES: CC1(C)c2ccccc2-c2cccc(-c3cccc4nc(-c5ccccc5)c(-c5ccccc5)nc34)c2C1(C)C ; CC1(C)c2ccccc2-c2ccc(-c3ccc4nc(-c5ccccc5)nc(-c5ccccc5)c4c3)cc2C1(C)C ; CC1(C)c2ccccc2-c2ccc(-c3ccc4nc(-c5ccccc5)c(-c5ccccc5)nc4c3)cc2C1(C)C]

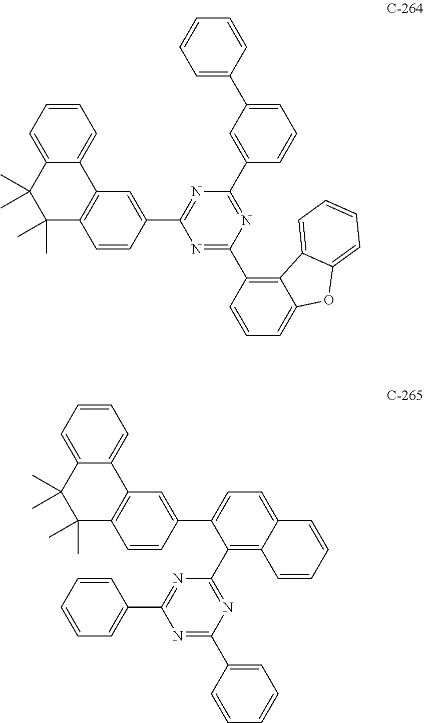 [SMILES: CC1(C)c2ccccc2-c2cc(-c3nc(-c4cccc(-c5ccccc5)c4)nc(-c4cccc5oc6ccccc6c45)n3)ccc2C1(C)C ; CC1(C)c2ccccc2-c2cc(-c3ccc4ccccc4c3-c3nc(-c4ccccc4)nc(-c4ccccc4)n3)ccc2C1(C)C]

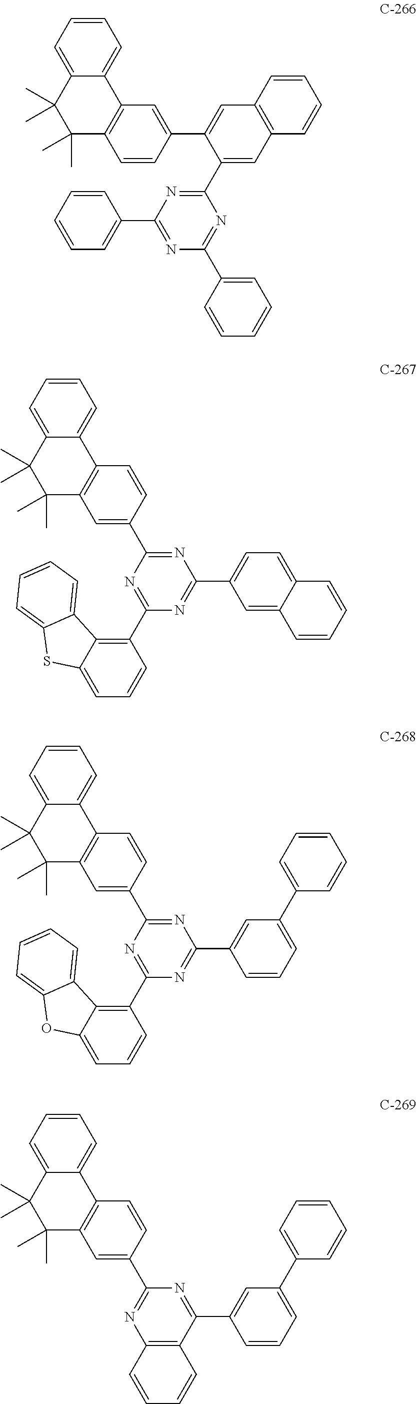 [SMILES: CC1(C)c2ccccc2-c2cc(-c3cc4ccccc4cc3-c3nc(-c4ccccc4)nc(-c4ccccc4)n3)ccc2C1(C)C ; CC1(C)c2ccccc2-c2ccc(-c3nc(-c4ccc5ccccc5c4)nc(-c4cccc5sc6ccccc6c45)n3)cc2C1(C)C ; CC1(C)c2ccccc2-c2ccc(-c3nc(-c4cccc(-c5ccccc5)c4)nc(-c4cccc5oc6ccccc6c45)n3)cc2C1(C)C ; CC1(C)c2ccccc2-c2ccc(-c3nc(-c4cccc(-c5ccccc5)c4)c4ccccc4n3)cc2C1(C)C]

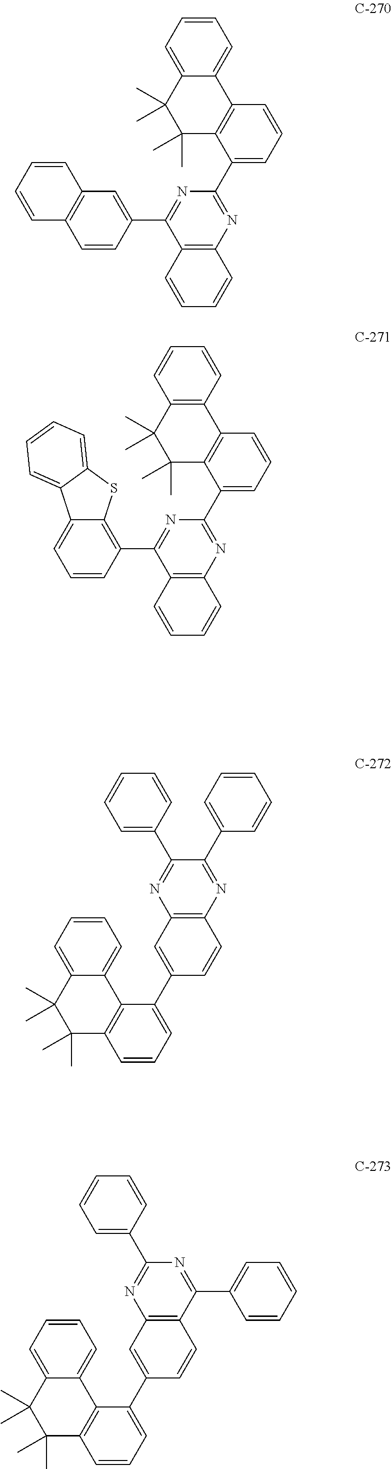

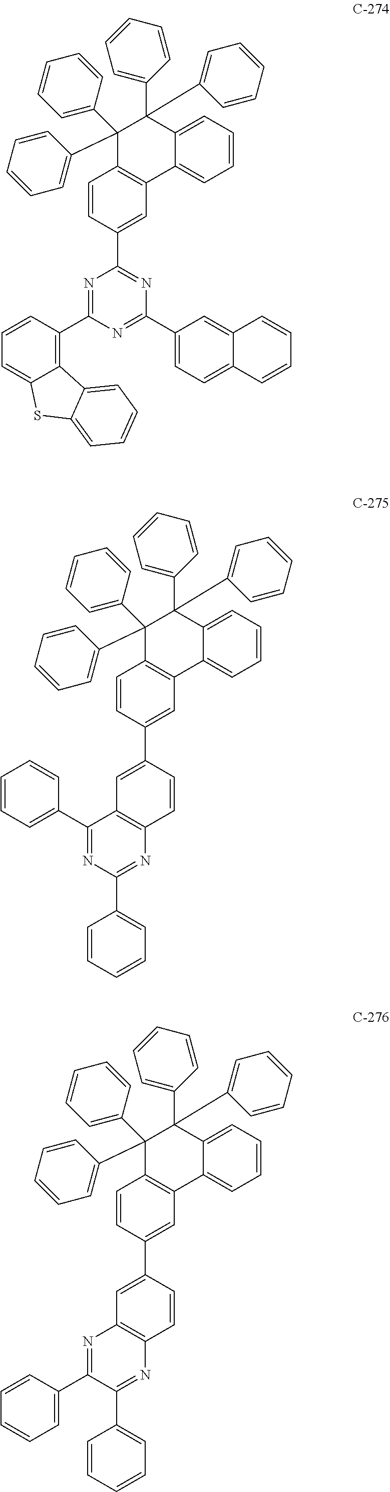 [SMILES: c1ccc(C2(c3ccccc3)c3ccccc3-c3cc(-c4nc(-c5ccc6ccccc6c5)nc(-c5cccc6sc7ccccc7c56)n4)ccc3C2(c2ccccc2)c2ccccc2)cc1 ; c1ccc(-c2nc(-c3ccccc3)c3cc(-c4ccc5c(c4)-c4ccccc4C(c4ccccc4)(c4ccccc4)C5(c4ccccc4)c4ccccc4)ccc3n2)cc1 ; c1ccc(-c2nc3ccc(-c4ccc5c(c4)-c4ccccc4C(c4ccccc4)(c4ccccc4)C5(c4ccccc4)c4ccccc4)cc3nc2-c2ccccc2)cc1]

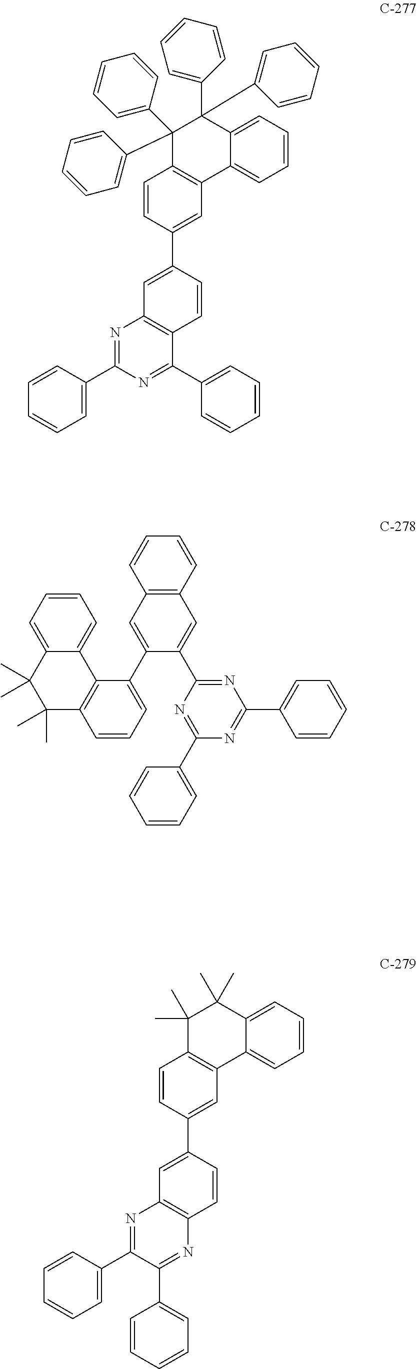 [SMILES: c1ccc(-c2nc(-c3ccccc3)c3ccc(-c4ccc5c(c4)-c4ccccc4C(c4ccccc4)(c4ccccc4)C5(c4ccccc4)c4ccccc4)cc3n2)cc1 ; CC1(C)c2ccccc2-c2c(-c3cc4ccccc4cc3-c3nc(-c4ccccc4)nc(-c4ccccc4)n3)cccc2C1(C)C ; CC1(C)c2ccccc2-c2cc(-c3ccc4nc(-c5ccccc5)c(-c5ccccc5)nc4c3)ccc2C1(C)C]

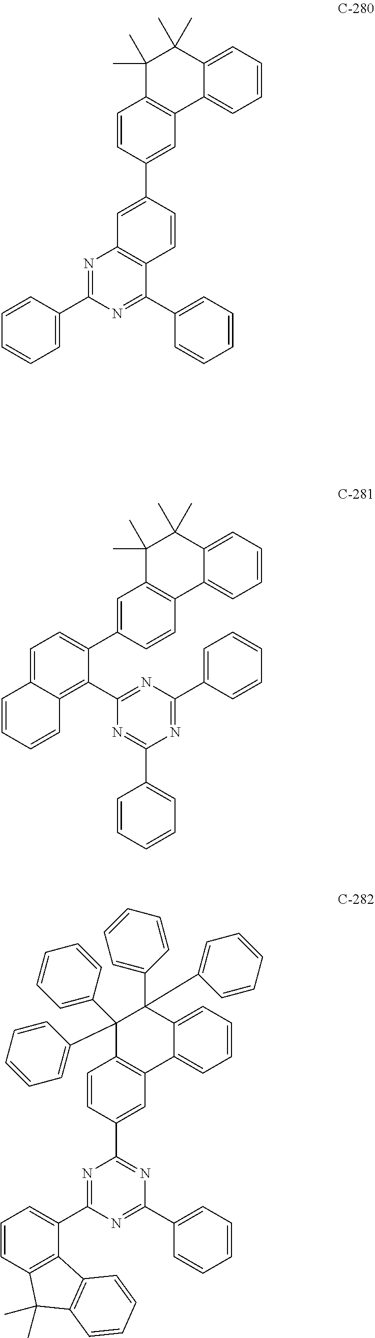 [SMILES: CC1(C)c2ccccc2-c2cc(-c3ccc4c(-c5ccccc5)nc(-c5ccccc5)nc4c3)ccc2C1(C)C ; CC1(C)c2ccccc2-c2ccc(-c3ccc4ccccc4c3-c3nc(-c4ccccc4)nc(-c4ccccc4)n3)cc2C1(C)C ; CC1(C)c2ccccc2-c2c(-c3nc(-c4ccccc4)nc(-c4ccc5c(c4)-c4ccccc4C(c4ccccc4)(c4ccccc4)C5(c4ccccc4)c4ccccc4)n3)cccc21]

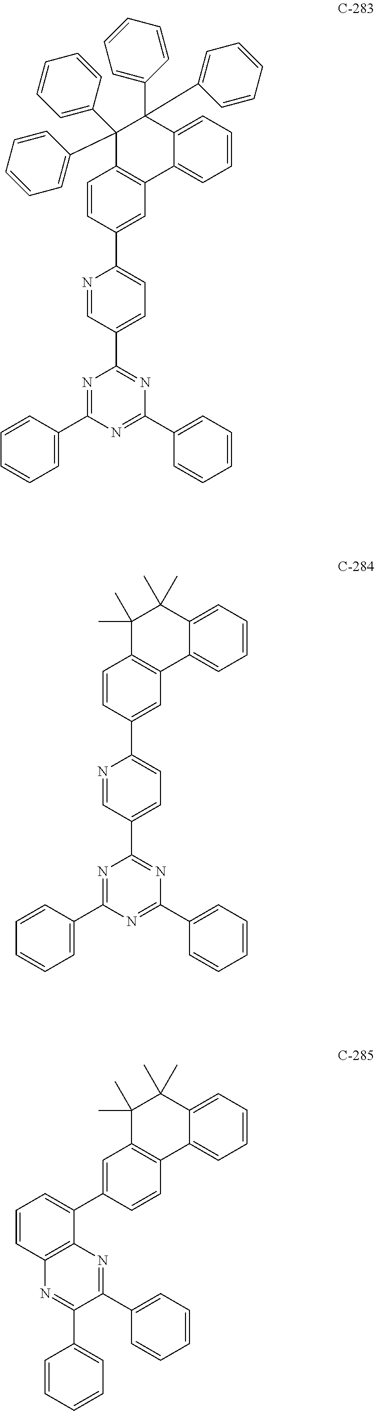 [SMILES: c1ccc(-c2nc(-c3ccccc3)nc(-c3ccc(-c4ccc5c(c4)-c4ccccc4C(c4ccccc4)(c4ccccc4)C5(c4ccccc4)c4ccccc4)nc3)n2)cc1 ; CC1(C)c2ccccc2-c2cc(-c3ccc(-c4nc(-c5ccccc5)nc(-c5ccccc5)n4)cn3)ccc2C1(C)C ; CC1(C)c2ccccc2-c2ccc(-c3cccc4nc(-c5ccccc5)c(-c5ccccc5)nc34)cc2C1(C)C]

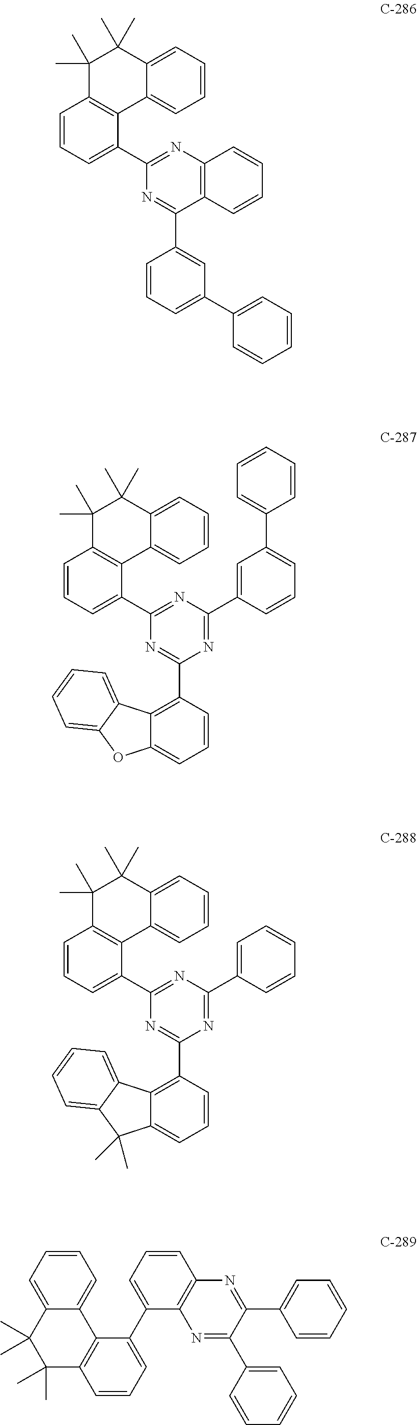 [SMILES: CC1(C)c2ccccc2-c2c(-c3nc(-c4cccc(-c5ccccc5)c4)c4ccccc4n3)cccc2C1(C)C ; CC1(C)c2ccccc2-c2c(-c3nc(-c4cccc(-c5ccccc5)c4)nc(-c4cccc5oc6ccccc6c45)n3)cccc2C1(C)C ; CC1(C)c2ccccc2-c2c(-c3nc(-c4ccccc4)nc(-c4cccc5c4-c4ccccc4C(C)(C)C5(C)C)n3)cccc21 ; CC1(C)c2ccccc2-c2c(-c3cccc4nc(-c5ccccc5)c(-c5ccccc5)nc34)cccc2C1(C)C]

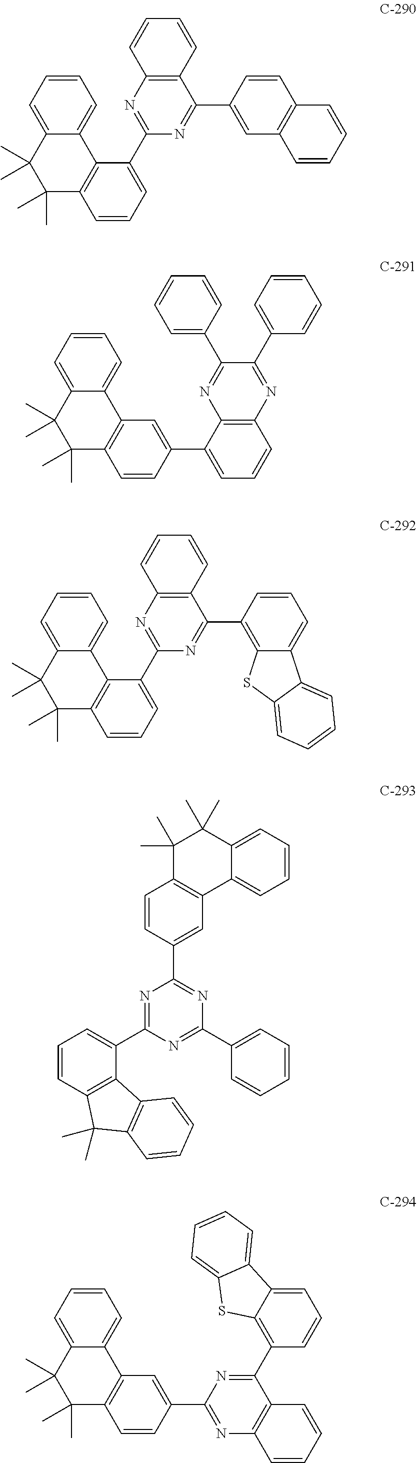 [SMILES: CC1(C)c2ccccc2-c2c(-c3nc(-c4ccc5ccccc5c4)c4ccccc4n3)cccc2C1(C)C ; CC1(C)c2ccccc2-c2cc(-c3cccc4nc(-c5ccccc5)c(-c5ccccc5)nc34)ccc2C1(C)C ; CC1(C)c2ccccc2-c2c(-c3nc(-c4cccc5c4sc4ccccc45)c4ccccc4n3)cccc2C1(C)C ; CC1(C)c2ccccc2-c2c(-c3nc(-c4ccccc4)nc(-c4ccc5c(c4)-c4ccccc4C(C)(C)C5(C)C)n3)cccc21 ; CC1(C)c2ccccc2-c2cc(-c3nc(-c4cccc5c4sc4ccccc45)c4ccccc4n3)ccc2C1(C)C]

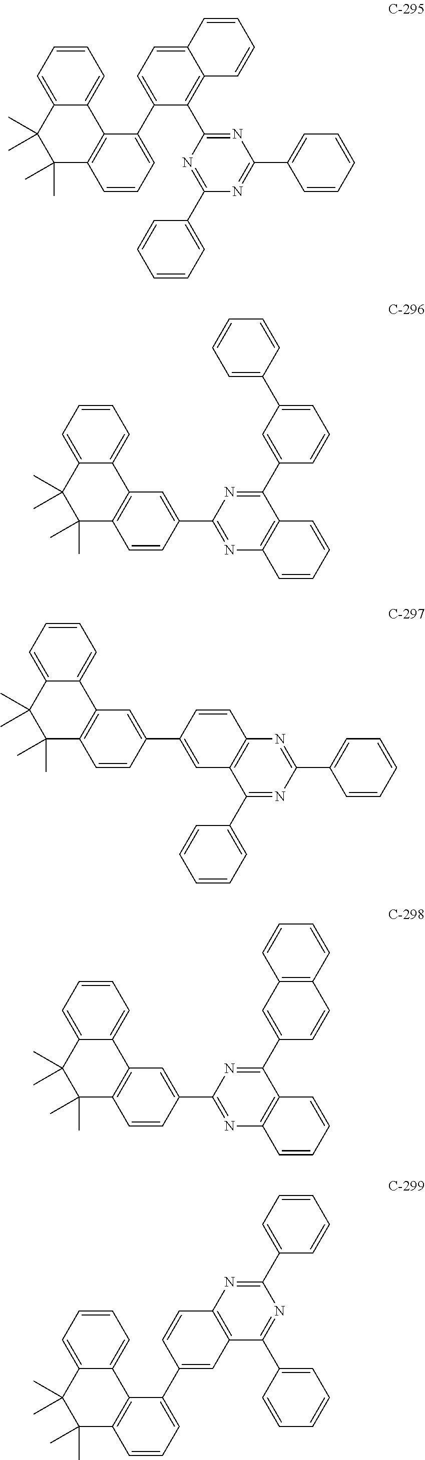

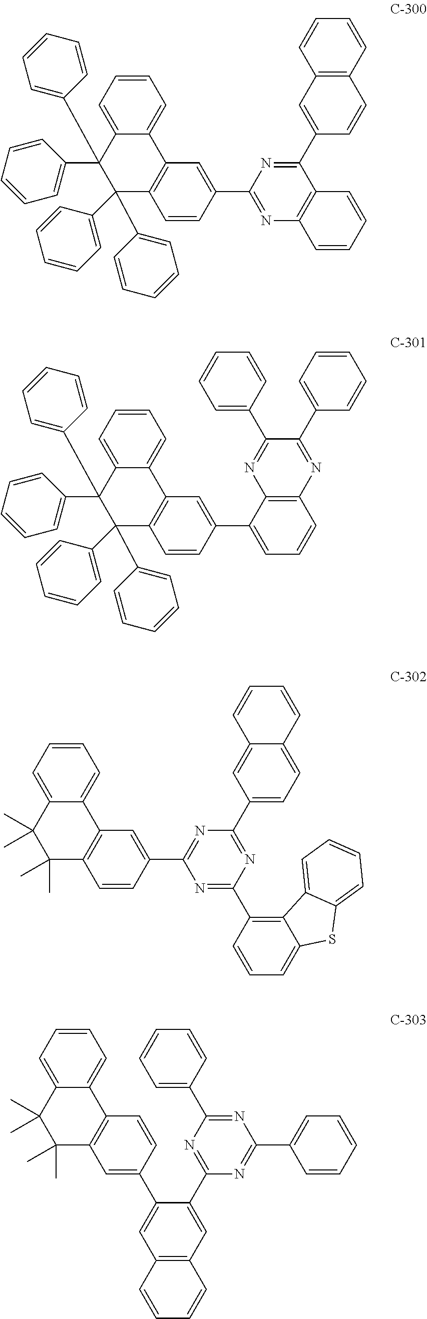 [SMILES: c1ccc(C2(c3ccccc3)c3ccccc3-c3cc(-c4nc(-c5ccc6ccccc6c5)c5ccccc5n4)ccc3C2(c2ccccc2)c2ccccc2)cc1 ; c1ccc(-c2nc3cccc(-c4ccc5c(c4)-c4ccccc4C(c4ccccc4)(c4ccccc4)C5(c4ccccc4)c4ccccc4)c3nc2-c2ccccc2)cc1 ; CC1(C)c2ccccc2-c2cc(-c3nc(-c4ccc5ccccc5c4)nc(-c4cccc5sc6ccccc6c45)n3)ccc2C1(C)C ; CC1(C)c2ccccc2-c2ccc(-c3cc4ccccc4cc3-c3nc(-c4ccccc4)nc(-c4ccccc4)n3)cc2C1(C)C]

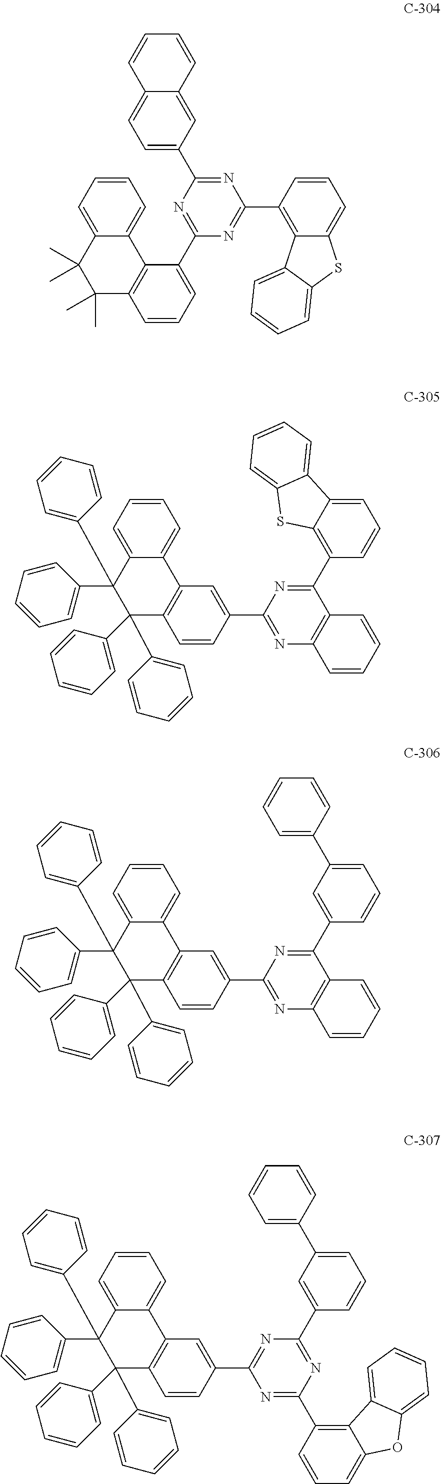 [SMILES: CC1(C)c2ccccc2-c2c(-c3nc(-c4ccc5ccccc5c4)nc(-c4cccc5sc6ccccc6c45)n3)cccc2C1(C)C ; c1ccc(C2(c3ccccc3)c3ccccc3-c3cc(-c4nc(-c5cccc6c5sc5ccccc56)c5ccccc5n4)ccc3C2(c2ccccc2)c2ccccc2)cc1 ; c1ccc(-c2cccc(-c3nc(-c4ccc5c(c4)-c4ccccc4C(c4ccccc4)(c4ccccc4)C5(c4ccccc4)c4ccccc4)nc4ccccc34)c2)cc1 ; c1ccc(-c2cccc(-c3nc(-c4ccc5c(c4)-c4ccccc4C(c4ccccc4)(c4ccccc4)C5(c4ccccc4)c4ccccc4)nc(-c4cccc5oc6ccccc6c45)n3)c2)cc1]

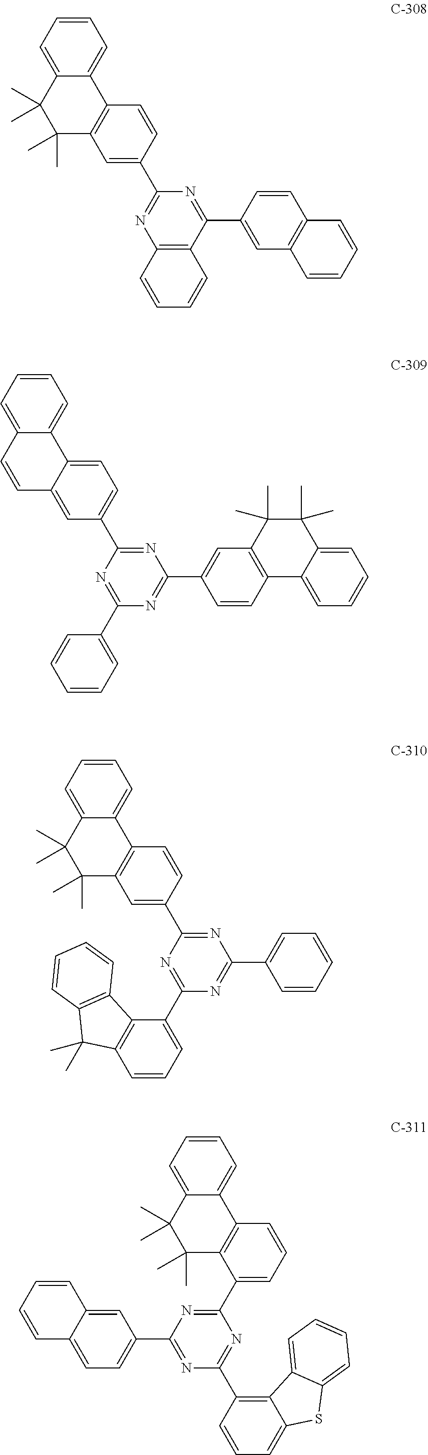 [SMILES: CC1(C)c2ccccc2-c2ccc(-c3nc(-c4ccc5ccccc5c4)c4ccccc4n3)cc2C1(C)C ; CC1(C)c2ccccc2-c2ccc(-c3nc(-c4ccccc4)nc(-c4ccc5c(ccc6ccccc65)c4)n3)cc2C1(C)C ; CC1(C)c2ccccc2-c2c(-c3nc(-c4ccccc4)nc(-c4ccc5c(c4)C(C)(C)C(C)(C)c4ccccc4-5)n3)cccc21 ; CC1(C)c2ccccc2-c2cccc(-c3nc(-c4ccc5ccccc5c4)nc(-c4cccc5sc6ccccc6c45)n3)c2C1(C)C]

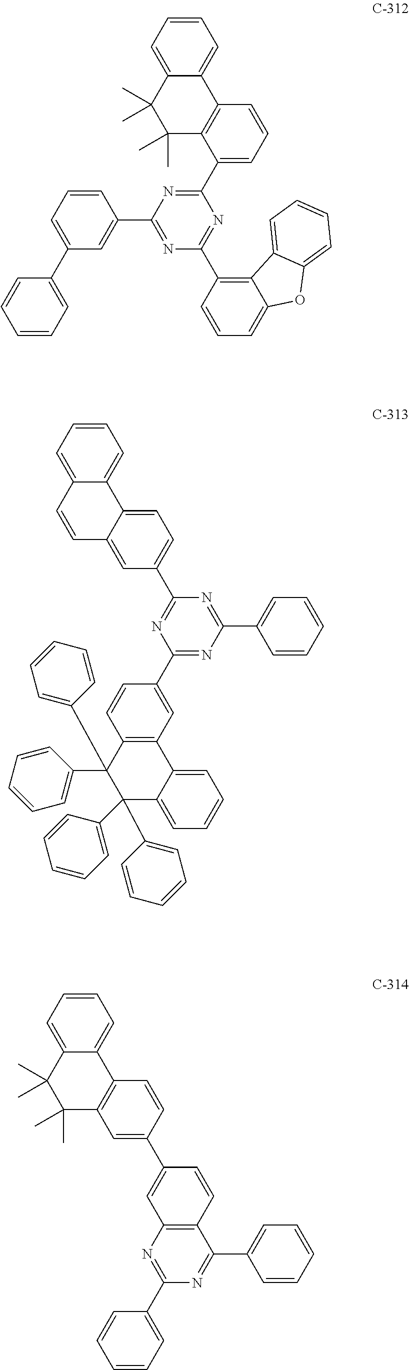 [SMILES: CC1(C)c2ccccc2-c2cccc(-c3nc(-c4cccc(-c5ccccc5)c4)nc(-c4cccc5oc6ccccc6c45)n3)c2C1(C)C ; c1ccc(-c2nc(-c3ccc4c(c3)-c3ccccc3C(c3ccccc3)(c3ccccc3)C4(c3ccccc3)c3ccccc3)nc(-c3ccc4c(ccc5ccccc54)c3)n2)cc1 ; CC1(C)c2ccccc2-c2ccc(-c3ccc4c(-c5ccccc5)nc(-c5ccccc5)nc4c3)cc2C1(C)C]

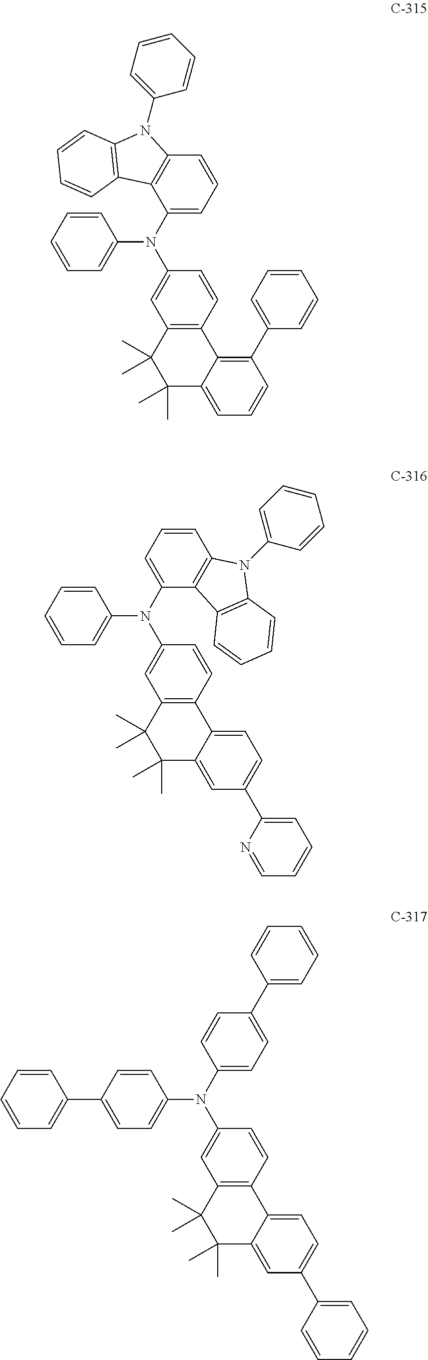

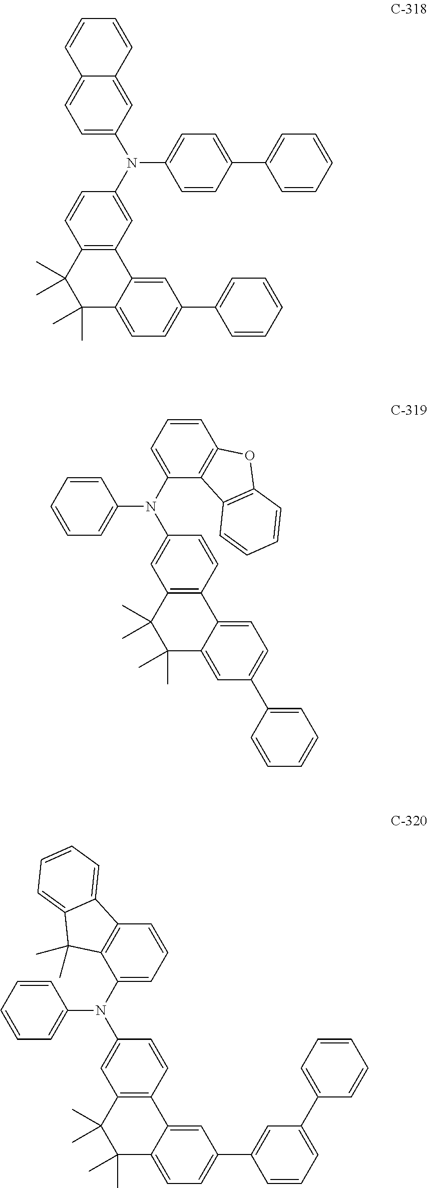 [SMILES: CC1(C)c2ccc(-c3ccccc3)cc2-c2cc(N(c3ccc(-c4ccccc4)cc3)c3ccc4ccccc4c3)ccc2C1(C)C ; CC1(C)c2cc(-c3ccccc3)ccc2-c2ccc(N(c3ccccc3)c3cccc4oc5ccccc5c34)cc2C1(C)C ; CC1(C)c2ccccc2-c2cccc(N(c3ccccc3)c3ccc4c(c3)C(C)(C)C(C)(C)c3ccc(-c5cccc(-c6ccccc6)c5)cc3-4)c21]

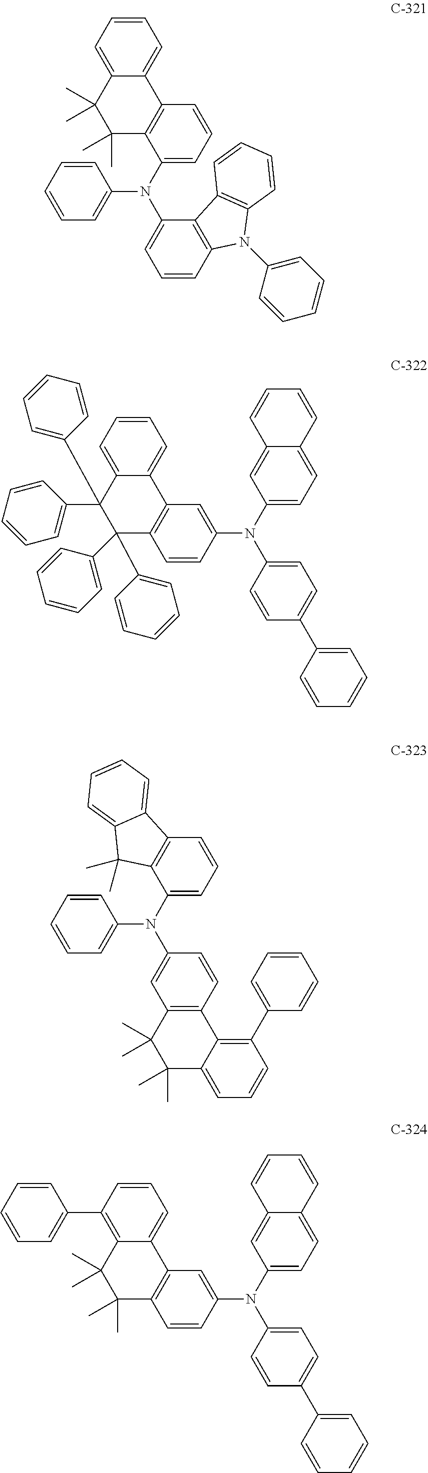 [SMILES: CC1(C)c2ccccc2-c2cccc(N(c3ccccc3)c3cccc4c3c3ccccc3n4-c3ccccc3)c2C1(C)C ; c1ccc(-c2ccc(N(c3ccc4c(c3)-c3ccccc3C(c3ccccc3)(c3ccccc3)C4(c3ccccc3)c3ccccc3)c3ccc4ccccc4c3)cc2)cc1 ; CC1(C)c2ccccc2-c2cccc(N(c3ccccc3)c3ccc4c(c3)C(C)(C)C(C)(C)c3cccc(-c5ccccc5)c3-4)c21 ; CC1(C)c2ccc(N(c3ccc(-c4ccccc4)cc3)c3ccc4ccccc4c3)cc2-c2cccc(-c3ccccc3)c2C1(C)C]

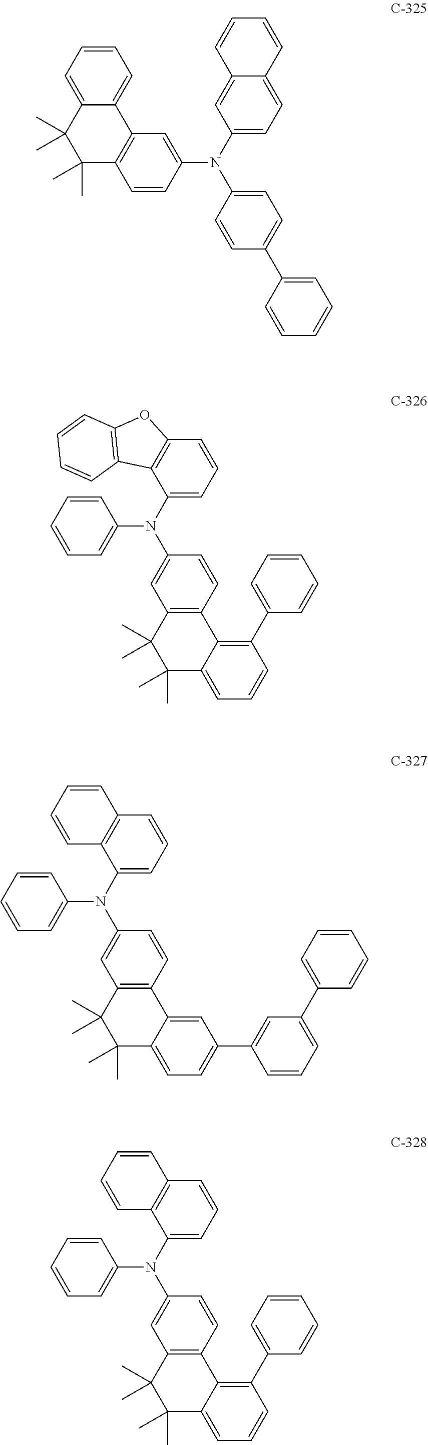 [SMILES: CC1(C)c2ccccc2-c2cc(N(c3ccc(-c4ccccc4)cc3)c3ccc4ccccc4c3)ccc2C1(C)C ; CC1(C)c2cc(N(c3ccccc3)c3cccc4oc5ccccc5c34)ccc2-c2c(-c3ccccc3)cccc2C1(C)C ; CC1(C)c2ccc(-c3cccc(-c4ccccc4)c3)cc2-c2ccc(N(c3ccccc3)c3cccc4ccccc34)cc2C1(C)C ; CC1(C)c2cc(N(c3ccccc3)c3cccc4ccccc34)ccc2-c2c(-c3ccccc3)cccc2C1(C)C]

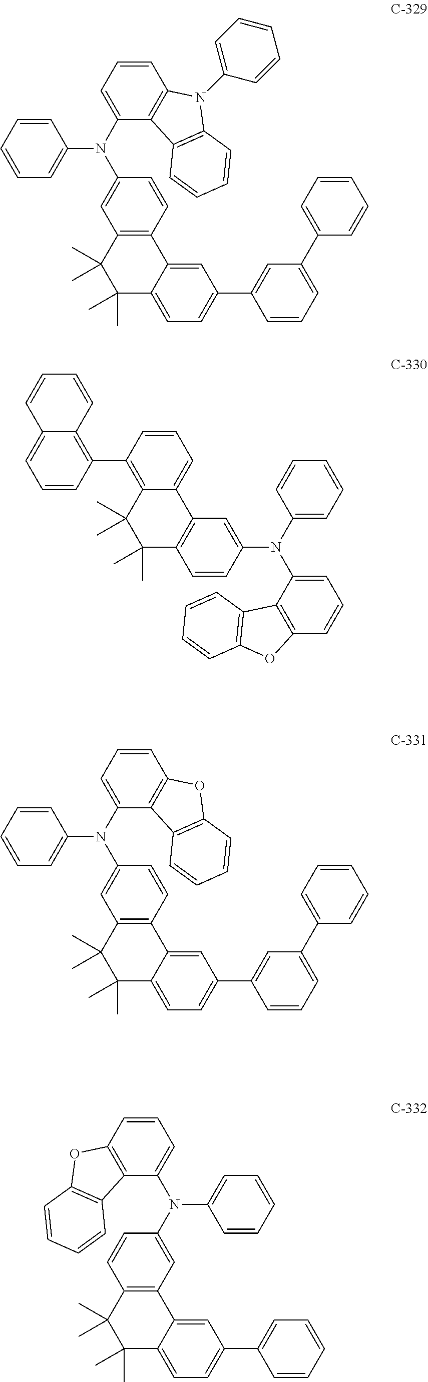 [SMILES: CC1(C)c2ccc(-c3cccc(-c4ccccc4)c3)cc2-c2ccc(N(c3ccccc3)c3cccc4c3c3ccccc3n4-c3ccccc3)cc2C1(C)C ; CC1(C)c2ccc(N(c3ccccc3)c3cccc4oc5ccccc5c34)cc2-c2cccc(-c3cccc4ccccc34)c2C1(C)C ; CC1(C)c2ccc(-c3cccc(-c4ccccc4)c3)cc2-c2ccc(N(c3ccccc3)c3cccc4oc5ccccc5c34)cc2C1(C)C ; CC1(C)c2ccc(-c3ccccc3)cc2-c2cc(N(c3ccccc3)c3cccc4oc5ccccc5c34)ccc2C1(C)C]

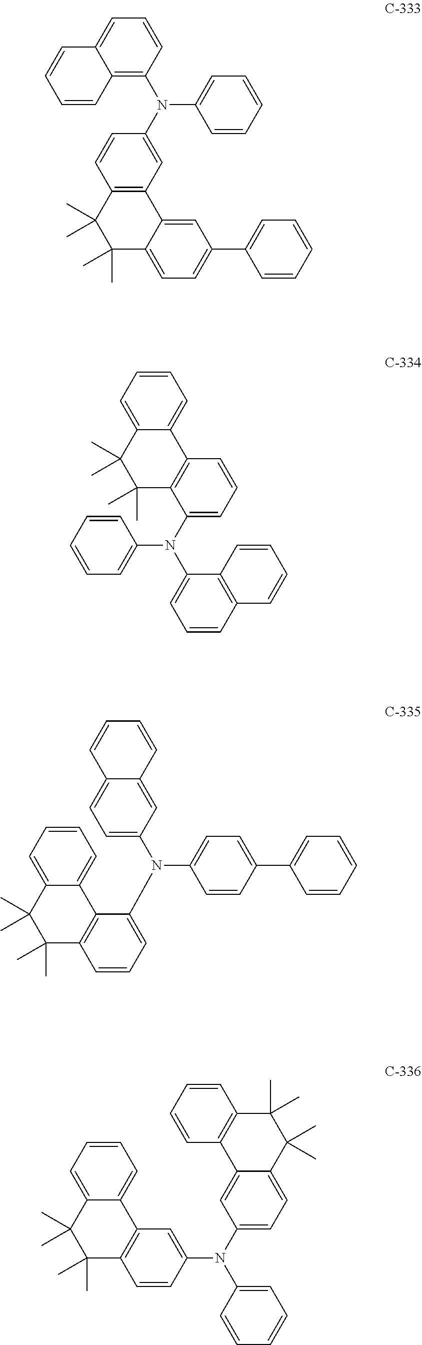

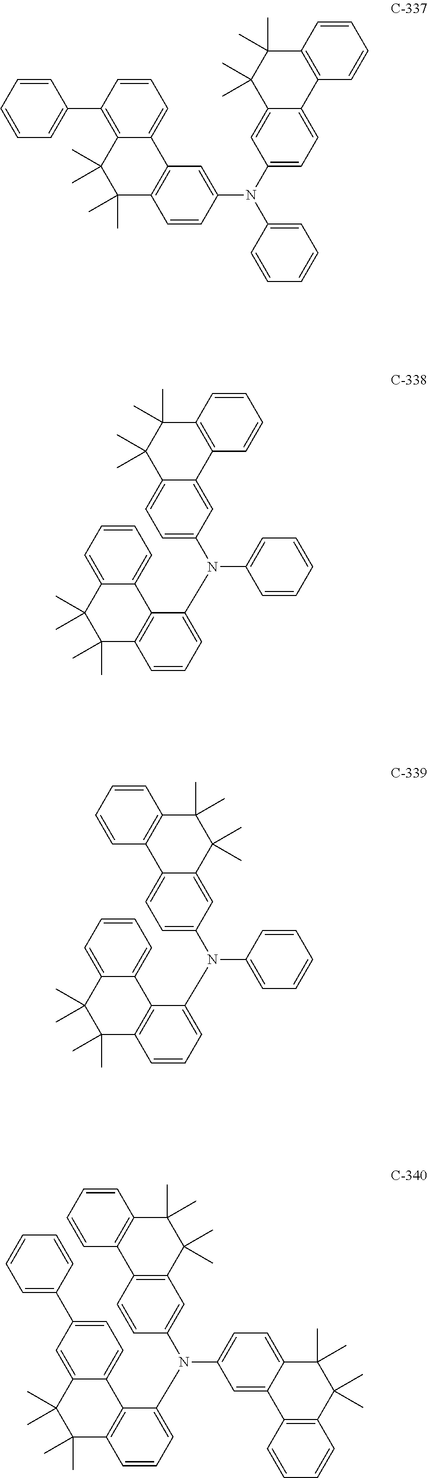 [SMILES: CC1(C)c2ccccc2-c2ccc(N(c3ccccc3)c3ccc4c(c3)-c3cccc(-c5ccccc5)c3C(C)(C)C4(C)C)cc2C1(C)C ; CC1(C)c2ccccc2-c2cc(N(c3ccccc3)c3cccc4c3-c3ccccc3C(C)(C)C4(C)C)ccc2C1(C)C ; CC1(C)c2ccccc2-c2ccc(N(c3ccccc3)c3cccc4c3-c3ccccc3C(C)(C)C4(C)C)cc2C1(C)C ; CC1(C)c2ccccc2-c2cc(N(c3ccc4c(c3)C(C)(C)C(C)(C)c3ccccc3-4)c3cccc4c3-c3ccc(-c5ccccc5)cc3C(C)(C)C4(C)C)ccc2C1(C)C]

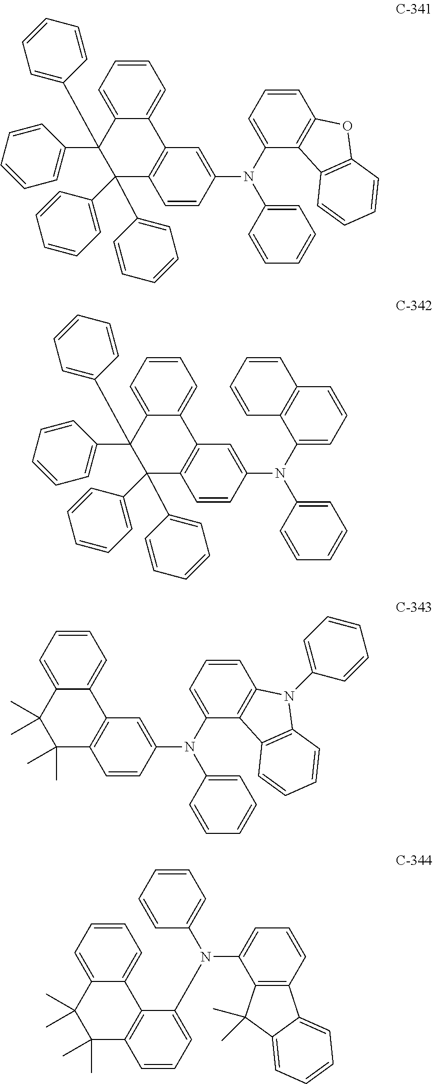

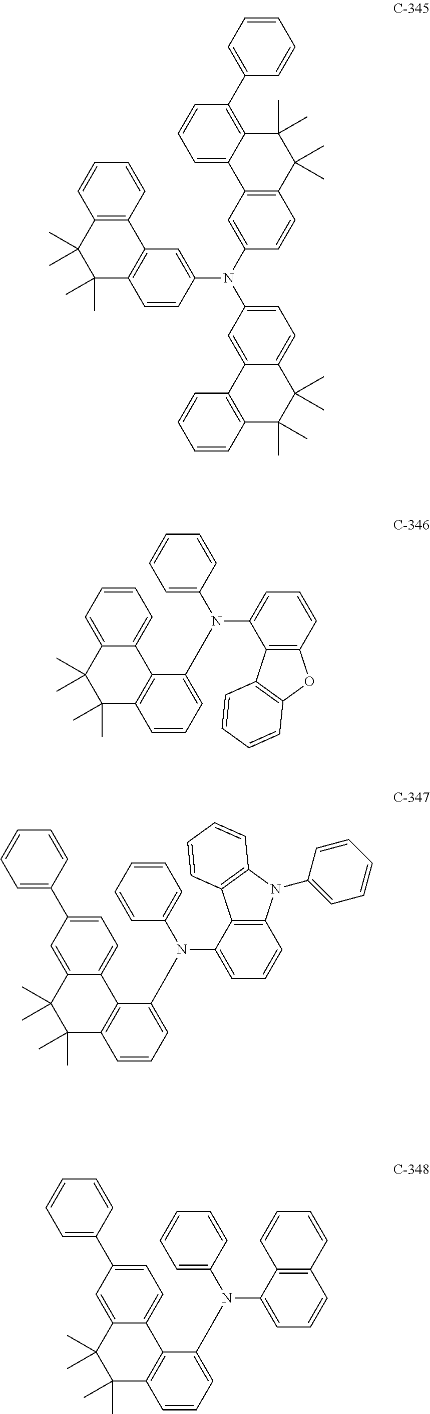

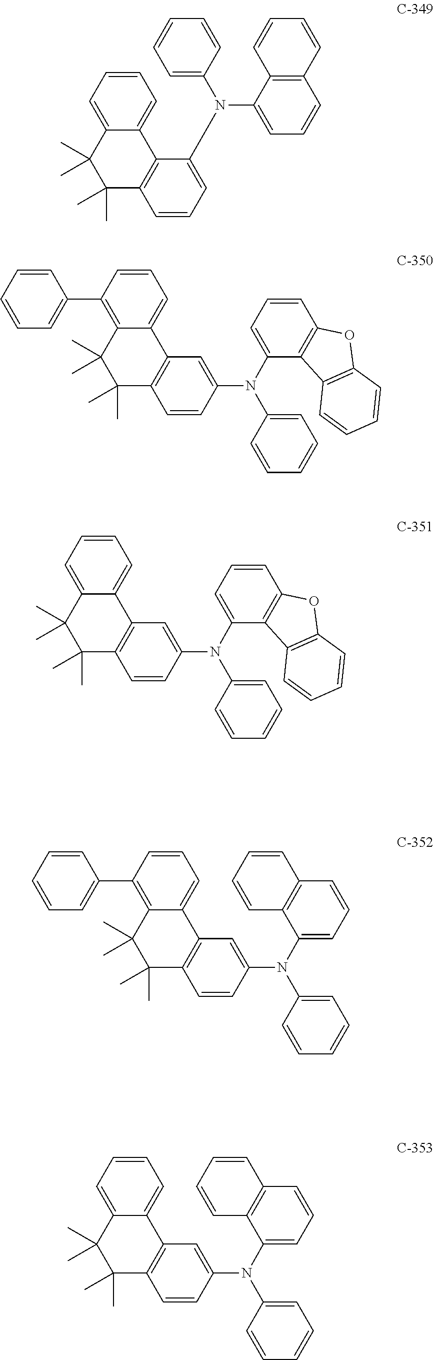 [SMILES: CC1(C)c2ccccc2-c2c(N(c3ccccc3)c3cccc4ccccc34)cccc2C1(C)C ; CC1(C)c2ccc(N(c3ccccc3)c3cccc4oc5ccccc5c34)cc2-c2cccc(-c3ccccc3)c2C1(C)C ; CC1(C)c2ccccc2-c2cc(N(c3ccccc3)c3cccc4oc5ccccc5c34)ccc2C1(C)C ; CC1(C)c2ccc(N(c3ccccc3)c3cccc4ccccc34)cc2-c2cccc(-c3ccccc3)c2C1(C)C ; CC1(C)c2ccccc2-c2cc(N(c3ccccc3)c3cccc4ccccc34)ccc2C1(C)C]

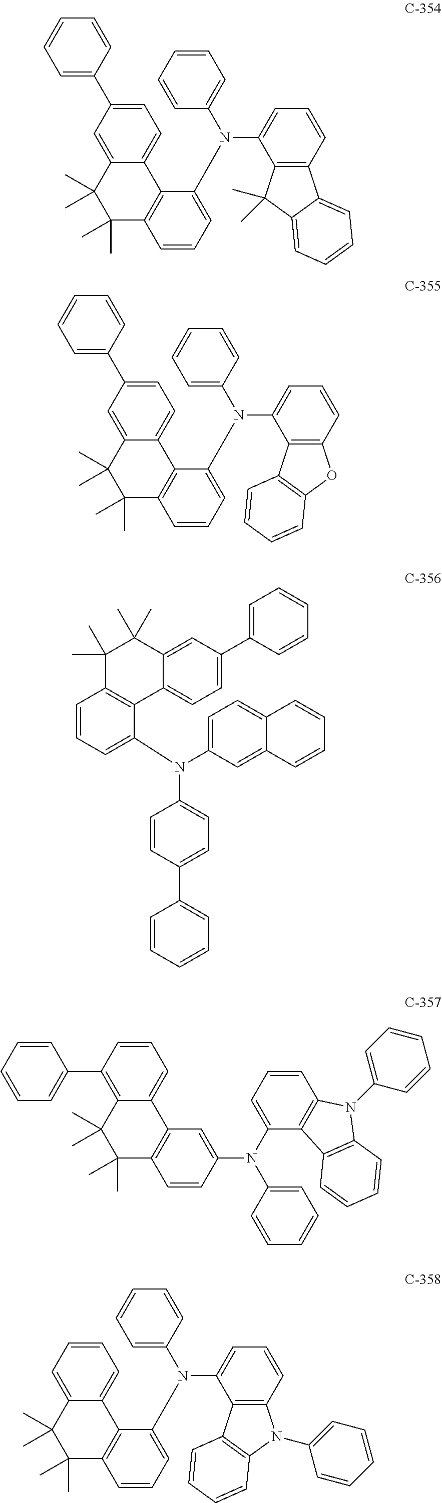

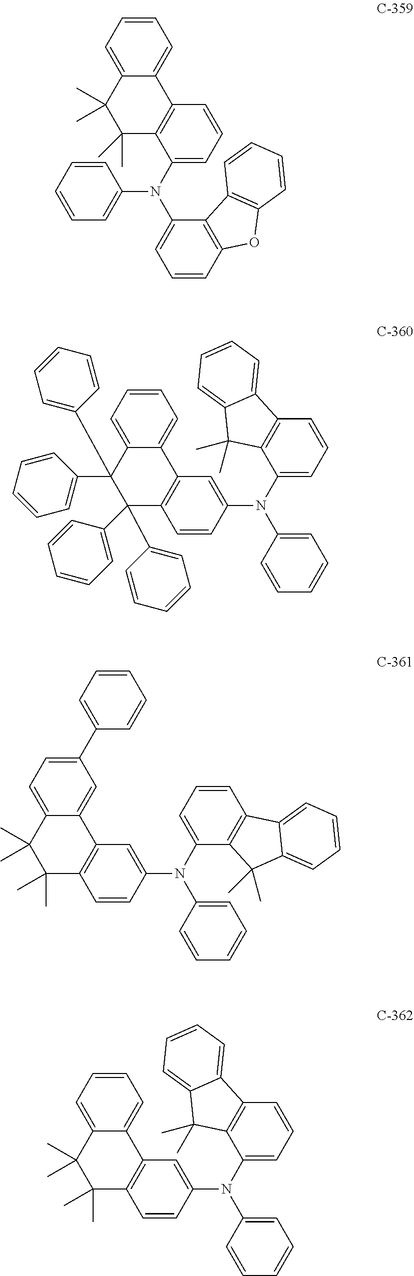 [SMILES: CC1(C)c2ccccc2-c2cccc(N(c3ccccc3)c3cccc4oc5ccccc5c34)c2C1(C)C ; CC1(C)c2ccccc2-c2cccc(N(c3ccccc3)c3ccc4c(c3)-c3ccccc3C(c3ccccc3)(c3ccccc3)C4(c3ccccc3)c3ccccc3)c21 ; CC1(C)c2ccccc2-c2cccc(N(c3ccccc3)c3ccc4c(c3)-c3cc(-c5ccccc5)ccc3C(C)(C)C4(C)C)c21 ; CC1(C)c2ccccc2-c2cccc(N(c3ccccc3)c3ccc4c(c3)-c3ccccc3C(C)(C)C4(C)C)c21]

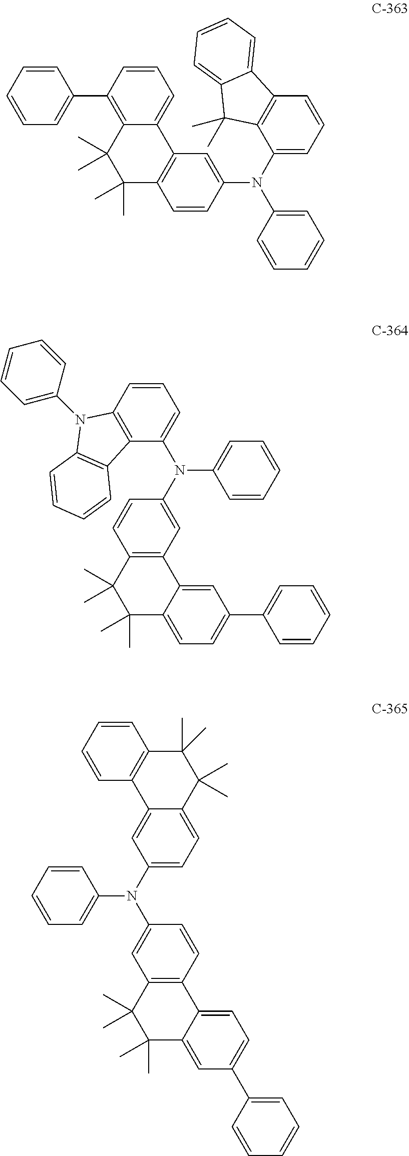 [SMILES: CC1(C)c2ccccc2-c2cccc(N(c3ccccc3)c3ccc4c(c3)-c3cccc(-c5ccccc5)c3C(C)(C)C4(C)C)c21 ; CC1(C)c2ccc(-c3ccccc3)cc2-c2cc(N(c3ccccc3)c3cccc4c3c3ccccc3n4-c3ccccc3)ccc2C1(C)C ; CC1(C)c2ccccc2-c2cc(N(c3ccccc3)c3ccc4c(c3)C(C)(C)C(C)(C)c3cc(-c5ccccc5)ccc3-4)ccc2C1(C)C]

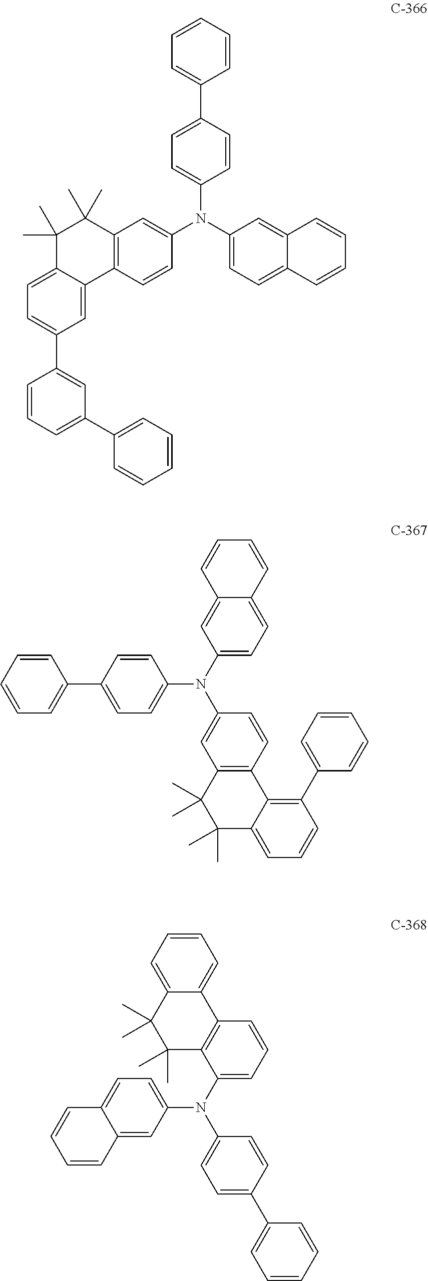 [SMILES: CC1(C)c2ccc(-c3cccc(-c4ccccc4)c3)cc2-c2ccc(N(c3ccc(-c4ccccc4)cc3)c3ccc4ccccc4c3)cc2C1(C)C ; CC1(C)c2cc(N(c3ccc(-c4ccccc4)cc3)c3ccc4ccccc4c3)ccc2-c2c(-c3ccccc3)cccc2C1(C)C ; CC1(C)c2ccccc2-c2cccc(N(c3ccc(-c4ccccc4)cc3)c3ccc4ccccc4c3)c2C1(C)C]

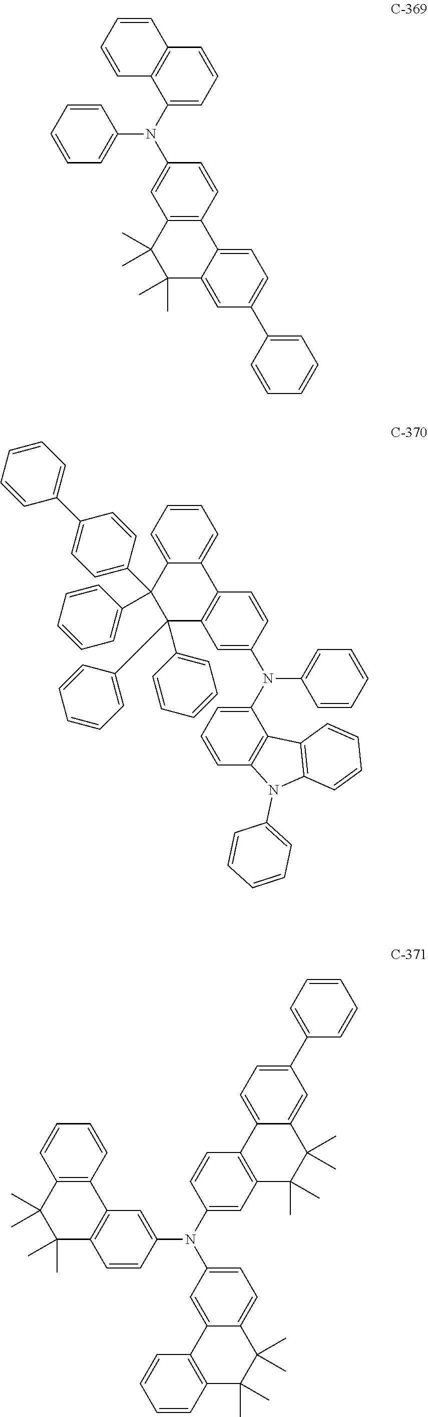 [SMILES: CC1(C)c2cc(-c3ccccc3)ccc2-c2ccc(N(c3ccccc3)c3cccc4ccccc34)cc2C1(C)C ; c1ccc(-c2ccc(C3(c4ccccc4)c4ccccc4-c4ccc(N(c5ccccc5)c5cccc6c5c5ccccc5n6-c5ccccc5)cc4C3(c3ccccc3)c3ccccc3)cc2)cc1 ; CC1(C)c2ccccc2-c2cc(N(c3ccc4c(c3)-c3ccccc3C(C)(C)C4(C)C)c3ccc4c(c3)C(C)(C)C(C)(C)c3cc(-c5ccccc5)ccc3-4)ccc2C1(C)C]

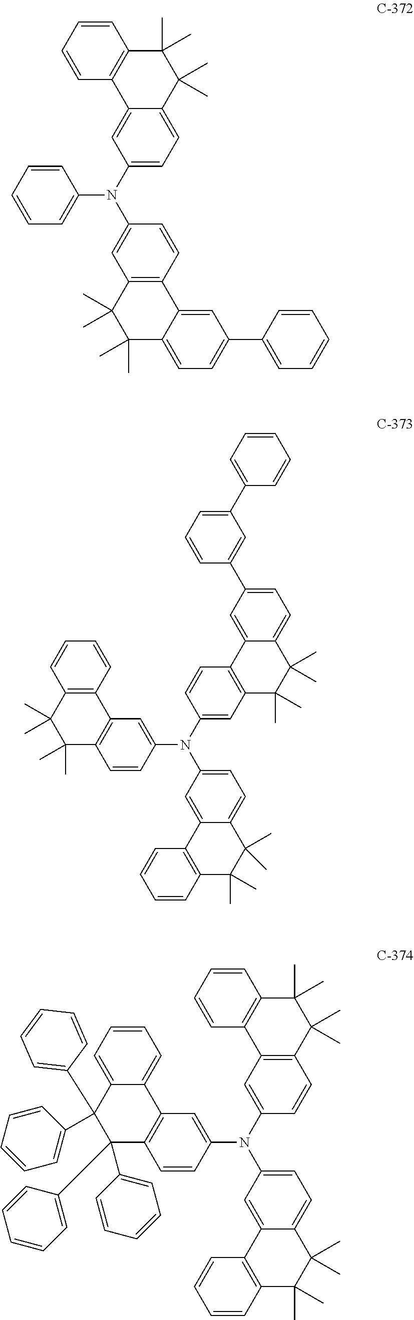

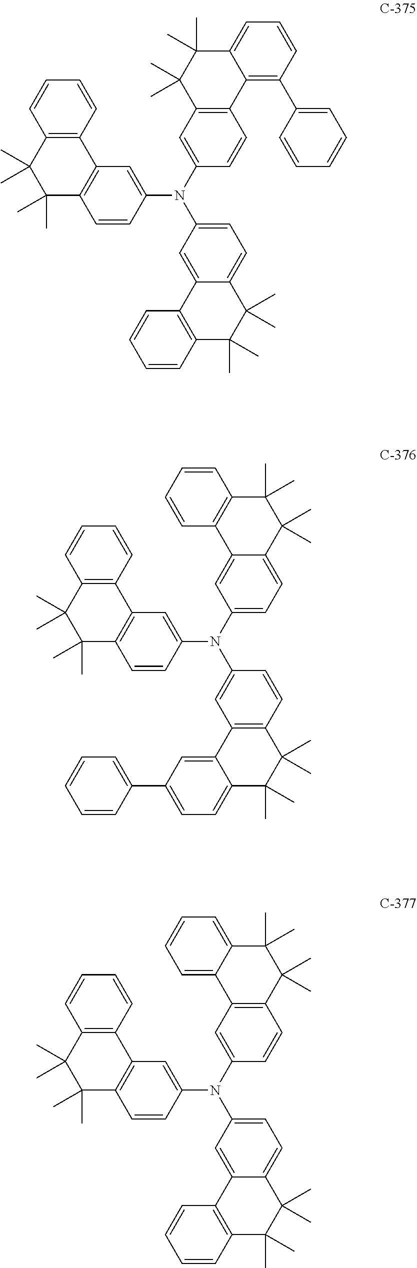

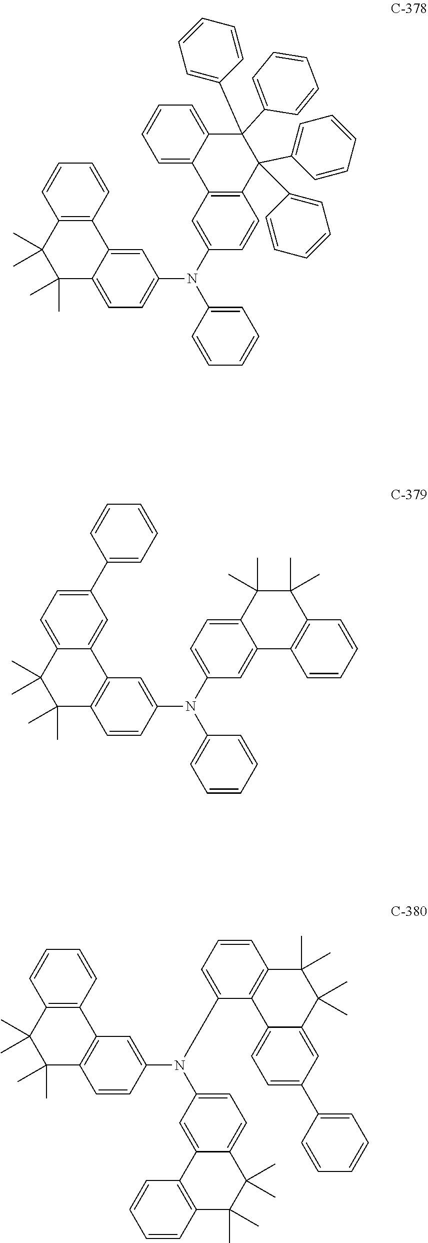 [SMILES: CC1(C)c2ccccc2-c2cc(N(c3ccccc3)c3ccc4c(c3)-c3ccccc3C(c3ccccc3)(c3ccccc3)C4(c3ccccc3)c3ccccc3)ccc2C1(C)C ; CC1(C)c2ccccc2-c2cc(N(c3ccccc3)c3ccc4c(c3)-c3cc(-c5ccccc5)ccc3C(C)(C)C4(C)C)ccc2C1(C)C ; CC1(C)c2ccccc2-c2cc(N(c3ccc4c(c3)-c3ccccc3C(C)(C)C4(C)C)c3cccc4c3-c3ccc(-c5ccccc5)cc3C(C)(C)C4(C)C)ccc2C1(C)C]

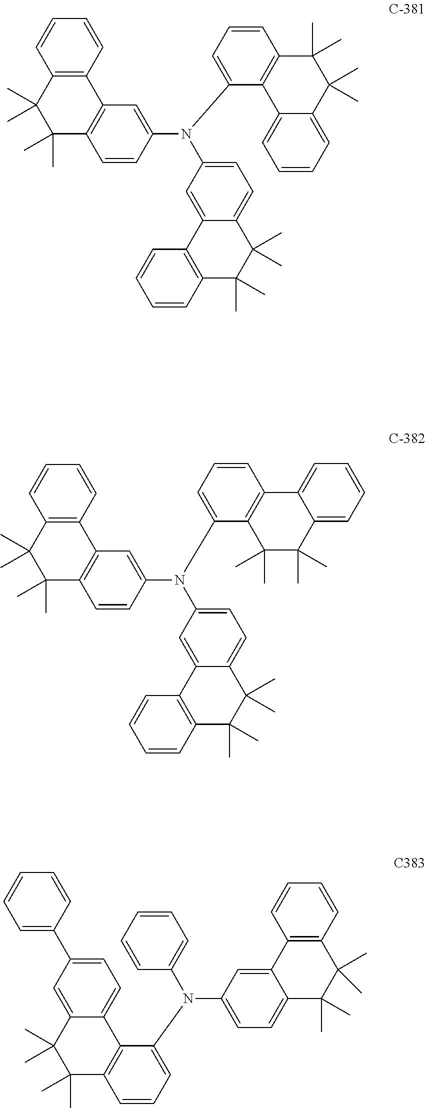

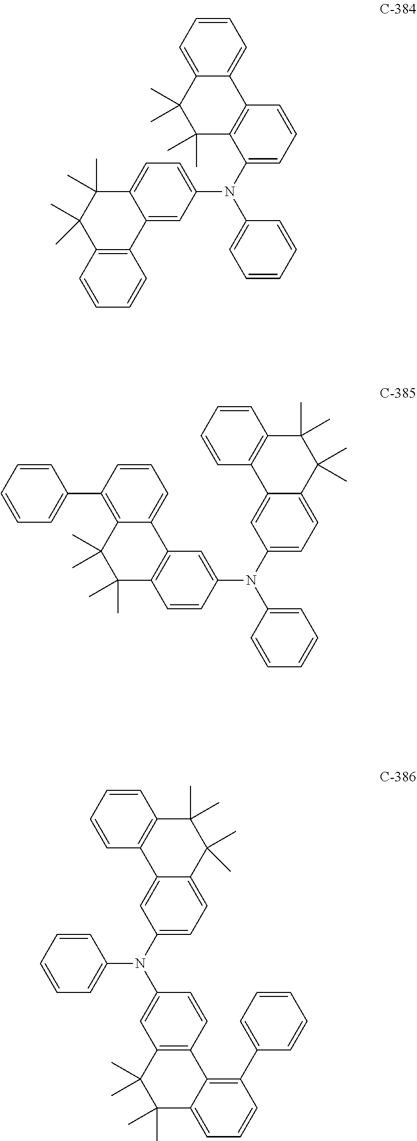 [SMILES: CC1(C)c2ccccc2-c2cc(N(c3ccccc3)c3cccc4c3C(C)(C)C(C)(C)c3ccccc3-4)ccc2C1(C)C ; CC1(C)c2ccccc2-c2cc(N(c3ccccc3)c3ccc4c(c3)-c3cccc(-c5ccccc5)c3C(C)(C)C4(C)C)ccc2C1(C)C ; CC1(C)c2ccccc2-c2cc(N(c3ccccc3)c3ccc4c(c3)C(C)(C)C(C)(C)c3cccc(-c5ccccc5)c3-4)ccc2C1(C)C]

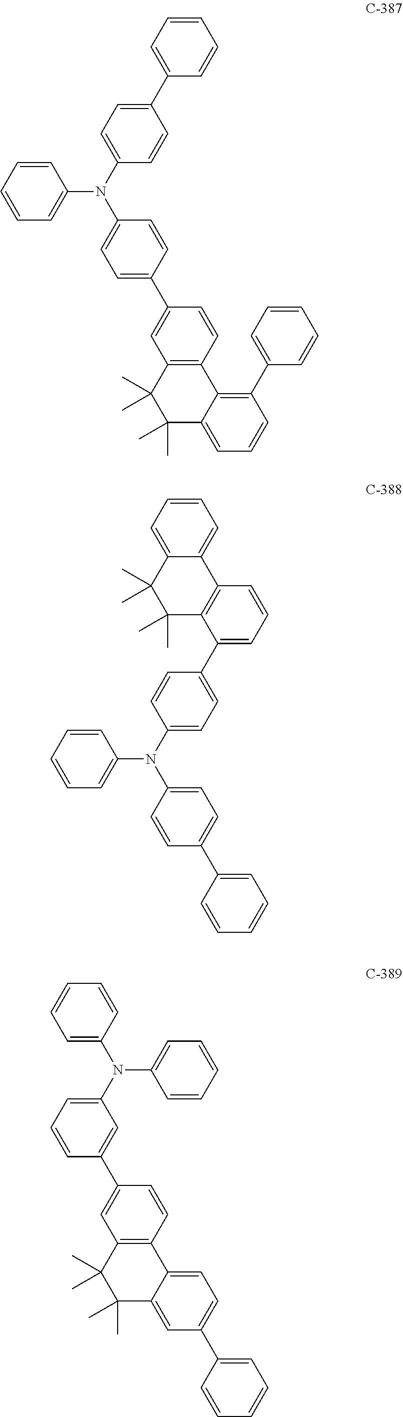

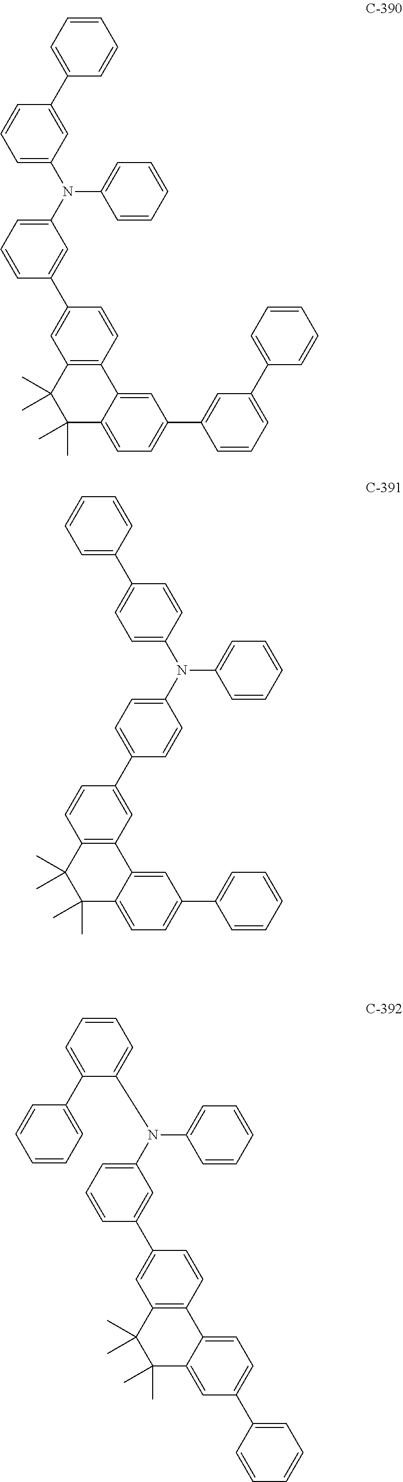

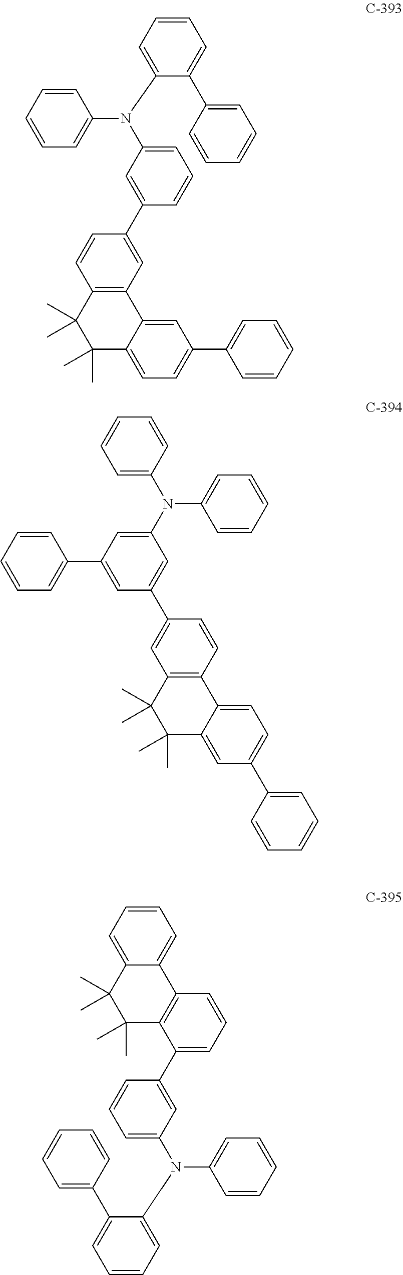

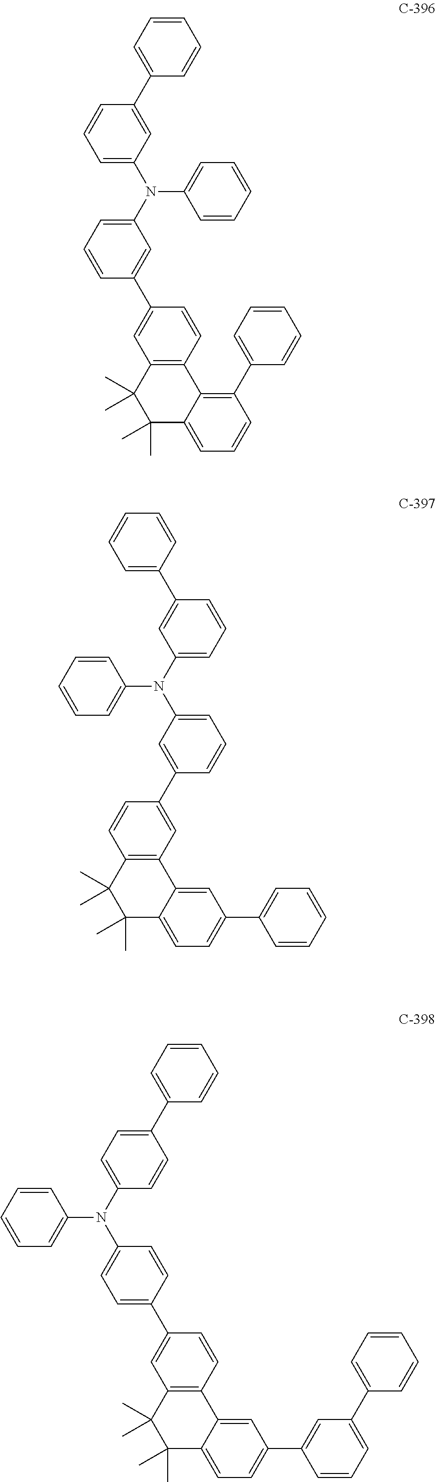 [SMILES: CC1(C)c2cc(-c3cccc(N(c4ccccc4)c4cccc(-c5ccccc5)c4)c3)ccc2-c2c(-c3ccccc3)cccc2C1(C)C ; CC1(C)c2ccc(-c3ccccc3)cc2-c2cc(-c3cccc(N(c4ccccc4)c4cccc(-c5ccccc5)c4)c3)ccc2C1(C)C ; CC1(C)c2ccc(-c3cccc(-c4ccccc4)c3)cc2-c2ccc(-c3ccc(N(c4ccccc4)c4ccc(-c5ccccc5)cc4)cc3)cc2C1(C)C]

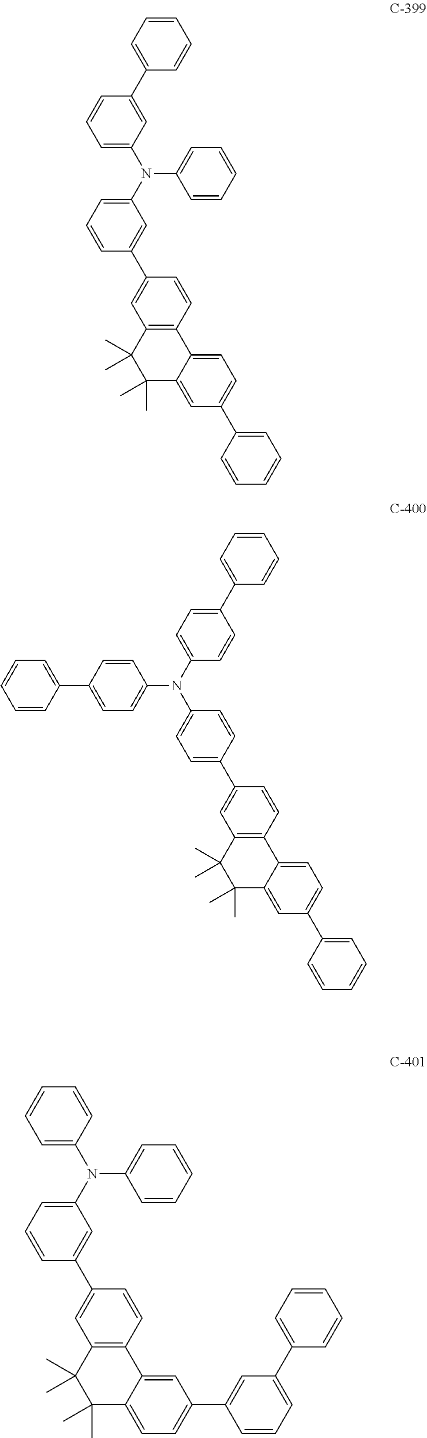 [SMILES: CC1(C)c2cc(-c3ccccc3)ccc2-c2ccc(-c3cccc(N(c4ccccc4)c4cccc(-c5ccccc5)c4)c3)cc2C1(C)C ; CC1(C)c2cc(-c3ccccc3)ccc2-c2ccc(-c3ccc(N(c4ccc(-c5ccccc5)cc4)c4ccc(-c5ccccc5)cc4)cc3)cc2C1(C)C ; CC1(C)c2ccc(-c3cccc(-c4ccccc4)c3)cc2-c2ccc(-c3cccc(N(c4ccccc4)c4ccccc4)c3)cc2C1(C)C]

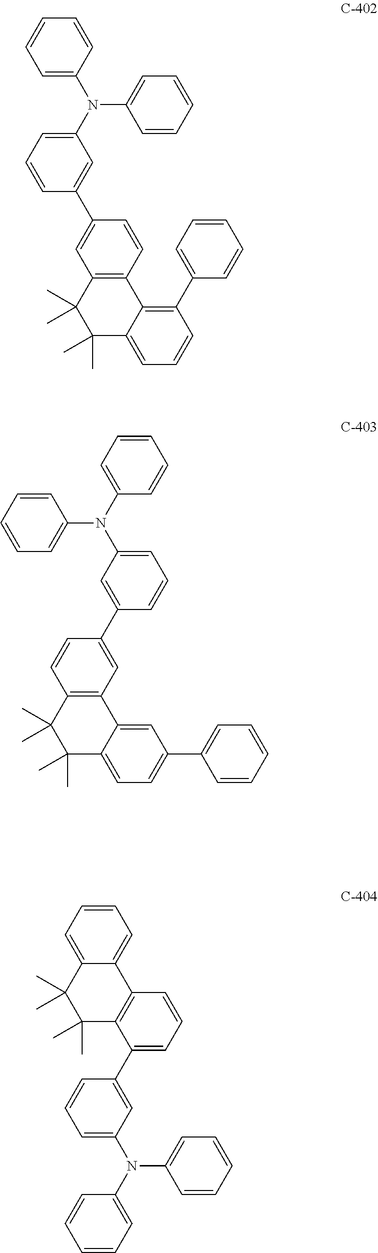 [SMILES: CC1(C)c2cc(-c3cccc(N(c4ccccc4)c4ccccc4)c3)ccc2-c2c(-c3ccccc3)cccc2C1(C)C ; CC1(C)c2ccc(-c3ccccc3)cc2-c2cc(-c3cccc(N(c4ccccc4)c4ccccc4)c3)ccc2C1(C)C ; CC1(C)c2ccccc2-c2cccc(-c3cccc(N(c4ccccc4)c4ccccc4)c3)c2C1(C)C]

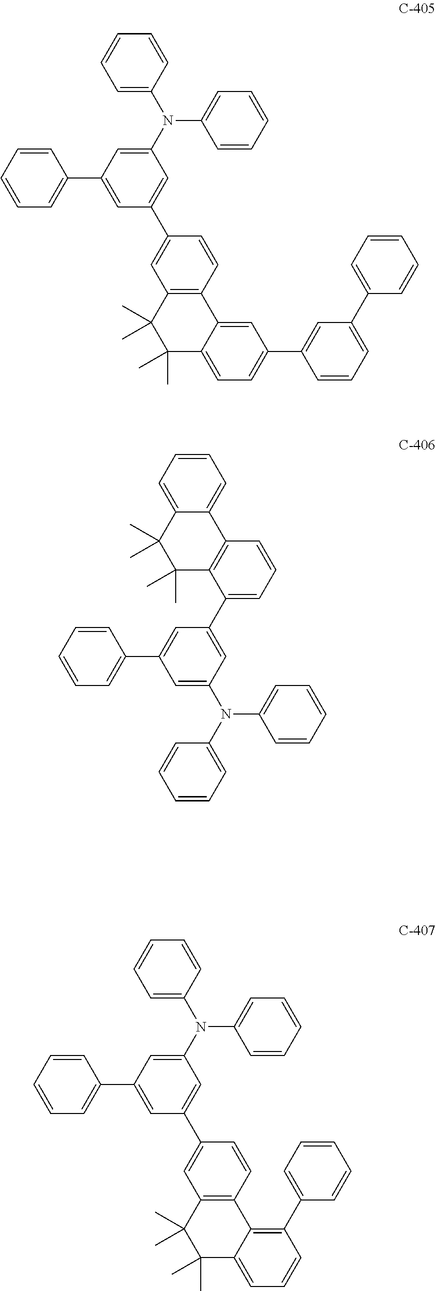 [SMILES: CC1(C)c2ccc(-c3cccc(-c4ccccc4)c3)cc2-c2ccc(-c3cc(-c4ccccc4)cc(N(c4ccccc4)c4ccccc4)c3)cc2C1(C)C ; CC1(C)c2ccccc2-c2cccc(-c3cc(-c4ccccc4)cc(N(c4ccccc4)c4ccccc4)c3)c2C1(C)C ; CC1(C)c2cc(-c3cc(-c4ccccc4)cc(N(c4ccccc4)c4ccccc4)c3)ccc2-c2c(-c3ccccc3)cccc2C1(C)C]

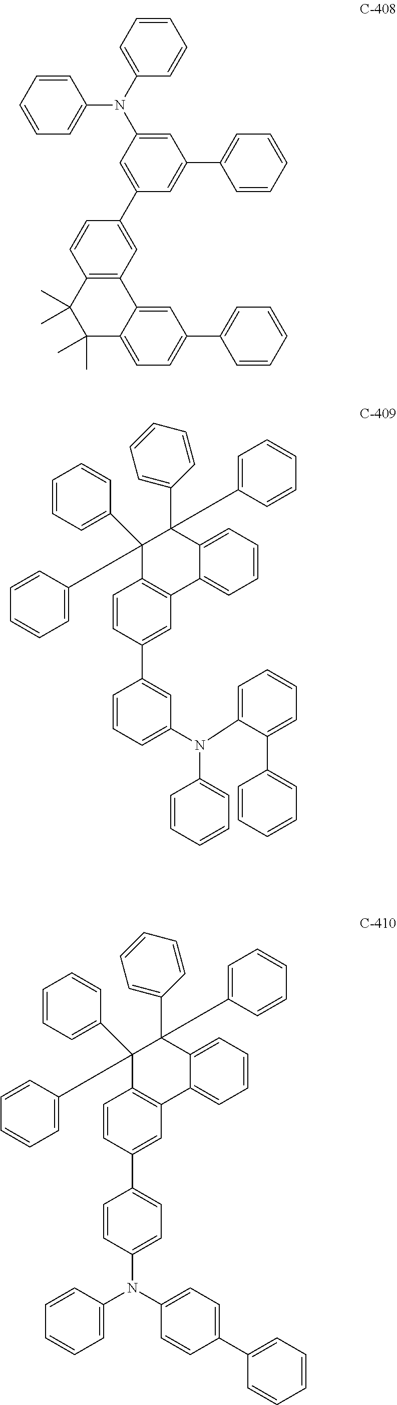 [SMILES: CC1(C)c2ccc(-c3ccccc3)cc2-c2cc(-c3cc(-c4ccccc4)cc(N(c4ccccc4)c4ccccc4)c3)ccc2C1(C)C ; c1ccc(-c2ccccc2N(c2ccccc2)c2cccc(-c3ccc4c(c3)-c3ccccc3C(c3ccccc3)(c3ccccc3)C4(c3ccccc3)c3ccccc3)c2)cc1 ; c1ccc(-c2ccc(N(c3ccccc3)c3ccc(-c4ccc5c(c4)-c4ccccc4C(c4ccccc4)(c4ccccc4)C5(c4ccccc4)c4ccccc4)cc3)cc2)cc1]

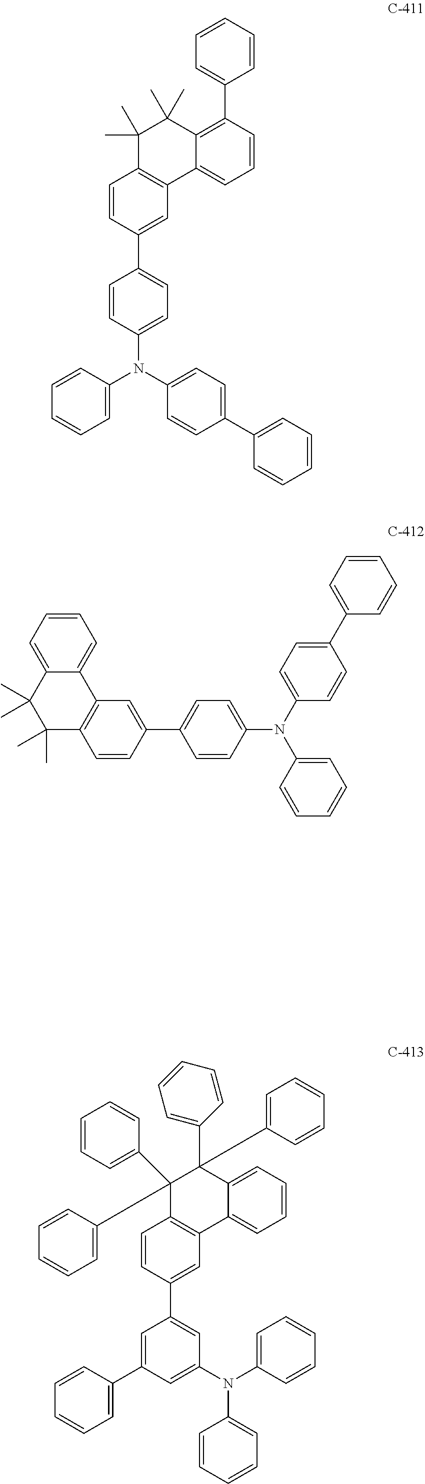 [SMILES: CC1(C)c2ccc(-c3ccc(N(c4ccccc4)c4ccc(-c5ccccc5)cc4)cc3)cc2-c2cccc(-c3ccccc3)c2C1(C)C ; CC1(C)c2ccccc2-c2cc(-c3ccc(N(c4ccccc4)c4ccc(-c5ccccc5)cc4)cc3)ccc2C1(C)C ; c1ccc(-c2cc(-c3ccc4c(c3)-c3ccccc3C(c3ccccc3)(c3ccccc3)C4(c3ccccc3)c3ccccc3)cc(N(c3ccccc3)c3ccccc3)c2)cc1]

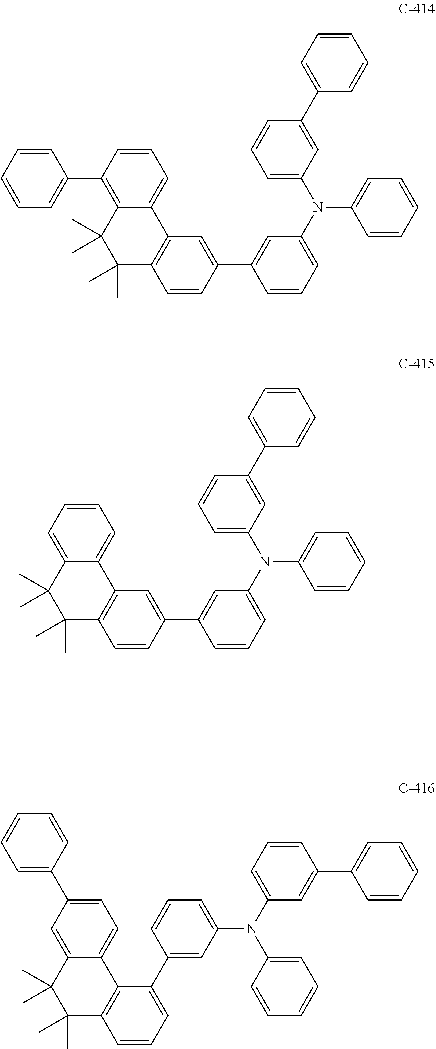 [SMILES: CC1(C)c2ccc(-c3cccc(N(c4ccccc4)c4cccc(-c5ccccc5)c4)c3)cc2-c2cccc(-c3ccccc3)c2C1(C)C ; CC1(C)c2ccccc2-c2cc(-c3cccc(N(c4ccccc4)c4cccc(-c5ccccc5)c4)c3)ccc2C1(C)C ; CC1(C)c2cc(-c3ccccc3)ccc2-c2c(-c3cccc(N(c4ccccc4)c4cccc(-c5ccccc5)c4)c3)cccc2C1(C)C]

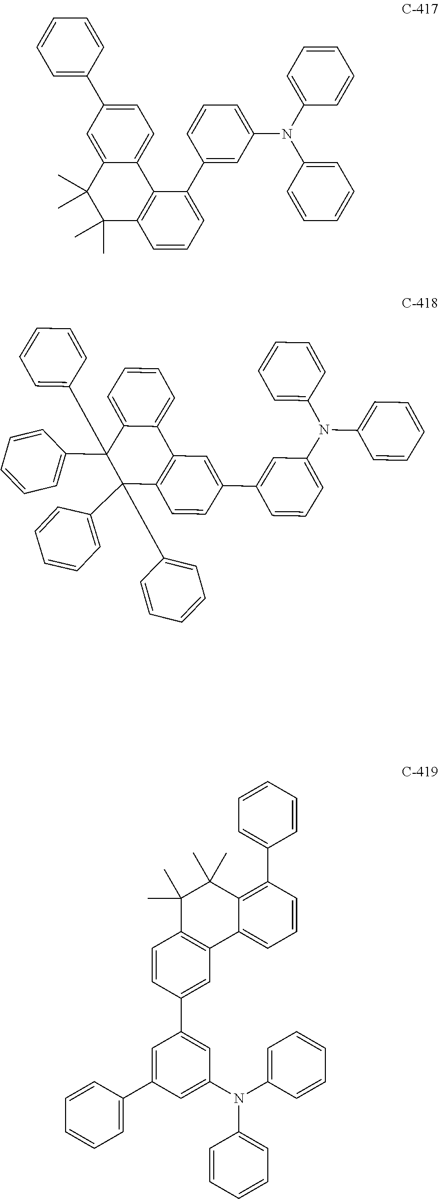 [SMILES: CC1(C)c2cc(-c3ccccc3)ccc2-c2c(-c3cccc(N(c4ccccc4)c4ccccc4)c3)cccc2C1(C)C ; c1ccc(N(c2ccccc2)c2cccc(-c3ccc4c(c3)-c3ccccc3C(c3ccccc3)(c3ccccc3)C4(c3ccccc3)c3ccccc3)c2)cc1 ; CC1(C)c2ccc(-c3cc(-c4ccccc4)cc(N(c4ccccc4)c4ccccc4)c3)cc2-c2cccc(-c3ccccc3)c2C1(C)C]

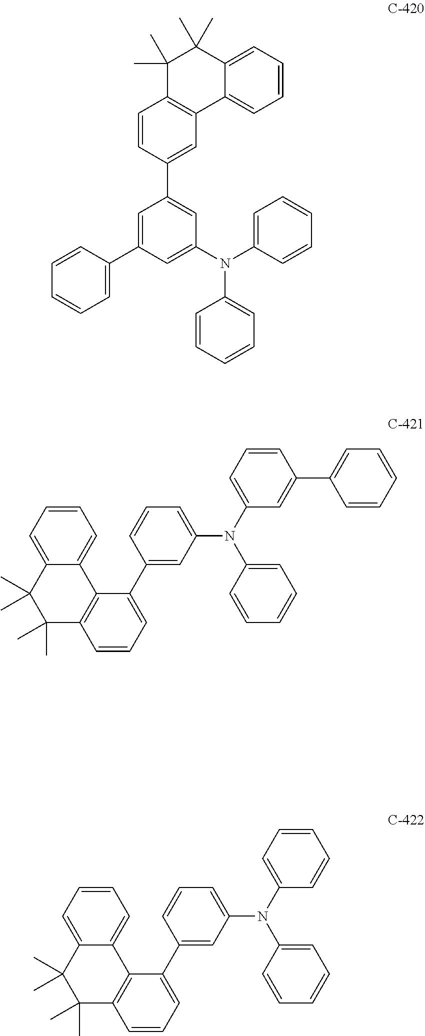 [SMILES: CC1(C)c2ccccc2-c2cc(-c3cc(-c4ccccc4)cc(N(c4ccccc4)c4ccccc4)c3)ccc2C1(C)C ; CC1(C)c2ccccc2-c2c(-c3cccc(N(c4ccccc4)c4cccc(-c5ccccc5)c4)c3)cccc2C1(C)C ; CC1(C)c2ccccc2-c2c(-c3cccc(N(c4ccccc4)c4ccccc4)c3)cccc2C1(C)C]

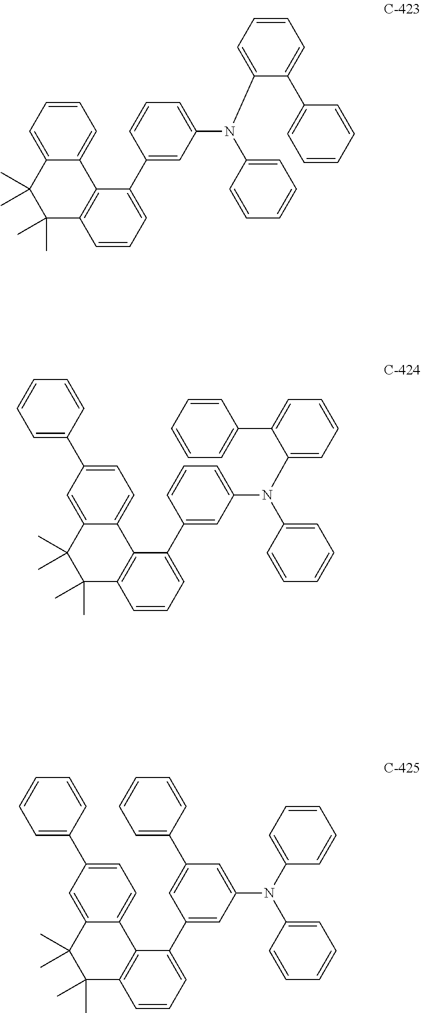 [SMILES: CC1(C)c2ccccc2-c2c(-c3cccc(N(c4ccccc4)c4ccccc4-c4ccccc4)c3)cccc2C1(C)C ; CC1(C)c2cc(-c3ccccc3)ccc2-c2c(-c3cccc(N(c4ccccc4)c4ccccc4-c4ccccc4)c3)cccc2C1(C)C ; CC1(C)c2cc(-c3ccccc3)ccc2-c2c(-c3cc(-c4ccccc4)cc(N(c4ccccc4)c4ccccc4)c3)cccc2C1(C)C]

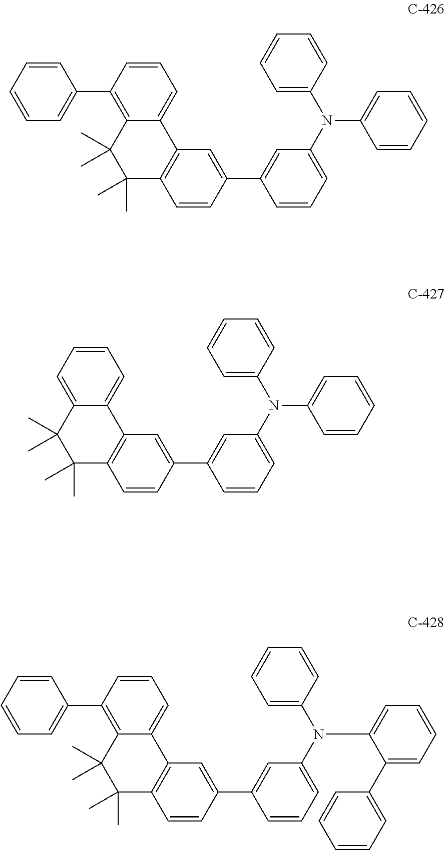 [SMILES: CC1(C)c2ccc(-c3cccc(N(c4ccccc4)c4ccccc4)c3)cc2-c2cccc(-c3ccccc3)c2C1(C)C ; CC1(C)c2ccccc2-c2cc(-c3cccc(N(c4ccccc4)c4ccccc4)c3)ccc2C1(C)C ; CC1(C)c2ccc(-c3cccc(N(c4ccccc4)c4ccccc4-c4ccccc4)c3)cc2-c2cccc(-c3ccccc3)c2C1(C)C]

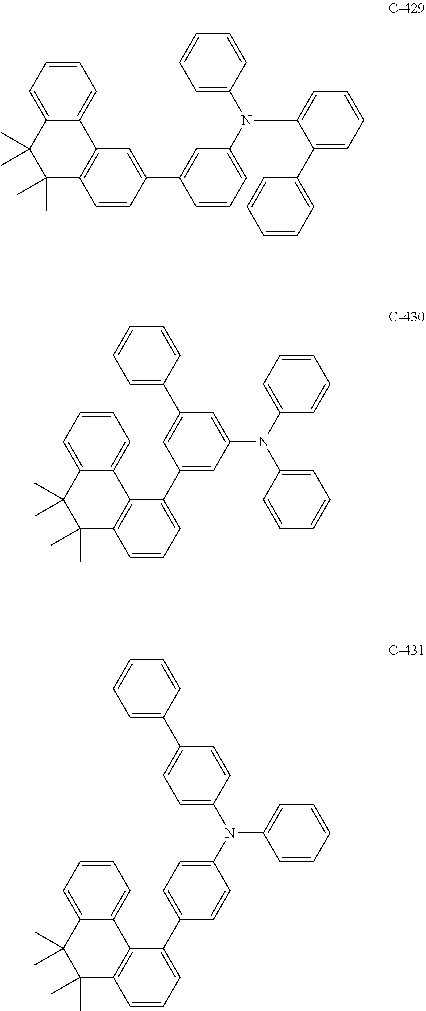 [SMILES: CC1(C)c2ccccc2-c2cc(-c3cccc(N(c4ccccc4)c4ccccc4-c4ccccc4)c3)ccc2C1(C)C ; CC1(C)c2ccccc2-c2c(-c3cc(-c4ccccc4)cc(N(c4ccccc4)c4ccccc4)c3)cccc2C1(C)C ; CC1(C)c2ccccc2-c2c(-c3ccc(N(c4ccccc4)c4ccc(-c5ccccc5)cc4)cc3)cccc2C1(C)C]

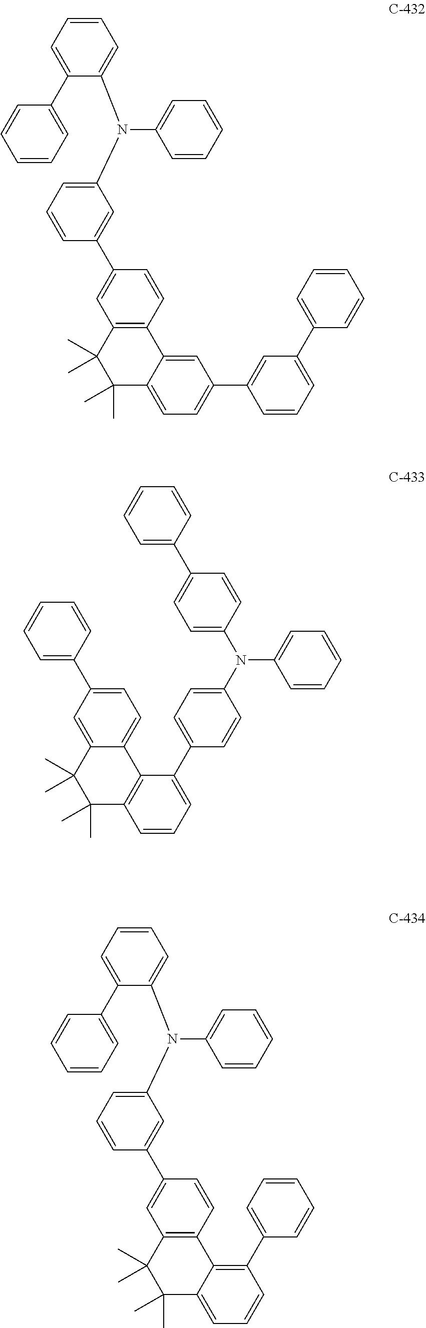 [SMILES: CC1(C)c2ccc(-c3cccc(-c4ccccc4)c3)cc2-c2ccc(-c3cccc(N(c4ccccc4)c4ccccc4-c4ccccc4)c3)cc2C1(C)C ; CC1(C)c2cc(-c3ccccc3)ccc2-c2c(-c3ccc(N(c4ccccc4)c4ccc(-c5ccccc5)cc4)cc3)cccc2C1(C)C ; CC1(C)c2cc(-c3cccc(N(c4ccccc4)c4ccccc4-c4ccccc4)c3)ccc2-c2c(-c3ccccc3)cccc2C1(C)C]

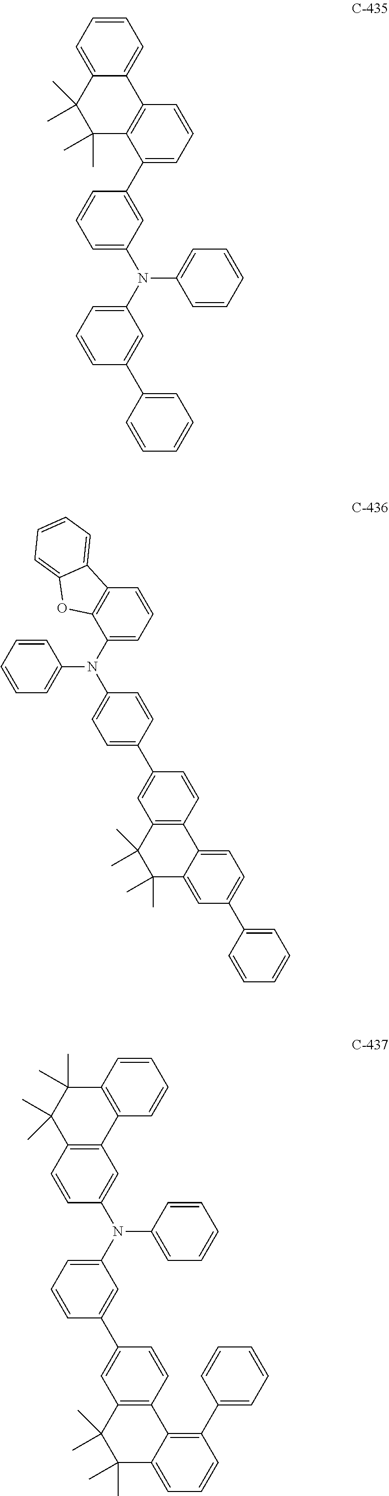 [SMILES: CC1(C)c2ccccc2-c2cccc(-c3cccc(N(c4ccccc4)c4cccc(-c5ccccc5)c4)c3)c2C1(C)C ; CC1(C)c2cc(-c3ccccc3)ccc2-c2ccc(-c3ccc(N(c4ccccc4)c4cccc5c4oc4ccccc45)cc3)cc2C1(C)C ; CC1(C)c2ccccc2-c2cc(N(c3ccccc3)c3cccc(-c4ccc5c(c4)C(C)(C)C(C)(C)c4cccc(-c6ccccc6)c4-5)c3)ccc2C1(C)C]

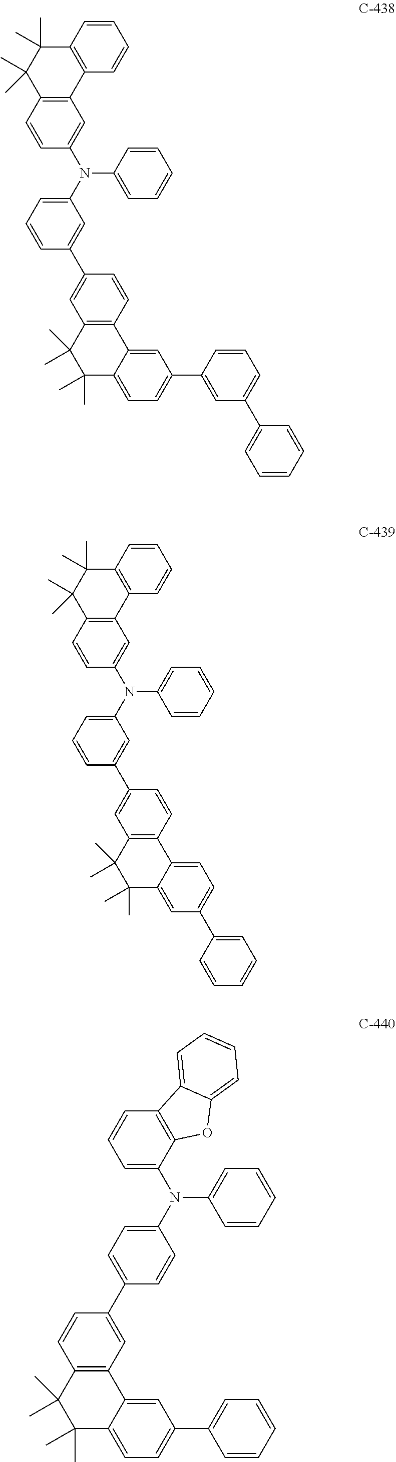 [SMILES: CC1(C)c2ccccc2-c2cc(N(c3ccccc3)c3cccc(-c4ccc5c(c4)C(C)(C)C(C)(C)c4ccc(-c6cccc(-c7ccccc7)c6)cc4-5)c3)ccc2C1(C)C ; CC1(C)c2ccccc2-c2cc(N(c3ccccc3)c3cccc(-c4ccc5c(c4)C(C)(C)C(C)(C)c4cc(-c6ccccc6)ccc4-5)c3)ccc2C1(C)C ; CC1(C)c2ccc(-c3ccccc3)cc2-c2cc(-c3ccc(N(c4ccccc4)c4cccc5c4oc4ccccc45)cc3)ccc2C1(C)C]

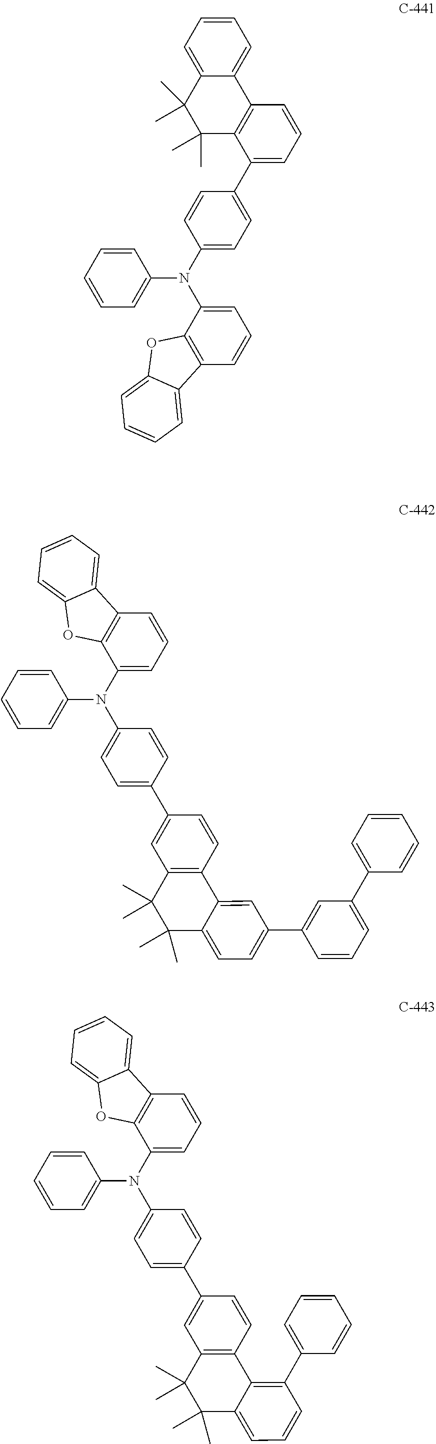 [SMILES: CC1(C)c2ccccc2-c2cccc(-c3ccc(N(c4ccccc4)c4cccc5c4oc4ccccc45)cc3)c2C1(C)C ; CC1(C)c2ccc(-c3cccc(-c4ccccc4)c3)cc2-c2ccc(-c3ccc(N(c4ccccc4)c4cccc5c4oc4ccccc45)cc3)cc2C1(C)C ; CC1(C)c2cc(-c3ccc(N(c4ccccc4)c4cccc5c4oc4ccccc45)cc3)ccc2-c2c(-c3ccccc3)cccc2C1(C)C]

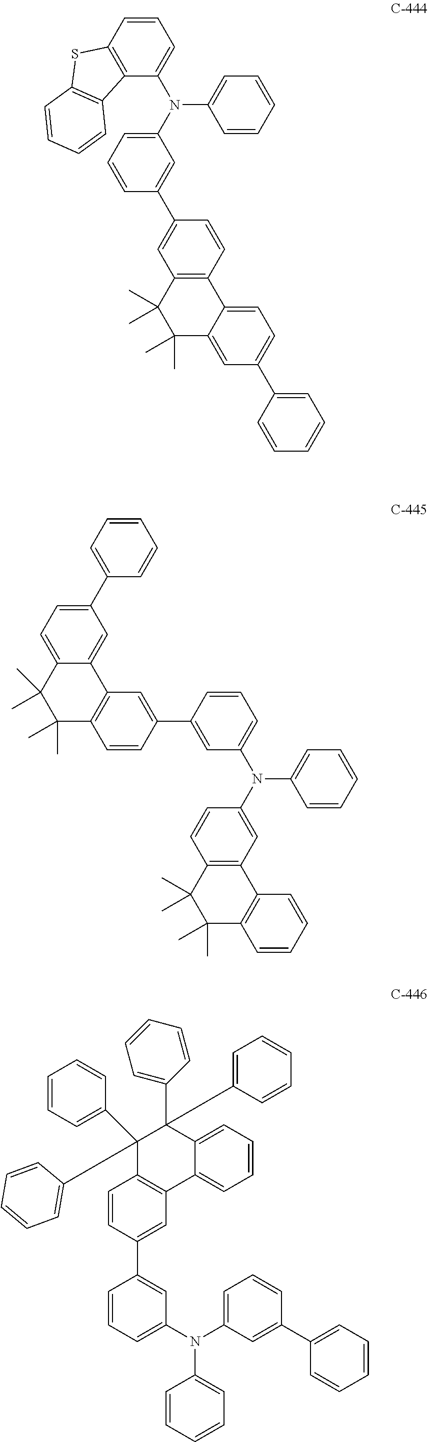 [SMILES: CC1(C)c2cc(-c3ccccc3)ccc2-c2ccc(-c3cccc(N(c4ccccc4)c4cccc5sc6ccccc6c45)c3)cc2C1(C)C ; CC1(C)c2ccccc2-c2cc(N(c3ccccc3)c3cccc(-c4ccc5c(c4)-c4cc(-c6ccccc6)ccc4C(C)(C)C5(C)C)c3)ccc2C1(C)C ; c1ccc(-c2cccc(N(c3ccccc3)c3cccc(-c4ccc5c(c4)-c4ccccc4C(c4ccccc4)(c4ccccc4)C5(c4ccccc4)c4ccccc4)c3)c2)cc1]

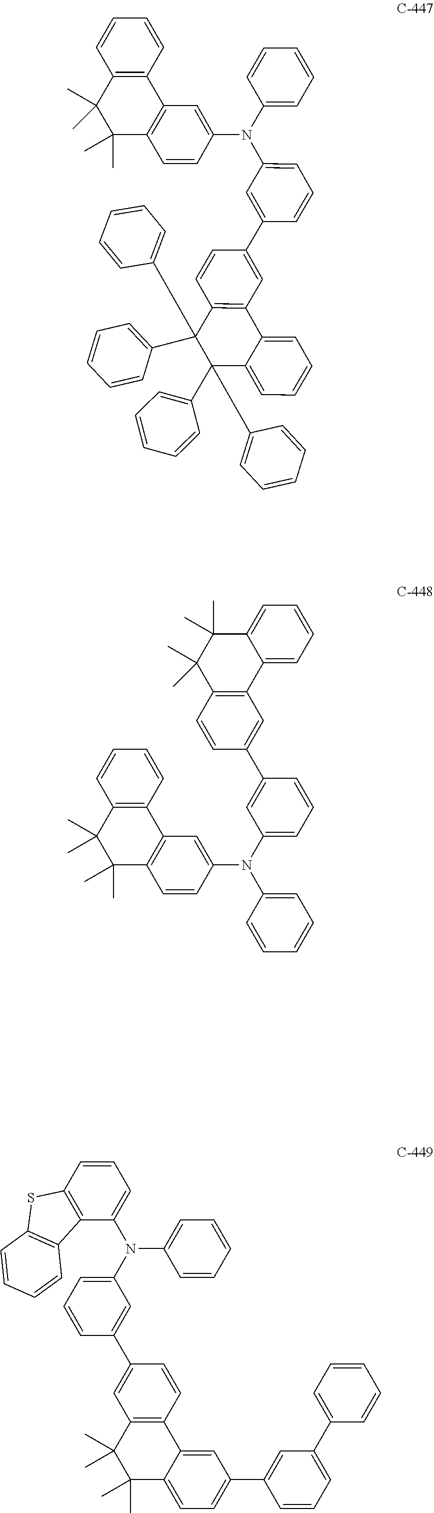 [SMILES: CC1(C)c2ccccc2-c2cc(N(c3ccccc3)c3cccc(-c4ccc5c(c4)-c4ccccc4C(c4ccccc4)(c4ccccc4)C5(c4ccccc4)c4ccccc4)c3)ccc2C1(C)C ; CC1(C)c2ccccc2-c2cc(-c3cccc(N(c4ccccc4)c4ccc5c(c4)-c4ccccc4C(C)(C)C5(C)C)c3)ccc2C1(C)C ; CC1(C)c2ccc(-c3cccc(-c4ccccc4)c3)cc2-c2ccc(-c3cccc(N(c4ccccc4)c4cccc5sc6ccccc6c45)c3)cc2C1(C)C]

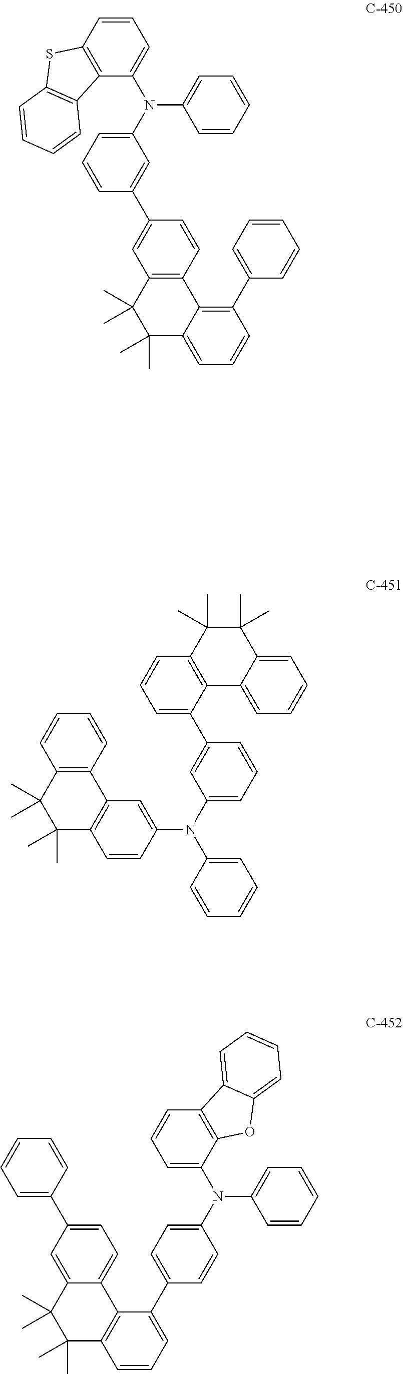 [SMILES: CC1(C)c2cc(-c3cccc(N(c4ccccc4)c4cccc5sc6ccccc6c45)c3)ccc2-c2c(-c3ccccc3)cccc2C1(C)C ; CC1(C)c2ccccc2-c2cc(N(c3ccccc3)c3cccc(-c4cccc5c4-c4ccccc4C(C)(C)C5(C)C)c3)ccc2C1(C)C ; CC1(C)c2cc(-c3ccccc3)ccc2-c2c(-c3ccc(N(c4ccccc4)c4cccc5c4oc4ccccc45)cc3)cccc2C1(C)C]

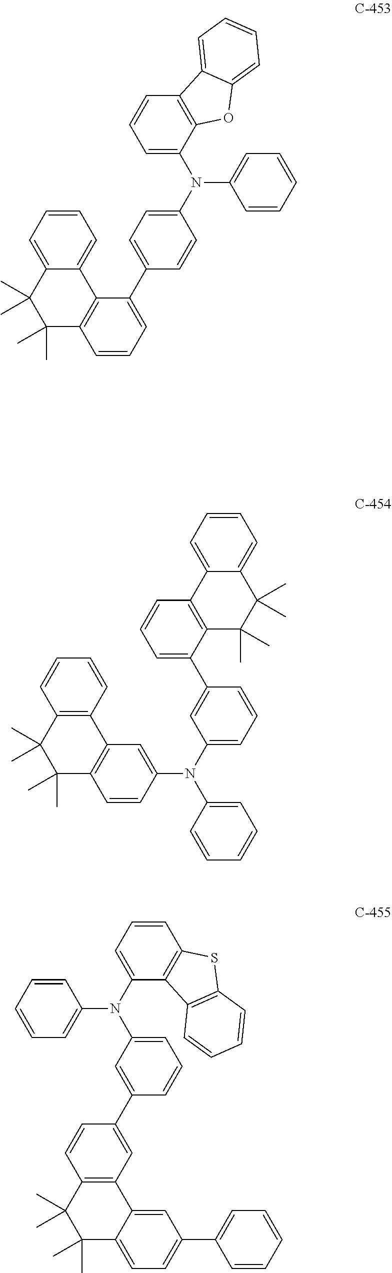

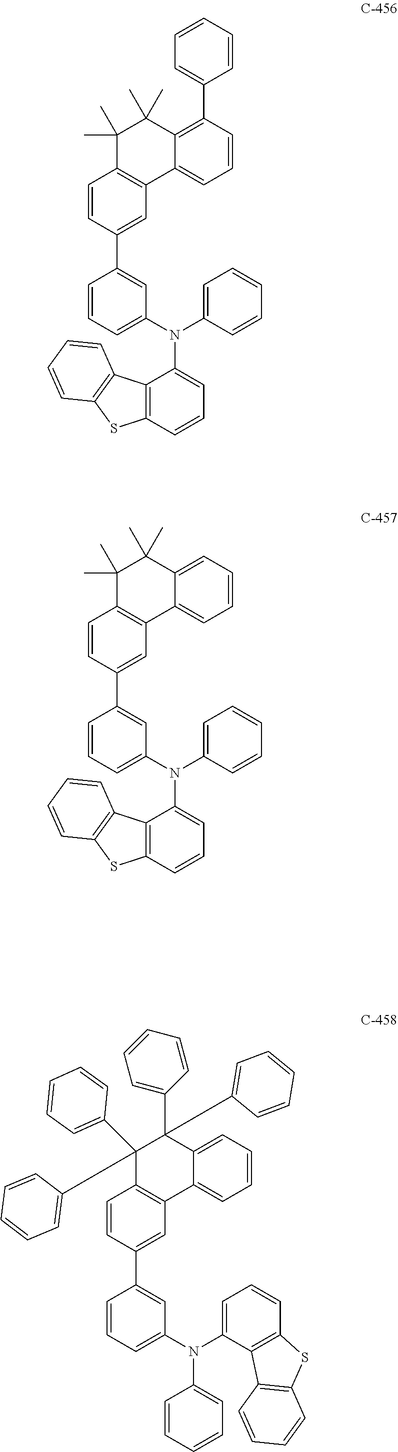 [SMILES: CC1(C)c2ccc(-c3cccc(N(c4ccccc4)c4cccc5sc6ccccc6c45)c3)cc2-c2cccc(-c3ccccc3)c2C1(C)C ; CC1(C)c2ccccc2-c2cc(-c3cccc(N(c4ccccc4)c4cccc5sc6ccccc6c45)c3)ccc2C1(C)C ; c1ccc(N(c2cccc(-c3ccc4c(c3)-c3ccccc3C(c3ccccc3)(c3ccccc3)C4(c3ccccc3)c3ccccc3)c2)c2cccc3sc4ccccc4c23)cc1]

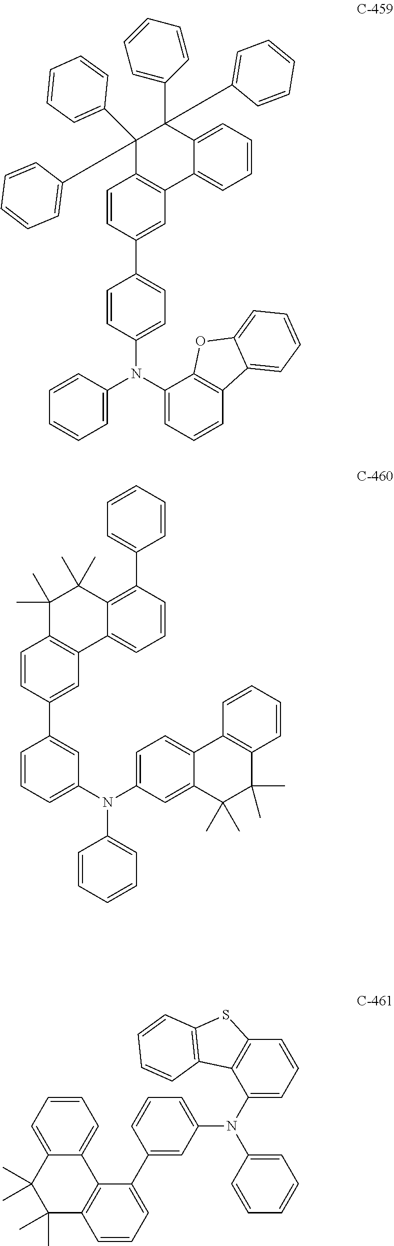

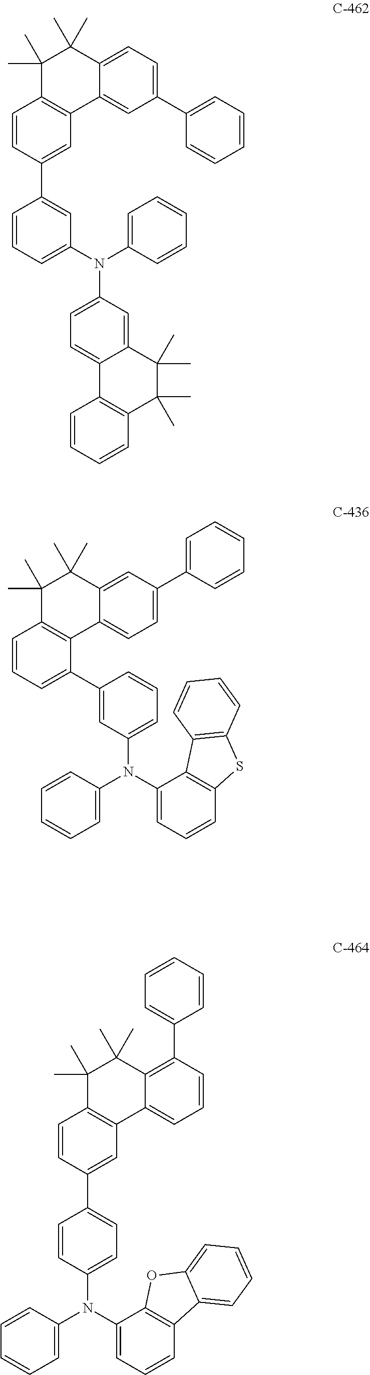 [SMILES: CC1(C)c2ccc(-c3ccccc3)cc2-c2cc(-c3cccc(N(c4ccccc4)c4ccc5c(c4)C(C)(C)C(C)(C)c4ccccc4-5)c3)ccc2C1(C)C ; CC1(C)c2cc(-c3ccccc3)ccc2-c2c(-c3cccc(N(c4ccccc4)c4cccc5sc6ccccc6c45)c3)cccc2C1(C)C ; CC1(C)c2ccc(-c3ccc(N(c4ccccc4)c4cccc5c4oc4ccccc45)cc3)cc2-c2cccc(-c3ccccc3)c2C1(C)C]

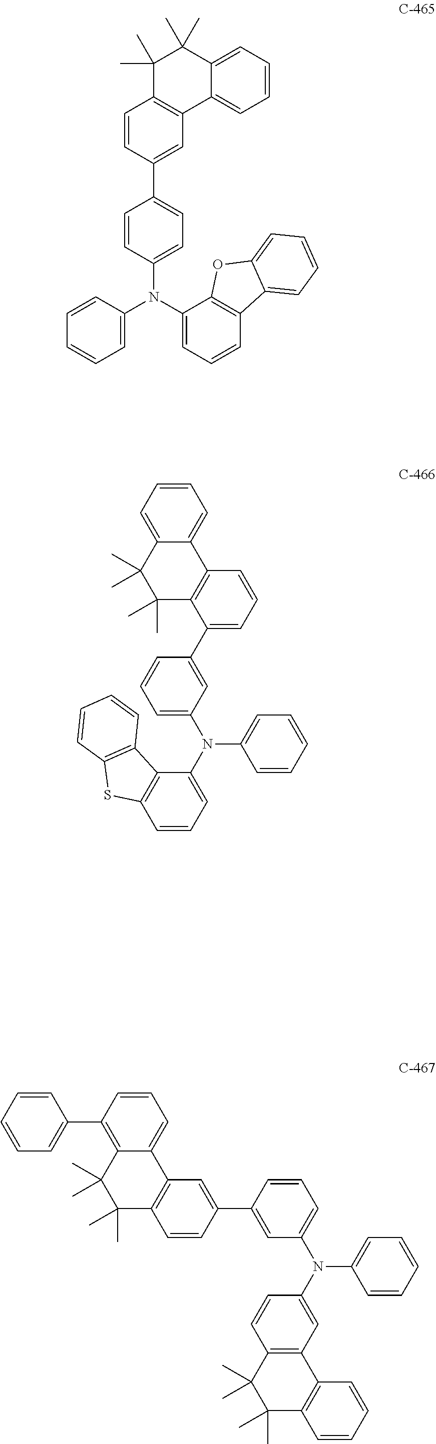 [SMILES: CC1(C)c2ccccc2-c2cc(-c3ccc(N(c4ccccc4)c4cccc5c4oc4ccccc45)cc3)ccc2C1(C)C ; CC1(C)c2ccccc2-c2cccc(-c3cccc(N(c4ccccc4)c4cccc5sc6ccccc6c45)c3)c2C1(C)C ; CC1(C)c2ccccc2-c2cc(N(c3ccccc3)c3cccc(-c4ccc5c(c4)-c4cccc(-c6ccccc6)c4C(C)(C)C5(C)C)c3)ccc2C1(C)C]

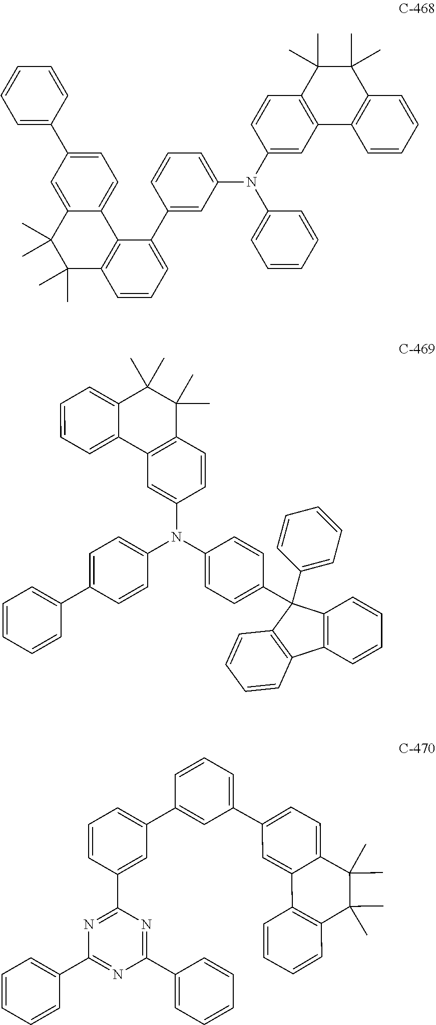 [SMILES: CC1(C)c2ccccc2-c2cc(N(c3ccccc3)c3cccc(-c4cccc5c4-c4ccc(-c6ccccc6)cc4C(C)(C)C5(C)C)c3)ccc2C1(C)C ; CC1(C)c2ccccc2-c2cc(N(c3ccc(-c4ccccc4)cc3)c3ccc(C4(c5ccccc5)c5ccccc5-c5ccccc54)cc3)ccc2C1(C)C ; CC1(C)c2ccccc2-c2cc(-c3cccc(-c4cccc(-c5nc(-c6ccccc6)nc(-c6ccccc6)n5)c4)c3)ccc2C1(C)C]

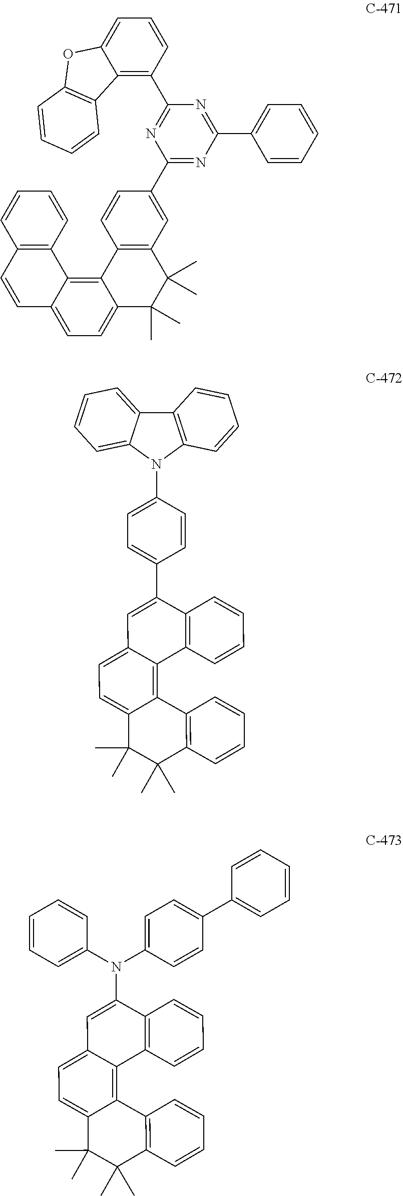 [SMILES: CC1(C)c2cc(-c3nc(-c4ccccc4)nc(-c4cccc5oc6ccccc6c45)n3)ccc2-c2c(ccc3ccc4ccccc4c23)C1(C)C ; CC1(C)c2ccccc2-c2c(ccc3cc(-c4ccc(-n5c6ccccc6c6ccccc65)cc4)c4ccccc4c23)C1(C)C ; CC1(C)c2ccccc2-c2c(ccc3cc(N(c4ccccc4)c4ccc(-c5ccccc5)cc4)c4ccccc4c23)C1(C)C]

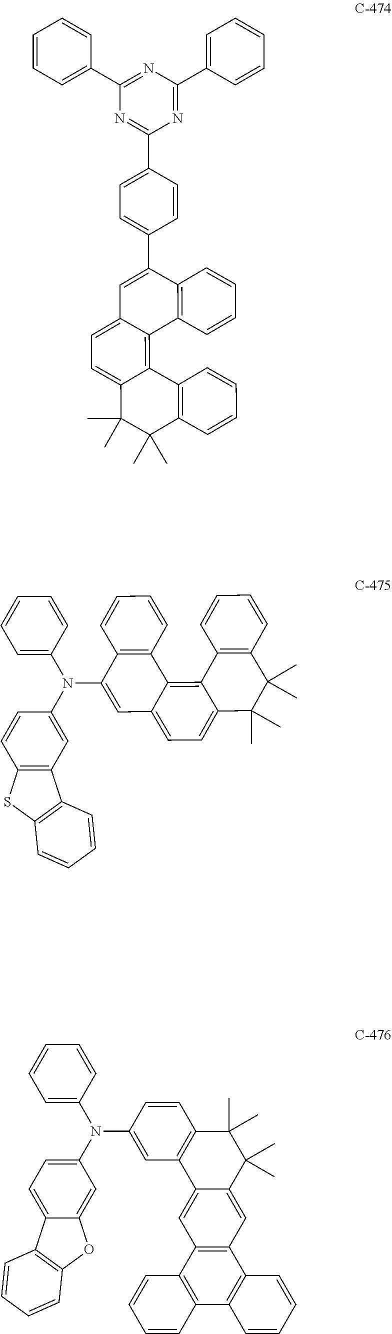 [SMILES: CC1(C)c2ccccc2-c2c(ccc3cc(-c4ccc(-c5nc(-c6ccccc6)nc(-c6ccccc6)n5)cc4)c4ccccc4c23)C1(C)C ; CC1(C)c2ccccc2-c2c(ccc3cc(N(c4ccccc4)c4ccc5sc6ccccc6c5c4)c4ccccc4c23)C1(C)C ; CC1(C)c2ccc(N(c3ccccc3)c3ccc4c(c3)oc3ccccc34)cc2-c2cc3c4ccccc4c4ccccc4c3cc2C1(C)C]

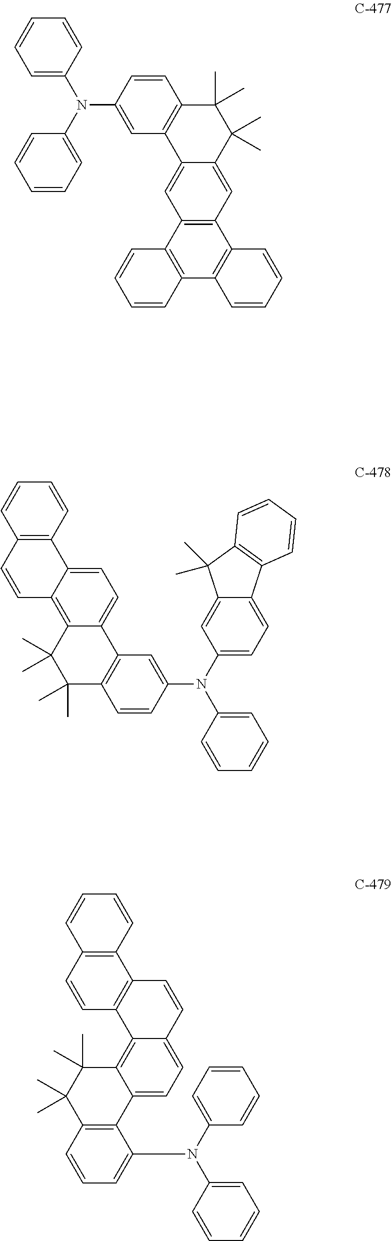 [SMILES: CC1(C)c2ccc(N(c3ccccc3)c3ccccc3)cc2-c2cc3c4ccccc4c4ccccc4c3cc2C1(C)C ; CC1(C)c2ccccc2-c2ccc(N(c3ccccc3)c3ccc4c(c3)-c3ccc5c(ccc6ccccc65)c3C(C)(C)C4(C)C)cc21 ; CC1(C)c2cccc(N(c3ccccc3)c3ccccc3)c2-c2ccc3ccc4c5ccccc5ccc4c3c2C1(C)C]

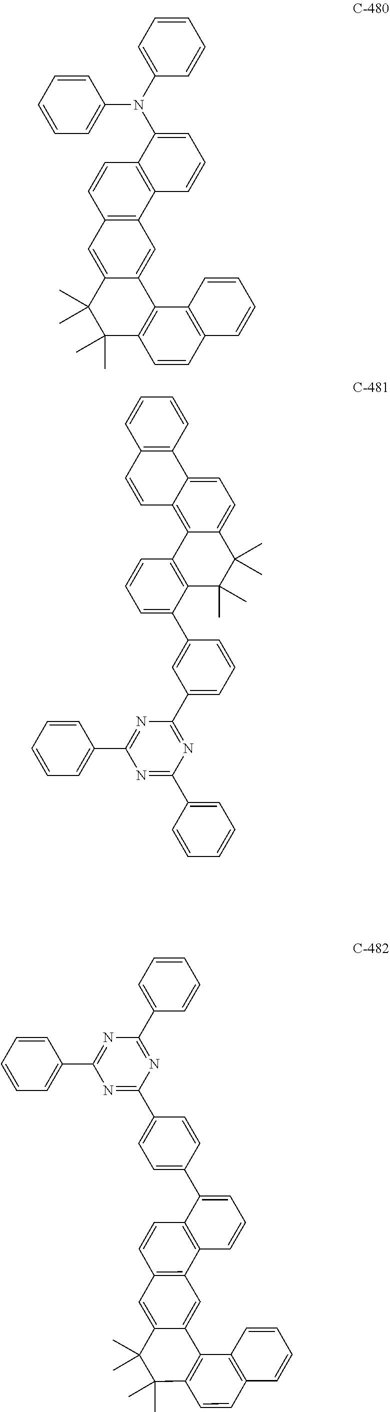

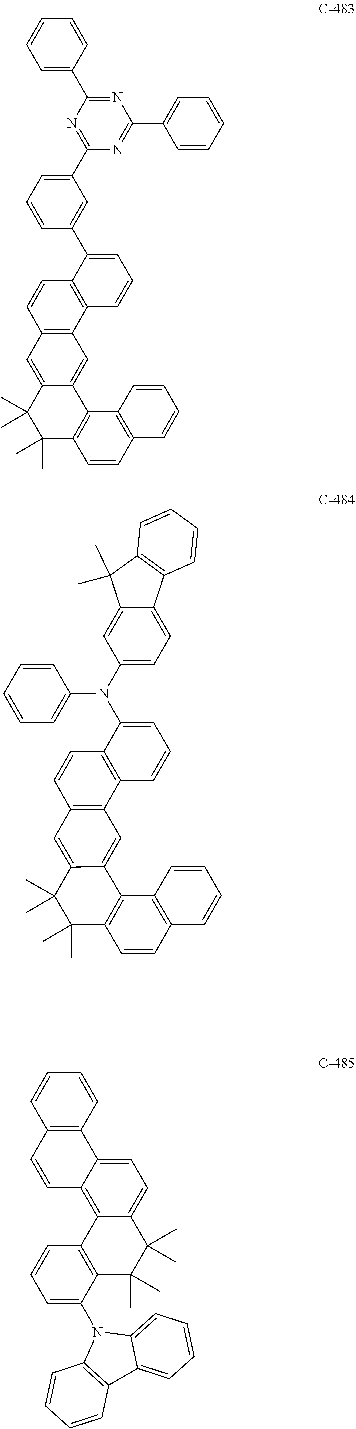 [SMILES: CC1(C)c2cc3ccc4c(-c5cccc(-c6nc(-c7ccccc7)nc(-c7ccccc7)n6)c5)cccc4c3cc2-c2c(ccc3ccccc23)C1(C)C ; CC1(C)c2ccccc2-c2ccc(N(c3ccccc3)c3cccc4c3ccc3cc5c(cc34)-c3c(ccc4ccccc34)C(C)(C)C5(C)C)cc21 ; CC1(C)c2ccc3c(ccc4ccccc43)c2-c2cccc(-n3c4ccccc4c4ccccc43)c2C1(C)C]

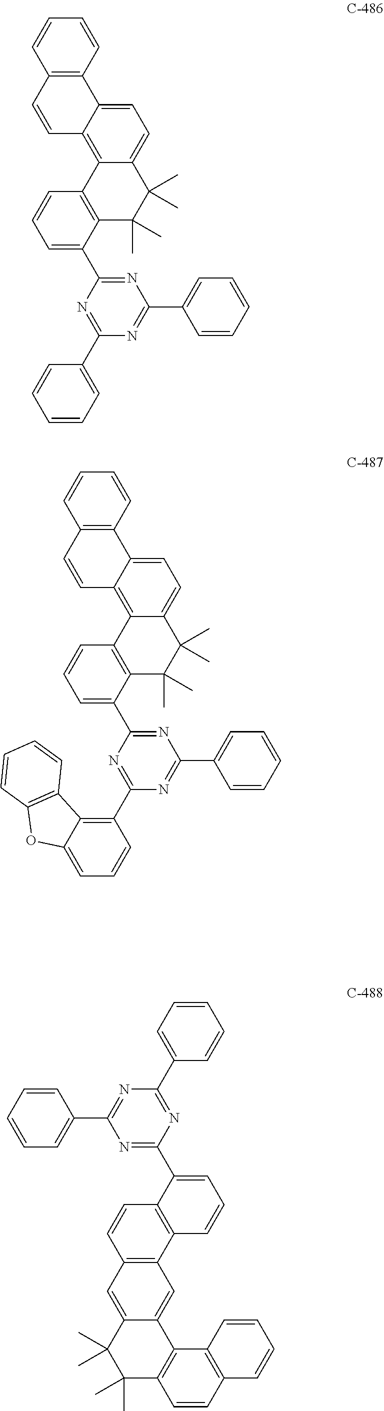 [SMILES: CC1(C)c2ccc3c(ccc4ccccc43)c2-c2cccc(-c3nc(-c4ccccc4)nc(-c4ccccc4)n3)c2C1(C)C ; CC1(C)c2ccc3c(ccc4ccccc43)c2-c2cccc(-c3nc(-c4ccccc4)nc(-c4cccc5oc6ccccc6c45)n3)c2C1(C)C ; CC1(C)c2cc3ccc4c(-c5nc(-c6ccccc6)nc(-c6ccccc6)n5)cccc4c3cc2-c2c(ccc3ccccc23)C1(C)C]

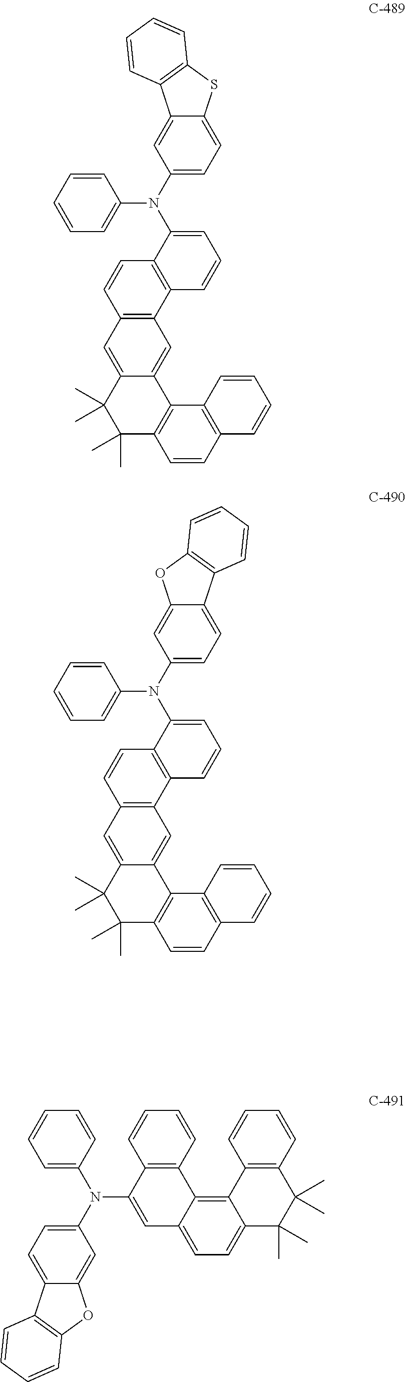 [SMILES: CC1(C)c2cc3ccc4c(N(c5ccccc5)c5ccc6sc7ccccc7c6c5)cccc4c3cc2-c2c(ccc3ccccc23)C1(C)C ; CC1(C)c2cc3ccc4c(N(c5ccccc5)c5ccc6c(c5)oc5ccccc56)cccc4c3cc2-c2c(ccc3ccccc23)C1(C)C ; CC1(C)c2ccccc2-c2c(ccc3cc(N(c4ccccc4)c4ccc5c(c4)oc4ccccc45)c4ccccc4c23)C1(C)C]

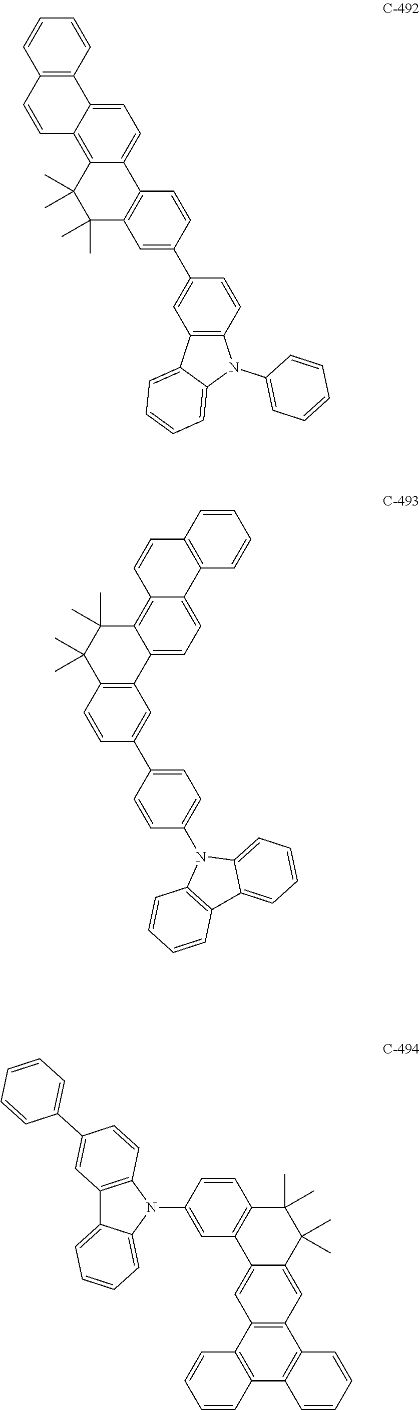 [SMILES: CC1(C)c2cc(-c3ccc4c(c3)c3ccccc3n4-c3ccccc3)ccc2-c2ccc3c(ccc4ccccc43)c2C1(C)C ; CC1(C)c2ccc(-c3ccc(-n4c5ccccc5c5ccccc54)cc3)cc2-c2ccc3c(ccc4ccccc43)c2C1(C)C ; CC1(C)c2ccc(-n3c4ccccc4c4cc(-c5ccccc5)ccc43)cc2-c2cc3c4ccccc4c4ccccc4c3cc2C1(C)C]

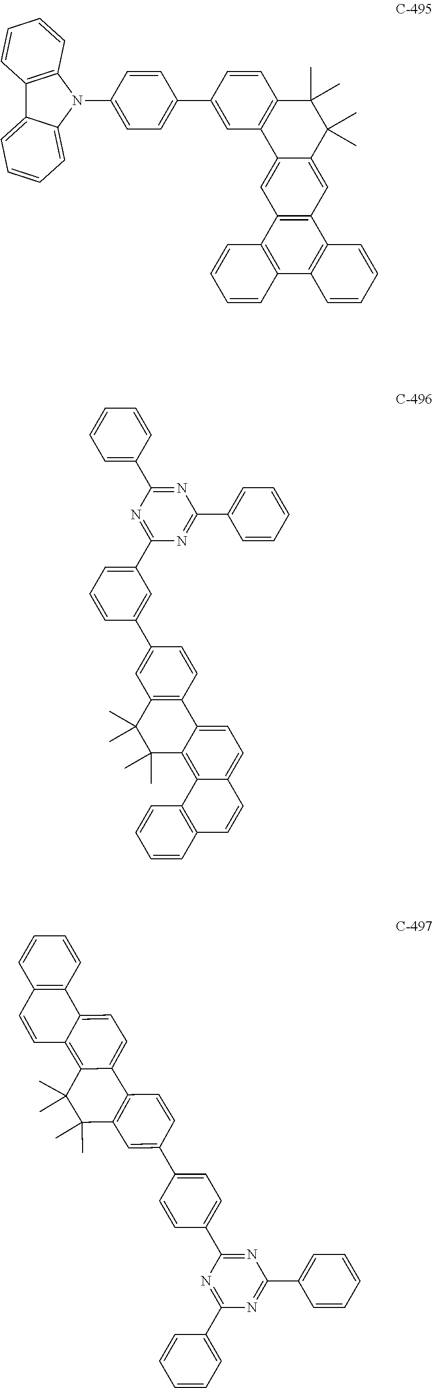 [SMILES: CC1(C)c2ccc(-c3ccc(-n4c5ccccc5c5ccccc54)cc3)cc2-c2cc3c4ccccc4c4ccccc4c3cc2C1(C)C ; CC1(C)c2cc(-c3cccc(-c4nc(-c5ccccc5)nc(-c5ccccc5)n4)c3)ccc2-c2ccc3ccc4ccccc4c3c2C1(C)C ; CC1(C)c2cc(-c3ccc(-c4nc(-c5ccccc5)nc(-c5ccccc5)n4)cc3)ccc2-c2ccc3c(ccc4ccccc43)c2C1(C)C]

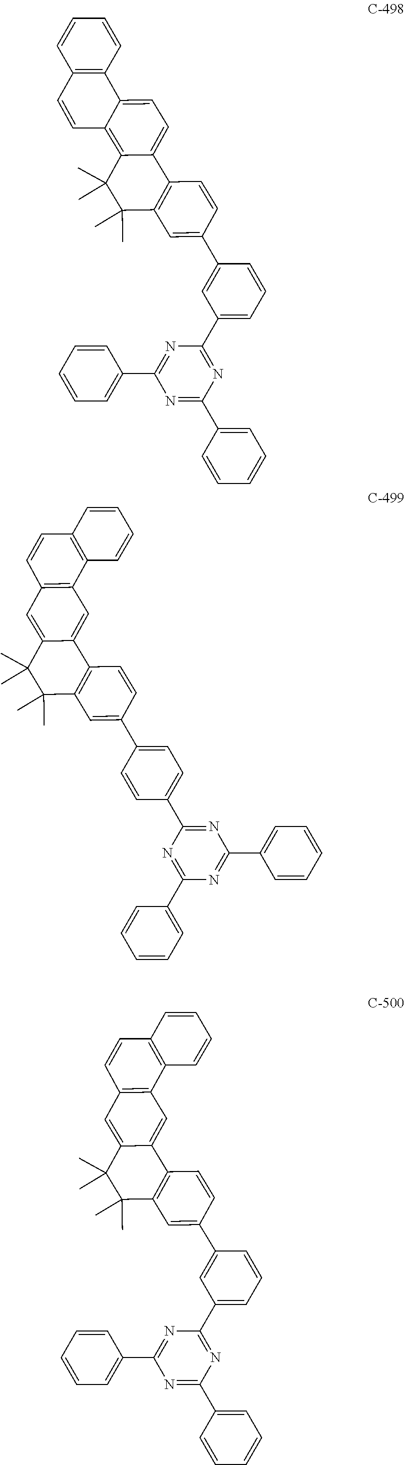 [SMILES: CC1(C)c2cc(-c3cccc(-c4nc(-c5ccccc5)nc(-c5ccccc5)n4)c3)ccc2-c2ccc3c(ccc4ccccc43)c2C1(C)C ; CC1(C)c2cc(-c3ccc(-c4nc(-c5ccccc5)nc(-c5ccccc5)n4)cc3)ccc2-c2cc3c(ccc4ccccc43)cc2C1(C)C ; CC1(C)c2cc(-c3cccc(-c4nc(-c5ccccc5)nc(-c5ccccc5)n4)c3)ccc2-c2cc3c(ccc4ccccc43)cc2C1(C)C]

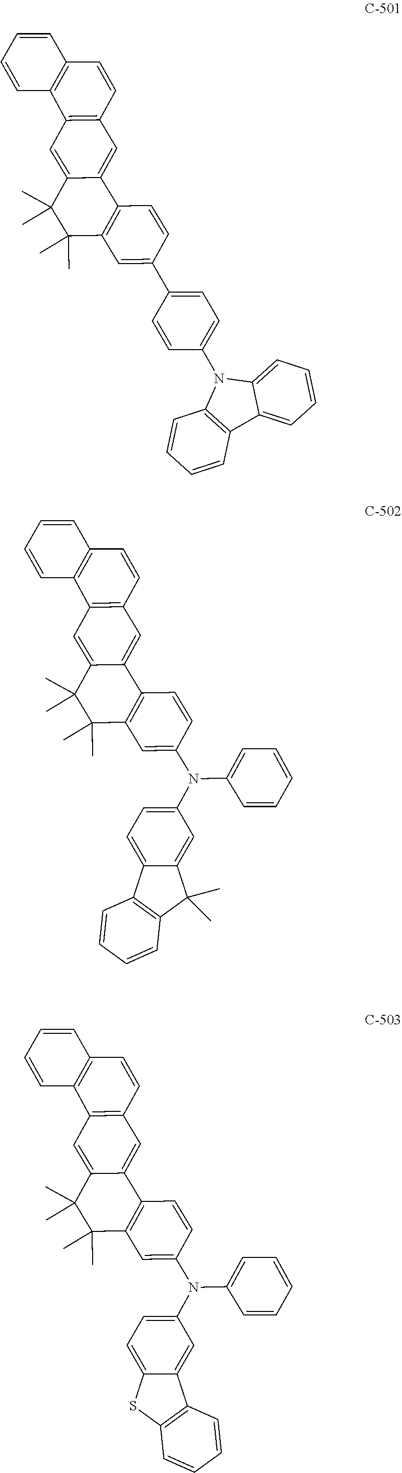 [SMILES: CC1(C)c2cc(-c3ccc(-n4c5ccccc5c5ccccc54)cc3)ccc2-c2cc3ccc4ccccc4c3cc2C1(C)C ; CC1(C)c2ccccc2-c2ccc(N(c3ccccc3)c3ccc4c(c3)C(C)(C)C(C)(C)c3cc5c(ccc6ccccc65)cc3-4)cc21 ; CC1(C)c2cc(N(c3ccccc3)c3ccc4sc5ccccc5c4c3)ccc2-c2cc3ccc4ccccc4c3cc2C1(C)C]

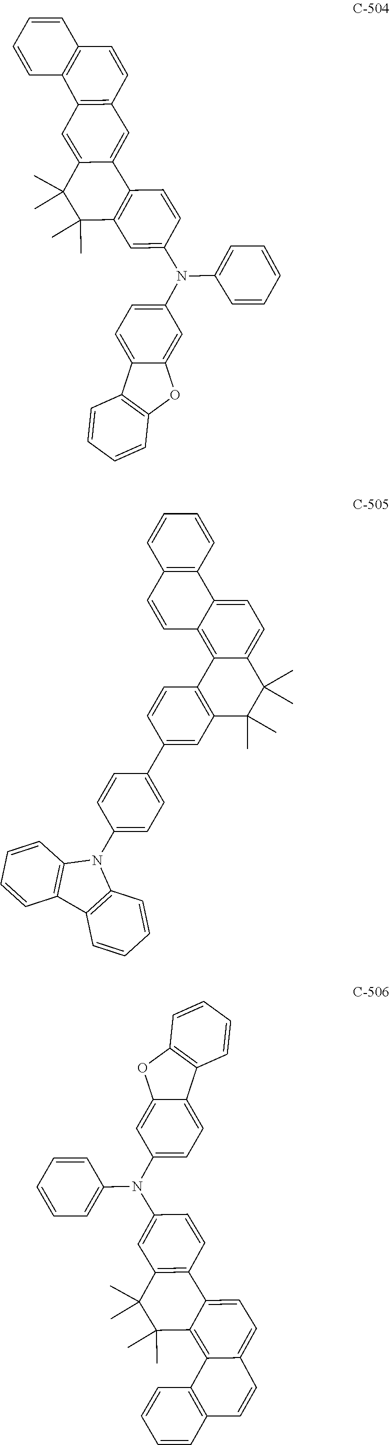 [SMILES: CC1(C)c2cc(N(c3ccccc3)c3ccc4c(c3)oc3ccccc34)ccc2-c2cc3ccc4ccccc4c3cc2C1(C)C ; CC1(C)c2cc(-c3ccc(-n4c5ccccc5c5ccccc54)cc3)ccc2-c2c(ccc3c2ccc2ccccc23)C1(C)C ; CC1(C)c2cc(N(c3ccccc3)c3ccc4c(c3)oc3ccccc34)ccc2-c2ccc3ccc4ccccc4c3c2C1(C)C]

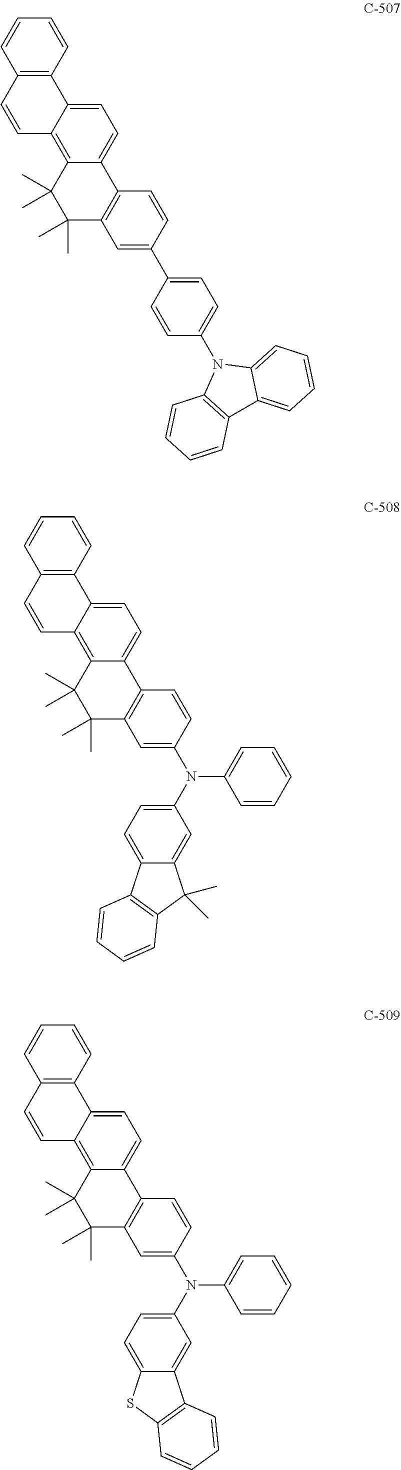 [SMILES: CC1(C)c2cc(-c3ccc(-n4c5ccccc5c5ccccc54)cc3)ccc2-c2ccc3c(ccc4ccccc43)c2C1(C)C ; CC1(C)c2ccccc2-c2ccc(N(c3ccccc3)c3ccc4c(c3)C(C)(C)C(C)(C)c3c-4ccc4c3ccc3ccccc34)cc21 ; CC1(C)c2cc(N(c3ccccc3)c3ccc4sc5ccccc5c4c3)ccc2-c2ccc3c(ccc4ccccc43)c2C1(C)C]

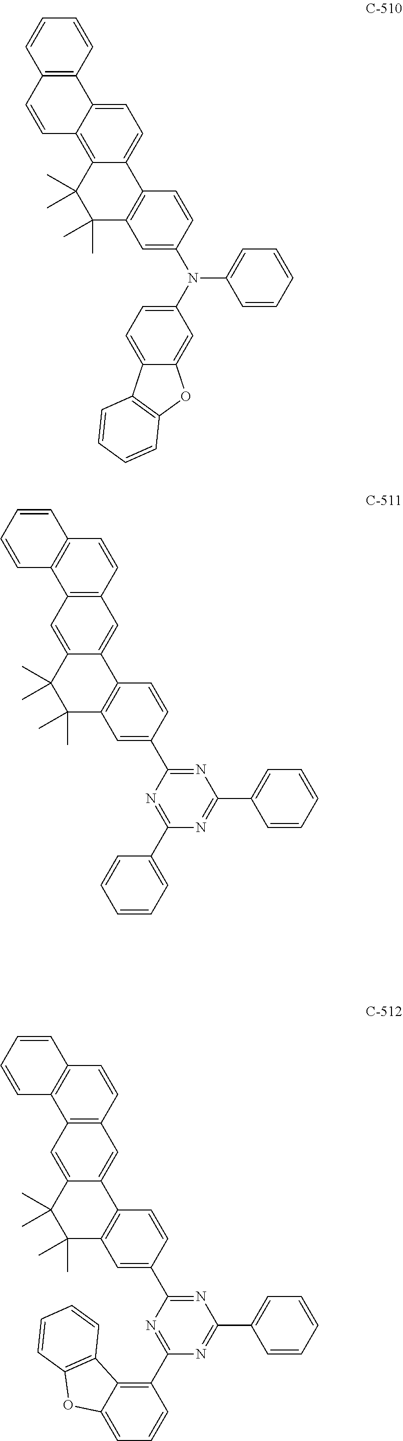 [SMILES: CC1(C)c2cc(N(c3ccccc3)c3ccc4c(c3)oc3ccccc34)ccc2-c2ccc3c(ccc4ccccc43)c2C1(C)C ; CC1(C)c2cc(-c3nc(-c4ccccc4)nc(-c4ccccc4)n3)ccc2-c2cc3ccc4ccccc4c3cc2C1(C)C ; CC1(C)c2cc(-c3nc(-c4ccccc4)nc(-c4cccc5oc6ccccc6c45)n3)ccc2-c2cc3ccc4ccccc4c3cc2C1(C)C]

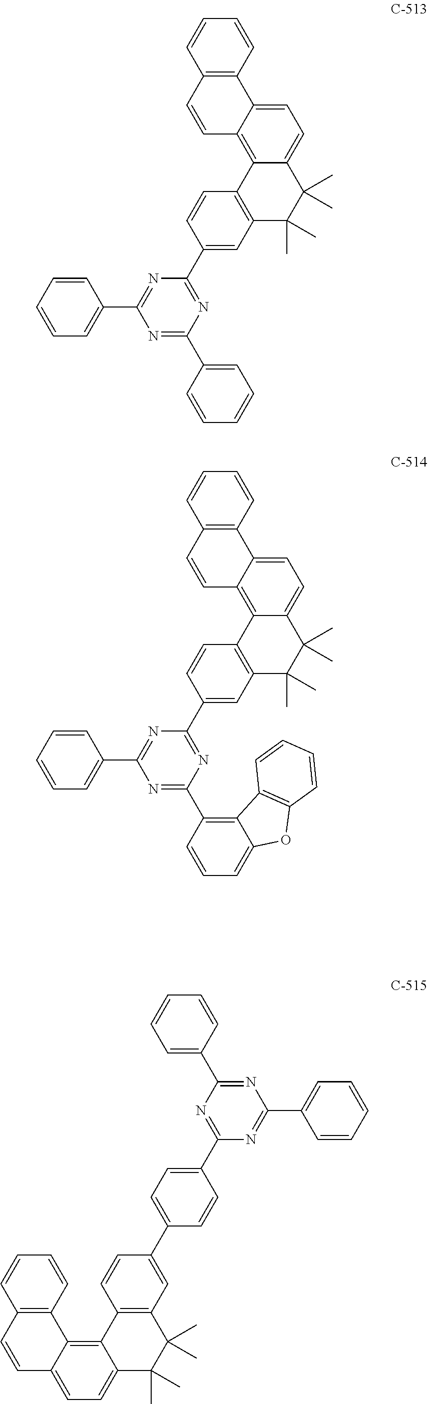 [SMILES: CC1(C)c2cc(-c3nc(-c4ccccc4)nc(-c4ccccc4)n3)ccc2-c2c(ccc3c2ccc2ccccc23)C1(C)C ; CC1(C)c2cc(-c3nc(-c4ccccc4)nc(-c4cccc5oc6ccccc6c45)n3)ccc2-c2c(ccc3c2ccc2ccccc23)C1(C)C ; CC1(C)c2cc(-c3ccc(-c4nc(-c5ccccc5)nc(-c5ccccc5)n4)cc3)ccc2-c2c(ccc3ccc4ccccc4c23)C1(C)C]

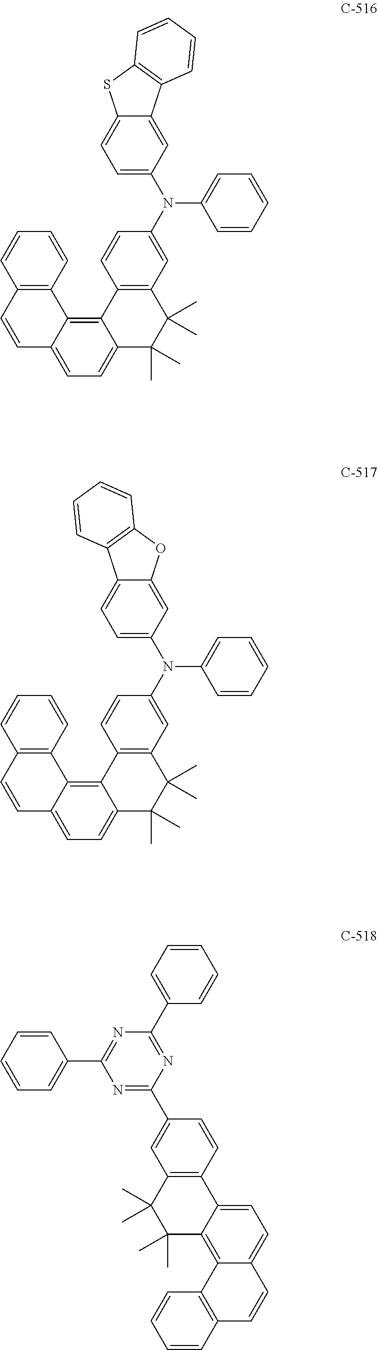 [SMILES: CC1(C)c2cc(N(c3ccccc3)c3ccc4sc5ccccc5c4c3)ccc2-c2c(ccc3ccc4ccccc4c23)C1(C)C ; CC1(C)c2cc(N(c3ccccc3)c3ccc4c(c3)oc3ccccc34)ccc2-c2c(ccc3ccc4ccccc4c23)C1(C)C ; CC1(C)c2cc(-c3nc(-c4ccccc4)nc(-c4ccccc4)n3)ccc2-c2ccc3ccc4ccccc4c3c2C1(C)C]

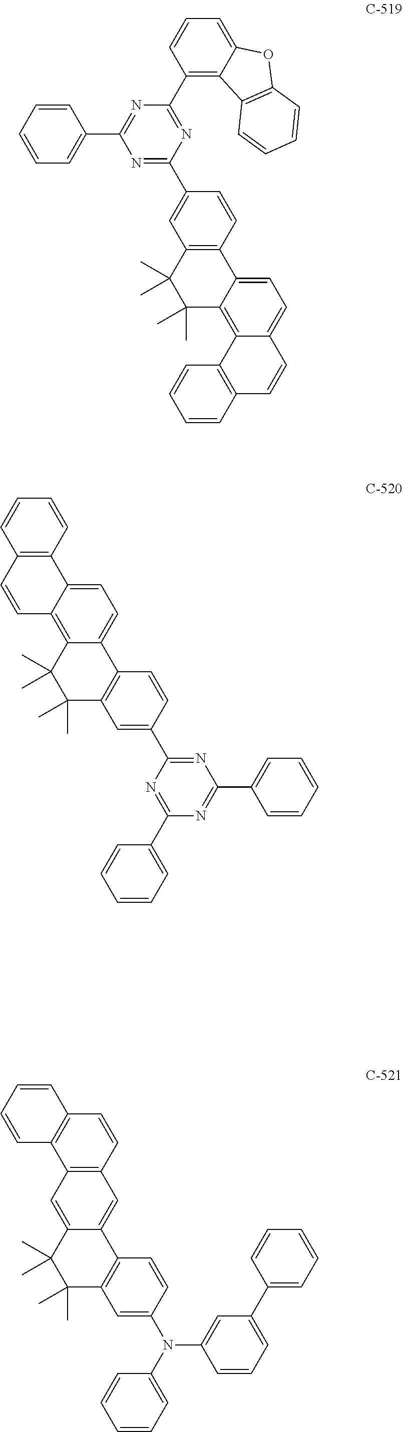 [SMILES: CC1(C)c2cc(-c3nc(-c4ccccc4)nc(-c4cccc5oc6ccccc6c45)n3)ccc2-c2ccc3ccc4ccccc4c3c2C1(C)C ; CC1(C)c2cc(-c3nc(-c4ccccc4)nc(-c4ccccc4)n3)ccc2-c2ccc3c(ccc4ccccc43)c2C1(C)C ; CC1(C)c2cc(N(c3ccccc3)c3cccc(-c4ccccc4)c3)ccc2-c2cc3ccc4ccccc4c3cc2C1(C)C]

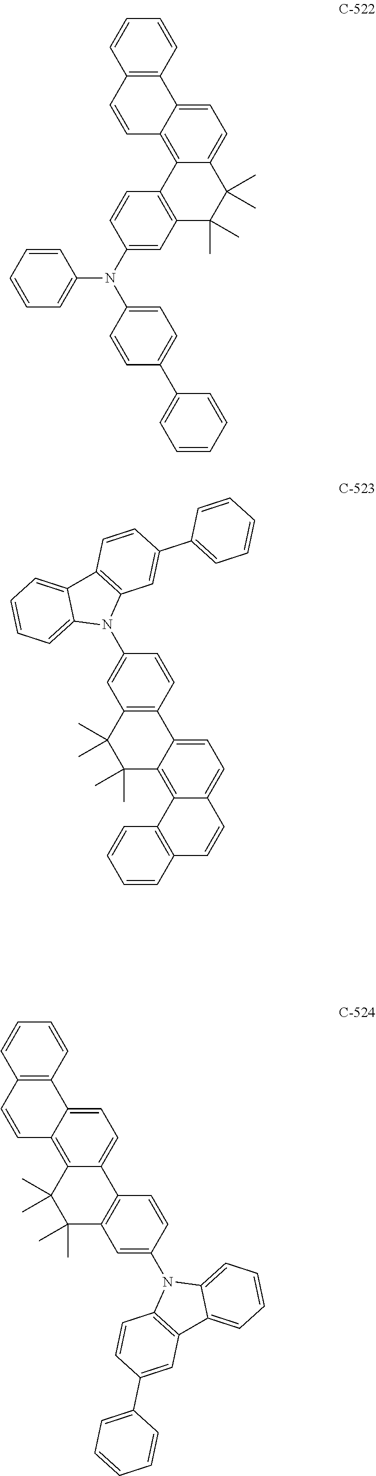 [SMILES: CC1(C)c2cc(N(c3ccccc3)c3ccc(-c4ccccc4)cc3)ccc2-c2c(ccc3c2ccc2ccccc23)C1(C)C ; CC1(C)c2cc(-n3c4ccccc4c4ccc(-c5ccccc5)cc43)ccc2-c2ccc3ccc4ccccc4c3c2C1(C)C ; CC1(C)c2cc(-n3c4ccccc4c4cc(-c5ccccc5)ccc43)ccc2-c2ccc3c(ccc4ccccc43)c2C1(C)C]

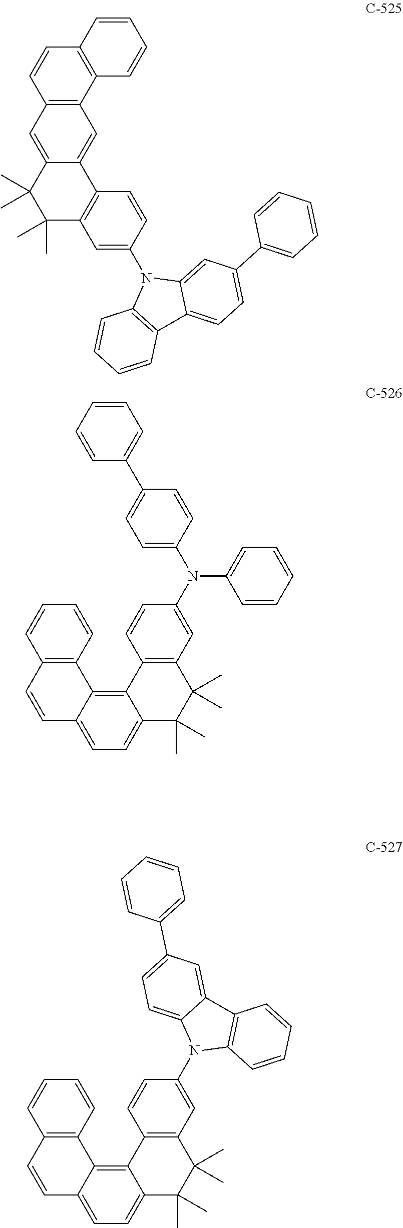

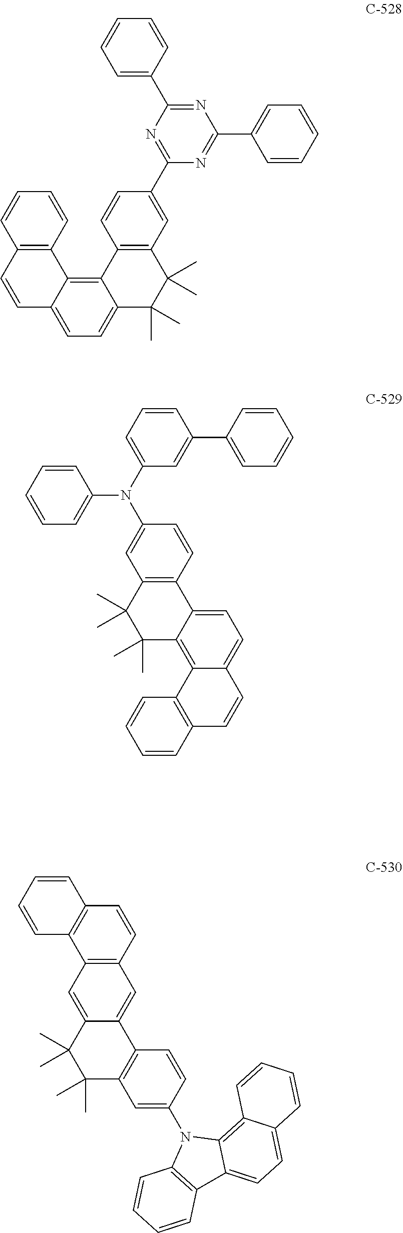 [SMILES: CC1(C)c2cc(-c3nc(-c4ccccc4)nc(-c4ccccc4)n3)ccc2-c2c(ccc3ccc4ccccc4c23)C1(C)C ; CC1(C)c2cc(N(c3ccccc3)c3cccc(-c4ccccc4)c3)ccc2-c2ccc3ccc4ccccc4c3c2C1(C)C ; CC1(C)c2cc(-n3c4ccccc4c4ccc5ccccc5c43)ccc2-c2cc3ccc4ccccc4c3cc2C1(C)C]

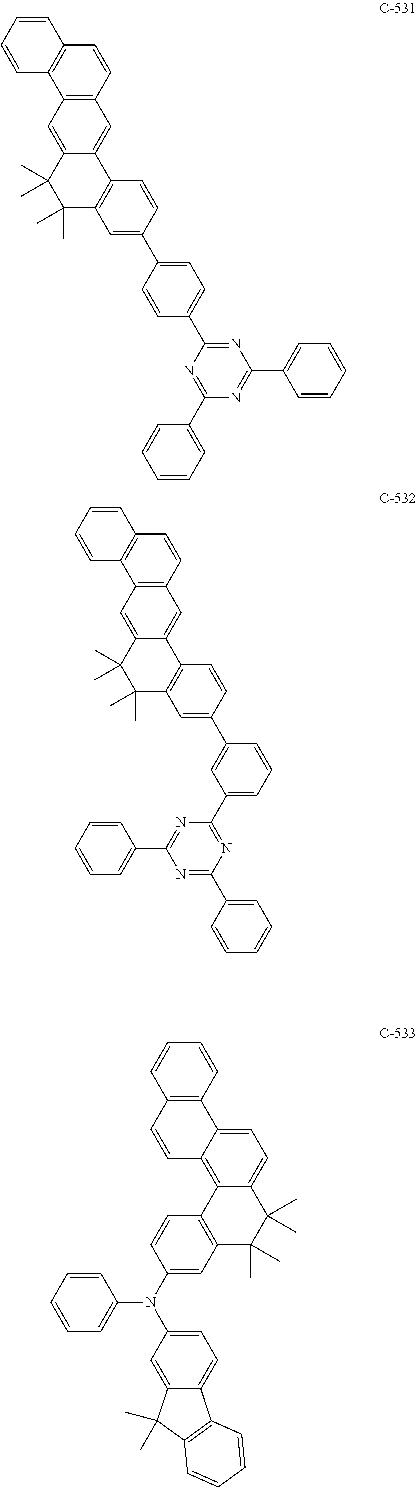 [SMILES: CC1(C)c2cc(-c3ccc(-c4nc(-c5ccccc5)nc(-c5ccccc5)n4)cc3)ccc2-c2cc3ccc4ccccc4c3cc2C1(C)C ; CC1(C)c2cc(-c3cccc(-c4nc(-c5ccccc5)nc(-c5ccccc5)n4)c3)ccc2-c2cc3ccc4ccccc4c3cc2C1(C)C ; CC1(C)c2ccccc2-c2ccc(N(c3ccccc3)c3ccc4c(c3)C(C)(C)C(C)(C)c3ccc5c(ccc6ccccc65)c3-4)cc21]

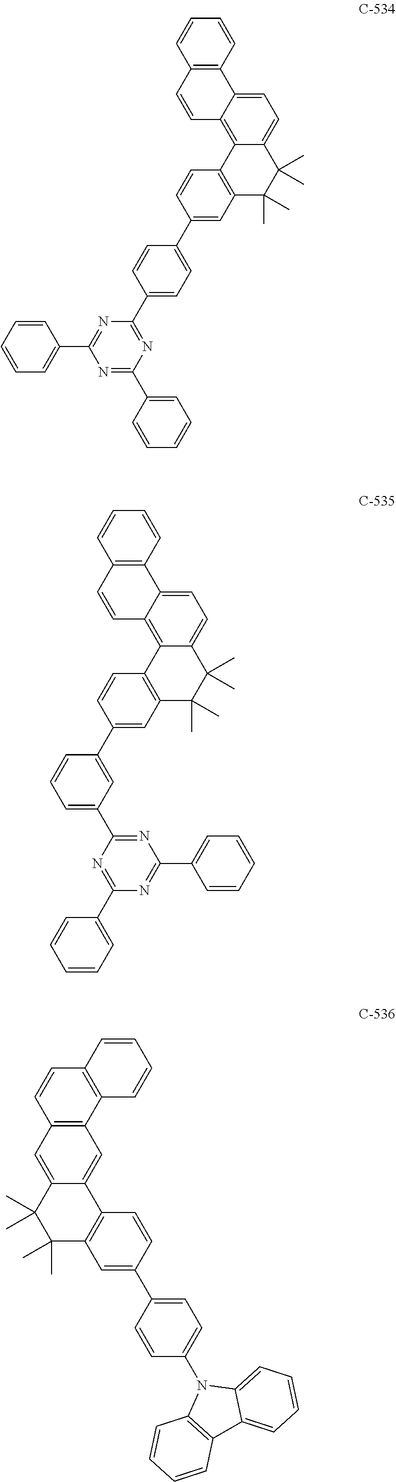

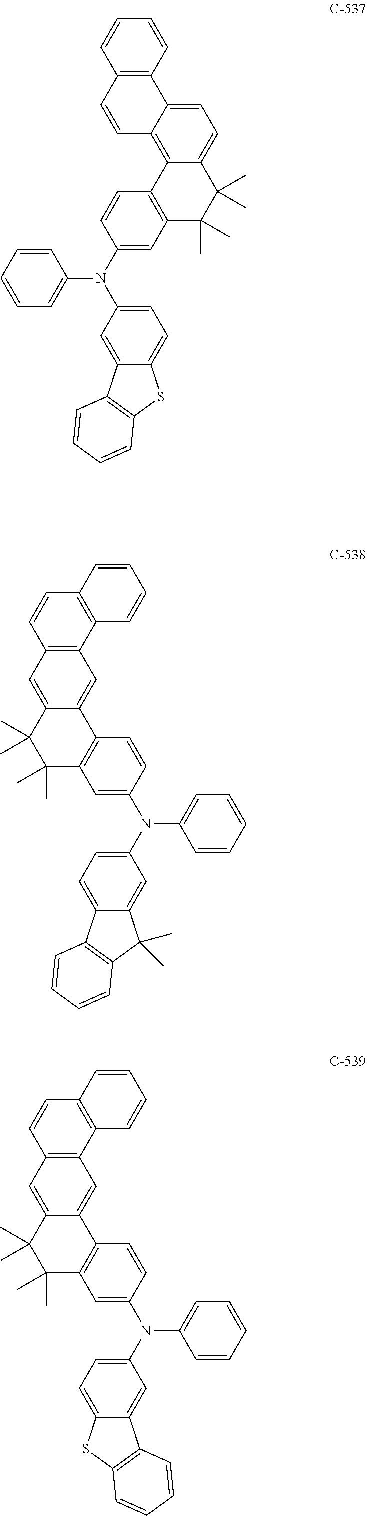

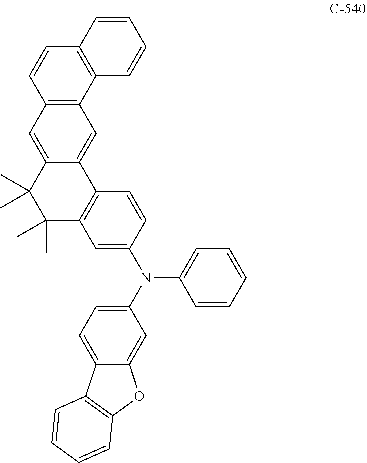 [SMILES: CC1(C)c2cc(N(c3ccccc3)c3ccc4c(c3)oc3ccccc34)ccc2-c2cc3c(ccc4ccccc43)cc2C1(C)C]

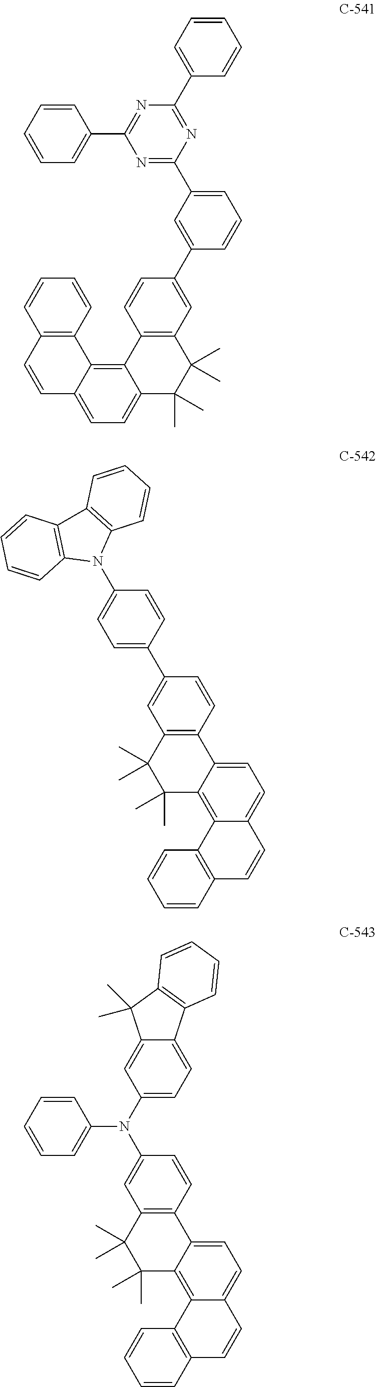

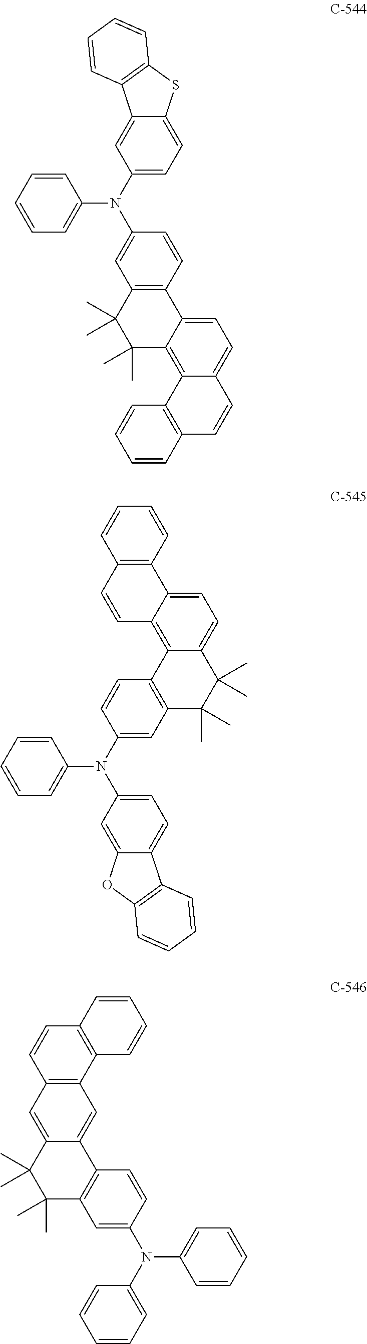

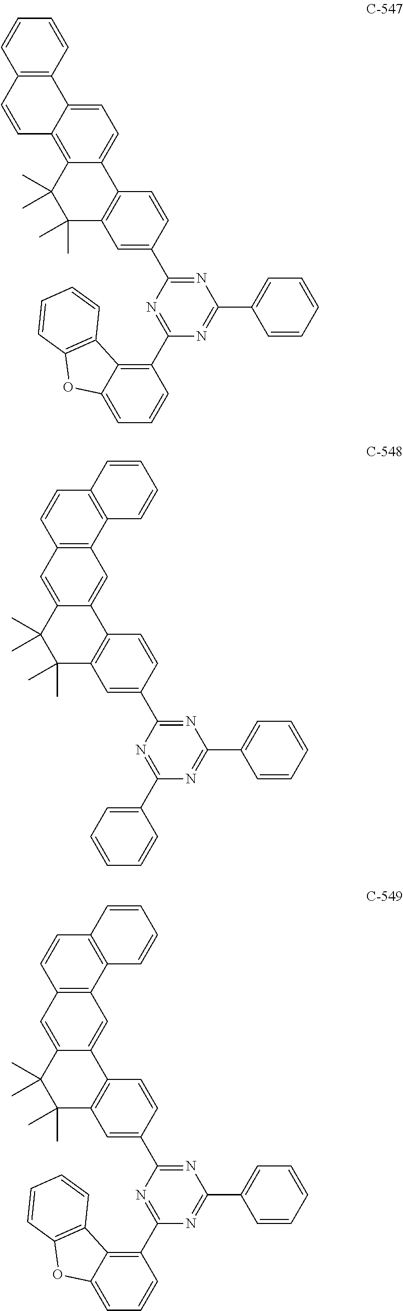

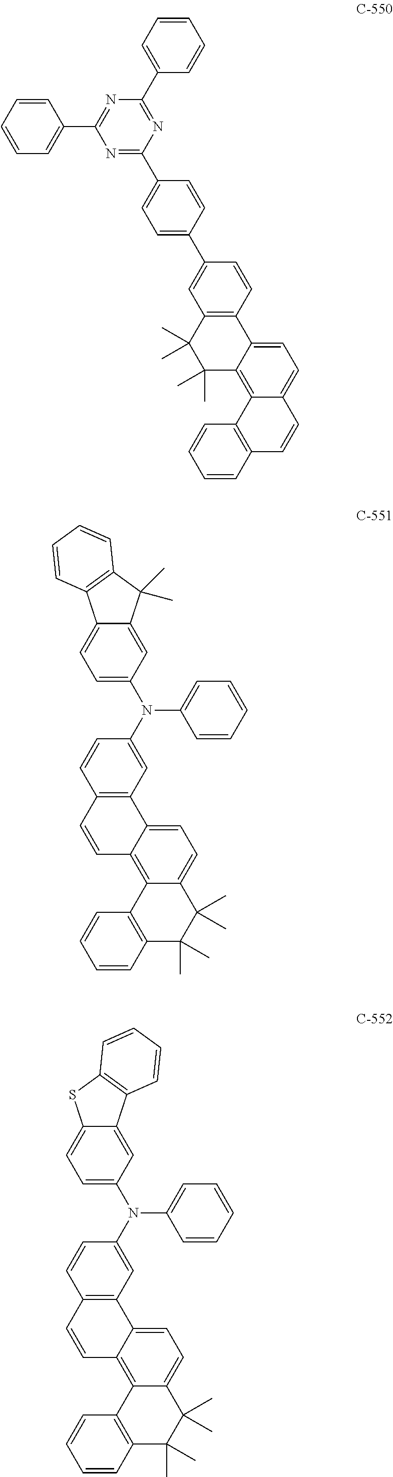 [SMILES: CC1(C)c2cc(-c3ccc(-c4nc(-c5ccccc5)nc(-c5ccccc5)n4)cc3)ccc2-c2ccc3ccc4ccccc4c3c2C1(C)C ; CC1(C)c2ccccc2-c2ccc(N(c3ccccc3)c3ccc4ccc5c6c(ccc5c4c3)C(C)(C)C(C)(C)c3ccccc3-6)cc21 ; CC1(C)c2ccccc2-c2c(ccc3c2ccc2ccc(N(c4ccccc4)c4ccc5sc6ccccc6c5c4)cc23)C1(C)C]

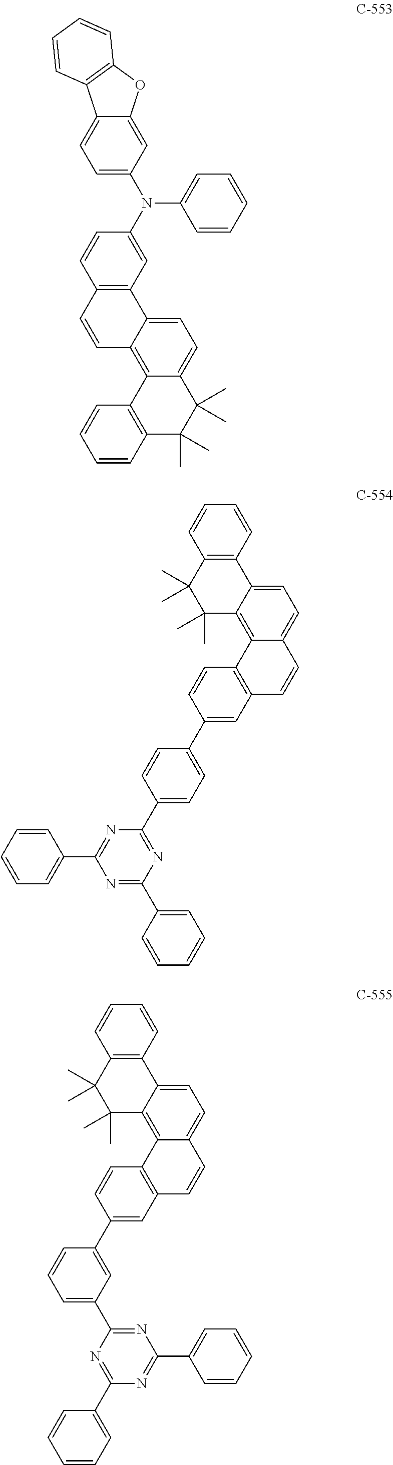

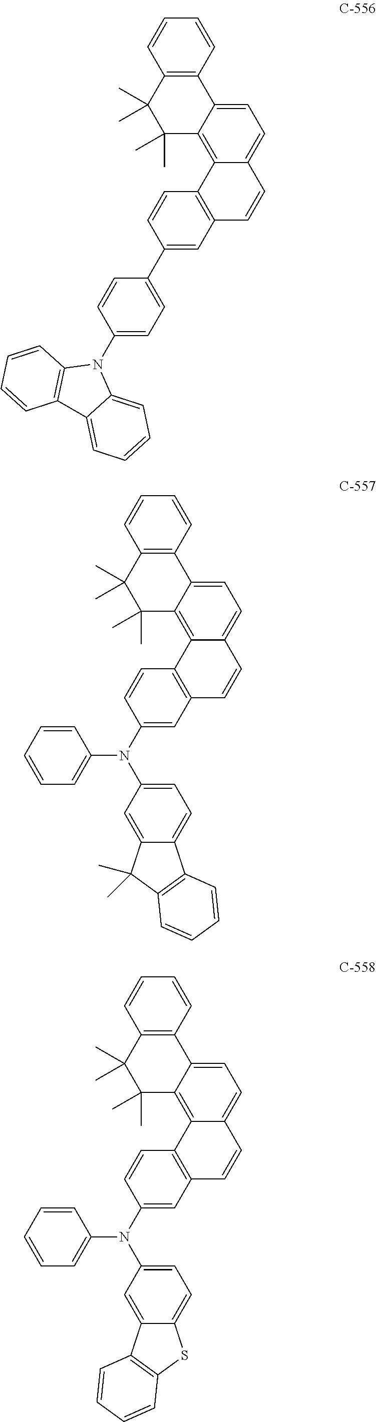

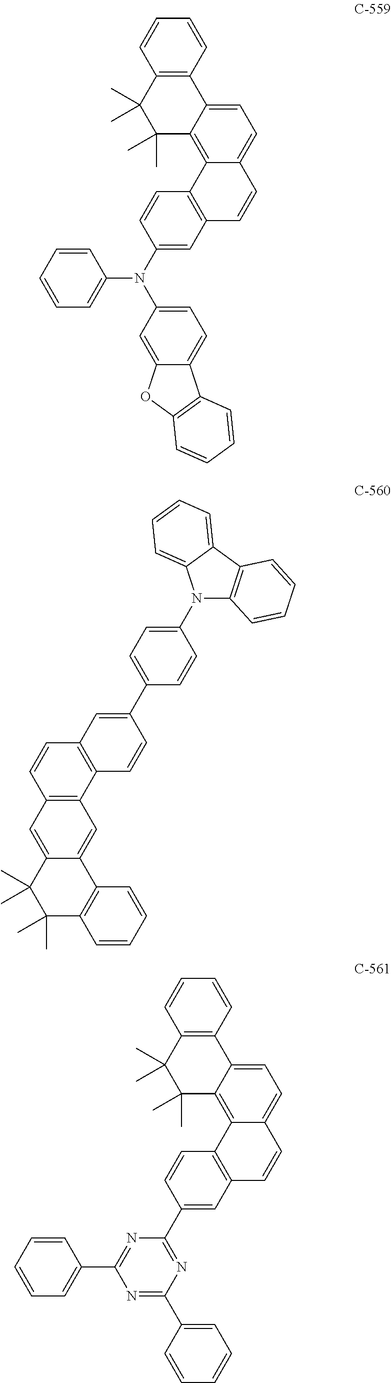

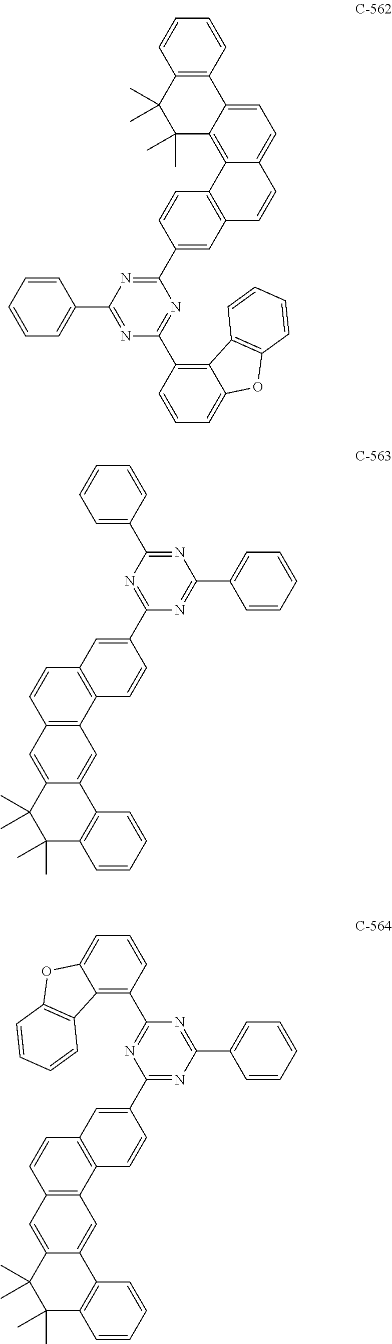 [SMILES: CC1(C)c2ccccc2-c2ccc3ccc4cc(-c5nc(-c6ccccc6)nc(-c6cccc7oc8ccccc8c67)n5)ccc4c3c2C1(C)C ; CC1(C)c2ccccc2-c2cc3c(ccc4cc(-c5nc(-c6ccccc6)nc(-c6ccccc6)n5)ccc43)cc2C1(C)C ; CC1(C)c2ccccc2-c2cc3c(ccc4cc(-c5nc(-c6ccccc6)nc(-c6cccc7oc8ccccc8c67)n5)ccc43)cc2C1(C)C]

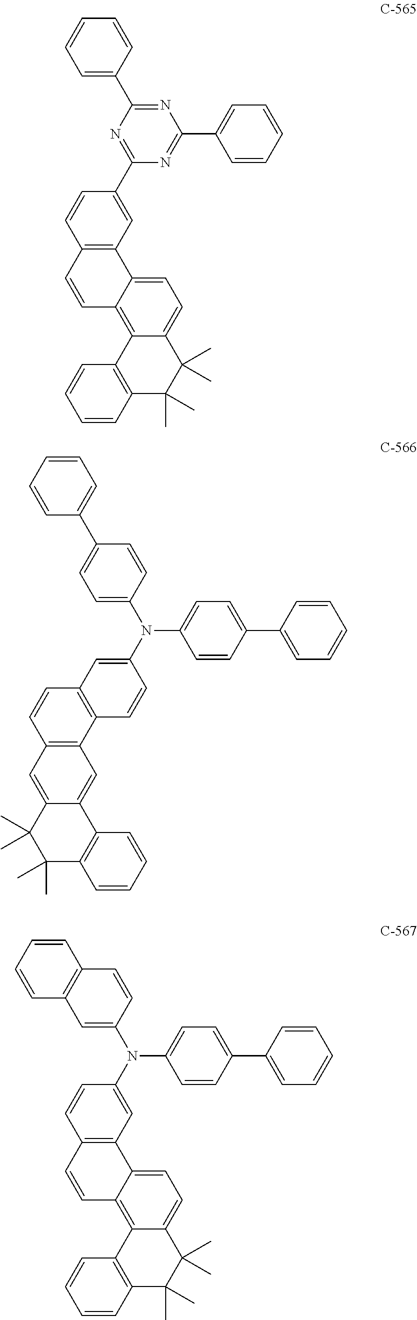

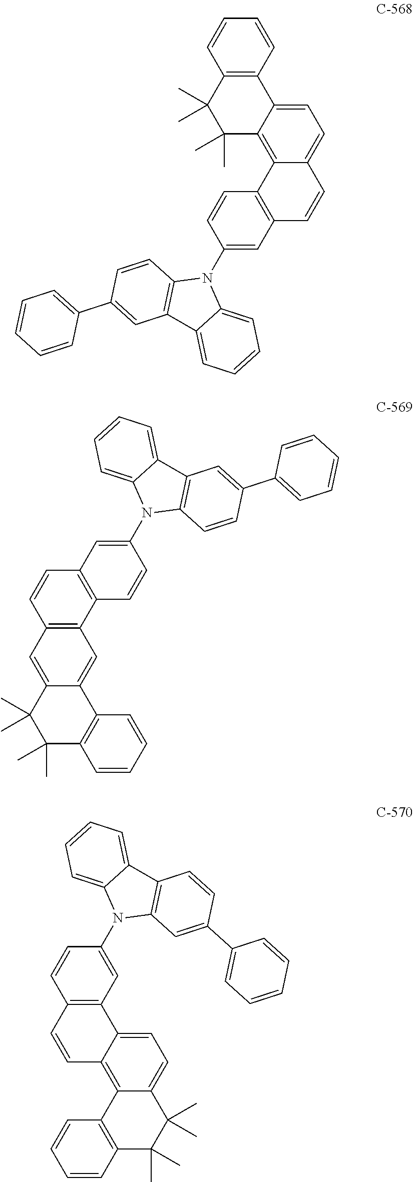 [SMILES: CC1(C)c2ccccc2-c2ccc3ccc4cc(-n5c6ccccc6c6cc(-c7ccccc7)ccc65)ccc4c3c2C1(C)C ; CC1(C)c2ccccc2-c2cc3c(ccc4cc(-n5c6ccccc6c6cc(-c7ccccc7)ccc65)ccc43)cc2C1(C)C ; CC1(C)c2ccccc2-c2c(ccc3c2ccc2ccc(-n4c5ccccc5c5ccc(-c6ccccc6)cc54)cc23)C1(C)C]

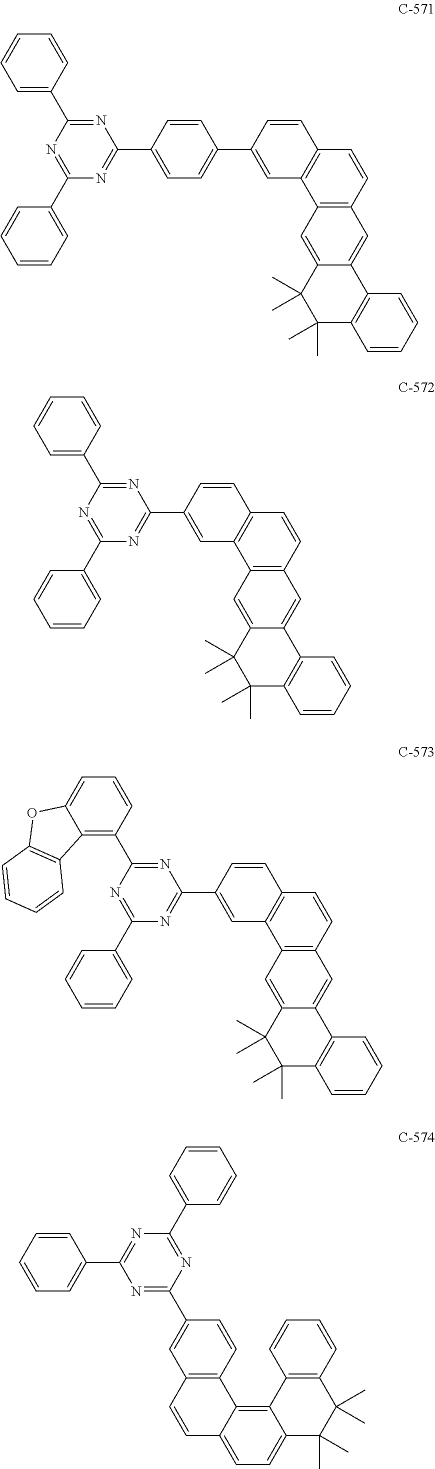 [SMILES: CC1(C)c2ccccc2-c2cc3ccc4ccc(-c5ccc(-c6nc(-c7ccccc7)nc(-c7ccccc7)n6)cc5)cc4c3cc2C1(C)C ; CC1(C)c2ccccc2-c2cc3ccc4ccc(-c5nc(-c6ccccc6)nc(-c6ccccc6)n5)cc4c3cc2C1(C)C ; CC1(C)c2ccccc2-c2cc3ccc4ccc(-c5nc(-c6ccccc6)nc(-c6cccc7oc8ccccc8c67)n5)cc4c3cc2C1(C)C ; CC1(C)c2ccccc2-c2c(ccc3ccc4cc(-c5nc(-c6ccccc6)nc(-c6ccccc6)n5)ccc4c23)C1(C)C]

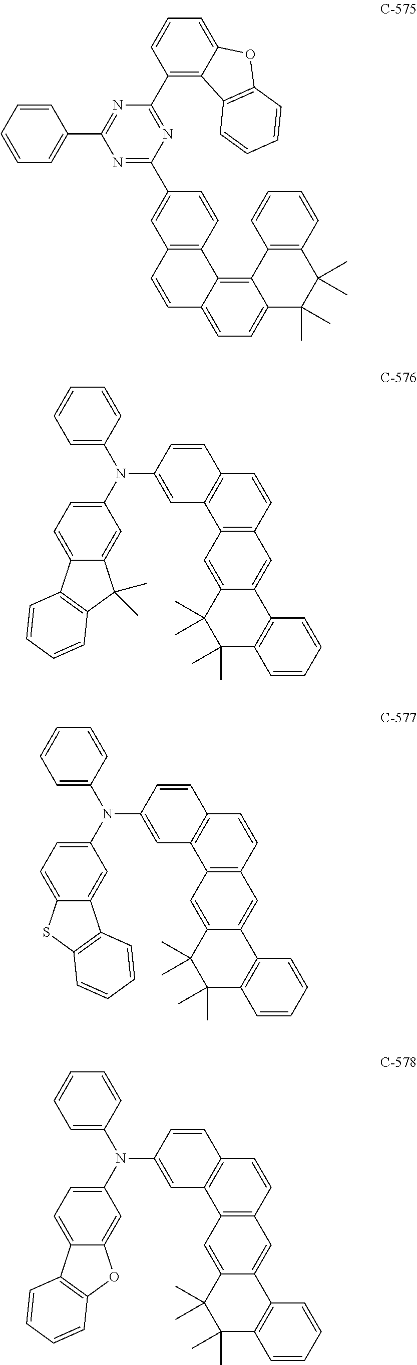 [SMILES: CC1(C)c2ccccc2-c2c(ccc3ccc4cc(-c5nc(-c6ccccc6)nc(-c6cccc7oc8ccccc8c67)n5)ccc4c23)C1(C)C ; CC1(C)c2ccccc2-c2ccc(N(c3ccccc3)c3ccc4ccc5cc6c(cc5c4c3)C(C)(C)C(C)(C)c3ccccc3-6)cc21 ; CC1(C)c2ccccc2-c2cc3ccc4ccc(N(c5ccccc5)c5ccc6sc7ccccc7c6c5)cc4c3cc2C1(C)C ; CC1(C)c2ccccc2-c2cc3ccc4ccc(N(c5ccccc5)c5ccc6c(c5)oc5ccccc56)cc4c3cc2C1(C)C]

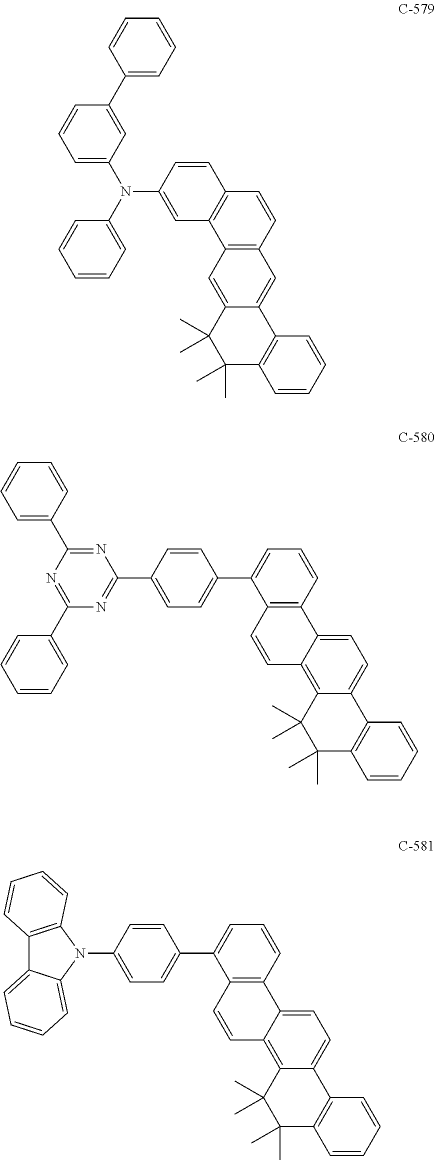 [SMILES: CC1(C)c2ccccc2-c2cc3ccc4ccc(N(c5ccccc5)c5cccc(-c6ccccc6)c5)cc4c3cc2C1(C)C ; CC1(C)c2ccccc2-c2ccc3c(ccc4c(-c5ccc(-c6nc(-c7ccccc7)nc(-c7ccccc7)n6)cc5)cccc43)c2C1(C)C ; CC1(C)c2ccccc2-c2ccc3c(ccc4c(-c5ccc(-n6c7ccccc7c7ccccc76)cc5)cccc43)c2C1(C)C]

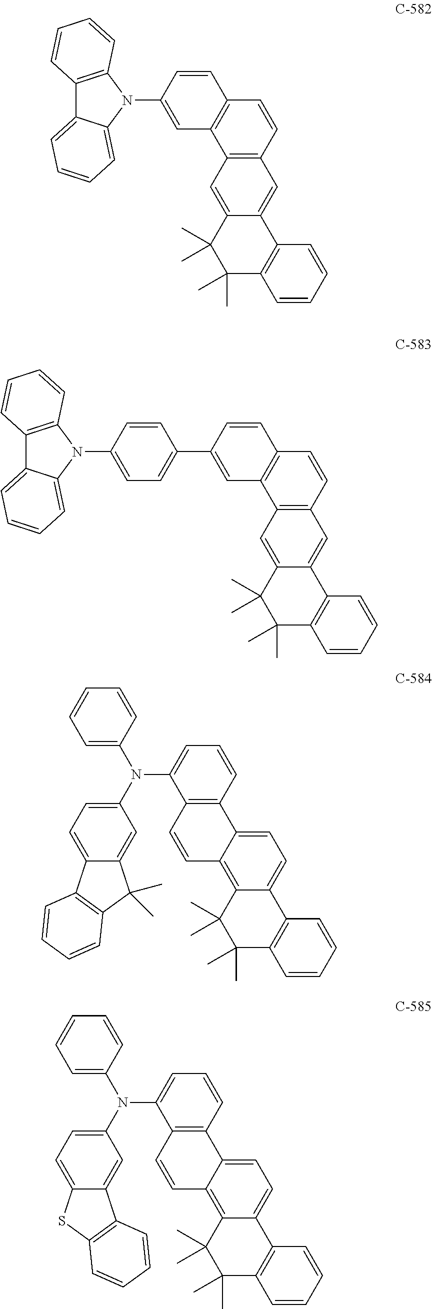 [SMILES: CC1(C)c2ccccc2-c2cc3ccc4ccc(-n5c6ccccc6c6ccccc65)cc4c3cc2C1(C)C ; CC1(C)c2ccccc2-c2cc3ccc4ccc(-c5ccc(-n6c7ccccc7c7ccccc76)cc5)cc4c3cc2C1(C)C ; CC1(C)c2ccccc2-c2ccc(N(c3ccccc3)c3cccc4c3ccc3c5c(ccc34)-c3ccccc3C(C)(C)C5(C)C)cc21 ; CC1(C)c2ccccc2-c2ccc3c(ccc4c(N(c5ccccc5)c5ccc6sc7ccccc7c6c5)cccc43)c2C1(C)C]

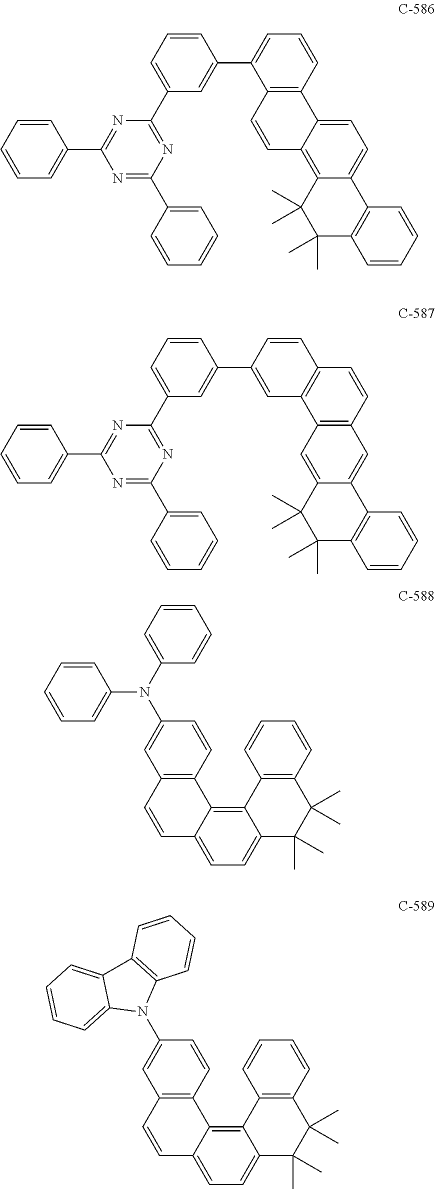

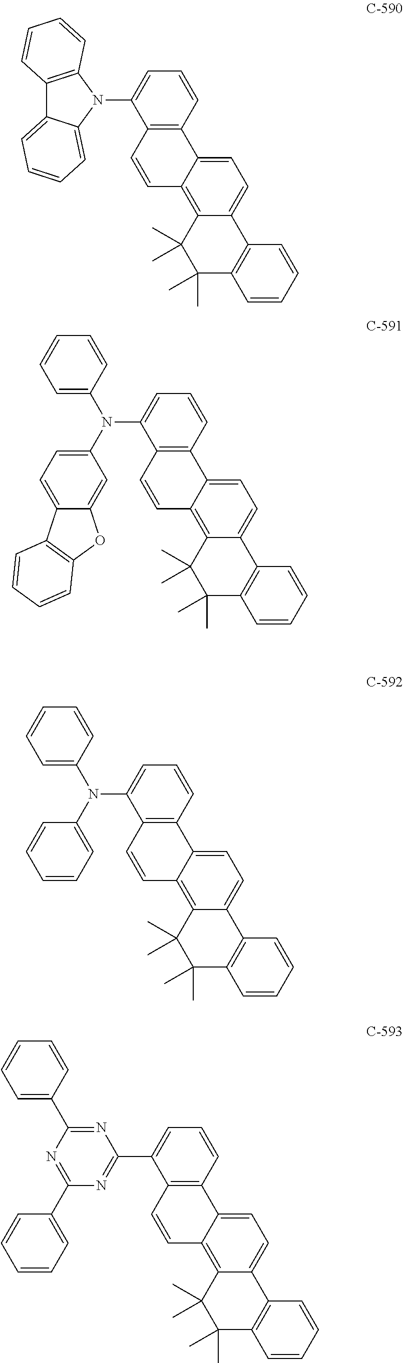 [SMILES: CC1(C)c2ccccc2-c2ccc3c(ccc4c(-n5c6ccccc6c6ccccc65)cccc43)c2C1(C)C ; CC1(C)c2ccccc2-c2ccc3c(ccc4c(N(c5ccccc5)c5ccc6c(c5)oc5ccccc56)cccc43)c2C1(C)C ; CC1(C)c2ccccc2-c2ccc3c(ccc4c(N(c5ccccc5)c5ccccc5)cccc43)c2C1(C)C ; CC1(C)c2ccccc2-c2ccc3c(ccc4c(-c5nc(-c6ccccc6)nc(-c6ccccc6)n5)cccc43)c2C1(C)C]

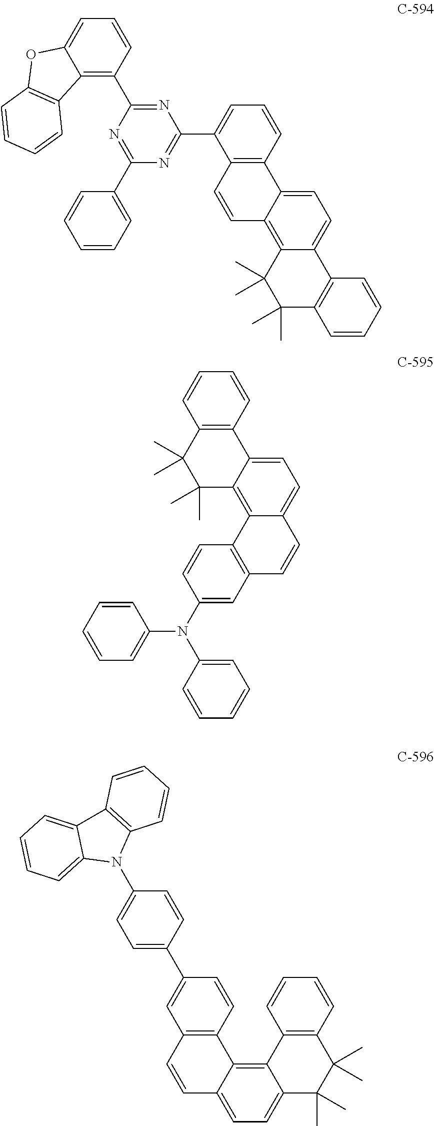 [SMILES: CC1(C)c2ccccc2-c2ccc3c(ccc4c(-c5nc(-c6ccccc6)nc(-c6cccc7oc8ccccc8c67)n5)cccc43)c2C1(C)C ; CC1(C)c2ccccc2-c2ccc3ccc4cc(N(c5ccccc5)c5ccccc5)ccc4c3c2C1(C)C ; CC1(C)c2ccccc2-c2c(ccc3ccc4cc(-c5ccc(-n6c7ccccc7c7ccccc76)cc5)ccc4c23)C1(C)C]

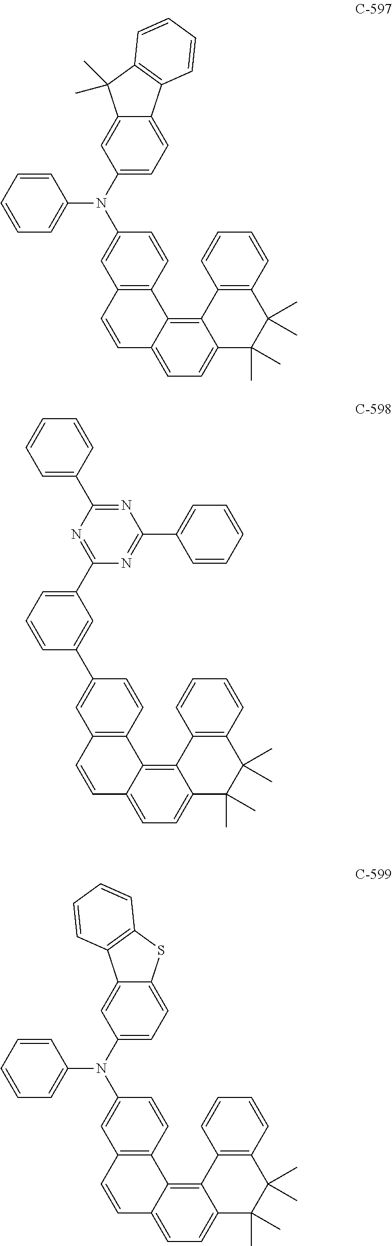 [SMILES: CC1(C)c2ccccc2-c2ccc(N(c3ccccc3)c3ccc4c(ccc5ccc6c(c54)-c4ccccc4C(C)(C)C6(C)C)c3)cc21 ; CC1(C)c2ccccc2-c2c(ccc3ccc4cc(-c5cccc(-c6nc(-c7ccccc7)nc(-c7ccccc7)n6)c5)ccc4c23)C1(C)C ; CC1(C)c2ccccc2-c2c(ccc3ccc4cc(N(c5ccccc5)c5ccc6sc7ccccc7c6c5)ccc4c23)C1(C)C]

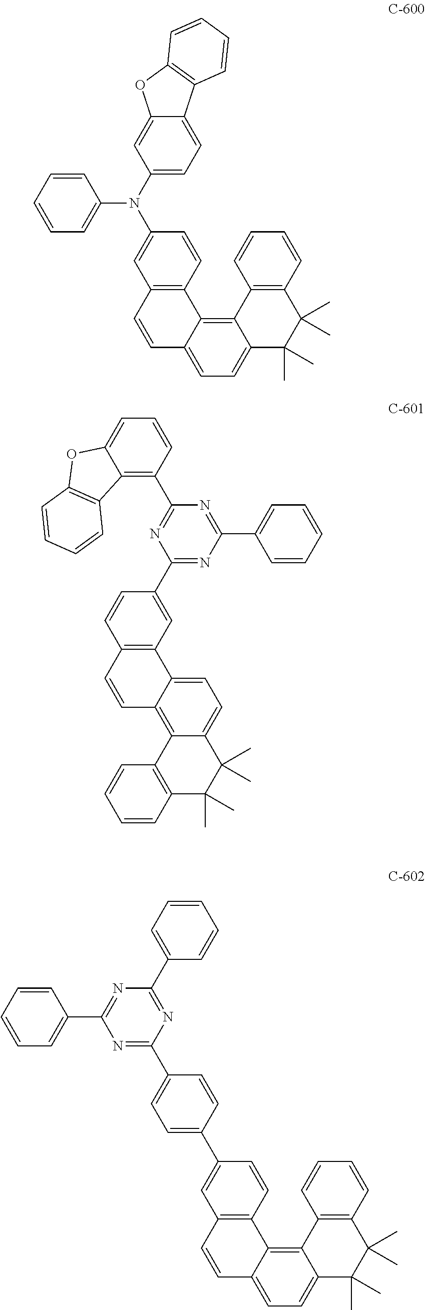 [SMILES: CC1(C)c2ccccc2-c2c(ccc3ccc4cc(N(c5ccccc5)c5ccc6c(c5)oc5ccccc56)ccc4c23)C1(C)C ; CC1(C)c2ccccc2-c2c(ccc3c2ccc2ccc(-c4nc(-c5ccccc5)nc(-c5cccc6oc7ccccc7c56)n4)cc23)C1(C)C ; CC1(C)c2ccccc2-c2c(ccc3ccc4cc(-c5ccc(-c6nc(-c7ccccc7)nc(-c7ccccc7)n6)cc5)ccc4c23)C1(C)C]

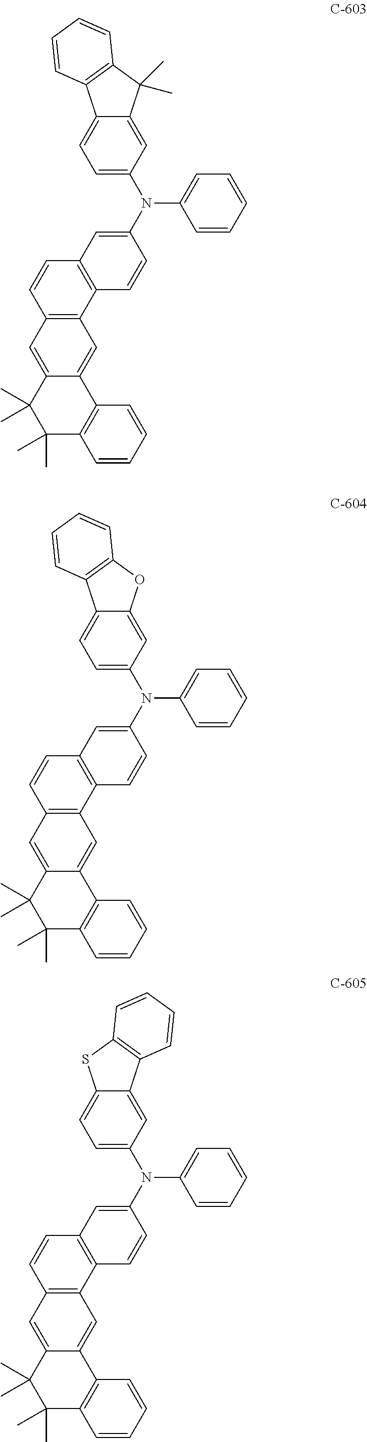

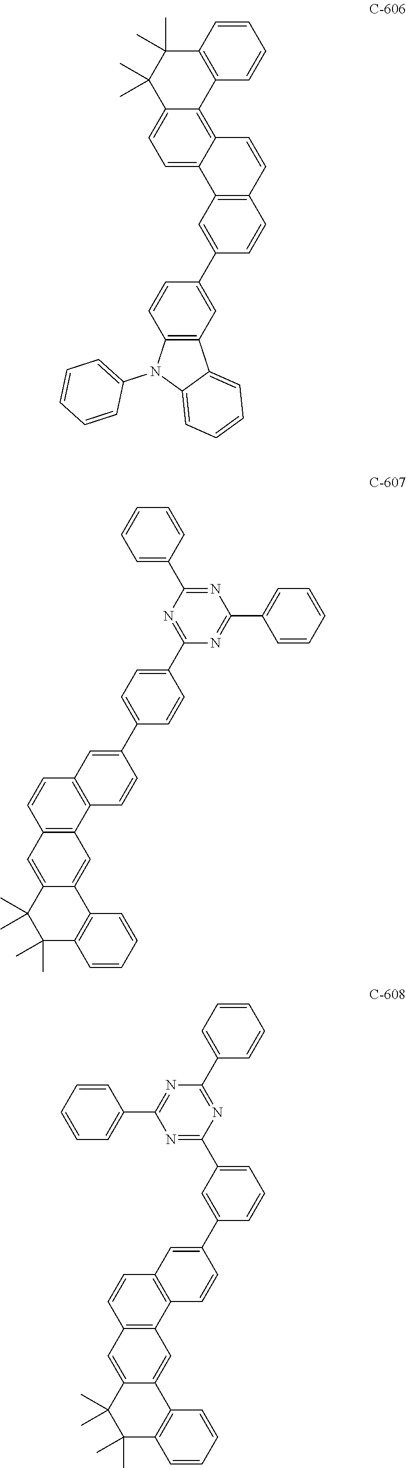 [SMILES: CC1(C)c2ccccc2-c2c(ccc3c2ccc2ccc(-c4ccc5c(c4)c4ccccc4n5-c4ccccc4)cc23)C1(C)C ; CC1(C)c2ccccc2-c2cc3c(ccc4cc(-c5ccc(-c6nc(-c7ccccc7)nc(-c7ccccc7)n6)cc5)ccc43)cc2C1(C)C ; CC1(C)c2ccccc2-c2cc3c(ccc4cc(-c5cccc(-c6nc(-c7ccccc7)nc(-c7ccccc7)n6)c5)ccc43)cc2C1(C)C]

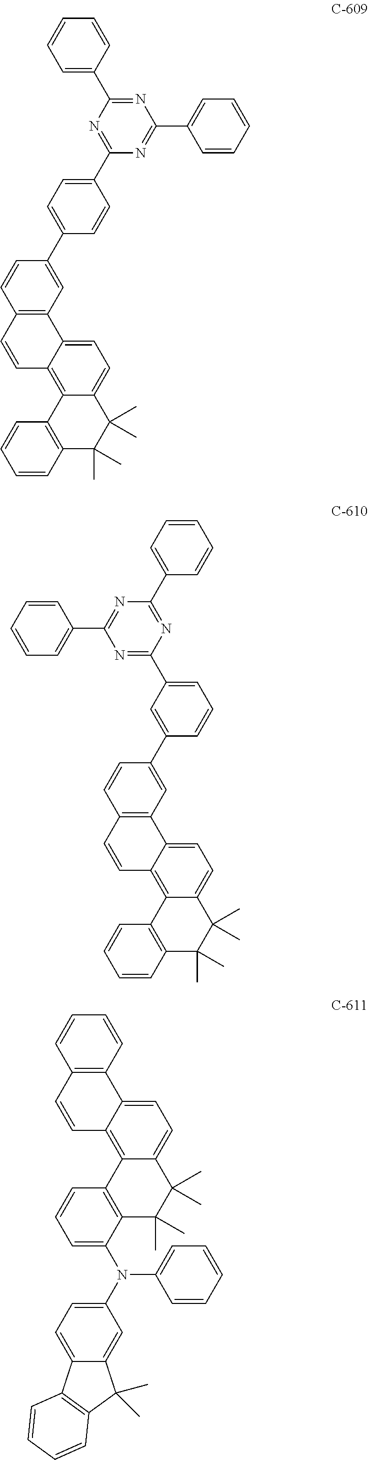

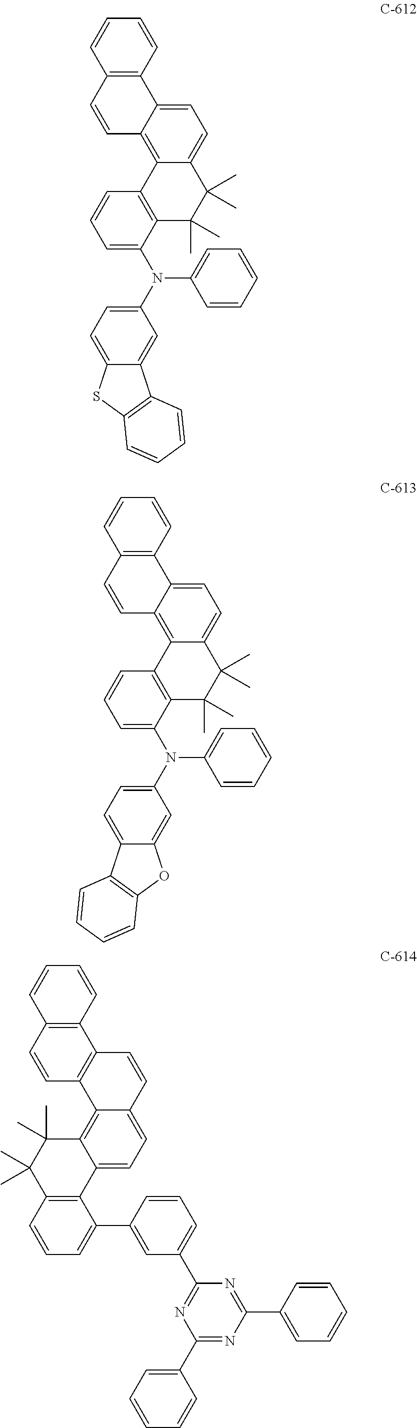

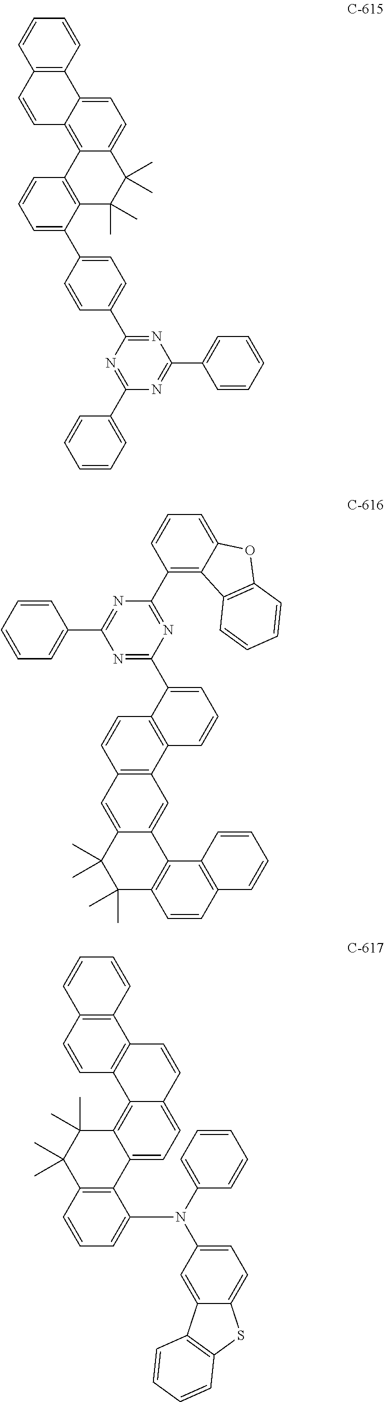

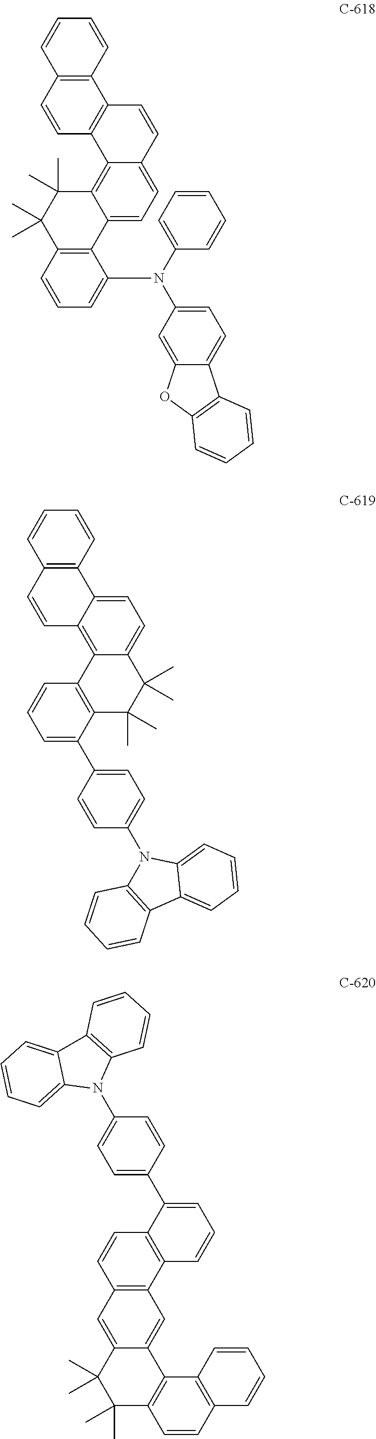 [SMILES: CC1(C)c2cccc(N(c3ccccc3)c3ccc4c(c3)oc3ccccc34)c2-c2ccc3ccc4c5ccccc5ccc4c3c2C1(C)C ; CC1(C)c2ccc3c(ccc4ccccc43)c2-c2cccc(-c3ccc(-n4c5ccccc5c5ccccc54)cc3)c2C1(C)C ; CC1(C)c2cc3ccc4c(-c5ccc(-n6c7ccccc7c7ccccc76)cc5)cccc4c3cc2-c2c(ccc3ccccc23)C1(C)C]

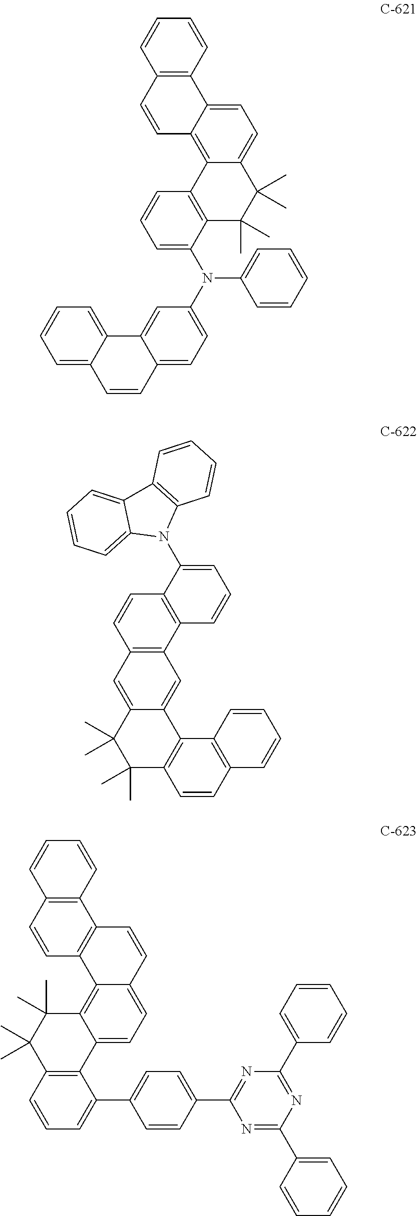 [SMILES: CC1(C)c2ccc3c(ccc4ccccc43)c2-c2cccc(N(c3ccccc3)c3ccc4ccc5ccccc5c4c3)c2C1(C)C ; CC1(C)c2cc3ccc4c(-n5c6ccccc6c6ccccc65)cccc4c3cc2-c2c(ccc3ccccc23)C1(C)C ; CC1(C)c2cccc(-c3ccc(-c4nc(-c5ccccc5)nc(-c5ccccc5)n4)cc3)c2-c2ccc3ccc4c5ccccc5ccc4c3c2C1(C)C]

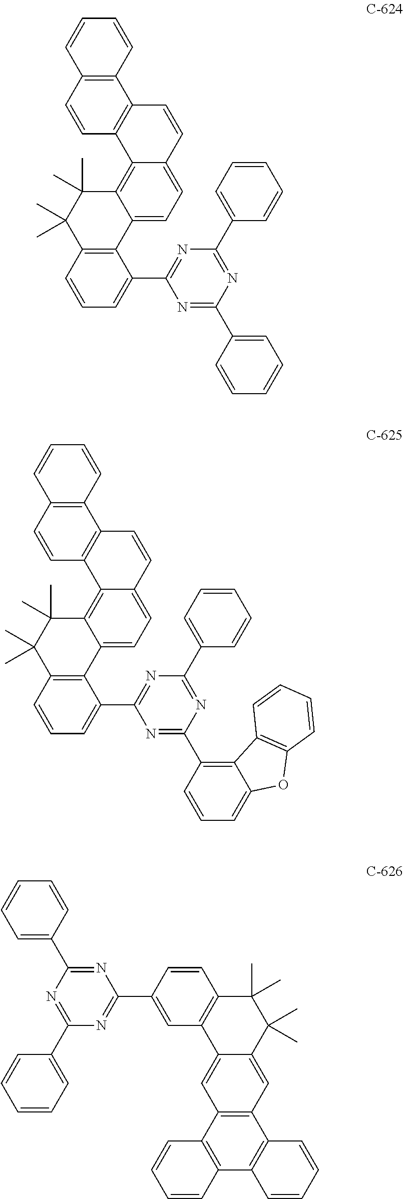 [SMILES: CC1(C)c2cccc(-c3nc(-c4ccccc4)nc(-c4ccccc4)n3)c2-c2ccc3ccc4c5ccccc5ccc4c3c2C1(C)C ; CC1(C)c2cccc(-c3nc(-c4ccccc4)nc(-c4cccc5oc6ccccc6c45)n3)c2-c2ccc3ccc4c5ccccc5ccc4c3c2C1(C)C ; CC1(C)c2ccc(-c3nc(-c4ccccc4)nc(-c4ccccc4)n3)cc2-c2cc3c4ccccc4c4ccccc4c3cc2C1(C)C]

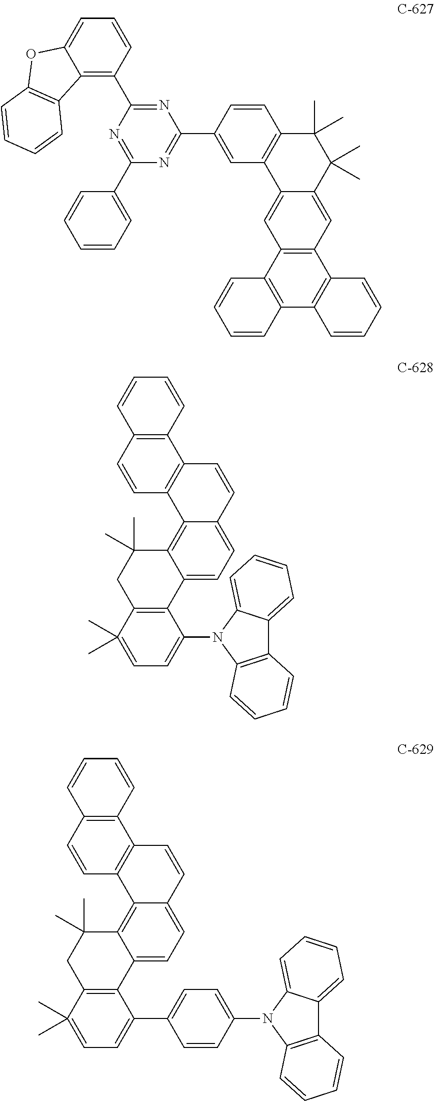

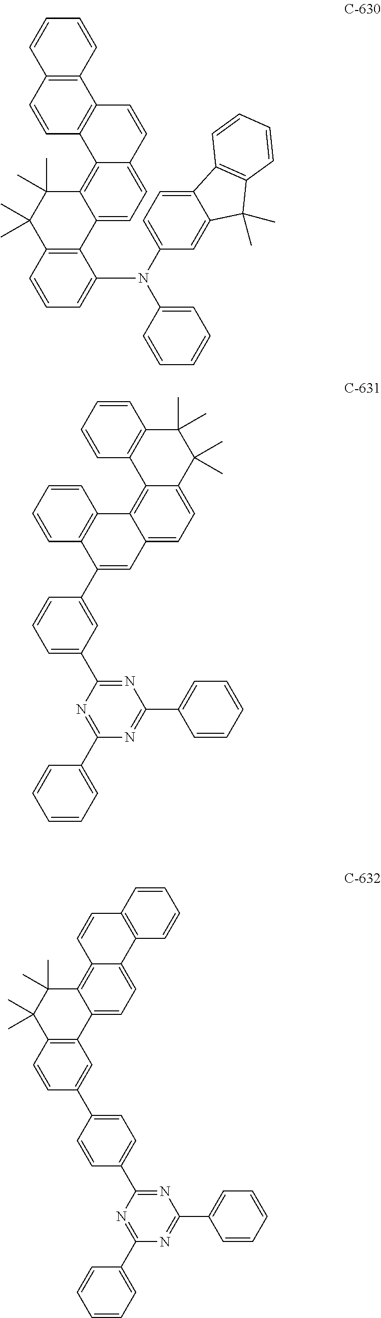

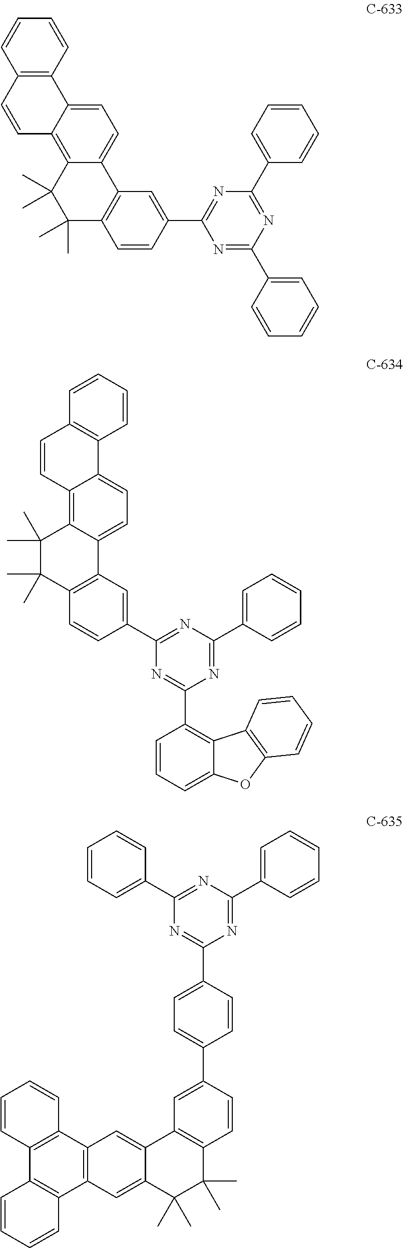 [SMILES: CC1(C)c2ccc(-c3nc(-c4ccccc4)nc(-c4ccccc4)n3)cc2-c2ccc3c(ccc4ccccc43)c2C1(C)C ; CC1(C)c2ccc(-c3nc(-c4ccccc4)nc(-c4cccc5oc6ccccc6c45)n3)cc2-c2ccc3c(ccc4ccccc43)c2C1(C)C ; CC1(C)c2ccc(-c3ccc(-c4nc(-c5ccccc5)nc(-c5ccccc5)n4)cc3)cc2-c2cc3c4ccccc4c4ccccc4c3cc2C1(C)C]

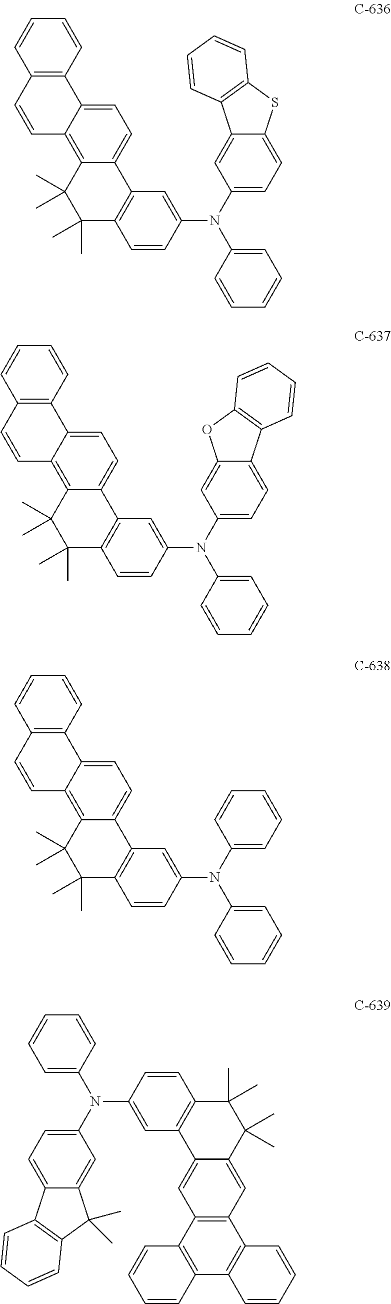 [SMILES: CC1(C)c2ccc(N(c3ccccc3)c3ccc4sc5ccccc5c4c3)cc2-c2ccc3c(ccc4ccccc43)c2C1(C)C ; CC1(C)c2ccc(N(c3ccccc3)c3ccc4c(c3)oc3ccccc34)cc2-c2ccc3c(ccc4ccccc43)c2C1(C)C ; CC1(C)c2ccc(N(c3ccccc3)c3ccccc3)cc2-c2ccc3c(ccc4ccccc43)c2C1(C)C ; CC1(C)c2ccccc2-c2ccc(N(c3ccccc3)c3ccc4c(c3)-c3cc5c6ccccc6c6ccccc6c5cc3C(C)(C)C4(C)C)cc21]

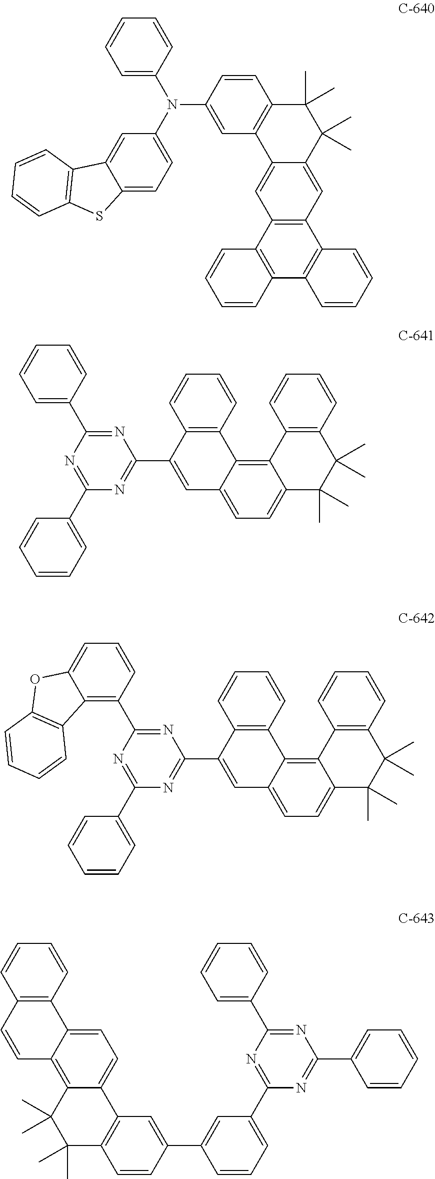 [SMILES: CC1(C)c2ccc(N(c3ccccc3)c3ccc4sc5ccccc5c4c3)cc2-c2cc3c4ccccc4c4ccccc4c3cc2C1(C)C ; CC1(C)c2ccccc2-c2c(ccc3cc(-c4nc(-c5ccccc5)nc(-c5ccccc5)n4)c4ccccc4c23)C1(C)C ; CC1(C)c2ccccc2-c2c(ccc3cc(-c4nc(-c5ccccc5)nc(-c5cccc6oc7ccccc7c56)n4)c4ccccc4c23)C1(C)C ; CC1(C)c2ccc(-c3cccc(-c4nc(-c5ccccc5)nc(-c5ccccc5)n4)c3)cc2-c2ccc3c(ccc4ccccc43)c2C1(C)C]

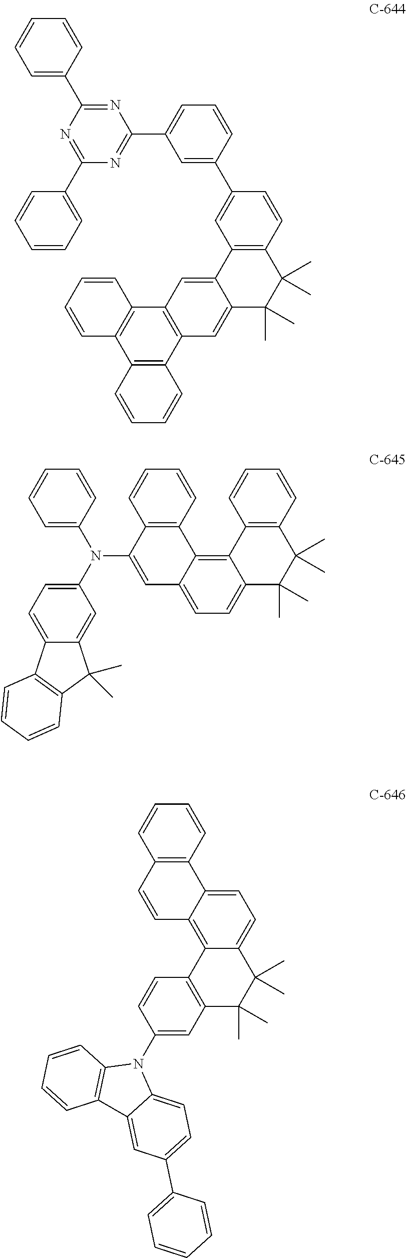 [SMILES: CC1(C)c2ccc(-c3cccc(-c4nc(-c5ccccc5)nc(-c5ccccc5)n4)c3)cc2-c2cc3c4ccccc4c4ccccc4c3cc2C1(C)C ; CC1(C)c2ccccc2-c2ccc(N(c3ccccc3)c3cc4ccc5c(c4c4ccccc34)-c3ccccc3C(C)(C)C5(C)C)cc21 ; CC1(C)c2cc(-n3c4ccccc4c4cc(-c5ccccc5)ccc43)ccc2-c2c(ccc3c2ccc2ccccc23)C1(C)C]

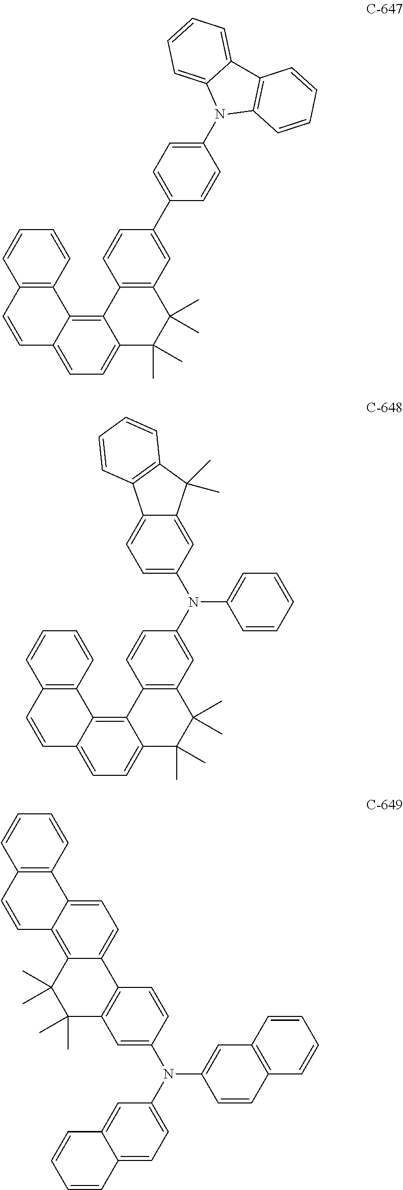 [SMILES: CC1(C)c2cc(-c3ccc(-n4c5ccccc5c5ccccc54)cc3)ccc2-c2c(ccc3ccc4ccccc4c23)C1(C)C ; CC1(C)c2ccccc2-c2ccc(N(c3ccccc3)c3ccc4c(c3)C(C)(C)C(C)(C)c3ccc5ccc6ccccc6c5c3-4)cc21 ; CC1(C)c2cc(N(c3ccc4ccccc4c3)c3ccc4ccccc4c3)ccc2-c2ccc3c(ccc4ccccc43)c2C1(C)C]

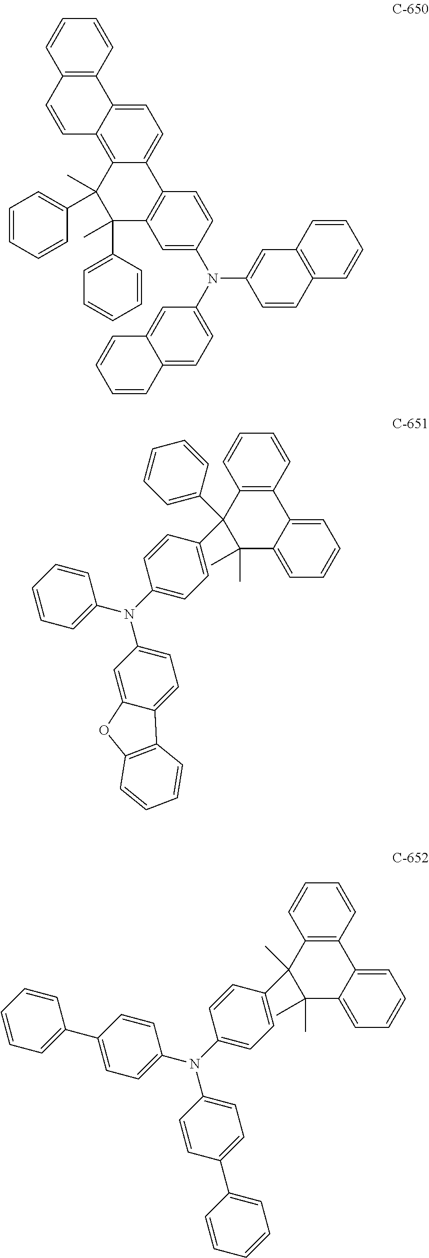 [SMILES: CC1(c2ccccc2)c2cc(N(c3ccc4ccccc4c3)c3ccc4ccccc4c3)ccc2-c2ccc3c(ccc4ccccc43)c2C1(C)c1ccccc1 ; CC1(C)c2ccccc2-c2ccccc2C1(c1ccccc1)c1ccc(N(c2ccccc2)c2ccc3c(c2)oc2ccccc23)cc1 ; CC1(C)c2ccccc2-c2ccccc2C1(C)c1ccc(N(c2ccc(-c3ccccc3)cc2)c2ccc(-c3ccccc3)cc2)cc1]

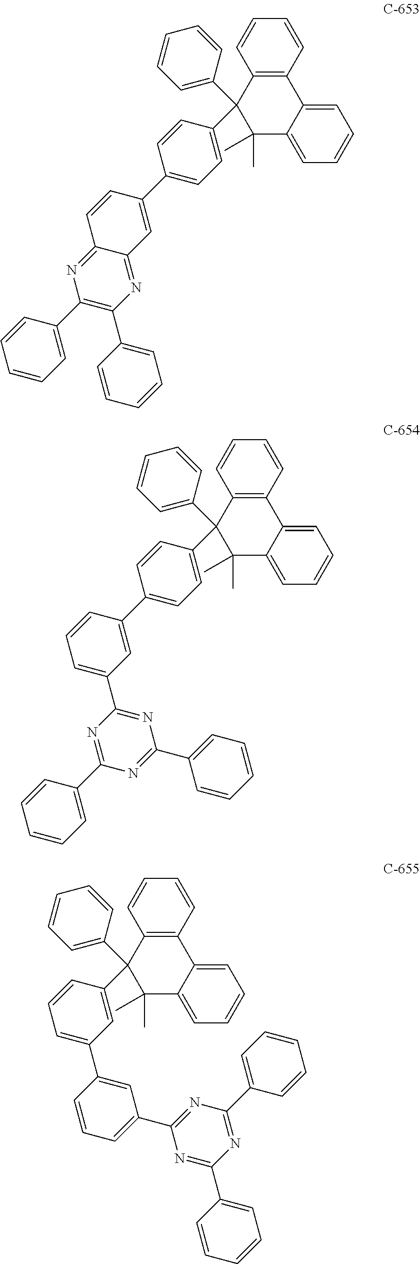 [SMILES: CC1(C)c2ccccc2-c2ccccc2C1(c1ccccc1)c1ccc(-c2ccc3nc(-c4ccccc4)c(-c4ccccc4)nc3c2)cc1 ; CC1(C)c2ccccc2-c2ccccc2C1(c1ccccc1)c1ccc(-c2cccc(-c3nc(-c4ccccc4)nc(-c4ccccc4)n3)c2)cc1 ; CC1(C)c2ccccc2-c2ccccc2C1(c1ccccc1)c1cccc(-c2cccc(-c3nc(-c4ccccc4)nc(-c4ccccc4)n3)c2)c1]

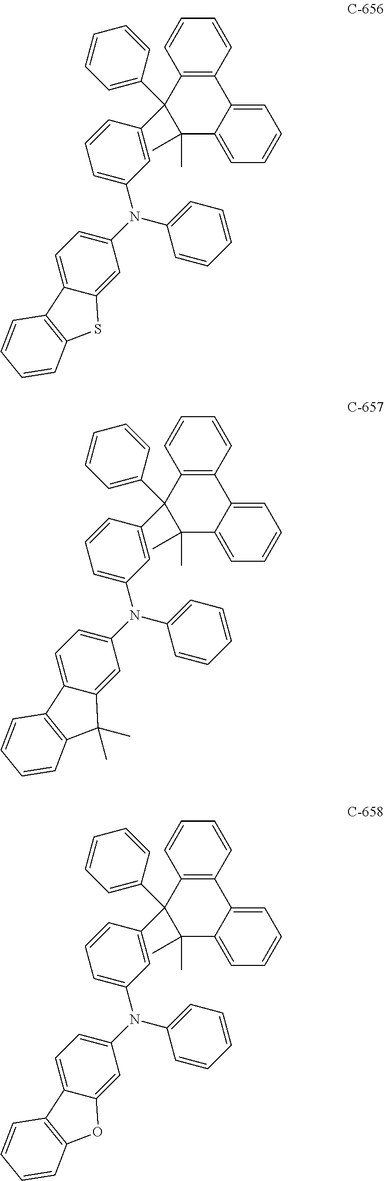 [SMILES: CC1(C)c2ccccc2-c2ccccc2C1(c1ccccc1)c1cccc(N(c2ccccc2)c2ccc3c(c2)sc2ccccc23)c1 ; CC1(C)c2ccccc2-c2ccc(N(c3ccccc3)c3cccc(C4(c5ccccc5)c5ccccc5-c5ccccc5C4(C)C)c3)cc21 ; CC1(C)c2ccccc2-c2ccccc2C1(c1ccccc1)c1cccc(N(c2ccccc2)c2ccc3c(c2)oc2ccccc23)c1]

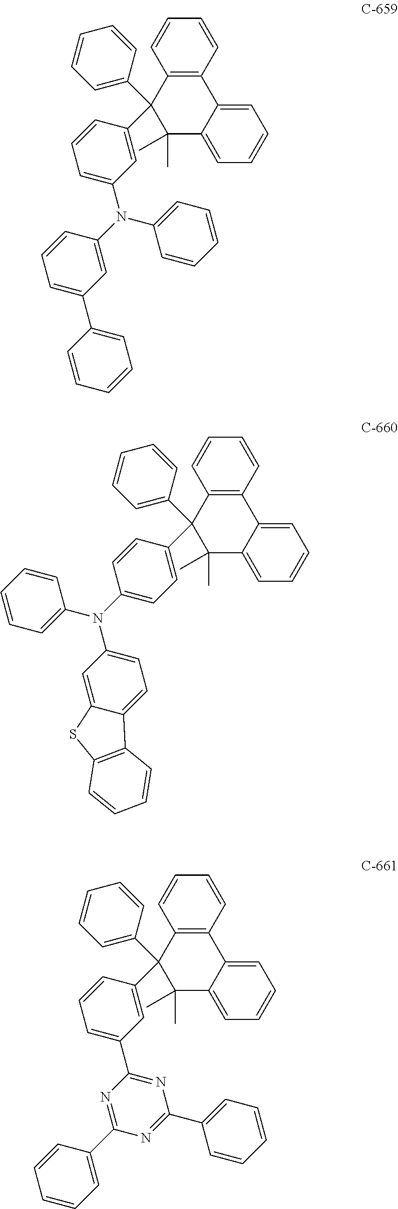 [SMILES: CC1(C)c2ccccc2-c2ccccc2C1(c1ccccc1)c1cccc(N(c2ccccc2)c2cccc(-c3ccccc3)c2)c1 ; CC1(C)c2ccccc2-c2ccccc2C1(c1ccccc1)c1ccc(N(c2ccccc2)c2ccc3c(c2)sc2ccccc23)cc1 ; CC1(C)c2ccccc2-c2ccccc2C1(c1ccccc1)c1cccc(-c2nc(-c3ccccc3)nc(-c3ccccc3)n2)c1]

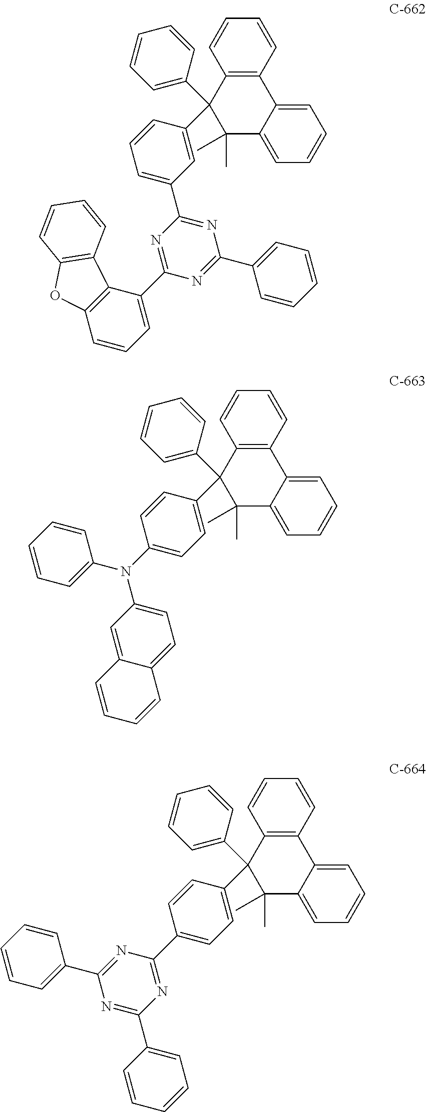 [SMILES: CC1(C)c2ccccc2-c2ccccc2C1(c1ccccc1)c1cccc(-c2nc(-c3ccccc3)nc(-c3cccc4oc5ccccc5c34)n2)c1 ; CC1(C)c2ccccc2-c2ccccc2C1(c1ccccc1)c1ccc(N(c2ccccc2)c2ccc3ccccc3c2)cc1 ; CC1(C)c2ccccc2-c2ccccc2C1(c1ccccc1)c1ccc(-c2nc(-c3ccccc3)nc(-c3ccccc3)n2)cc1]

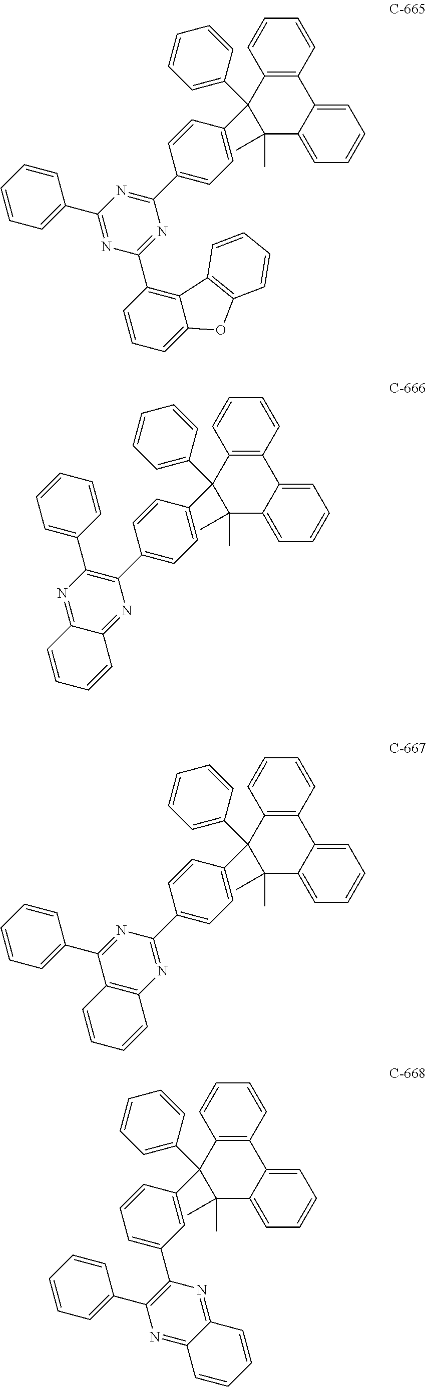 [SMILES: CC1(C)c2ccccc2-c2ccccc2C1(c1ccccc1)c1ccc(-c2nc(-c3ccccc3)nc(-c3cccc4oc5ccccc5c34)n2)cc1 ; CC1(C)c2ccccc2-c2ccccc2C1(c1ccccc1)c1ccc(-c2nc3ccccc3nc2-c2ccccc2)cc1 ; CC1(C)c2ccccc2-c2ccccc2C1(c1ccccc1)c1ccc(-c2nc(-c3ccccc3)c3ccccc3n2)cc1 ; CC1(C)c2ccccc2-c2ccccc2C1(c1ccccc1)c1cccc(-c2nc3ccccc3nc2-c2ccccc2)c1]

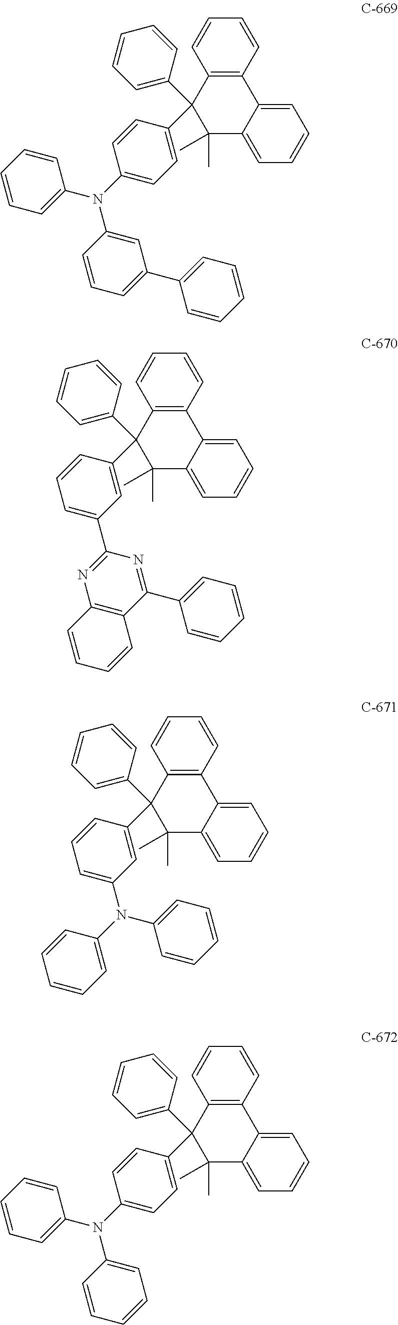

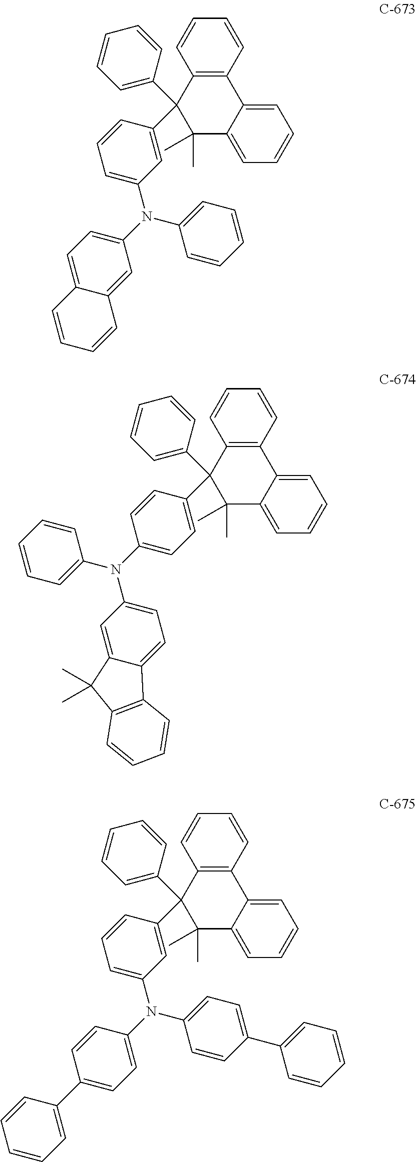 [SMILES: CC1(C)c2ccccc2-c2ccccc2C1(c1ccccc1)c1cccc(N(c2ccccc2)c2ccc3ccccc3c2)c1 ; CC1(C)c2ccccc2-c2ccc(N(c3ccccc3)c3ccc(C4(c5ccccc5)c5ccccc5-c5ccccc5C4(C)C)cc3)cc21 ; CC1(C)c2ccccc2-c2ccccc2C1(c1ccccc1)c1cccc(N(c2ccc(-c3ccccc3)cc2)c2ccc(-c3ccccc3)cc2)c1]

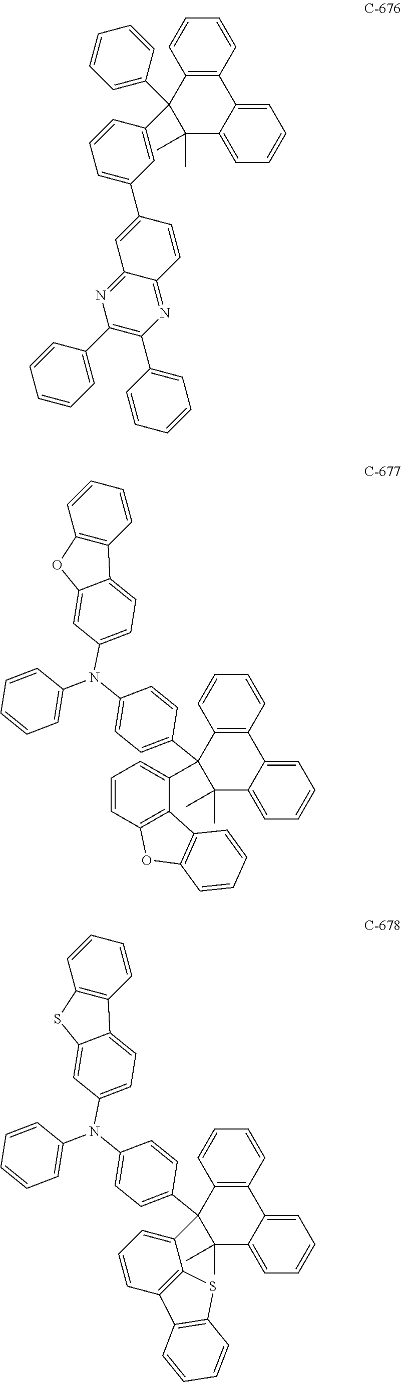 [SMILES: CC1(C)c2ccccc2-c2ccccc2C1(c1ccccc1)c1cccc(-c2ccc3nc(-c4ccccc4)c(-c4ccccc4)nc3c2)c1 ; CC1(C)c2ccccc2-c2ccccc2C1(c1ccc(N(c2ccccc2)c2ccc3c(c2)oc2ccccc23)cc1)c1cccc2oc3ccccc3c12 ; CC1(C)c2ccccc2-c2ccccc2C1(c1ccc(N(c2ccccc2)c2ccc3c(c2)sc2ccccc23)cc1)c1cccc2c1sc1ccccc12]

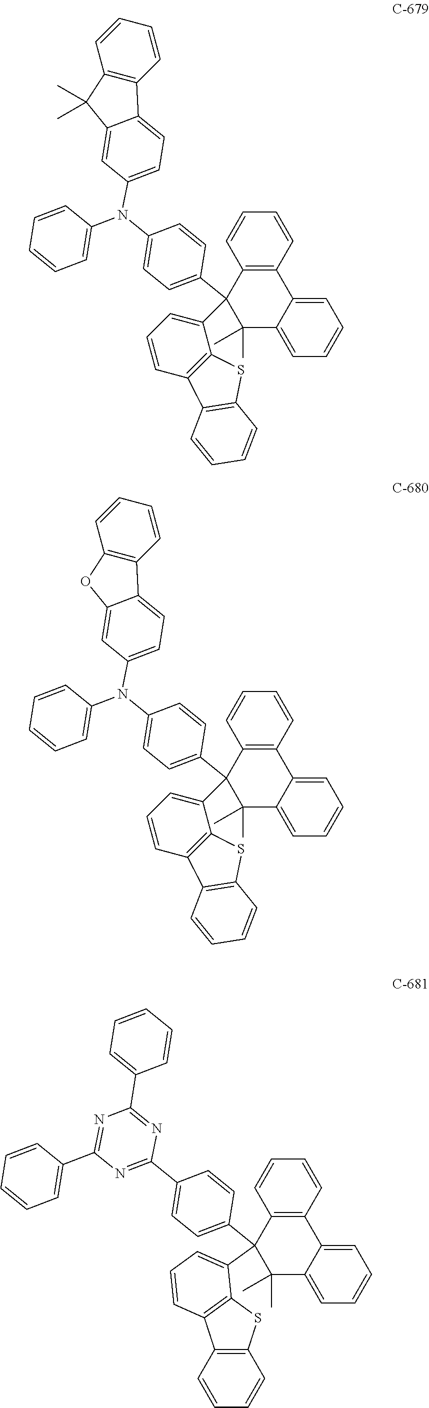 [SMILES: CC1(C)c2ccccc2-c2ccc(N(c3ccccc3)c3ccc(C4(c5cccc6c5sc5ccccc56)c5ccccc5-c5ccccc5C4(C)C)cc3)cc21 ; CC1(C)c2ccccc2-c2ccccc2C1(c1ccc(N(c2ccccc2)c2ccc3c(c2)oc2ccccc23)cc1)c1cccc2c1sc1ccccc12 ; CC1(C)c2ccccc2-c2ccccc2C1(c1ccc(-c2nc(-c3ccccc3)nc(-c3ccccc3)n2)cc1)c1cccc2c1sc1ccccc12]

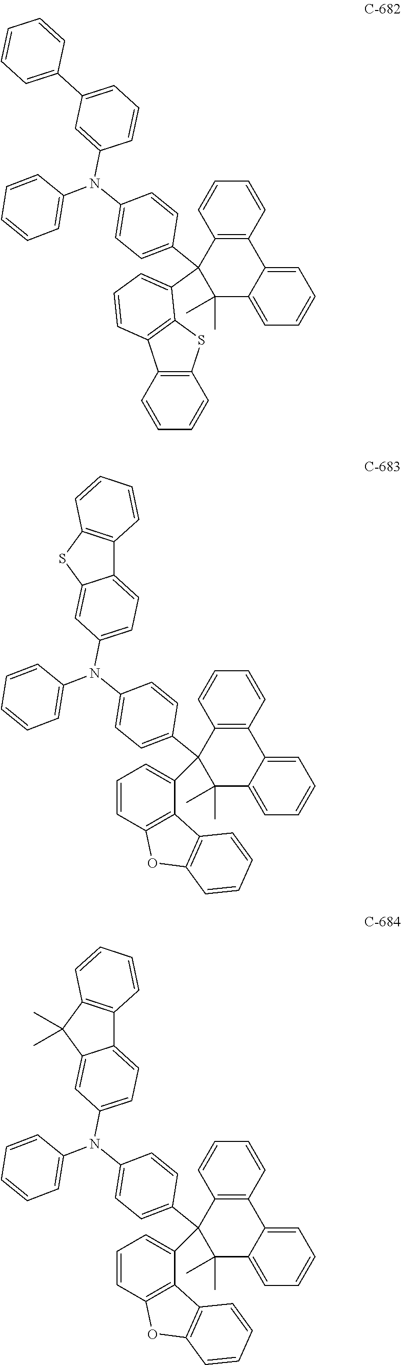 [SMILES: CC1(C)c2ccccc2-c2ccccc2C1(c1ccc(N(c2ccccc2)c2cccc(-c3ccccc3)c2)cc1)c1cccc2c1sc1ccccc12 ; CC1(C)c2ccccc2-c2ccccc2C1(c1ccc(N(c2ccccc2)c2ccc3c(c2)sc2ccccc23)cc1)c1cccc2oc3ccccc3c12 ; CC1(C)c2ccccc2-c2ccc(N(c3ccccc3)c3ccc(C4(c5cccc6oc7ccccc7c56)c5ccccc5-c5ccccc5C4(C)C)cc3)cc21]

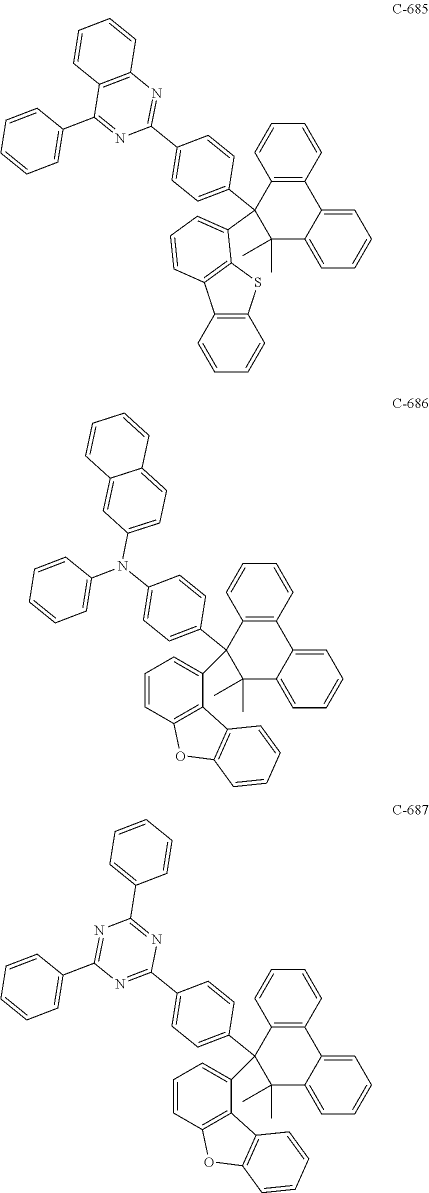 [SMILES: CC1(C)c2ccccc2-c2ccccc2C1(c1ccc(-c2nc(-c3ccccc3)c3ccccc3n2)cc1)c1cccc2c1sc1ccccc12 ; CC1(C)c2ccccc2-c2ccccc2C1(c1ccc(N(c2ccccc2)c2ccc3ccccc3c2)cc1)c1cccc2oc3ccccc3c12 ; CC1(C)c2ccccc2-c2ccccc2C1(c1ccc(-c2nc(-c3ccccc3)nc(-c3ccccc3)n2)cc1)c1cccc2oc3ccccc3c12]

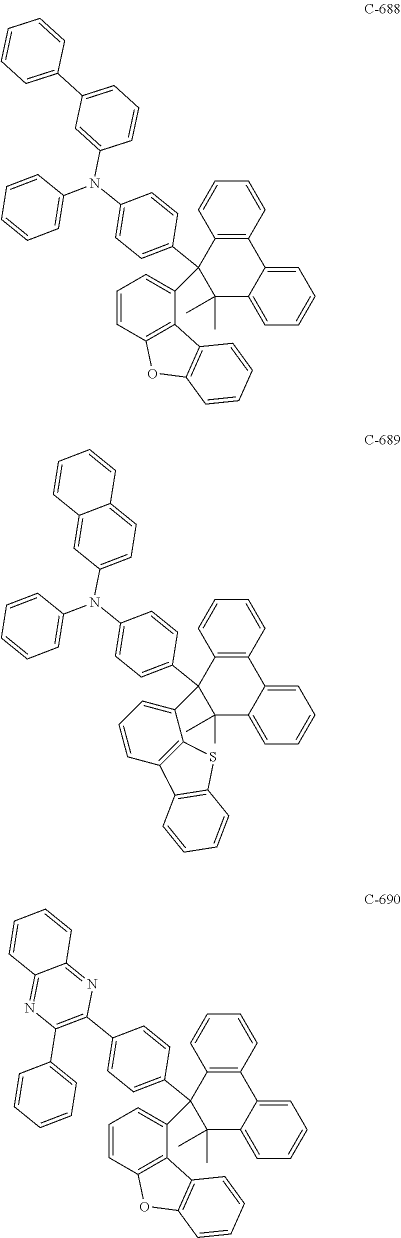 [SMILES: CC1(C)c2ccccc2-c2ccccc2C1(c1ccc(N(c2ccccc2)c2cccc(-c3ccccc3)c2)cc1)c1cccc2oc3ccccc3c12 ; CC1(C)c2ccccc2-c2ccccc2C1(c1ccc(N(c2ccccc2)c2ccc3ccccc3c2)cc1)c1cccc2c1sc1ccccc12 ; CC1(C)c2ccccc2-c2ccccc2C1(c1ccc(-c2nc3ccccc3nc2-c2ccccc2)cc1)c1cccc2oc3ccccc3c12]

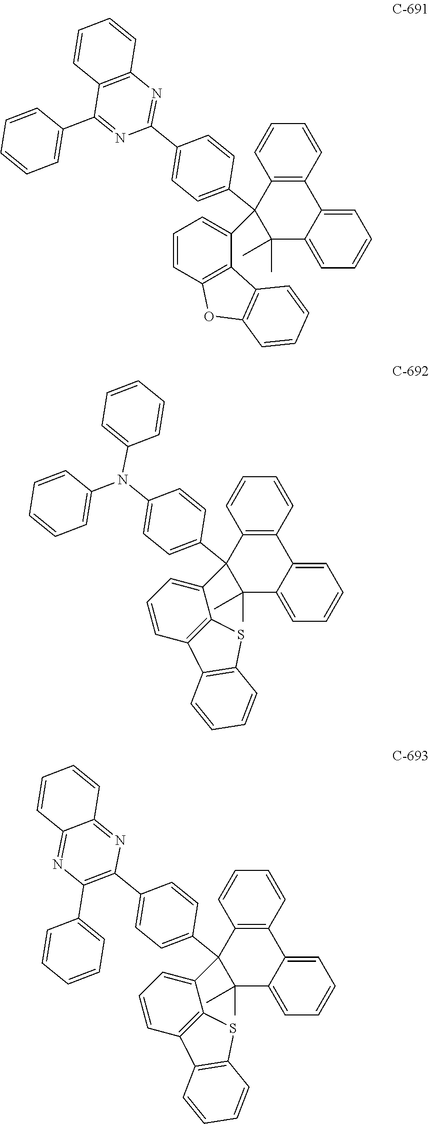 [SMILES: CC1(C)c2ccccc2-c2ccccc2C1(c1ccc(-c2nc(-c3ccccc3)c3ccccc3n2)cc1)c1cccc2oc3ccccc3c12 ; CC1(C)c2ccccc2-c2ccccc2C1(c1ccc(N(c2ccccc2)c2ccccc2)cc1)c1cccc2c1sc1ccccc12 ; CC1(C)c2ccccc2-c2ccccc2C1(c1ccc(-c2nc3ccccc3nc2-c2ccccc2)cc1)c1cccc2c1sc1ccccc12]

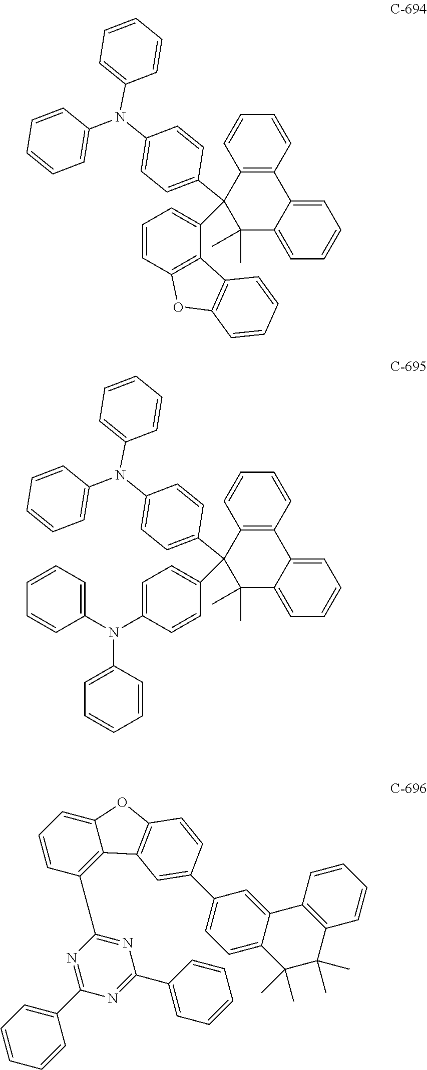 [SMILES: CC1(C)c2ccccc2-c2ccccc2C1(c1ccc(N(c2ccccc2)c2ccccc2)cc1)c1cccc2oc3ccccc3c12 ; CC1(C)c2ccccc2-c2ccccc2C1(c1ccc(N(c2ccccc2)c2ccccc2)cc1)c1ccc(N(c2ccccc2)c2ccccc2)cc1 ; CC1(C)c2ccccc2-c2cc(-c3ccc4oc5cccc(-c6nc(-c7ccccc7)nc(-c7ccccc7)n6)c5c4c3)ccc2C1(C)C]

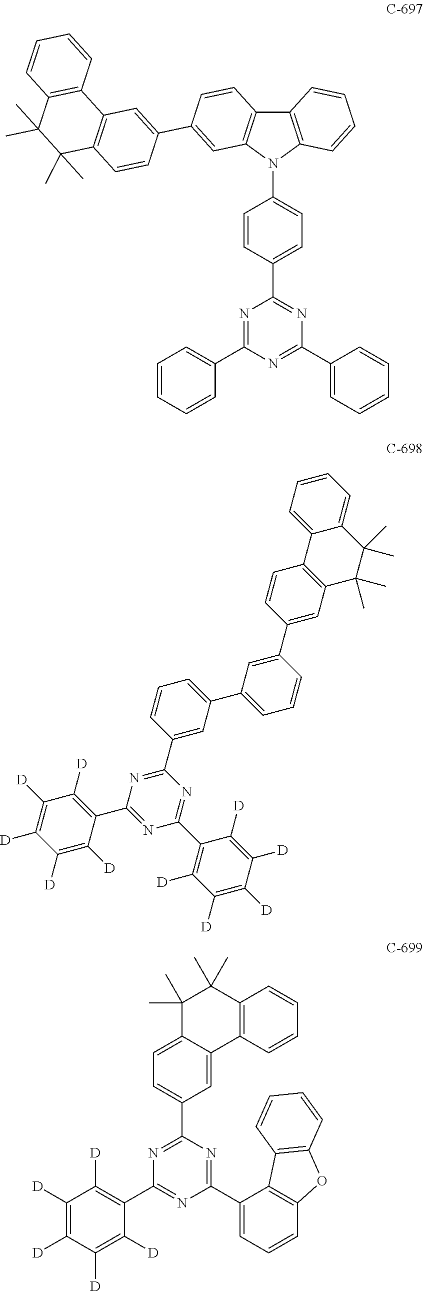

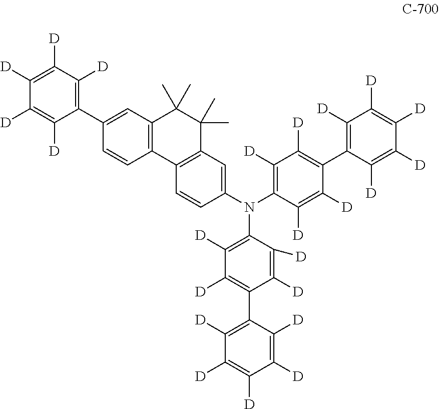 [SMILES: [2H]c1c([2H])c([2H])c(-c2ccc3c(c2)C(C)(C)C(C)(C)c2cc(N(c4c([2H])c([2H])c(-c5c([2H])c([2H])c([2H])c([2H])c5[2H])c([2H])c4[2H])c4c([2H])c([2H])c(-c5c([2H])c([2H])c([2H])c([2H])c5[2H])c([2H])c4[2H])ccc2-3)c([2H])c1[2H]]

The organic electroluminescent compound of formula 1 according to the present disclosure may be produced as represented by the following reaction schemes 1 to 3, but is not limited thereto. Further, it may be prepared by a synthetic method known to a person skilled in the art.

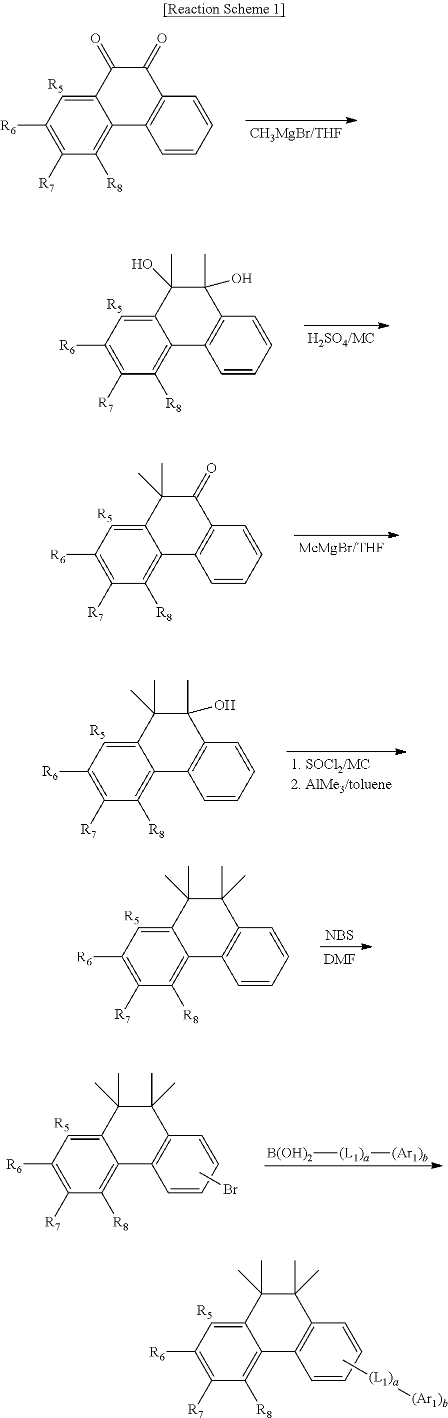

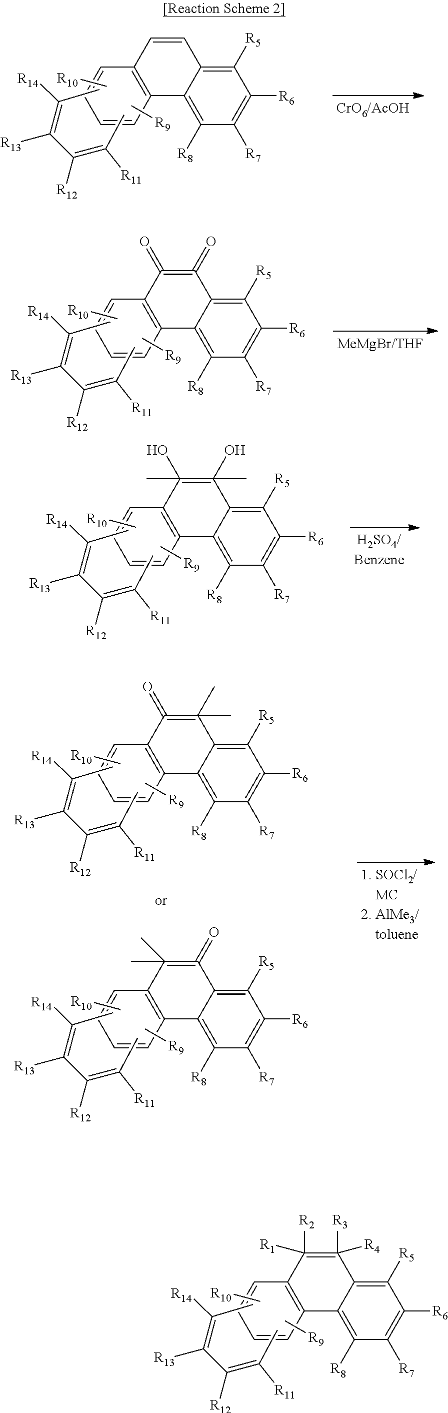

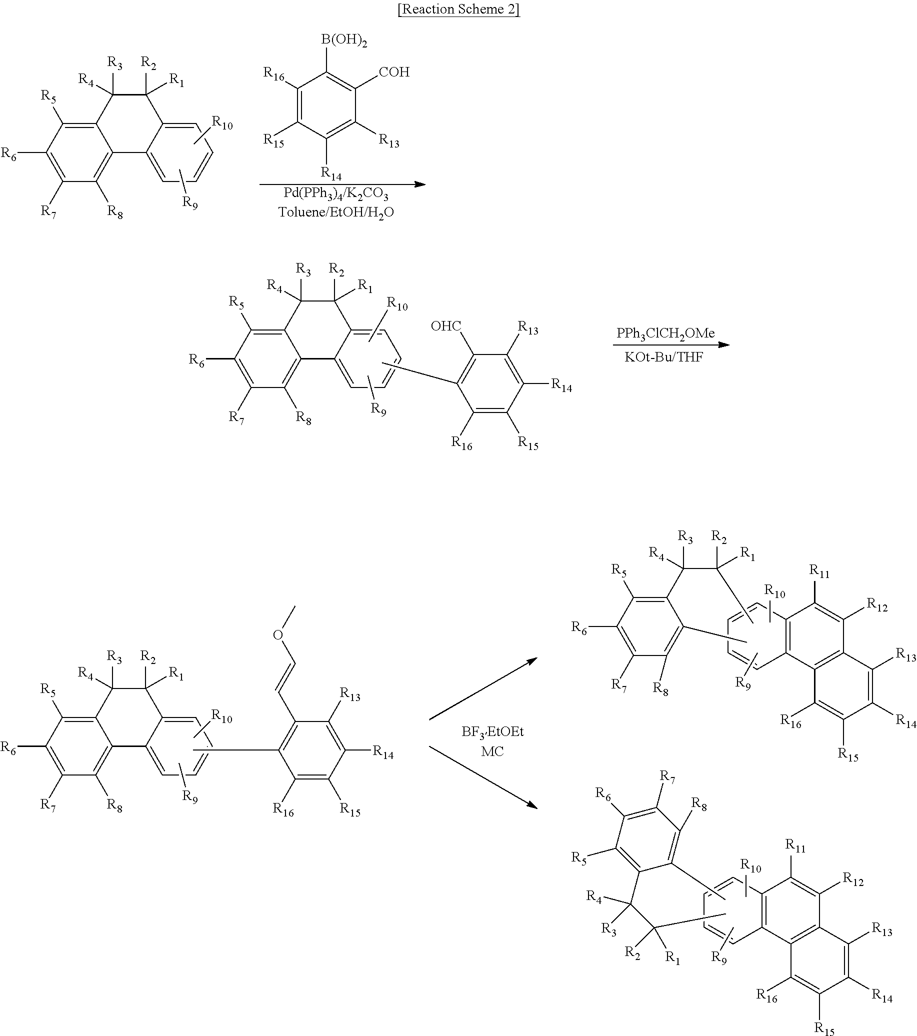

In reaction schemes 1 to 3 above, R₁ to R₁₂, L₁, and Ar₁ are as defined in formula 1 above, and R₁₃ to R₁₆ are as defined as R₅ to R₁₂ in formula 1 above.

As described above, exemplary synthesis examples of the compounds represented by formula 1 according to the present disclosure are described, but they are based on Suzuki cross-coupling reaction, Buchwald-Hartwig cross coupling reaction, N-arylation reaction, H-mont-mediated etherification reaction, Miyaura borylation reaction, Intramolecular acid-induced cyclization reaction, Pd(II)-catalyzed oxidative cyclization reaction, Grignard reaction, Heck reaction, Cyclic Dehydration reaction, SN₁ substitution reaction, SN₂ substitution reaction, and Phosphine-mediated reductive cyclization reaction, etc. It will be understood by one skilled in the art that the above reaction proceeds even if other substituents defined in formula 1, other than the substituents described in the specific synthesis examples, are bonded.

The organic electroluminescent compound according to another embodiment may be represented by the following formula 2.

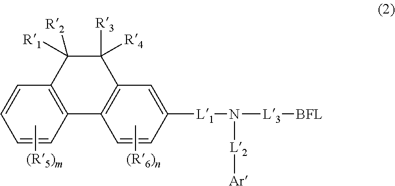

In formula 2,

R′₁ to R′₄ each independently represent hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent(s) to form a ring(s);

R′₅ and R′₆ each independently represent hydrogen or deuterium;

L′₁ to L′₃ each independently represent a single bond, a substituted or unsubstituted (C6-C30)arylene, or a substituted or unsubstituted (3- to 30-membered)heteroarylene;

Ar′ represents a substituted or unsubstituted (C6-C30)aryl, or a substituted or unsubstituted (3- to 30-membered)heteroaryl;

BFL represents a substituted or unsubstituted benzo[a]fluorenyl, a substituted or unsubstituted benzo[b]fluorenyl, or a substituted or unsubstituted benzo[c]fluorenyl; and m represents an integer of 1 to 4, n represents an integer of 1 to 3, and when m and n are 2 or more, each of R's and each of R's may be the same or different.

In one embodiment, R′₁ to R′₄ each independently may be hydrogen, deuterium, a substituted or unsubstituted (C1-C30)alkyl, or a substituted or unsubstituted (C6-C30)aryl, preferably, a substituted or unsubstituted (C1-C10)alkyl, or a substituted or unsubstituted (C6-C25)aryl, more preferably, a substituted or unsubstituted (C1-C4)alkyl. For example, all of R′₁ to R′₄ may be methyl.

In one embodiment, all of R′₅ and R′₆ may be hydrogen or all of R′₅ and R′₆ may be deuterium.

In one embodiment, L′₁ to L′₃ each independently may be a single bond, a substituted or unsubstituted (C6-C30)arylene, or a substituted or unsubstituted (5- to 30-membered)heteroarylene, preferably, a single bond, a substituted or unsubstituted (C6-C25)arylene, or a substituted or unsubstituted (5- to 25-membered)heteroarylene, more preferably, a single bond, a substituted or unsubstituted (C6-C18)arylene, or a substituted or unsubstituted (5- to 18-membered)heteroarylene. For example, L′₁ to L′₃ each independently may be a single bond, or a substituted or unsubstituted phenylene, or a substituted or unsubstituted carbazolylene.

In one embodiment, Ar′ may be a substituted or unsubstituted (C6-C30)aryl, or a substituted or unsubstituted (5- to 30-membered)heteroaryl, preferably, a substituted or unsubstituted (C6-C25)aryl, or a substituted or unsubstituted (5- to 25-membered)heteroaryl, more preferably, a substituted or unsubstituted (C6-C18)aryl, or a substituted or unsubstituted (5- to 18-membered)heteroaryl. For example, Ar′ may be a substituted or unsubstituted phenyl, a substituted or unsubstituted naphthyl, p-biphenyl unsubstituted or substituted with deuterium, a substituted or unsubstituted fluorenyl, a substituted or unsubstituted carbazolyl, a substituted or unsubstituted dibenzothiophenyl, or a substituted or unsubstituted dibenzofuranyl.

In one embodiment, BFL may be a substituted or unsubstituted benzo[a]fluorenyl, a substituted or unsubstituted benzo[b]fluorenyl, or a substituted or unsubstituted benzo[c]fluorenyl, and wherein the substituent of the substituted benzo[a]fluorenyl, the substituted benzo[b]fluorenyl, or the substituted benzo[c]fluorenyl may be deuterium. (C1-C10)alkyl, or (C6-C18)aryl, for example, deuterium, methyl, or phenyl.

According to one embodiment, the organic electroluminescent compound represented by formula 2 above may be more specifically illustrated by the following compounds, but is not limited thereto.

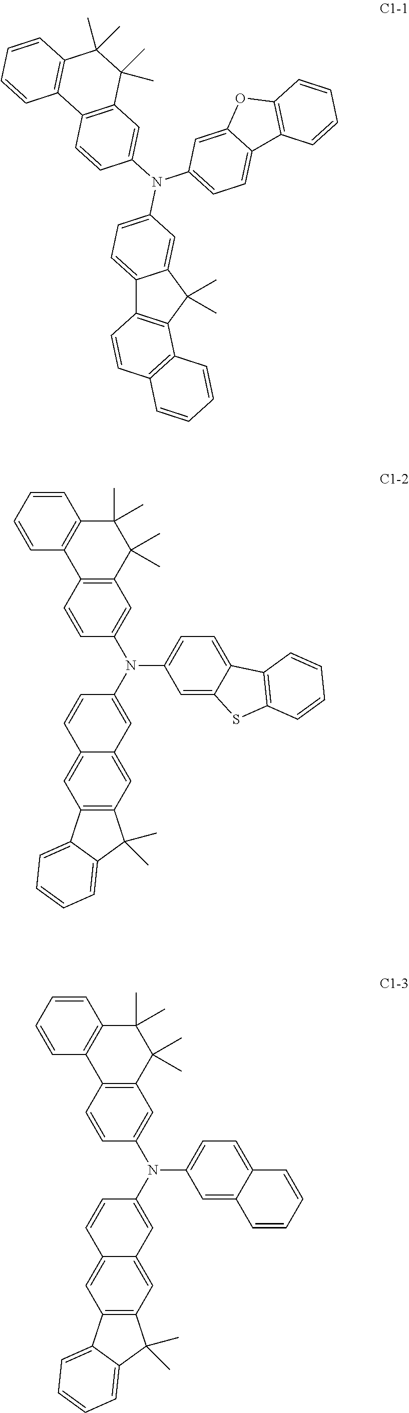 [SMILES: CC1(C)c2cc(N(c3ccc4c(c3)C(C)(C)C(C)(C)c3ccccc3-4)c3ccc4c(c3)oc3ccccc34)ccc2-c2ccc3ccccc3c21 ; CC1(C)c2ccccc2-c2cc3ccc(N(c4ccc5c(c4)C(C)(C)C(C)(C)c4ccccc4-5)c4ccc5c(c4)sc4ccccc45)cc3cc21 ; CC1(C)c2ccccc2-c2cc3ccc(N(c4ccc5c(c4)C(C)(C)C(C)(C)c4ccccc4-5)c4ccc5ccccc5c4)cc3cc21]

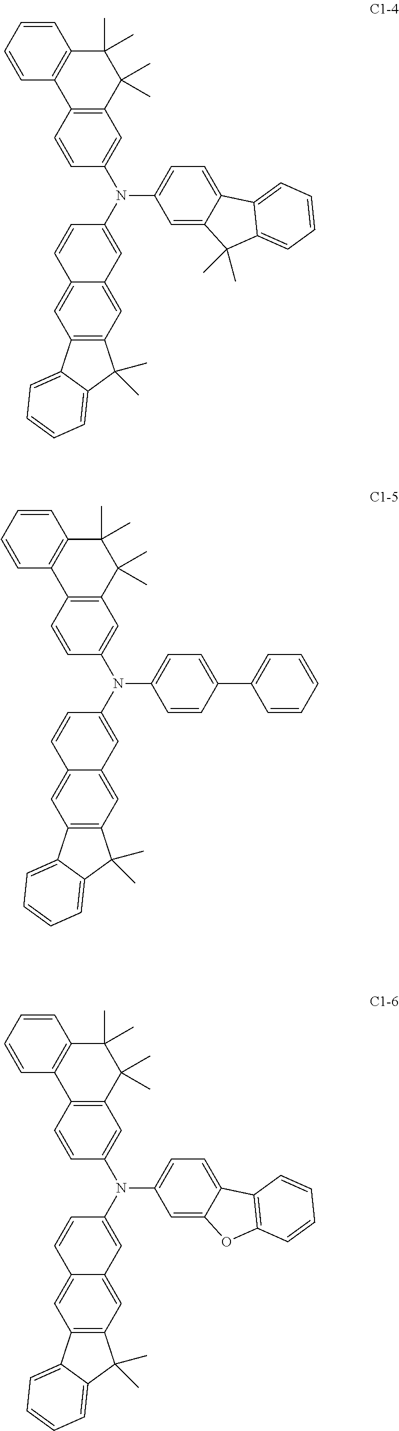 [SMILES: CC1(C)c2ccccc2-c2ccc(N(c3ccc4c(c3)C(C)(C)C(C)(C)c3ccccc3-4)c3ccc4cc5c(cc4c3)C(C)(C)c3ccccc3-5)cc21 ; CC1(C)c2ccccc2-c2cc3ccc(N(c4ccc(-c5ccccc5)cc4)c4ccc5c(c4)C(C)(C)C(C)(C)c4ccccc4-5)cc3cc21 ; CC1(C)c2ccccc2-c2cc3ccc(N(c4ccc5c(c4)C(C)(C)C(C)(C)c4ccccc4-5)c4ccc5c(c4)oc4ccccc45)cc3cc21]

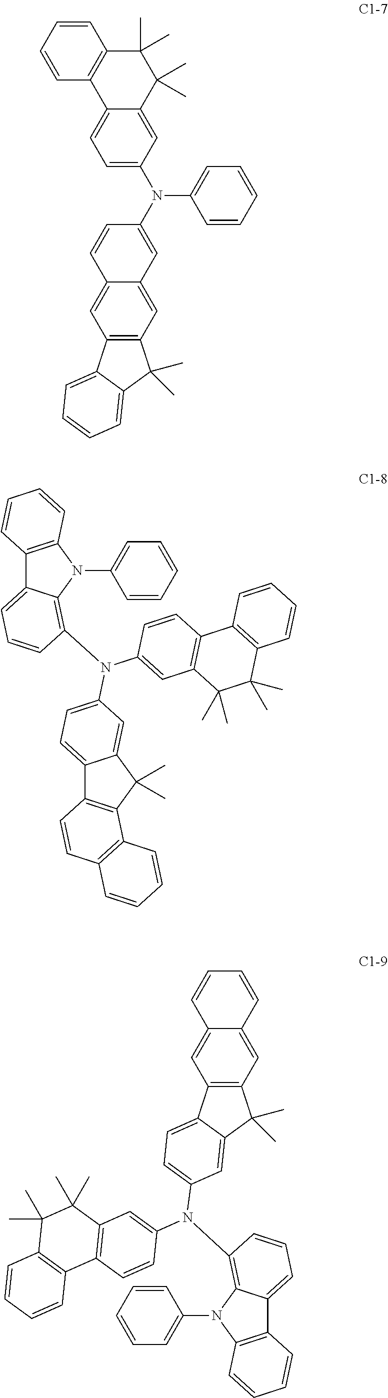 [SMILES: CC1(C)c2ccccc2-c2cc3ccc(N(c4ccccc4)c4ccc5c(c4)C(C)(C)C(C)(C)c4ccccc4-5)cc3cc21 ; CC1(C)c2cc(N(c3ccc4c(c3)C(C)(C)C(C)(C)c3ccccc3-4)c3cccc4c5ccccc5n(-c5ccccc5)c34)ccc2-c2ccc3ccccc3c21 ; CC1(C)c2cc(N(c3ccc4c(c3)C(C)(C)C(C)(C)c3ccccc3-4)c3cccc4c5ccccc5n(-c5ccccc5)c34)ccc2-c2cc3ccccc3cc21]

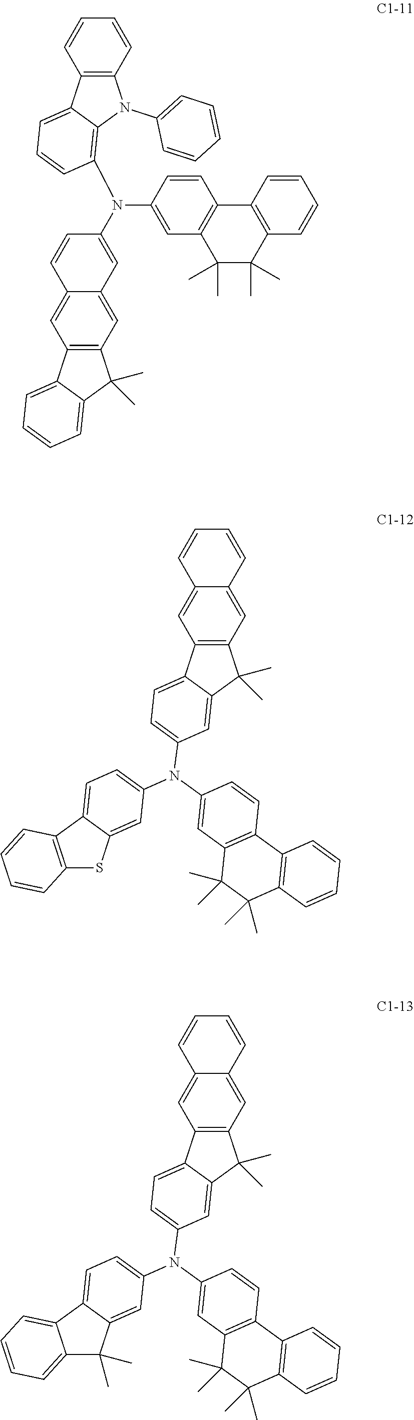 [SMILES: CC1(C)c2ccccc2-c2cc3ccc(N(c4ccc5c(c4)C(C)(C)C(C)(C)c4ccccc4-5)c4cccc5c6ccccc6n(-c6ccccc6)c45)cc3cc21 ; CC1(C)c2cc(N(c3ccc4c(c3)C(C)(C)C(C)(C)c3ccccc3-4)c3ccc4c(c3)sc3ccccc34)ccc2-c2cc3ccccc3cc21 ; CC1(C)c2ccccc2-c2ccc(N(c3ccc4c(c3)C(C)(C)c3cc5ccccc5cc3-4)c3ccc4c(c3)C(C)(C)C(C)(C)c3ccccc3-4)cc21]

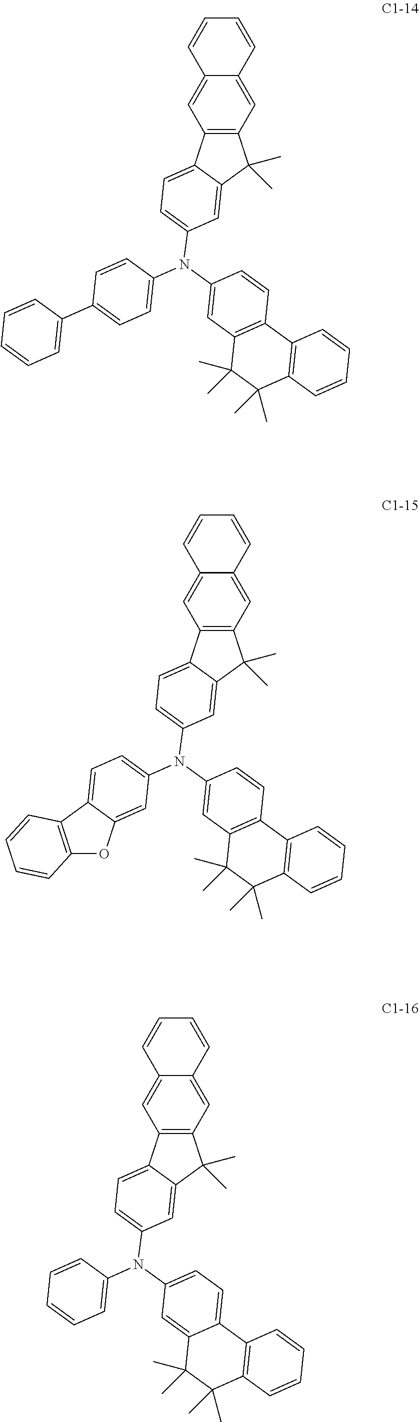 [SMILES: CC1(C)c2cc(N(c3ccc(-c4ccccc4)cc3)c3ccc4c(c3)C(C)(C)C(C)(C)c3ccccc3-4)ccc2-c2cc3ccccc3cc21 ; CC1(C)c2cc(N(c3ccc4c(c3)C(C)(C)C(C)(C)c3ccccc3-4)c3ccc4c(c3)oc3ccccc34)ccc2-c2cc3ccccc3cc21 ; CC1(C)c2cc(N(c3ccccc3)c3ccc4c(c3)C(C)(C)C(C)(C)c3ccccc3-4)ccc2-c2cc3ccccc3cc21]

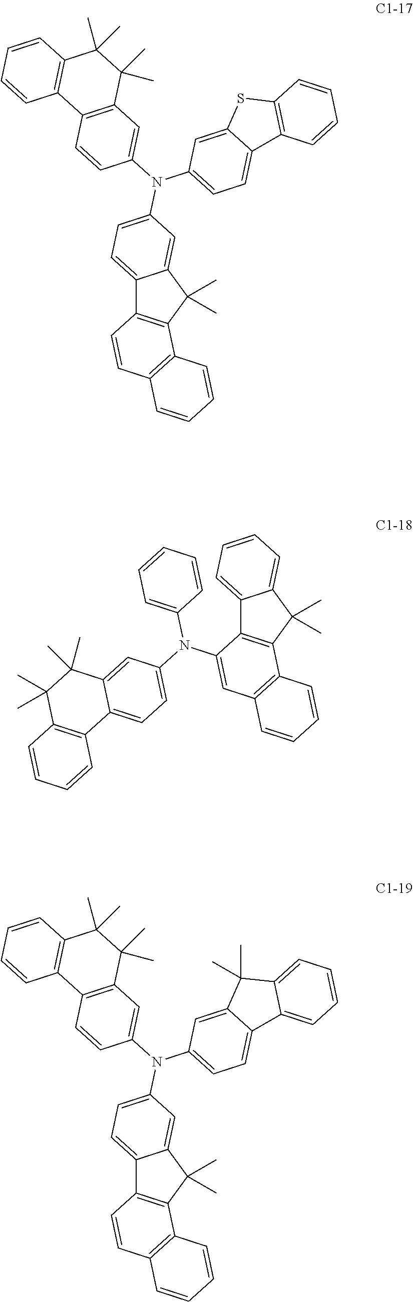

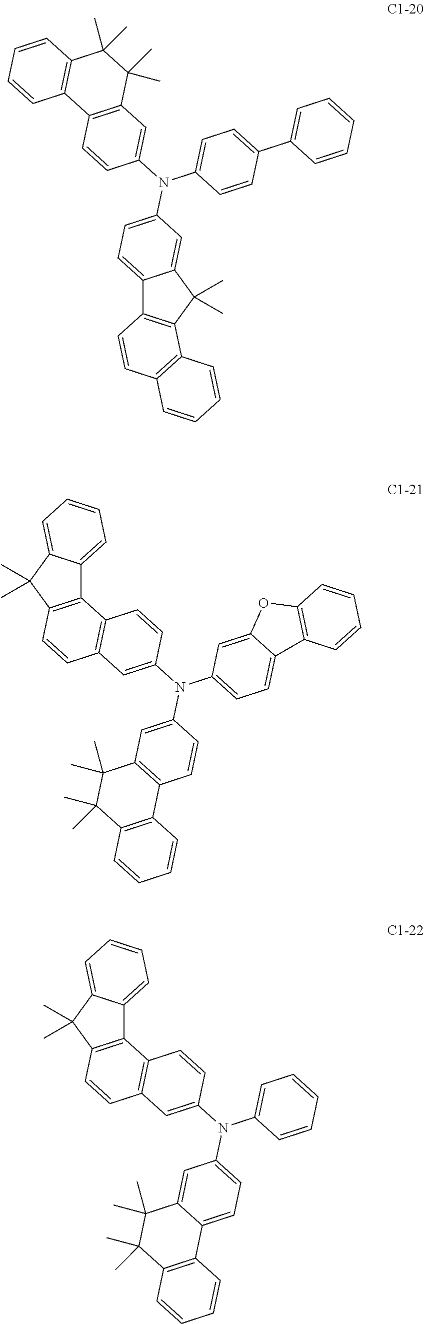 [SMILES: CC1(C)c2cc(N(c3ccc(-c4ccccc4)cc3)c3ccc4c(c3)C(C)(C)C(C)(C)c3ccccc3-4)ccc2-c2ccc3ccccc3c21 ; CC1(C)c2ccccc2-c2c1ccc1cc(N(c3ccc4c(c3)C(C)(C)C(C)(C)c3ccccc3-4)c3ccc4c(c3)oc3ccccc34)ccc21 ; CC1(C)c2ccccc2-c2c1ccc1cc(N(c3ccccc3)c3ccc4c(c3)C(C)(C)C(C)(C)c3ccccc3-4)ccc21]

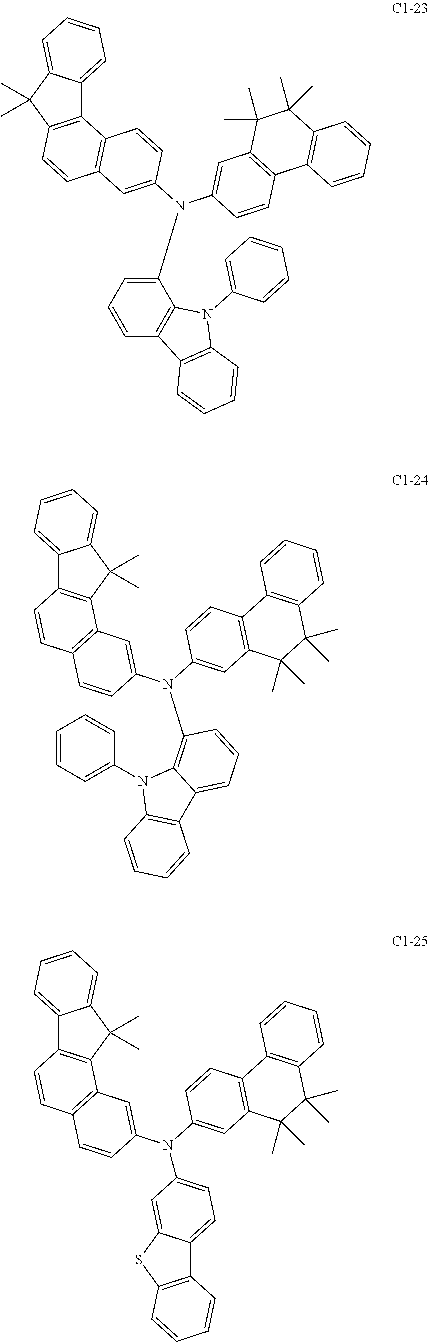 [SMILES: CC1(C)c2ccccc2-c2c1ccc1cc(N(c3ccc4c(c3)C(C)(C)C(C)(C)c3ccccc3-4)c3cccc4c5ccccc5n(-c5ccccc5)c34)ccc21 ; CC1(C)c2ccccc2-c2ccc3ccc(N(c4ccc5c(c4)C(C)(C)C(C)(C)c4ccccc4-5)c4cccc5c6ccccc6n(-c6ccccc6)c45)cc3c21 ; CC1(C)c2ccccc2-c2ccc3ccc(N(c4ccc5c(c4)C(C)(C)C(C)(C)c4ccccc4-5)c4ccc5c(c4)sc4ccccc45)cc3c21]

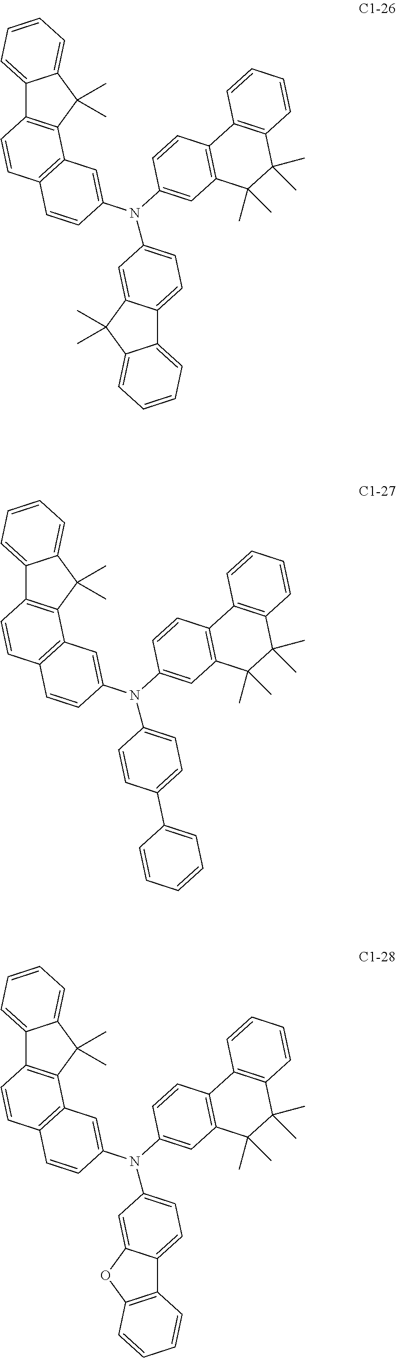 [SMILES: CC1(C)c2ccccc2-c2ccc(N(c3ccc4c(c3)C(C)(C)C(C)(C)c3ccccc3-4)c3ccc4ccc5c(c4c3)C(C)(C)c3ccccc3-5)cc21 ; CC1(C)c2ccccc2-c2ccc3ccc(N(c4ccc(-c5ccccc5)cc4)c4ccc5c(c4)C(C)(C)C(C)(C)c4ccccc4-5)cc3c21 ; CC1(C)c2ccccc2-c2ccc3ccc(N(c4ccc5c(c4)C(C)(C)C(C)(C)c4ccccc4-5)c4ccc5c(c4)oc4ccccc45)cc3c21]

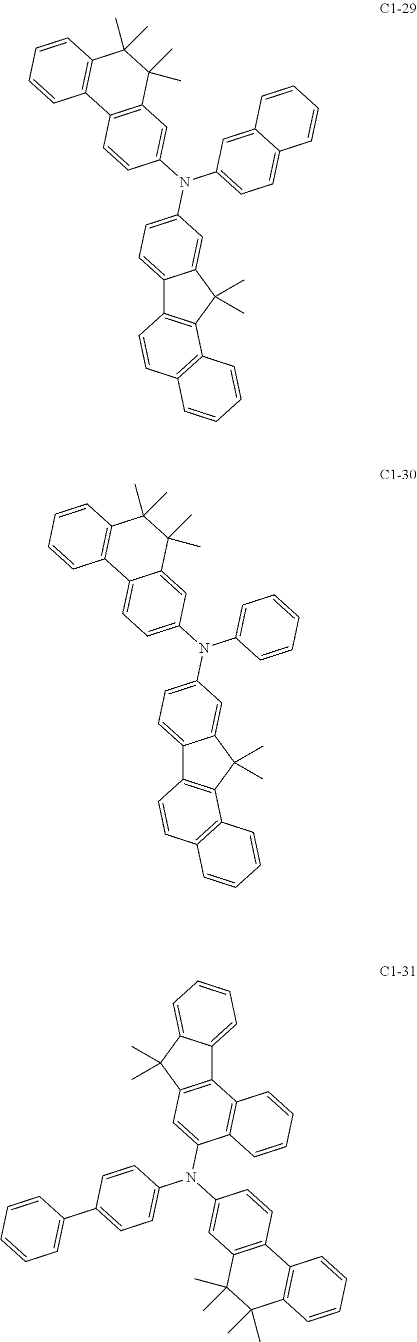 [SMILES: CC1(C)c2cc(N(c3ccc4c(c3)C(C)(C)C(C)(C)c3ccccc3-4)c3ccc4ccccc4c3)ccc2-c2ccc3ccccc3c21 ; CC1(C)c2cc(N(c3ccccc3)c3ccc4c(c3)C(C)(C)C(C)(C)c3ccccc3-4)ccc2-c2ccc3ccccc3c21 ; CC1(C)c2ccccc2-c2c1cc(N(c1ccc(-c3ccccc3)cc1)c1ccc3c(c1)C(C)(C)C(C)(C)c1ccccc1-3)c1ccccc21]

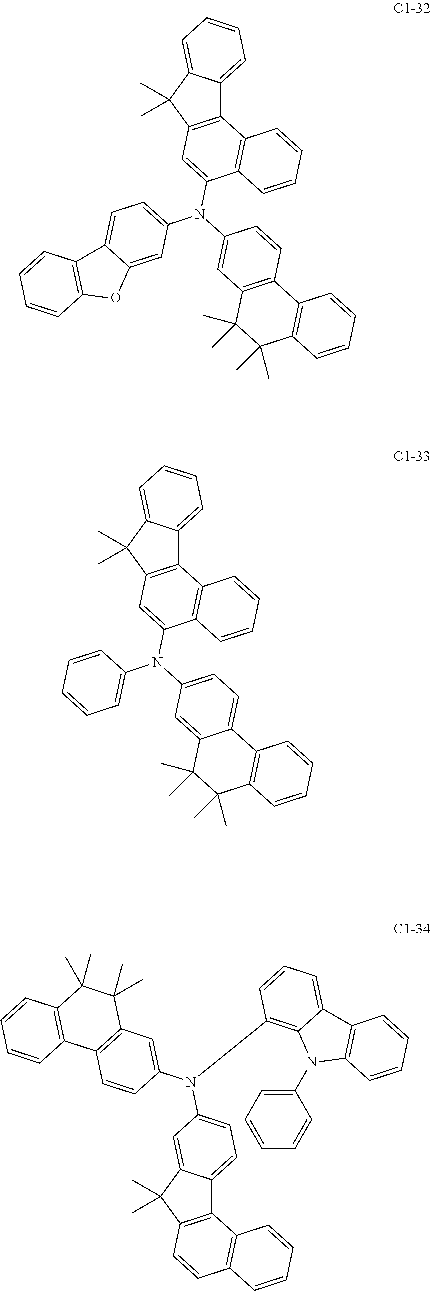 [SMILES: CC1(C)c2ccccc2-c2c1cc(N(c1ccc3c(c1)C(C)(C)C(C)(C)c1ccccc1-3)c1ccc3c(c1)oc1ccccc13)c1ccccc21 ; CC1(C)c2ccccc2-c2c1cc(N(c1ccccc1)c1ccc3c(c1)C(C)(C)C(C)(C)c1ccccc1-3)c1ccccc21 ; CC1(C)c2cc(N(c3ccc4c(c3)C(C)(C)C(C)(C)c3ccccc3-4)c3cccc4c5ccccc5n(-c5ccccc5)c34)ccc2-c2c1ccc1ccccc21]

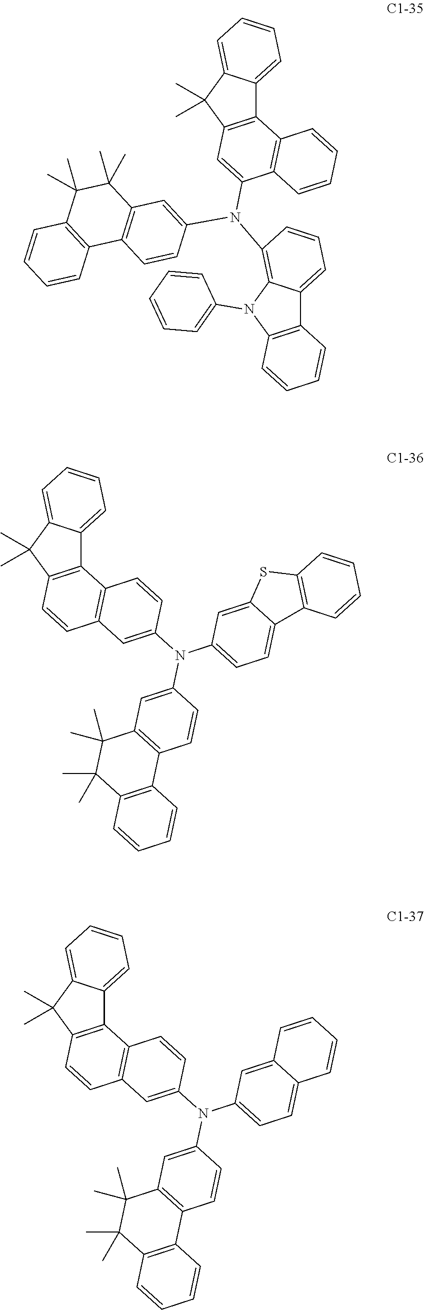 [SMILES: CC1(C)c2ccccc2-c2c1cc(N(c1ccc3c(c1)C(C)(C)C(C)(C)c1ccccc1-3)c1cccc3c4ccccc4n(-c4ccccc4)c13)c1ccccc21 ; CC1(C)c2ccccc2-c2c1ccc1cc(N(c3ccc4c(c3)C(C)(C)C(C)(C)c3ccccc3-4)c3ccc4c(c3)sc3ccccc34)ccc21 ; CC1(C)c2ccccc2-c2c1ccc1cc(N(c3ccc4c(c3)C(C)(C)C(C)(C)c3ccccc3-4)c3ccc4ccccc4c3)ccc21]

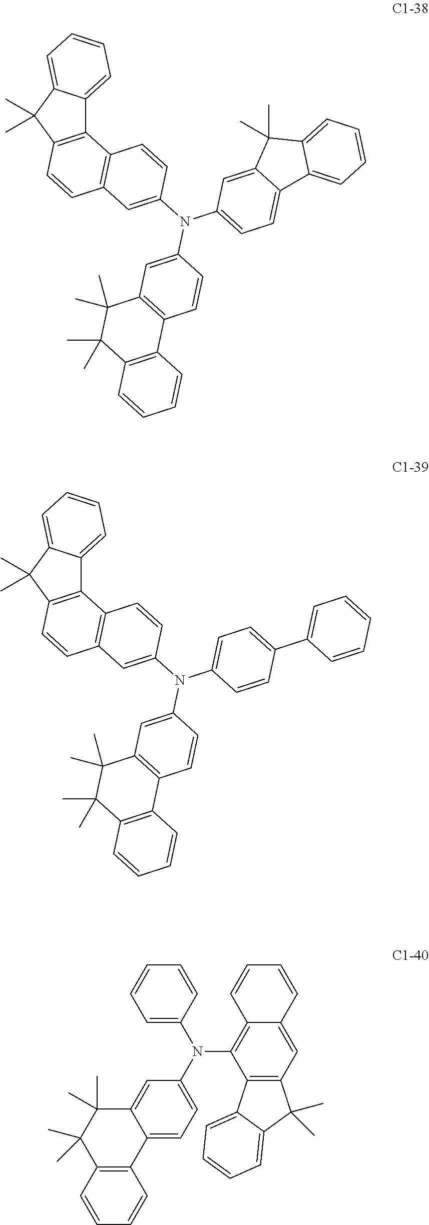

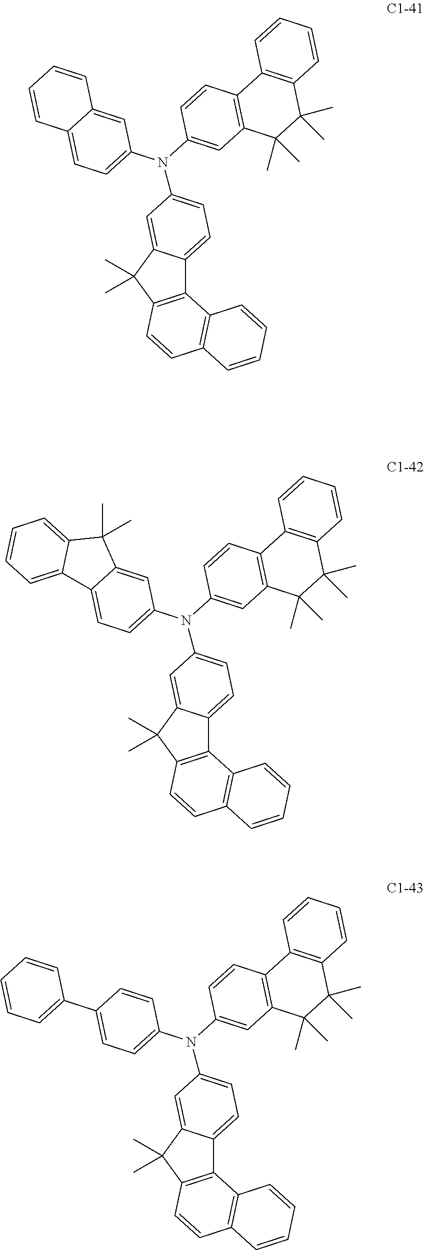 [SMILES: CC1(C)c2cc(N(c3ccc4c(c3)C(C)(C)C(C)(C)c3ccccc3-4)c3ccc4ccccc4c3)ccc2-c2c1ccc1ccccc21 ; CC1(C)c2ccccc2-c2ccc(N(c3ccc4c(c3)C(C)(C)c3ccc5ccccc5c3-4)c3ccc4c(c3)C(C)(C)C(C)(C)c3ccccc3-4)cc21 ; CC1(C)c2cc(N(c3ccc(-c4ccccc4)cc3)c3ccc4c(c3)C(C)(C)C(C)(C)c3ccccc3-4)ccc2-c2c1ccc1ccccc21]

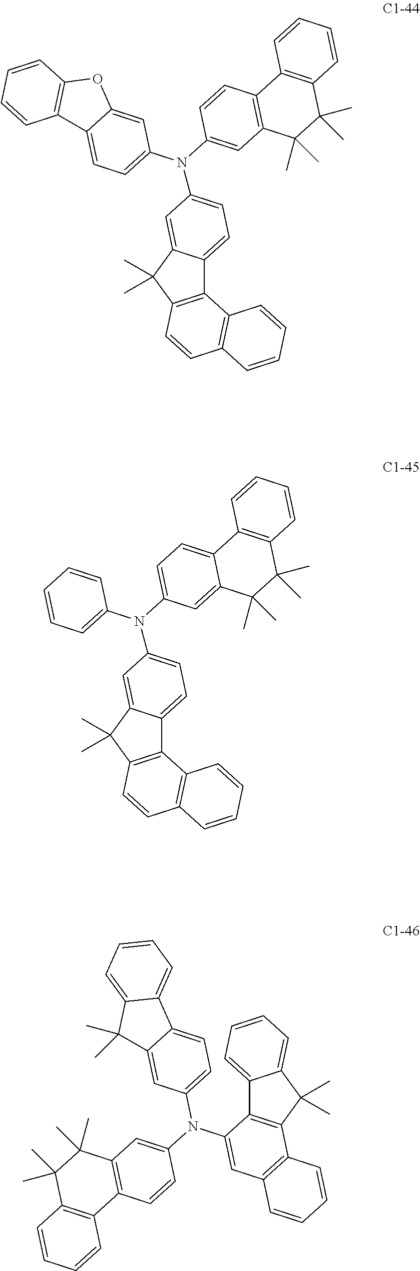

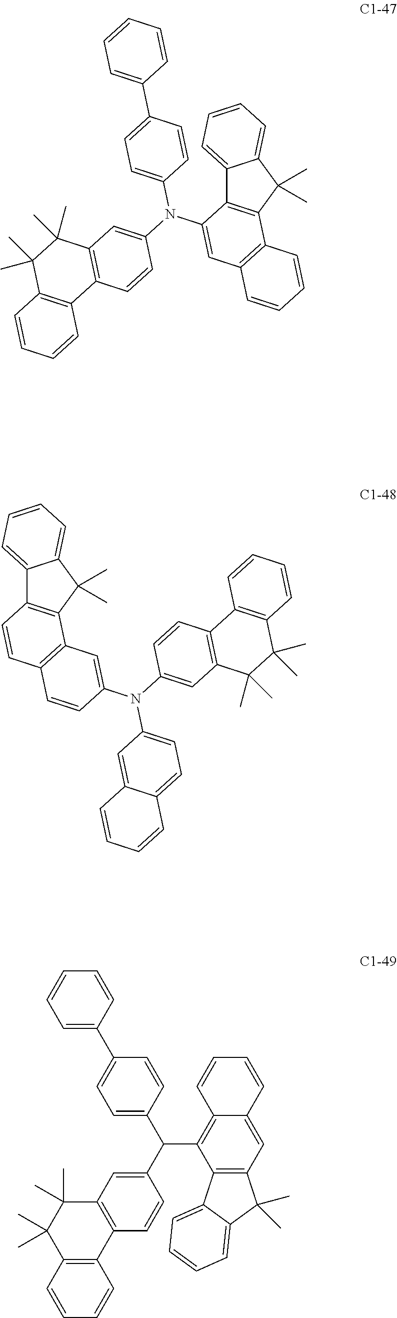

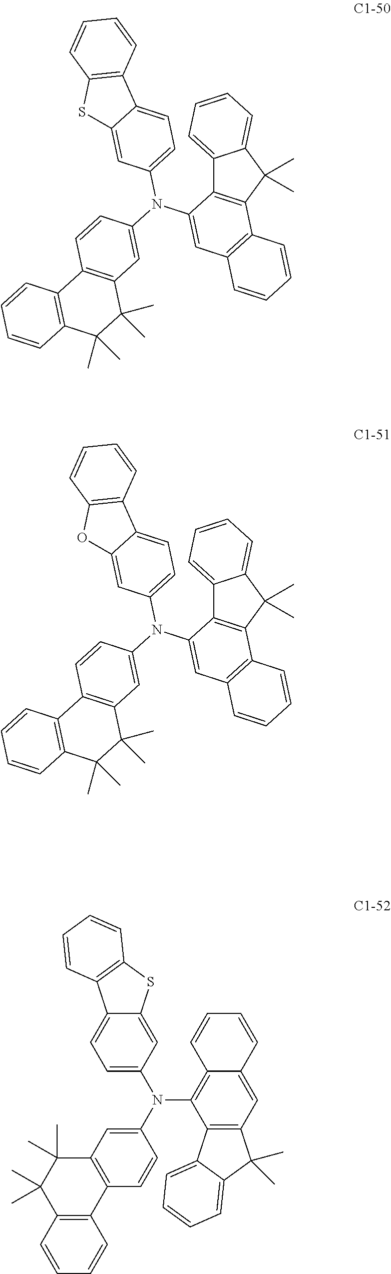

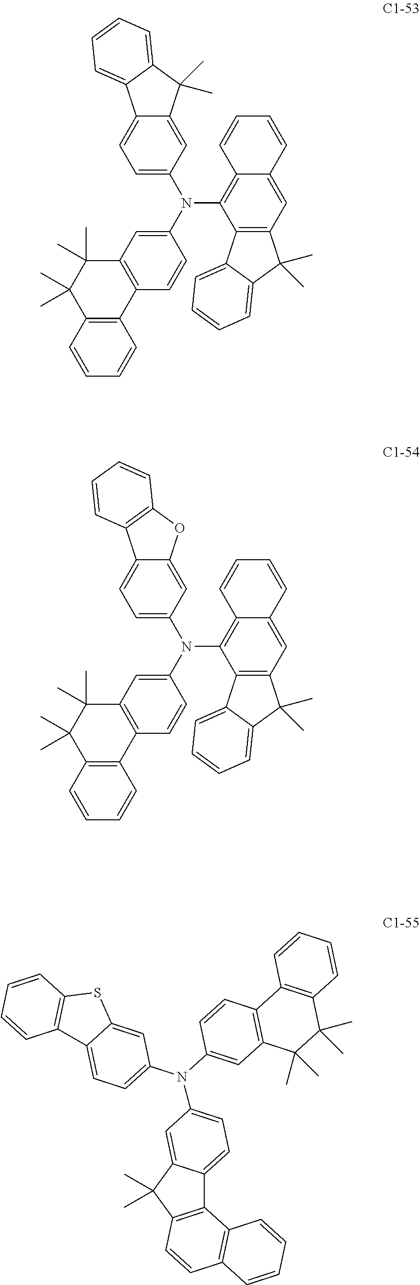

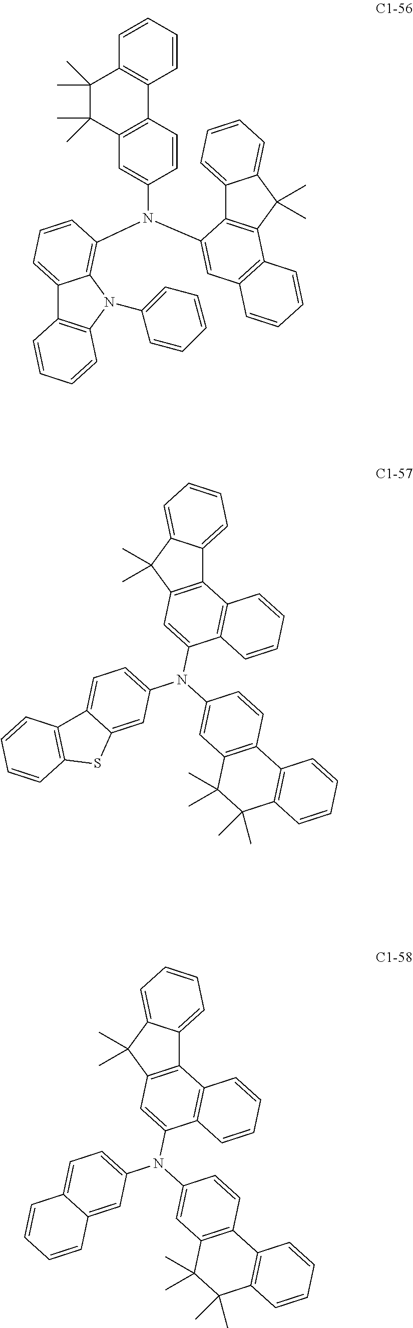 [SMILES: CC1(C)c2ccccc2-c2c(N(c3ccc4c(c3)C(C)(C)C(C)(C)c3ccccc3-4)c3cccc4c5ccccc5n(-c5ccccc5)c34)cc3ccccc3c21 ; CC1(C)c2ccccc2-c2c1cc(N(c1ccc3c(c1)C(C)(C)C(C)(C)c1ccccc1-3)c1ccc3c(c1)sc1ccccc13)c1ccccc21 ; CC1(C)c2ccccc2-c2c1cc(N(c1ccc3c(c1)C(C)(C)C(C)(C)c1ccccc1-3)c1ccc3ccccc3c1)c1ccccc21]

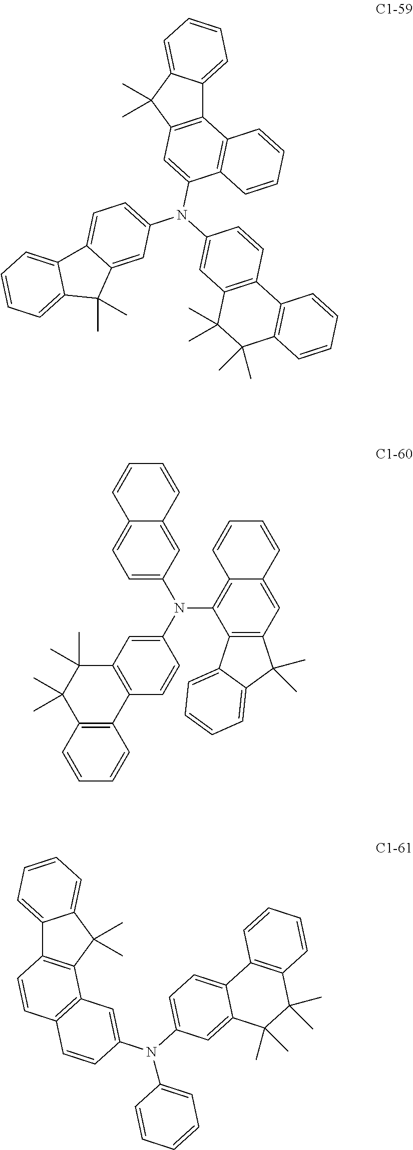

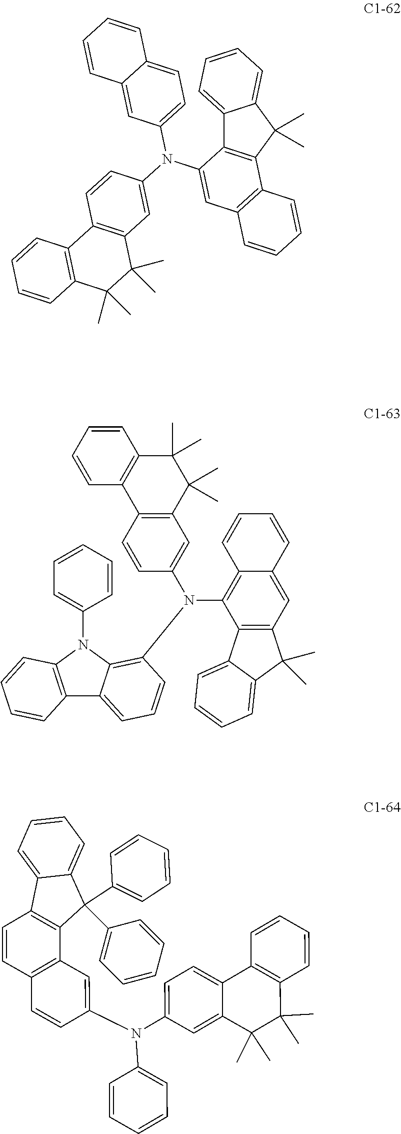 [SMILES: CC1(C)c2ccccc2-c2c(N(c3ccc4c(c3)C(C)(C)C(C)(C)c3ccccc3-4)c3ccc4ccccc4c3)cc3ccccc3c21 ; CC1(C)c2ccccc2-c2c1cc1ccccc1c2N(c1ccc2c(c1)C(C)(C)C(C)(C)c1ccccc1-2)c1cccc2c3ccccc3n(-c3ccccc3)c12 ; CC1(C)c2ccccc2-c2ccc(N(c3ccccc3)c3ccc4ccc5c(c4c3)C(c3ccccc3)(c3ccccc3)c3ccccc3-5)cc2C1(C)C]

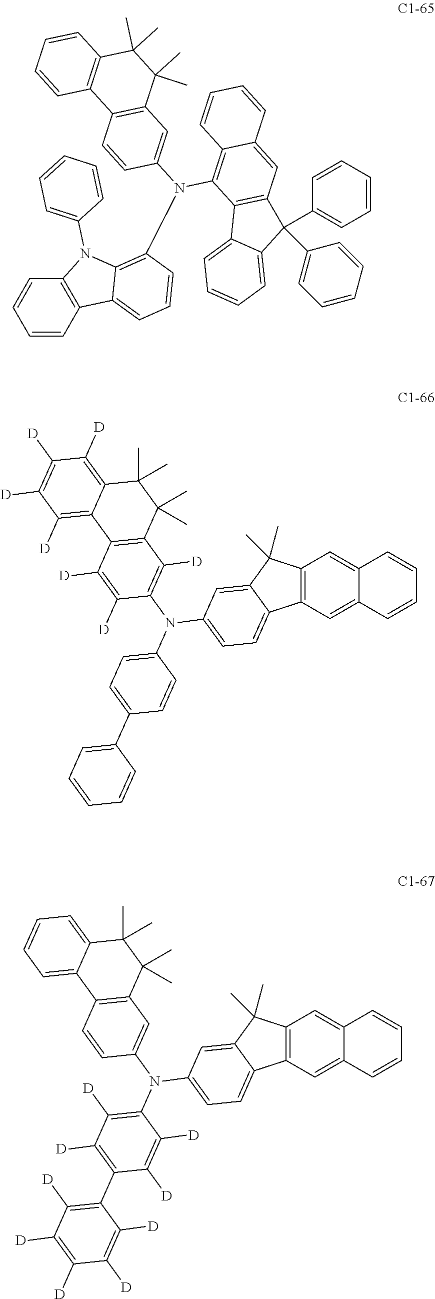 [SMILES: CC1(C)c2ccccc2-c2ccc(N(c3c4c(cc5ccccc35)C(c3ccccc3)(c3ccccc3)c3ccccc3-4)c3cccc4c5ccccc5n(-c5ccccc5)c34)cc2C1(C)C ; [2H]c1c([2H])c([2H])c2c(c1[2H])-c1c([2H])c([2H])c(N(c3ccc(-c4ccccc4)cc3)c3ccc4c(c3)C(C)(C)c3cc5ccccc5cc3-4)c([2H])c1C(C)(C)C2(C)C ; [2H]c1c([2H])c([2H])c(-c2c([2H])c([2H])c(N(c3ccc4c(c3)C(C)(C)c3cc5ccccc5cc3-4)c3ccc4c(c3)C(C)(C)C(C)(C)c3ccccc3-4)c([2H])c2[2H])c([2H])c1[2H]]

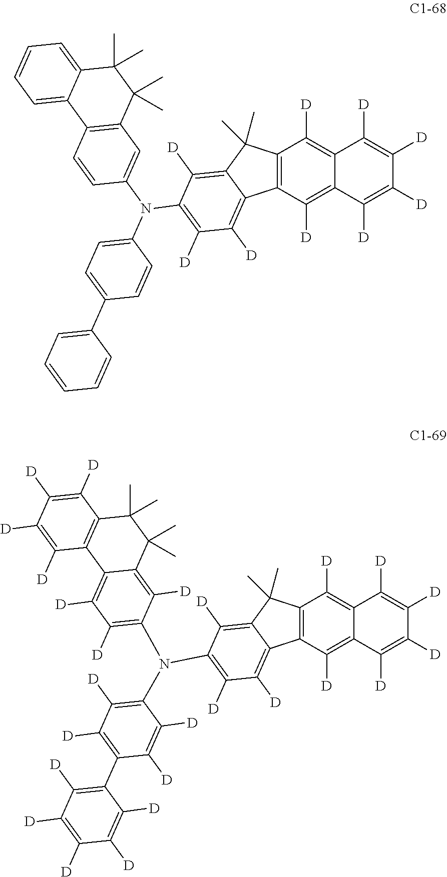 [SMILES: [2H]c1c([2H])c(N(c2ccc(-c3ccccc3)cc2)c2ccc3c(c2)C(C)(C)C(C)(C)c2ccccc2-3)c([2H])c2c1-c1c(c([2H])c3c([2H])c([2H])c([2H])c([2H])c3c1[2H])C2(C)C ; [2H]c1c([2H])c([2H])c(-c2c([2H])c([2H])c(N(c3c([2H])c([2H])c4c(c3[2H])C(C)(C)c3c-4c([2H])c4c([2H])c([2H])c([2H])c([2H])c4c3[2H])c3c([2H])c([2H])c4c(c3[2H])C(C)(C)C(C)(C)c3c([2H])c([2H])c([2H])c([2H])c3-4)c([2H])c2[2H])c([2H])c1[2H]]

The organic electroluminescent compound of formula 2 according to the present disclosure may be produced by referring to the reactions as represented by reaction schemes 1 to 3 above, but is not limited thereto. Further, it may be prepared by a synthetic method known to a person skilled in the art.

The organic electroluminescent compound according to other embodiment may be represented by the following formula 3.

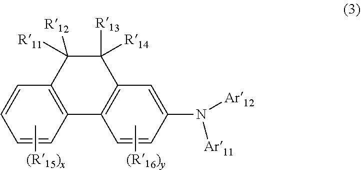

In formula 3.

R′₁₁ to R′₁₄ each independently represent substituted or unsubstituted methyl;

R′₁₅ and R′₁₆ each independently represent hydrogen, or deuterium;

Ar′₁₁ and Ar′₁₂ each independently represent phenyl unsubstituted or substituted with deuterium, biphenyl unsubstituted or substituted with deuterium, terphenyl unsubstituted or substituted with deuterium, naphthyl unsubstituted or substituted with deuterium, a group of the following formula (a) unsubstituted or substituted with deuterium, or a combination thereof:

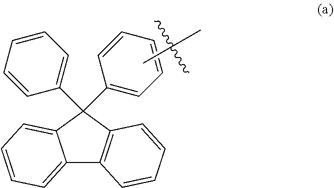

x represents an integer of 1 to 4, y represents an integer of 1 to 3, and when x and y are 2 or more, each of R′₁₅ and each of R′₁₆ may be the same or different.

In one embodiment, all of R′₁₁ to R′₁₄ may be unsubstituted methyl.

In one embodiment, all of R′₁₅ and R′₁₆ may be hydrogen, or all of R′₁₅ and R′₁₆ may be deuterium.

In one embodiment, Ar′₁₁ and Ar′₁₂ each independently may be phenyl unsubstituted or substituted with deuterium, biphenyl unsubstituted or substituted with deuterium, terphenyl unsubstituted or substituted with deuterium, naphthyl unsubstituted or substituted with deuterium, a group of formula (a) above unsubstituted or substituted with deuterium, or a combination thereof, preferably, unsubstituted phenyl, unsubstituted o-biphenyl, unsubstituted m-biphenyl, p-biphenyl unsubstituted or substituted with deuterium, unsubstituted o-terphenyl, unsubstituted m-terphenyl, unsubstituted p-biphenyl, or unsubstituted group of formula (a) above, or a combination thereof.

According to one embodiment, the organic electroluminescent compound represented by formula 3 above may be more specifically illustrated by the following compounds, but is not limited thereto.

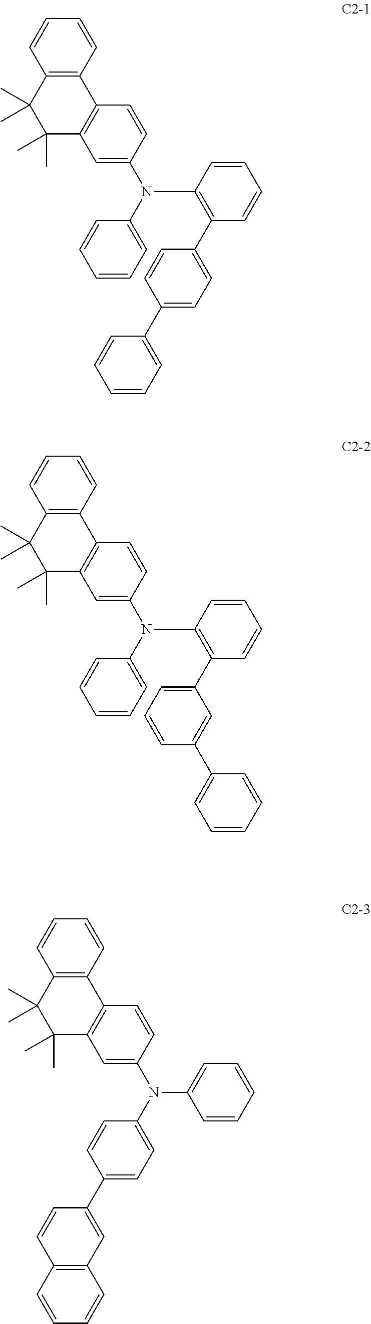 [SMILES: CC1(C)c2ccccc2-c2ccc(N(c3ccccc3)c3ccccc3-c3ccc(-c4ccccc4)cc3)cc2C1(C)C ; CC1(C)c2ccccc2-c2ccc(N(c3ccccc3)c3ccccc3-c3cccc(-c4ccccc4)c3)cc2C1(C)C ; CC1(C)c2ccccc2-c2ccc(N(c3ccccc3)c3ccc(-c4ccc5ccccc5c4)cc3)cc2C1(C)C]

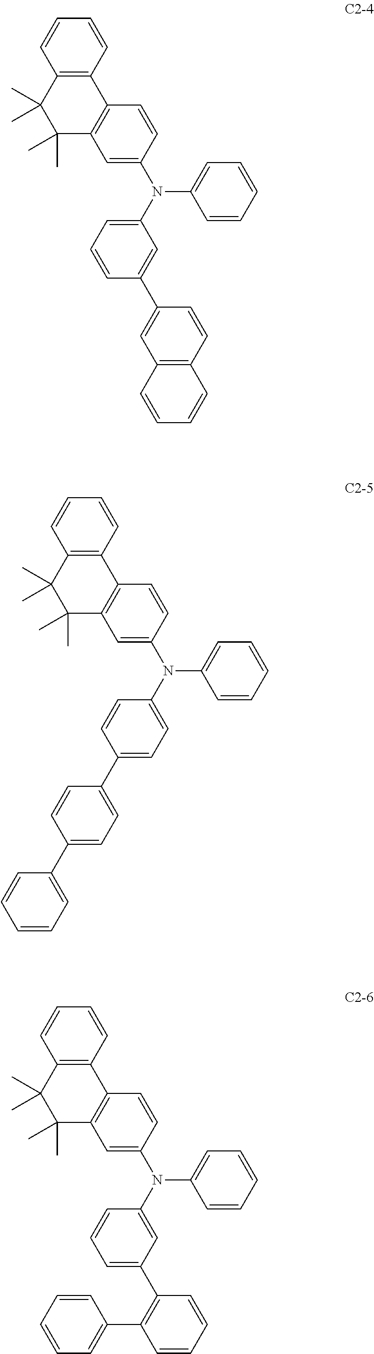 [SMILES: CC1(C)c2ccccc2-c2ccc(N(c3ccccc3)c3cccc(-c4ccc5ccccc5c4)c3)cc2C1(C)C ; CC1(C)c2ccccc2-c2ccc(N(c3ccccc3)c3ccc(-c4ccc(-c5ccccc5)cc4)cc3)cc2C1(C)C ; CC1(C)c2ccccc2-c2ccc(N(c3ccccc3)c3cccc(-c4ccccc4-c4ccccc4)c3)cc2C1(C)C]

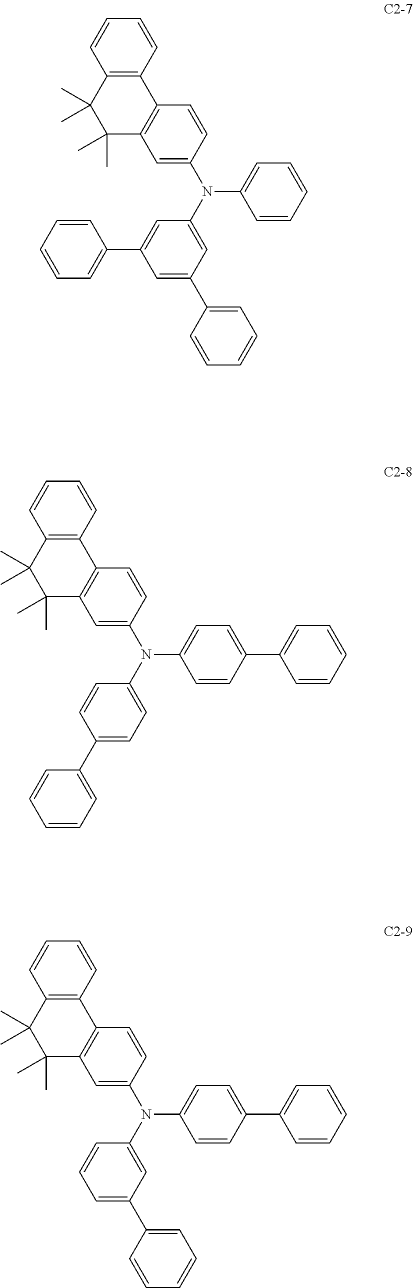 [SMILES: CC1(C)c2ccccc2-c2ccc(N(c3ccccc3)c3cc(-c4ccccc4)cc(-c4ccccc4)c3)cc2C1(C)C ; CC1(C)c2ccccc2-c2ccc(N(c3ccc(-c4ccccc4)cc3)c3ccc(-c4ccccc4)cc3)cc2C1(C)C ; CC1(C)c2ccccc2-c2ccc(N(c3ccc(-c4ccccc4)cc3)c3cccc(-c4ccccc4)c3)cc2C1(C)C]

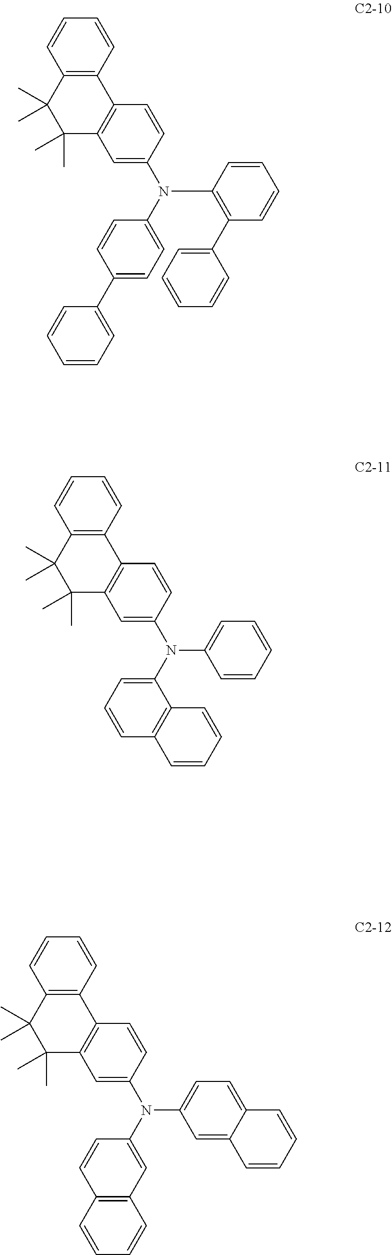 [SMILES: CC1(C)c2ccccc2-c2ccc(N(c3ccc(-c4ccccc4)cc3)c3ccccc3-c3ccccc3)cc2C1(C)C ; CC1(C)c2ccccc2-c2ccc(N(c3ccccc3)c3cccc4ccccc34)cc2C1(C)C ; CC1(C)c2ccccc2-c2ccc(N(c3ccc4ccccc4c3)c3ccc4ccccc4c3)cc2C1(C)C]

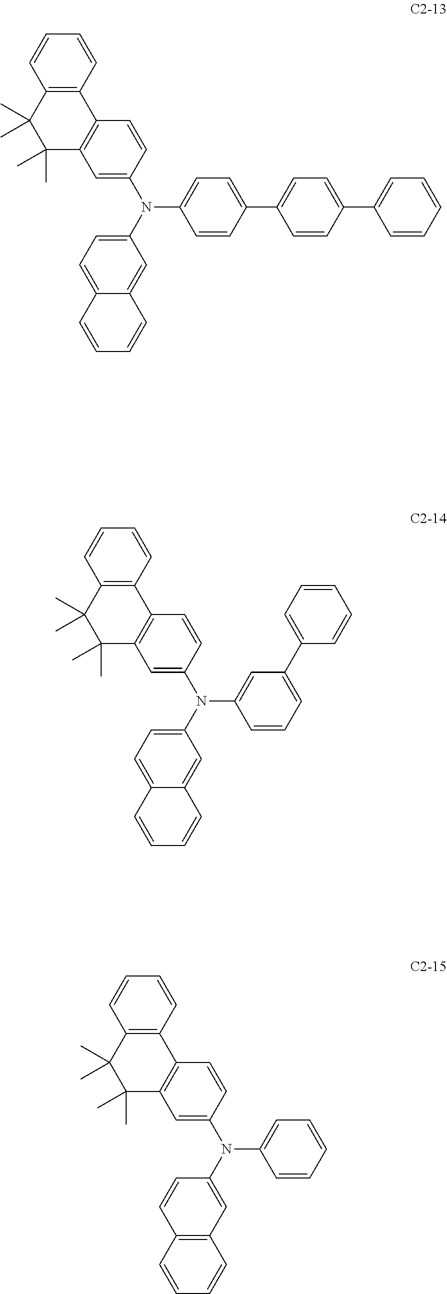 [SMILES: CC1(C)c2ccccc2-c2ccc(N(c3ccc(-c4ccc(-c5ccccc5)cc4)cc3)c3ccc4ccccc4c3)cc2C1(C)C ; CC1(C)c2ccccc2-c2ccc(N(c3cccc(-c4ccccc4)c3)c3ccc4ccccc4c3)cc2C1(C)C ; CC1(C)c2ccccc2-c2ccc(N(c3ccccc3)c3ccc4ccccc4c3)cc2C1(C)C]

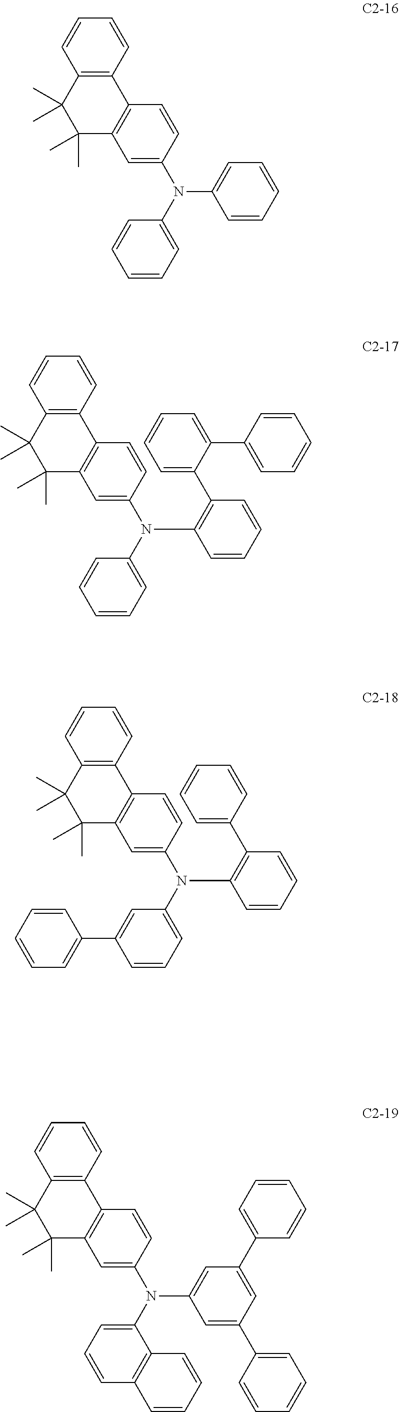 [SMILES: CC1(C)c2ccccc2-c2ccc(N(c3ccccc3)c3ccccc3)cc2C1(C)C ; CC1(C)c2ccccc2-c2ccc(N(c3ccccc3)c3ccccc3-c3ccccc3-c3ccccc3)cc2C1(C)C ; CC1(C)c2ccccc2-c2ccc(N(c3cccc(-c4ccccc4)c3)c3ccccc3-c3ccccc3)cc2C1(C)C ; CC1(C)c2ccccc2-c2ccc(N(c3cc(-c4ccccc4)cc(-c4ccccc4)c3)c3cccc4ccccc34)cc2C1(C)C]

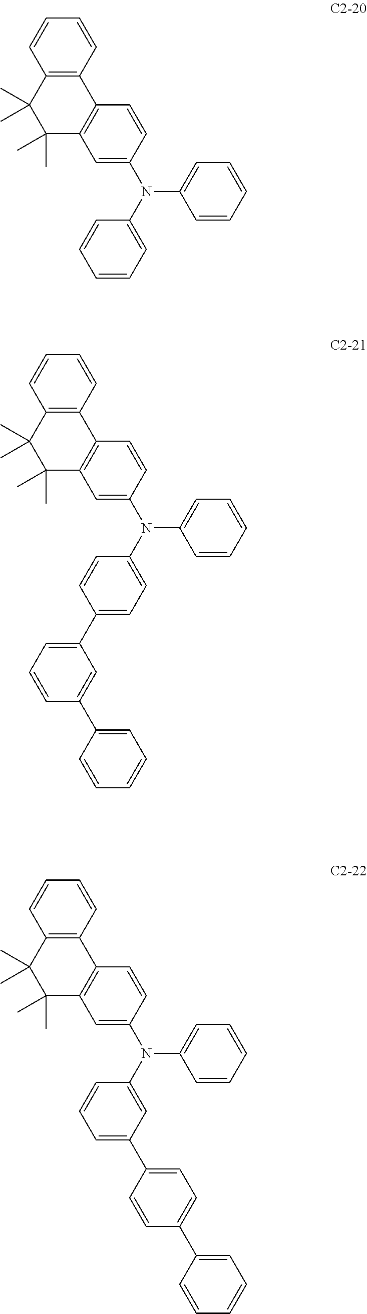 [SMILES: CC1(C)c2ccccc2-c2ccc(N(c3ccccc3)c3ccccc3)cc2C1(C)C ; CC1(C)c2ccccc2-c2ccc(N(c3ccccc3)c3ccc(-c4cccc(-c5ccccc5)c4)cc3)cc2C1(C)C ; CC1(C)c2ccccc2-c2ccc(N(c3ccccc3)c3cccc(-c4ccc(-c5ccccc5)cc4)c3)cc2C1(C)C]

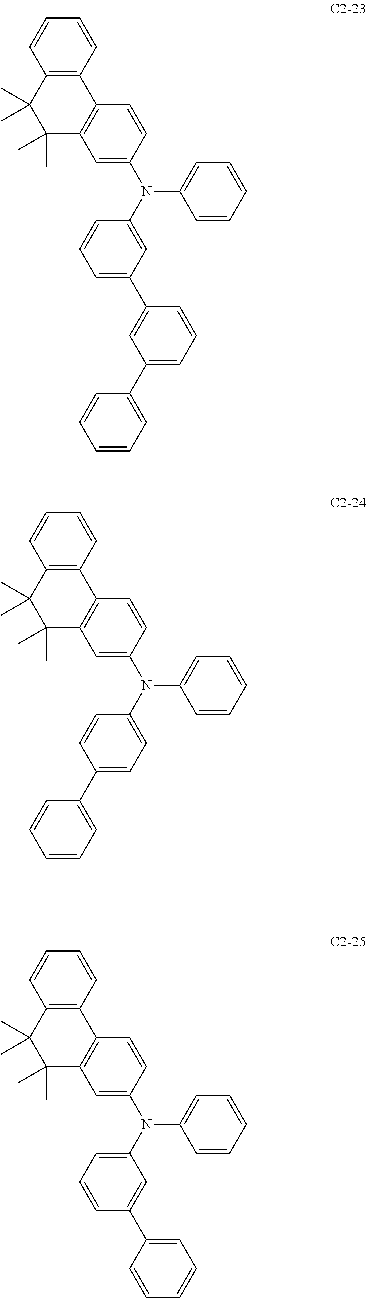 [SMILES: CC1(C)c2ccccc2-c2ccc(N(c3ccccc3)c3cccc(-c4cccc(-c5ccccc5)c4)c3)cc2C1(C)C ; CC1(C)c2ccccc2-c2ccc(N(c3ccccc3)c3ccc(-c4ccccc4)cc3)cc2C1(C)C ; CC1(C)c2ccccc2-c2ccc(N(c3ccccc3)c3cccc(-c4ccccc4)c3)cc2C1(C)C]

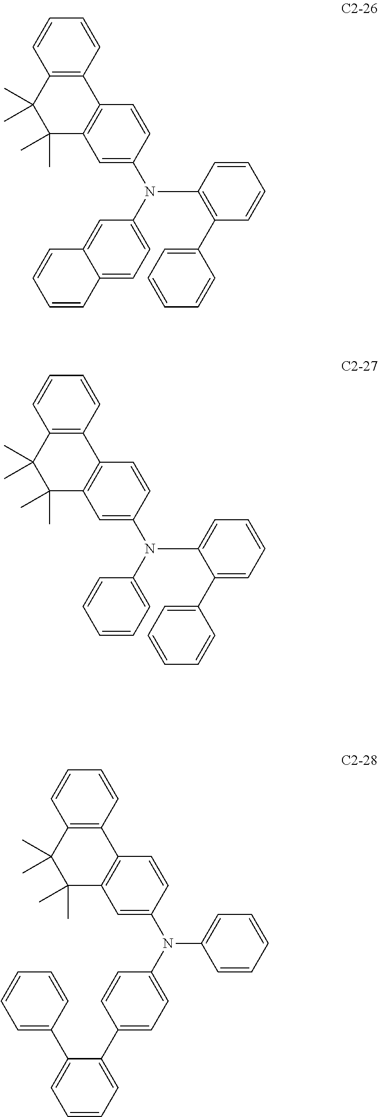 [SMILES: CC1(C)c2ccccc2-c2ccc(N(c3ccc4ccccc4c3)c3ccccc3-c3ccccc3)cc2C1(C)C ; CC1(C)c2ccccc2-c2ccc(N(c3ccccc3)c3ccccc3-c3ccccc3)cc2C1(C)C ; CC1(C)c2ccccc2-c2ccc(N(c3ccccc3)c3ccc(-c4ccccc4-c4ccccc4)cc3)cc2C1(C)C]

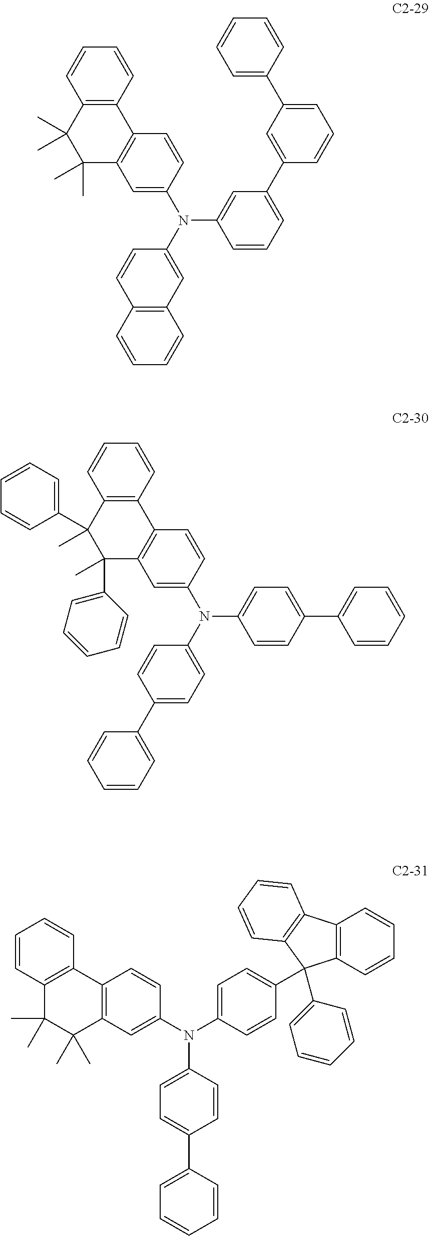 [SMILES: CC1(C)c2ccccc2-c2ccc(N(c3cccc(-c4cccc(-c5ccccc5)c4)c3)c3ccc4ccccc4c3)cc2C1(C)C ; CC1(c2ccccc2)c2ccccc2-c2ccc(N(c3ccc(-c4ccccc4)cc3)c3ccc(-c4ccccc4)cc3)cc2C1(C)c1ccccc1 ; CC1(C)c2ccccc2-c2ccc(N(c3ccc(-c4ccccc4)cc3)c3ccc(C4(c5ccccc5)c5ccccc5-c5ccccc54)cc3)cc2C1(C)C]

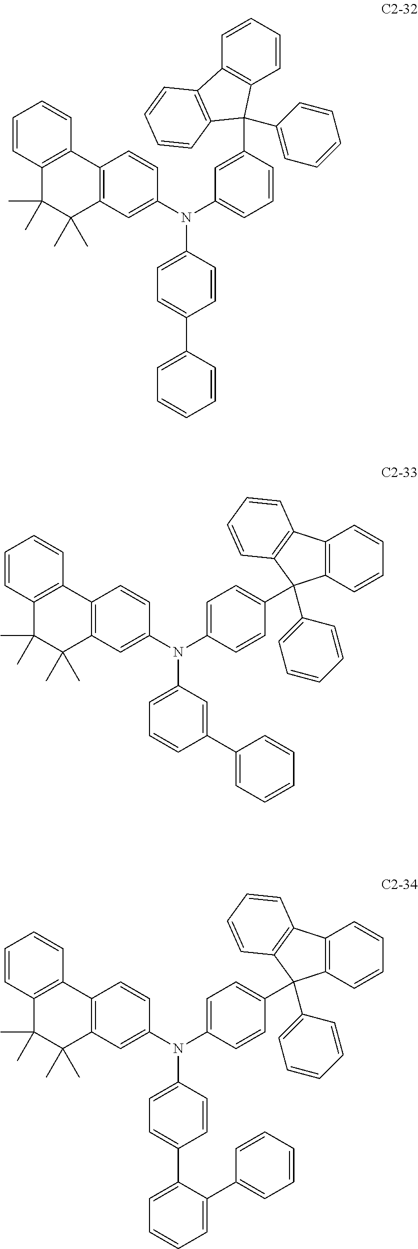 [SMILES: CC1(C)c2ccccc2-c2ccc(N(c3ccc(-c4ccccc4)cc3)c3cccc(C4(c5ccccc5)c5ccccc5-c5ccccc54)c3)cc2C1(C)C ; CC1(C)c2ccccc2-c2ccc(N(c3ccc(C4(c5ccccc5)c5ccccc5-c5ccccc54)cc3)c3cccc(-c4ccccc4)c3)cc2C1(C)C ; CC1(C)c2ccccc2-c2ccc(N(c3ccc(-c4ccccc4-c4ccccc4)cc3)c3ccc(C4(c5ccccc5)c5ccccc5-c5ccccc54)cc3)cc2C1(C)C]

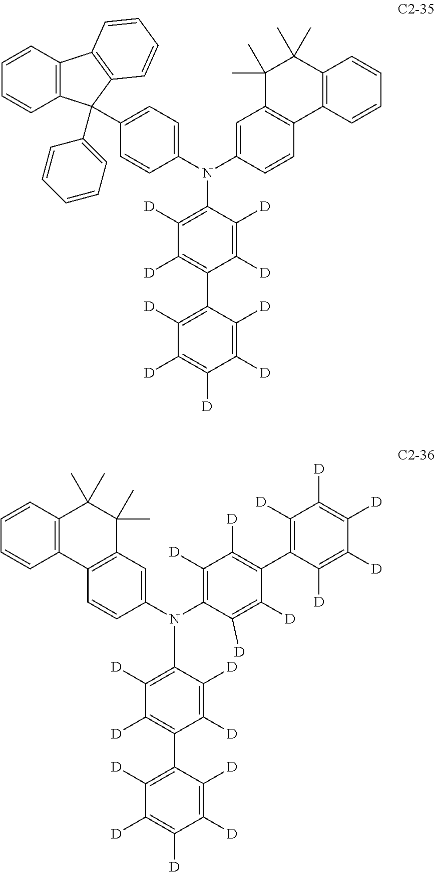 [SMILES: [2H]c1c([2H])c([2H])c(-c2c([2H])c([2H])c(N(c3ccc(C4(c5ccccc5)c5ccccc5-c5ccccc54)cc3)c3ccc4c(c3)C(C)(C)C(C)(C)c3ccccc3-4)c([2H])c2[2H])c([2H])c1[2H] ; [2H]c1c([2H])c([2H])c(-c2c([2H])c([2H])c(N(c3ccc4c(c3)C(C)(C)C(C)(C)c3ccccc3-4)c3c([2H])c([2H])c(-c4c([2H])c([2H])c([2H])c([2H])c4[2H])c([2H])c3[2H])c([2H])c2[2H])c([2H])c1[2H]]

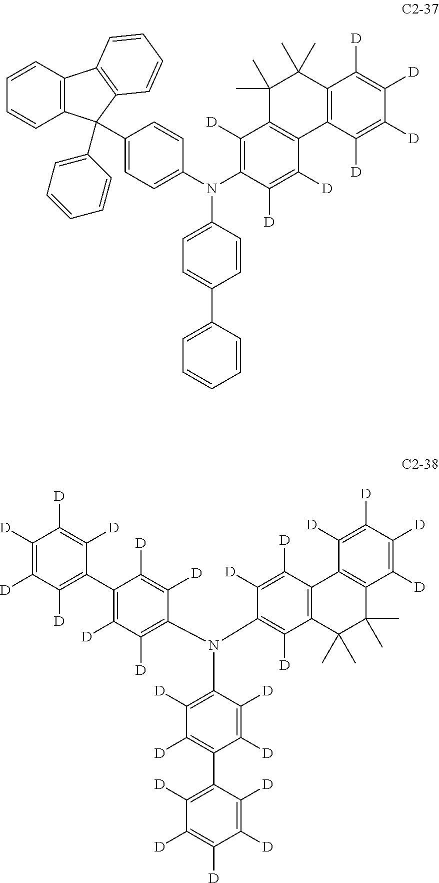 [SMILES: [2H]c1c([2H])c([2H])c2c(c1[2H])-c1c([2H])c([2H])c(N(c3ccc(-c4ccccc4)cc3)c3ccc(C4(c5ccccc5)c5ccccc5-c5ccccc54)cc3)c([2H])c1C(C)(C)C2(C)C ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])C(C)(C)C(C)(C)c3c([2H])c([2H])c([2H])c([2H])c3-4)c([2H])c2[2H])c([2H])c1[2H]]

The organic electroluminescent compound of formula 3 according to the present disclosure may be produced by referring to the reactions as represented by reaction schemes 1 to 3 above, but is not limited thereto. Further, it may be prepared by a synthetic method known to a person skilled in the art.

The present disclosure can provide an organic electroluminescent material comprising an organic electroluminescent compound of formula 1, and an organic electroluminescent device comprising the organic electroluminescent material.

Further, the present disclosure can provide an organic electroluminescent compound of formula 2, and an organic electroluminescent device comprising the same.

Furthermore, the present disclosure can provide an organic electroluminescent compound of formula 3, and an organic electroluminescent device comprising the same.

According to one embodiment of the present disclosure, the organic electroluminescent material of the present disclosure may be comprised solely of the organic electroluminescent compound of formula 1, or may further comprise conventional materials included in the organic electroluminescent material. In one embodiment, the compound of formula 1 above may be included as a hole transport material in the hole transport zone. The hole transport zone may be composed of one or more layers from the group consisting of a hole transport layer, a hole injection layer, an electron blocking layer, and a hole auxiliary layer, and each of the layers may be composed of one or more layers. In another embodiment, the compound of formula 1 above may be included as an electron transport material in the electron transport zone. The electron transport zone may be composed of one or more layers from the group consisting of an electron transport layer, an electron injection layer, a hole blocking layer, and an electron auxiliary layer, and each of the layers may be composed of one or more layers. In another embodiment, the compound of formula 1 above may be included as a host material in the light-emitting layer.

According to another embodiment of the present disclosure, the organic electroluminescent compound represented by formula 2 and/or the organic electroluminescent compound represented by formula 3 may be included as a hole transport material in the hole transport zone.

The organic electroluminescent material of the present disclosure may further include at least one host compounds and at least one dopant, in addition to the organic electroluminescent compound of formula 1 above.

The host material comprised in the organic electroluminescent material of the present disclosure may further comprise an organic electroluminescent compound which is different from the organic electroluminescent compound of formula 1 (a first host material), as a second host material. That is, the organic electroluminescent material according to one embodiment of the present disclosure may comprise a plurality of host materials. Specifically, the plurality of host materials according to one embodiment may comprise at least one compound of formula 1 as a first host material and at least one second host material which is different from the first host material. The weight ratio between the first host material and the second host material is in a ratio of 1:99 to 99:1, preferably, 10:90 to 90:10, and more preferably, 30:70 to 70:30.

The second host material according to one embodiment includes a compound represented by the following formula 11.

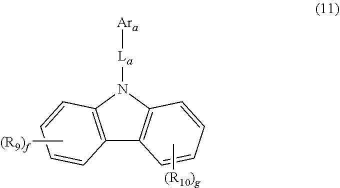

In formula 11,

Lₐ represents a single bond, a substituted or unsubstituted (C6-C30)arylene, or a substituted or unsubstituted (3- to 30-membered)heteroarylene;

Arₐ represents a substituted or unsubstituted (C6-C30)aryl, or a substituted or unsubstituted (3- to 30-membered)heteroaryl;

R₉ and R₁₀ each independently represent hydrogen, deuterium, halogen, cyano, a substituted or unsubstituted (C1-C30)alkyl, a substituted or unsubstituted (C6-C30)aryl, a substituted or unsubstituted (3- to 50-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; or adjacent substituent(s) may be linked to each other to form a ring(s); and

f and g each independently represent an integer of 1 to 4; and when f and g are 2 or more, each of R₉ and each of R₁₀ may be the same or different.

The second host material represented by formula 11 according to one embodiment may be represented by the following formula 12 or 13.

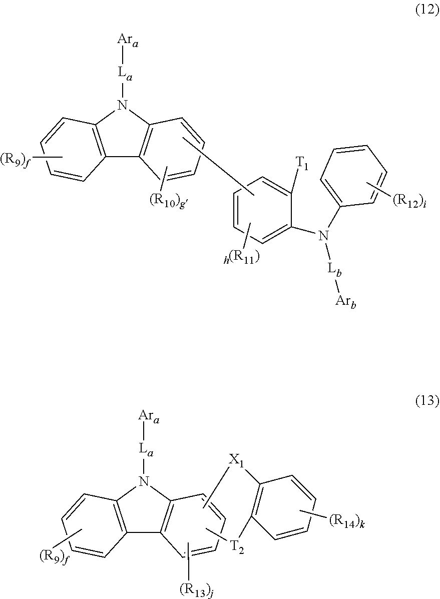

In formulas 12 and 13,

Lₐ, Arₐ, R₉, R₁₀, and f are as defined in formula 11 above;

T₁ and T₂ each independently represent a single bond. O, or S;

L_{b} is as defined as L₈ in formula 11 above;

Ar_{b} is as defined as Arₐ in formula 11 above;

R₁₁ to R₁₄ each independently are defined as R₉ in formula 11 above;

X₁ represents O, S, or NRₐ;

Rₐ represents a substituted or unsubstituted (C6-C30)aryl; and

g′ and h each independently represent an integer of 1 to 3, i and k each independently represent an integer of 1 to 4, and j represents an integer of 1 or 2; and when g′, h, i, j, and k are 2 or more, each of R₁₀, each of R₁₁, each of R₁₂, each of R₁₃, and each of R₁₄ may be the same or different.

In one embodiment, Lₐ and L_{b} each independently may be a single bond or a substituted or unsubstituted (C6-C30)arylene, preferably, a single bond or a substituted or unsubstituted (C6-C25)arylene, more preferably, a single bond or a substituted or unsubstituted (C6-C18)arylene. For example, Lₐ and L_{b} each independently may be a single bond, phenylene, or biphenylene.

In one embodiment, Arₐ and Ar_{b} each independently may be a substituted or unsubstituted (C6-C30)aryl, preferably, a substituted or unsubstituted (C6-C25)aryl, more preferably. (C6-C25)aryl unsubstituted or substituted with (C6-C30)aryl or (5- to 30-membered)heteroaryl. For example, Arₐ and Ar_{b} each independently may be phenyl unsubstituted or substituted with at least one of methyl; cyano; triphenylsilane; phenyl; biphenyl; naphthyl; and carbazolyl unsubstituted or substituted with phenyl, a substituted or unsubstituted o-biphenyl, a substituted or unsubstituted m-biphenyl, a substituted or unsubstituted p-terphenyl, a substituted or unsubstituted m-terphenyl, a substituted or unsubstituted o-terphenyl, a substituted or unsubstituted fluorenyl, naphthyl unsubstituted or substituted with phenyl, or a substituted or unsubstituted triphenylenyl.

In one embodiment, Rₐ may be a substituted or unsubstituted (C6-C30)aryl, preferably, a substituted or unsubstituted (C6-C25)aryl, more preferably, (C6-C25)aryl unsubstituted or substituted with (C6-C30)aryl or (5- to 30-membered)heteroaryl. For example, Rₐ may be phenyl unsubstituted or substituted with at least one of phenyl; biphenyl; naphthyl; and carbazolyl unsubstituted or substituted with phenyl, a substituted or unsubstituted o-biphenyl, a substituted or unsubstituted m-biphenyl, a substituted or unsubstituted p-terphenyl, a substituted or unsubstituted m-terphenyl, a substituted or unsubstituted o-terphenyl, naphthyl unsubstituted or substituted with phenyl, or a substituted or unsubstituted triphenylenyl.

In one embodiment, R₉ to R₁₄ each independently may be hydrogen, a substituted or unsubstituted (C1-C30)alkyl, a substituted or unsubstituted (C6-C30)aryl, or a substituted or unsubstituted (3- to 30-membered)heteroaryl, preferably, hydrogen, a substituted or unsubstituted (C1-C10)alkyl, a substituted or unsubstituted (C6-C25)aryl, or a substituted or unsubstituted (5- to 25-membered)heteroaryl, more preferably, hydrogen, a substituted or unsubstituted (C1-C4)alkyl, a substituted or unsubstituted (C6-C18)aryl, or a substituted or unsubstituted (5- to 18-membered)heteroaryl. For example, R₉ to R₁₄ each independently may be hydrogen, a substituted or unsubstituted methyl, a substituted or unsubstituted phenyl, or a substituted or unsubstituted carbazolyl.

According to one embodiment, the compound represented by formula 11 may be more specifically exemplified by the following compounds, but is not limited thereto.

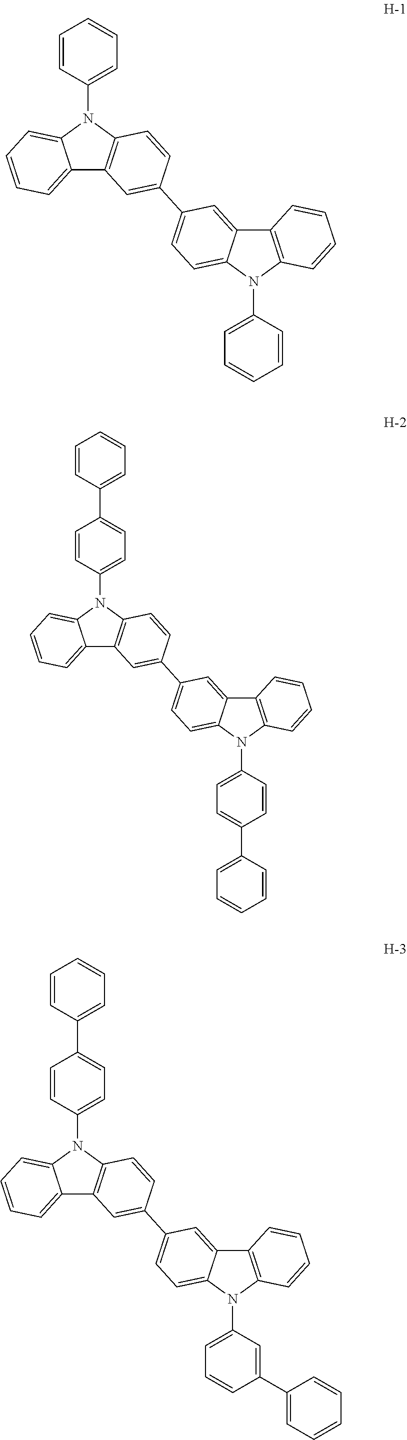 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

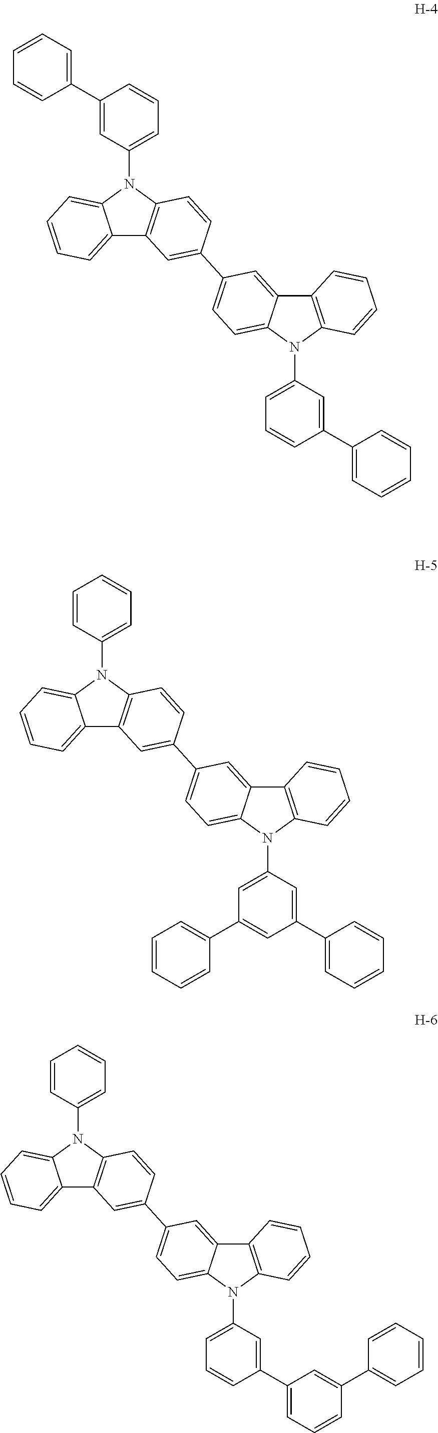 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1]

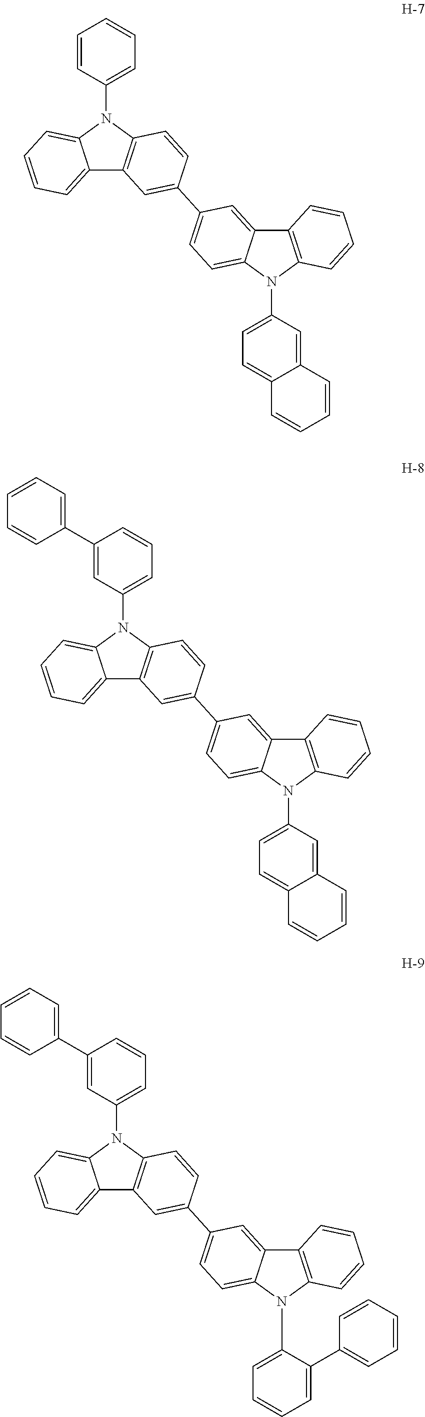 [SMILES: c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1]

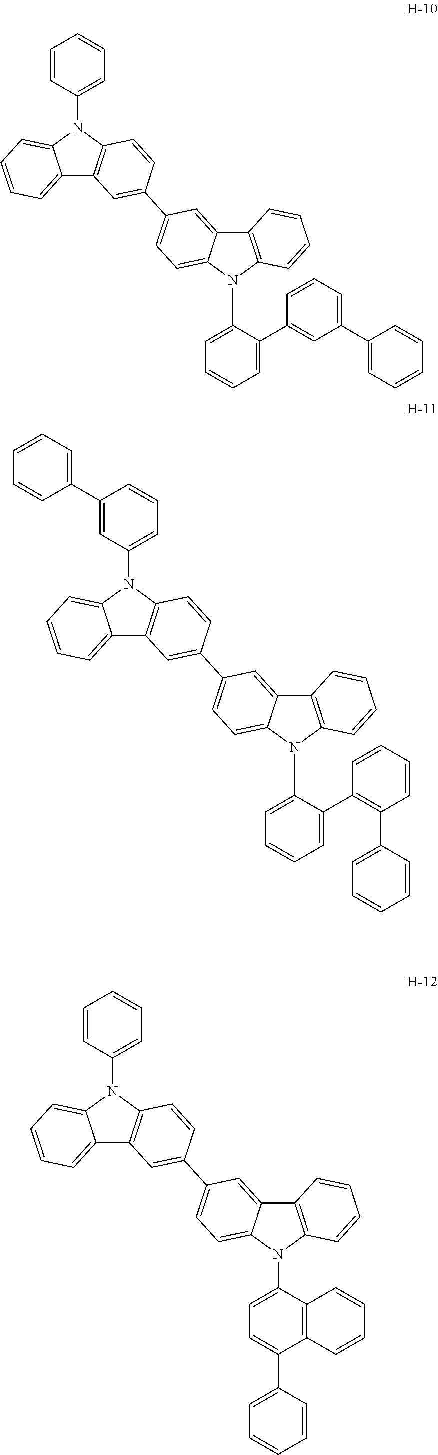 [SMILES: c1ccc(-c2cccc(-c3ccccc3-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccccc5-c5ccccc5-c5ccccc5)ccc43)c2)cc1 ; c1ccc(-c2ccc(-n3c4ccccc4c4cc(-c5ccc6c(c5)c5ccccc5n6-c5ccccc5)ccc43)c3ccccc23)cc1]

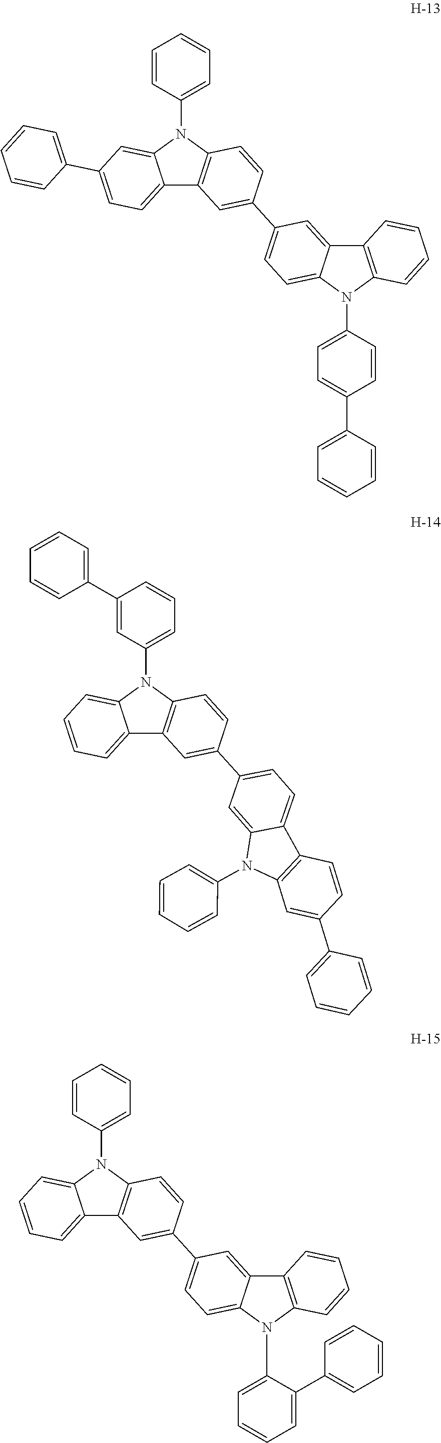 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2cccc(-n3c4ccccc4c4cc(-c5ccc6c7ccc(-c8ccccc8)cc7n(-c7ccccc7)c6c5)ccc43)c2)cc1 ; c1ccc(-c2ccccc2-n2c3ccccc3c3cc(-c4ccc5c(c4)c4ccccc4n5-c4ccccc4)ccc32)cc1]

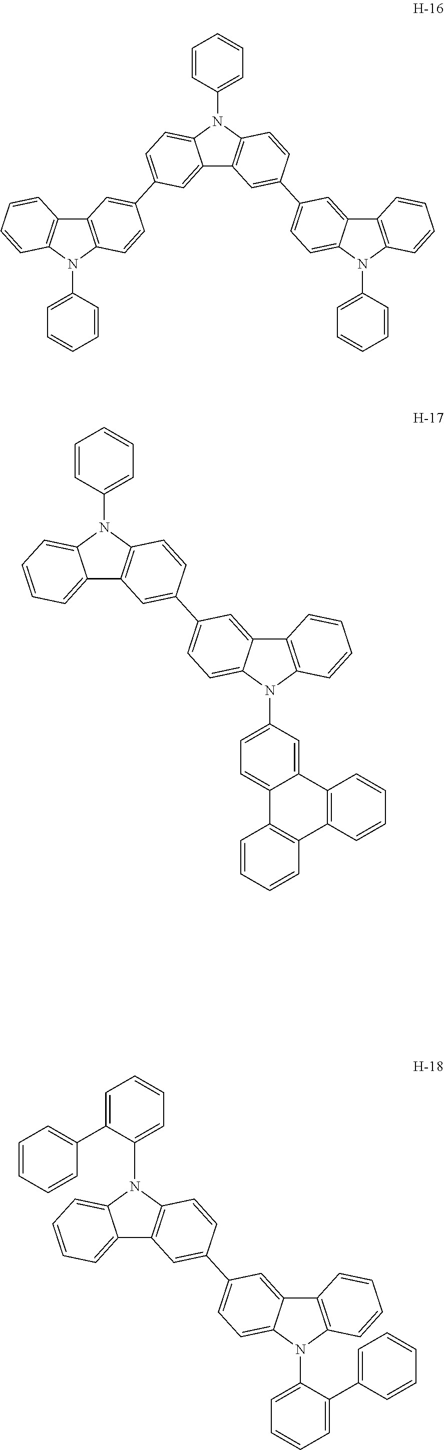 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; c1ccc(-c2ccccc2-n2c3ccccc3c3cc(-c4ccc5c(c4)c4ccccc4n5-c4ccccc4-c4ccccc4)ccc32)cc1]

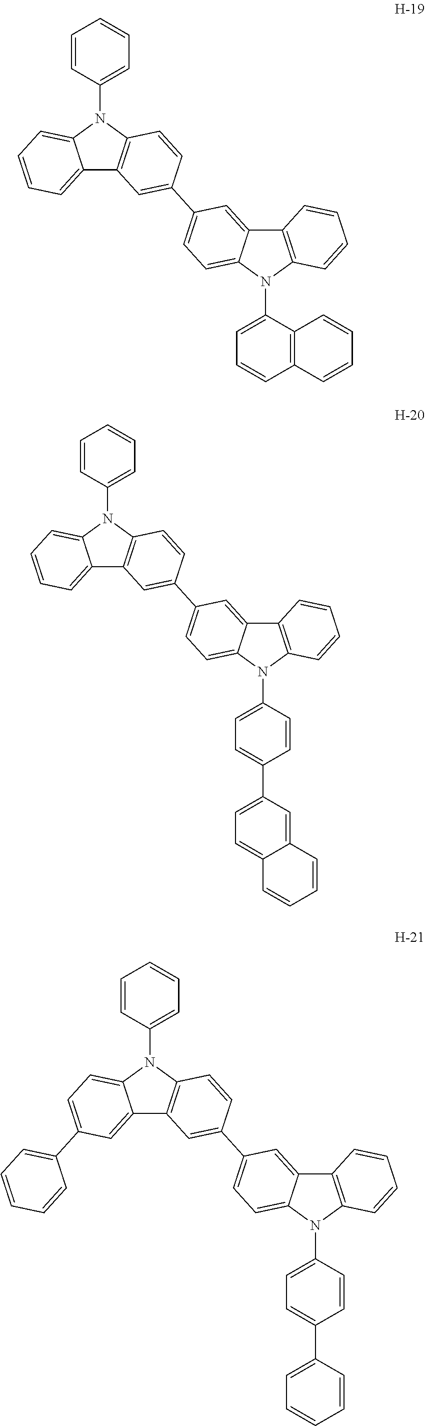 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

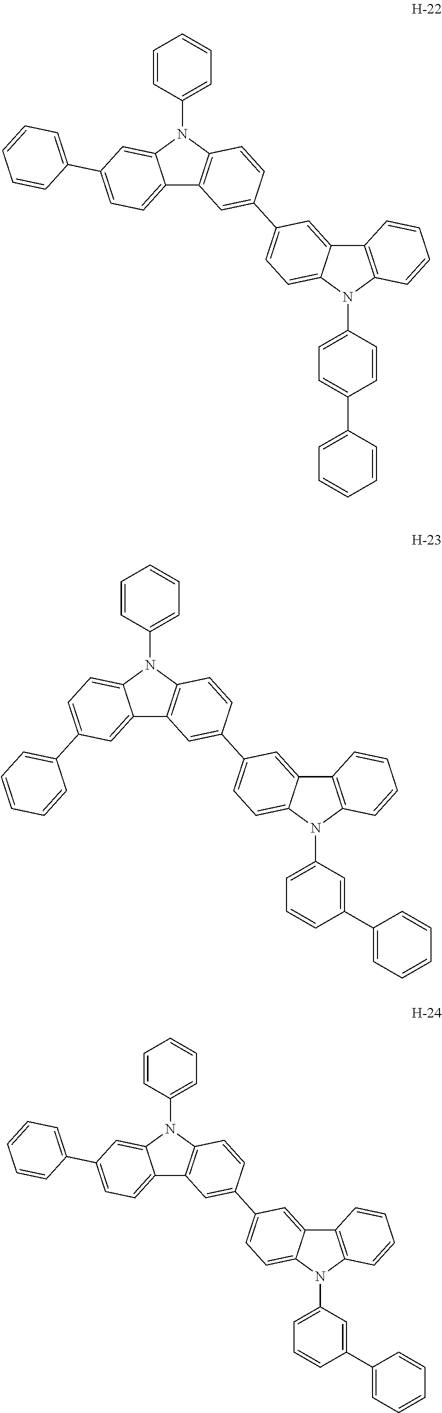 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-c2cccc(-n3c4ccccc4c4cc(-c5ccc6c(c5)c5ccc(-c7ccccc7)cc5n6-c5ccccc5)ccc43)c2)cc1]

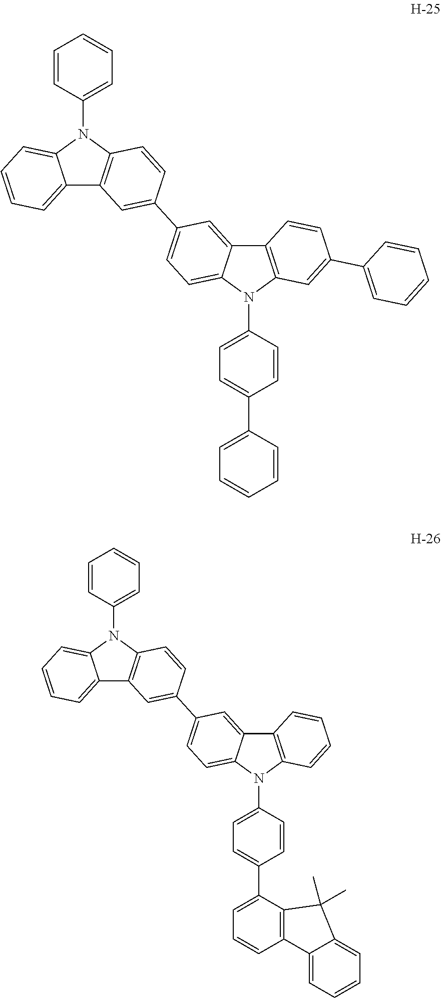 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; CC1(C)c2ccccc2-c2cccc(-c3ccc(-n4c5ccccc5c5cc(-c6ccc7c(c6)c6ccccc6n7-c6ccccc6)ccc54)cc3)c21]

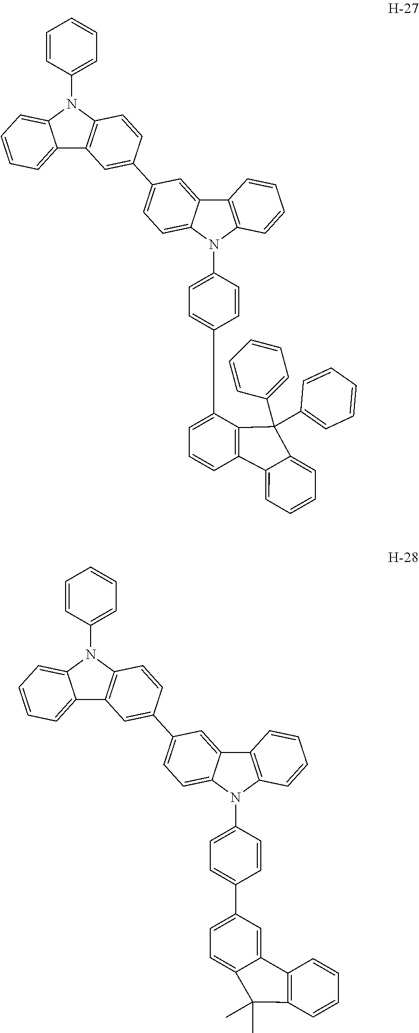 [SMILES: c1ccc(-n2c3ccccc3c3cc(-c4ccc5c(c4)c4ccccc4n5-c4ccc(-c5cccc6c5C(c5ccccc5)(c5ccccc5)c5ccccc5-6)cc4)ccc32)cc1 ; CC1(C)c2ccccc2-c2cc(-c3ccc(-n4c5ccccc5c5cc(-c6ccc7c(c6)c6ccccc6n7-c6ccccc6)ccc54)cc3)ccc21]

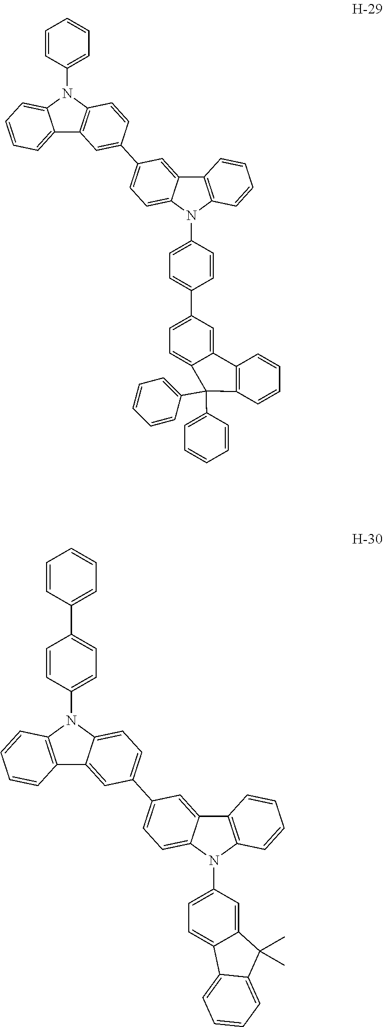 [SMILES: c1ccc(-n2c3ccccc3c3cc(-c4ccc5c(c4)c4ccccc4n5-c4ccc(-c5ccc6c(c5)-c5ccccc5C6(c5ccccc5)c5ccccc5)cc4)ccc32)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

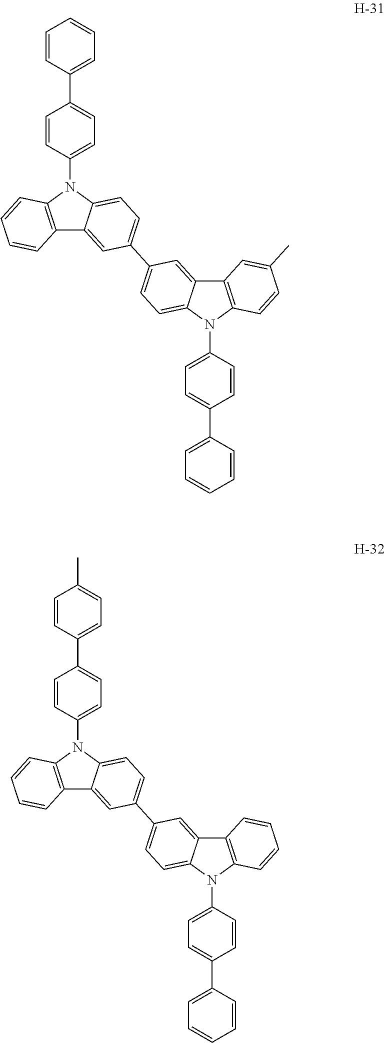 [SMILES: Cc1ccc2c(c1)c1cc(-c3ccc4c(c3)c3ccccc3n4-c3ccc(-c4ccccc4)cc3)ccc1n2-c1ccc(-c2ccccc2)cc1 ; Cc1ccc(-c2ccc(-n3c4ccccc4c4cc(-c5ccc6c(c5)c5ccccc5n6-c5ccc(-c6ccccc6)cc5)ccc43)cc2)cc1]

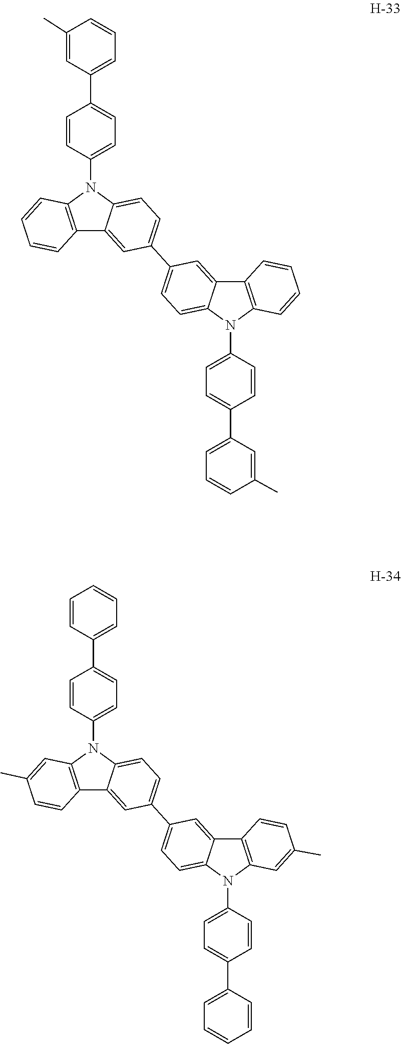

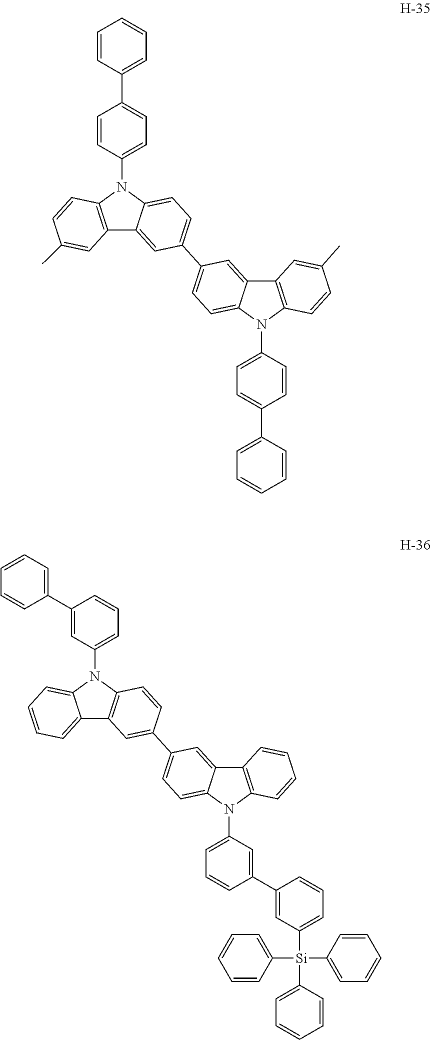

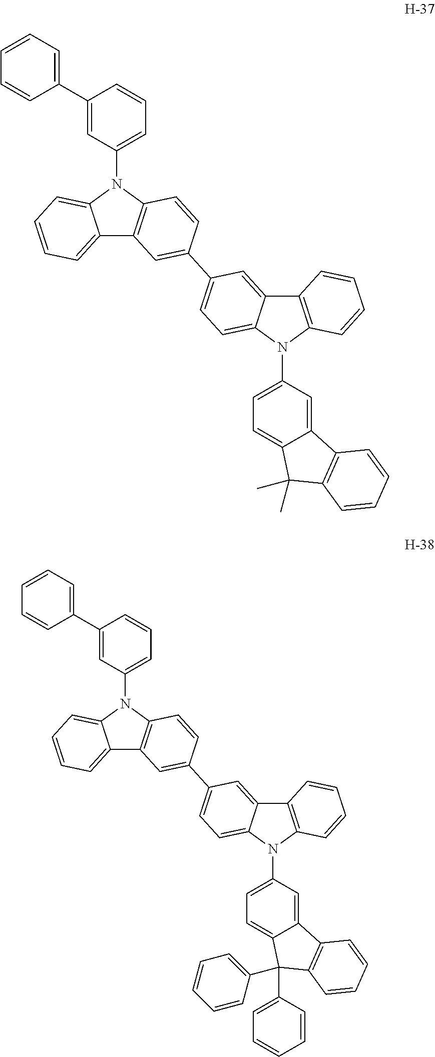 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; c1ccc(-c2cccc(-n3c4ccccc4c4cc(-c5ccc6c(c5)c5ccccc5n6-c5ccc6c(c5)-c5ccccc5C6(c5ccccc5)c5ccccc5)ccc43)c2)cc1]

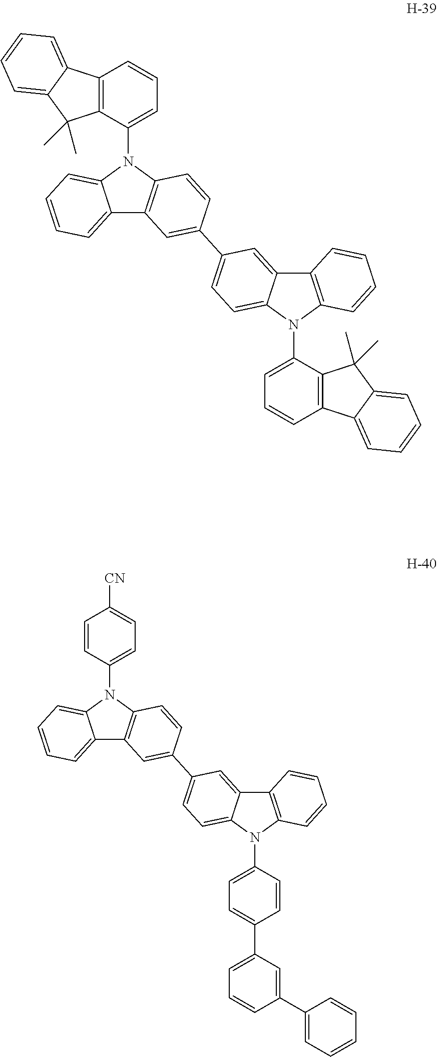 [SMILES: CC1(C)c2ccccc2-c2cccc(-n3c4ccccc4c4cc(-c5ccc6c(c5)c5ccccc5n6-c5cccc6c5C(C)(C)c5ccccc5-6)ccc43)c21 ; N#Cc1ccc(-n2c3ccccc3c3cc(-c4ccc5c(c4)c4ccccc4n5-c4ccc(-c5cccc(-c6ccccc6)c5)cc4)ccc32)cc1]

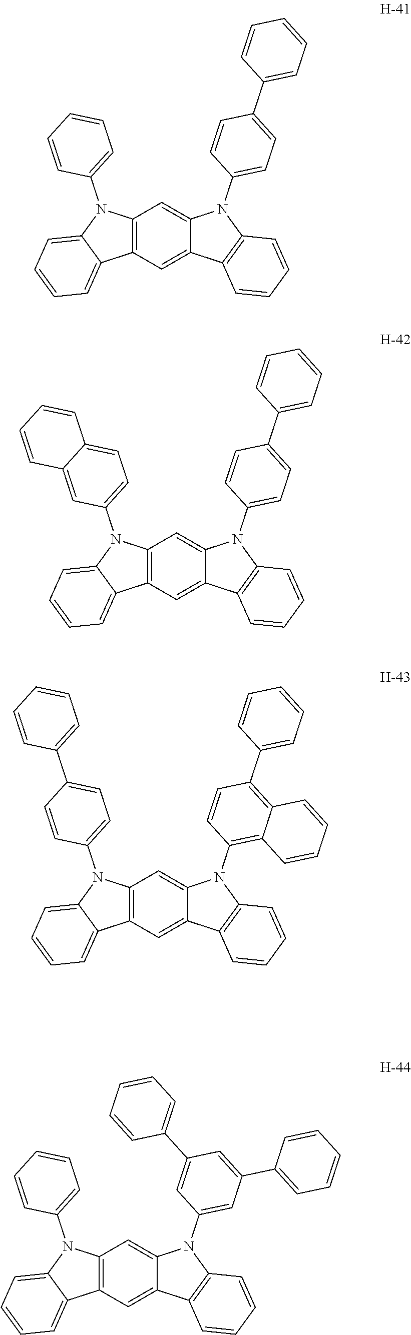 [SMILES: c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc7ccccc7c6)c5cc43)cc2)cc1 ; c1ccc(-c2ccc(-n3c4ccccc4c4cc5c6ccccc6n(-c6ccc(-c7ccccc7)c7ccccc67)c5cc43)cc2)cc1 ; c1ccc(-c2cc(-c3ccccc3)cc(-n3c4ccccc4c4cc5c6ccccc6n(-c6ccccc6)c5cc43)c2)cc1]

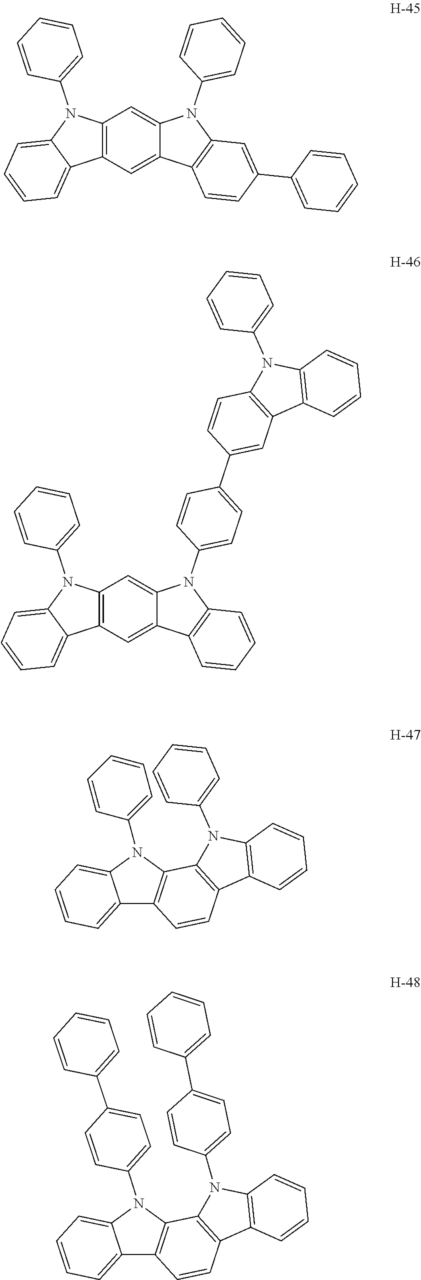 [SMILES: c1ccc(-c2ccc3c4cc5c6ccccc6n(-c6ccccc6)c5cc4n(-c4ccccc4)c3c2)cc1 ; c1ccc(-n2c3ccccc3c3cc(-c4ccc(-n5c6ccccc6c6cc7c8ccccc8n(-c8ccccc8)c7cc65)cc4)ccc32)cc1 ; c1ccc(-n2c3ccccc3c3ccc4c5ccccc5n(-c5ccccc5)c4c32)cc1 ; c1ccc(-c2ccc(-n3c4ccccc4c4ccc5c6ccccc6n(-c6ccc(-c7ccccc7)cc6)c5c43)cc2)cc1]

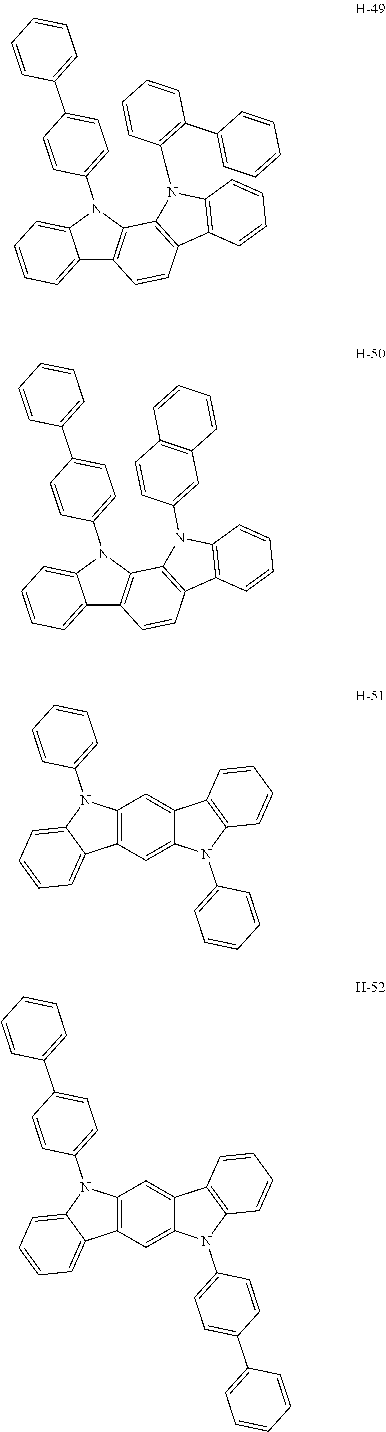 [SMILES: c1ccc(-c2ccc(-n3c4ccccc4c4ccc5c6ccccc6n(-c6ccccc6-c6ccccc6)c5c43)cc2)cc1 ; c1ccc(-c2ccc(-n3c4ccccc4c4ccc5c6ccccc6n(-c6ccc7ccccc7c6)c5c43)cc2)cc1 ; c1ccc(-n2c3ccccc3c3cc4c(cc32)c2ccccc2n4-c2ccccc2)cc1 ; c1ccc(-c2ccc(-n3c4ccccc4c4cc5c(cc43)c3ccccc3n5-c3ccc(-c4ccccc4)cc3)cc2)cc1]

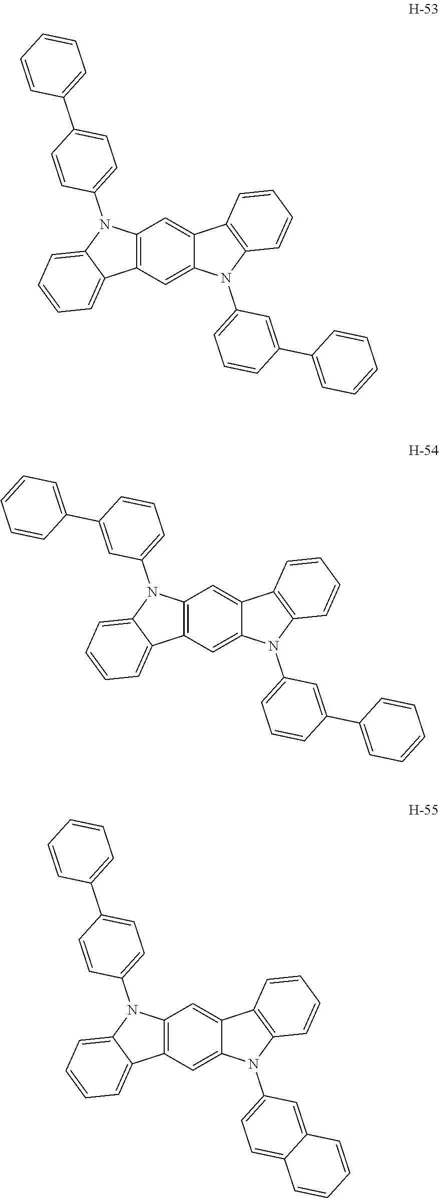 [SMILES: c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1 ; c1ccc(-c2ccc(-n3c4ccccc4c4cc5c(cc43)c3ccccc3n5-c3ccc4ccccc4c3)cc2)cc1]

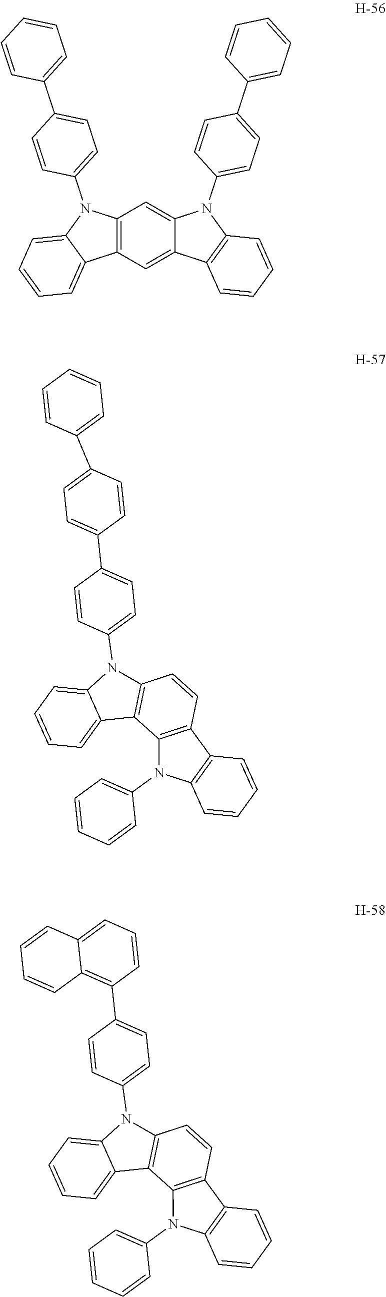 [SMILES: c1ccc(-c2ccc(-n3c4ccccc4c4cc5c6ccccc6n(-c6ccc(-c7ccccc7)cc6)c5cc43)cc2)cc1 ; c1ccc(-c2ccc(-c3ccc(-n4c5ccccc5c5c4ccc4c6ccccc6n(-c6ccccc6)c45)cc3)cc2)cc1 ; c1ccc(-n2c3ccccc3c3ccc4c(c5ccccc5n4-c4ccc(-c5cccc6ccccc56)cc4)c32)cc1]

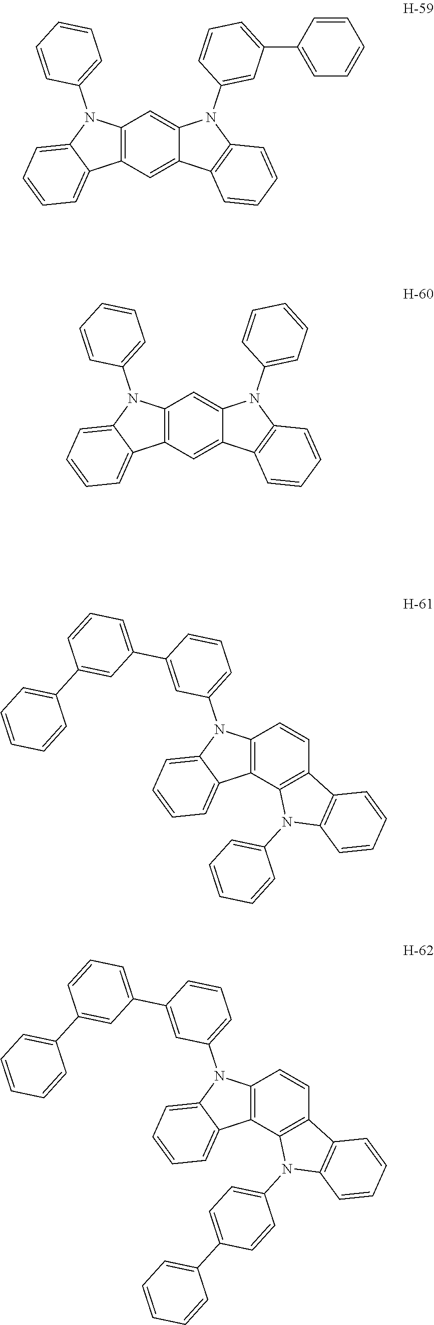 [SMILES: c1ccc(-c2cccc(-n3c4ccccc4c4cc5c6ccccc6n(-c6ccccc6)c5cc43)c2)cc1 ; c1ccc(-n2c3ccccc3c3cc4c5ccccc5n(-c5ccccc5)c4cc32)cc1 ; c1ccc(-c2cccc(-c3cccc(-n4c5ccccc5c5c4ccc4c6ccccc6n(-c6ccccc6)c45)c3)c2)cc1 ; c1ccc(-c2ccc(-n3c4ccccc4c4ccc5c(c6ccccc6n5-c5cccc(-c6cccc(-c7ccccc7)c6)c5)c43)cc2)cc1]

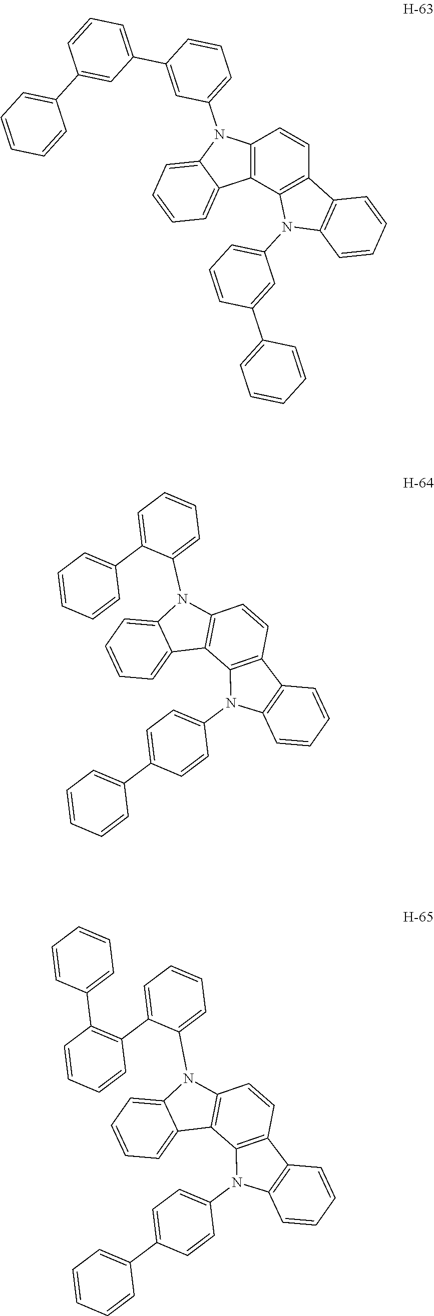 [SMILES: c1ccc(-c2cccc(-c3cccc(-n4c5ccccc5c5c4ccc4c6ccccc6n(-c6cccc(-c7ccccc7)c6)c45)c3)c2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccccc5-c5ccccc5-c5ccccc5)c43)cc2)cc1]

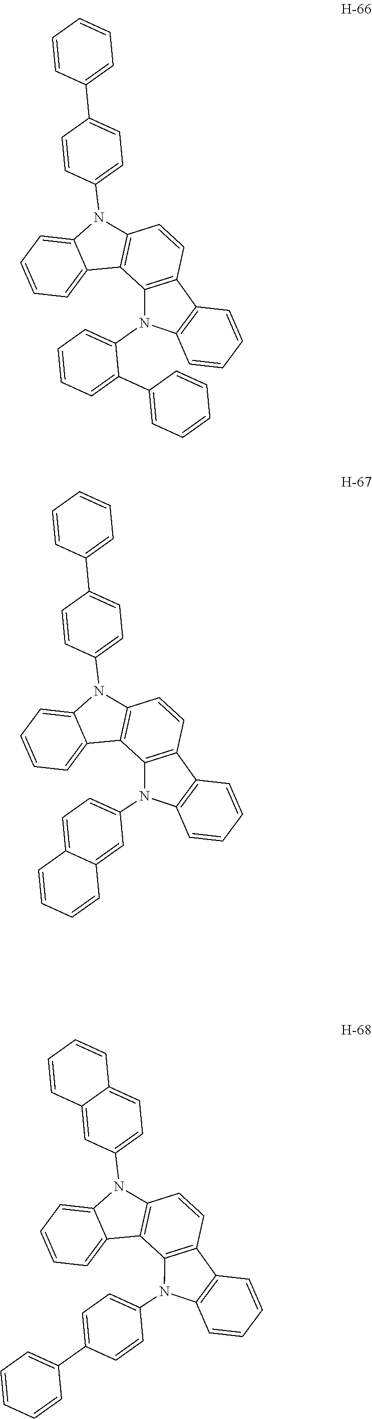 [SMILES: c1ccc(-c2ccc(-n3c4ccccc4c4c3ccc3c5ccccc5n(-c5ccccc5-c5ccccc5)c34)cc2)cc1 ; c1ccc(-c2ccc(-n3c4ccccc4c4c3ccc3c5ccccc5n(-c5ccc6ccccc6c5)c34)cc2)cc1 ; c1ccc(-c2ccc(-n3c4ccccc4c4ccc5c(c6ccccc6n5-c5ccc6ccccc6c5)c43)cc2)cc1]

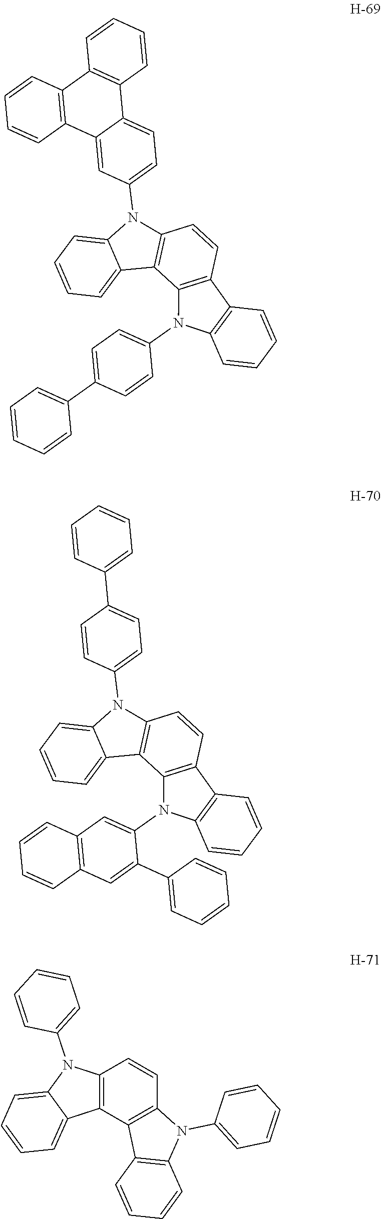 [SMILES: c1ccc(-c2ccc(-n3c4ccccc4c4ccc5c(c6ccccc6n5-c5ccc6c7ccccc7c7ccccc7c6c5)c43)cc2)cc1 ; c1ccc(-c2ccc(-n3c4ccccc4c4c3ccc3c5ccccc5n(-c5cc6ccccc6cc5-c5ccccc5)c34)cc2)cc1 ; c1ccc(-n2c3ccccc3c3c4c5ccccc5n(-c5ccccc5)c4ccc32)cc1]

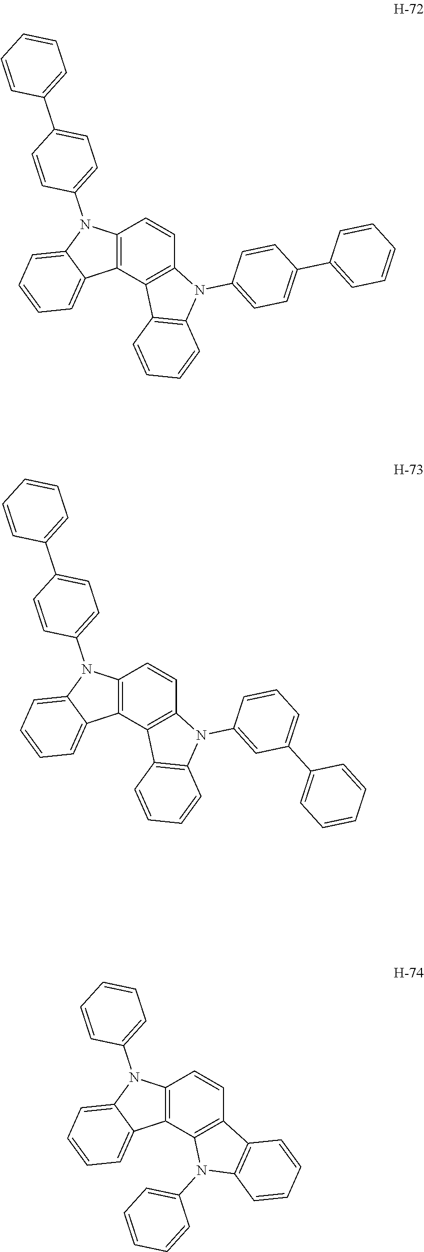 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-n2c3ccccc3c3c2ccc2c4ccccc4n(-c4ccccc4)c23)cc1]

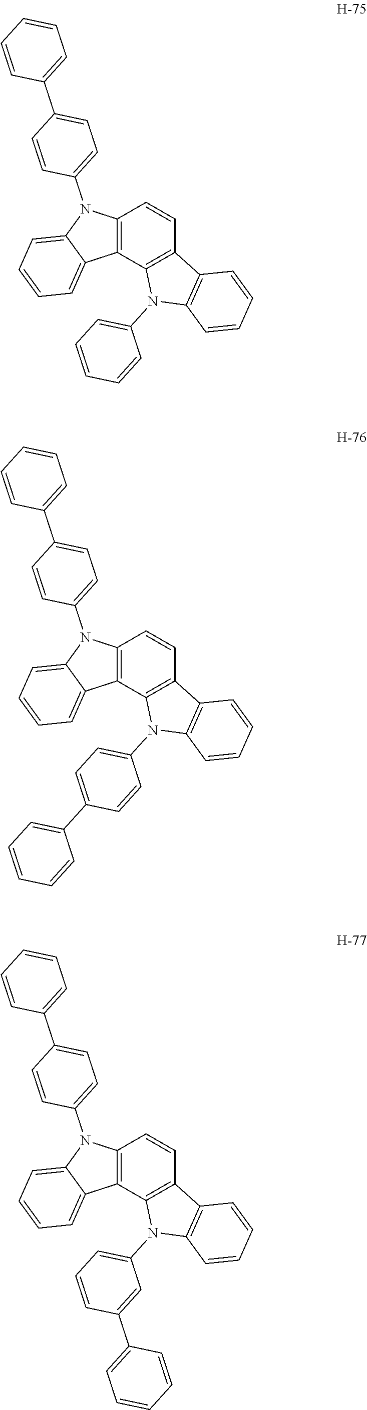 [SMILES: c1ccc(-c2ccc(-n3c4ccccc4c4c3ccc3c5ccccc5n(-c5ccccc5)c34)cc2)cc1 ; c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4c3ccc3c5ccccc5n(-c5cccc(-c6ccccc6)c5)c34)cc2)cc1]

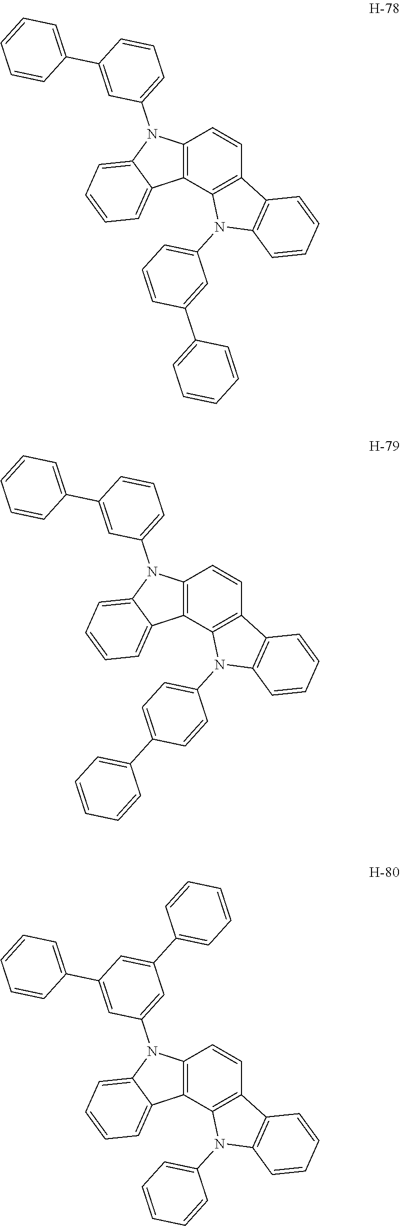 [SMILES: c1ccc(-c2cccc(-n3c4ccccc4c4c3ccc3c5ccccc5n(-c5cccc(-c6ccccc6)c5)c34)c2)cc1 ; c1ccc(-c2ccc(-n3c4ccccc4c4ccc5c(c6ccccc6n5-c5cccc(-c6ccccc6)c5)c43)cc2)cc1 ; c1ccc(-c2cc(-c3ccccc3)cc(-n3c4ccccc4c4c3ccc3c5ccccc5n(-c5ccccc5)c34)c2)cc1]

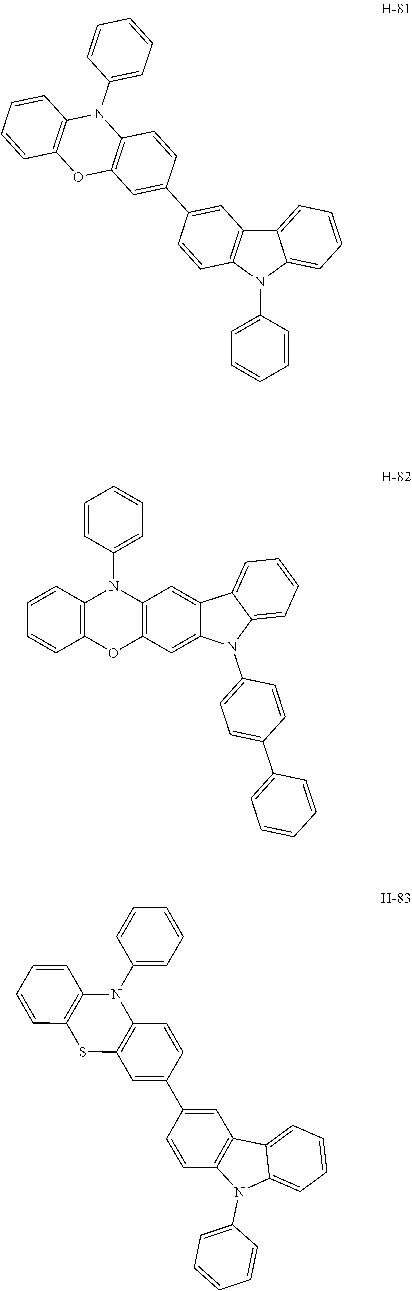 [SMILES: c1ccc(N2c3ccccc3Oc3cc(-c4ccc5c(c4)c4ccccc4n5-c4ccccc4)ccc32)cc1 ; c1ccc(-c2ccc(-n3c4ccccc4c4cc5c(cc43)Oc3ccccc3N5c3ccccc3)cc2)cc1 ; c1ccc(N2c3ccccc3Sc3cc(-c4ccc5c(c4)c4ccccc4n5-c4ccccc4)ccc32)cc1]

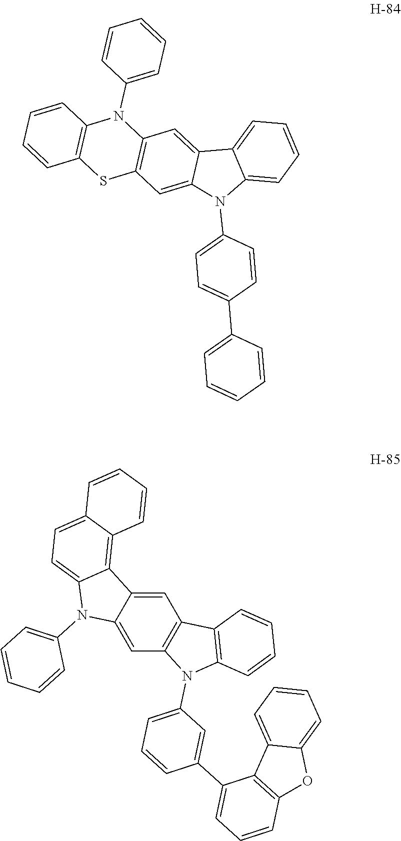 [SMILES: c1ccc(-c2ccc(-n3c4ccccc4c4cc5c(cc43)Sc3ccccc3N5c3ccccc3)cc2)cc1 ; c1ccc(-n2c3cc4c(cc3c3c5ccccc5ccc32)c2ccccc2n4-c2cccc(-c3cccc4oc5ccccc5c34)c2)cc1]

The compound of formula 11 according to the present disclosure may be prepared by a synthetic method known to one skilled in the art.

The dopant comprised in the organic electroluminescent material of the present disclosure may be at least one phosphorescent or fluorescent dopant, preferably, a phosphorescent dopant. The phosphorescent dopant material applied to the present disclosure is not particularly limited, but may be preferably a metallated complex compound(s) of a metal atom(s) selected from iridium (Ir), osmium (Os), copper (Cu), and platinum (Pt), as necessary; more preferably an ortho-metallated complex compound(s) of a metal atom(s) selected from iridium (Ir), osmium (Os), copper (Cu), and platinum (Pt), as necessary; and even more preferably ortho-metallated iridium complex compound(s), as necessary.

The dopant comprised in the organic electroluminescent device of the present disclosure may use the compound represented by the following formula 101, but is not limited thereto:

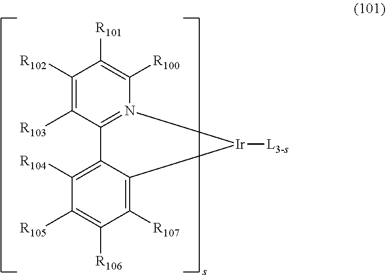

In formula 101,

L is selected from the following structures 1 to 3;

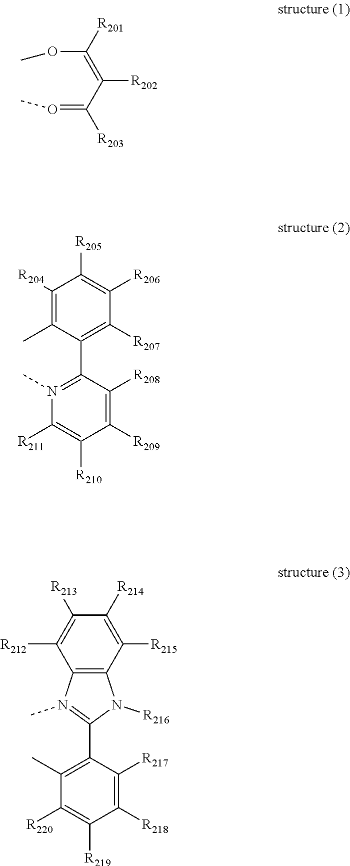

in structures 1 to 3,

R₁₀₀ to R₁₀₃ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or adjacent substituent(s) may be linked to each other to form a ring(s), for example, to form a ring(s) with a pyridine, e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent hydrogen, deuterium, halogen. (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or adjacent substituent(s) may be linked to each other to form a ring(s), for example, to form a ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothieno pyridine;

R₂₀₁ to R₂₂₀ each independently represent hydrogen, deuterium, halogen. (C1-C30)alkyl unsubstituted or substituted with deuterium and/or halogen, a substituted or unsubstituted (C3-C30)cycloalkyl, or a substituted or unsubstituted (C6-C30)aryl; or adjacent substituent(s) may be linked to each other to form a ring(s); and

s represents an integer of 1 to 3.

Specifically, the specific examples of the dopant compound include the following, but are not limited thereto.

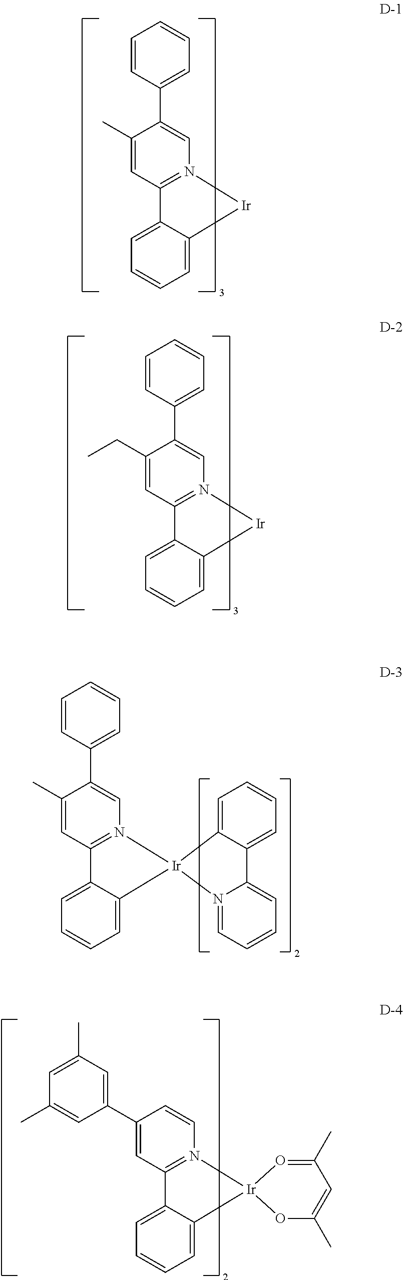

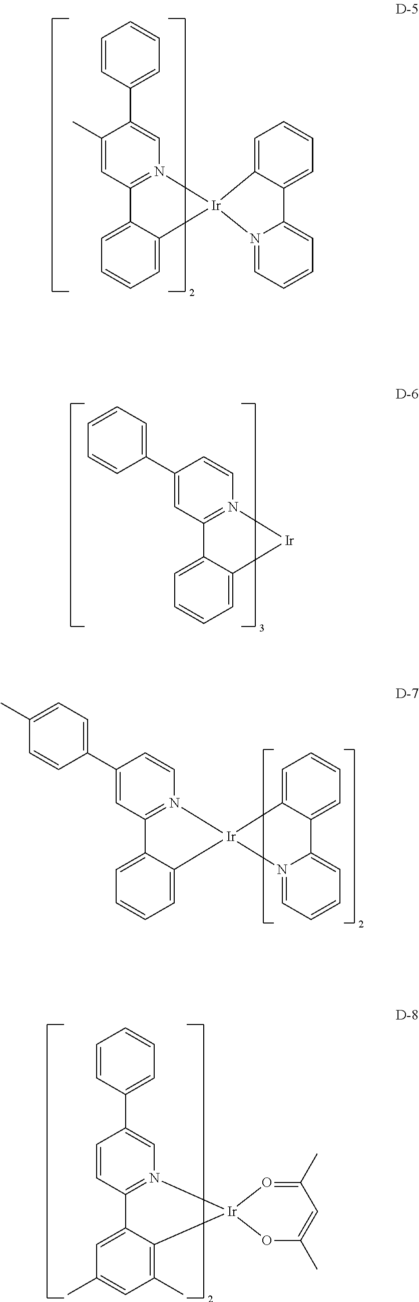

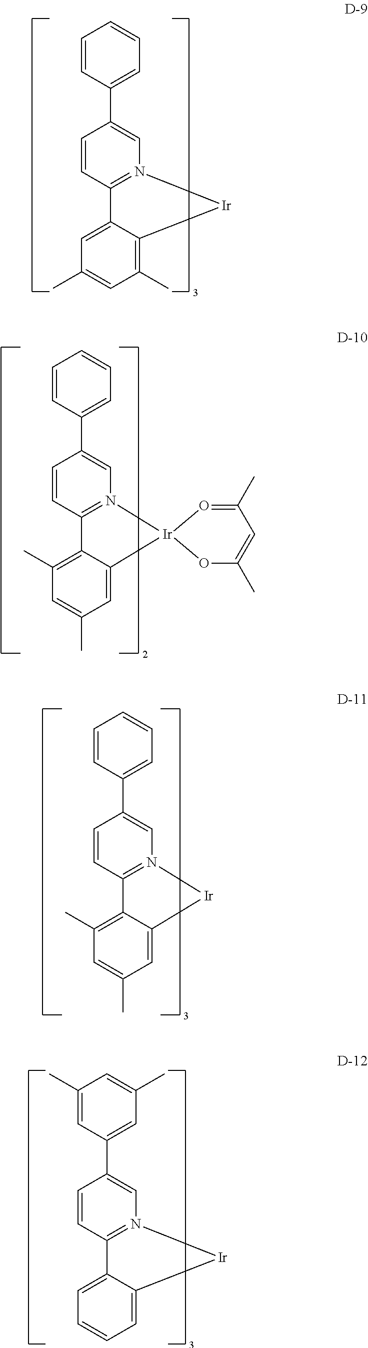

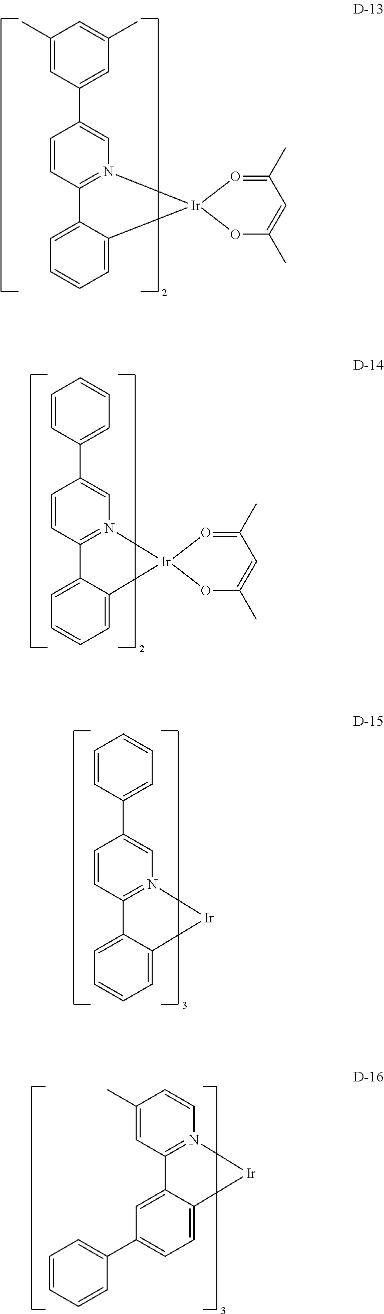

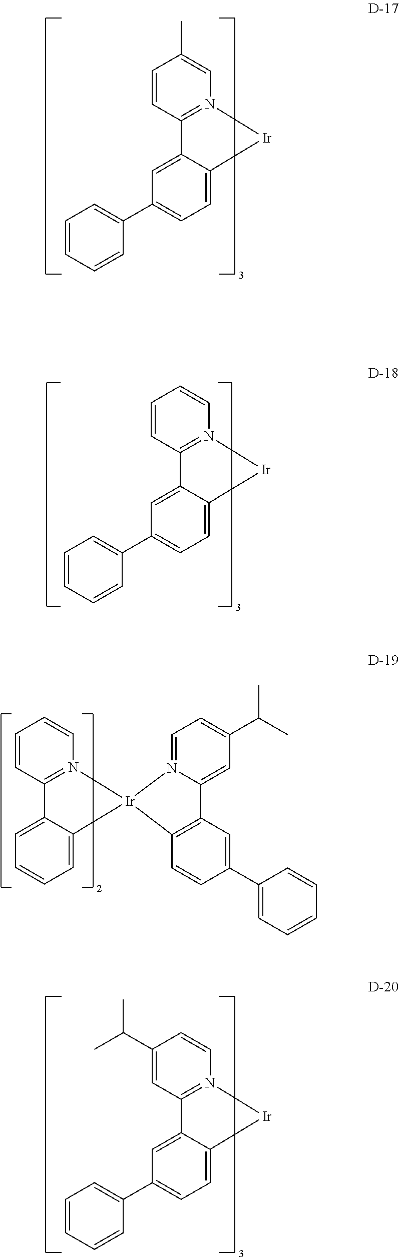

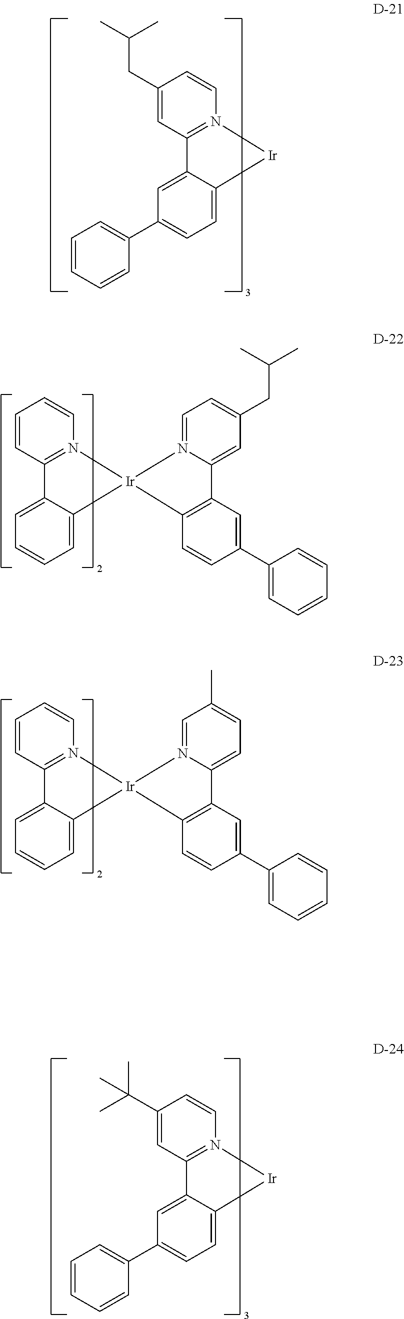

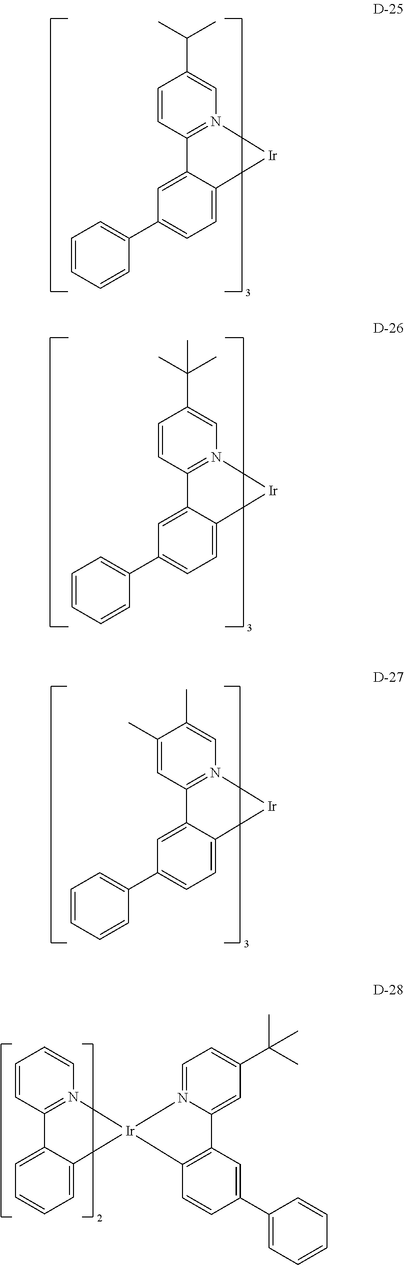

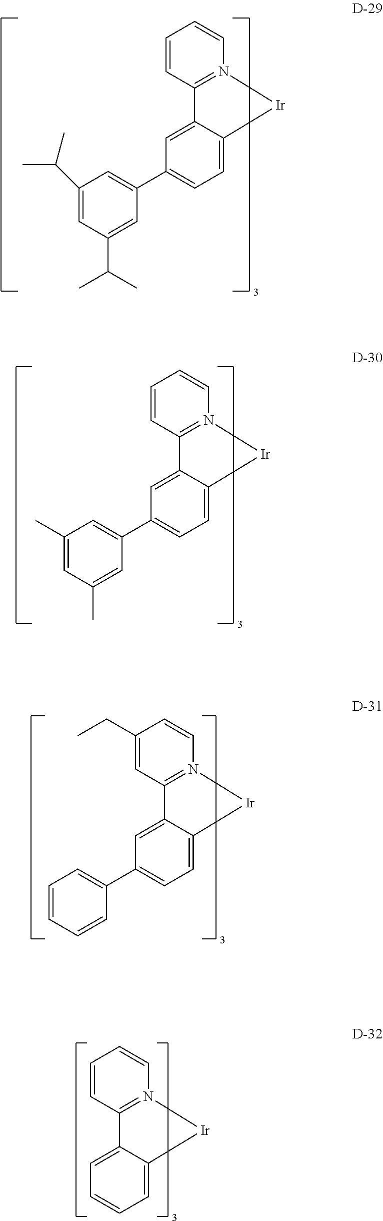

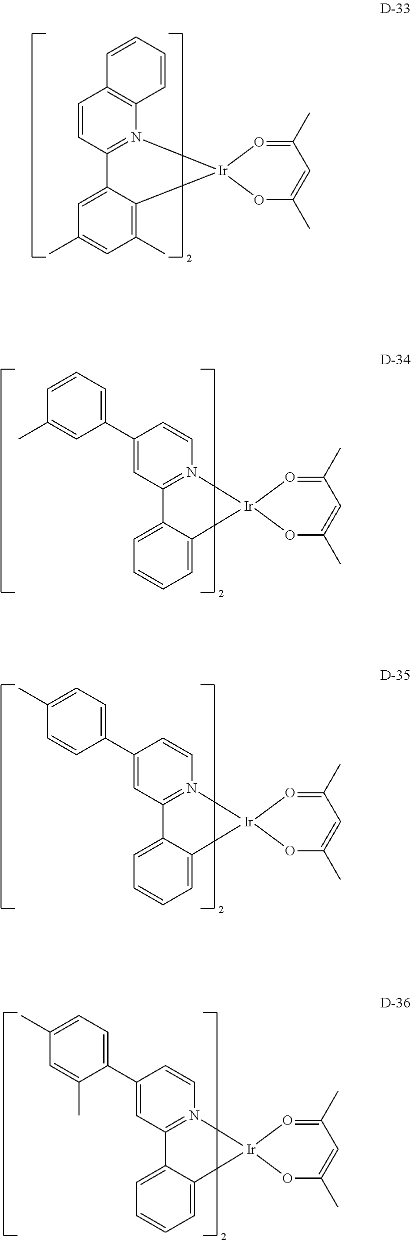

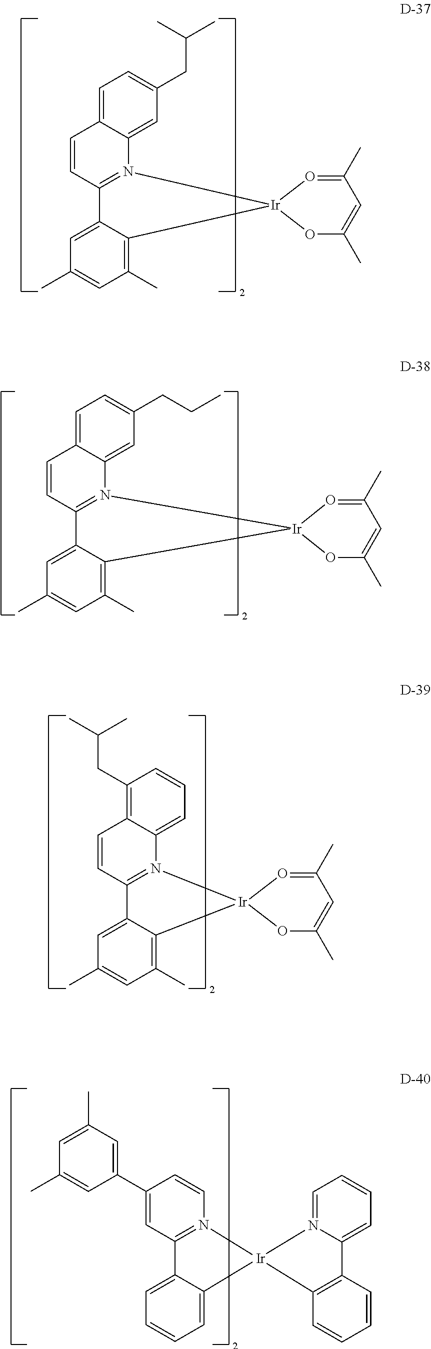

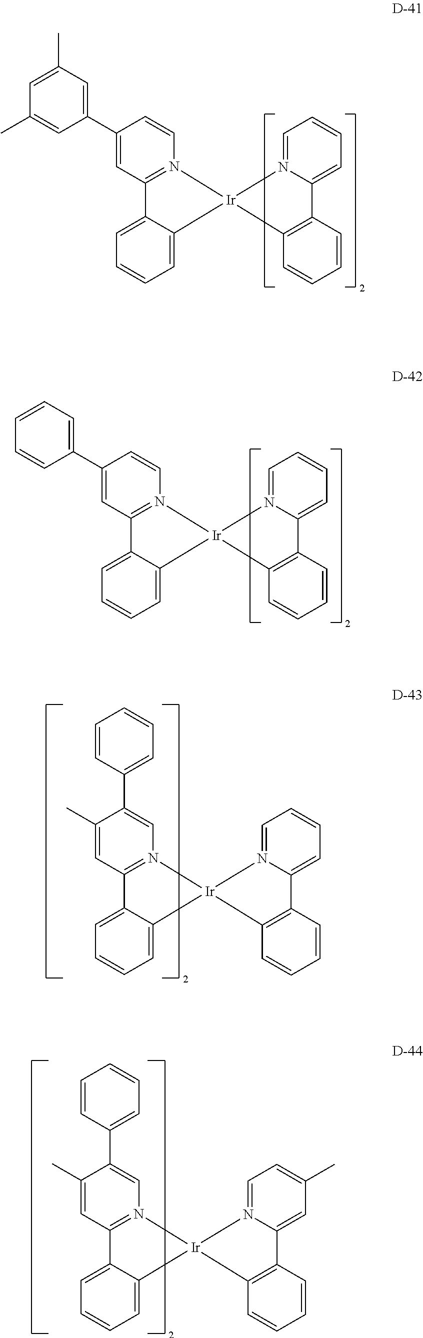

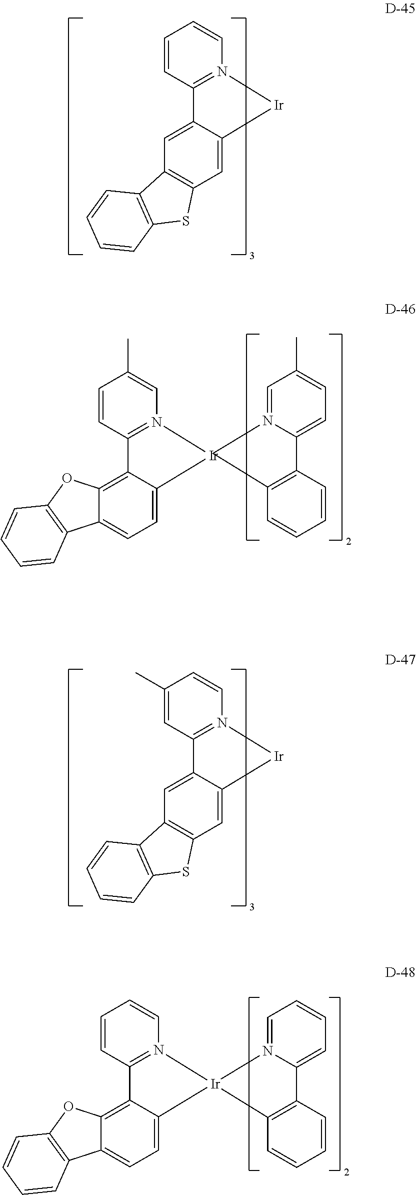

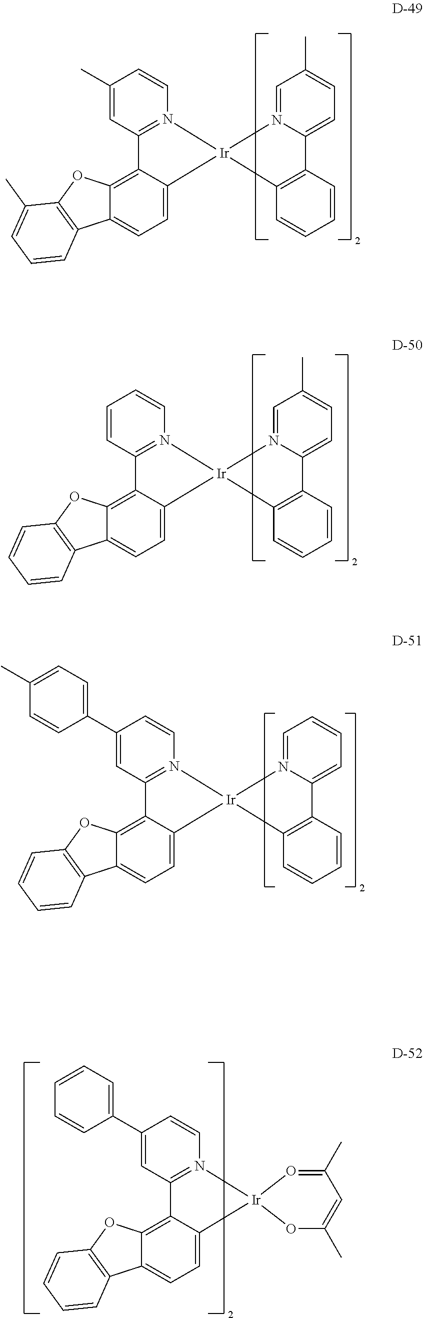

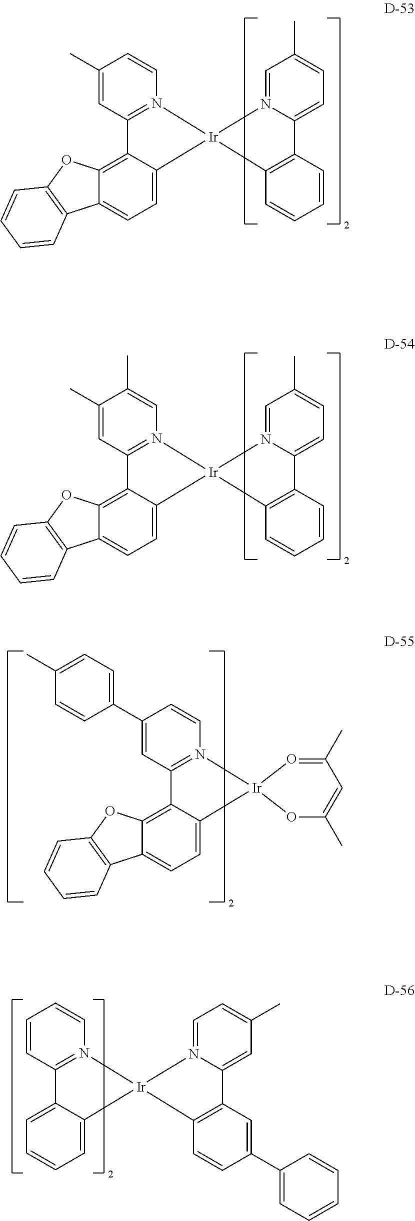

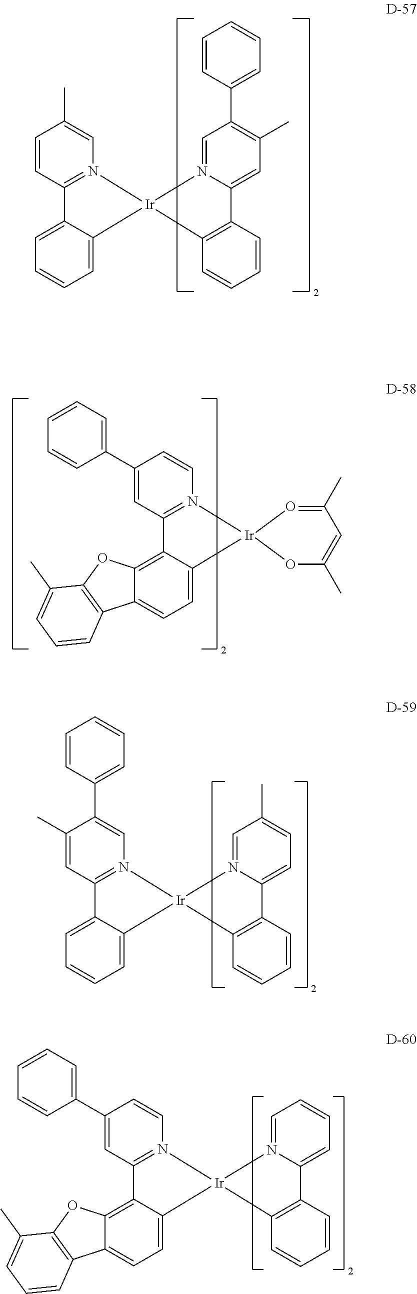

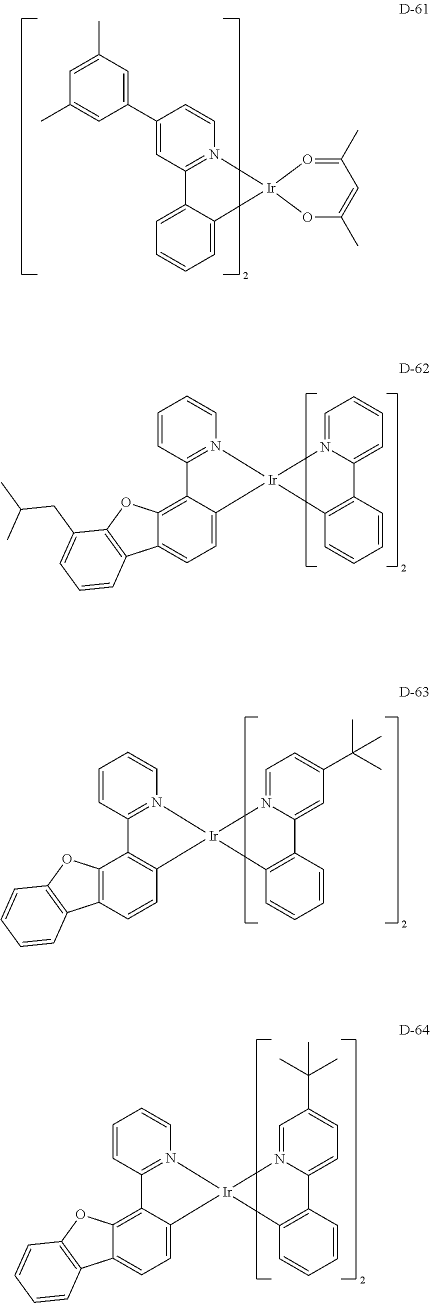

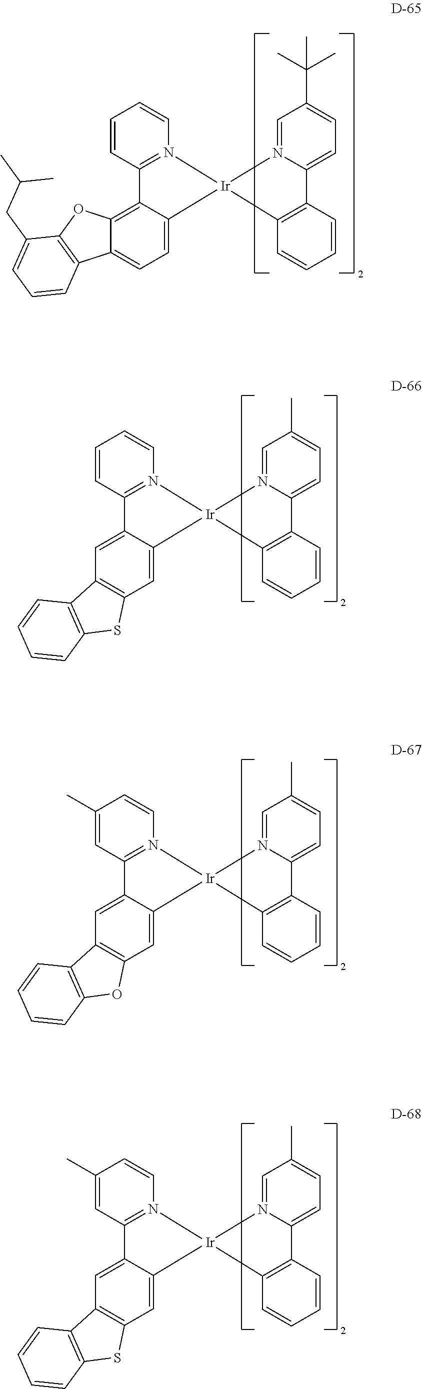

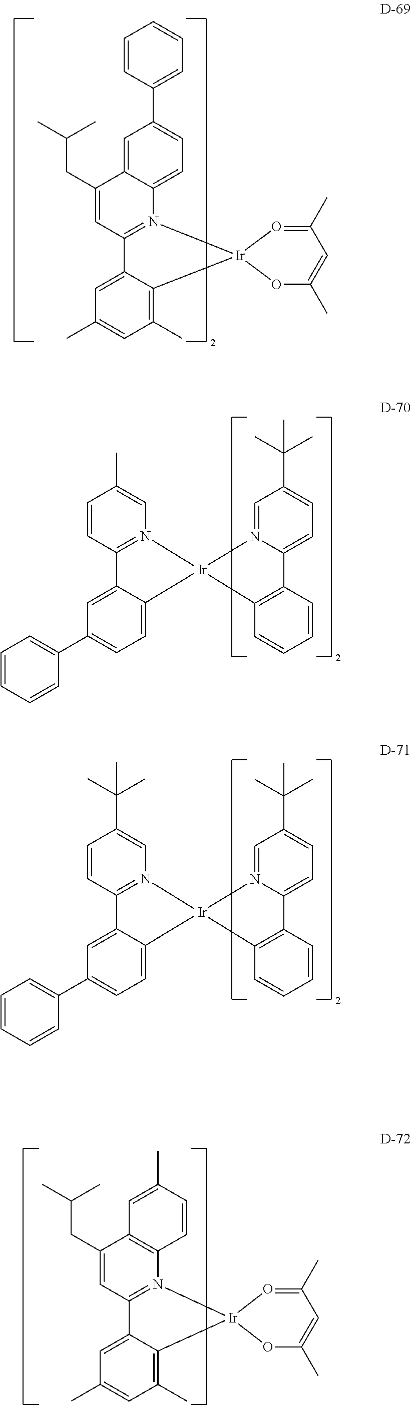

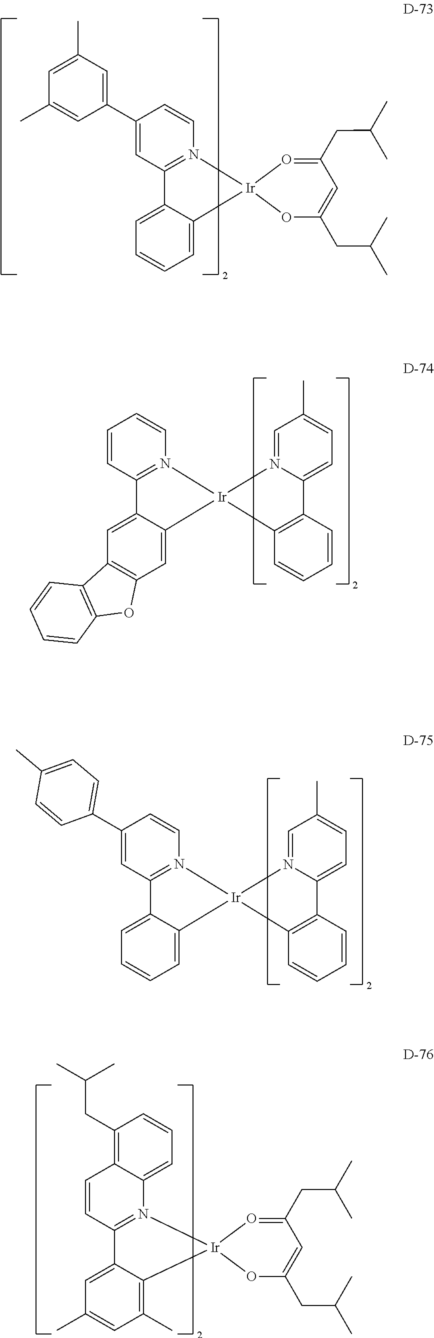

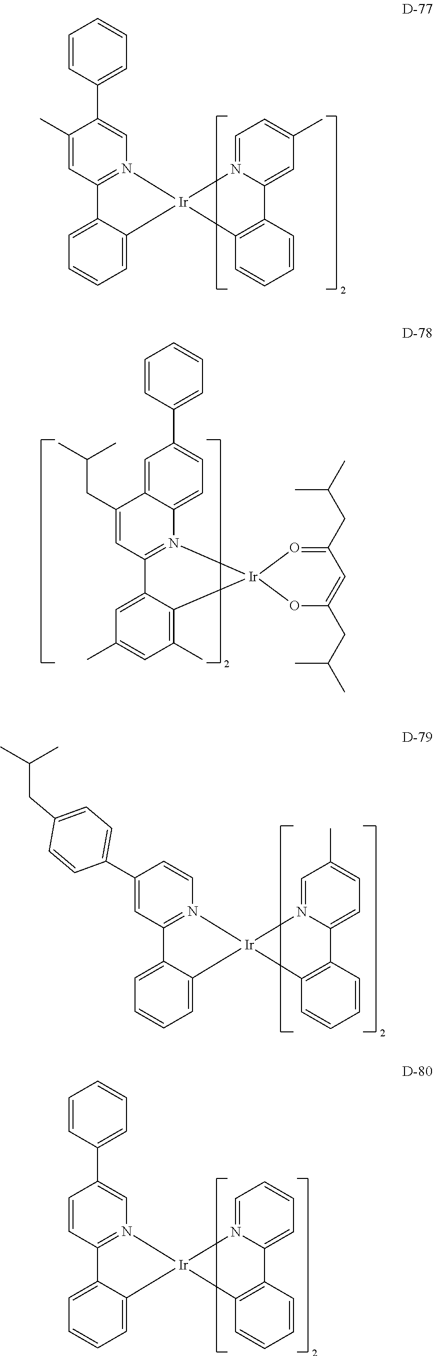

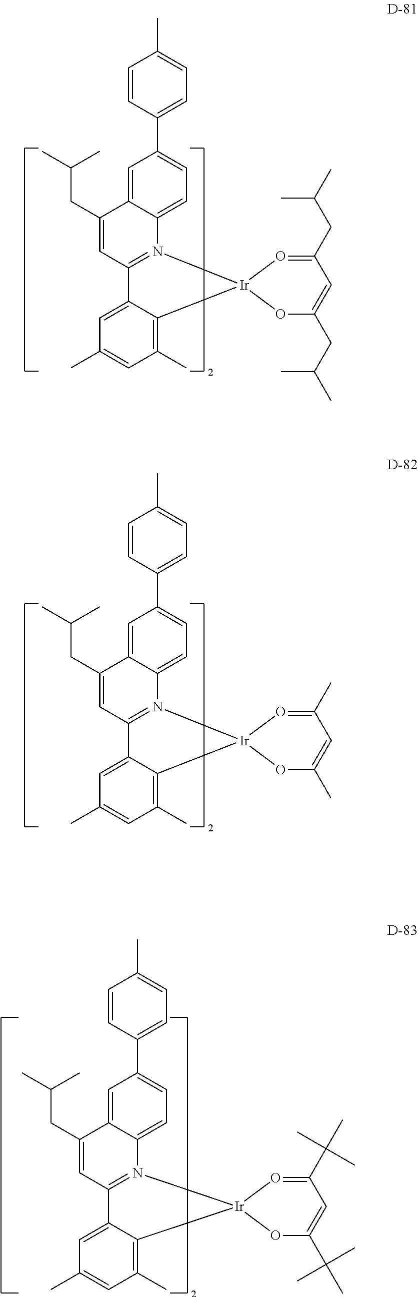

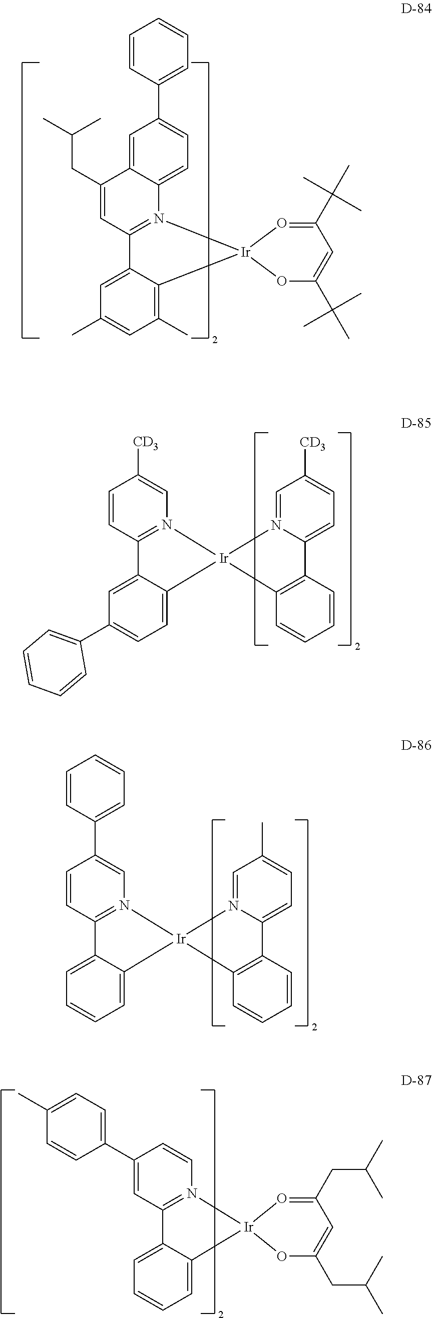

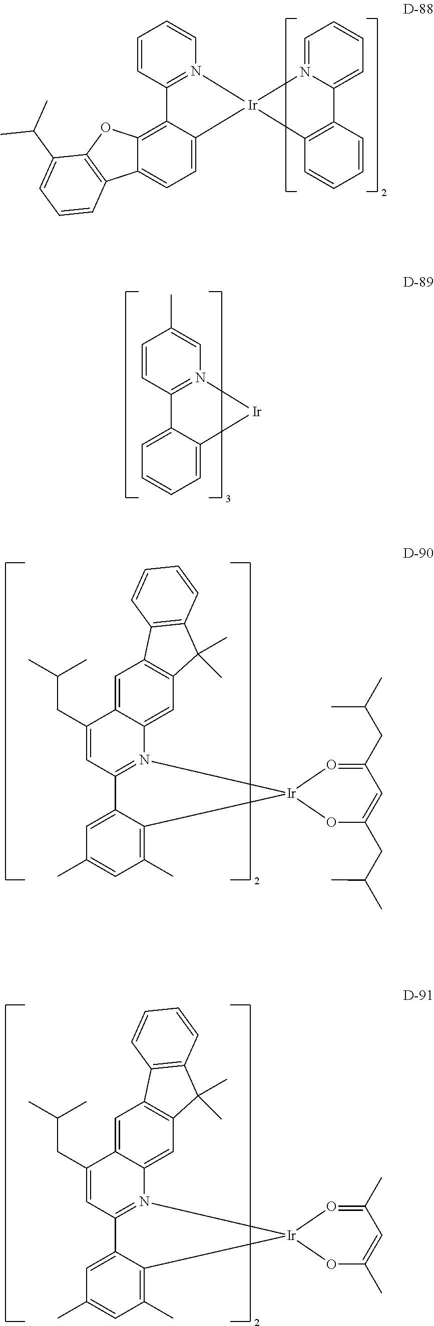

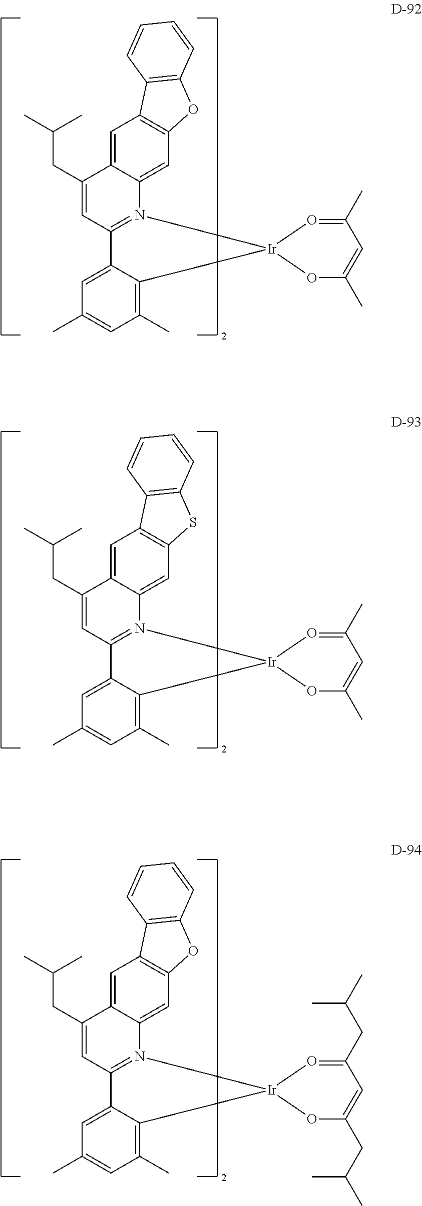

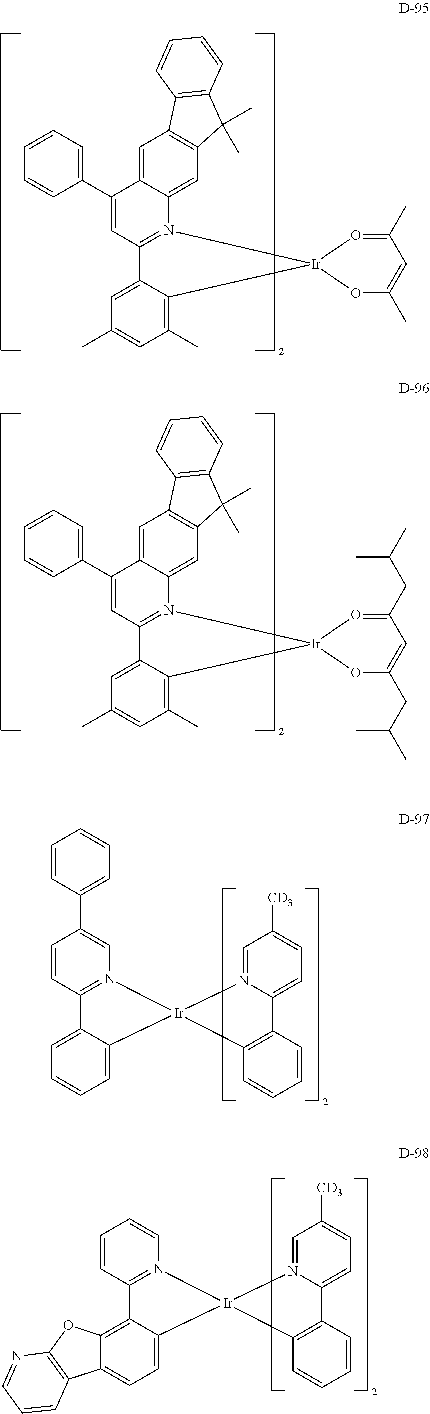

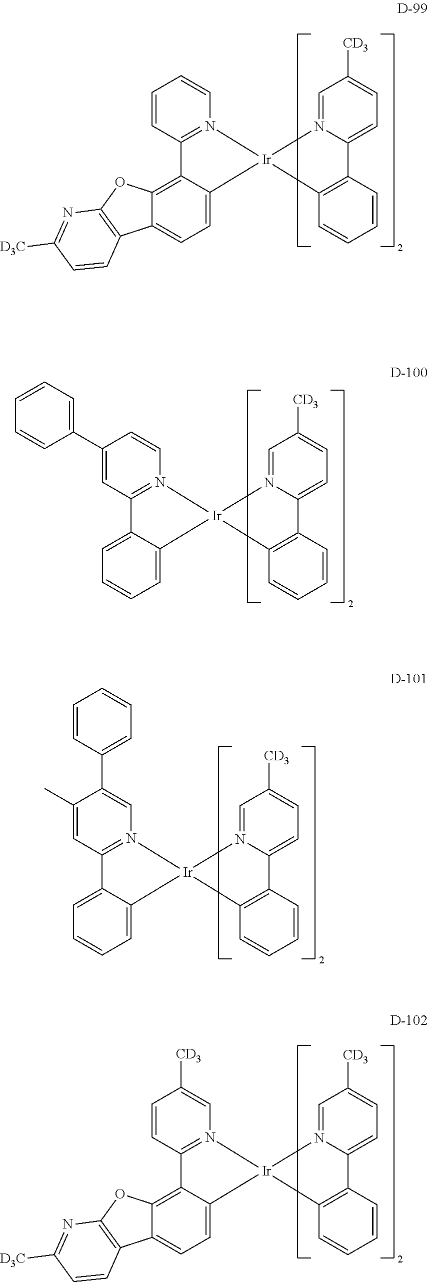

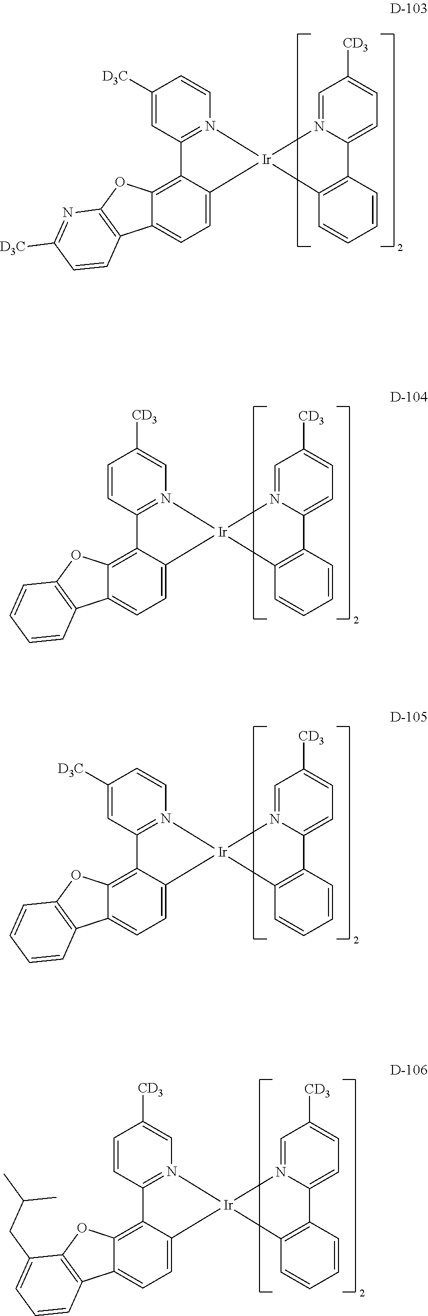

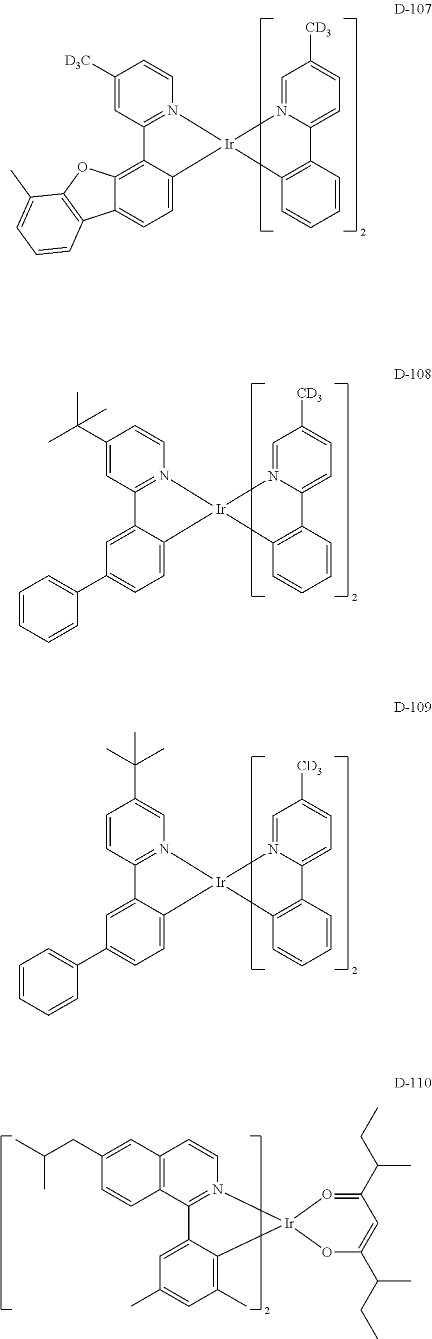

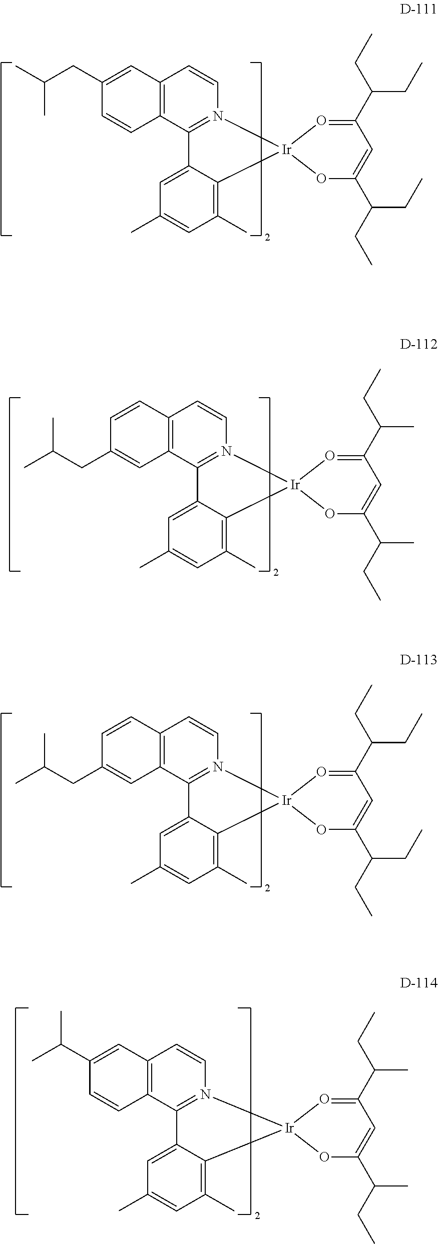

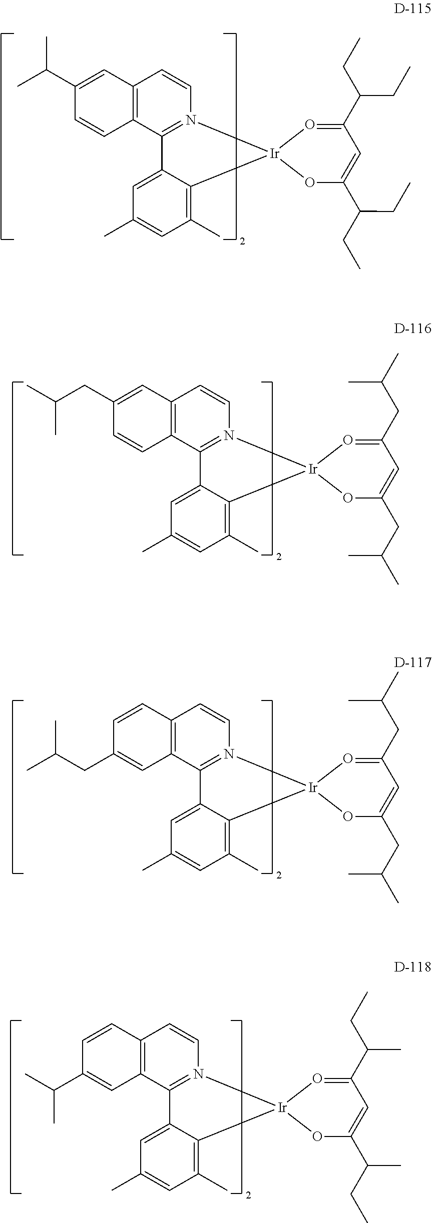

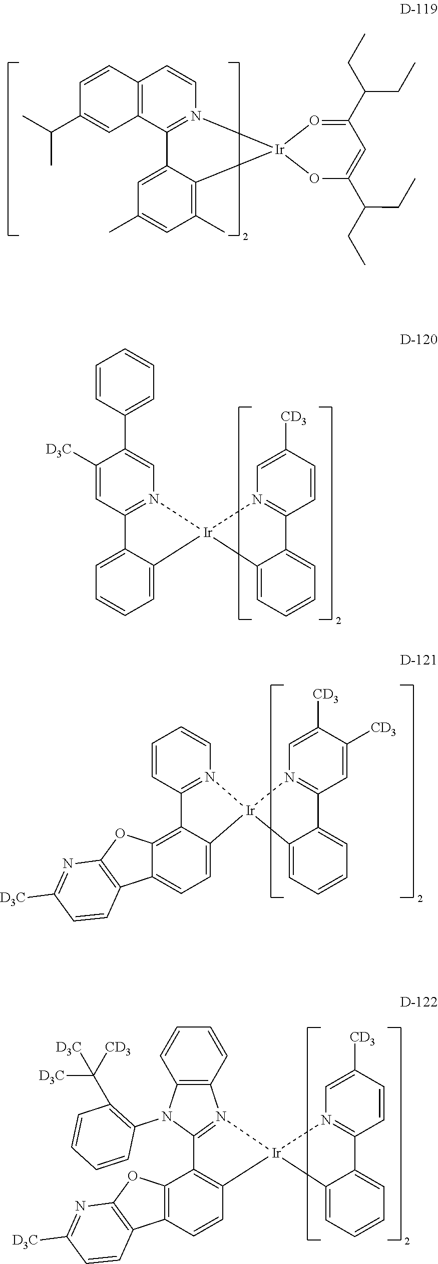

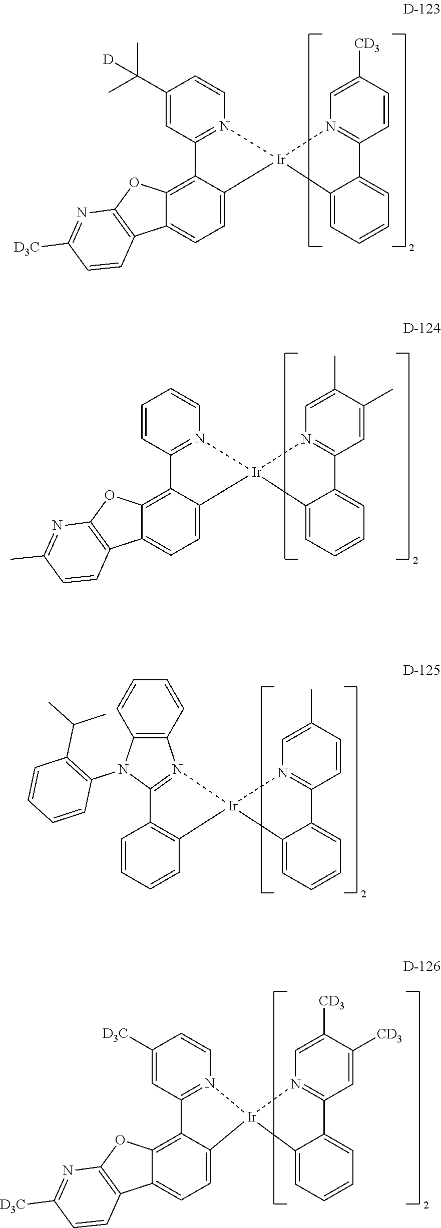

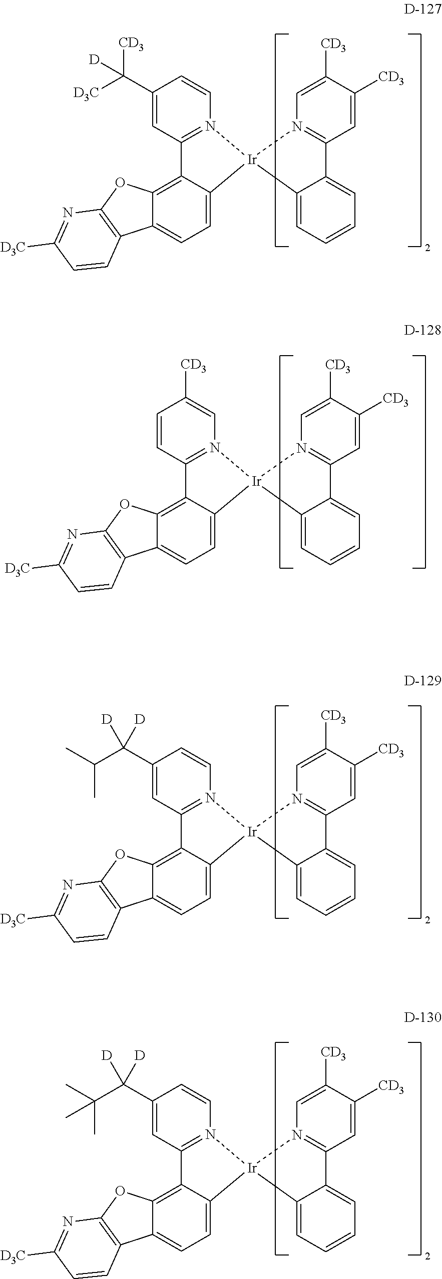

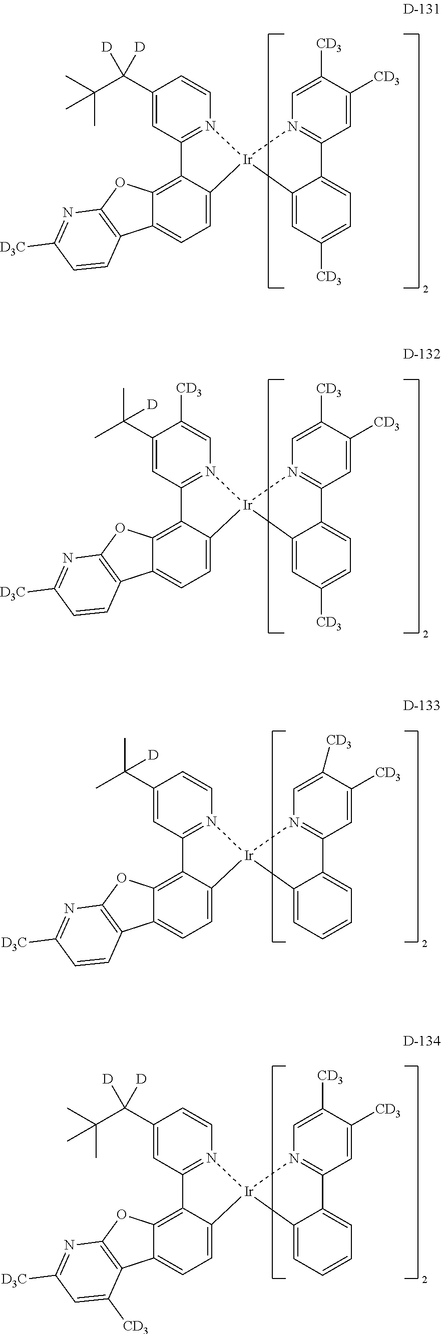

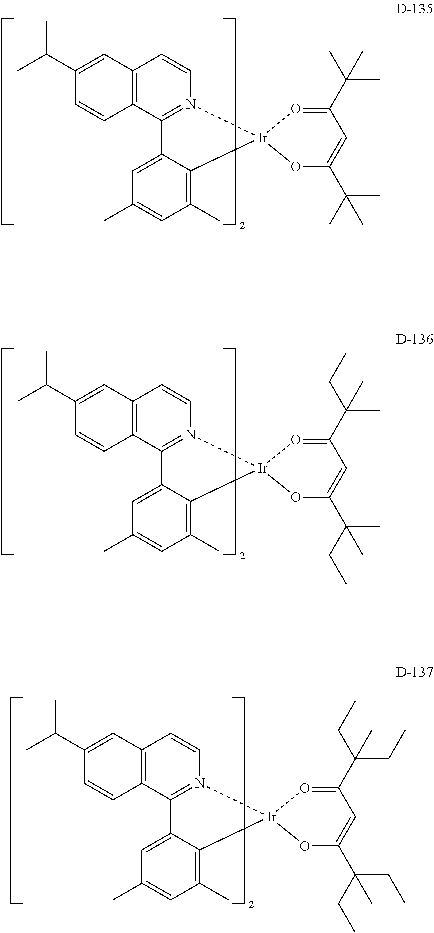

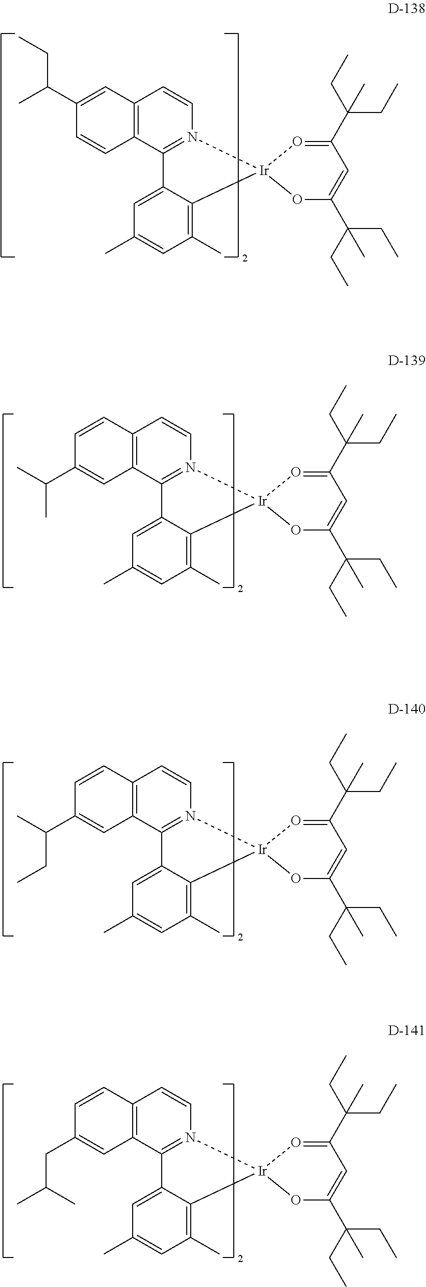

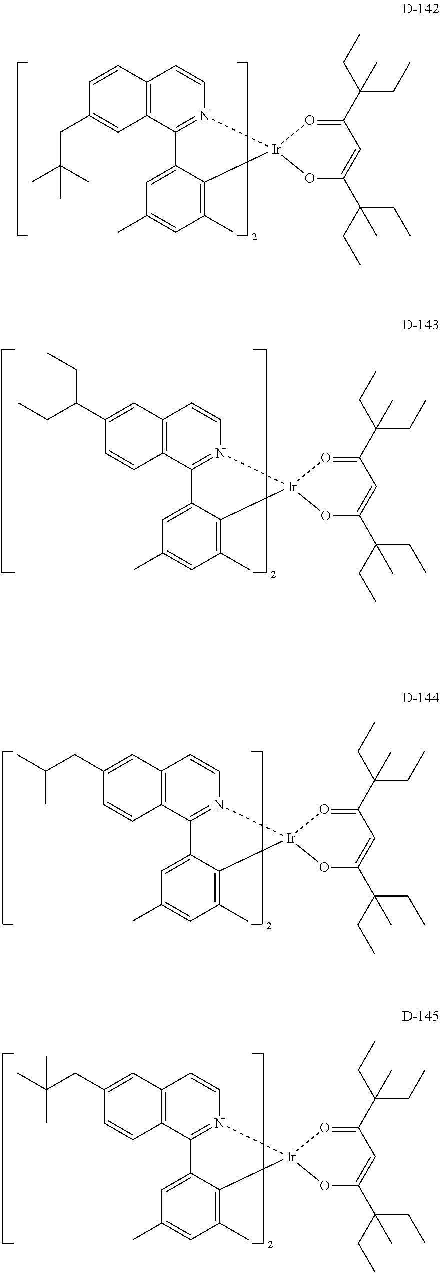

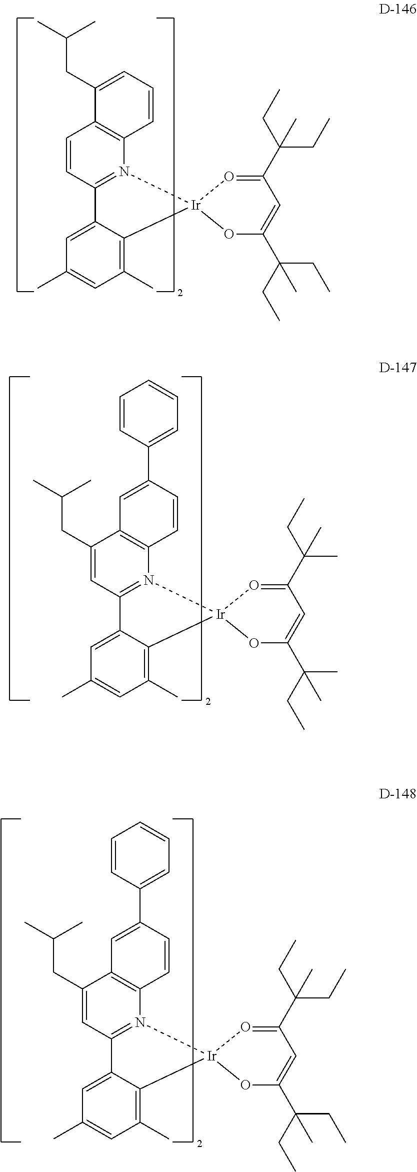

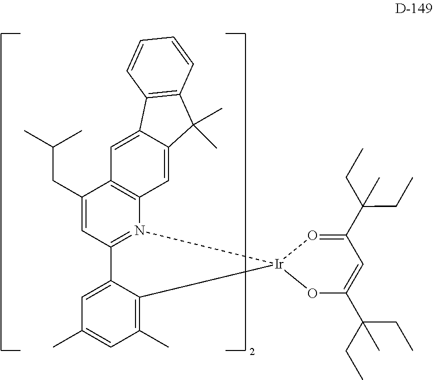

Hereinafter, an organic electroluminescent device to which the aforementioned organic electroluminescent compound and/or the aforementioned organic electroluminescent material is/are applied, will be described.

The organic electroluminescent device according to one embodiment includes a first electrode; a second electrode; and at least one organic layer interposed between the first electrode and the second electrode. The organic layer may comprise at least one layer(s) selected from a hole transport layer, a hole injection layer, an electron blocking layer, a hole auxiliary layer, a light-emitting auxiliary layer, a light-emitting layer, an electron transport layer, an electron injection layer, an interiayer, a hole blocking layer, and an electron auxiliary layer, and each layer can be further composed of several layers. Also, the organic layer may further comprise at least one compound(s) selected from the group consisting of an arylamine-based compound and a styrylarylamine-based compound, and further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The compound represented by formula 1 and/or the compound represented by formula 2 in the present disclosure may be included in one or more layers constituting an organic electroluminescent device. According to one embodiment, the organic layer includes a hole transport zone and/or an electron transport zone and/or a light-emitting layer including organic electroluminescent compound according to the present disclosure, for example, a hole transport layer and/or hole auxiliary layer and/or hole blocking layer and/or electron auxiliary layer and/or a light-emitting layer. For example, when the compound of formula 1 is included in the hole transport layer and/or the hole auxiliary layer and/or the hole blocking layer and/or the electron auxiliary layer and/or the light-emitting layer, the compound of formula 1 may be included as a hole transport material and/or a hole auxiliary material and/or a hole blocking material and/or an electron auxiliary material and/or a host material, respectively. The hole transport layer and/or hole auxiliary layer and/or hole blocking layer and/or electron auxiliary layer and/or light-emitting layer may include, for example, an organic electroluminescent compound of the present disclosure alone or a mixture of at least two of organic electroluminescent compounds and may further include conventional materials included in the organic electroluminescent material.

According to one embodiment, the hole transport layer may comprises at least one organic electroluminescent compound(s) represented by formula 1, for example, the hole transport layer may comprise at least one compound(s) of compounds C-1 to C-700 represented by formula 1. According to another embodiment, the hole transport layer may comprises at least one organic electroluminescent compound(s) represented by formula 2, for example, the hole transport layer may comprise at least one compound(s) of compounds C1-1 to C1-69 represented by formula 2. According to other embodiment, the hole transport layer may comprises at least one organic electroluminescent compound(s) represented by formula 3, for example, the hole transport layer may comprise at least one compound(s) of compounds C2-1 to C2-38 represented by formula 3.

The light-emitting layer according to one embodiment may include a plurality of host materials including at least one first host material represented by formula 1 and at least one second host material represented by formula 11. According to one embodiment, the light-emitting layer may comprise at least one of compounds C-1 to C-700 as the first host material represented by formula 1 and at least one of compounds H-1 to H-85 as the second host material represented by formula 11. According to another embodiment, the light-emitting layer may comprise an organic electroluminescent compound represented by formula 2. For example, the the light-emitting layer may comprise at least one compound(s) of compounds C1-1 to C1-69 represented by formula 2.

The hole blocking layer according to another embodiment may comprises at least one organic electroluminescent compound(s) represented by formula 1, for example, the hole blocking layer may comprise at least one of compounds C-1 to C-700 represented by formula 1.

An organic electroluminescent material according to one embodiment may be used as a material for an organic layer for a white organic light-emitting device. The white organic light-emitting device has suggested various structures such as a parallel side-by-side arrangement method, a stacking arrangement method, or color conversion material (CCM) method, etc., according to the arrangement of R (Red), G (Green). YG (yellowish green), or B (blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer also may be multi-layers, wherein each of the multi-layers may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X}(1≤X≤2), AlO_{X}(1≤X≤1.5), SiON, SiAlON, etc.; the halogenated metal includes LiF, MgF₂, CaF₂, a rare earth metal fluoride, etc.; and the metal oxide includes Cs₂O, Li₂O. MgO, SrO, BaO. CaO, etc.

Further, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the organic electroluminescent compound according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, the organic electroluminescent device of the present disclosure can be used for the manufacture of display devices such as smartphones, tablets, notebooks. PCs, TVs, or display devices for vehicles, or lighting devices such as outdoor or indoor lighting.

Hereinafter, the preparation method of compounds according to the present disclosure will be explained with reference to a representative compound or the intermediate compound in order to understand the present disclosure in detail.

### [Example 1] Synthesis of Compound C1-14

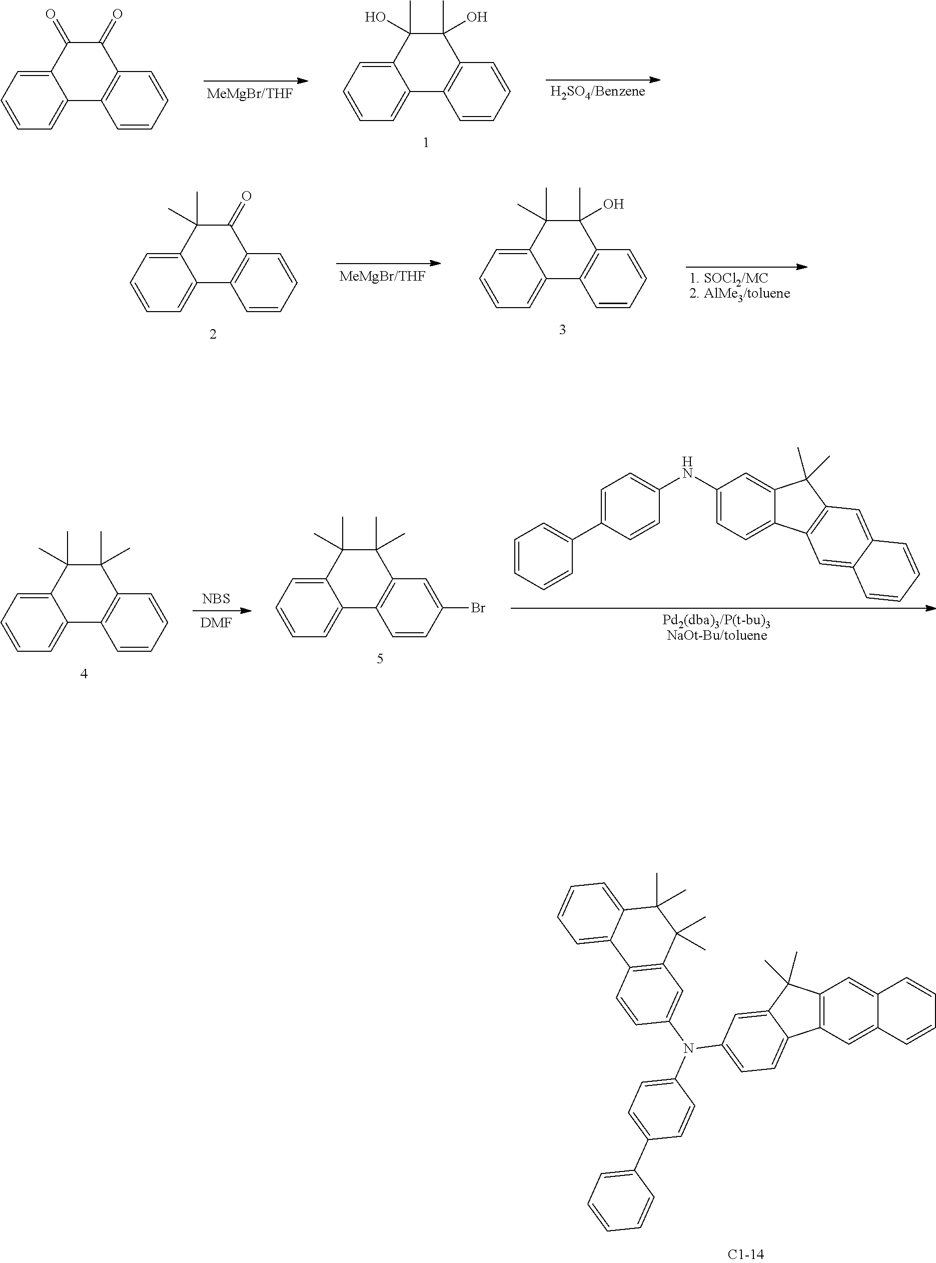

1) Synthesis of Compound 1

Phenanthrene-9,10-dione (100.0 g, 480 mmol) was added into a flask and dissolved in THF solution. Next, methylmagnesium bromide (MeMgBr) (3 M in THF) solution (480 mL, 1,440 mmol) was added dropwise at 0° C. under nitrogen charge, and then stirred for 2 hours. After completion of the reaction, the mixture was neutralized with aqueous ammonium chloride (NH₄Cl) solution, and then extracted with methyl chloride (MC), followed by drying with magnesium sulfate (MgSO₄). Next, it was separated by column chromatography, followed by adding methanol (MeOH) thereto. Thereafter, the resulting solid was filtered under reduced pressure to obtain compound 1 (43.0 g, yield: 36%).

2) Synthesis of Compound 2

Compound 1 (60.0 g, 250 mmol), H₂SO₄ (202 mL, 375 mmol), and 500 mL of benzene were added into a flask, and then stirred under reflux at 120° C. for 2 hours. After completion of the reaction, the mixture was neutralized with sodium bicarbonate (NaHCO₃), and then extracted with MC, followed by drying with MgSO₄. Next, it was separated by column chromatography, followed by adding MeOH thereto. Thereafter, the resulting solid was filtered under reduced pressure to obtain compound 2 (50.0 g, yield: 90%).

3) Synthesis of Compound 3

Compound 2 (20.0 g, 90.0 mmol) was added into a flask and dissolved in THF solution. Next, MeMgBr (3 M in THF) solution (45 mL, 135 mmol) was added dropwise at 0° C. under nitrogen charge, and then stirred for 2 hours. After completion of the reaction, the mixture was neutralized with isopropyl alcohol (IPA) and aqueous NH₄Cl solution, and then extracted with MC, followed by drying with MgSO₄. Next, it was separated by column chromatography, followed by adding MeOH thereto. Thererafter, the resulting solid was filtered under reduced pressure to obtain compound 3 (23.0 g, yield: 107%).

4) Synthesis of Compound 4

Compound 3 (18.6 g, 78 mmol) and 78 mL of thionyl chloride (1M in MC) solution were added into a flask, and then stirred for 2 hours at 0° C. After lowering the temperature to −78° C., 78 mL of trimethylaluminum (AlMe₃) (2M in Toluene) solution was added thereto, and then stirred for 3 hours, followed by reacting overnight at room temperature. After completion of the reaction, IPA and H₂O were added to quenching the solution, and then the layers were separated with MC. Next, it was separated by column chromatography, followed by adding MeOH thereto. Thereafter, the resulting solid was filtered under reduced pressure to obtain compound 4 (18.7 g, yield: 101%).

5) Synthesis of Compound 5

Compound 4 (19.2 g, 81 mmol) and 200 mL of DMF were added into a flask. N-bromosuccinimide (NBS) (26.0 g, 146 mmol) dissolved in 100 mL of DMF under nitrogen charge was added dropwise thereto, followed by reacting overnight, while stirring. After completion of the reaction, ethyl acetate (EA) and H₂O were added thereto, and then, the organic layer was separated to remove an organic solvent. Next, it was separated by column chromatography, followed by adding MeOH thereto. Thereafter, the resulting solid was filtered under reduced pressure to obtain compound 5 (23.2 g, yield: 90%).

6) Synthesis of Compound C1-14

Compound 5 (5.59 g, 24.8 mmol), N-([1,1′-biphenyl]-4-yl)-11,11-dimethyl-11H-benzo[b]fluoren-2-amine (10.2 g, 24.8 mmol), tris(dibenzylideneacetone)dipalladium (0)(Pd₂(dba)₃) (0.81 g, 0.89 mmol), tri-tert-butylphosphine (P(t-Bu)₃) (0.359 g, 1.77 mmol), sodium tert-butoxide (NaOt-Bu) (3.41 g, 35.5 mmol), and 60 mL of toluene were added into a flask, and stirred at 120° C. for 1.5 hours. After completion of the reaction, the organic solvent was removed, and then the resulting solid was separated by column chromatography. Next, MeOH was added thereto, and then the resulting solid was filtered under reduced pressure to obtain compound C1-14 (1.3 g, yield: 11%).

| | MW | color | M.P |
|---|---|---|---|
| C1-14 | 645.87 | white | 113° C. |

### [Example 2] Synthesis of Compound C-14

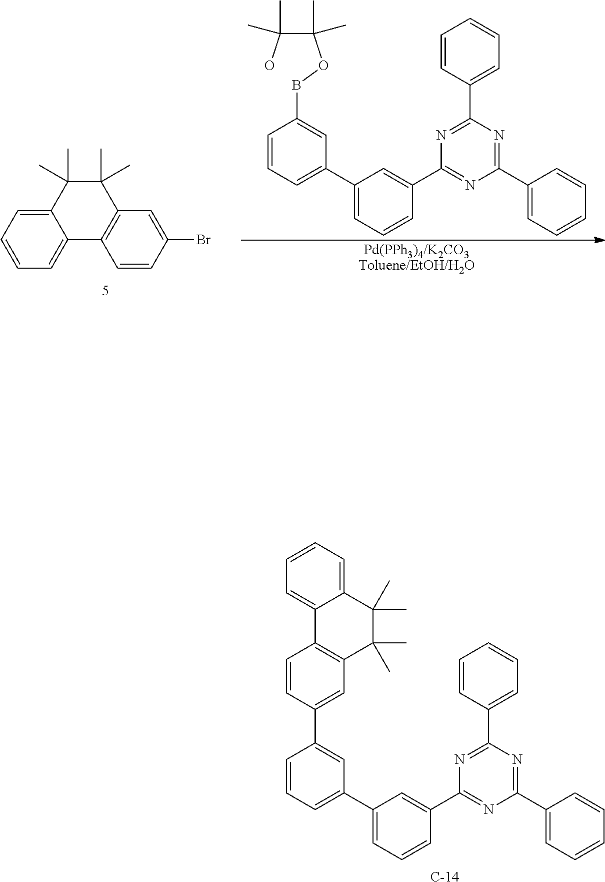

Compound 5 (6.0 g, 19.0 mmol), 2,4-diphenyl-6-(3′-(4,4,5,5-tetramethyl-1,3,2-dioxaborolan-2-yl)-[1,1′-biphenyl]-3-yl)-1,3,5-triazine (11.7 g, 22.8 mmol), Pd(PPh₃)₄ (1.10 g, 0.95 mmol), K₂CO₃ (7.9 g, 57 mmol), 50 mL of toluene, 25 mL of EtOH, and 25 mL of H₂O were added into a flask, and stirred under reflux at 140° C. After completion of the reaction, the organic solvent was removed, and the resulting solid was separated by column chromatography. Next, MeOH was added thereto, and then the resulting solid was filtered under reduced pressure to obtain compound C-14 (2.4 g, yield: 20.3%).

| | MW | color | M.P |
|---|---|---|---|
| C-14 | 619.81 | white | 126° C. |

### [Example 3] Synthesis of Compound C-578

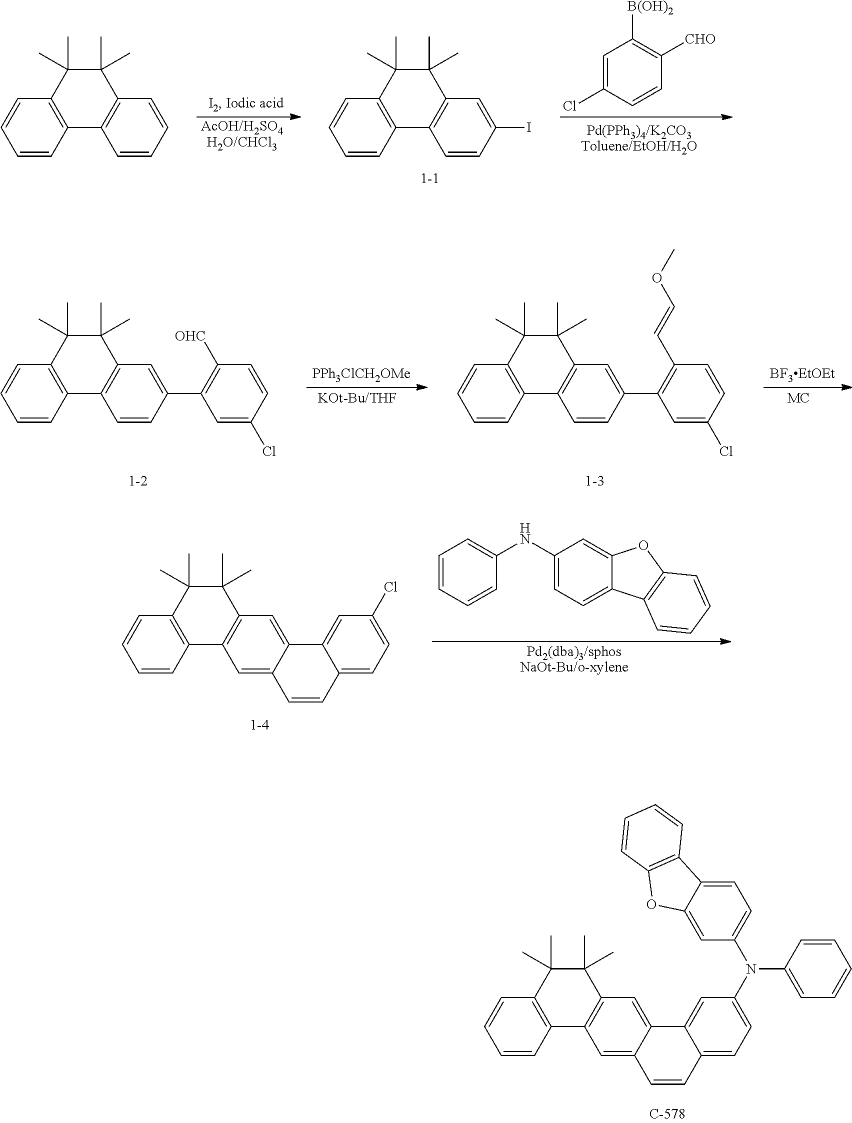

1) Synthesis of Compound 1-1

9,9,10,10-tetramethyl-9,10-dihydrophenanthrene (34.0 g, 144 mmol), iodine (I₂) (18.3 g, 71.9 mmol), iodic acid (12.7 g, 71.9 mmol), 280 mL of acetic acid (AcOH), 36 mL of H₂SO₄, 36 mL of water (H₂O), and 15 mL of CHCl₃ were added into a flask, and stirred at 65° C. After completion of the reaction, the solvent was removed, followed by separating with column chromatography. Next, MeOH was added thereto, and then the resulting solid was filtered under reduced pressure to obtain compound 1-1 (56.0 g, yield: 107%).

2) Synthesis of Compound 1-2

Compound 1-1 (35.0 g, 96.6 mmol), 5-chloro-2-formyl-phenyl)boronic acid (21.4 g, 116 mmol), Pd(PPh₃)₄ (5.58 g, 4.83 mmol), K₂CO₃ (33.4 g, 242 mmol), 300 mL of toluene, 100 mL of EtOH, and 100 mL of H₂O were added into a flask, and stirred at 140° C. After completion of the reaction, EA and H₂O were added to the reaction mixture to separate the layers, and then only an organic layer was separated. The solvent was removed by filtration under reduced pressure, followed by separating with column chromatography. Next, MeOH was added thereto, and then the resulting solid was filtered under reduced pressure to obtain compound 1-2 (36.0 g, yield: 99.4%).

3) Synthesis of Compound 1-3

Compound 1-2 (30.0 g, 80.0 mmol), chloro-(methoxymethyl)-trphenyl-lambda5-phosphane (38.4 g, 112 mmol), and 370 mL of THF were added into a flask and dissolved. Thereafter, 112 mL of KOt-Bu (1M in THF) solution was added dropwise thereto with stirring. After completion of the reaction, EA and H₂O were added to the reaction mixture to separate the layers, and then only an organic layer was separated. The solvent was removed by filtration under reduced pressure, followed by separating with column chromatography. Next, MeOH was added thereto, and then the resulting solid was filtered under reduced pressure to obtain compound 1-3 (20.0 g, yield: 62.0%).

4) Synthesis of Compound 1-4

Compound 1-3 (19.0 g, 47.2 mmol) and 250 mL of MC were added into a flask and dissolved. Thereafter, 17.8 mL of BF₃.EtOEt solution was added dropwise thereto, with stirring at 0° C. After completion of the reaction, MC and NaHCO₃ (aq) were added thereto to separate the layers, and then only an organic layer was separated. The solvent was removed by filtration under reduced pressure, followed by separating with column chromatography. Next, MeOH was added thereto, and then the resulting solid was filtered under reduced pressure to obtain compound 1-4 (16.0 g, yield: 91.5%).

5) Synthesis of Compound C-578

Compound 1-4 (6.0 g, 16.2 mmol), N-phenyldibenzofuran-3-amine (4.40 g, 17.0 mmol), Pd₂(dba)₃ (0.741 g, 0.809 mmol), sphos (0.664 g, 1.62 mmol), NaOt-Bu (3.11 g, 32.4 mmol), and 80 mL of o-xylene were added into a flask, and stirred under reflux at 180° C. After completion of the reaction, the solvent was removed by filtration under reduced pressure, followed by separating with column chromatography. Next, MeOH was added thereto, and then the resulting solid was filtered under reduced pressure to obtain compound C-578 (2.3 g, yield: 23.9%).

| | MW | color | M.P |
|---|---|---|---|
| C-578 | 593.27 | white | 188.4° C. |

### [Example 4] Synthesis of Compound C-470

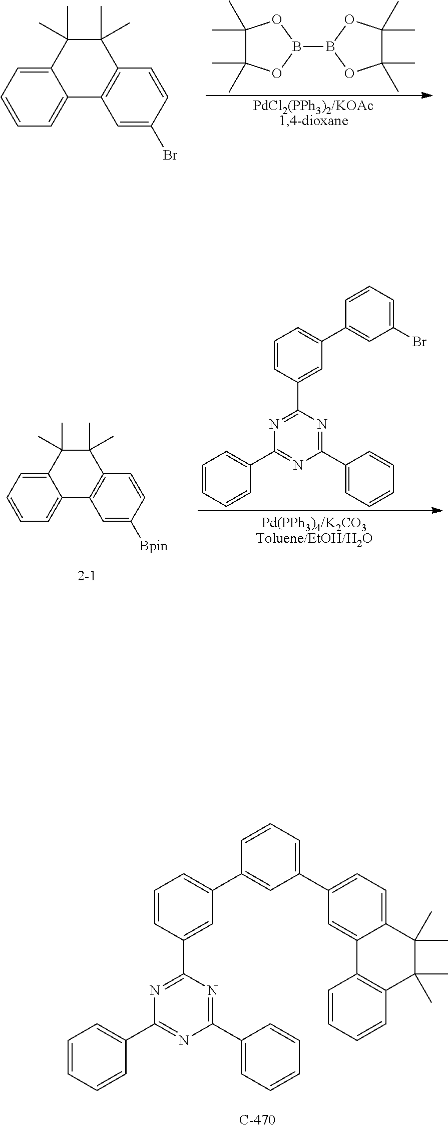

1) Synthesis of Compound 2-1

3-Bromo-9,9,10,10-tetramethyl-9,10-dihydrophenanthrene (30.0 g, 95.2 mmol), 4,4,4′,4′,5,5,5′,5′-octamethyl-2,2′-bi(1,3,2-dioxaborolane) (29.0 g, 114.1 mmol), PdCl₂(PPh₃)₂ (3.34 g, 4.76 mmol), KOAc (23.3 g, 237.9 mmol), and 500 mL of 1,4-dioxane were added into a flask, and stirred at 140° C. for 3 hours. After completion of the reaction, the organic solvent was removed, and then the resulting solid was separated by column chromatography. Next, MeOH was added thereto, and then the resulting solid was filtered under reduced pressure to obtain compound 2-1 (31 g, yield: 90%).

2) Synthesis of Compound C-470

Compound 2-1 (6.0 g, 16.6 mmol), 2-(3′-bromo-[1,1′-biphenyl]-3-yl)-4,6-diphenyl-1,3,5-triazine (7.94 g, 18.2 mmol), Pd(PPh₃)₄ (0.960 g, 0.83 mmol), K₂CO₃ (6.88 g, 49.8 mmol), 40 mL of Toluene, 20 mL of EtOH, and 20 mL of H₂O were added into a flask, and then stirred at 140° C. for 2 hours. After completion of the reaction, the organic solvent was removed, and then the resulting solid was separated by column chromatography. Next, MeOH was added thereto, and then the resulting solid was filtered under reduced pressure to obtain compound C-470 4.5 yield: 44%).

| | MW | Color | M.P |
|---|---|---|---|
| C-470 | 619.8 | White | 147.7° C. |

### [Example 5] Synthesis of Compound C-77

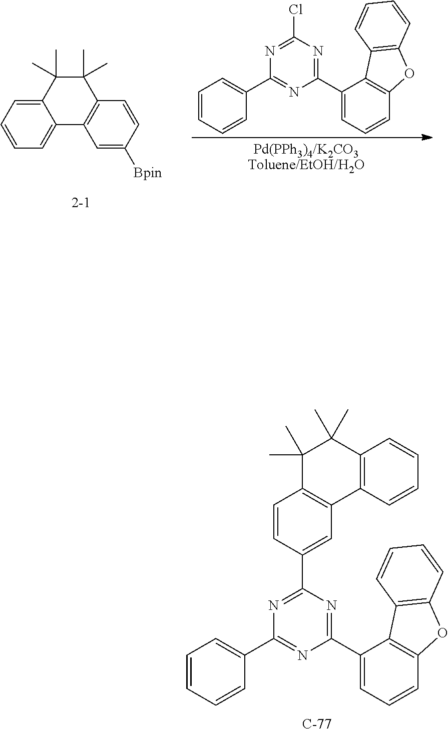

Compound 2-1 (4.5 g, 12.4 mmol), 2-chloro-4-(dibenzo[b,d]furan-1-yl)-6-phenyl-1,3,5-triazine (4.65 g, 13.0 mmol), Pd(PPh₃)₄ (0.716 g, 0.62 mmol), K₂CO₃ (6.88 g, 31.0 mmol), 30 mL of Toluene, 15 mL of EtOH, and 15 mL of H₂O were added into a flask, and stirred at 140° C. for 2 hours. After completion of the reaction, the organic solvent was removed, and then the resulting solid was separated by column chromatography. Next, MeOH was added thereto, and then the resulting solid was filtered under reduced pressure to obtain compound C-77 (4.5 g, yield: 44%).

| | MW | color | M.P |
|---|---|---|---|
| C-77 | 557.68 | white | 198.1° C. |

### [Example 6] Synthesis of Compound C-652

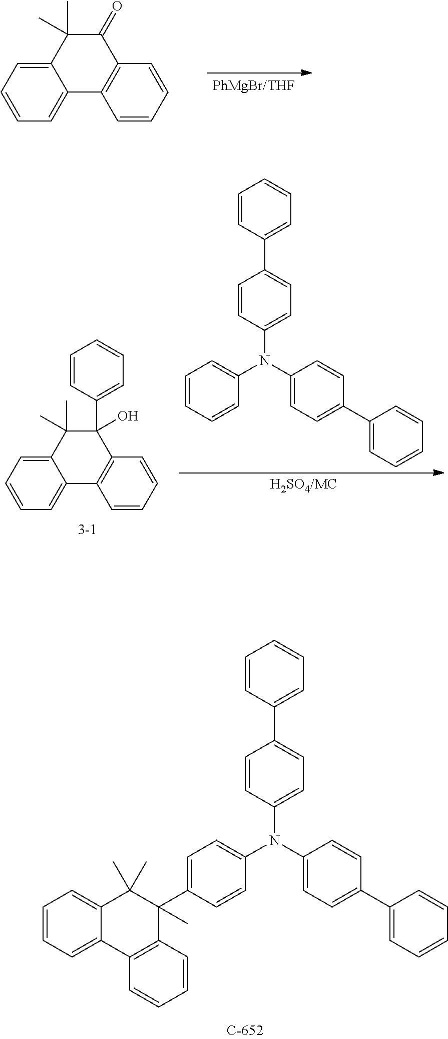 [SMILES: CC1(C)C(=O)c2ccccc2-c2ccccc21 ; "PhMgBr/THF" ; CC1(C)c2ccccc2-c2ccccc2C1(O)c1ccccc1 ; c1ccc(-c2ccc(N(c3ccccc3)c3ccc(-c4ccccc4)cc3)cc2)cc1 ; "H2SO4/MC" ; CC1(C)c2ccccc2-c2ccccc2C1(C)c1ccc(N(c2ccc(-c3ccccc3)cc2)c2ccc(-c3ccccc3)cc2)cc1]

1) Synthesis of Compound 3-1

10,10-dimethylphenanthren-9 (10H)-one (10.0 g, 45.0 mmol) was added into a flask, and then dissolved in THF solution. Next, phenylmagnessium bromide (PhMgBr) (3 M in THF) solution (22.5 mL, 67.5 mmol) was added dropwise under nitrogen charge at 0° C., and then stirred for 2 hours. After completion of the reaction, the mixture was was neutralized with aqueous NH₄Cl solution, and then extracted with MC, followed by drying with MgSO₄. Next, it was separated by column chromatography. Thereafter, MeOH was added thereto, and then the resulting solid was filtered under reduced pressure to obtain compound 3-1 (12.5 g, yield: 92%).

2) Synthesis of Compound C-652

Compound 3-1 (12.4 g, 41.3 mmol), N-([1,1′-biphenyl]-4-yl)-N-phenyl-[1,1′-biphenyl]-4-amine (65.6 g, 165 mmol), and 200 mL of MC were added into a flask, and then stirred at 0° C. 6.7 mL of H₂SO₄ was added dropwise, followed by reacting for one (1) day. After completion of the reaction, the mixture was was neutralized with K₂CO₃, and then extracted with MC, followed by drying with MgSO₄. Next, it was separated by column chromatography. Thereafter, MeOH was added thereto, and then the resulting solid was filtered under reduced pressure to obtain compound C-652 (8.6 g, yield: 31%).

| | MW | color | M.P |
|---|---|---|---|
| C-652 | 679.8 | white | 225.5° C. |

### [Example 7] Synthesis of Compound C-469

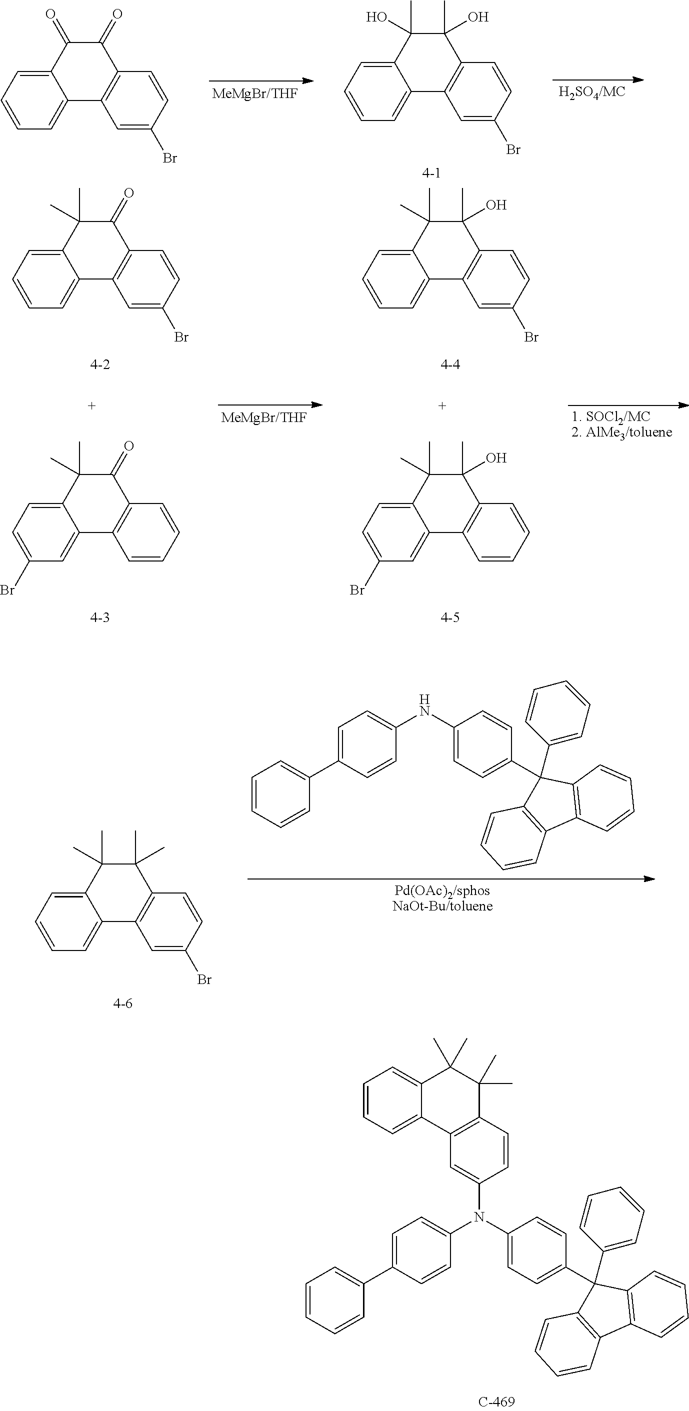

1) Synthesis of Compound 4-1

3-Bromophenanthrene-9,10-dione (60.0 g, 209 mmol) was added into a flask, and then dissolved in THF solution (1 L). Next, MeMgBr (3 M in THF) solution (209 mL, 627 mmol) was added dropwise under nitrogen charge at 0° C., and then stirred for 1 hour. After completion of the reaction, MeMgBr was quenched with IPA and MeOH and H₂O, and then neutralized with aqueous NH₄Cl solution. Next, the organic layer was extracted with EA, followed by drying with MgSO₄. Then, it was separated by Celite filter, and then MeOH was added thereto. Thereafter, the resulting solid was filtered under reduced pressure to obtain compound 4-1 (74.0 g, yield: 110%).

2) Synthesis of Compounds 4-2 and 4-3

Compound 4-1 (74.0 g, 232 mmol), H₂SO₄ (18.9 mL, 348 mmol), and 1,000 mL of MC were added into a flask, and then stirred under reflux at 80° C. for 1 hour. After completion of the reaction, H₂O was added the mixture to dilute H₂SO₄, and then the mixture neutralized with NaHCO₃. Next, it was extracted with MC, followed by drying with MgSO₄. Thereafter, it was separated by column chromatography, and then MeOH was added thereto. Thereafter, the resulting solid was filtered under reduced pressure to obtain compounds 4-2 and 4-3 (60.0 g, yield: 85%).

3) Synthesis of Compounds 4-4 and 4-5

Compounds 4-2 and 4-3 (60.0 g, 199 mmol) were added into a flask, and then dissolved in THF solution (1 L). Thereafter, MeMgBr (3 M in THF) solution (99.6 mL, 299 mmol) was added dropwise under nitrogen charge at 0° C., and stirred for 3 hours. After completion of the reaction, the mixture was neutralized with IPA and aqueous NH₄Cl solution, and then extracted with MC, followed by drying with MgSO₄. Thereafter, it was separated by column chromatography, and then MeOH was added thereto. Next, the resulting solid was filtered under reduced pressure to obtain compounds 4-4 and 4-5 (63.2 g, yield: 100%).

4) Synthesis of Compound 4-6

Compounds 4-4 and 4-5 (63.2 g, 199.2 mmol) and 183 mL of thionyl chloride (SOCl₂) (1M in MC) solution were added into a flask, and then stirred for 2 hours at 0° C. After lowering the temperature to −78° C., 183 mL of AIMe₃ (2M in Toluene) solution was added thereto, and then stirred for 3 hours, followed by reacting overnight at room temperature.

After completion of the reaction, IPA and H₂O were added to quenching the solution, and then the layers were separated with MC. Next, it was separated by column chromatography, and then MeOH was added thereto. Thereafter, the resulting solid was filtered under reduced pressure to obtain compound 4-6 (59.0 g, yield: 94%).

5) Synthesis of Compound C-469

Compound 4-6 (5.0 g, 15.7 mmol), N-([1,1′-biphenyl]-4-yl)-11,11-dimethyl-11H-benzo[b]fluoren-2-amine (8.45 g, 17.4 mmol), Pd₂(dba)₃ (0.719 g, 0.785 mmol), P(t-Bu)₃ (0.318 g, 1.57 mmol), NaOt-Bu (3.02 g, 31.4 mmol), and 60 mL of toluene were added into a flask, and then stirred at 130° C. for 1 hour. After completion of the reaction, the organic solvent was removed, and then the resulting solid was separated by column chromatography. Thereafter, MeOH was added thereto, and then the resulting solid was filtered under reduced pressure to obtain compound C-469 (2.1 g, yield: 19%).

| | MW | color | M.P |
|---|---|---|---|
| C-469 | 719.97 | white | 240° C. |

### [Example 8] Synthesis of Compound C-317

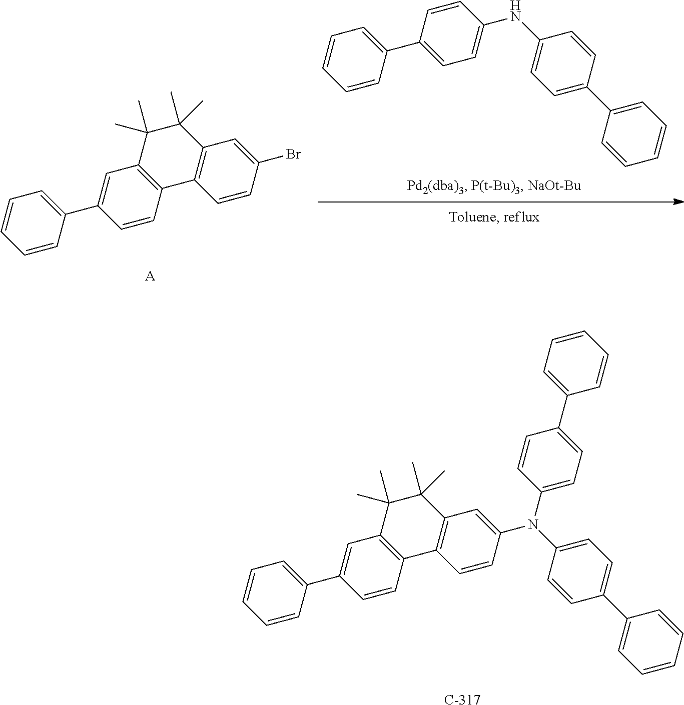

Compound A (2.6 g, 6.64 mmol), di([1,1′-biphenyl]-4-yl)amine (2.1 g, 6.64 mmol), Pd₂(dba)₃ (0.3 g, 0.33 mmol), P(t-Bu)₃ (0.3 mL, 0.66 mmol), NaOt-Bu (1.0 g, 9.96 mmol), and 33 mL of toluene were added to a reaction vessel, and stirred under reflux for 1 hour. After completion of the reaction, the reaction mixture was cooled to room temperature, and then the solid was filtrated, followed by washing with ethyl acetate. Next, the filtrate was distilled under reduced pressure and then purified by column chromatography to obtain compound C-317 (2.5 g, yield: 59%).

| | MW | M.P |
|---|---|---|
| C-317 | 631.85 | 235° C. |

### [Example 9] Synthesis of Compound C-400

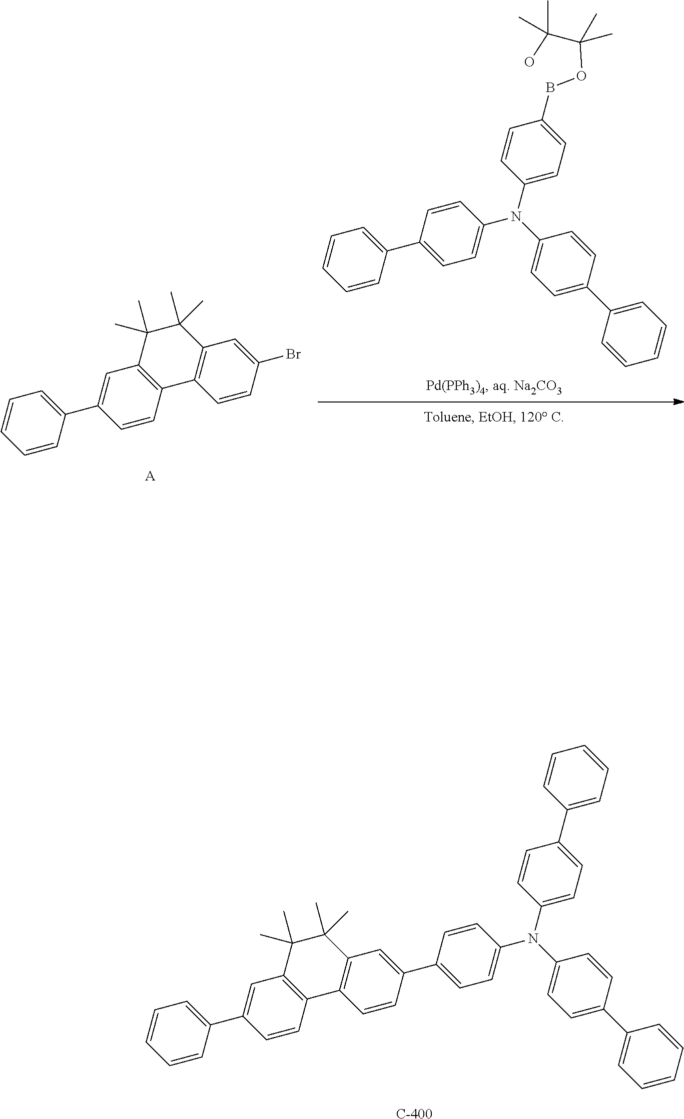

Compound A (3.5 g, 8.94 mmol), N-([1,1′-biphenyl]-4-yl)-N-(4-(4,4,5,5-tetramethyl-1,3,2-dioxaboran-2-yl)phenyl)-[1,1′-biphenyl]-4-amine (4.7 g, 8.94 mmol), Pd(PPh₃)₄ (0.5 g, 0.45 mmol), Na₂CO₃ (2.4 g, 22.35 mmol), 45 mL of Toluene, 11 mL of ethanol, and 11 mL of H₂O were added to a reaction vessel, and then stirred at 120° C. for 4 hours. After completion of the reaction, the mixture was washed with distilled water, and then the organic layer was extracted with ethyl acetate. Thereafter, the extracted organic layer was dried with magnesium sulfate. Next, the solvent was removed by a rotary evaporator and purified by column chromatography to obtain compound C-400 (2.0 g, yield: 32%).

| | MW | M.P |
|---|---|---|
| C-400 | 707.94 | 296° C. |

### [Example 10] Synthesis of Compound C2-31

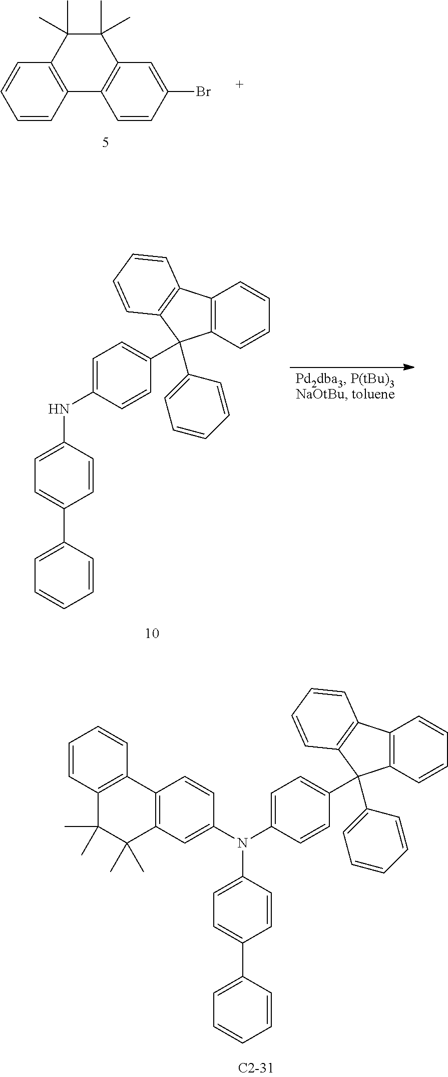 [SMILES: CC1(C)c2ccccc2-c2ccc(Br)cc2C1(C)C ; c1ccc(-c2ccc(Nc3ccc(C4(c5ccccc5)c5ccccc5-c5ccccc54)cc3)cc2)cc1 ; "Pd2dba3, P(tBu)3NaOtBu, toluene" ; CC1(C)c2ccccc2-c2ccc(N(c3ccc(-c4ccccc4)cc3)c3ccc(C4(c5ccccc5)c5ccccc5-c5ccccc54)cc3)cc2C1(C)C]

Compound 5 (6.5 g, 20.6 mmol), compound 10 (10.0 g, 20.6 mmol), Pd₂(dba)₃ (943 mg, 1.03 mmol), P(t-Bu)₃ (1.0 mL, 2.06 mmol, 50% Toluene solution), NaOt-Bu (3.0 g, 30.9 mmol), and 103 mL of toluene were added into a flask, and refluxed for 3 hours. After completion of the reaction, the reaction mixture was cooled to room temperature, and then the solvent was removed by a rotary evaporator and purified by column chromatography to obtain compound C2-31 (5.3 g, yield: 36% as a white solid.

| | MW | M.P |
|---|---|---|
| C2-31 | 719.97 | 140° C. |

### [Example 11] Synthesis of Compound C2-8

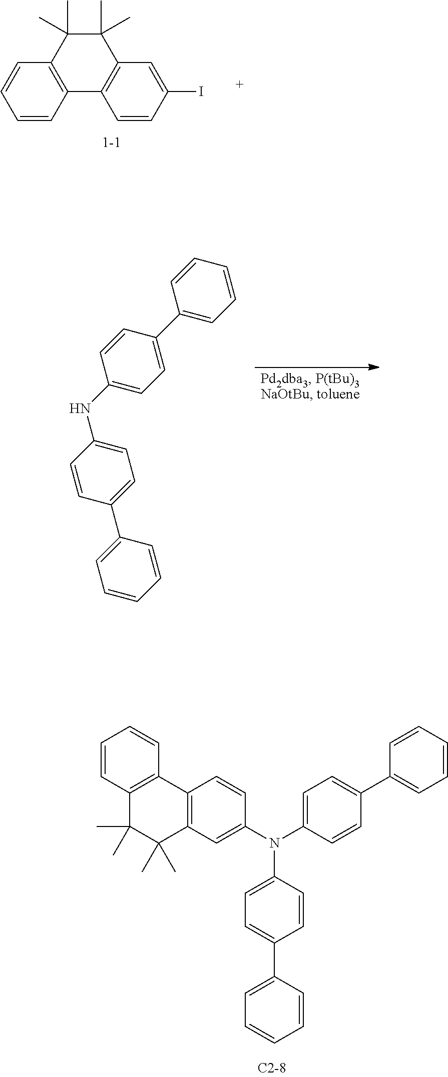 [SMILES: CC1(C)c2ccccc2-c2ccc(I)cc2C1(C)C ; c1ccc(-c2ccc(Nc3ccc(-c4ccccc4)cc3)cc2)cc1 ; "Pd2dba3, P(tBu)3NaOtBu, toluene" ; CC1(C)c2ccccc2-c2ccc(N(c3ccc(-c4ccccc4)cc3)c3ccc(-c4ccccc4)cc3)cc2C1(C)C]

Compound 1-1 (9.0 g, 24.8 mmol), di([1,1′-biphenyl]-4-yl)amine (9.6 g, 29.8 mmol), Pd₂(dba)₃ (1.1 g, 1.24 mmol), P(t-Bu)ₐ (1.2 mL, 2.48 mmol, 50% Toluene solution), NaOt-Bu (4.8 g, 49.6 mmol), and 130 mL of toluene were added into a flask, and refluxed for 4 hours. After completion of the reaction, the reaction mixture was cooled to room temperature, and then the solvent was removed by a rotary evaporator and purified by column chromatography to obtain compound C2-8 (4.1 g, yield: 30%) as a white solid.

| | MW | M.P |
|---|---|---|
| C2-8 | 555.77 | 154° C. |

### [Example 12] Synthesis of Compound C2-32

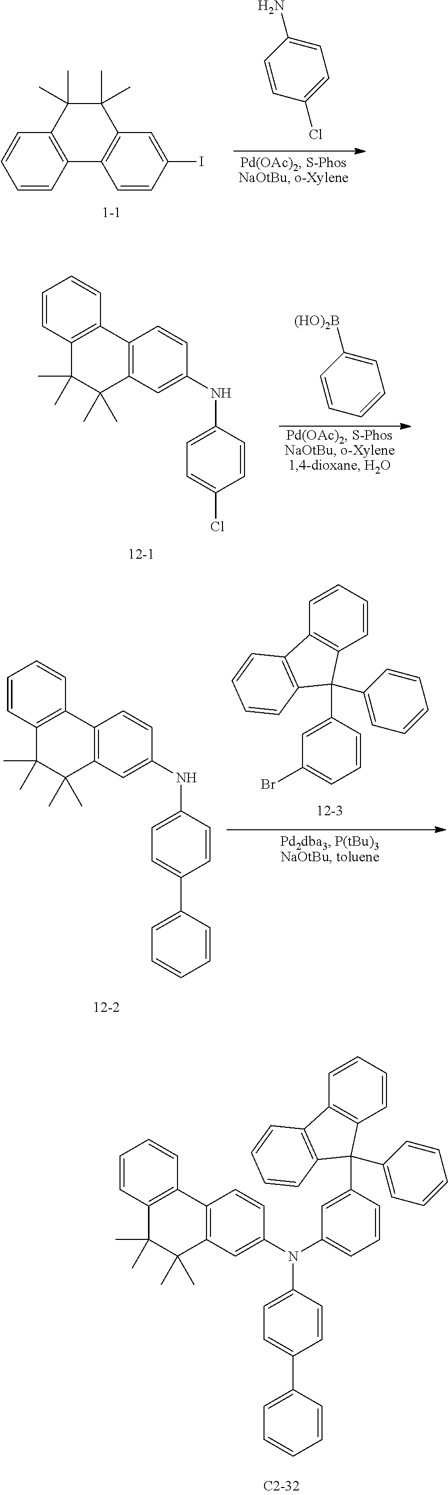

1) Synthesis of Compound 12-1

Compound 1-1 (30.0 g, 82.8 mmol), 4-chloroaniline (21.7 g, 169.8 mmol), palladium (II) acetate (Pd(OAC)₂) (1.3 g, 5.68 mmol), S-Phos (4.6 g, 11.3 mmol), NaOt-Bu (16.3 g, 169.8 mmol), and 566 mL of o-xylene were added to a flask, and refluxed for 3 hours. After completion of the reaction, the reaction mixture was cooled to room temperature, and then the solvent was removed by a rotary evaporator and purified by column chromatography to obtain compound 12-1 (18 g, yield: 60%).

2) Synthesis of Compound 12-2

Compound 12-1 (18.0 g, 49.7 mmol), phenyl boronic acid (13.2 g, 74.6 mmol), Pd(OAC)₂ (559 mg, 2.49 mmol), S-Phos (2.0 g, 4.97 mmol), NaOt-Bu (12 g, 124.4 mmol), 250 mL of o-xylene, 60 mL of 1,4-dioxane, and 60 mL of distilled water were added to a flask, and refluxed for 4 hours. After completion of the reaction, the reaction mixture was cooled to room temperature, and then the solvent was removed by a rotary evaporator and purified by column chromatography to obtain compound 12-2 (18.1 g, yield: 90%).

3) Synthesis of Compound C2-32

Compound 12-2 (10.2 g, 25.2 mmol), compound 12-3 (10.0 g, 25.2 mmol), Pd₂(dba)₃ (1.2 g, 1.26 mmol), P(t-Bu)₃ (1.24 mL, 2.52 mmol, 50% Toluene solution), NaOt-Bu (3.6 g, 37.8 mmol), and 126 mL of Toluene were added into a flask, and then refluxed for 4 hours. After completion of the reaction, the reaction mixture was cooled to room temperature, and then the solvent was removed by a rotary evaporator and purified by column chromatography to obtain compound C2-32 (5.9 g, yield: 33%) as a white solid.

| | MW | M.P |
|---|---|---|
| C2-32 | 719.97 | 126° C. |

### [Example 13] Synthesis of Compound C-696

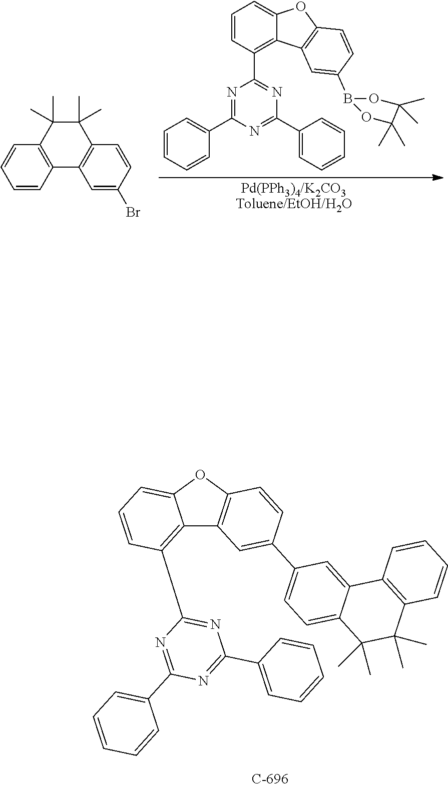

3-Bromo-9,9,10,10-tetramethyl-9,10-dihydrophenanthrene (5.5 g, 10.4 mmol), 2,4-diphenyl-6-(8-(4,4,5,5-tetramethyl-1,3,2-dioxaborolan-2-yl)dibenzo[b,d]furan-1-yl)-1,3,5-triazine (4.3 g, 13.5 mmol), Pd(PPh₃)₄ (0.6 g, 0.52 mmol), K₂CO₃ (2.8 g, 20.8 mmol), 100 mL of toluene, 20 mL of H₂O, and 20 mL of EtOH were added into a flask, and stirred at 150° C. After completion of the reaction, EA and H₂O were added to the reaction mixture to separate the layers, and then only an organic layer was separated. Thereafter, the solvent was removed by filtration under reduced pressure, followed by separating with column chromatography. Next, MeOH was added thereto, and then the resulting solid was filtered under reduced pressure to obtain compound C-696 (5.4 g, yield: 83%).

| | MW | color | M.P |
|---|---|---|---|
| C-696 | 633.8 | white | 129° C. |

### [Example 14] Synthesis of Compound C-697

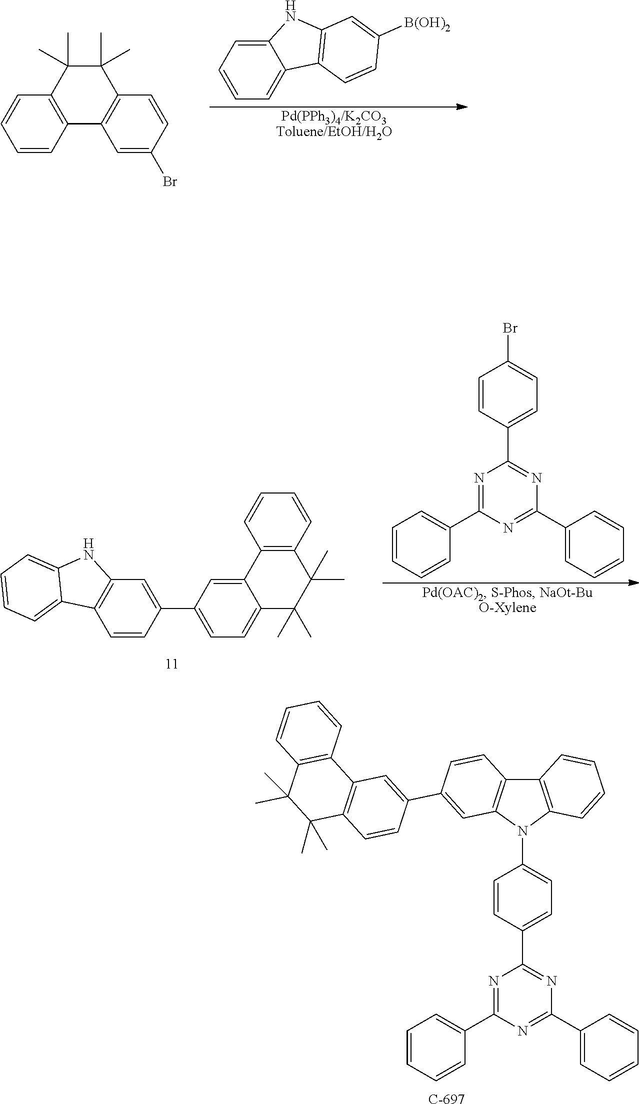

1) Synthesis of Compound 11

3-bromo-9,9,10,10-tetramethyl-9,10-dihydrophenanthrene (13.3 g, 42.1 mmol), (9H-carbazol-2-yl)boronic acid (13.3 g, 63.1 mmol), Pd(PPh₃)₄ (2.43 g, 2.1 mmol), K₂CO₃ (11.6 g, 84.2 mmol), 210 mL of toluene, 40 mL of H₂O, and 20 mL of EtOH were added into a flask, and stirred at 150° C. After completion of the reaction, only the organic layer was separated by adding EA and H₂O, and then the solvent was removed by filtration under reduced pressure. Thereafter, it was separated by column chromatography, and then MeOH was added thereto. Next, the resulting solid was filtered under reduced pressure to obtain compound 11 (6.9 g, yield: 40.8%).

2) Synthesis of Compound C-697

Compound 11 (6.9 g (15.9 mmol), 2-(4-bromophenyl)-4,6-diphenyl-1,3,5-triazine (6.8 g, 17.5 mmol). Pd(OAC)₂ (0.18 g, 0.8 mmol), S-Phos (0.65 g, 1.59 mmol), NaOt-Bu (3.0 g, 31.8 mmol) and 160 mL of o-xylene were added into a flask, and then stirred at 180° C. After completion of the reaction, only the organic layer was separated by adding EA and H₂O, and then the solvent was removed by filtration under reduced pressure. Thereafter, it was separated by column chromatography, and then MeOH was added thereto. Next, the resulting solid was filtered under reduced pressure to obtain compound C-697 (4.8 g. yield: 42.8%).

| | MW | color | M.P |
|---|---|---|---|
| C-697 | 708.9 | white | 300° C. |

### [Example 15] Synthesis of Compound C-572

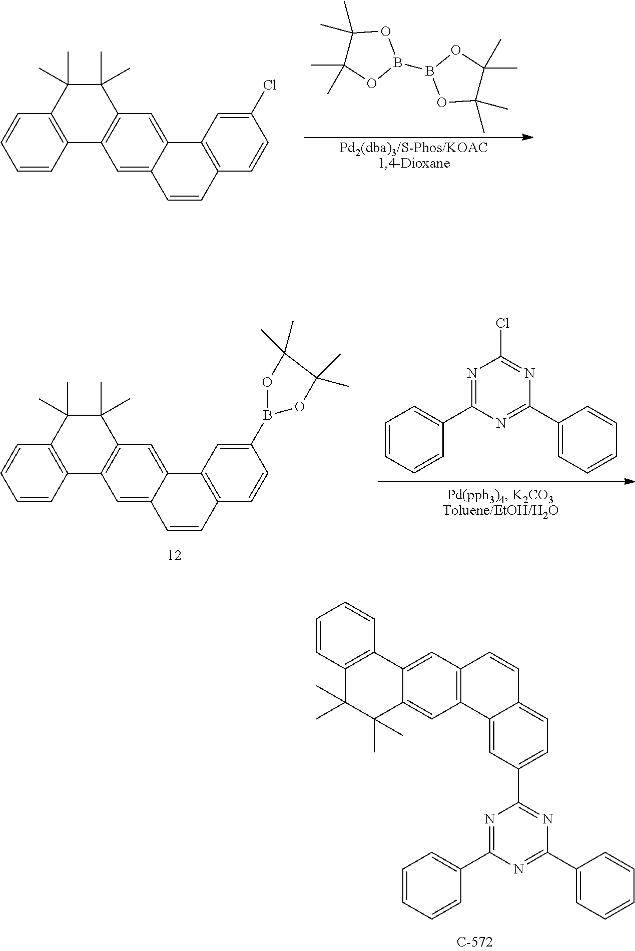

1) Synthesis of Compound 12

9-chloro-5,5,6,6-tetramethyl-5,6-dihydrobenzo[k]tetraphene (7.5 g, 20.2 mmol), 4,4,4′,4′,5,5,5′,5′-octamethyl-2,2′-bi(1,3,2-dioxaborolane) (10.3 g, 40.4 mmol), Pd₂(dba)₃ (0.92 g, 1.01 mmol), S-Phos (0.83 g, 2.02 mmol), KOAC (4.95 g, 50.5 mmol), and 100 mL of 1,4-dioxane were added into a flask, and then stirred at 180° C. After completion of the reaction, only the organic layer was separated by adding MC and H₂O, and then the solvent was removed by filtration under reduced pressure. Thereafter, it was separated by column chromatography, and then MeOH was added thereto. Next, the resulting solid was filtered under reduced pressure to obtain compound 12 (9.6 g, yield: 95%).

2) Synthesis of Compound C-572

Compound 12 (9.6 g, 20.7 mmol), 2-chloro-4,6-diphenyl-1,3,5-triazine (5.3 g, 19.7 mmol), Pd(pph₃)₄ (1.13 g, 0.98 mmol), K₂CO₃ (5.4 g, 39.4 mmol), 200 mL of toluene, 40 mL of EtOH, and 40 mL of H₂O were added into a flask, and then stirred at 160° C. After completion of the reaction, only the organic layer was separated by adding EA and H₂O, and then the solvent was removed by filtration under reduced pressure. Thereafter, it was separated by column chromatography, and then MeOH was added thereto. Next, the resulting solid was filtered under reduced pressure compound C-572 (8.5 g, yield: 80%).

| | MW | color | M.P |
|---|---|---|---|
| C-572 | 567.44 | white | 305° C. |

Hereinafter, the luminous property of the organic electroluminescent device comprising the organic electroluminescent compound according to the present disclosure will be explained in order to understand the present disclosure in detail.

### [Device Example 1-1] Preparation of an OLED Comprising the Organic Electroluminescent Compound According to the Present Disclosure

An OLED was produced by using the organic electroluminescent compound of the present disclosure. First, a transparent electrode indium tin oxide (ITO) thin film (10 0/sq) on a glass substrate for an OLED (GEOMATEC CO., LTD., Japan) was subject to an ultrasonic washing with acetone, ethanol, and distilled water, sequentially, and thereafter was stored in isopropanol and then used. After evacuating until the degree of vacuum in the chamber reached 10⁻⁶ torr, the ITO substrate was mounted on a substrate holder of a vacuum vapor deposition apparatus. Then, compound HT-1 was introduced into a cell of the vacuum vapor deposition apparatus, and compound HI-1 was introduced into another cell of the vacuum vapor deposition apparatus. The two materials were evaporated at different rates and were deposited in a doping amount of 3 wt %, respectively, to form a hole injection layer having a thickness of 10 nm on the ITO substrate. Next, compound HT-1 was introduced into a cell of the vacuum vapor deposition apparatus and was evaporated by applying an electric current to the cell, thereby forming a first hole transport layer having a thickness of 90 nm on the hole injection layer. Next, compound C1-14 described in the following Table 1 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: Compound RH was introduced into a cell of the vacuum vapor deposition apparatus as a host, and compound D-39 was introduced into another cell as a dopant. The two materials were evaporated and the dopant was deposited in a doping amount of 2 wt % based on the total amount of the host and dopant to form a light-emitting layer having a thickness of 40 nm on the second hole transport layer. Next, compound ET and compound EI in another two cells were evaporated at a rate of 1:1 to deposit an electron transport layer having a thickness of 35 nm on the light-emitting layer. Next, after depositing compound EI as an electron injection layer having a thickness of 2 nm, an AI cathode having a thickness of 80 nm was deposited on the electron injection layer by another vacuum vapor deposition apparatus. Thus, an OLED was produced.

### [Comparative Example 1-1] Preparation of an OLED Comprising the Conventional Compound

An OLED was produced in the same manner as in Device Example 1-1, except that compound NPB was used as a material of the second hole transport layer.

The driving voltage, the luminous efficiency, and the color coordinates at a luminance of 1,000 nits, and the time taken for luminance to decrease from 100% to 95% at a luminance of 10,000 nits (lifespan; T95) of the OLEDs according to Device Example 1-1 and Comparative Example 1-1 produced as described above, are measured and the results thereof are shown in Table 1-1 below:

TABLE 1-1

| | Material for | | | | | |
|---|---|---|---|---|---|---|
| | Second Hole | Driving | Luminous | CIE | | |
| | Transport | Voltage | Efficiency | Color Coordinates | | Lifespan |
| | Layer | (V) | (cd/A) | x | y | (T95, hr) |
| Device | C1-14 | 2.9 | 29.7 | 0.662 | 0.338 | 122 |
| Example | | | | | | |
| 1-1 | | | | | | |
| Comparative | NPB | 4.0 | 24.7 | 0.661 | 0.338 | 77.8 |
| Example | | | | | | |
| 1-1 | | | | | | |

### [Device Examples 1-2 to 14] Preparation of OLEDs Comprising the Organic Electroluminescent Compound According to the Present Disclosure

OLEDs were produced in the same manner as in Device Example 1-1, except that compound RH-2 was used as a host of the light-emitting layer and the compound described in the following Table 1-2 was used as material of the second hole transport layer.

### [Comparative Example 1-2] Preparation of an OILED Comprising the Conventional Compound

An OLED was produced in the same manner as in Device Example 1-1, except that compound RH-2 was used as a host of the light-emitting layer and the compound described in the following Table 1-2 was used as material of the second hole transport layer.

The time taken for luminance to decrease from 100% to 95% at a luminance of 10,000 nits (lifespan; T95) of the OLEDs according to Device Examples 1-2 to 1-4 and Comparative Example 1-2 produced as described above, are measured and the results thereof are shown in Table 1-2 below:

TABLE 1-2

| | Material for Seconf Hole | Lifespan |
|---|---|---|
| | Transport Layer | (T95, hr) |
| Device Example 1-2 | C2-31 | 141 |
| Device Example 1-3 | C2-8 | 134 |
| Device Example 1-4 | C-317 | 273 |
| Comparative Example 1-2 | A | 68 |

By comprising the organic electroluminescent compound according to the present disclosure in the hole transport zone, an organic electroluminescent device having low driving voltage, high luminous efficiency, and long lifespan properties can be provided.

### [Device Examples 2-1 and 2-2] Preparation of OLEDs Comprising the Organic Electroluminescent Compound According to the Present Disclosure

OLEDs according to the present disclosure were produced. Firstly, a transparent electrode indium tin oxide (ITO) thin film (10 n/sq) on a glass substrate for an OLED (GEOMATEC CO.. LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 was introduced into a cell of the vacuum vapor deposition apparatus, and compound HT-1 was introduced into another cell of the vacuum vapor deposition apparatus. The two materials were evaporated at different rates and compound HI-1 was deposited in a doping amount of 3 wt % based on the total amount of the two materials to form a hole injection layer having a thickness of 10 nm. Next, compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: The compound shown in Table 2 below was introduced into a cell of the vacuum vapor deposition apparatus as a host, and compound D-50 was introduced into another cell as a dopant. Simultaneously, the dopant material was evaporated at different rate, and was deposited in a doping amount of 10 wt % based on the total amount of the host and dopant to form a light-emitting layer having a thickness of 40 nm on the hole transport layer. Next, compound ET and compound EI as a material for the electron transport layer were deposited at a weight ratio of 40:60 to form an electron transport layer having a thickness of 35 nm on the light-emitting layer. After depositing compound EI as a material for an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials was purified by vacuum sublimation under 10⁻⁶ torr.

### [Device Examples 2-3 and 2-4] Preparation of OLEDs Comprising the Plurality of Host Materials According to the Present Disclosure

OLEDs were produced in the same manner as in Device Example 2-1, except that the compounds shown in Table 2 below were used as host materials and the two host materials were evaporated at a different rate of 1:2 to deposit a light-emitting layer.

### [Comparative Example 2] Preparation of an OLED Comprising the Conventional Compound as a Host

An OLED was produced in the same manner as in Device Example 2-1, except that only compound CBP was used as a host material to deposit a light-emitting layer, and compound BAlq was used as a material for a hole blocking layer to deposit a hole blocking layer having a thickness of 5 nm on the light-emitting layer, and then, compounds ET and EI as materials for an electron transport layer were deposited at a weight ratio of 40:60 to form an electron transport layer having a thickness of 30 nm on the hole blocking layer.

The driving voltage, the luminous efficiency, the power efficiency, and the light-emitting color at a luminance of 1,000 nits, and the time taken for luminance to decrease from 100% to 95% at a luminance of 20,000 nits (lifespan; T95) of the OLEDs according to Device Examples 2-1 to 2-4 and Comparative Example 2 produced as described above, are measured and the results thereof are shown in Table 2 below:

TABLE 2

| | Host Material | | | | | | |
|---|---|---|---|---|---|---|---|
| | First | Second | Driving | Luminous | Power | | |
| | Host | Host | Voltage | Efficiency | Efficiency | Light-emitting | Lifespan |
| | Material | Material | (V) | (cd/A) | (Im/W) | Color | (T95, hr) |
| Device | C-14 | | 2.9 | 91.2 | 99.1 | Green | 4.3 |
| Example | | | | | | | |
| 2-1 | | | | | | | |
| Device | C-77 | | 2.9 | 69.6 | 74.9 | Green | 18.2 |
| Example | | | | | | | |
| 2-2 | | | | | | | |
| Device | C-14 | H-2 | 3.2 | 91.8 | 89.4 | Green | 56.9 |
| Example | | | | | | | |
| 2-3 | | | | | | | |
| Device | C-470 | H-2 | 3.3 | 92.5 | 89.4 | Green | 59.5 |
| Example | | | | | | | |
| 2-4 | | | | | | | |

| | | | | | | |
|---|---|---|---|---|---|---|
| Comparative | CBP | 5.7 | 75.8 | 42.0 | Green | 0.2 |
| Example | | | | | | |
| 2 | | | | | | |

By comprising the organic electroluminescent compound according to the present disclosure and a plurality of host materials comprising the same in the light-emitting layer, a long lifespan organic electroluminescent device having not only low driving voltage and excellent luminous characteristics, but also significantly improved lifespan, can be provided, compared to the OLED comprising conventional host materials.

### [Device Example 3-1] Preparation of an OLED Comprising the Compound According to the Present Disclosure

An OLED was produced by using the organic electroluminescent compound of the present disclosure. First, a transparent electrode indium tin oxide (ITO) thin film (10 Ω/sq) on a glass substrate for an OLED (GEOMATEC CO., LTD., Japan) was subjected to an ultrasonic washing with acetone, ethanol, and isopropyl alcohol, sequentially, and thereafter was stored in isopropanol and then used. After evacuating until the degree of vacuum in the chamber reached 10⁻⁶ torr, the ITO substrate was mounted on a substrate holder of a vacuum vapor deposition apparatus. Then, compound HT-1 was introduced into a cell of the vacuum vapor deposition apparatus, and compound HI-1 was introduced into another cell of the vacuum vapor deposition apparatus. The two materials were evaporated at different rates and the respective compounds were deposited in a doping amount of 3 wt % to form a hole injection layer having a thickness of 10 nm on the ITO substrate. Next, compound HT-1 was then introduced into a cell of the vacuum vapor deposition apparatus and was evaporated by applying an electric current to the cell, thereby forming a first hole transport layer having a thickness of 75 nm on the hole injection layer. Next, compound HT-3 was then introduced into another cell of the vacuum vapor deposition apparatus and was evaporated by applying an electric current to the cell, thereby forming a second hole transport layer having a thickness of 5 nm on the first transport layer. After forming the hole injection layer and the hole transport layers, a light-emitting layer was formed thereon as follows: The compound BH-1 was introduced into a cell of the vacuum vapor deposition apparatus as a host, and compound BD was introduced into another cell as a dopant. Thereafter, the two materials were evaporated, and the dopant was deposited in a doping amount of 2 wt % based on the total amount of the host and dopant to form a light-emitting layer having a thickness of 20 nm on the second hole transport layer. Next, compound C-14 was deposited as a hole blocking material to form a hole blocking layer having a thickness of 5 nm. Compound ET and compound EI in another two cells were evaporated at a rate of 1:1 to deposit an electron transport layer having a thickness of 30 nm on the hole blocking layer. After depositing compound EI as an electron injection layer having a thickness of 2 nm, an Al cathode having a thickness of 80 nm was deposited by another vacuum vapor deposition apparatus. Thus, an OLED was produced.

### [Comparative Example 3-1] Preparation of an OLED Comprising a Conventional Compound

An OLED was produced in the same manner as in Device Example 3-1, except that compound ET and compound EI as electron transport layers were evaporated at a rate of 1:1 to deposit an electron transport layer having a thickness of 33 nm on the light-emitting layer without depositing a hole blocking layer.

The driving voltage, the current efficiency, and the CIE color coordinates at a luminance of 1,000 nits, of the OLEDs according to Device Example 3-1 and Comparative Example 3-1 produced as described above, are measured and the results thereof are shown in Table 3-1 below:

TABLE 3-1

| | Material | | | | |
|---|---|---|---|---|---|
| | for Hole | Driving | Current | CIE color | |
| | Blocking | Voltage | Efficiency | coordinates | |
| | Layer | (V) | (cd/A) | x | y |
| Device | C-14 | 4.0 | 4.4 | 0.141 | 0.055 |
| Example | | | | | |
| 3-1 | | | | | |
| Comparative | — | 4.2 | 3.7 | 0.141 | 0.054 |
| Example | | | | | |
| 3-1 | | | | | |

### [Device Examples 3-2 and 3-3] Preparation of OLEDs Comprising the Compound According to the Present Disclosure

OLEDs were produced in the same manner as in Device Example 3-1, except that compound BD-1 was used as the dopant material and the compound shown in the Table 3-2 below was used as a material of a hole blocking layer.

### [Comparative Example 3-2] Preparation of an OLED Comprising a Conventional Compound

An OLED was produced in the same manner as in Device Example 3-1, except that compound BD-1 was used as the dopant material, and compounds ET and EI were evaporated at a rate of 1:1 to deposit an electron transport layer having a thickness of 35 nm on the light-emitting layer, without depositing a hole blocking layer.

The driving voltage, the current efficiency, and the CIE color coordinates at a luminance of 1,000 nits, of the OLEDs according to Device Examples 3-2 and 3-3 and Comparative Example 3-2 produced as described above, are measured and the results thereof are shown in Table 3-2 below:

TABLE 3-2

| | Material | | | | |
|---|---|---|---|---|---|
| | for Hole | Driving | Current | CIE color | |
| | Blocking | Voltage | Efficiency | coordinates | |
| | Layer | (V) | (cd/A) | x | y |
| Device | C-14 | 3.9 | 6.1 | 0.131 | 0.084 |
| Example | | | | | |
| 3-2 | | | | | |
| Device | C-76 | 4.0 | 5.7 | 0.131 | 0.082 |
| Example | | | | | |
| 3-3 | | | | | |
| Comparative | — | 4.1 | 5.3 | 0.130 | 0.086 |
| Example | | | | | |
| 3-2 | | | | | |

By comprising the organic electroluminescent compound according to the present disclosure in the hole blocking layer, an organic electroluminescent device having low driving voltage and high luminous efficiency characteristics can be provided.

### [Device Example 4] Preparation of a Red Light-Emitting OLED According to the Present Disclosure

An OLED according to the present disclosure was produced as follows. Firstly, a transparent electrode indium tin oxide (ITO) thin film (10 0/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 was introduced into a cell of the vacuum vapor deposition apparatus, and compound HT-1 was introduced into another cell of the vacuum vapor deposition apparatus. The two materials were evaporated at different rates and compound HI-1 was deposited in a doping amount of 3 wt % based on the total amount of the two materials to form a hole injection layer having a thickness of 10 nm. Next, compound HT-1 was deposited as a first hole transport layer having a thickness of 80 nm on the hole injection layer. Compound HT-4 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The respective first and second host compounds shown in Table 4 below were introduced into the two cells of the vacuum vapor deposition apparatus as hosts, and compound D-39 was introduced into another cell as a dopant. The two host materials were evaporated at a rate of 1:1, and the dopant was evaporated at a different rate, simultaneously. The dopaont was deposited in a doping amount of 3 wt % based on the total amount of the host and dopant to form a light-emitting layer having a thickness of 40 nm on the second hole transport layer. Next, compound ET and compound EI as materials for an electron transport layer were deposited at a weight ratio of 50:50 to form an electron transport layer having a thickness of 35 nm on the light-emitting layer. After depositing compound EI as an electron injection layer having a thickness of 2 nm on the electron transport layer, an Al cathode having a thickness of 80 nm was deposited on the electron injection layer by another vacuum vapor deposition apparatus. Thus, an OLED was produced. Each compound used for all the materials was purified by vacuum sublimation under 10⁻⁶ torr.

### [Comparative Example 4] Preparation of an OLED Comprising a Conventional Compound as a Host

An OLED was produced in the same manner as in Device Example 4, except that compound CBP alone was used as the host of the light-emitting layer.

The driving voltage, the luminous efficiency, and the light-emitting color at a luminance of 1,000 nits, and the time taken for luminance to decrease from 100% to 95% at a luminance of 5,000 nits (lifespan; T95) of the OLEDs according to Device Example 4 and Comparative Example 4 produced as described above, are measured and the results thereof are shown in Table 4 below:

TABLE 4

| | First | Second | Driving | Luminous | Light- | |
|---|---|---|---|---|---|---|
| | Host | Host | Voltage | Efficiency | emitting | Lifespan |
| | Material | Material | (V) | (cd/A) | Color | (T95, hr) |
| Device | C-14 | H-85 | 3.2 | 32.2 | Red | 164 |
| Example 4 | | | | | | |
| Comparative | CBP | — | 9.0 | 14.3 | Red | 0.3 |
| Example 4 | | | | | | |

The compounds used in Device Examples and Comparative Examples above are shown in the following Table 5:

TABLE 5

| | |
|---|---|
| Hole Injection Layer/ Hole Transport Layer | 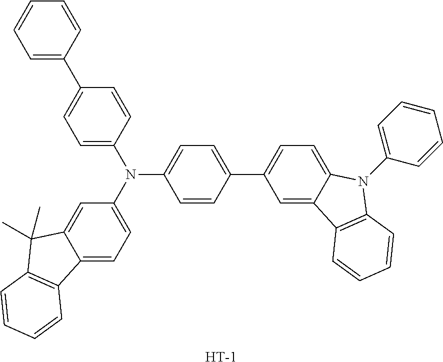 |
| | 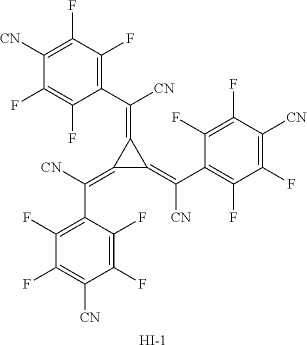 |
| | 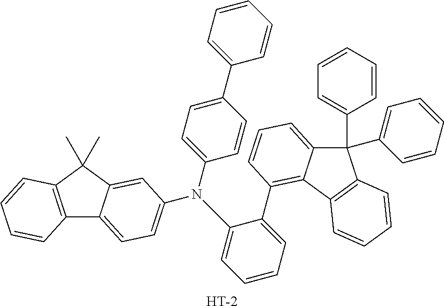 |
| | 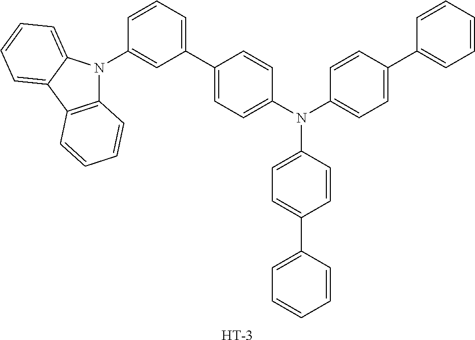 |
| | 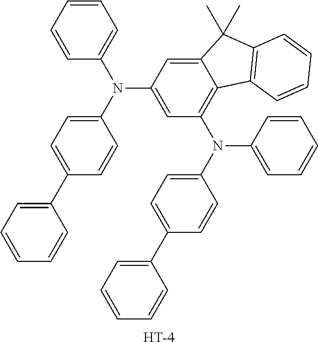 |
| | 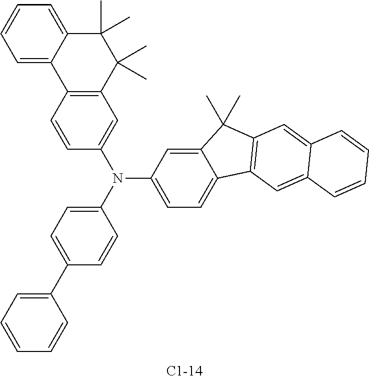 [SMILES: CC1(C)c2cc(N(c3ccc(-c4ccccc4)cc3)c3ccc4c(c3)C(C)(C)C(C)(C)c3ccccc3-4)ccc2-c2cc3ccccc3cc21 ; "C1-14"] |
| | 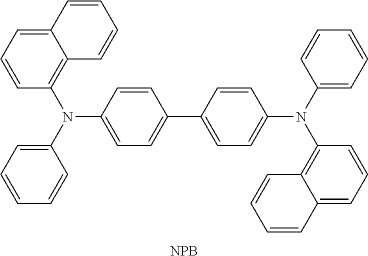 [SMILES: c1ccc(N(c2ccc(-c3ccc(N(c4ccccc4)c4cccc5ccccc45)cc3)cc2)c2cccc3ccccc23)cc1 ; "NPB"] |
| | 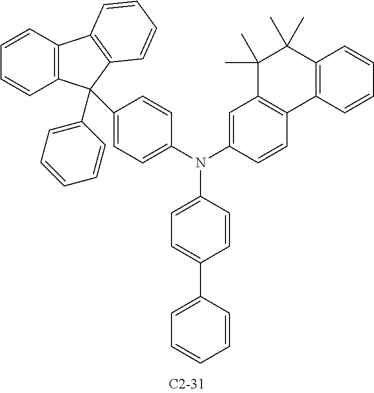 [SMILES: CC1(C)c2ccccc2-c2ccc(N(c3ccc(-c4ccccc4)cc3)c3ccc(C4(c5ccccc5)c5ccccc5-c5ccccc54)cc3)cc2C1(C)C] |
| | 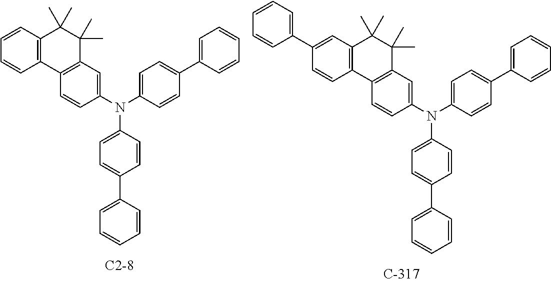 |
| | 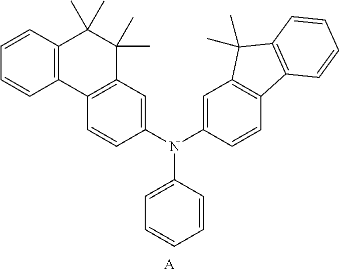 |
| Light- Emitting Layer | 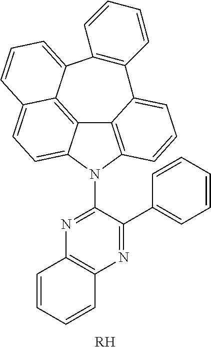 |
| | 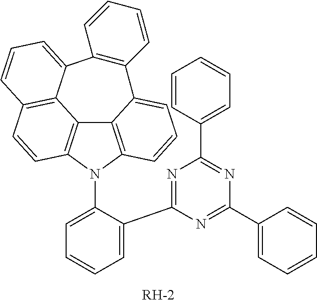 [SMILES: c1ccc(-c2nc(-c3ccccc3)nc(-c3ccccc3-n3c4cccc5c4c4c6c(cccc6ccc43)-c3ccccc3-5)n2)cc1] |
| | 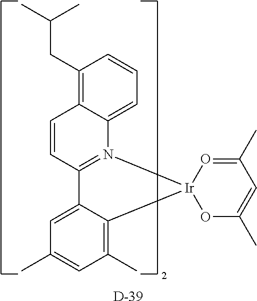 |
| | 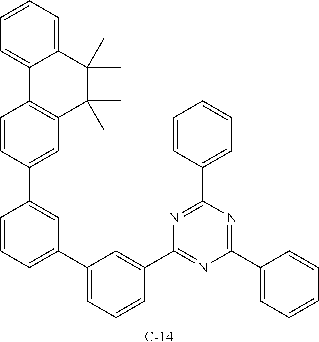 |
| | 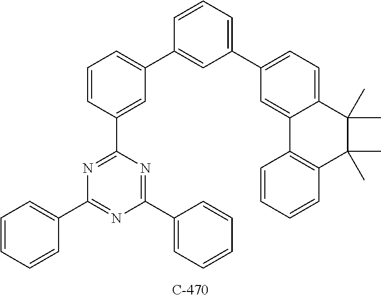 [SMILES: CC1(C)c2ccccc2-c2cc(-c3cccc(-c4cccc(-c5nc(-c6ccccc6)nc(-c6ccccc6)n5)c4)c3)ccc2C1(C)C] |
| | 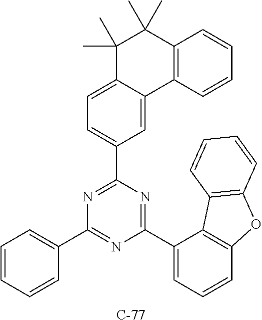 |
| | 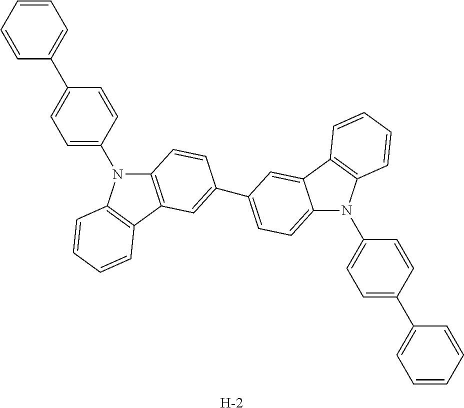 |
| | 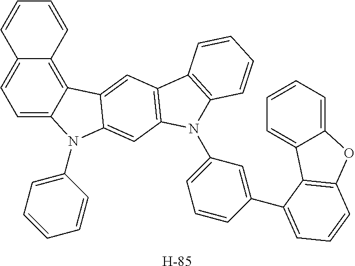 |
| | 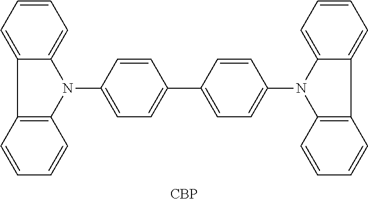 [SMILES: c1ccc2c(c1)c1ccccc1n2-c1ccc(-c2ccc(-n3c4ccccc4c4ccccc43)cc2)cc1 ; "CBP"] |
| | 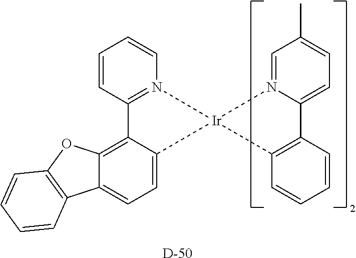 |
| | 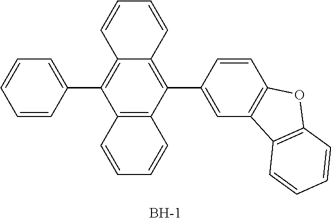 |
| | 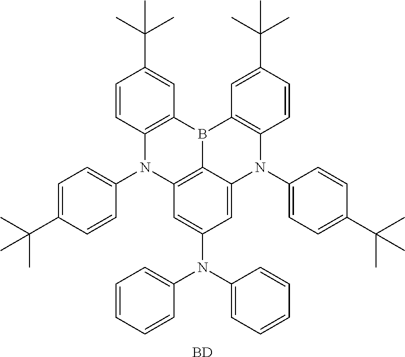 |
| | 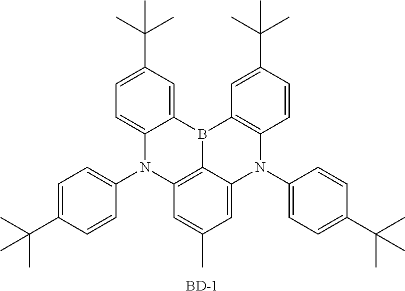 |
| Hole Blocking Layer/ Electron Transport Layer/ Electron Injection Layer | 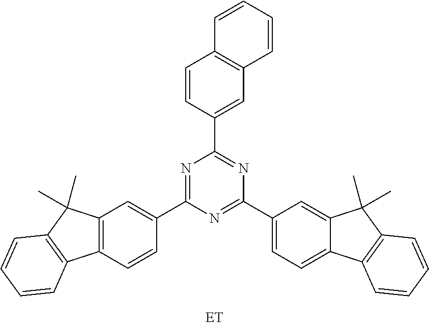 [SMILES: CC1(C)c2ccccc2-c2ccc(-c3nc(-c4ccc5c(c4)C(C)(C)c4ccccc4-5)nc(-c4ccc5ccccc5c4)n3)cc21] . |
| | 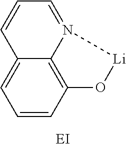 |
| | 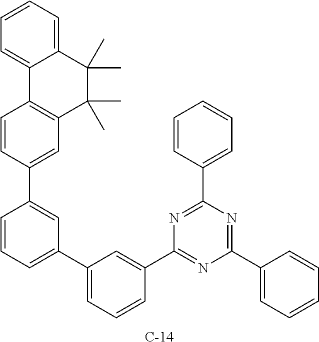 |
| | 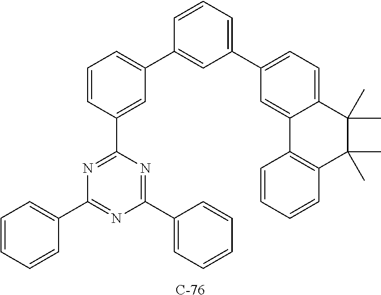 |
| | 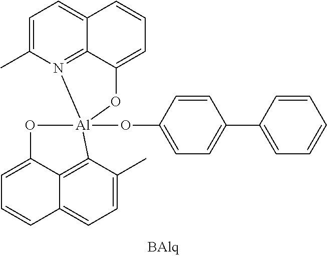 |

Further, in the organic electroluminescent compound represented by formula 1 according to the present disclosure, the LUMO (lowest unoccupied molecular orbital) energy level, HOMO (highest unoccupied molecular orbital) energy level, and triplet energy of the compounds where R₅ to R₁₂ are linked to the adjacent substituent(s) to form a benzene ring or or a naphthalene ring, are measured, respectively, and the results thereof are shown in Table 6 below:

TABLE 6

| | 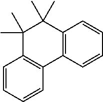 [SMILES: CC1(C)c2ccccc2-c2ccccc2C1(C)C] | 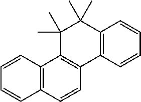 [SMILES: CC1(C)c2ccccc2-c2ccc3ccccc3c2C1(C)C] | 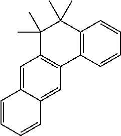 [SMILES: CC1(C)c2ccccc2-c2cc3ccccc3cc2C1(C)C] | 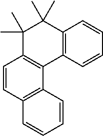 [SMILES: CC1(C)c2ccccc2-c2c(ccc3ccccc23)C1(C)C] |
|---|---|---|---|---|
| LUMO (ev) | −0.741 | −1.138 | −1.153 | −1.184 |
| HOMO (ev) | −5.684 | −5.426 | 5.526 | −5.338 |
| Triplet (ev) | 3.008 | 2.475 | 2.549 | 2.399 |

*The structure was optimized by applying the hybrid density functional theory (hybrid DFT) (B3LYP) and 6-31G(d) basis sets using Gaussian's quantum chemistry calculation program Gaussian16, and the triplet state was calculated by using TD-DFT (time dependent DFT).

Referring to Table 6 above, in the organic electroluminescent compound represented by formula 1 according to the present disclosure, even when R₅ to R₆ and/or R₉ to R₁₂ are linked to the adjacent substituent(s) to form a benzene ring or or a naphthalene ring, it can be confirmed that it has the energy level of the main core usable as a material for an OLED according to the present disclosure.

1. An organic electroluminescent compound represented by the following formula 1: 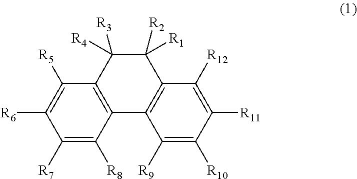
wherein
R₁ to R₄ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent(s) to form a ring(s):
R₅ to R₁₂ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of an (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s);
provided that at least one of R₁ to R₁₂ represent(s) *-(L₁)ₐ-(Ar₁)_{b};
L₁ represents a single bond, a substituted or unsubstituted (C6-C30)arylene, or a substituted or unsubstituted (3- to 30-membered)heteroarylene;
Ar₁ represents a substituted or unsubstituted (C6-C30)aryl, a substituted or unsubstituted (3- to 30-membered)heteroaryl, or —N—(Ar₂)(Ar₃);
Ar₂ and Ar₃ each independently represent a substituted or unsubstituted (C1-C30)alkyl, a substituted or unsubstituted (C2-C30)alkenyl, a substituted or unsubstituted fused ring of an (C3-C30) aliphatic ring and a (C6-C30) aromatic ring, a substituted or unsubstituted (C6-C30)aryl, or a substituted or unsubstituted (3- to 30-membered)heteroaryl; and
a represents an integer of 1 or 2, b represents an integer of 1 to 4, and when a and b are 2 or more, each of L₁ and each of Ar₁ may be the same or different;
provided that the compounds where R₅ to R₁₀ and R₁₂ represent hydrogen and R₁₁ include a substituted amino group are excluded.

2. The organic electroluminescent compound according to claim 1, wherein
Ar₁ represents a substituted or unsubstituted (3- to 30-membered)heteroaryl containing at least one N. or —N—(Ar₂)(Ar₃); and
L₁ represents a single bond or a substituted or unsubstituted (C6-C30)arylene.

3. The organic electroluminescent compound according to claim 1, wherein the compound represented by formula 1 is represented by any one of the following formulas 1-1 to 1-4: 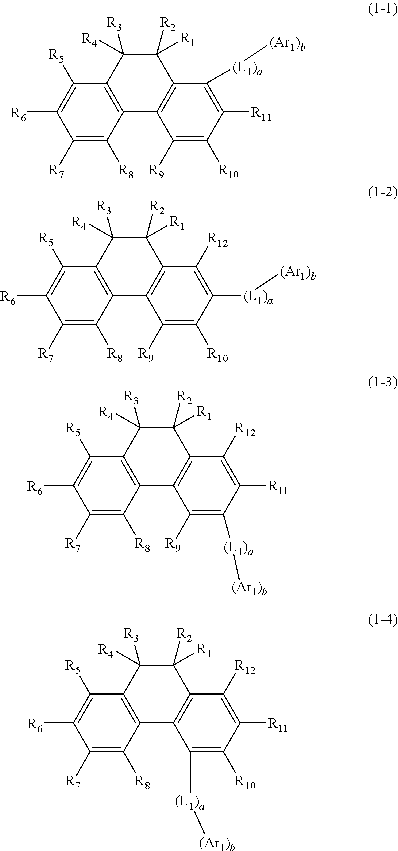
wherein
R₁ to R₁₂, L₁, Ar₁, a, and b are as defined in claim 1.

4. The organic electroluminescent compound according to claim 1, wherein the compound represented by formula 1 is represented by any one of the following formulas 1-5 to 1-13: 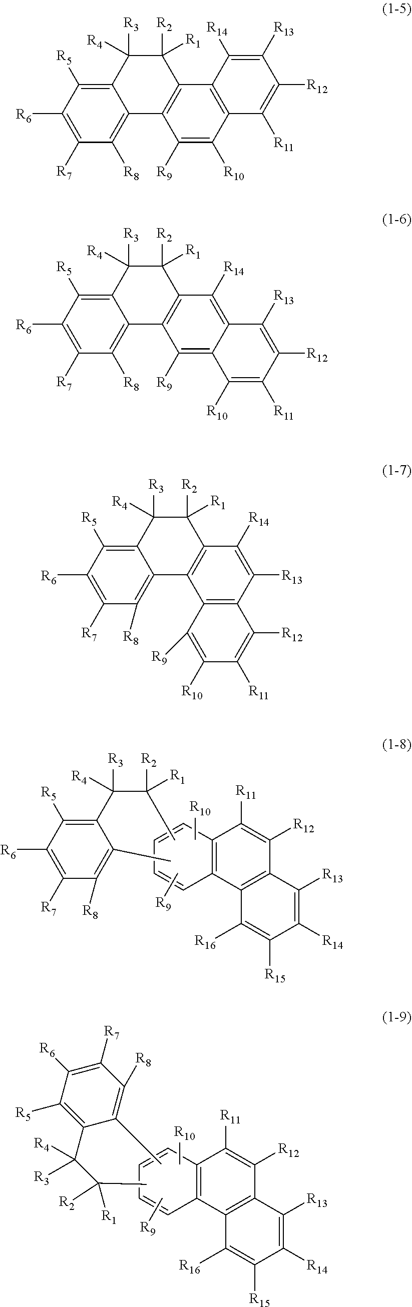 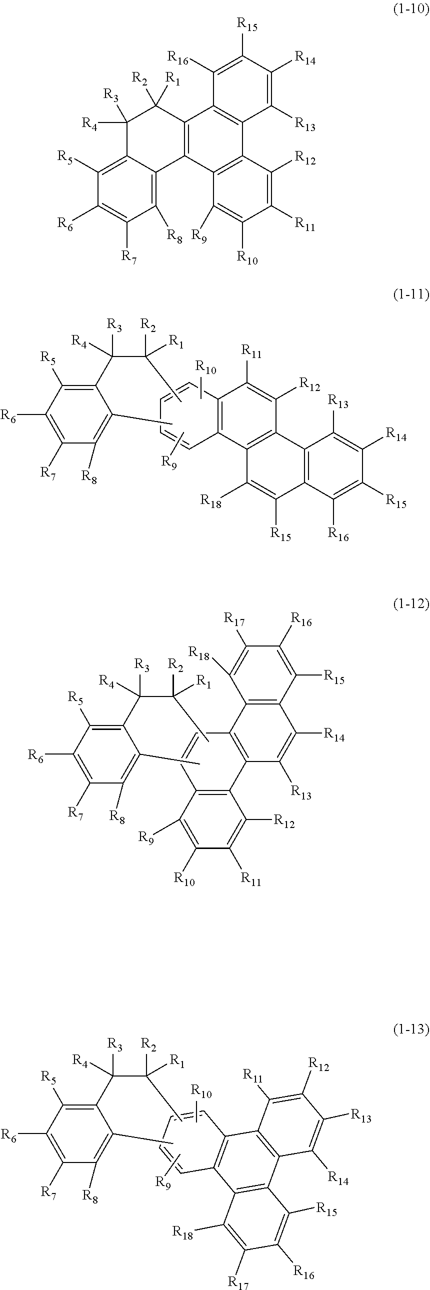
wherein
R₁ to R₁₂ are as defined in claim 1;
R₁₃ to R₁₈ each independently represent *-(L₁)ₐ-(Ar₁)_{b}, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of an (C3-C30) aliphatic ring and a (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s);
provided that at least one of R₁ to R₁₄ in the formulas 1-5 to 1-7, at least one of R₁ to R₁₆ in the formulas 1-8 to 1-10, and at least one of R₁ to R₁₈ in the formulas 1-11 to 1-13 represent(s) *-(L₁)ₐ-(Ar₁)_{b}; and
L₁, Ar₁, a, and b are as defined in claim 1.

5. The organic electroluminescent compound according to claim 1, wherein L₁ represents a single bond, a substituted or unsubstituted phenylene, a substituted or unsubstituted biphenylene, a substituted or unsubstituted terphenylene, a substituted or unsubstituted naphthylene, a substituted or unsubstituted phenanthrenylene, a substituted or unsubstituted triphenylenylene, a substituted or unsubstituted fluorenylene, a substituted or unsubstituted pyridylene, a substituted or unsubstituted triazinylene, a substituted or unsubstituted carbazolylene, a substituted or unsubstituted quinoxalinylene, a substituted or unsubstituted quinazolinylene, a substituted or unsubstituted dibenzofuranylene, or a substituted or unsubstituted benzoquinoxalinylene.

6. The organic electroluminescent compound according to claim 1, wherein the substituted or unsubstituted (3- to 30-membered)heteroaryl of Ar₁ represents a substituted or unsubstituted pyridyl, a substituted or unsubstituted pyrimidinyl, a substituted or unsubstituted triazinyl, a substituted or unsubstituted pyrazinyl, a substituted or unsubstituted quinolinyl, a substituted or unsubstituted quinazolinyl, a substituted or unsubstituted quinoxalinyl, a substituted or unsubstituted benzoquinolinyl, a substituted or unsubstituted benzoquinazolinyl, a substituted or unsubstituted benzoquinoxalinyl, a substituted or unsubstituted dibenzoquinolinyl, a substituted or unsubstituted dibenzoquinazolinyl, a substituted or unsubstituted dibenzoquinoxalinyl, a substituted or unsubstituted indenopyridyl, a substituted or unsubstituted indenopyrimidinyl, a substituted or unsubstituted indenopyrazinyl, a substituted or unsubstituted benzofuropyridyl, a substituted or unsubstituted benzofuropyrmidinyl, a substituted or unsubstituted benzofuropyrazinyl, a substituted or unsubstituted benzothiopyridyl, a substituted or unsubstituted benzothiopyrimidinyl, a substituted or unsubstituted benzothiopyrazinyl, a substituted or unsubstituted carbazolyl, a substituted or unsubstituted benzocarbazolyl, a substituted or unsubstituted dibenzofuranyl, or a substituted or unsubstituted dibenzothiophenyl.

7. The organic electroluminescent compound according to claim 1, wherein the substituted or unsubstituted (C6-C30)aryl of Ar₁ represents a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted triphenylenyl, or a substituted or unsubstituted phenanthrenyl.

8. The organic electroluminescent compound according to claim 1, wherein Ar₂ and Ar₃ each independently represent a substituted or unsubstituted phenyl, a substituted or unsubstituted naphthyl, a substituted or unsubstituted biphenyl, a substituted or unsubstituted fluorenyl, a substituted or unsubstituted phenanthrenyl, a substituted or unsubstituted chrysenyl, a substituted or unsubstituted dibenzofuranyl, a substituted or unsubstituted dibenzothiophenyl, a substituted or unsubstituted carbazolyl, a substituted or unsubstituted benzofluorenyl, or a substituted or unsubstituted dihydrophenanthrenyl.

9. The organic electroluminescent compound according to claim 1, wherein the compound represented by formula 1 is selected from the following compounds:

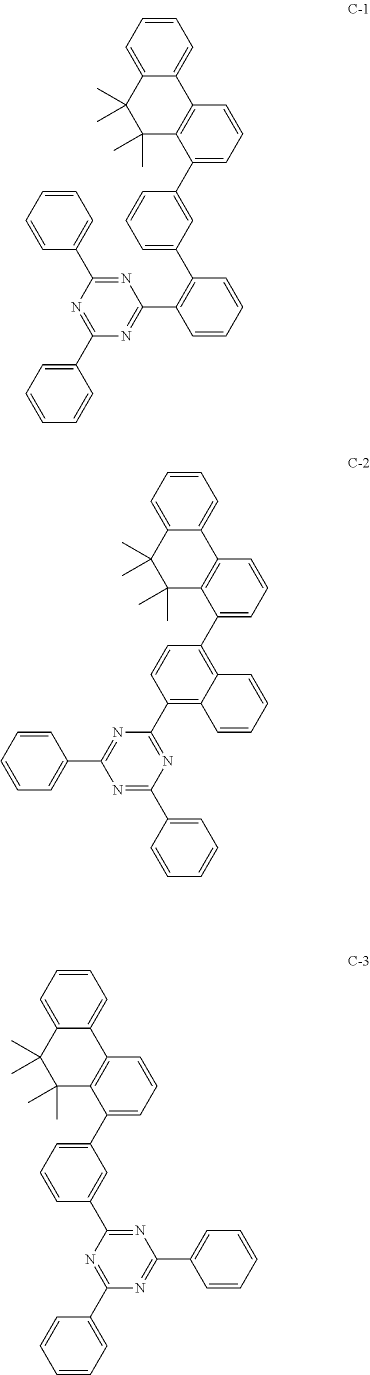 [SMILES: CC1(C)c2ccccc2-c2cccc(-c3cccc(-c4ccccc4-c4nc(-c5ccccc5)nc(-c5ccccc5)n4)c3)c2C1(C)C ; CC1(C)c2ccccc2-c2cccc(-c3ccc(-c4nc(-c5ccccc5)nc(-c5ccccc5)n4)c4ccccc34)c2C1(C)C ; CC1(C)c2ccccc2-c2cccc(-c3cccc(-c4nc(-c5ccccc5)nc(-c5ccccc5)n4)c3)c2C1(C)C]

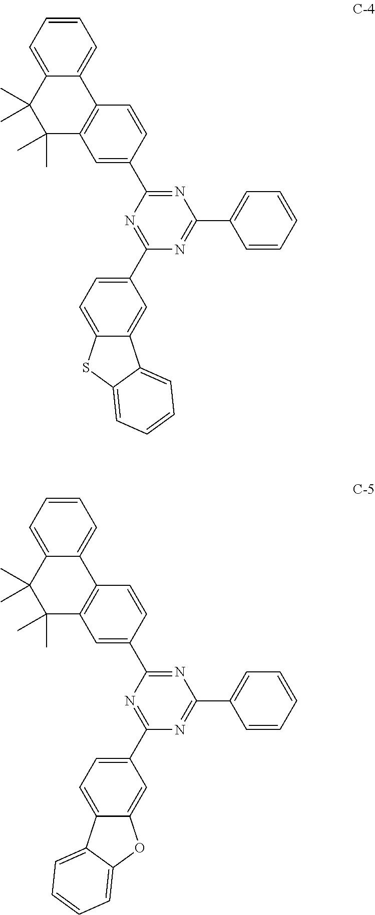 [SMILES: CC1(C)c2ccccc2-c2ccc(-c3nc(-c4ccccc4)nc(-c4ccc5sc6ccccc6c5c4)n3)cc2C1(C)C ; CC1(C)c2ccccc2-c2ccc(-c3nc(-c4ccccc4)nc(-c4ccc5c(c4)oc4ccccc45)n3)cc2C1(C)C]

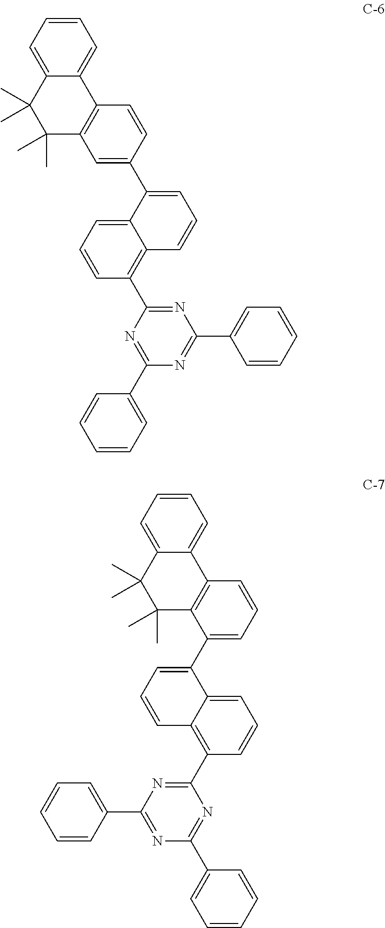 [SMILES: CC1(C)c2ccccc2-c2ccc(-c3cccc4c(-c5nc(-c6ccccc6)nc(-c6ccccc6)n5)cccc34)cc2C1(C)C ; CC1(C)c2ccccc2-c2cccc(-c3cccc4c(-c5nc(-c6ccccc6)nc(-c6ccccc6)n5)cccc34)c2C1(C)C]

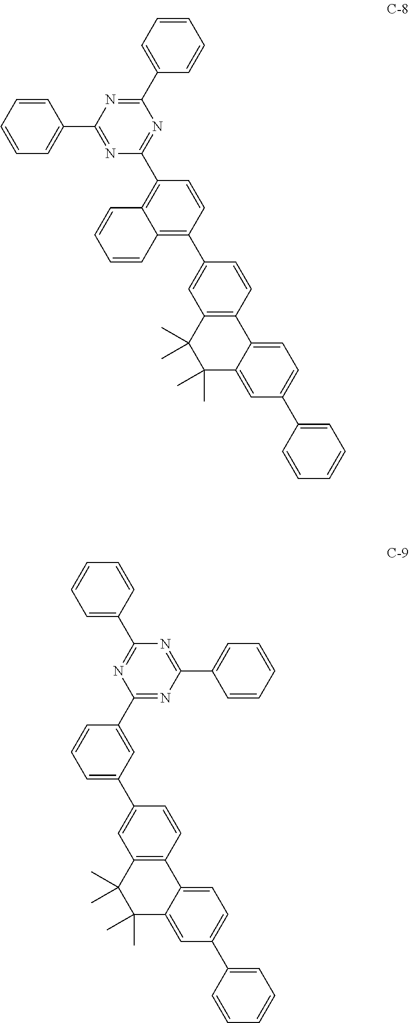 [SMILES: CC1(C)c2cc(-c3ccccc3)ccc2-c2ccc(-c3ccc(-c4nc(-c5ccccc5)nc(-c5ccccc5)n4)c4ccccc34)cc2C1(C)C ; CC1(C)c2cc(-c3ccccc3)ccc2-c2ccc(-c3cccc(-c4nc(-c5ccccc5)nc(-c5ccccc5)n4)c3)cc2C1(C)C]

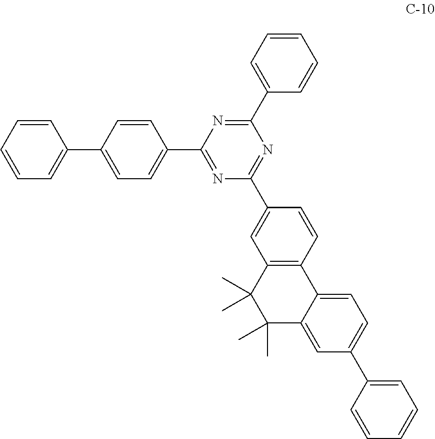 [SMILES: CC1(C)c2cc(-c3ccccc3)ccc2-c2ccc(-c3nc(-c4ccccc4)nc(-c4ccc(-c5ccccc5)cc4)n3)cc2C1(C)C]

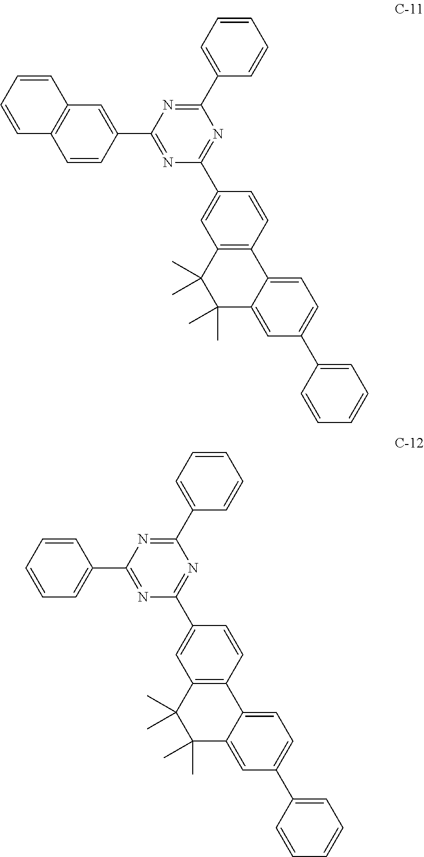 [SMILES: CC1(C)c2cc(-c3ccccc3)ccc2-c2ccc(-c3nc(-c4ccccc4)nc(-c4ccc5ccccc5c4)n3)cc2C1(C)C ; CC1(C)c2cc(-c3ccccc3)ccc2-c2ccc(-c3nc(-c4ccccc4)nc(-c4ccccc4)n3)cc2C1(C)C]

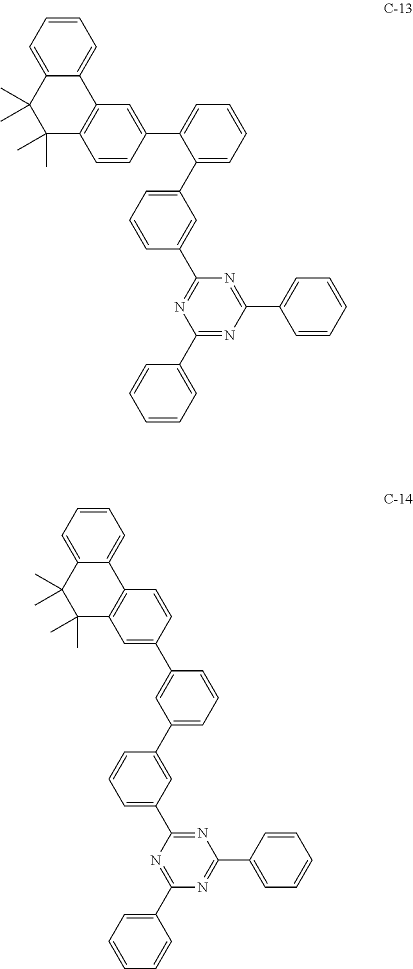 [SMILES: CC1(C)c2ccccc2-c2cc(-c3ccccc3-c3cccc(-c4nc(-c5ccccc5)nc(-c5ccccc5)n4)c3)ccc2C1(C)C ; CC1(C)c2ccccc2-c2ccc(-c3cccc(-c4cccc(-c5nc(-c6ccccc6)nc(-c6ccccc6)n5)c4)c3)cc2C1(C)C]

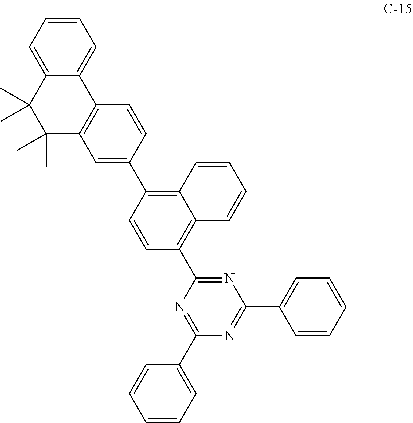 [SMILES: CC1(C)c2ccccc2-c2ccc(-c3ccc(-c4nc(-c5ccccc5)nc(-c5ccccc5)n4)c4ccccc34)cc2C1(C)C]

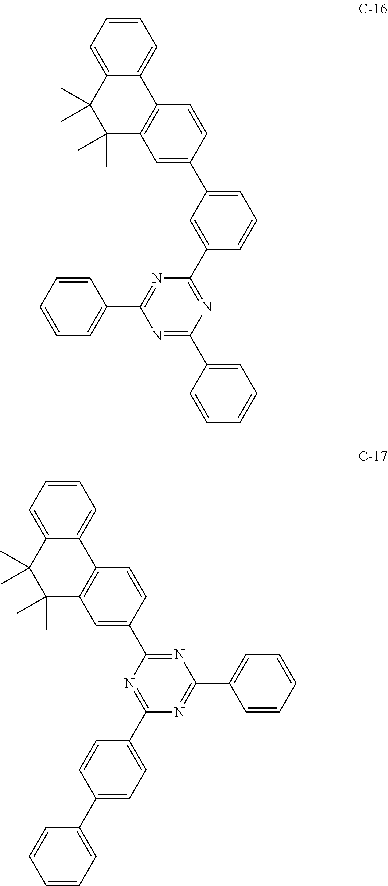 [SMILES: CC1(C)c2ccccc2-c2ccc(-c3cccc(-c4nc(-c5ccccc5)nc(-c5ccccc5)n4)c3)cc2C1(C)C ; CC1(C)c2ccccc2-c2ccc(-c3nc(-c4ccccc4)nc(-c4ccc(-c5ccccc5)cc4)n3)cc2C1(C)C]

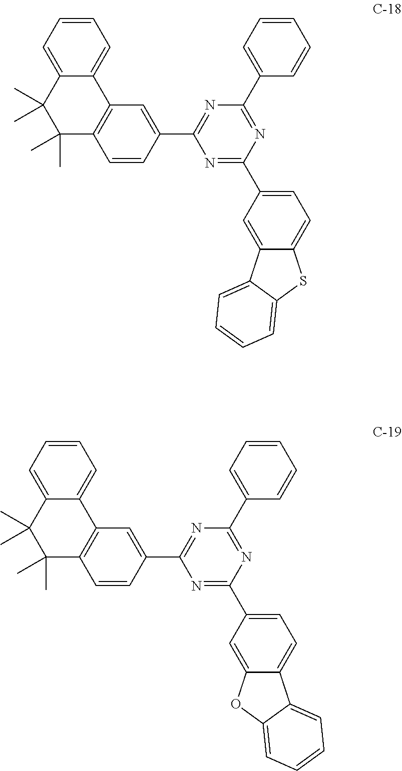 [SMILES: CC1(C)c2ccccc2-c2cc(-c3nc(-c4ccccc4)nc(-c4ccc5sc6ccccc6c5c4)n3)ccc2C1(C)C ; CC1(C)c2ccccc2-c2cc(-c3nc(-c4ccccc4)nc(-c4ccc5c(c4)oc4ccccc45)n3)ccc2C1(C)C]

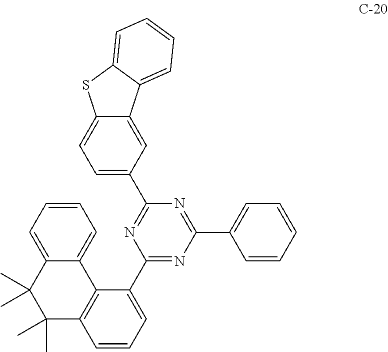 [SMILES: CC1(C)c2ccccc2-c2c(-c3nc(-c4ccccc4)nc(-c4ccc5sc6ccccc6c5c4)n3)cccc2C1(C)C]

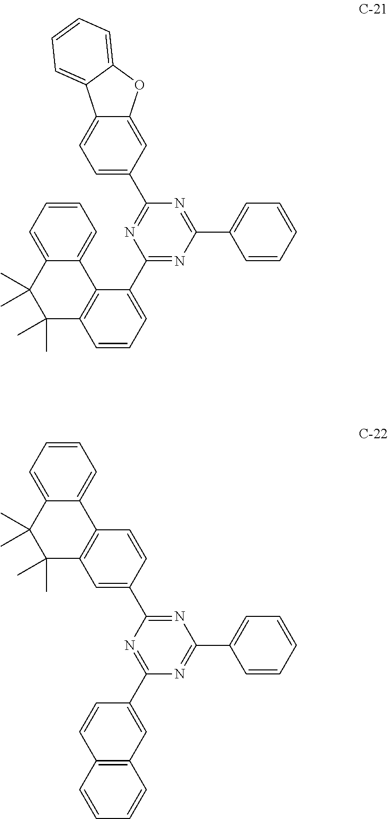 [SMILES: CC1(C)c2ccccc2-c2c(-c3nc(-c4ccccc4)nc(-c4ccc5c(c4)oc4ccccc45)n3)cccc2C1(C)C ; CC1(C)c2ccccc2-c2ccc(-c3nc(-c4ccccc4)nc(-c4ccc5ccccc5c4)n3)cc2C1(C)C]

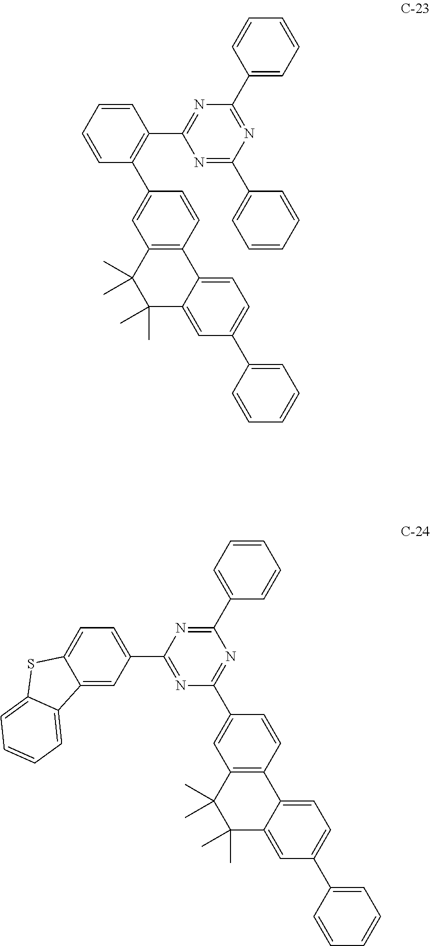 [SMILES: CC1(C)c2cc(-c3ccccc3)ccc2-c2ccc(-c3ccccc3-c3nc(-c4ccccc4)nc(-c4ccccc4)n3)cc2C1(C)C ; CC1(C)c2cc(-c3ccccc3)ccc2-c2ccc(-c3nc(-c4ccccc4)nc(-c4ccc5sc6ccccc6c5c4)n3)cc2C1(C)C]

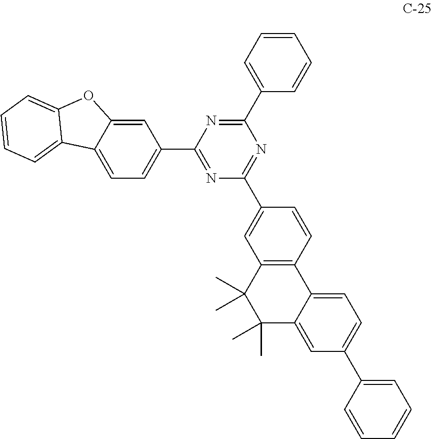 [SMILES: CC1(C)c2cc(-c3ccccc3)ccc2-c2ccc(-c3nc(-c4ccccc4)nc(-c4ccc5c(c4)oc4ccccc45)n3)cc2C1(C)C]

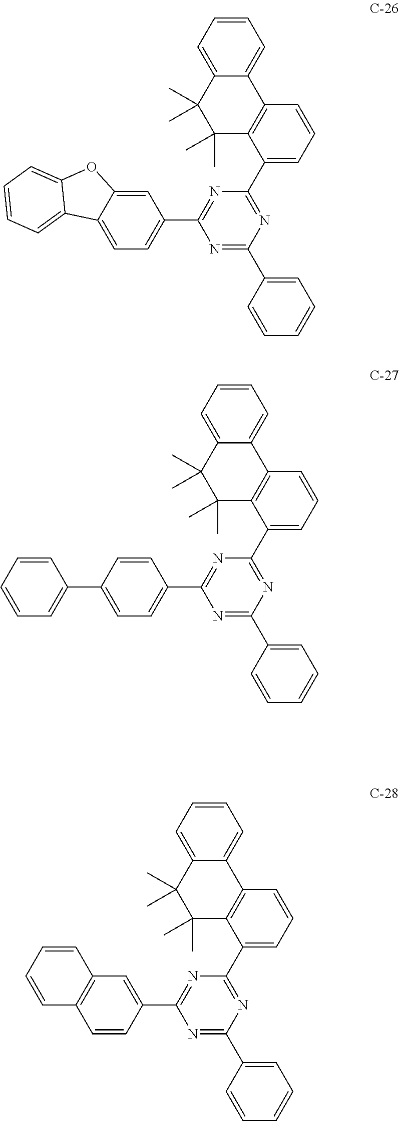 [SMILES: CC1(C)c2ccccc2-c2cccc(-c3nc(-c4ccccc4)nc(-c4ccc5c(c4)oc4ccccc45)n3)c2C1(C)C ; CC1(C)c2ccccc2-c2cccc(-c3nc(-c4ccccc4)nc(-c4ccc(-c5ccccc5)cc4)n3)c2C1(C)C ; CC1(C)c2ccccc2-c2cccc(-c3nc(-c4ccccc4)nc(-c4ccc5ccccc5c4)n3)c2C1(C)C]

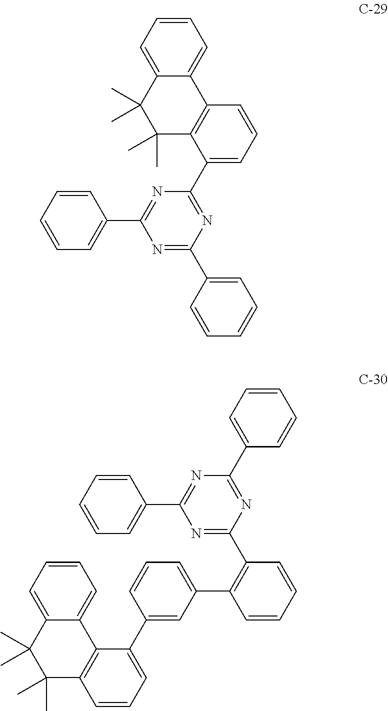 [SMILES: CC1(C)c2ccccc2-c2cccc(-c3nc(-c4ccccc4)nc(-c4ccccc4)n3)c2C1(C)C ; CC1(C)c2ccccc2-c2c(-c3cccc(-c4ccccc4-c4nc(-c5ccccc5)nc(-c5ccccc5)n4)c3)cccc2C1(C)C]

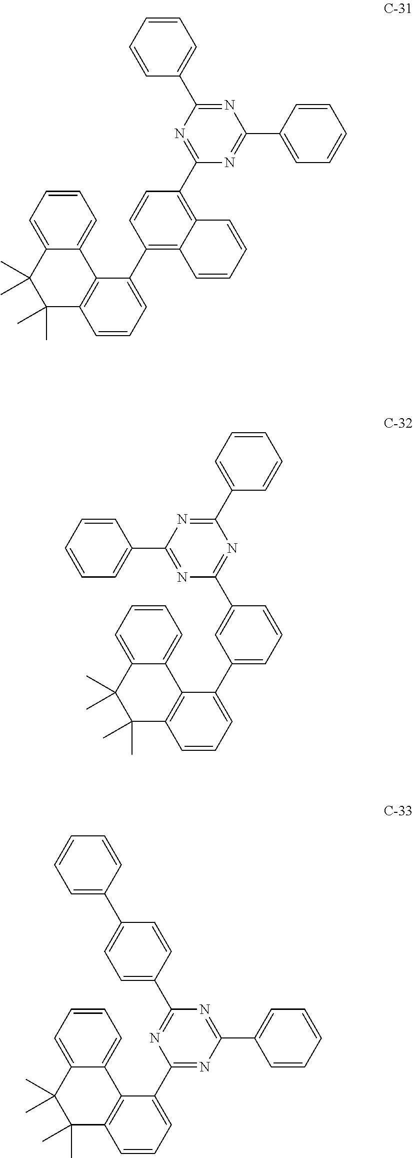 [SMILES: CC1(C)c2ccccc2-c2c(-c3ccc(-c4nc(-c5ccccc5)nc(-c5ccccc5)n4)c4ccccc34)cccc2C1(C)C ; CC1(C)c2ccccc2-c2c(-c3cccc(-c4nc(-c5ccccc5)nc(-c5ccccc5)n4)c3)cccc2C1(C)C ; CC1(C)c2ccccc2-c2c(-c3nc(-c4ccccc4)nc(-c4ccc(-c5ccccc5)cc4)n3)cccc2C1(C)C]

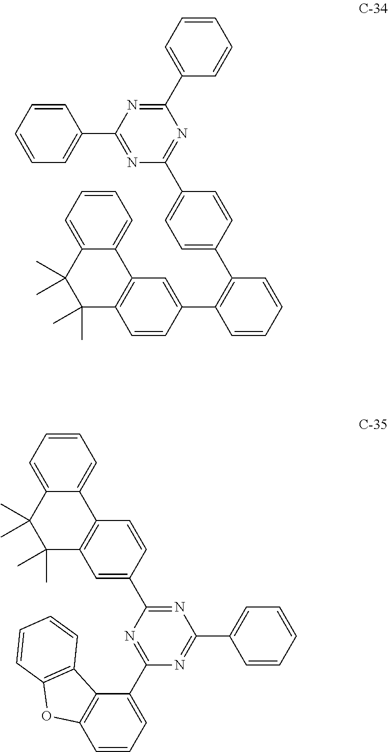 [SMILES: CC1(C)c2ccccc2-c2cc(-c3ccccc3-c3ccc(-c4nc(-c5ccccc5)nc(-c5ccccc5)n4)cc3)ccc2C1(C)C ; CC1(C)c2ccccc2-c2ccc(-c3nc(-c4ccccc4)nc(-c4cccc5oc6ccccc6c45)n3)cc2C1(C)C]

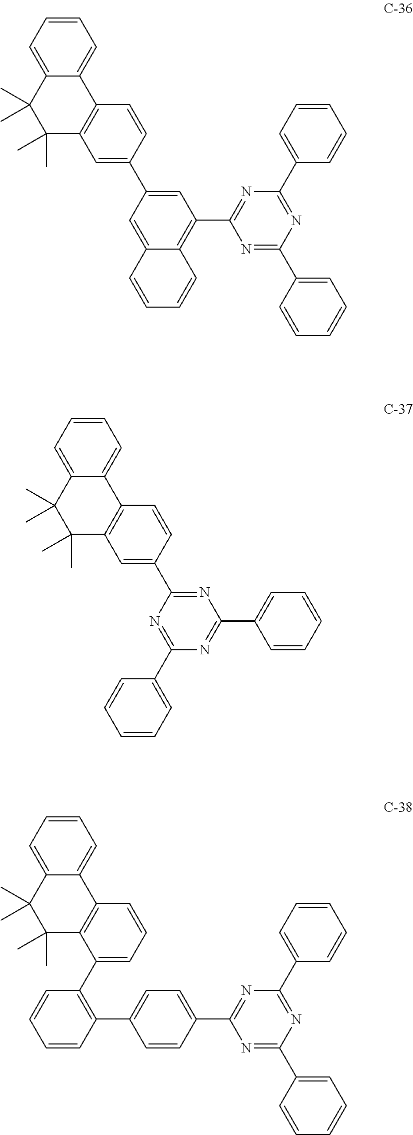 [SMILES: CC1(C)c2ccccc2-c2ccc(-c3cc(-c4nc(-c5ccccc5)nc(-c5ccccc5)n4)c4ccccc4c3)cc2C1(C)C ; CC1(C)c2ccccc2-c2ccc(-c3nc(-c4ccccc4)nc(-c4ccccc4)n3)cc2C1(C)C ; CC1(C)c2ccccc2-c2cccc(-c3ccccc3-c3ccc(-c4nc(-c5ccccc5)nc(-c5ccccc5)n4)cc3)c2C1(C)C]

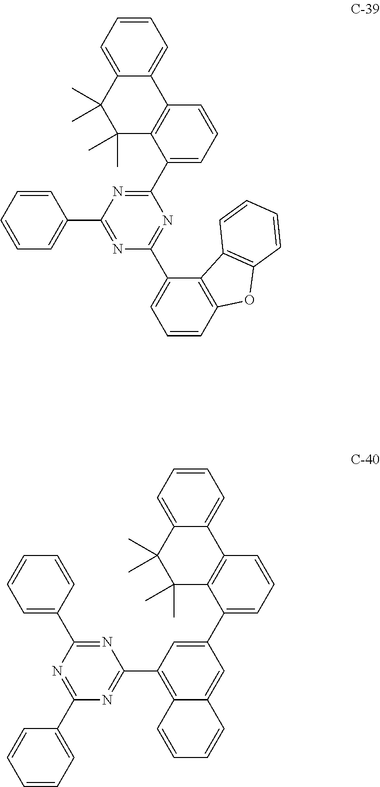 [SMILES: CC1(C)c2ccccc2-c2cccc(-c3nc(-c4ccccc4)nc(-c4cccc5oc6ccccc6c45)n3)c2C1(C)C ; CC1(C)c2ccccc2-c2cccc(-c3cc(-c4nc(-c5ccccc5)nc(-c5ccccc5)n4)c4ccccc4c3)c2C1(C)C]

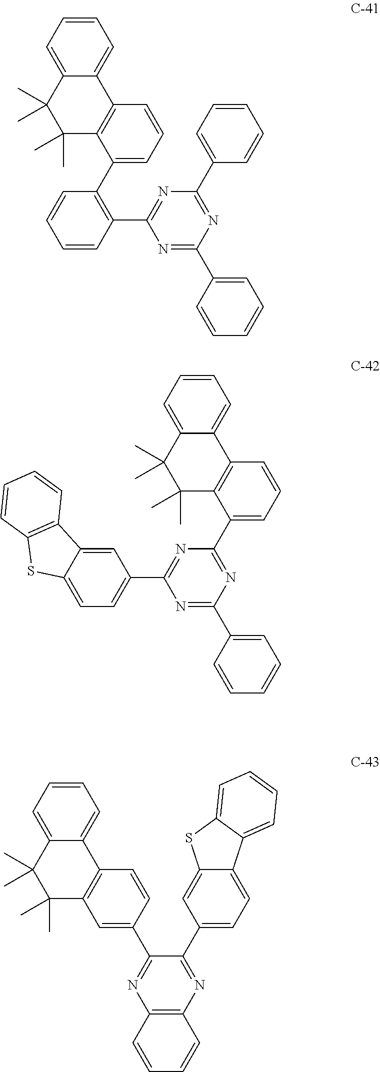

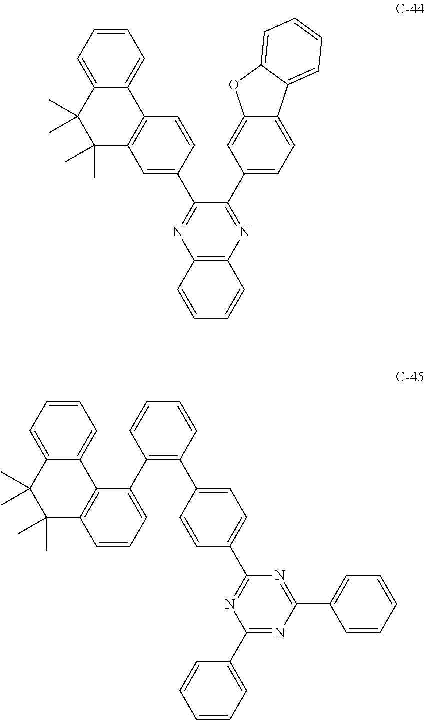 [SMILES: CC1(C)c2ccccc2-c2ccc(-c3nc4ccccc4nc3-c3ccc4c(c3)oc3ccccc34)cc2C1(C)C ; CC1(C)c2ccccc2-c2c(-c3ccccc3-c3ccc(-c4nc(-c5ccccc5)nc(-c5ccccc5)n4)cc3)cccc2C1(C)C]

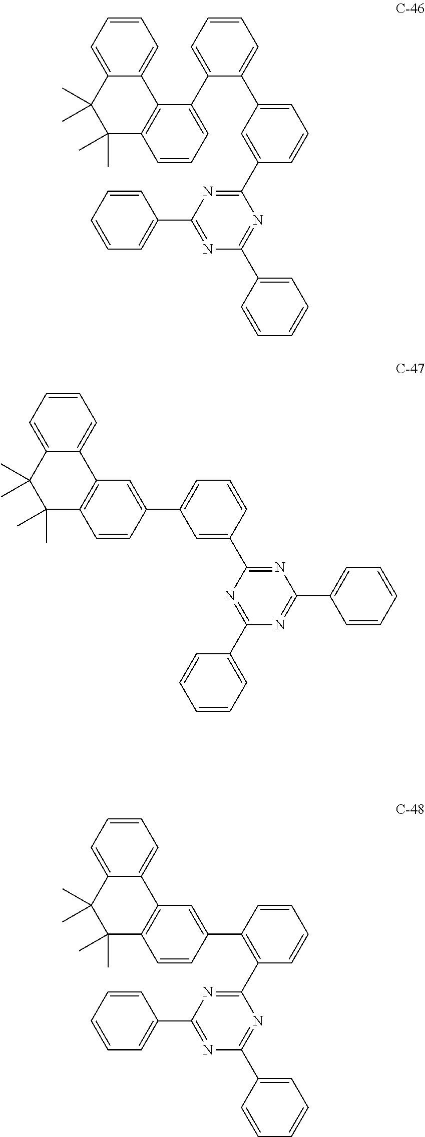 [SMILES: CC1(C)c2ccccc2-c2c(-c3ccccc3-c3cccc(-c4nc(-c5ccccc5)nc(-c5ccccc5)n4)c3)cccc2C1(C)C ; CC1(C)c2ccccc2-c2cc(-c3cccc(-c4nc(-c5ccccc5)nc(-c5ccccc5)n4)c3)ccc2C1(C)C ; CC1(C)c2ccccc2-c2cc(-c3ccccc3-c3nc(-c4ccccc4)nc(-c4ccccc4)n3)ccc2C1(C)C]

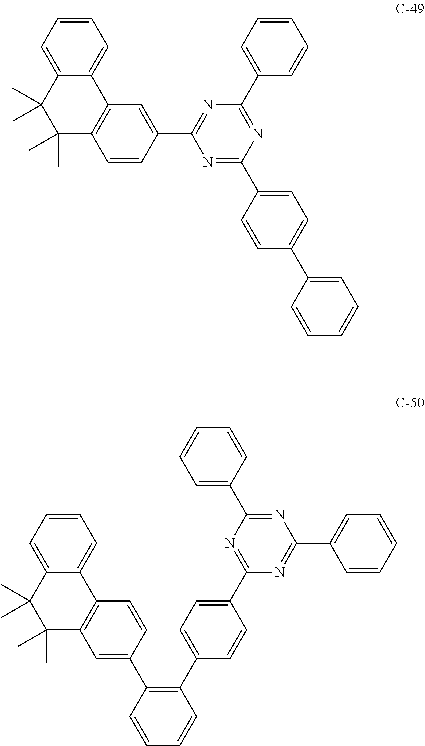 [SMILES: CC1(C)c2ccccc2-c2cc(-c3nc(-c4ccccc4)nc(-c4ccc(-c5ccccc5)cc4)n3)ccc2C1(C)C ; CC1(C)c2ccccc2-c2ccc(-c3ccccc3-c3ccc(-c4nc(-c5ccccc5)nc(-c5ccccc5)n4)cc3)cc2C1(C)C]

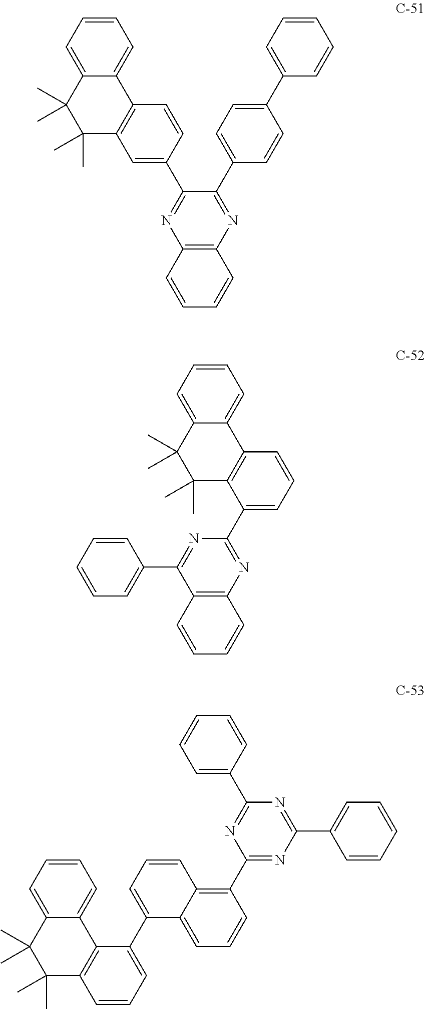 [SMILES: CC1(C)c2ccccc2-c2ccc(-c3nc4ccccc4nc3-c3ccc(-c4ccccc4)cc3)cc2C1(C)C ; CC1(C)c2ccccc2-c2cccc(-c3nc(-c4ccccc4)c4ccccc4n3)c2C1(C)C ; CC1(C)c2ccccc2-c2c(-c3cccc4c(-c5nc(-c6ccccc6)nc(-c6ccccc6)n5)cccc34)cccc2C1(C)C]

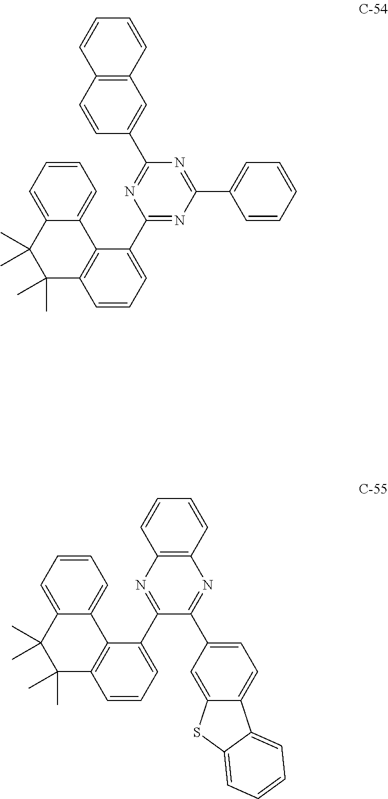 [SMILES: CC1(C)c2ccccc2-c2c(-c3nc(-c4ccccc4)nc(-c4ccc5ccccc5c4)n3)cccc2C1(C)C ; CC1(C)c2ccccc2-c2c(-c3nc4ccccc4nc3-c3ccc4c(c3)sc3ccccc34)cccc2C1(C)C]

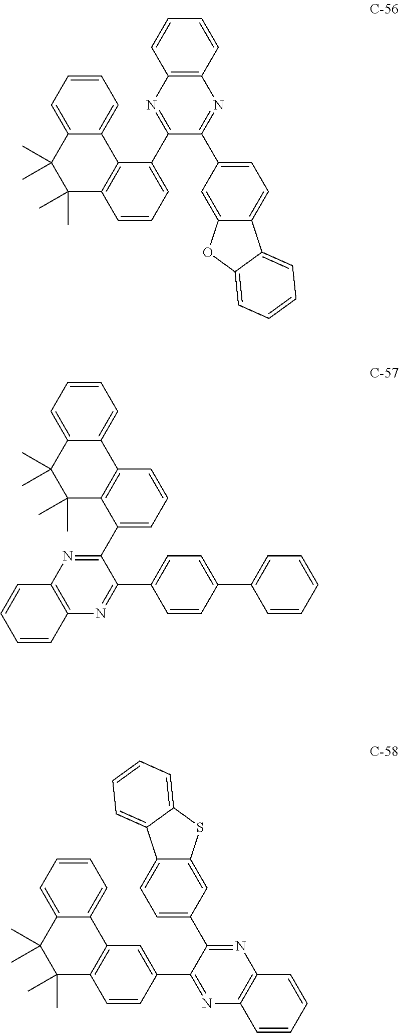 [SMILES: CC1(C)c2ccccc2-c2c(-c3nc4ccccc4nc3-c3ccc4c(c3)oc3ccccc34)cccc2C1(C)C ; CC1(C)c2ccccc2-c2cccc(-c3nc4ccccc4nc3-c3ccc(-c4ccccc4)cc3)c2C1(C)C ; CC1(C)c2ccccc2-c2cc(-c3nc4ccccc4nc3-c3ccc4c(c3)sc3ccccc34)ccc2C1(C)C]

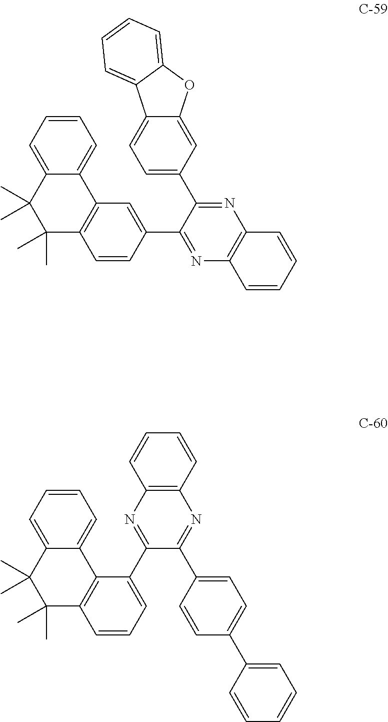 [SMILES: CC1(C)c2ccccc2-c2cc(-c3nc4ccccc4nc3-c3ccc4c(c3)oc3ccccc34)ccc2C1(C)C ; CC1(C)c2ccccc2-c2c(-c3nc4ccccc4nc3-c3ccc(-c4ccccc4)cc3)cccc2C1(C)C]

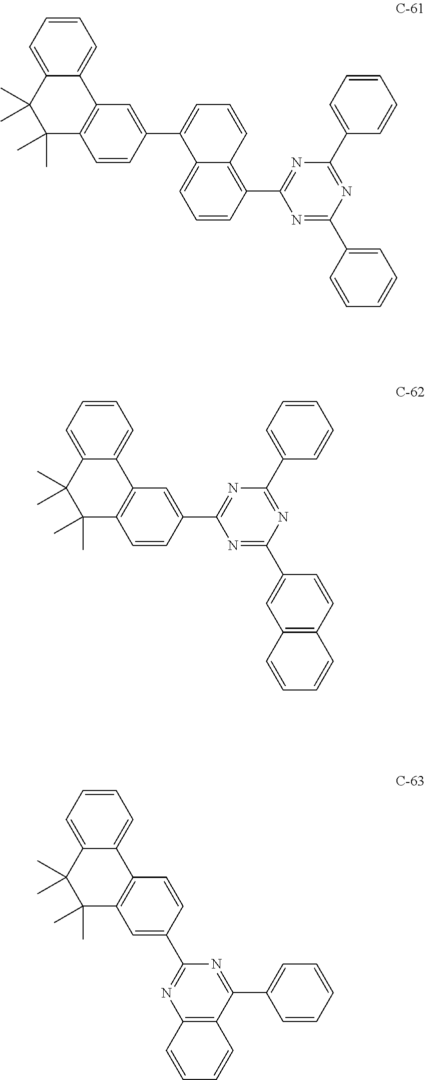 [SMILES: CC1(C)c2ccccc2-c2cc(-c3cccc4c(-c5nc(-c6ccccc6)nc(-c6ccccc6)n5)cccc34)ccc2C1(C)C ; CC1(C)c2ccccc2-c2cc(-c3nc(-c4ccccc4)nc(-c4ccc5ccccc5c4)n3)ccc2C1(C)C ; CC1(C)c2ccccc2-c2ccc(-c3nc(-c4ccccc4)c4ccccc4n3)cc2C1(C)C]

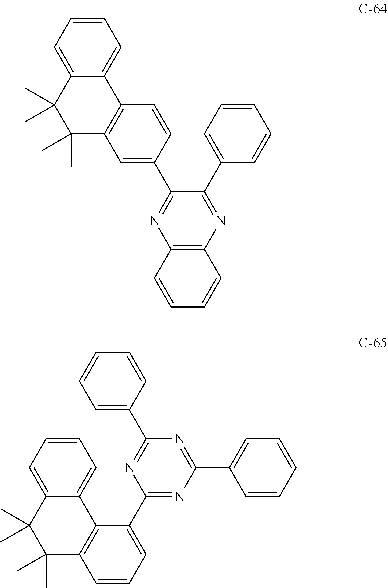 [SMILES: CC1(C)c2ccccc2-c2ccc(-c3nc4ccccc4nc3-c3ccccc3)cc2C1(C)C ; CC1(C)c2ccccc2-c2c(-c3nc(-c4ccccc4)nc(-c4ccccc4)n3)cccc2C1(C)C]

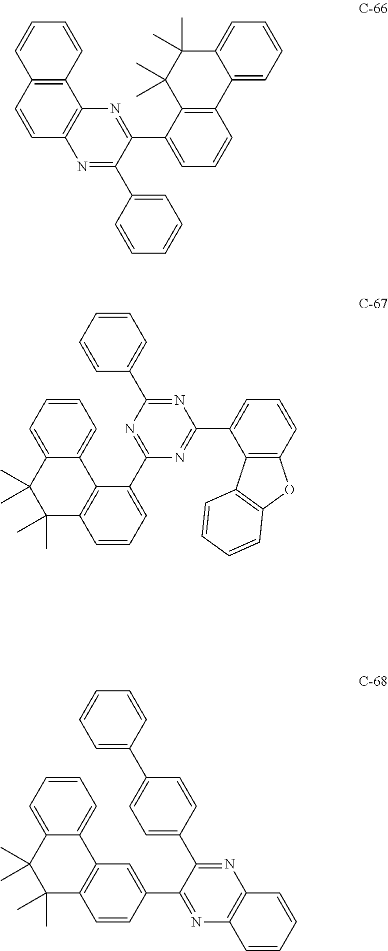 [SMILES: CC1(C)c2ccccc2-c2cccc(-c3nc4c(ccc5ccccc54)nc3-c3ccccc3)c2C1(C)C ; CC1(C)c2ccccc2-c2c(-c3nc(-c4ccccc4)nc(-c4cccc5oc6ccccc6c45)n3)cccc2C1(C)C ; CC1(C)c2ccccc2-c2cc(-c3nc4ccccc4nc3-c3ccc(-c4ccccc4)cc3)ccc2C1(C)C]

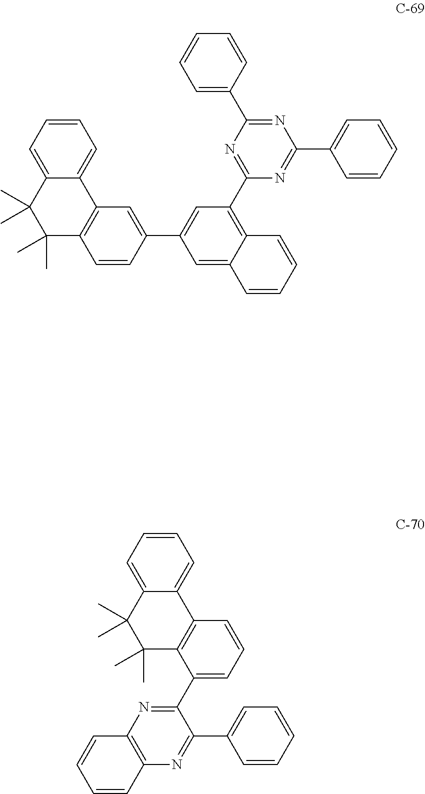 [SMILES: CC1(C)c2ccccc2-c2cc(-c3cc(-c4nc(-c5ccccc5)nc(-c5ccccc5)n4)c4ccccc4c3)ccc2C1(C)C ; CC1(C)c2ccccc2-c2cccc(-c3nc4ccccc4nc3-c3ccccc3)c2C1(C)C]

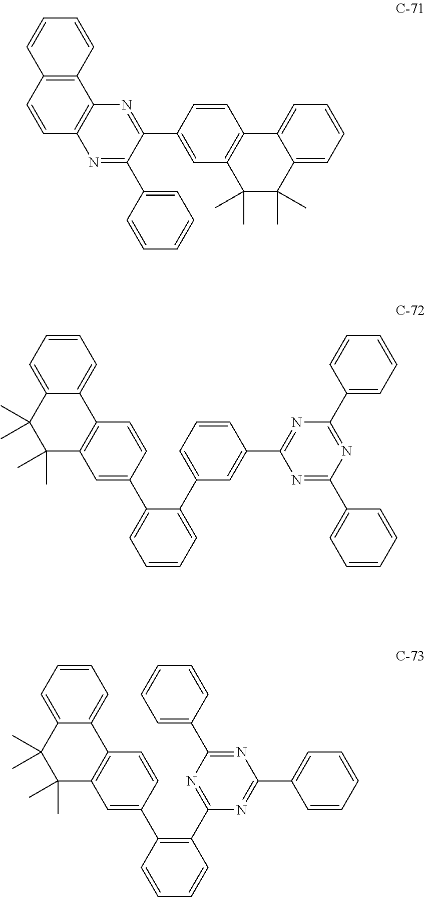 [SMILES: CC1(C)c2ccccc2-c2ccc(-c3nc4c(ccc5ccccc54)nc3-c3ccccc3)cc2C1(C)C ; CC1(C)c2ccccc2-c2ccc(-c3ccccc3-c3cccc(-c4nc(-c5ccccc5)nc(-c5ccccc5)n4)c3)cc2C1(C)C ; CC1(C)c2ccccc2-c2ccc(-c3ccccc3-c3nc(-c4ccccc4)nc(-c4ccccc4)n3)cc2C1(C)C]

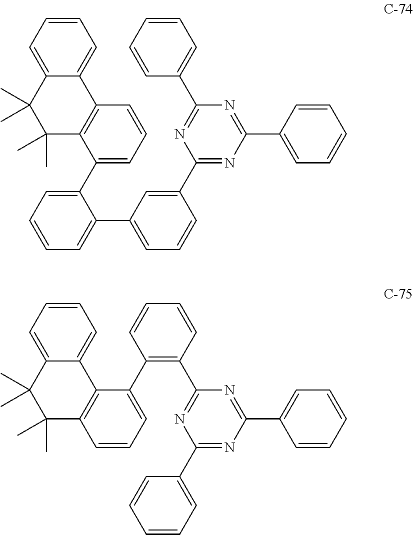 [SMILES: CC1(C)c2ccccc2-c2cccc(-c3ccccc3-c3cccc(-c4nc(-c5ccccc5)nc(-c5ccccc5)n4)c3)c2C1(C)C ; CC1(C)c2ccccc2-c2c(-c3ccccc3-c3nc(-c4ccccc4)nc(-c4ccccc4)n3)cccc2C1(C)C]

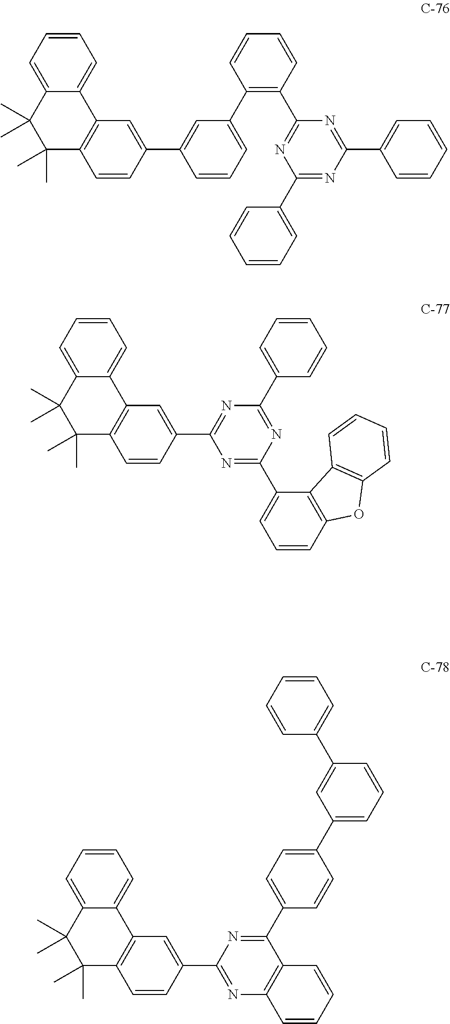 [SMILES: CC1(C)c2ccccc2-c2cc(-c3cccc(-c4ccccc4-c4nc(-c5ccccc5)nc(-c5ccccc5)n4)c3)ccc2C1(C)C ; CC1(C)c2ccccc2-c2cc(-c3nc(-c4ccccc4)nc(-c4cccc5oc6ccccc6c45)n3)ccc2C1(C)C ; CC1(C)c2ccccc2-c2cc(-c3nc(-c4ccc(-c5cccc(-c6ccccc6)c5)cc4)c4ccccc4n3)ccc2C1(C)C]

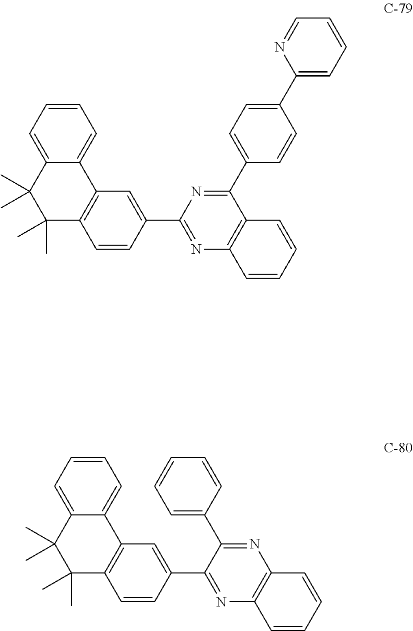 [SMILES: CC1(C)c2ccccc2-c2cc(-c3nc(-c4ccc(-c5ccccn5)cc4)c4ccccc4n3)ccc2C1(C)C ; CC1(C)c2ccccc2-c2cc(-c3nc4ccccc4nc3-c3ccccc3)ccc2C1(C)C]

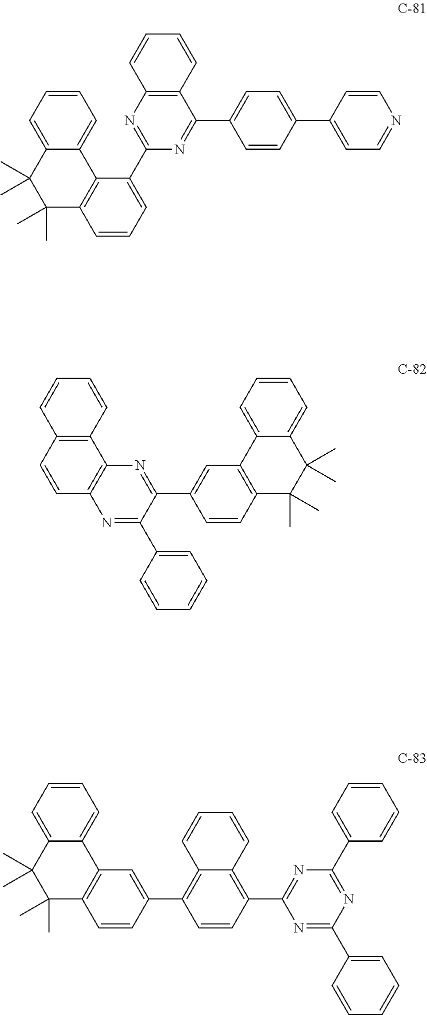

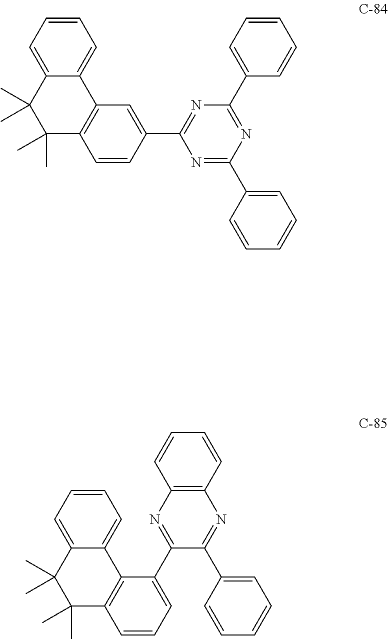 [SMILES: CC1(C)c2ccccc2-c2cc(-c3nc(-c4ccccc4)nc(-c4ccccc4)n3)ccc2C1(C)C ; CC1(C)c2ccccc2-c2c(-c3nc4ccccc4nc3-c3ccccc3)cccc2C1(C)C]

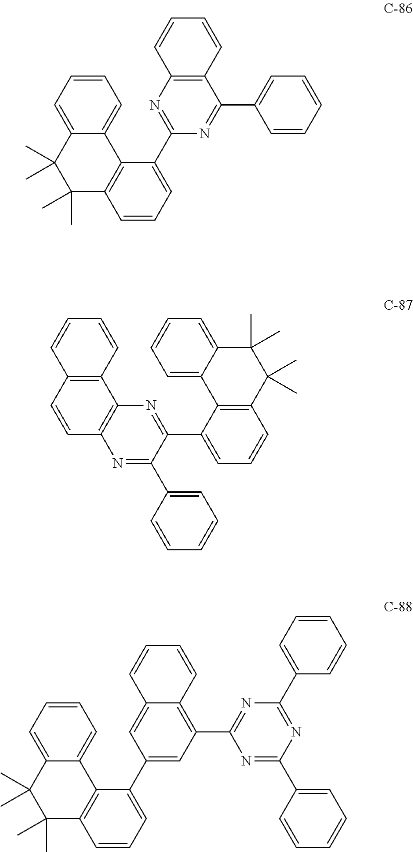

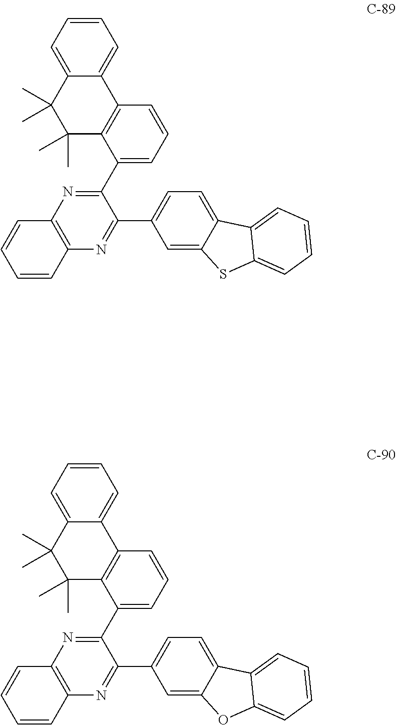 [SMILES: CC1(C)c2ccccc2-c2cccc(-c3nc4ccccc4nc3-c3ccc4c(c3)sc3ccccc34)c2C1(C)C ; CC1(C)c2ccccc2-c2cccc(-c3nc4ccccc4nc3-c3ccc4c(c3)oc3ccccc34)c2C1(C)C]

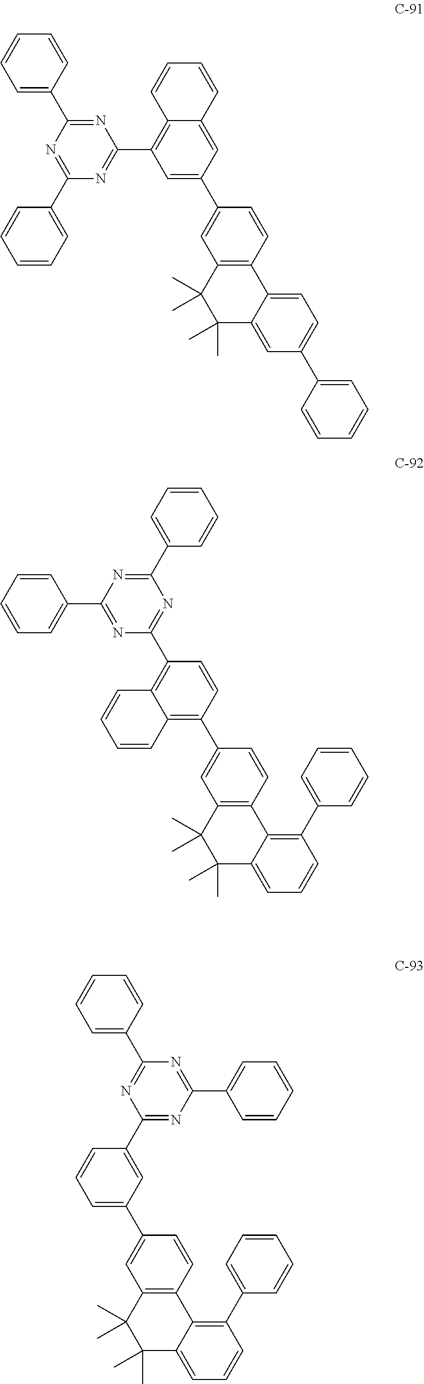 [SMILES: CC1(C)c2cc(-c3ccccc3)ccc2-c2ccc(-c3cc(-c4nc(-c5ccccc5)nc(-c5ccccc5)n4)c4ccccc4c3)cc2C1(C)C ; CC1(C)c2cc(-c3ccc(-c4nc(-c5ccccc5)nc(-c5ccccc5)n4)c4ccccc34)ccc2-c2c(-c3ccccc3)cccc2C1(C)C ; CC1(C)c2cc(-c3cccc(-c4nc(-c5ccccc5)nc(-c5ccccc5)n4)c3)ccc2-c2c(-c3ccccc3)cccc2C1(C)C]

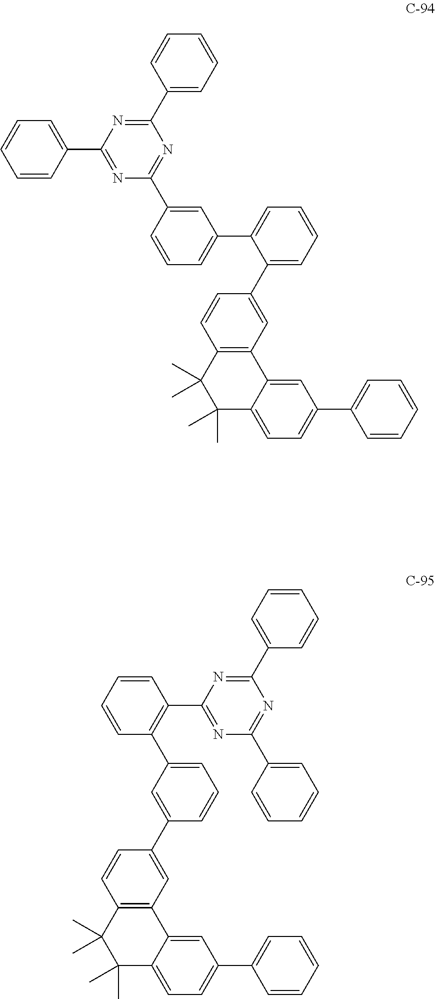 [SMILES: CC1(C)c2ccc(-c3ccccc3)cc2-c2cc(-c3ccccc3-c3cccc(-c4nc(-c5ccccc5)nc(-c5ccccc5)n4)c3)ccc2C1(C)C ; CC1(C)c2ccc(-c3ccccc3)cc2-c2cc(-c3cccc(-c4ccccc4-c4nc(-c5ccccc5)nc(-c5ccccc5)n4)c3)ccc2C1(C)C]

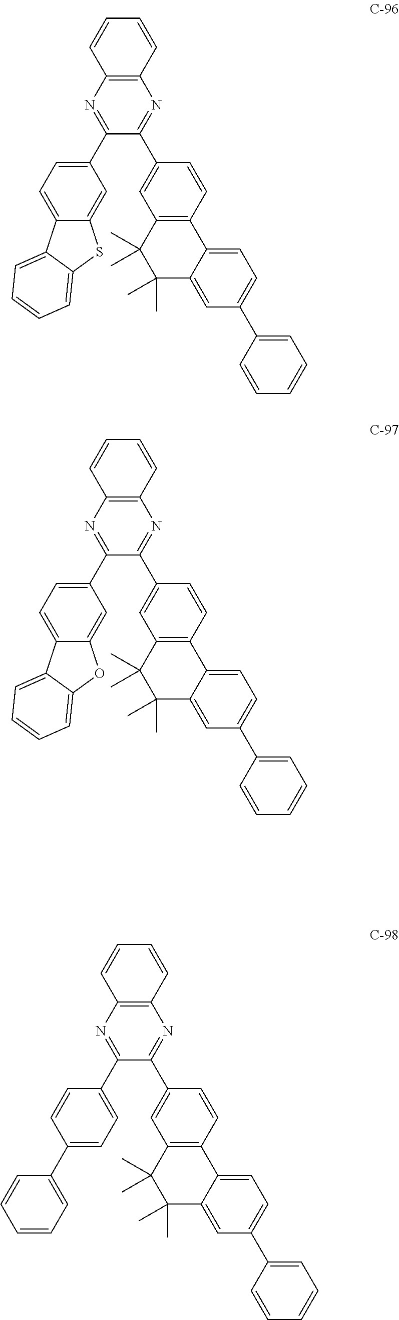 [SMILES: CC1(C)c2cc(-c3ccccc3)ccc2-c2ccc(-c3nc4ccccc4nc3-c3ccc4c(c3)sc3ccccc34)cc2C1(C)C ; CC1(C)c2cc(-c3ccccc3)ccc2-c2ccc(-c3nc4ccccc4nc3-c3ccc4c(c3)oc3ccccc34)cc2C1(C)C ; CC1(C)c2cc(-c3ccccc3)ccc2-c2ccc(-c3nc4ccccc4nc3-c3ccc(-c4ccccc4)cc3)cc2C1(C)C]

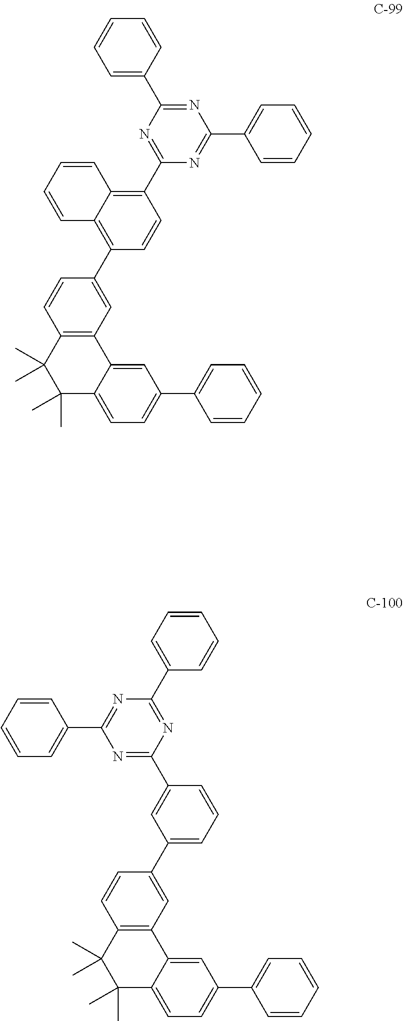 [SMILES: CC1(C)c2ccc(-c3ccccc3)cc2-c2cc(-c3ccc(-c4nc(-c5ccccc5)nc(-c5ccccc5)n4)c4ccccc34)ccc2C1(C)C ; CC1(C)c2ccc(-c3ccccc3)cc2-c2cc(-c3cccc(-c4nc(-c5ccccc5)nc(-c5ccccc5)n4)c3)ccc2C1(C)C]

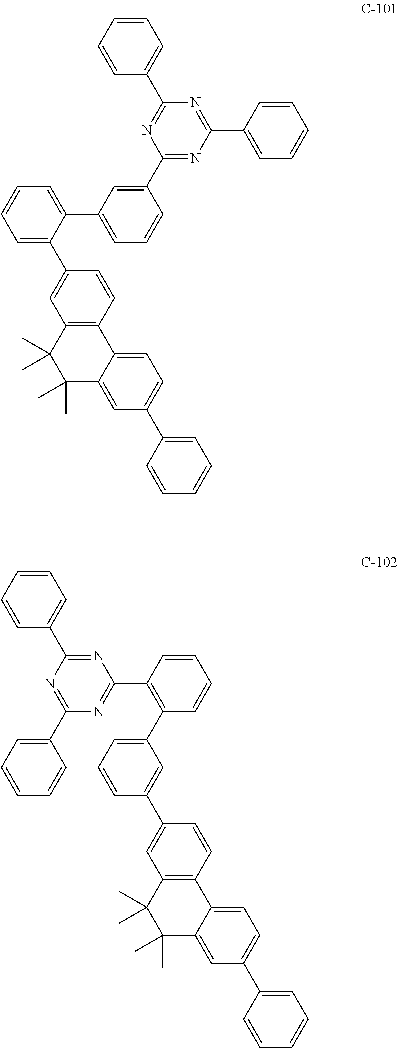 [SMILES: CC1(C)c2cc(-c3ccccc3)ccc2-c2ccc(-c3ccccc3-c3cccc(-c4nc(-c5ccccc5)nc(-c5ccccc5)n4)c3)cc2C1(C)C ; CC1(C)c2cc(-c3ccccc3)ccc2-c2ccc(-c3cccc(-c4ccccc4-c4nc(-c5ccccc5)nc(-c5ccccc5)n4)c3)cc2C1(C)C]

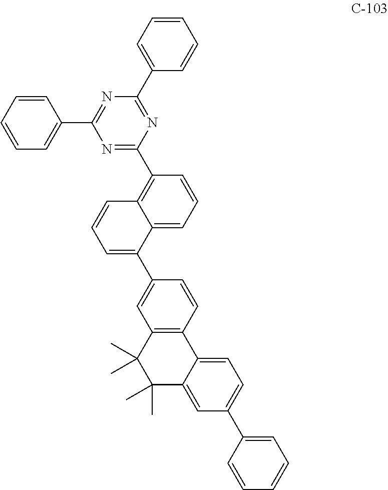 [SMILES: CC1(C)c2cc(-c3ccccc3)ccc2-c2ccc(-c3cccc4c(-c5nc(-c6ccccc6)nc(-c6ccccc6)n5)cccc34)cc2C1(C)C]

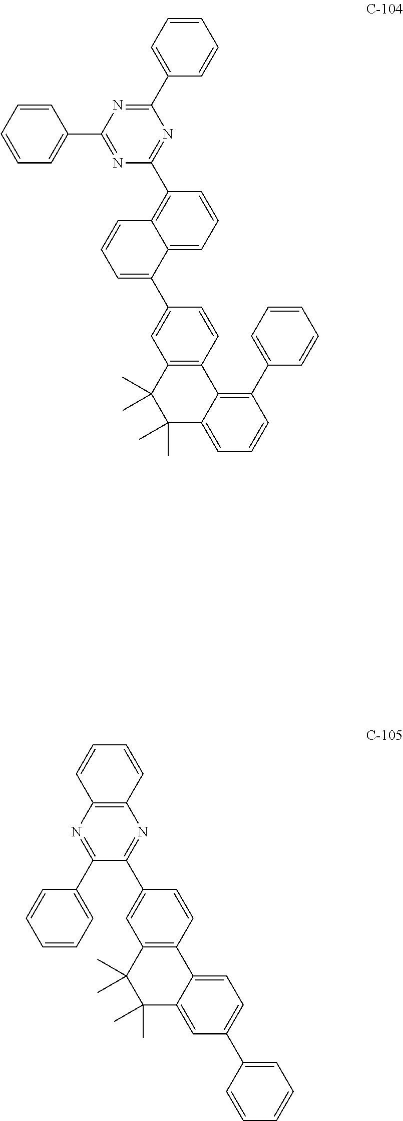 [SMILES: CC1(C)c2cc(-c3cccc4c(-c5nc(-c6ccccc6)nc(-c6ccccc6)n5)cccc34)ccc2-c2c(-c3ccccc3)cccc2C1(C)C ; CC1(C)c2cc(-c3ccccc3)ccc2-c2ccc(-c3nc4ccccc4nc3-c3ccccc3)cc2C1(C)C]

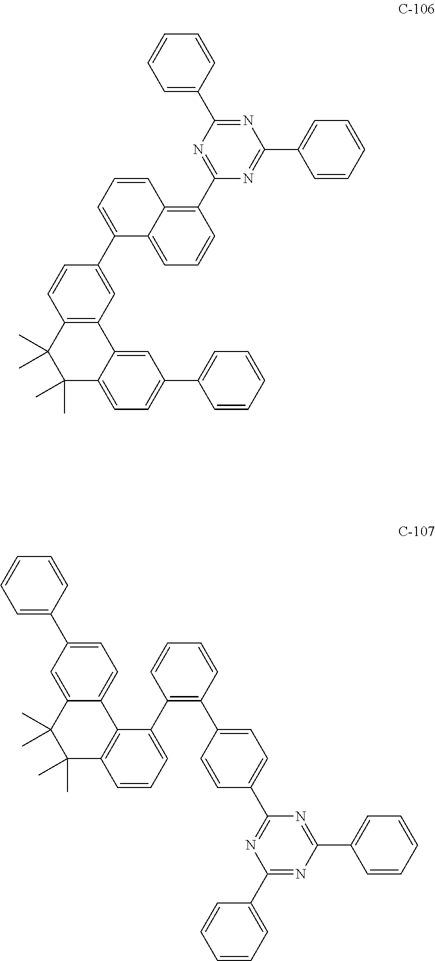 [SMILES: CC1(C)c2ccc(-c3ccccc3)cc2-c2cc(-c3cccc4c(-c5nc(-c6ccccc6)nc(-c6ccccc6)n5)cccc34)ccc2C1(C)C ; CC1(C)c2cc(-c3ccccc3)ccc2-c2c(-c3ccccc3-c3ccc(-c4nc(-c5ccccc5)nc(-c5ccccc5)n4)cc3)cccc2C1(C)C]

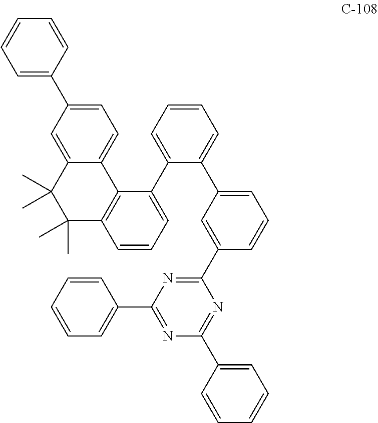 [SMILES: CC1(C)c2cc(-c3ccccc3)ccc2-c2c(-c3ccccc3-c3cccc(-c4nc(-c5ccccc5)nc(-c5ccccc5)n4)c3)cccc2C1(C)C]

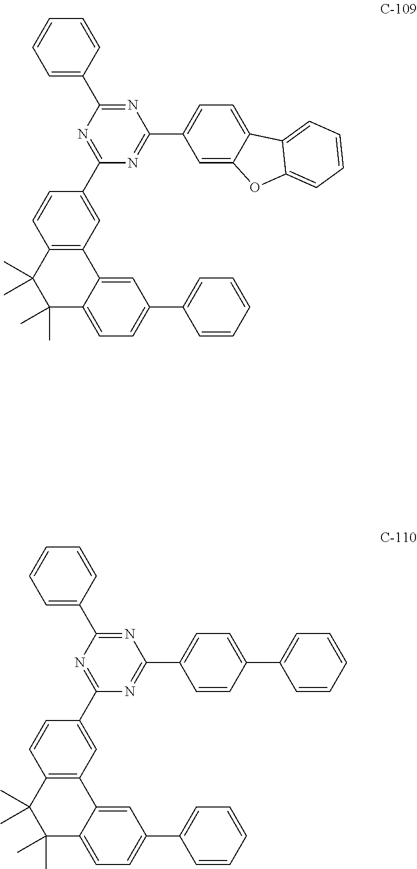 [SMILES: CC1(C)c2ccc(-c3ccccc3)cc2-c2cc(-c3nc(-c4ccccc4)nc(-c4ccc5c(c4)oc4ccccc45)n3)ccc2C1(C)C ; CC1(C)c2ccc(-c3ccccc3)cc2-c2cc(-c3nc(-c4ccccc4)nc(-c4ccc(-c5ccccc5)cc4)n3)ccc2C1(C)C]

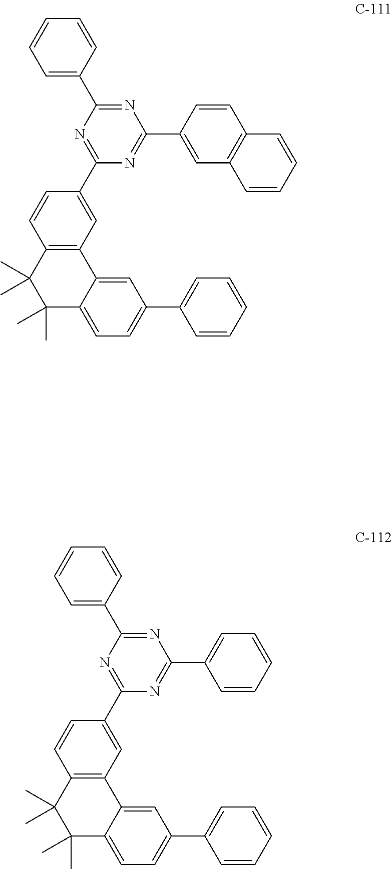 [SMILES: CC1(C)c2ccc(-c3ccccc3)cc2-c2cc(-c3nc(-c4ccccc4)nc(-c4ccc5ccccc5c4)n3)ccc2C1(C)C ; CC1(C)c2ccc(-c3ccccc3)cc2-c2cc(-c3nc(-c4ccccc4)nc(-c4ccccc4)n3)ccc2C1(C)C]

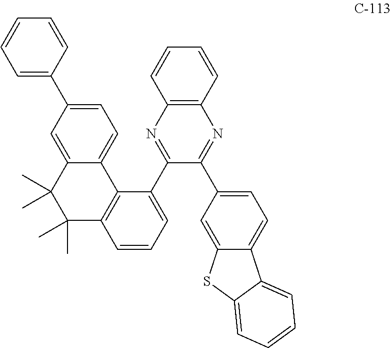 [SMILES: CC1(C)c2cc(-c3ccccc3)ccc2-c2c(-c3nc4ccccc4nc3-c3ccc4c(c3)sc3ccccc34)cccc2C1(C)C]

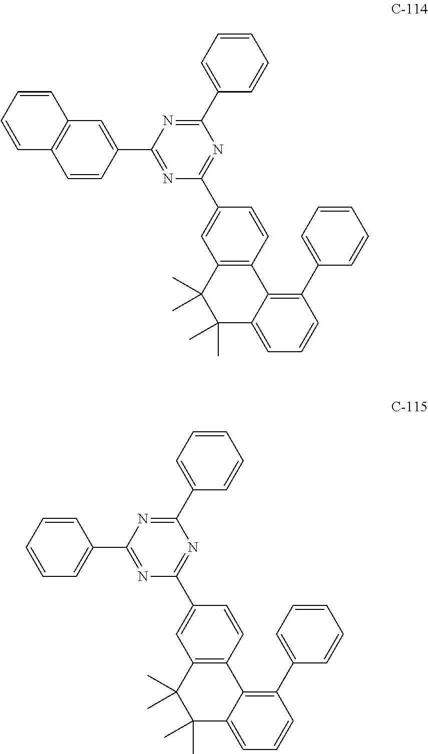 [SMILES: CC1(C)c2cc(-c3nc(-c4ccccc4)nc(-c4ccc5ccccc5c4)n3)ccc2-c2c(-c3ccccc3)cccc2C1(C)C ; CC1(C)c2cc(-c3nc(-c4ccccc4)nc(-c4ccccc4)n3)ccc2-c2c(-c3ccccc3)cccc2C1(C)C]

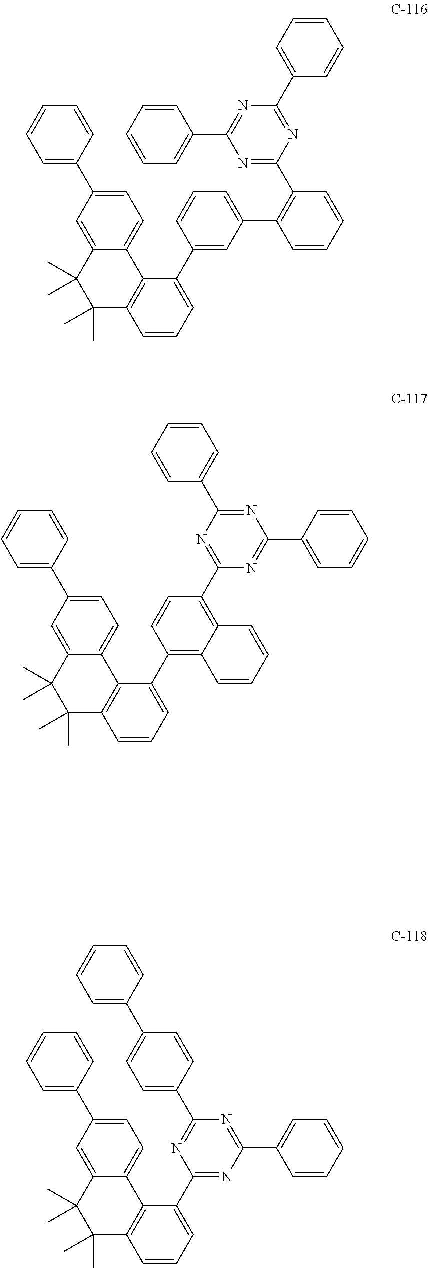 [SMILES: CC1(C)c2cc(-c3ccccc3)ccc2-c2c(-c3cccc(-c4ccccc4-c4nc(-c5ccccc5)nc(-c5ccccc5)n4)c3)cccc2C1(C)C ; CC1(C)c2cc(-c3ccccc3)ccc2-c2c(-c3ccc(-c4nc(-c5ccccc5)nc(-c5ccccc5)n4)c4ccccc34)cccc2C1(C)C ; CC1(C)c2cc(-c3ccccc3)ccc2-c2c(-c3nc(-c4ccccc4)nc(-c4ccc(-c5ccccc5)cc4)n3)cccc2C1(C)C]

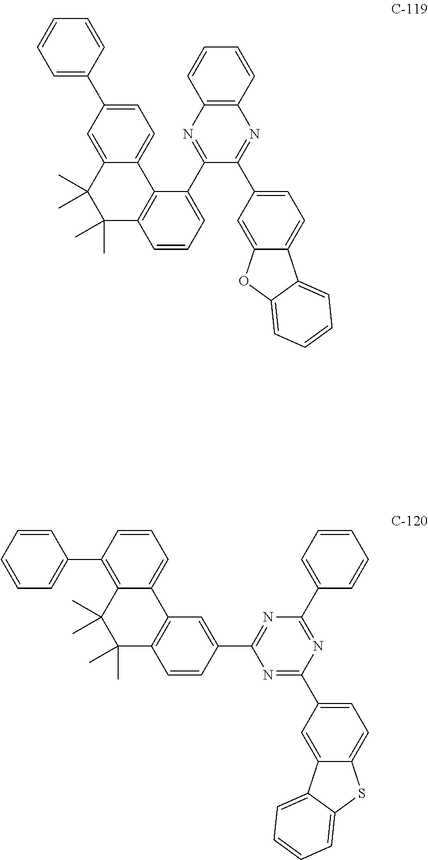 [SMILES: CC1(C)c2cc(-c3ccccc3)ccc2-c2c(-c3nc4ccccc4nc3-c3ccc4c(c3)oc3ccccc34)cccc2C1(C)C ; CC1(C)c2ccc(-c3nc(-c4ccccc4)nc(-c4ccc5sc6ccccc6c5c4)n3)cc2-c2cccc(-c3ccccc3)c2C1(C)C]

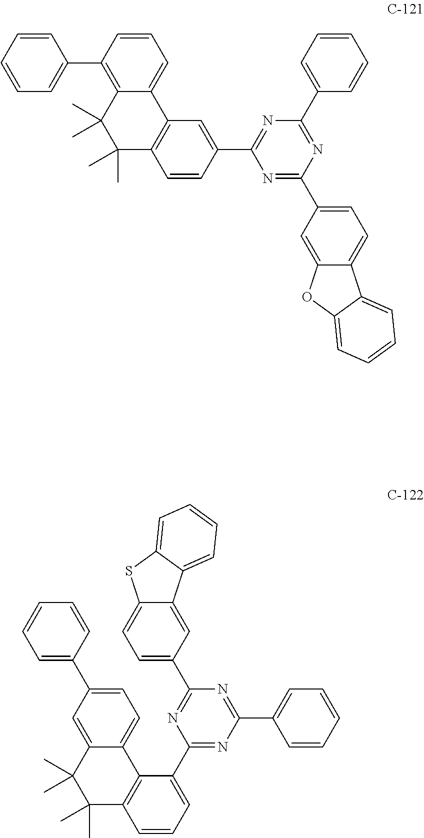 [SMILES: CC1(C)c2ccc(-c3nc(-c4ccccc4)nc(-c4ccc5c(c4)oc4ccccc45)n3)cc2-c2cccc(-c3ccccc3)c2C1(C)C ; CC1(C)c2cc(-c3ccccc3)ccc2-c2c(-c3nc(-c4ccccc4)nc(-c4ccc5sc6ccccc6c5c4)n3)cccc2C1(C)C]

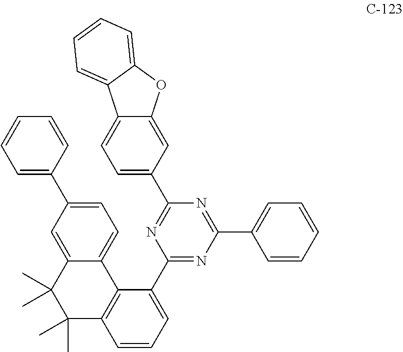 [SMILES: CC1(C)c2cc(-c3ccccc3)ccc2-c2c(-c3nc(-c4ccccc4)nc(-c4ccc5c(c4)oc4ccccc45)n3)cccc2C1(C)C]

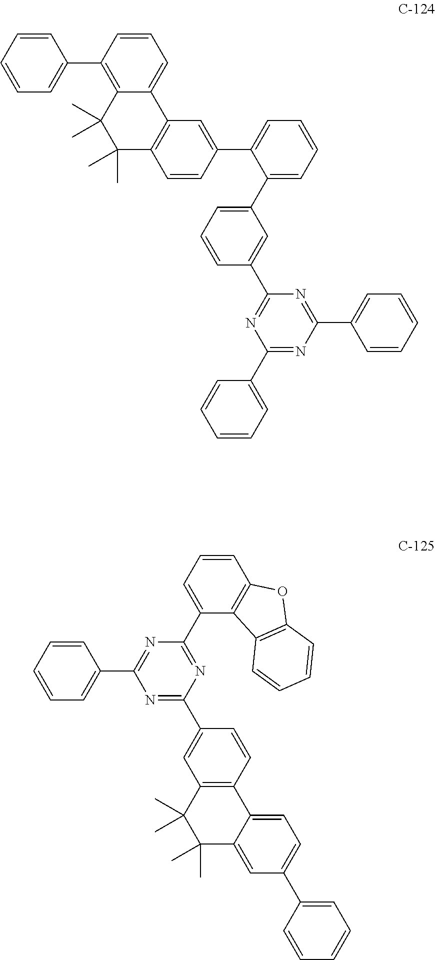 [SMILES: CC1(C)c2ccc(-c3ccccc3-c3cccc(-c4nc(-c5ccccc5)nc(-c5ccccc5)n4)c3)cc2-c2cccc(-c3ccccc3)c2C1(C)C ; CC1(C)c2cc(-c3ccccc3)ccc2-c2ccc(-c3nc(-c4ccccc4)nc(-c4cccc5oc6ccccc6c45)n3)cc2C1(C)C]

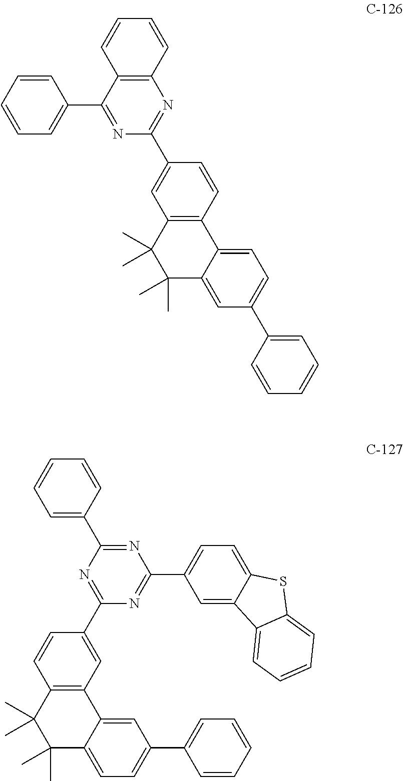 [SMILES: CC1(C)c2cc(-c3ccccc3)ccc2-c2ccc(-c3nc(-c4ccccc4)c4ccccc4n3)cc2C1(C)C ; CC1(C)c2ccc(-c3ccccc3)cc2-c2cc(-c3nc(-c4ccccc4)nc(-c4ccc5sc6ccccc6c5c4)n3)ccc2C1(C)C]

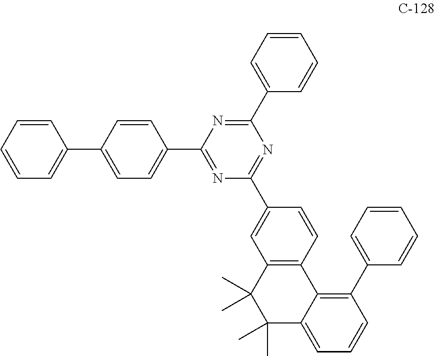 [SMILES: CC1(C)c2cc(-c3nc(-c4ccccc4)nc(-c4ccc(-c5ccccc5)cc4)n3)ccc2-c2c(-c3ccccc3)cccc2C1(C)C]

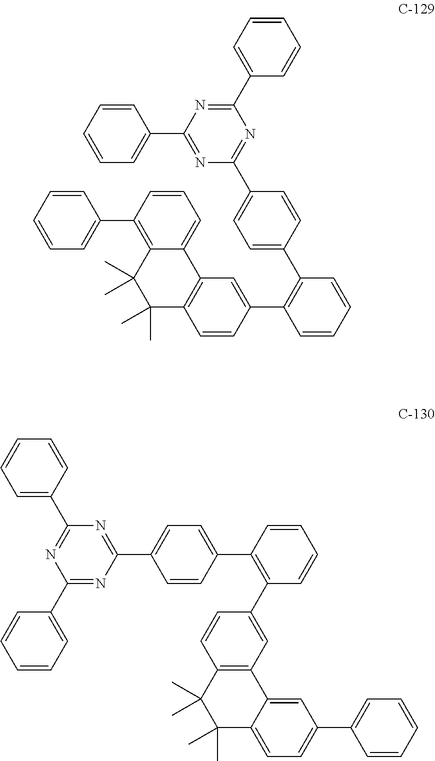 [SMILES: CC1(C)c2ccc(-c3ccccc3-c3ccc(-c4nc(-c5ccccc5)nc(-c5ccccc5)n4)cc3)cc2-c2cccc(-c3ccccc3)c2C1(C)C ; CC1(C)c2ccc(-c3ccccc3)cc2-c2cc(-c3ccccc3-c3ccc(-c4nc(-c5ccccc5)nc(-c5ccccc5)n4)cc3)ccc2C1(C)C]

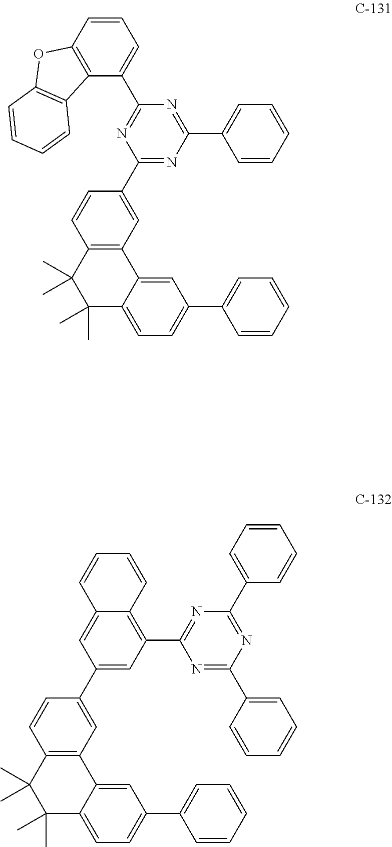 [SMILES: CC1(C)c2ccc(-c3ccccc3)cc2-c2cc(-c3nc(-c4ccccc4)nc(-c4cccc5oc6ccccc6c45)n3)ccc2C1(C)C ; CC1(C)c2ccc(-c3ccccc3)cc2-c2cc(-c3cc(-c4nc(-c5ccccc5)nc(-c5ccccc5)n4)c4ccccc4c3)ccc2C1(C)C]

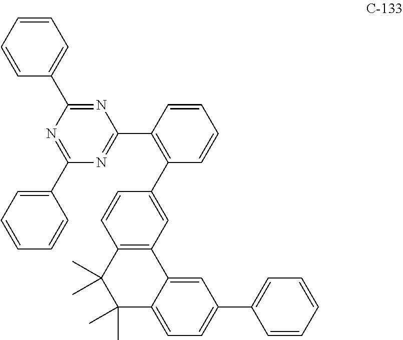 [SMILES: CC1(C)c2ccc(-c3ccccc3)cc2-c2cc(-c3ccccc3-c3nc(-c4ccccc4)nc(-c4ccccc4)n3)ccc2C1(C)C]

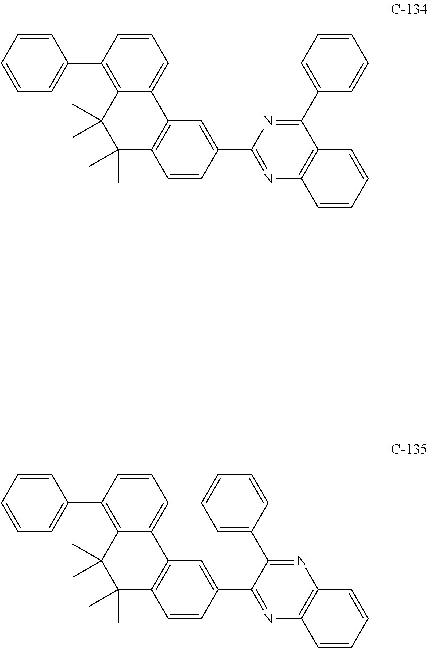 [SMILES: CC1(C)c2ccc(-c3nc(-c4ccccc4)c4ccccc4n3)cc2-c2cccc(-c3ccccc3)c2C1(C)C ; CC1(C)c2ccc(-c3nc4ccccc4nc3-c3ccccc3)cc2-c2cccc(-c3ccccc3)c2C1(C)C]

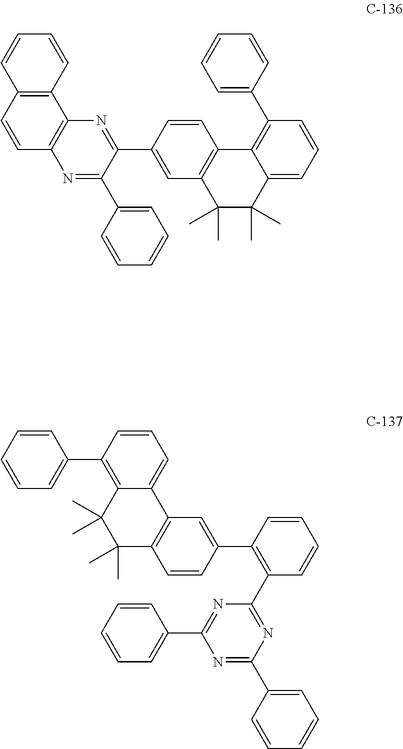 [SMILES: CC1(C)c2cc(-c3nc4c(ccc5ccccc54)nc3-c3ccccc3)ccc2-c2c(-c3ccccc3)cccc2C1(C)C ; CC1(C)c2ccc(-c3ccccc3-c3nc(-c4ccccc4)nc(-c4ccccc4)n3)cc2-c2cccc(-c3ccccc3)c2C1(C)C]

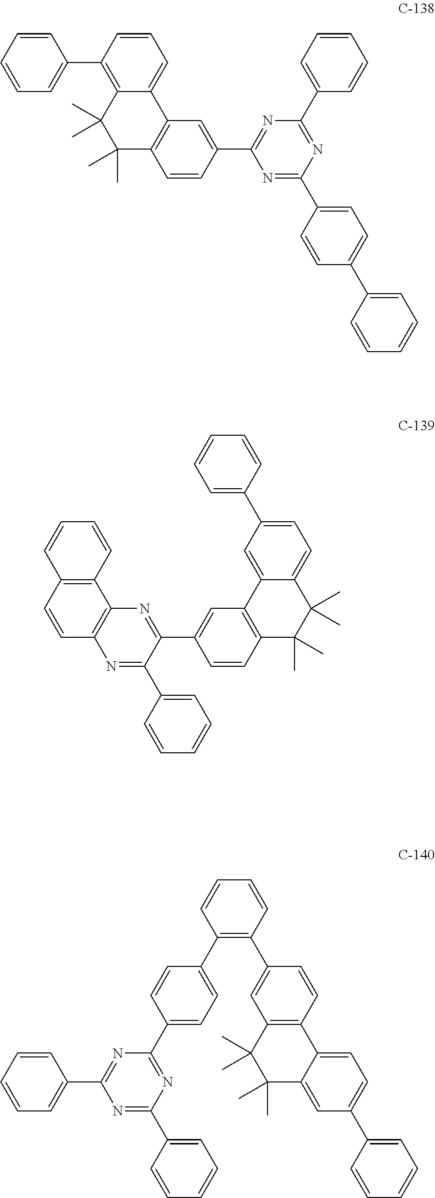 [SMILES: CC1(C)c2ccc(-c3nc(-c4ccccc4)nc(-c4ccc(-c5ccccc5)cc4)n3)cc2-c2cccc(-c3ccccc3)c2C1(C)C ; CC1(C)c2ccc(-c3ccccc3)cc2-c2cc(-c3nc4c(ccc5ccccc54)nc3-c3ccccc3)ccc2C1(C)C ; CC1(C)c2cc(-c3ccccc3)ccc2-c2ccc(-c3ccccc3-c3ccc(-c4nc(-c5ccccc5)nc(-c5ccccc5)n4)cc3)cc2C1(C)C]

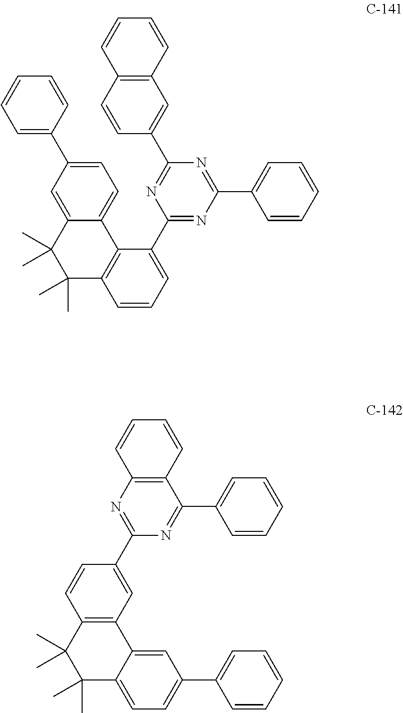 [SMILES: CC1(C)c2cc(-c3ccccc3)ccc2-c2c(-c3nc(-c4ccccc4)nc(-c4ccc5ccccc5c4)n3)cccc2C1(C)C ; CC1(C)c2ccc(-c3ccccc3)cc2-c2cc(-c3nc(-c4ccccc4)c4ccccc4n3)ccc2C1(C)C]

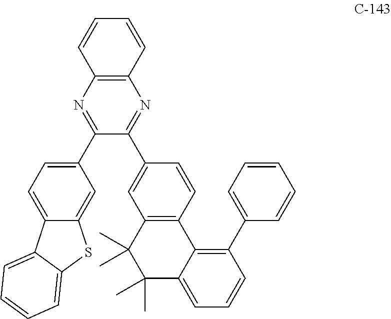 [SMILES: CC1(C)c2cc(-c3nc4ccccc4nc3-c3ccc4c(c3)sc3ccccc34)ccc2-c2c(-c3ccccc3)cccc2C1(C)C]

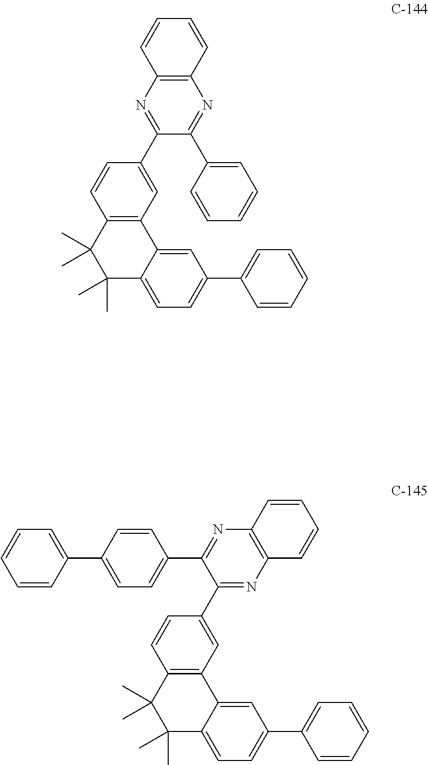 [SMILES: CC1(C)c2ccc(-c3ccccc3)cc2-c2cc(-c3nc4ccccc4nc3-c3ccccc3)ccc2C1(C)C ; CC1(C)c2ccc(-c3ccccc3)cc2-c2cc(-c3nc4ccccc4nc3-c3ccc(-c4ccccc4)cc3)ccc2C1(C)C]

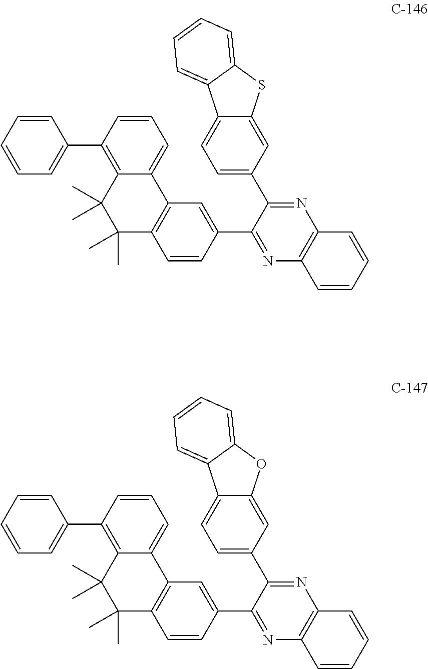 [SMILES: CC1(C)c2ccc(-c3nc4ccccc4nc3-c3ccc4c(c3)sc3ccccc34)cc2-c2cccc(-c3ccccc3)c2C1(C)C ; CC1(C)c2ccc(-c3nc4ccccc4nc3-c3ccc4c(c3)oc3ccccc34)cc2-c2cccc(-c3ccccc3)c2C1(C)C]

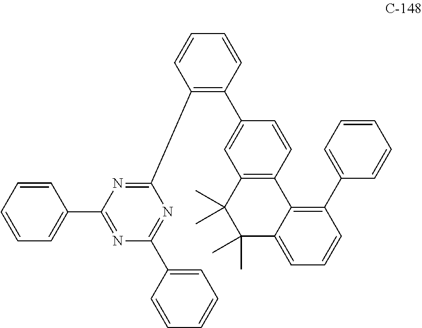 [SMILES: CC1(C)c2cc(-c3ccccc3-c3nc(-c4ccccc4)nc(-c4ccccc4)n3)ccc2-c2c(-c3ccccc3)cccc2C1(C)C]

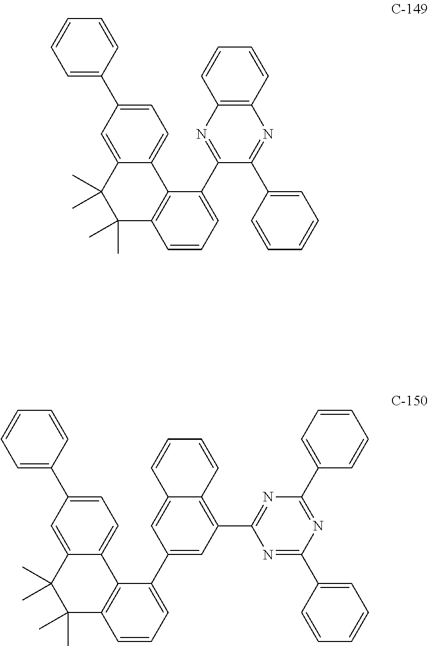 [SMILES: CC1(C)c2cc(-c3ccccc3)ccc2-c2c(-c3nc4ccccc4nc3-c3ccccc3)cccc2C1(C)C ; CC1(C)c2cc(-c3ccccc3)ccc2-c2c(-c3cc(-c4nc(-c5ccccc5)nc(-c5ccccc5)n4)c4ccccc4c3)cccc2C1(C)C]

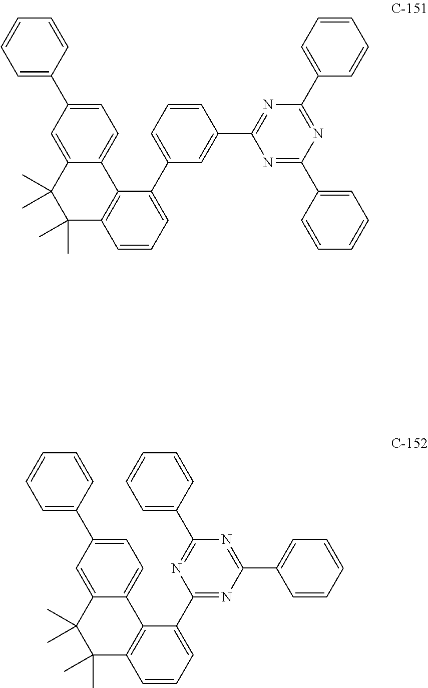 [SMILES: CC1(C)c2cc(-c3ccccc3)ccc2-c2c(-c3cccc(-c4nc(-c5ccccc5)nc(-c5ccccc5)n4)c3)cccc2C1(C)C ; CC1(C)c2cc(-c3ccccc3)ccc2-c2c(-c3nc(-c4ccccc4)nc(-c4ccccc4)n3)cccc2C1(C)C]

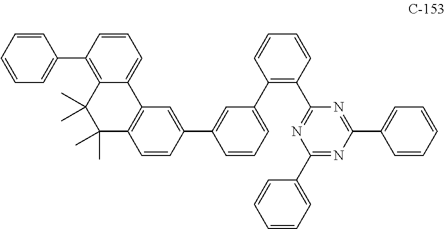 [SMILES: CC1(C)c2ccc(-c3cccc(-c4ccccc4-c4nc(-c5ccccc5)nc(-c5ccccc5)n4)c3)cc2-c2cccc(-c3ccccc3)c2C1(C)C]

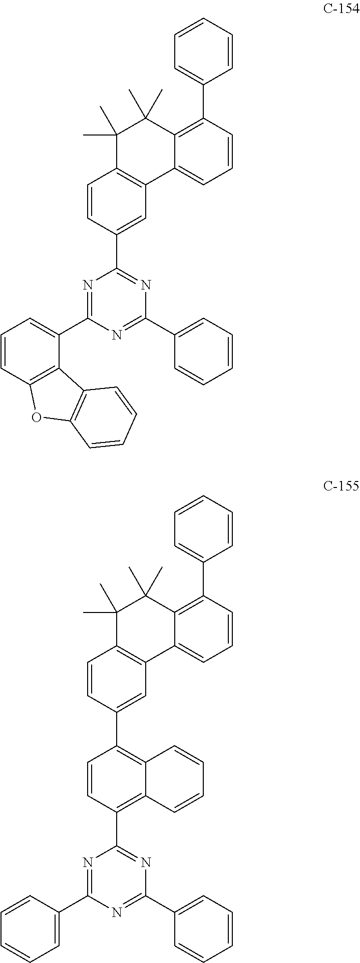 [SMILES: CC1(C)c2ccc(-c3nc(-c4ccccc4)nc(-c4cccc5oc6ccccc6c45)n3)cc2-c2cccc(-c3ccccc3)c2C1(C)C ; CC1(C)c2ccc(-c3ccc(-c4nc(-c5ccccc5)nc(-c5ccccc5)n4)c4ccccc34)cc2-c2cccc(-c3ccccc3)c2C1(C)C]

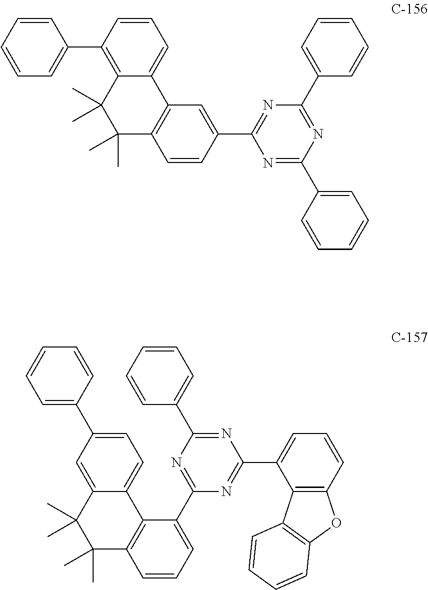 [SMILES: CC1(C)c2ccc(-c3nc(-c4ccccc4)nc(-c4ccccc4)n3)cc2-c2cccc(-c3ccccc3)c2C1(C)C ; CC1(C)c2cc(-c3ccccc3)ccc2-c2c(-c3nc(-c4ccccc4)nc(-c4cccc5oc6ccccc6c45)n3)cccc2C1(C)C]

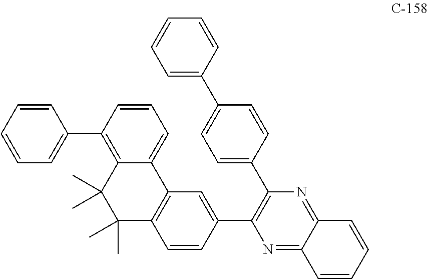 [SMILES: CC1(C)c2ccc(-c3nc4ccccc4nc3-c3ccc(-c4ccccc4)cc3)cc2-c2cccc(-c3ccccc3)c2C1(C)C]

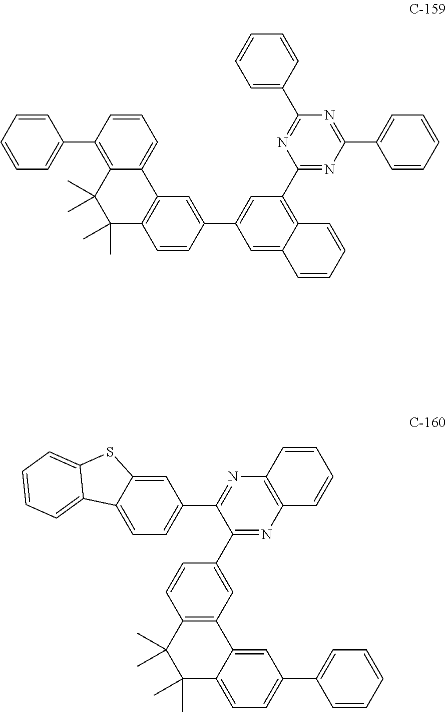 [SMILES: CC1(C)c2ccc(-c3cc(-c4nc(-c5ccccc5)nc(-c5ccccc5)n4)c4ccccc4c3)cc2-c2cccc(-c3ccccc3)c2C1(C)C ; CC1(C)c2ccc(-c3ccccc3)cc2-c2cc(-c3nc4ccccc4nc3-c3ccc4c(c3)sc3ccccc34)ccc2C1(C)C]

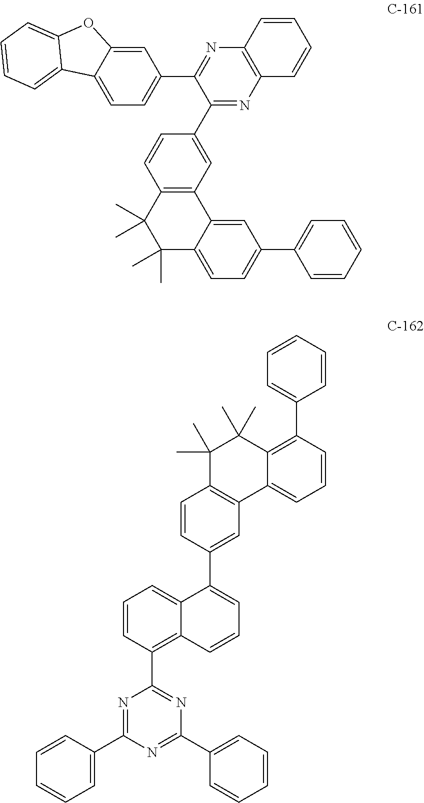 [SMILES: CC1(C)c2ccc(-c3ccccc3)cc2-c2cc(-c3nc4ccccc4nc3-c3ccc4c(c3)oc3ccccc34)ccc2C1(C)C ; CC1(C)c2ccc(-c3cccc4c(-c5nc(-c6ccccc6)nc(-c6ccccc6)n5)cccc34)cc2-c2cccc(-c3ccccc3)c2C1(C)C]

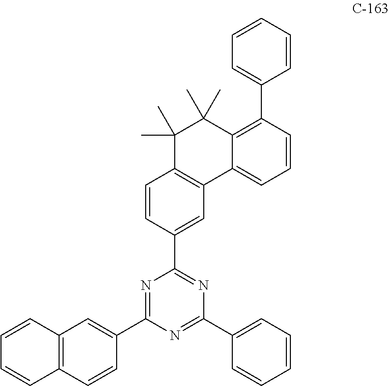 [SMILES: CC1(C)c2ccc(-c3nc(-c4ccccc4)nc(-c4ccc5ccccc5c4)n3)cc2-c2cccc(-c3ccccc3)c2C1(C)C]

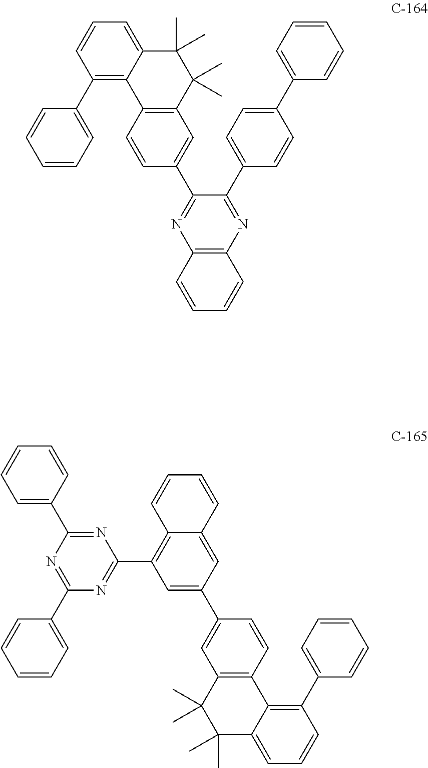 [SMILES: CC1(C)c2cc(-c3nc4ccccc4nc3-c3ccc(-c4ccccc4)cc3)ccc2-c2c(-c3ccccc3)cccc2C1(C)C ; CC1(C)c2cc(-c3cc(-c4nc(-c5ccccc5)nc(-c5ccccc5)n4)c4ccccc4c3)ccc2-c2c(-c3ccccc3)cccc2C1(C)C]

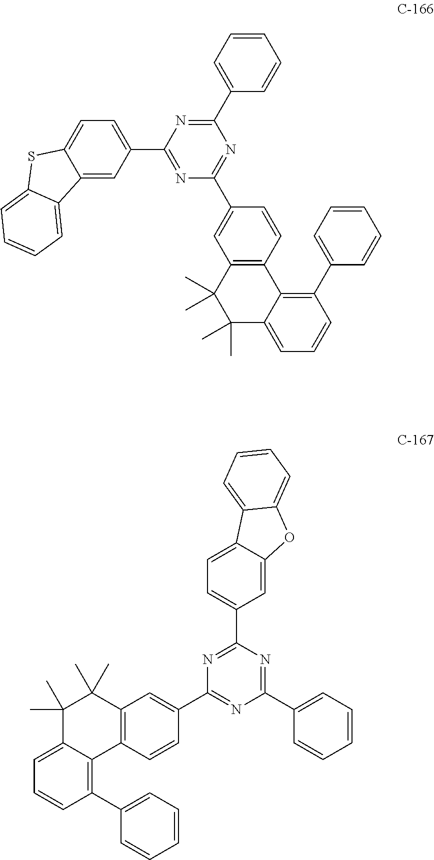 [SMILES: CC1(C)c2cc(-c3nc(-c4ccccc4)nc(-c4ccc5sc6ccccc6c5c4)n3)ccc2-c2c(-c3ccccc3)cccc2C1(C)C ; CC1(C)c2cc(-c3nc(-c4ccccc4)nc(-c4ccc5c(c4)oc4ccccc45)n3)ccc2-c2c(-c3ccccc3)cccc2C1(C)C]

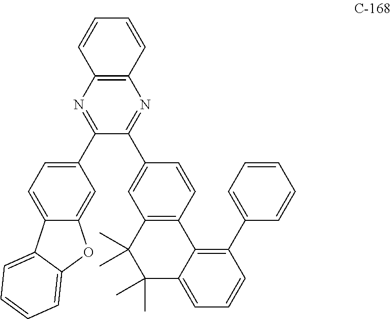 [SMILES: CC1(C)c2cc(-c3nc4ccccc4nc3-c3ccc4c(c3)oc3ccccc34)ccc2-c2c(-c3ccccc3)cccc2C1(C)C]

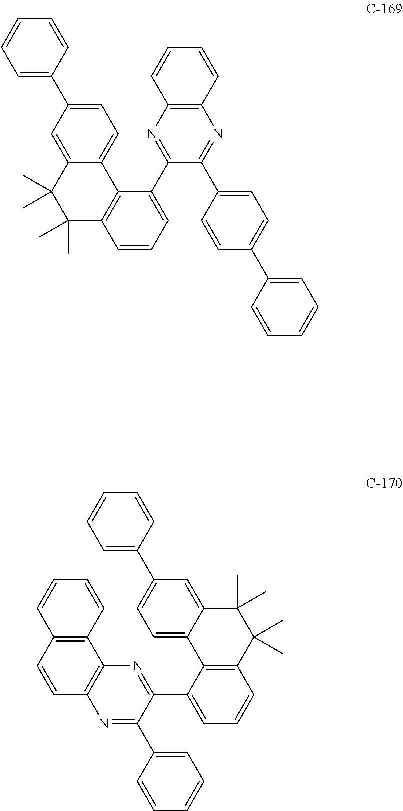 [SMILES: CC1(C)c2cc(-c3ccccc3)ccc2-c2c(-c3nc4ccccc4nc3-c3ccc(-c4ccccc4)cc3)cccc2C1(C)C ; CC1(C)c2cc(-c3ccccc3)ccc2-c2c(-c3nc4c(ccc5ccccc54)nc3-c3ccccc3)cccc2C1(C)C]

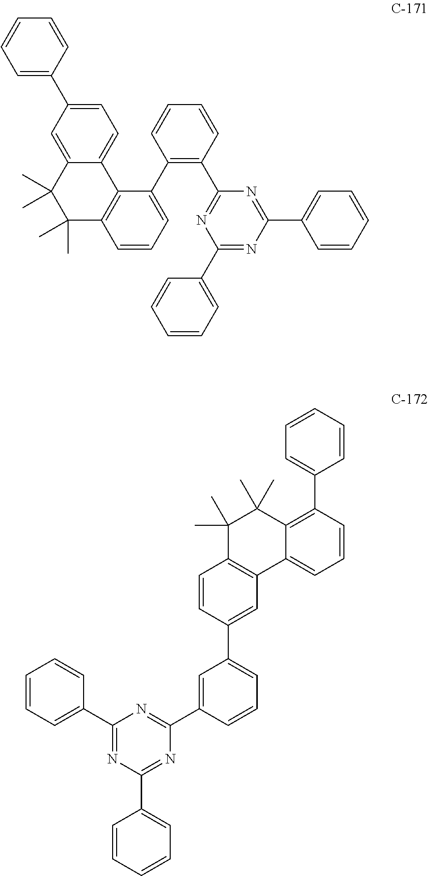 [SMILES: CC1(C)c2cc(-c3ccccc3)ccc2-c2c(-c3ccccc3-c3nc(-c4ccccc4)nc(-c4ccccc4)n3)cccc2C1(C)C ; CC1(C)c2ccc(-c3cccc(-c4nc(-c5ccccc5)nc(-c5ccccc5)n4)c3)cc2-c2cccc(-c3ccccc3)c2C1(C)C]

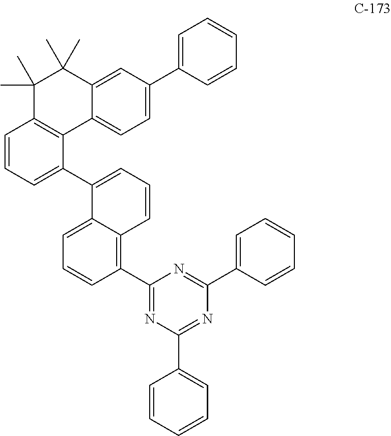 [SMILES: CC1(C)c2cc(-c3ccccc3)ccc2-c2c(-c3cccc4c(-c5nc(-c6ccccc6)nc(-c6ccccc6)n5)cccc34)cccc2C1(C)C]

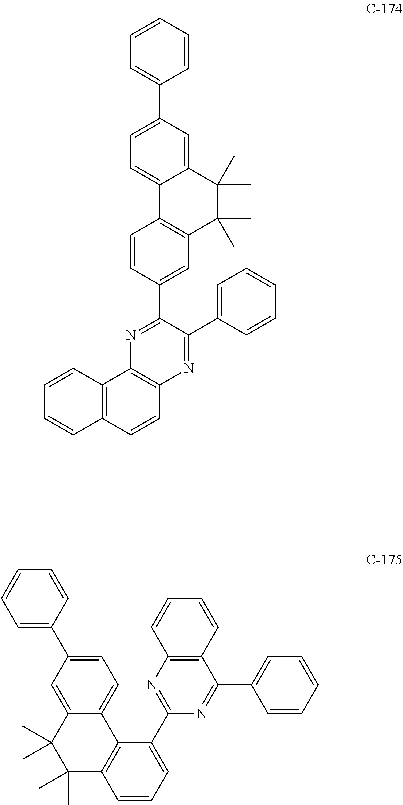 [SMILES: CC1(C)c2cc(-c3ccccc3)ccc2-c2ccc(-c3nc4c(ccc5ccccc54)nc3-c3ccccc3)cc2C1(C)C ; CC1(C)c2cc(-c3ccccc3)ccc2-c2c(-c3nc(-c4ccccc4)c4ccccc4n3)cccc2C1(C)C]

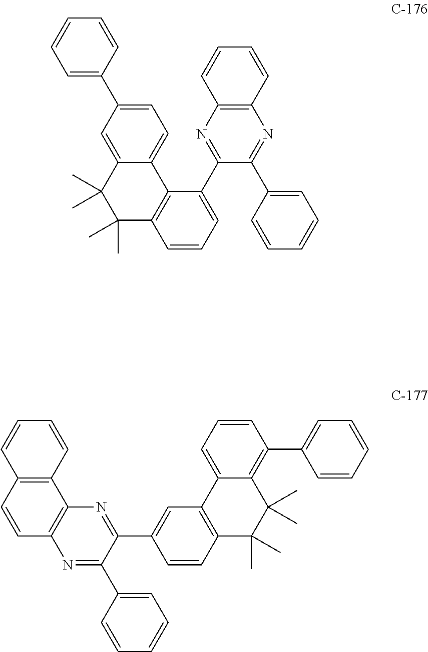 [SMILES: CC1(C)c2cc(-c3ccccc3)ccc2-c2c(-c3nc4ccccc4nc3-c3ccccc3)cccc2C1(C)C ; CC1(C)c2ccc(-c3nc4c(ccc5ccccc54)nc3-c3ccccc3)cc2-c2cccc(-c3ccccc3)c2C1(C)C]

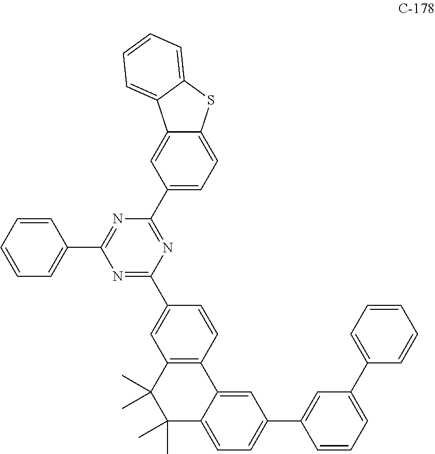 [SMILES: CC1(C)c2ccc(-c3cccc(-c4ccccc4)c3)cc2-c2ccc(-c3nc(-c4ccccc4)nc(-c4ccc5sc6ccccc6c5c4)n3)cc2C1(C)C]

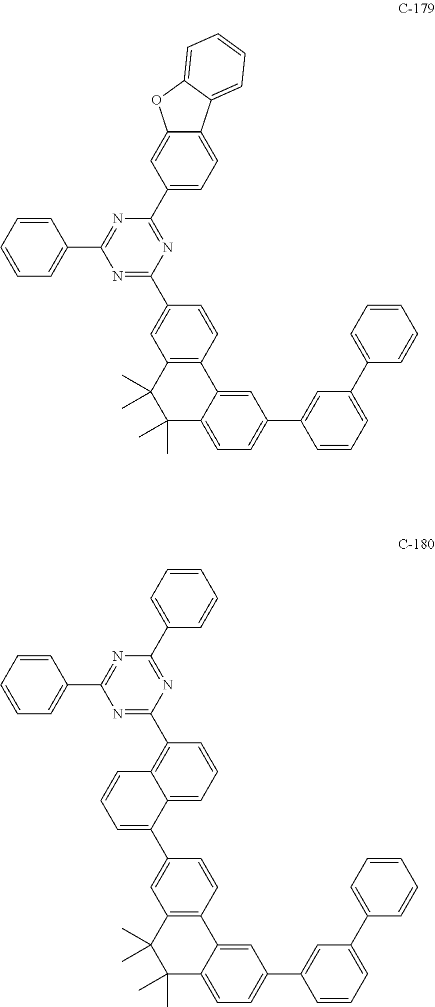 [SMILES: CC1(C)c2ccc(-c3cccc(-c4ccccc4)c3)cc2-c2ccc(-c3nc(-c4ccccc4)nc(-c4ccc5c(c4)oc4ccccc45)n3)cc2C1(C)C ; CC1(C)c2ccc(-c3cccc(-c4ccccc4)c3)cc2-c2ccc(-c3cccc4c(-c5nc(-c6ccccc6)nc(-c6ccccc6)n5)cccc34)cc2C1(C)C]

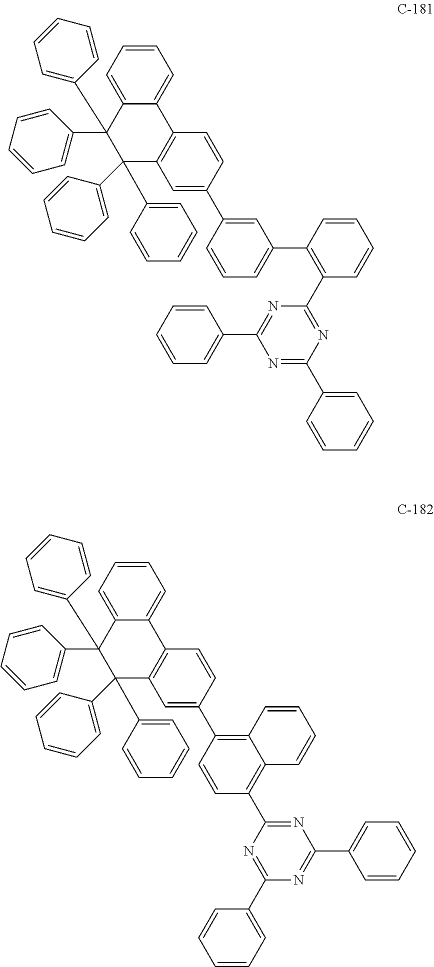 [SMILES: c1ccc(-c2nc(-c3ccccc3)nc(-c3ccccc3-c3cccc(-c4ccc5c(c4)C(c4ccccc4)(c4ccccc4)C(c4ccccc4)(c4ccccc4)c4ccccc4-5)c3)n2)cc1 ; c1ccc(-c2nc(-c3ccccc3)nc(-c3ccc(-c4ccc5c(c4)C(c4ccccc4)(c4ccccc4)C(c4ccccc4)(c4ccccc4)c4ccccc4-5)c4ccccc34)n2)cc1]

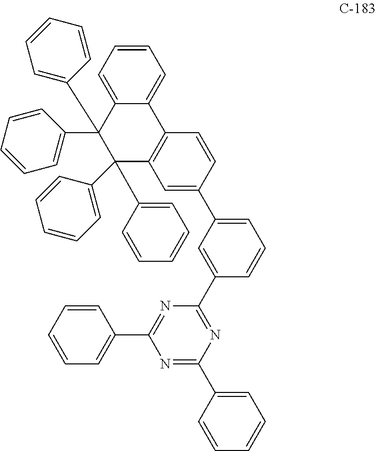 [SMILES: c1ccc(-c2nc(-c3ccccc3)nc(-c3cccc(-c4ccc5c(c4)C(c4ccccc4)(c4ccccc4)C(c4ccccc4)(c4ccccc4)c4ccccc4-5)c3)n2)cc1]

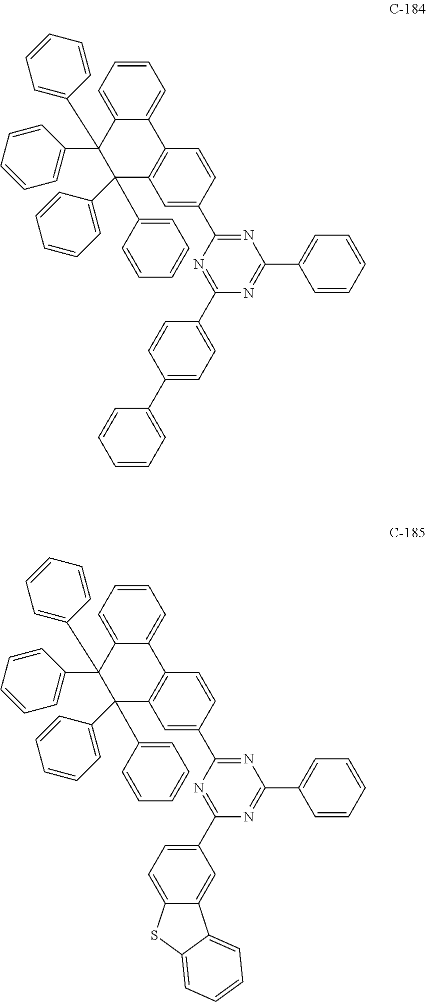 [SMILES: c1ccc(-c2ccc(-c3nc(-c4ccccc4)nc(-c4ccc5c(c4)C(c4ccccc4)(c4ccccc4)C(c4ccccc4)(c4ccccc4)c4ccccc4-5)n3)cc2)cc1 ; c1ccc(-c2nc(-c3ccc4c(c3)C(c3ccccc3)(c3ccccc3)C(c3ccccc3)(c3ccccc3)c3ccccc3-4)nc(-c3ccc4sc5ccccc5c4c3)n2)cc1]

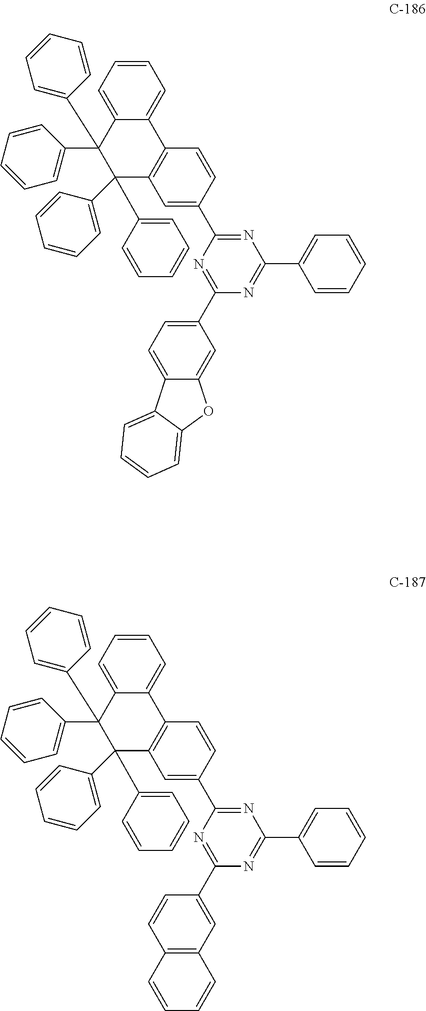 [SMILES: c1ccc(-c2nc(-c3ccc4c(c3)C(c3ccccc3)(c3ccccc3)C(c3ccccc3)(c3ccccc3)c3ccccc3-4)nc(-c3ccc4c(c3)oc3ccccc34)n2)cc1 ; c1ccc(-c2nc(-c3ccc4c(c3)C(c3ccccc3)(c3ccccc3)C(c3ccccc3)(c3ccccc3)c3ccccc3-4)nc(-c3ccc4ccccc4c3)n2)cc1]

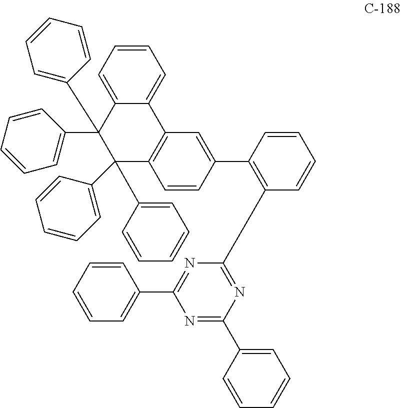 [SMILES: c1ccc(-c2nc(-c3ccccc3)nc(-c3ccccc3-c3ccc4c(c3)-c3ccccc3C(c3ccccc3)(c3ccccc3)C4(c3ccccc3)c3ccccc3)n2)cc1]

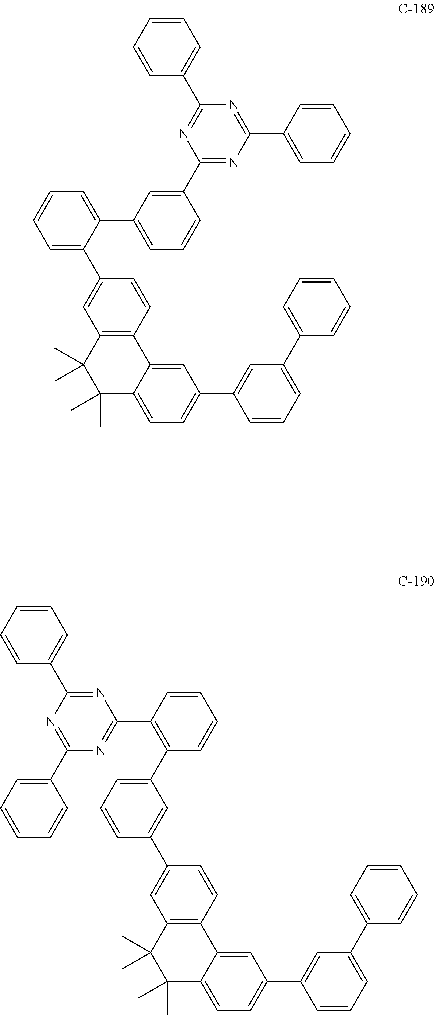 [SMILES: CC1(C)c2ccc(-c3cccc(-c4ccccc4)c3)cc2-c2ccc(-c3ccccc3-c3cccc(-c4nc(-c5ccccc5)nc(-c5ccccc5)n4)c3)cc2C1(C)C ; CC1(C)c2ccc(-c3cccc(-c4ccccc4)c3)cc2-c2ccc(-c3cccc(-c4ccccc4-c4nc(-c5ccccc5)nc(-c5ccccc5)n4)c3)cc2C1(C)C]

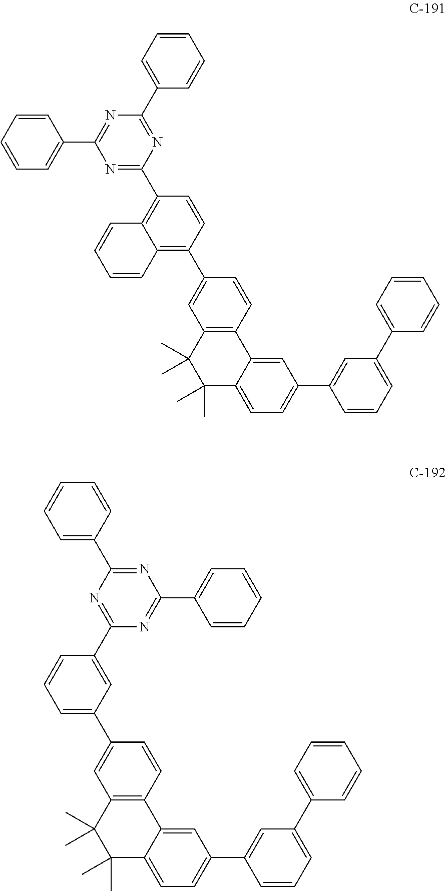 [SMILES: CC1(C)c2ccc(-c3cccc(-c4ccccc4)c3)cc2-c2ccc(-c3ccc(-c4nc(-c5ccccc5)nc(-c5ccccc5)n4)c4ccccc34)cc2C1(C)C ; CC1(C)c2ccc(-c3cccc(-c4ccccc4)c3)cc2-c2ccc(-c3cccc(-c4nc(-c5ccccc5)nc(-c5ccccc5)n4)c3)cc2C1(C)C]

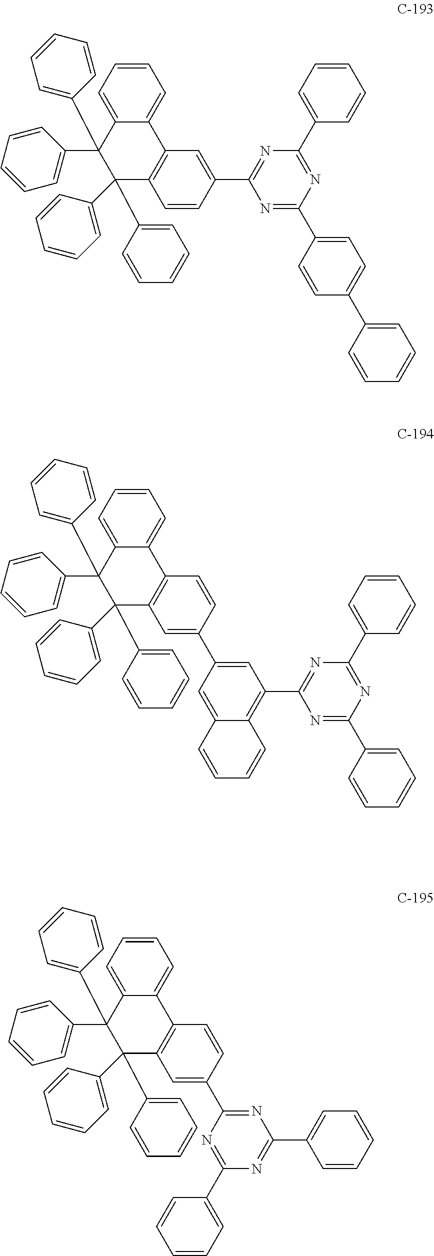 [SMILES: c1ccc(-c2ccc(-c3nc(-c4ccccc4)nc(-c4ccc5c(c4)-c4ccccc4C(c4ccccc4)(c4ccccc4)C5(c4ccccc4)c4ccccc4)n3)cc2)cc1 ; c1ccc(-c2nc(-c3ccccc3)nc(-c3cc(-c4ccc5c(c4)C(c4ccccc4)(c4ccccc4)C(c4ccccc4)(c4ccccc4)c4ccccc4-5)cc4ccccc34)n2)cc1 ; c1ccc(-c2nc(-c3ccccc3)nc(-c3ccc4c(c3)C(c3ccccc3)(c3ccccc3)C(c3ccccc3)(c3ccccc3)c3ccccc3-4)n2)cc1]

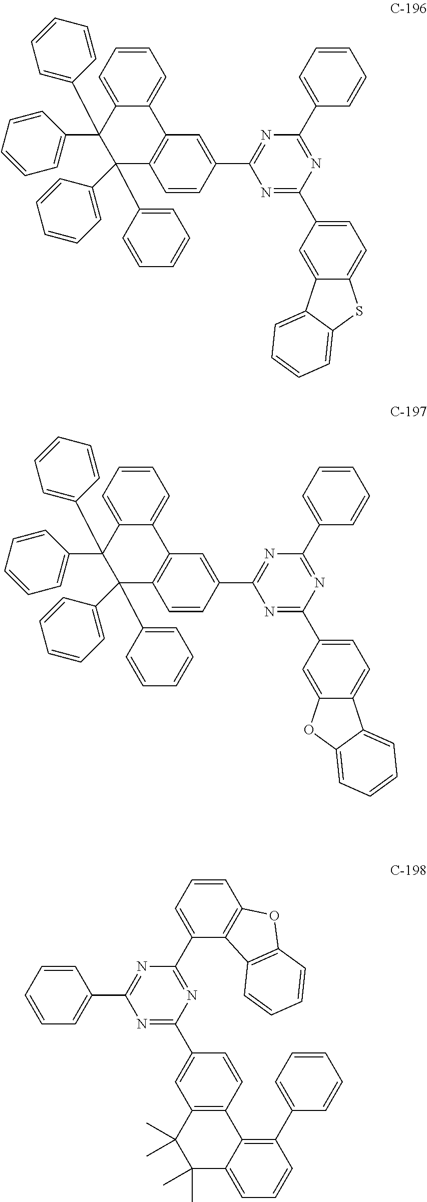 [SMILES: c1ccc(-c2nc(-c3ccc4c(c3)-c3ccccc3C(c3ccccc3)(c3ccccc3)C4(c3ccccc3)c3ccccc3)nc(-c3ccc4sc5ccccc5c4c3)n2)cc1 ; c1ccc(-c2nc(-c3ccc4c(c3)-c3ccccc3C(c3ccccc3)(c3ccccc3)C4(c3ccccc3)c3ccccc3)nc(-c3ccc4c(c3)oc3ccccc34)n2)cc1 ; CC1(C)c2cc(-c3nc(-c4ccccc4)nc(-c4cccc5oc6ccccc6c45)n3)ccc2-c2c(-c3ccccc3)cccc2C1(C)C]

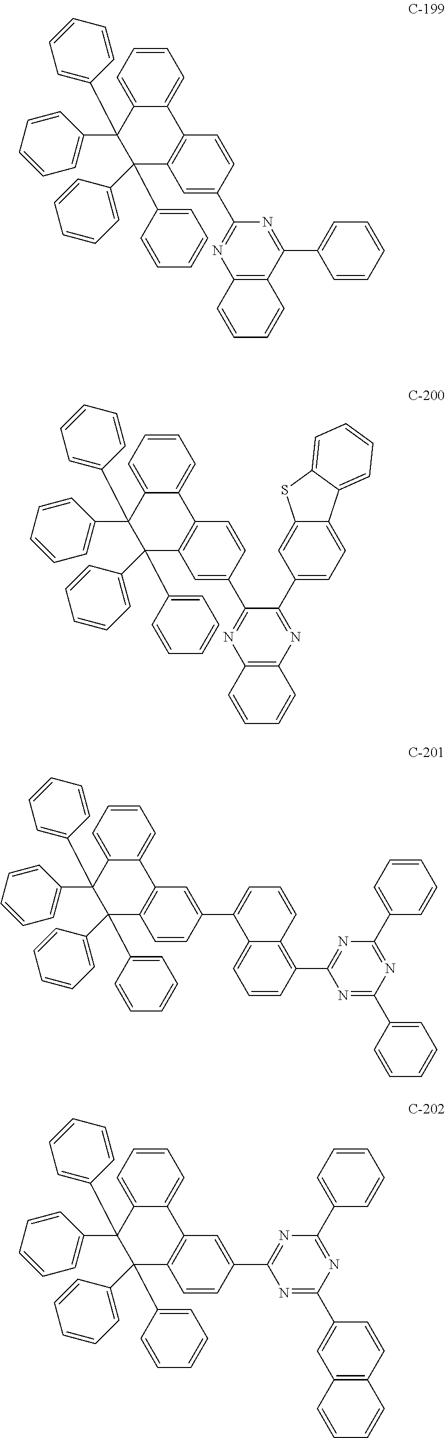 [SMILES: c1ccc(-c2nc(-c3ccc4c(c3)C(c3ccccc3)(c3ccccc3)C(c3ccccc3)(c3ccccc3)c3ccccc3-4)nc3ccccc23)cc1 ; c1ccc(C2(c3ccccc3)c3ccccc3-c3ccc(-c4nc5ccccc5nc4-c4ccc5c(c4)sc4ccccc45)cc3C2(c2ccccc2)c2ccccc2)cc1 ; c1ccc(-c2nc(-c3ccccc3)nc(-c3cccc4c(-c5ccc6c(c5)-c5ccccc5C(c5ccccc5)(c5ccccc5)C6(c5ccccc5)c5ccccc5)cccc34)n2)cc1 ; c1ccc(-c2nc(-c3ccc4c(c3)-c3ccccc3C(c3ccccc3)(c3ccccc3)C4(c3ccccc3)c3ccccc3)nc(-c3ccc4ccccc4c3)n2)cc1]

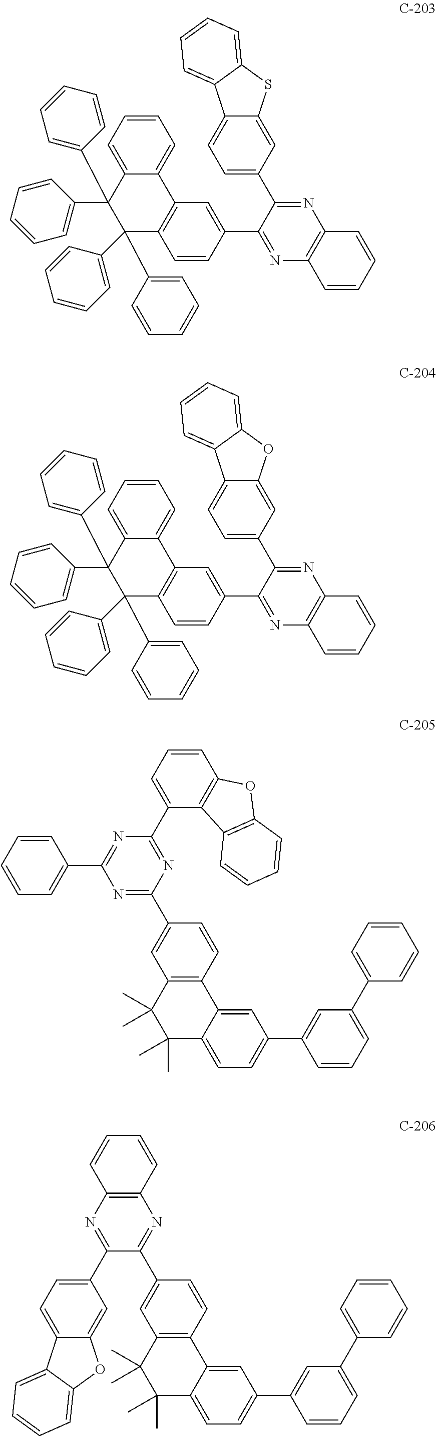 [SMILES: c1ccc(C2(c3ccccc3)c3ccccc3-c3cc(-c4nc5ccccc5nc4-c4ccc5c(c4)sc4ccccc45)ccc3C2(c2ccccc2)c2ccccc2)cc1 ; c1ccc(C2(c3ccccc3)c3ccccc3-c3cc(-c4nc5ccccc5nc4-c4ccc5c(c4)oc4ccccc45)ccc3C2(c2ccccc2)c2ccccc2)cc1 ; CC1(C)c2ccc(-c3cccc(-c4ccccc4)c3)cc2-c2ccc(-c3nc(-c4ccccc4)nc(-c4cccc5oc6ccccc6c45)n3)cc2C1(C)C ; CC1(C)c2ccc(-c3cccc(-c4ccccc4)c3)cc2-c2ccc(-c3nc4ccccc4nc3-c3ccc4c(c3)oc3ccccc34)cc2C1(C)C]

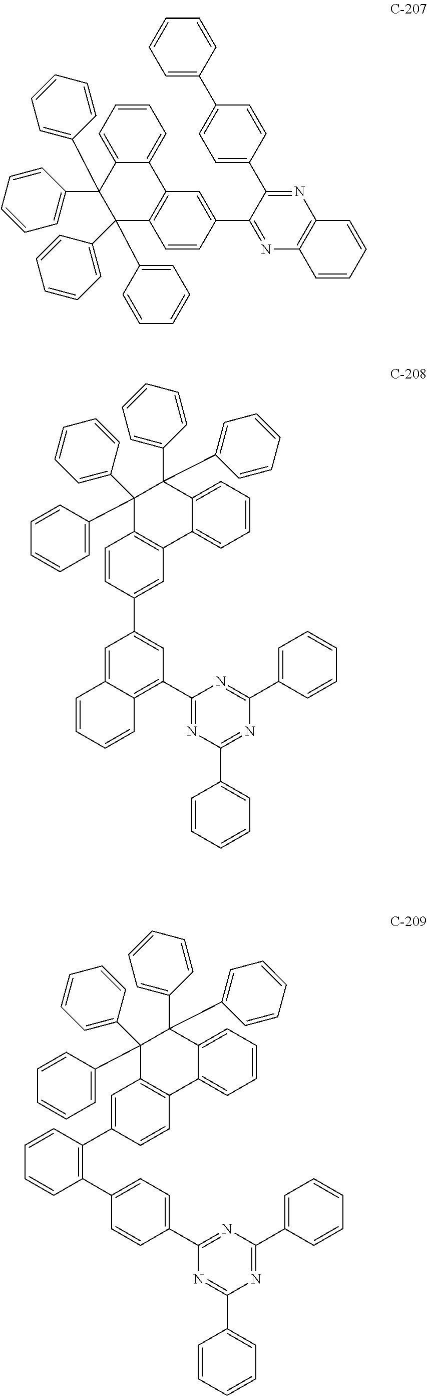 [SMILES: c1ccc(-c2ccc(-c3nc4ccccc4nc3-c3ccc4c(c3)-c3ccccc3C(c3ccccc3)(c3ccccc3)C4(c3ccccc3)c3ccccc3)cc2)cc1 ; c1ccc(-c2nc(-c3ccccc3)nc(-c3cc(-c4ccc5c(c4)-c4ccccc4C(c4ccccc4)(c4ccccc4)C5(c4ccccc4)c4ccccc4)cc4ccccc34)n2)cc1 ; c1ccc(-c2nc(-c3ccccc3)nc(-c3ccc(-c4ccccc4-c4ccc5c(c4)C(c4ccccc4)(c4ccccc4)C(c4ccccc4)(c4ccccc4)c4ccccc4-5)cc3)n2)cc1]

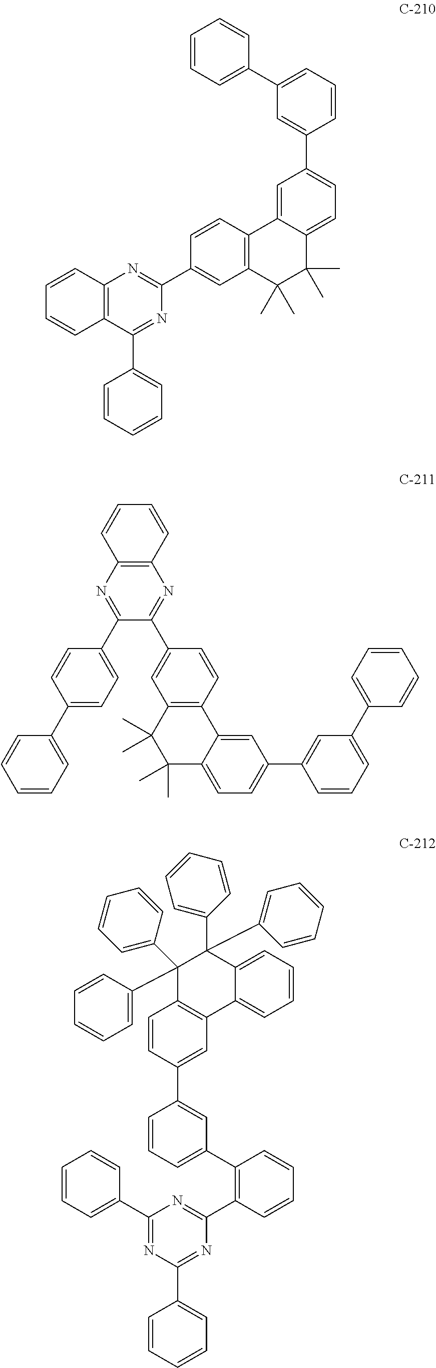 [SMILES: CC1(C)c2ccc(-c3cccc(-c4ccccc4)c3)cc2-c2ccc(-c3nc(-c4ccccc4)c4ccccc4n3)cc2C1(C)C ; CC1(C)c2ccc(-c3cccc(-c4ccccc4)c3)cc2-c2ccc(-c3nc4ccccc4nc3-c3ccc(-c4ccccc4)cc3)cc2C1(C)C ; c1ccc(-c2nc(-c3ccccc3)nc(-c3ccccc3-c3cccc(-c4ccc5c(c4)-c4ccccc4C(c4ccccc4)(c4ccccc4)C5(c4ccccc4)c4ccccc4)c3)n2)cc1]

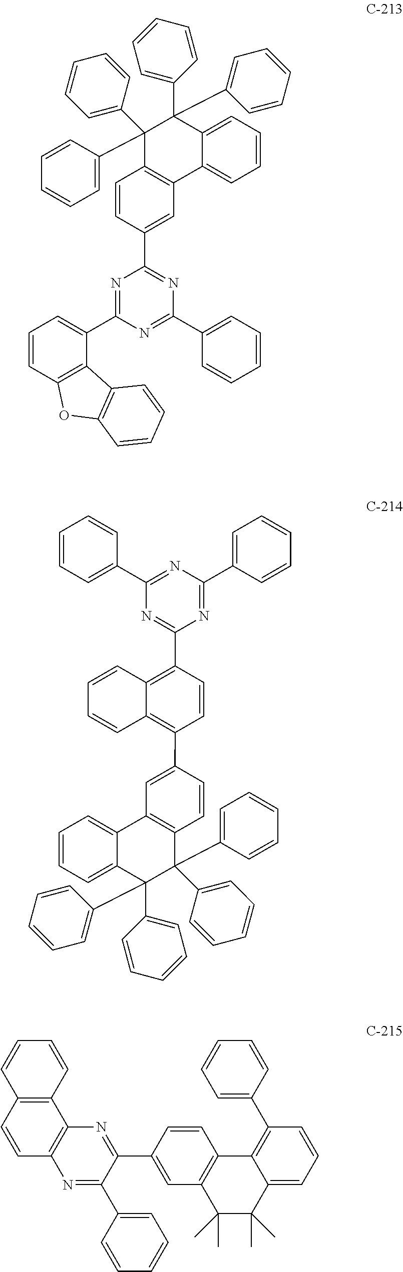 [SMILES: c1ccc(-c2nc(-c3ccc4c(c3)-c3ccccc3C(c3ccccc3)(c3ccccc3)C4(c3ccccc3)c3ccccc3)nc(-c3cccc4oc5ccccc5c34)n2)cc1 ; c1ccc(-c2nc(-c3ccccc3)nc(-c3ccc(-c4ccc5c(c4)-c4ccccc4C(c4ccccc4)(c4ccccc4)C5(c4ccccc4)c4ccccc4)c4ccccc34)n2)cc1 ; CC1(C)c2cc(-c3nc4c(ccc5ccccc54)nc3-c3ccccc3)ccc2-c2c(-c3ccccc3)cccc2C1(C)C]

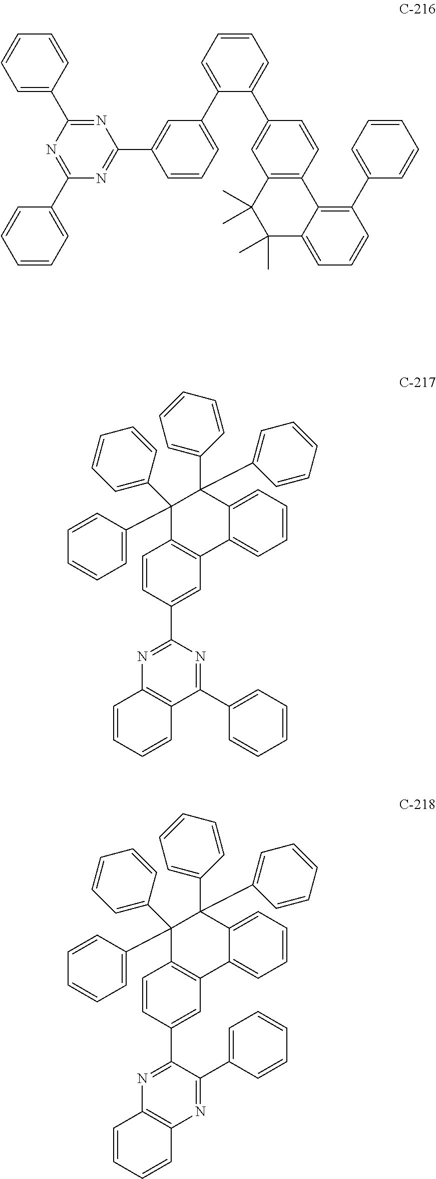 [SMILES: CC1(C)c2cc(-c3ccccc3-c3cccc(-c4nc(-c5ccccc5)nc(-c5ccccc5)n4)c3)ccc2-c2c(-c3ccccc3)cccc2C1(C)C ; c1ccc(-c2nc(-c3ccc4c(c3)-c3ccccc3C(c3ccccc3)(c3ccccc3)C4(c3ccccc3)c3ccccc3)nc3ccccc23)cc1 ; c1ccc(-c2nc3ccccc3nc2-c2ccc3c(c2)-c2ccccc2C(c2ccccc2)(c2ccccc2)C3(c2ccccc2)c2ccccc2)cc1]

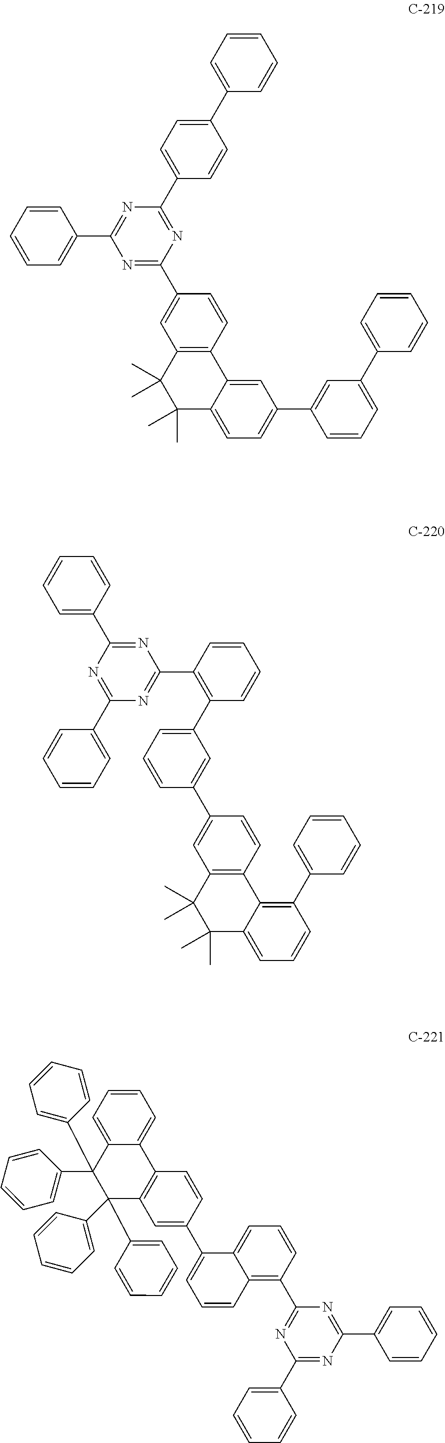 [SMILES: CC1(C)c2ccc(-c3cccc(-c4ccccc4)c3)cc2-c2ccc(-c3nc(-c4ccccc4)nc(-c4ccc(-c5ccccc5)cc4)n3)cc2C1(C)C ; CC1(C)c2cc(-c3cccc(-c4ccccc4-c4nc(-c5ccccc5)nc(-c5ccccc5)n4)c3)ccc2-c2c(-c3ccccc3)cccc2C1(C)C ; c1ccc(-c2nc(-c3ccccc3)nc(-c3cccc4c(-c5ccc6c(c5)C(c5ccccc5)(c5ccccc5)C(c5ccccc5)(c5ccccc5)c5ccccc5-6)cccc34)n2)cc1]

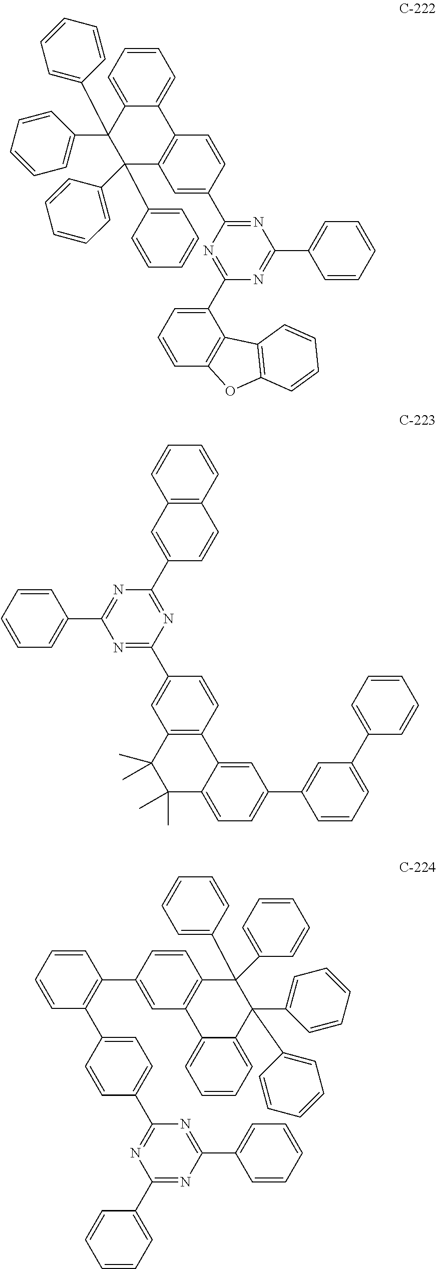 [SMILES: c1ccc(-c2nc(-c3ccc4c(c3)C(c3ccccc3)(c3ccccc3)C(c3ccccc3)(c3ccccc3)c3ccccc3-4)nc(-c3cccc4oc5ccccc5c34)n2)cc1 ; CC1(C)c2ccc(-c3cccc(-c4ccccc4)c3)cc2-c2ccc(-c3nc(-c4ccccc4)nc(-c4ccc5ccccc5c4)n3)cc2C1(C)C ; c1ccc(-c2nc(-c3ccccc3)nc(-c3ccc(-c4ccccc4-c4ccc5c(c4)-c4ccccc4C(c4ccccc4)(c4ccccc4)C5(c4ccccc4)c4ccccc4)cc3)n2)cc1]

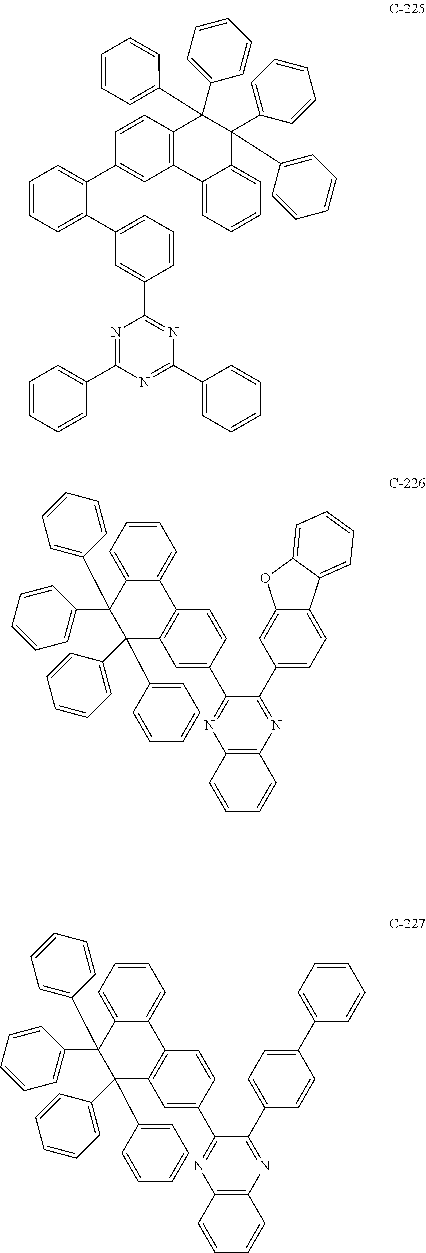 [SMILES: c1ccc(-c2nc(-c3ccccc3)nc(-c3cccc(-c4ccccc4-c4ccc5c(c4)-c4ccccc4C(c4ccccc4)(c4ccccc4)C5(c4ccccc4)c4ccccc4)c3)n2)cc1 ; c1ccc(C2(c3ccccc3)c3ccccc3-c3ccc(-c4nc5ccccc5nc4-c4ccc5c(c4)oc4ccccc45)cc3C2(c2ccccc2)c2ccccc2)cc1 ; c1ccc(-c2ccc(-c3nc4ccccc4nc3-c3ccc4c(c3)C(c3ccccc3)(c3ccccc3)C(c3ccccc3)(c3ccccc3)c3ccccc3-4)cc2)cc1]

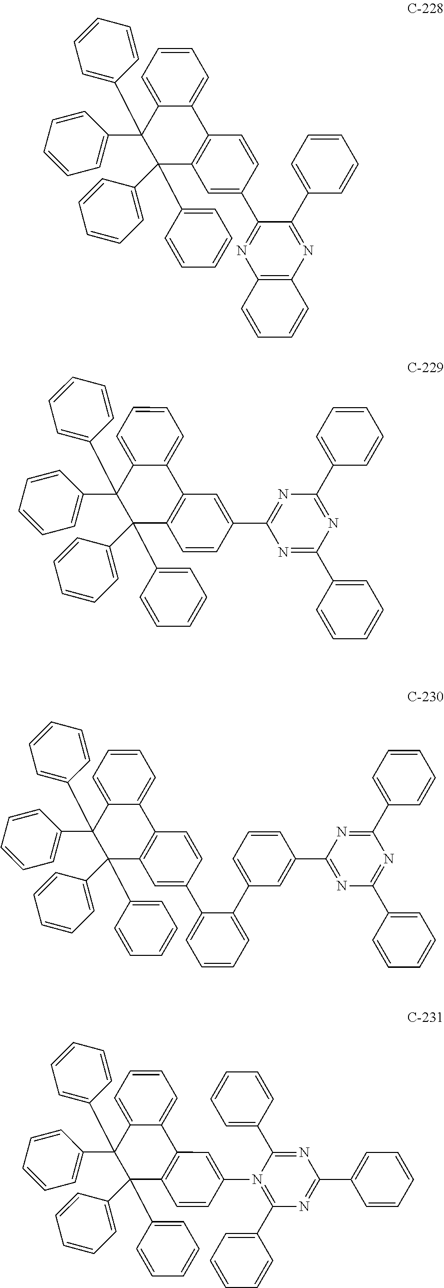

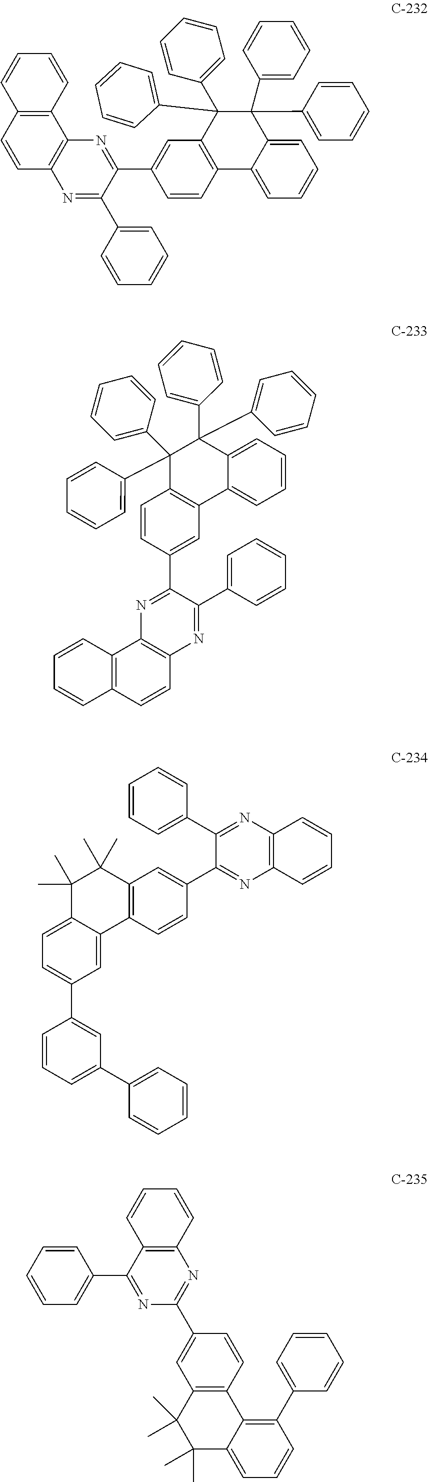 [SMILES: c1ccc(-c2nc3ccc4ccccc4c3nc2-c2ccc3c(c2)C(c2ccccc2)(c2ccccc2)C(c2ccccc2)(c2ccccc2)c2ccccc2-3)cc1 ; c1ccc(-c2nc3ccc4ccccc4c3nc2-c2ccc3c(c2)-c2ccccc2C(c2ccccc2)(c2ccccc2)C3(c2ccccc2)c2ccccc2)cc1 ; CC1(C)c2ccc(-c3cccc(-c4ccccc4)c3)cc2-c2ccc(-c3nc4ccccc4nc3-c3ccccc3)cc2C1(C)C ; CC1(C)c2cc(-c3nc(-c4ccccc4)c4ccccc4n3)ccc2-c2c(-c3ccccc3)cccc2C1(C)C]

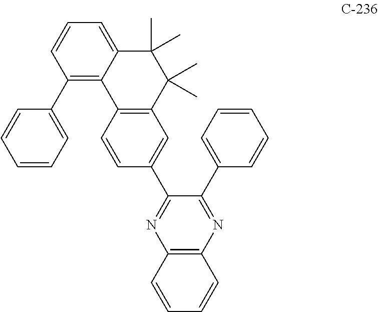 [SMILES: CC1(C)c2cc(-c3nc4ccccc4nc3-c3ccccc3)ccc2-c2c(-c3ccccc3)cccc2C1(C)C]

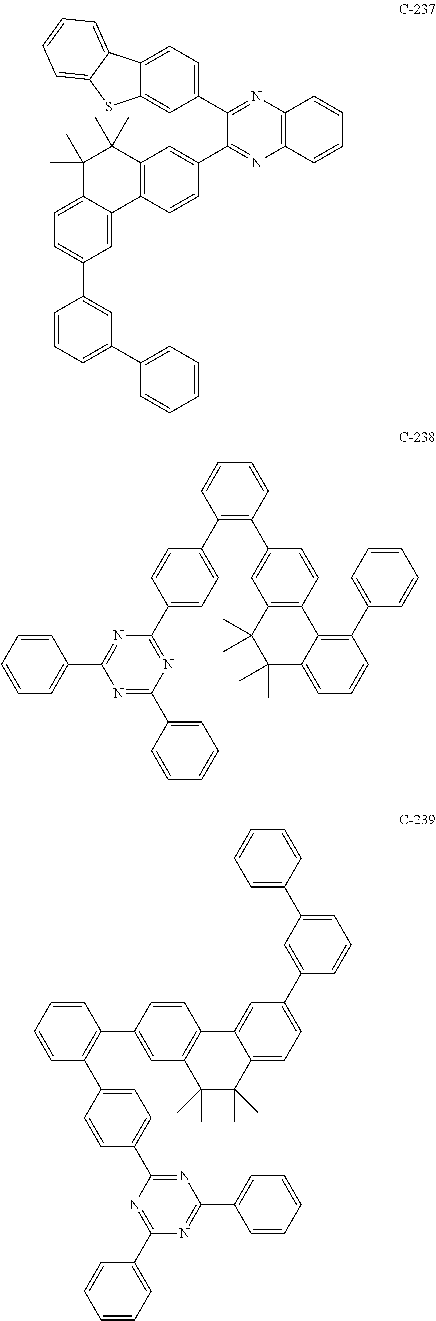 [SMILES: CC1(C)c2ccc(-c3cccc(-c4ccccc4)c3)cc2-c2ccc(-c3nc4ccccc4nc3-c3ccc4c(c3)sc3ccccc34)cc2C1(C)C ; CC1(C)c2cc(-c3ccccc3-c3ccc(-c4nc(-c5ccccc5)nc(-c5ccccc5)n4)cc3)ccc2-c2c(-c3ccccc3)cccc2C1(C)C ; CC1(C)c2ccc(-c3cccc(-c4ccccc4)c3)cc2-c2ccc(-c3ccccc3-c3ccc(-c4nc(-c5ccccc5)nc(-c5ccccc5)n4)cc3)cc2C1(C)C]

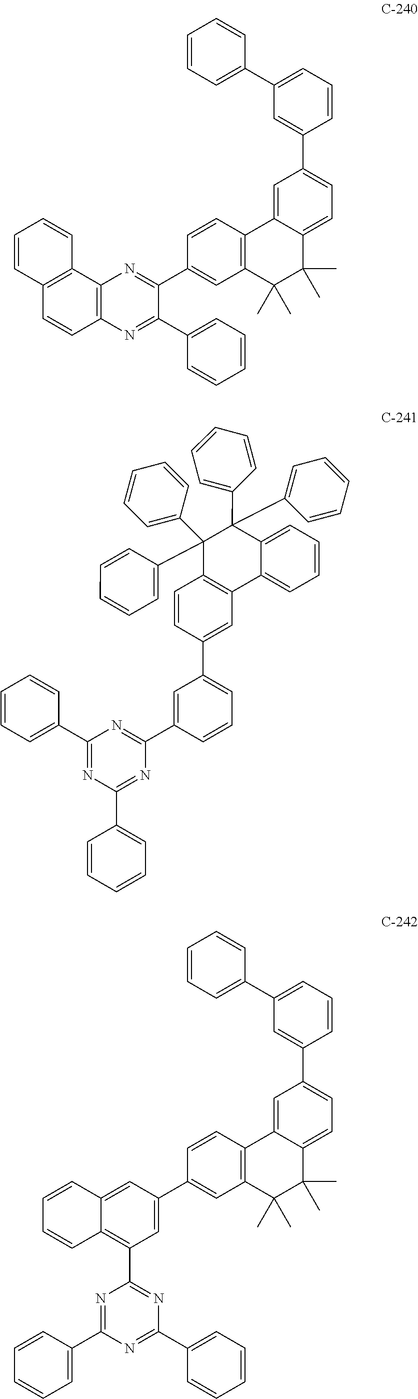

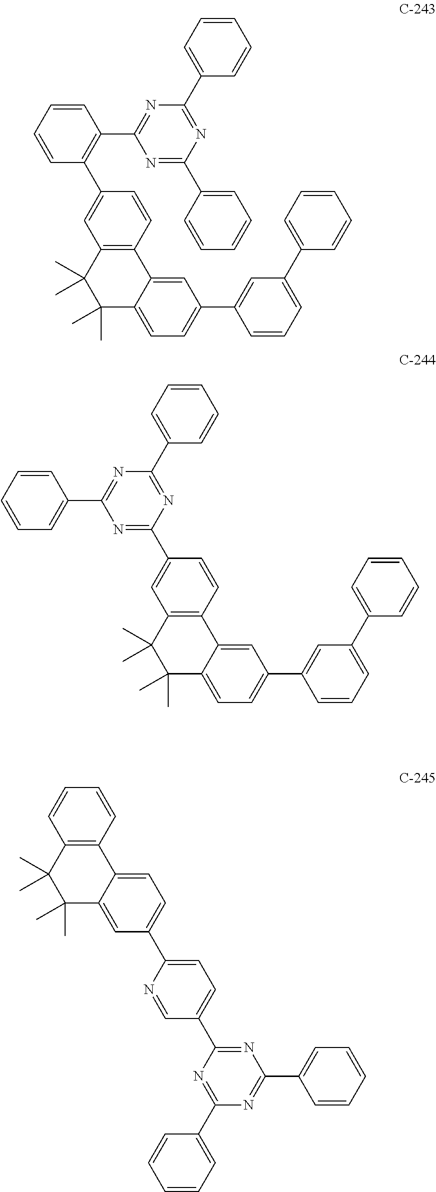 [SMILES: CC1(C)c2ccc(-c3cccc(-c4ccccc4)c3)cc2-c2ccc(-c3ccccc3-c3nc(-c4ccccc4)nc(-c4ccccc4)n3)cc2C1(C)C ; CC1(C)c2ccc(-c3cccc(-c4ccccc4)c3)cc2-c2ccc(-c3nc(-c4ccccc4)nc(-c4ccccc4)n3)cc2C1(C)C ; CC1(C)c2ccccc2-c2ccc(-c3ccc(-c4nc(-c5ccccc5)nc(-c5ccccc5)n4)cn3)cc2C1(C)C]

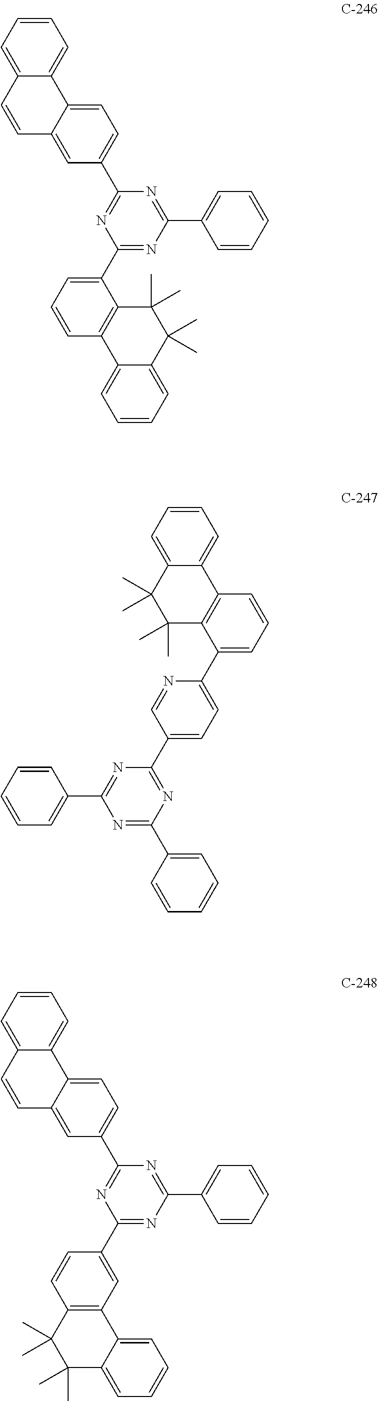 [SMILES: CC1(C)c2ccccc2-c2cccc(-c3nc(-c4ccccc4)nc(-c4ccc5c(ccc6ccccc65)c4)n3)c2C1(C)C ; CC1(C)c2ccccc2-c2cccc(-c3ccc(-c4nc(-c5ccccc5)nc(-c5ccccc5)n4)cn3)c2C1(C)C ; CC1(C)c2ccccc2-c2cc(-c3nc(-c4ccccc4)nc(-c4ccc5c(ccc6ccccc65)c4)n3)ccc2C1(C)C]

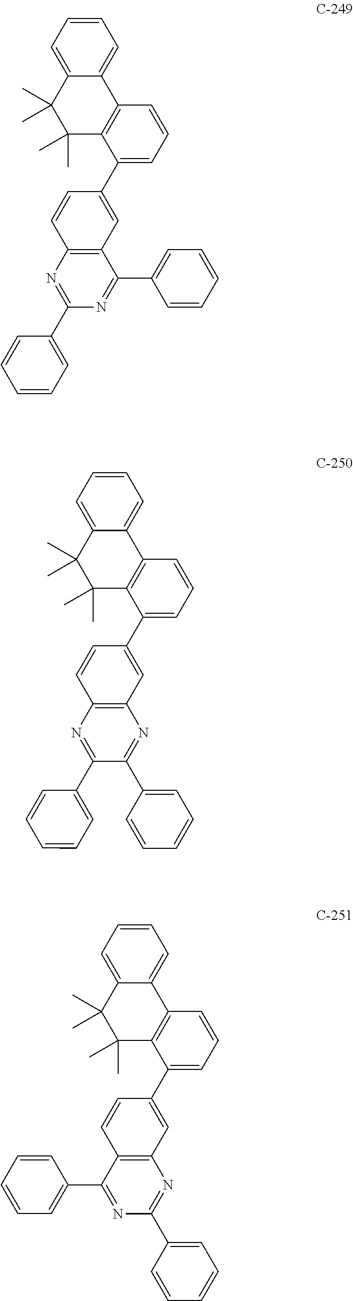 [SMILES: CC1(C)c2ccccc2-c2cccc(-c3ccc4nc(-c5ccccc5)nc(-c5ccccc5)c4c3)c2C1(C)C ; CC1(C)c2ccccc2-c2cccc(-c3ccc4nc(-c5ccccc5)c(-c5ccccc5)nc4c3)c2C1(C)C ; CC1(C)c2ccccc2-c2cccc(-c3ccc4c(-c5ccccc5)nc(-c5ccccc5)nc4c3)c2C1(C)C]

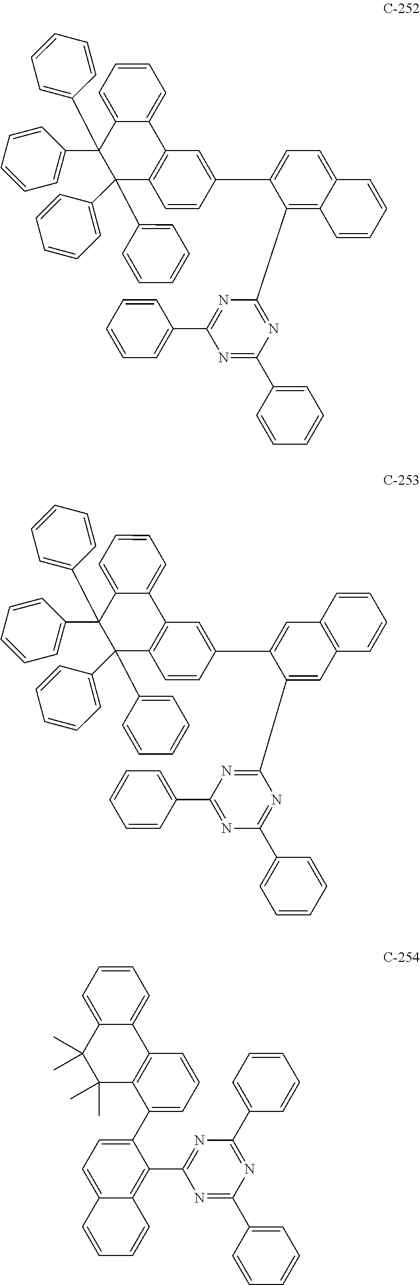 [SMILES: c1ccc(-c2nc(-c3ccccc3)nc(-c3c(-c4ccc5c(c4)-c4ccccc4C(c4ccccc4)(c4ccccc4)C5(c4ccccc4)c4ccccc4)ccc4ccccc34)n2)cc1 ; c1ccc(-c2nc(-c3ccccc3)nc(-c3cc4ccccc4cc3-c3ccc4c(c3)-c3ccccc3C(c3ccccc3)(c3ccccc3)C4(c3ccccc3)c3ccccc3)n2)cc1 ; CC1(C)c2ccccc2-c2cccc(-c3ccc4ccccc4c3-c3nc(-c4ccccc4)nc(-c4ccccc4)n3)c2C1(C)C]

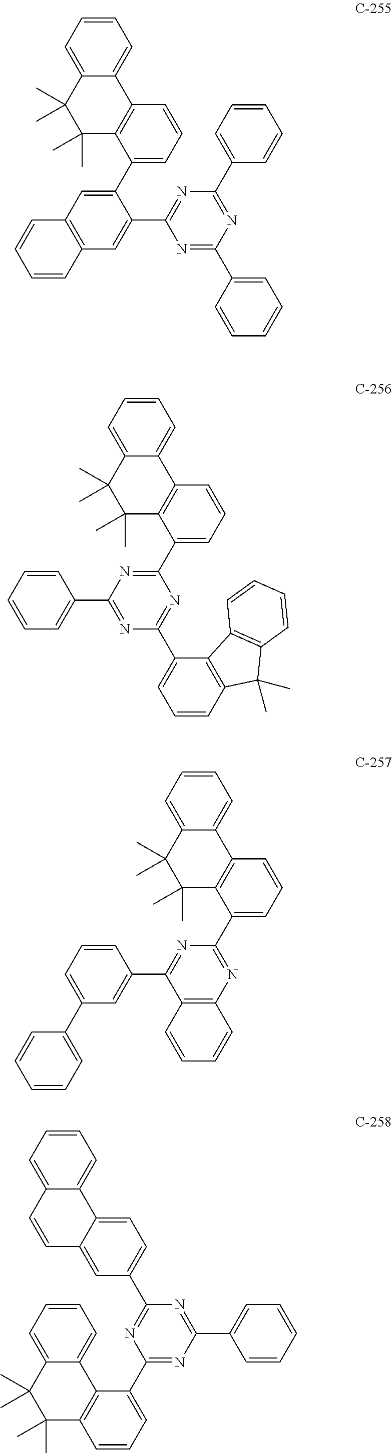

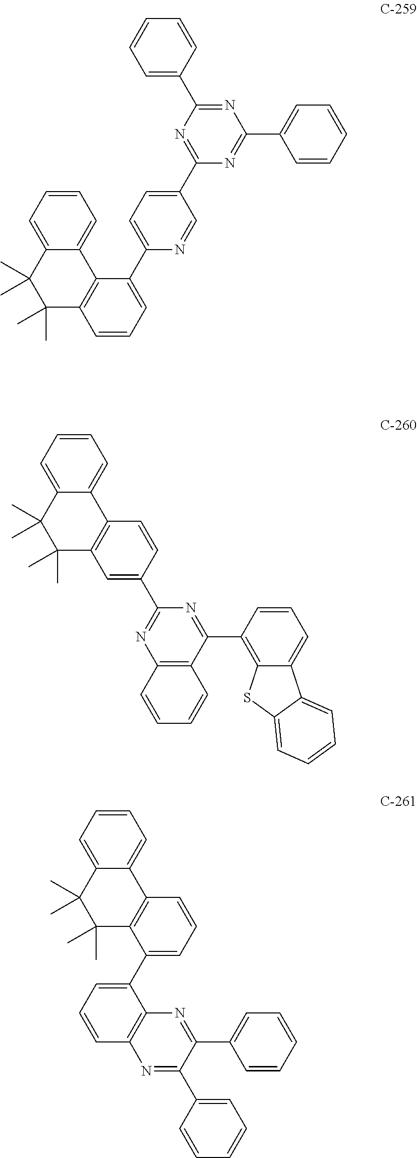 [SMILES: CC1(C)c2ccccc2-c2c(-c3ccc(-c4nc(-c5ccccc5)nc(-c5ccccc5)n4)cn3)cccc2C1(C)C ; CC1(C)c2ccccc2-c2ccc(-c3nc(-c4cccc5c4sc4ccccc45)c4ccccc4n3)cc2C1(C)C ; CC1(C)c2ccccc2-c2cccc(-c3cccc4nc(-c5ccccc5)c(-c5ccccc5)nc34)c2C1(C)C]

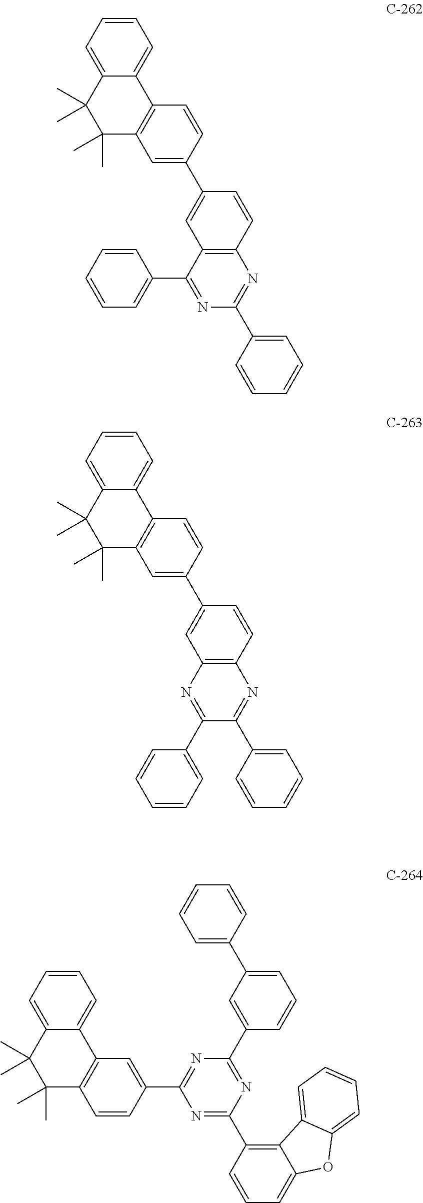 [SMILES: CC1(C)c2ccccc2-c2ccc(-c3ccc4nc(-c5ccccc5)nc(-c5ccccc5)c4c3)cc2C1(C)C ; CC1(C)c2ccccc2-c2ccc(-c3ccc4nc(-c5ccccc5)c(-c5ccccc5)nc4c3)cc2C1(C)C ; CC1(C)c2ccccc2-c2cc(-c3nc(-c4cccc(-c5ccccc5)c4)nc(-c4cccc5oc6ccccc6c45)n3)ccc2C1(C)C]

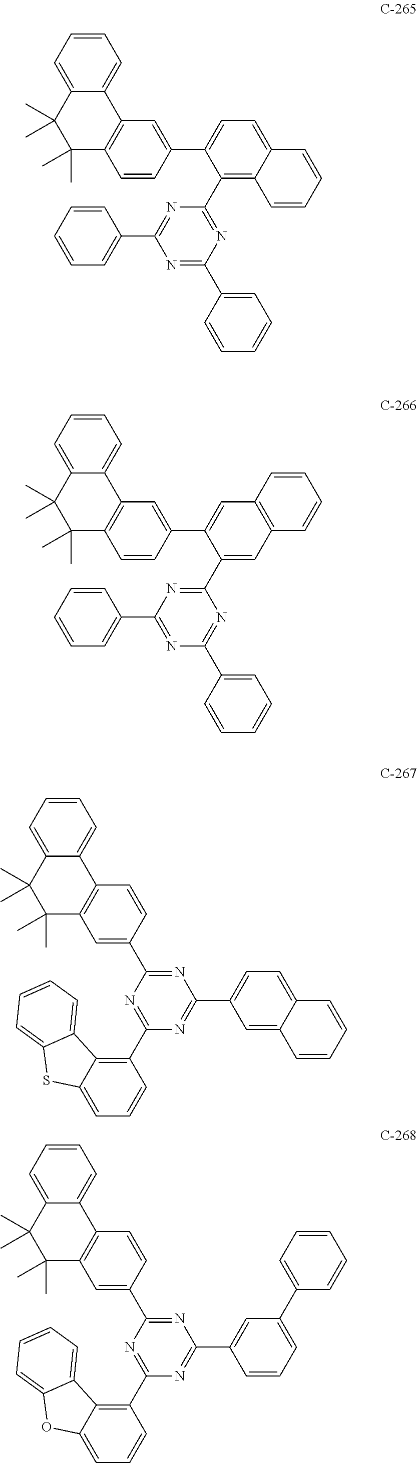

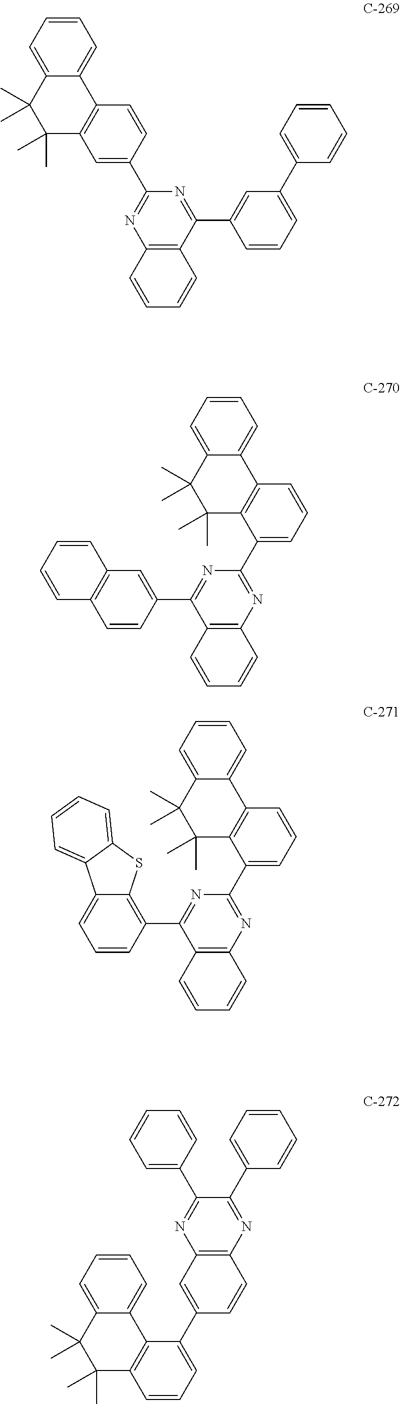

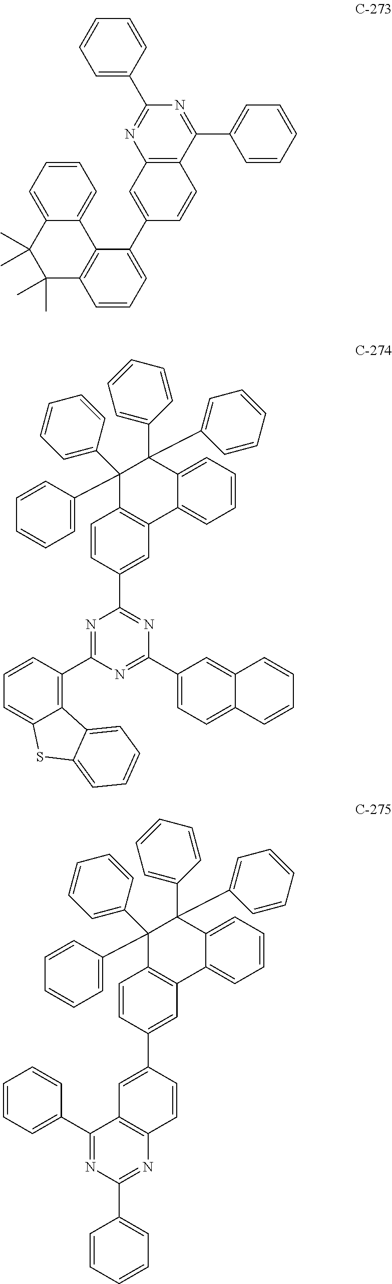 [SMILES: CC1(C)c2ccccc2-c2c(-c3ccc4c(-c5ccccc5)nc(-c5ccccc5)nc4c3)cccc2C1(C)C ; c1ccc(C2(c3ccccc3)c3ccccc3-c3cc(-c4nc(-c5ccc6ccccc6c5)nc(-c5cccc6sc7ccccc7c56)n4)ccc3C2(c2ccccc2)c2ccccc2)cc1 ; c1ccc(-c2nc(-c3ccccc3)c3cc(-c4ccc5c(c4)-c4ccccc4C(c4ccccc4)(c4ccccc4)C5(c4ccccc4)c4ccccc4)ccc3n2)cc1]

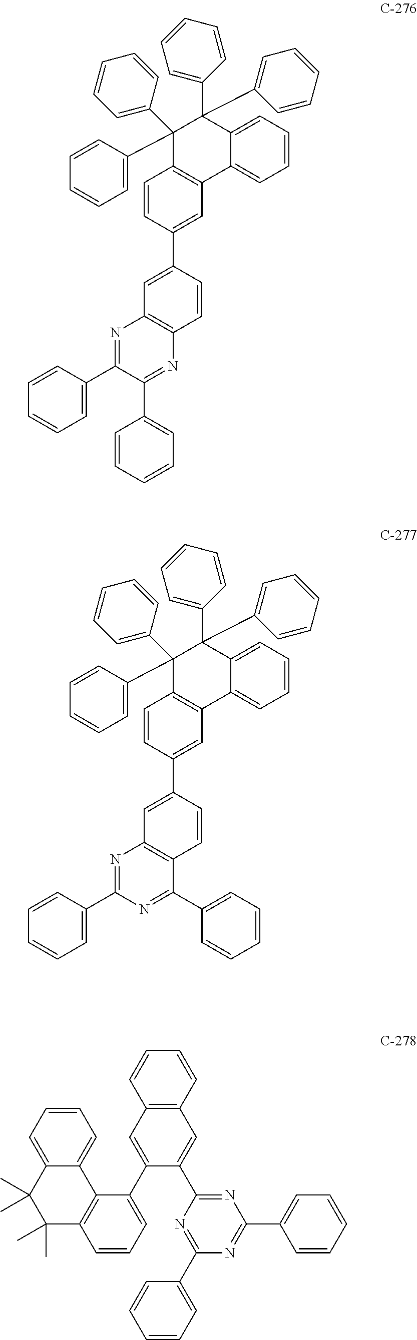 [SMILES: c1ccc(-c2nc3ccc(-c4ccc5c(c4)-c4ccccc4C(c4ccccc4)(c4ccccc4)C5(c4ccccc4)c4ccccc4)cc3nc2-c2ccccc2)cc1 ; c1ccc(-c2nc(-c3ccccc3)c3ccc(-c4ccc5c(c4)-c4ccccc4C(c4ccccc4)(c4ccccc4)C5(c4ccccc4)c4ccccc4)cc3n2)cc1 ; CC1(C)c2ccccc2-c2c(-c3cc4ccccc4cc3-c3nc(-c4ccccc4)nc(-c4ccccc4)n3)cccc2C1(C)C]

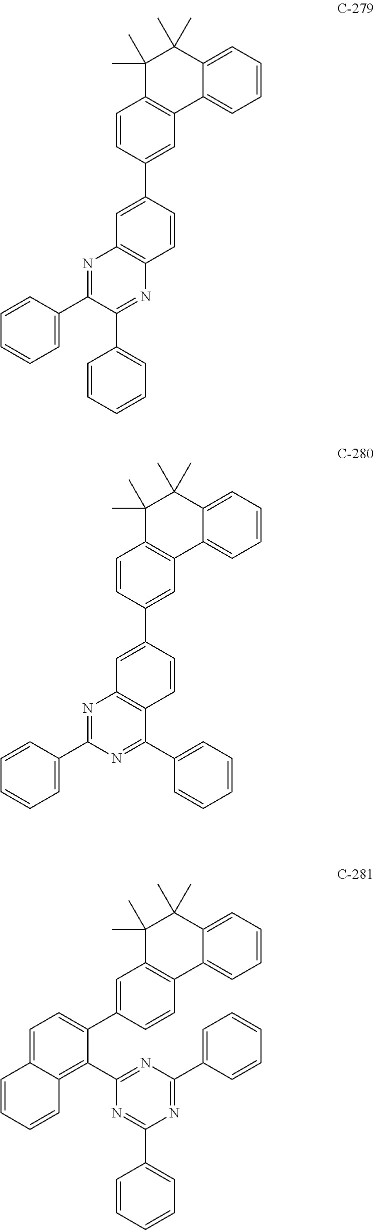 [SMILES: CC1(C)c2ccccc2-c2cc(-c3ccc4nc(-c5ccccc5)c(-c5ccccc5)nc4c3)ccc2C1(C)C ; CC1(C)c2ccccc2-c2cc(-c3ccc4c(-c5ccccc5)nc(-c5ccccc5)nc4c3)ccc2C1(C)C ; CC1(C)c2ccccc2-c2ccc(-c3ccc4ccccc4c3-c3nc(-c4ccccc4)nc(-c4ccccc4)n3)cc2C1(C)C]

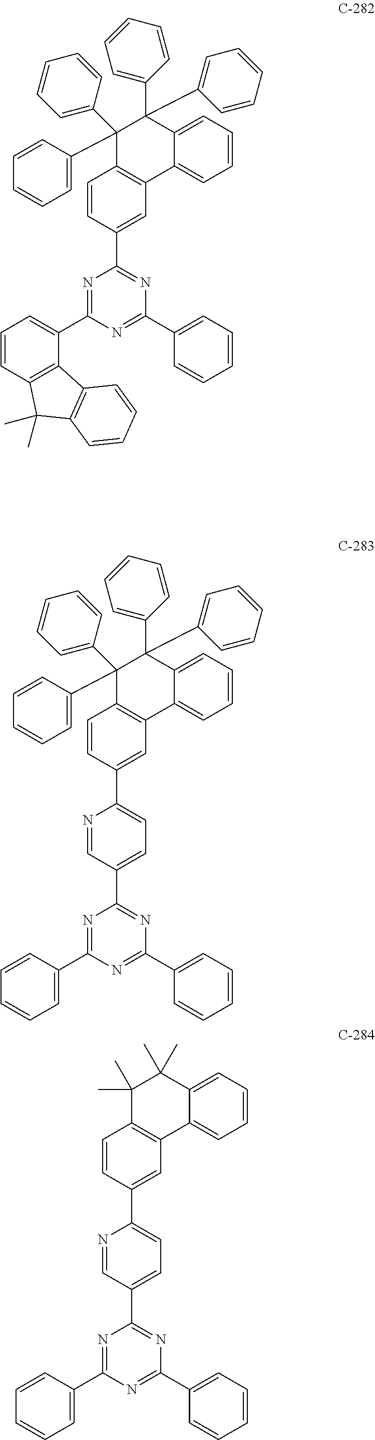

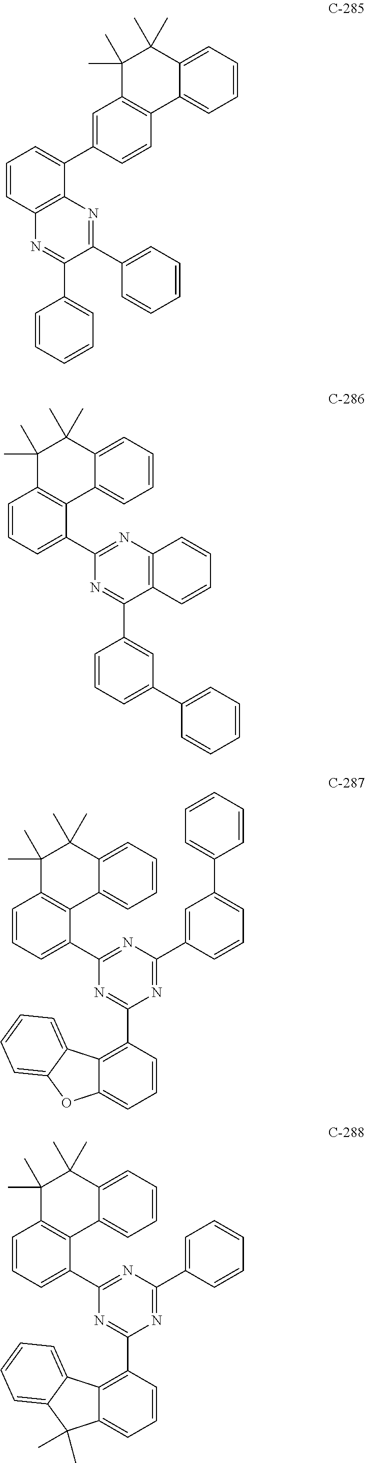

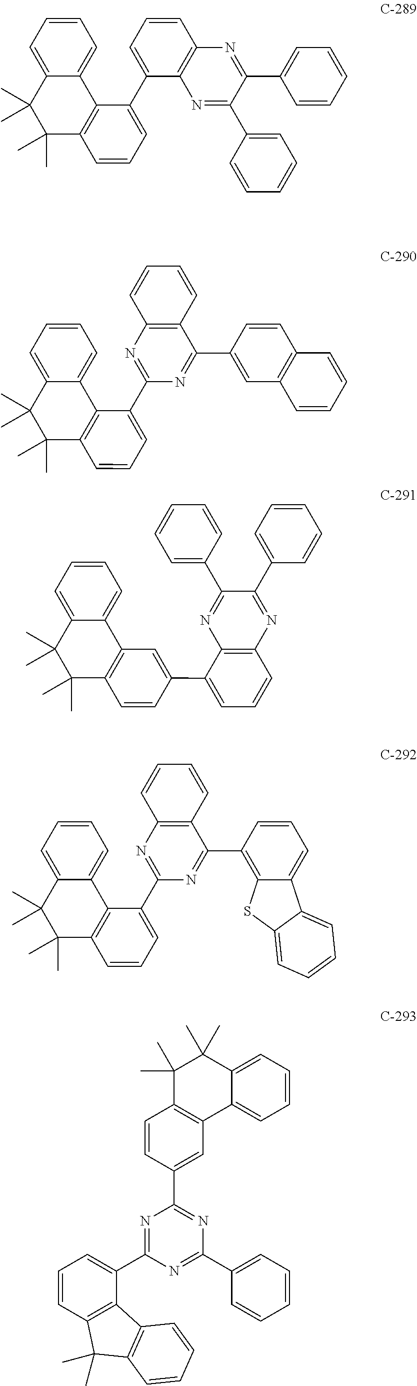 [SMILES: CC1(C)c2ccccc2-c2c(-c3cccc4nc(-c5ccccc5)c(-c5ccccc5)nc34)cccc2C1(C)C ; CC1(C)c2ccccc2-c2c(-c3nc(-c4ccc5ccccc5c4)c4ccccc4n3)cccc2C1(C)C ; CC1(C)c2ccccc2-c2cc(-c3cccc4nc(-c5ccccc5)c(-c5ccccc5)nc34)ccc2C1(C)C ; CC1(C)c2ccccc2-c2c(-c3nc(-c4cccc5c4sc4ccccc45)c4ccccc4n3)cccc2C1(C)C ; CC1(C)c2ccccc2-c2c(-c3nc(-c4ccccc4)nc(-c4ccc5c(c4)-c4ccccc4C(C)(C)C5(C)C)n3)cccc21]

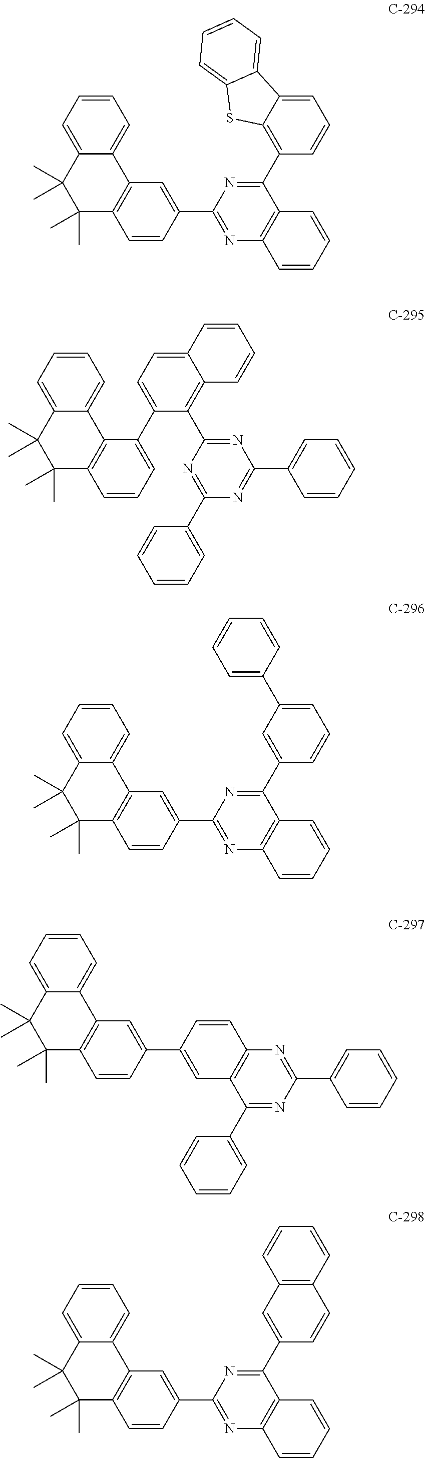 [SMILES: CC1(C)c2ccccc2-c2cc(-c3nc(-c4cccc5c4sc4ccccc45)c4ccccc4n3)ccc2C1(C)C ; CC1(C)c2ccccc2-c2c(-c3ccc4ccccc4c3-c3nc(-c4ccccc4)nc(-c4ccccc4)n3)cccc2C1(C)C ; CC1(C)c2ccccc2-c2cc(-c3nc(-c4cccc(-c5ccccc5)c4)c4ccccc4n3)ccc2C1(C)C ; CC1(C)c2ccccc2-c2cc(-c3ccc4nc(-c5ccccc5)nc(-c5ccccc5)c4c3)ccc2C1(C)C ; CC1(C)c2ccccc2-c2cc(-c3nc(-c4ccc5ccccc5c4)c4ccccc4n3)ccc2C1(C)C]

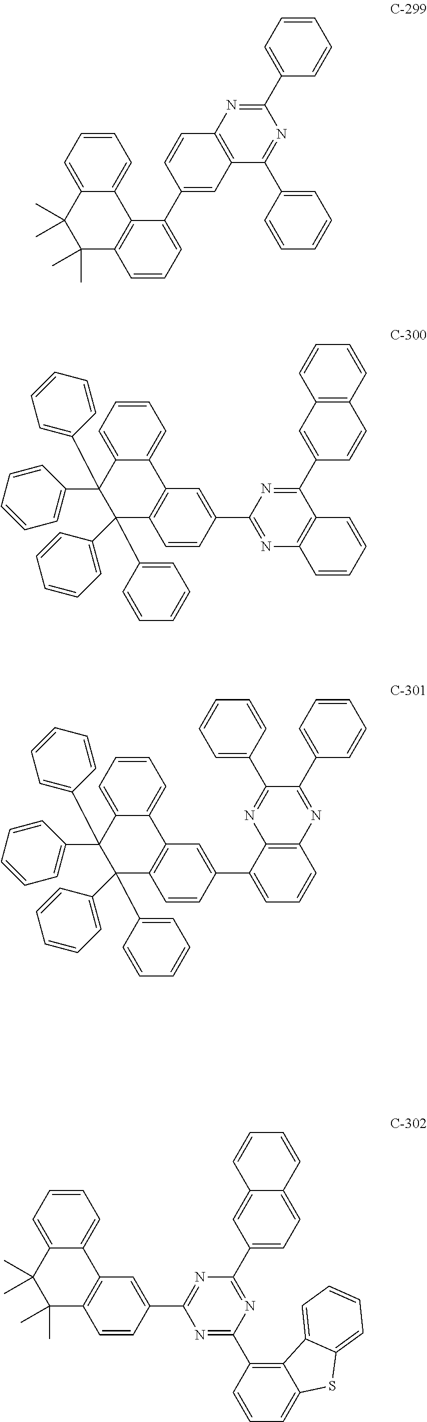 [SMILES: CC1(C)c2ccccc2-c2c(-c3ccc4nc(-c5ccccc5)nc(-c5ccccc5)c4c3)cccc2C1(C)C ; c1ccc(C2(c3ccccc3)c3ccccc3-c3cc(-c4nc(-c5ccc6ccccc6c5)c5ccccc5n4)ccc3C2(c2ccccc2)c2ccccc2)cc1 ; c1ccc(-c2nc3cccc(-c4ccc5c(c4)-c4ccccc4C(c4ccccc4)(c4ccccc4)C5(c4ccccc4)c4ccccc4)c3nc2-c2ccccc2)cc1 ; CC1(C)c2ccccc2-c2cc(-c3nc(-c4ccc5ccccc5c4)nc(-c4cccc5sc6ccccc6c45)n3)ccc2C1(C)C]

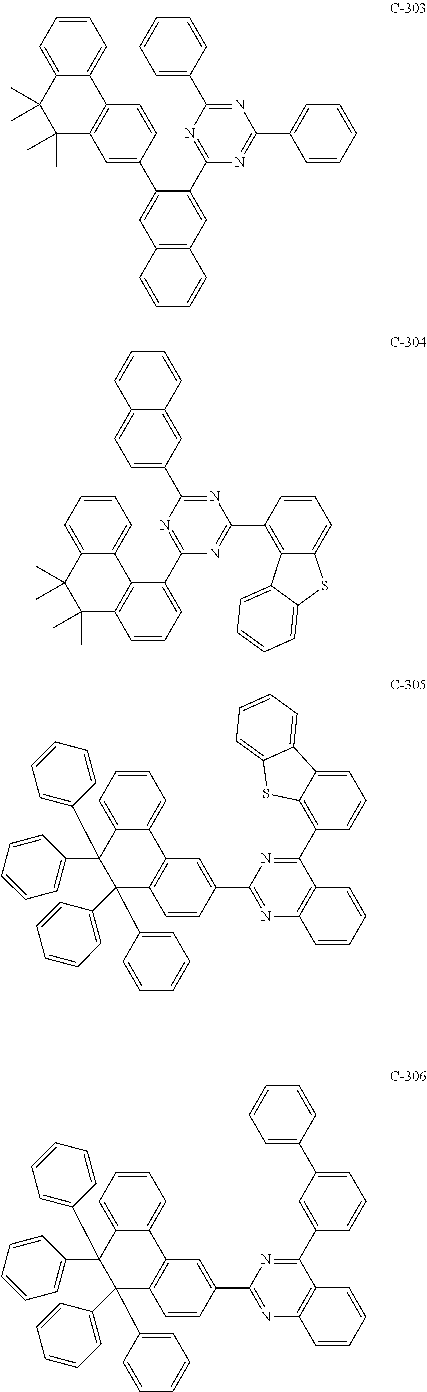

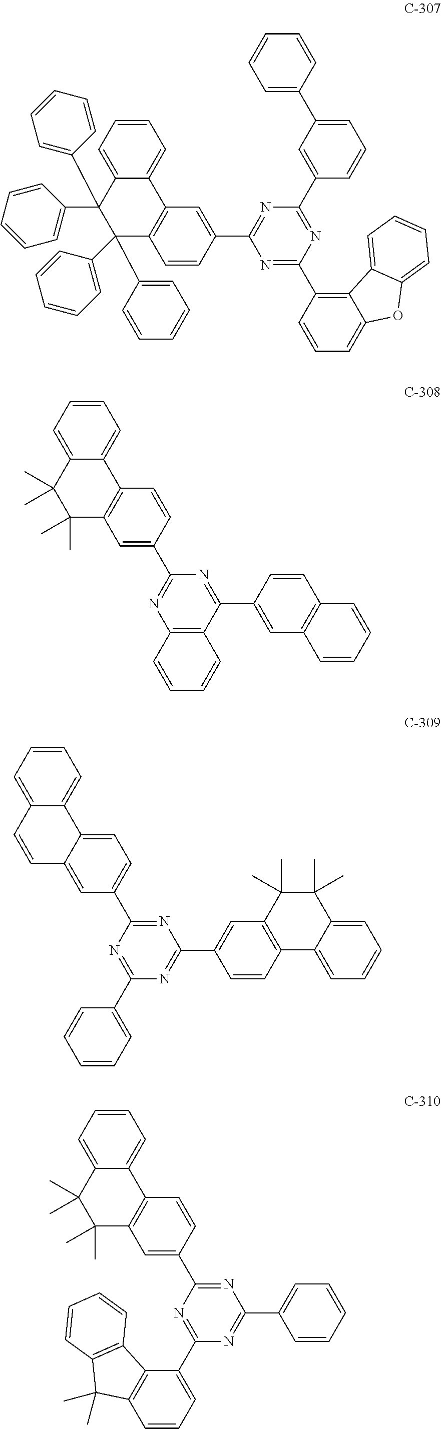 [SMILES: c1ccc(-c2cccc(-c3nc(-c4ccc5c(c4)-c4ccccc4C(c4ccccc4)(c4ccccc4)C5(c4ccccc4)c4ccccc4)nc(-c4cccc5oc6ccccc6c45)n3)c2)cc1 ; CC1(C)c2ccccc2-c2ccc(-c3nc(-c4ccc5ccccc5c4)c4ccccc4n3)cc2C1(C)C ; CC1(C)c2ccccc2-c2ccc(-c3nc(-c4ccccc4)nc(-c4ccc5c(ccc6ccccc65)c4)n3)cc2C1(C)C ; CC1(C)c2ccccc2-c2c(-c3nc(-c4ccccc4)nc(-c4ccc5c(c4)C(C)(C)C(C)(C)c4ccccc4-5)n3)cccc21]

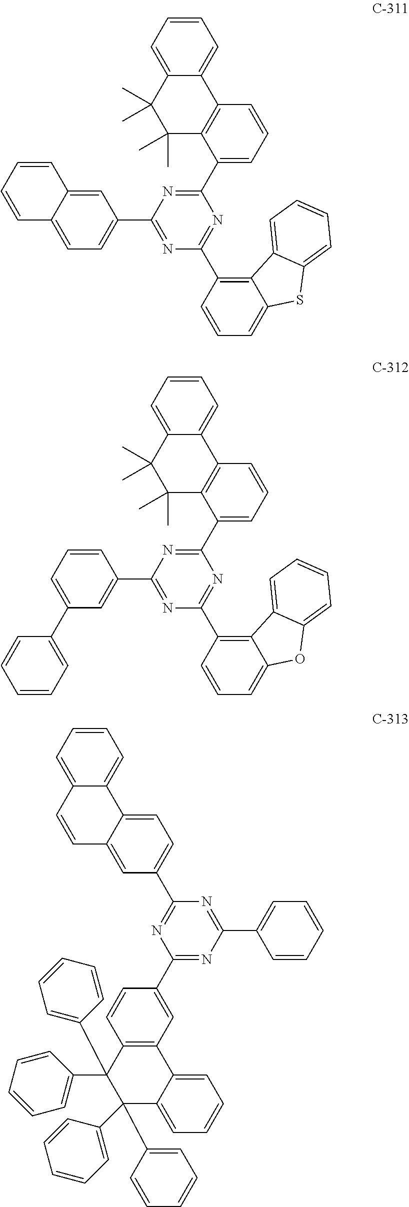

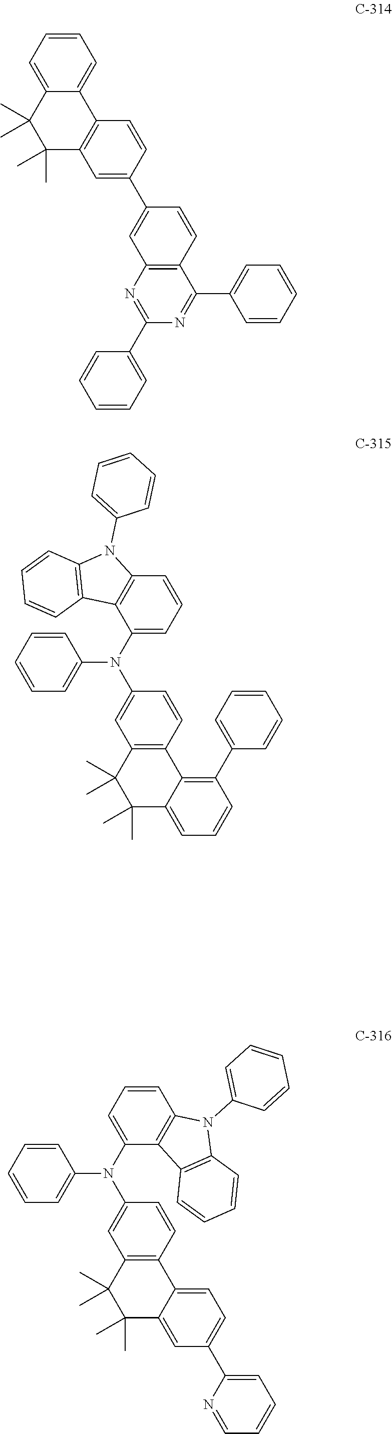

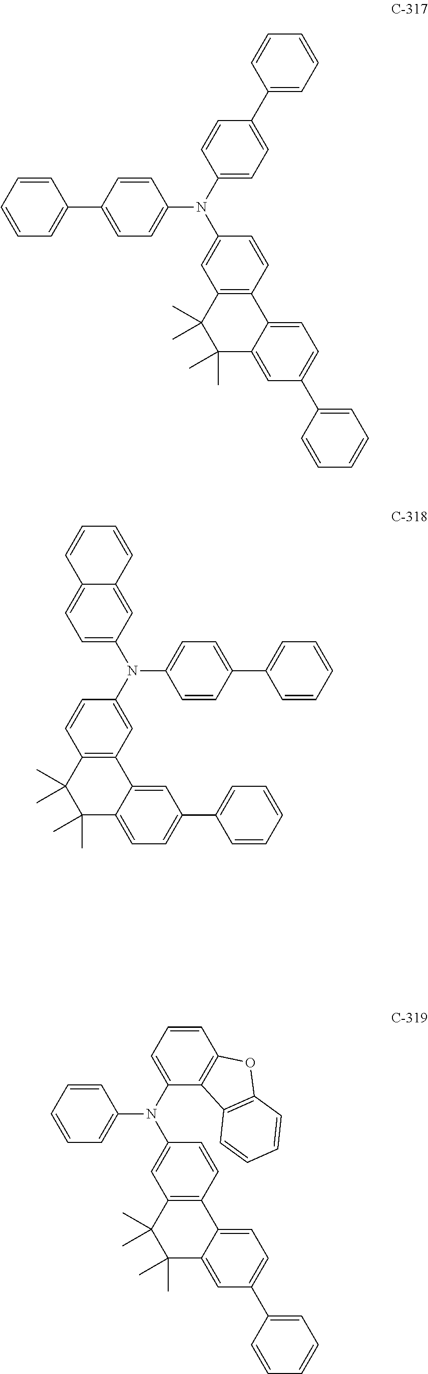 [SMILES: CC1(C)c2cc(-c3ccccc3)ccc2-c2ccc(N(c3ccc(-c4ccccc4)cc3)c3ccc(-c4ccccc4)cc3)cc2C1(C)C ; CC1(C)c2ccc(-c3ccccc3)cc2-c2cc(N(c3ccc(-c4ccccc4)cc3)c3ccc4ccccc4c3)ccc2C1(C)C ; CC1(C)c2cc(-c3ccccc3)ccc2-c2ccc(N(c3ccccc3)c3cccc4oc5ccccc5c34)cc2C1(C)C]

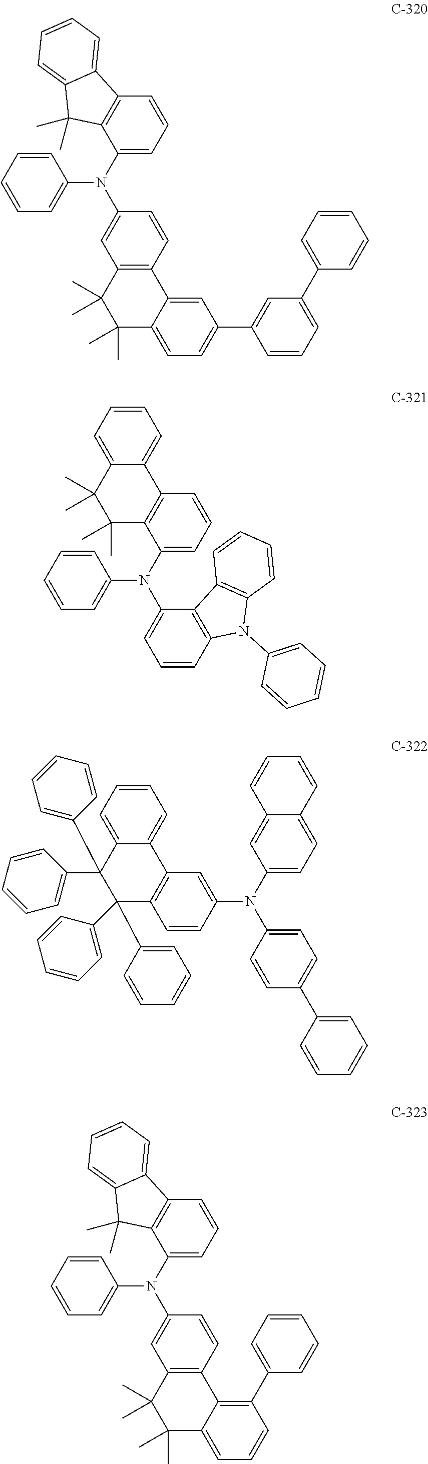 [SMILES: CC1(C)c2ccccc2-c2cccc(N(c3ccccc3)c3ccc4c(c3)C(C)(C)C(C)(C)c3ccc(-c5cccc(-c6ccccc6)c5)cc3-4)c21 ; CC1(C)c2ccccc2-c2cccc(N(c3ccccc3)c3cccc4c3c3ccccc3n4-c3ccccc3)c2C1(C)C ; c1ccc(-c2ccc(N(c3ccc4c(c3)-c3ccccc3C(c3ccccc3)(c3ccccc3)C4(c3ccccc3)c3ccccc3)c3ccc4ccccc4c3)cc2)cc1 ; CC1(C)c2ccccc2-c2cccc(N(c3ccccc3)c3ccc4c(c3)C(C)(C)C(C)(C)c3cccc(-c5ccccc5)c3-4)c21]

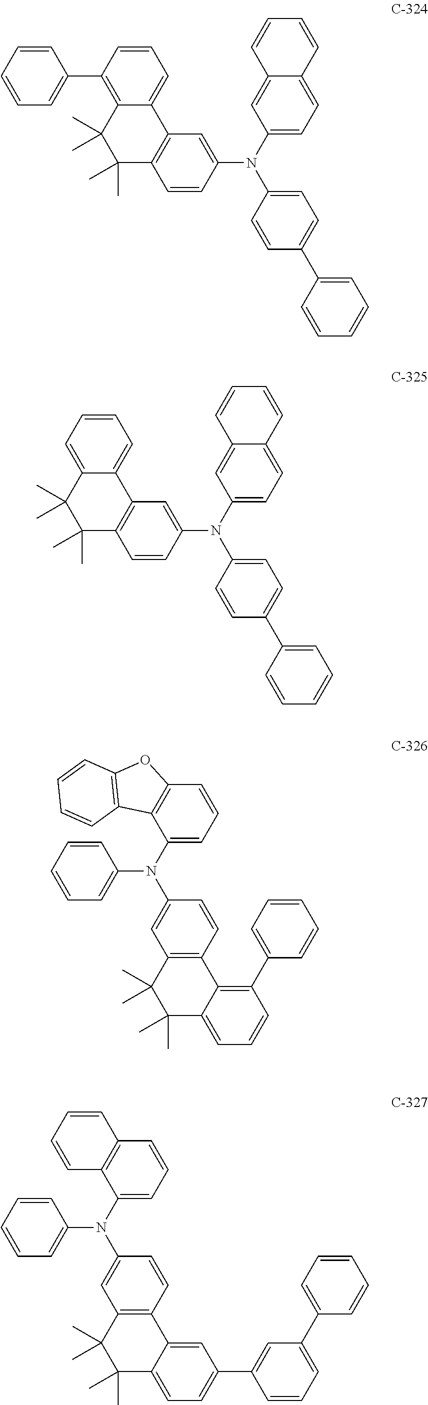 [SMILES: CC1(C)c2ccc(N(c3ccc(-c4ccccc4)cc3)c3ccc4ccccc4c3)cc2-c2cccc(-c3ccccc3)c2C1(C)C ; CC1(C)c2ccccc2-c2cc(N(c3ccc(-c4ccccc4)cc3)c3ccc4ccccc4c3)ccc2C1(C)C ; CC1(C)c2cc(N(c3ccccc3)c3cccc4oc5ccccc5c34)ccc2-c2c(-c3ccccc3)cccc2C1(C)C ; CC1(C)c2ccc(-c3cccc(-c4ccccc4)c3)cc2-c2ccc(N(c3ccccc3)c3cccc4ccccc34)cc2C1(C)C]

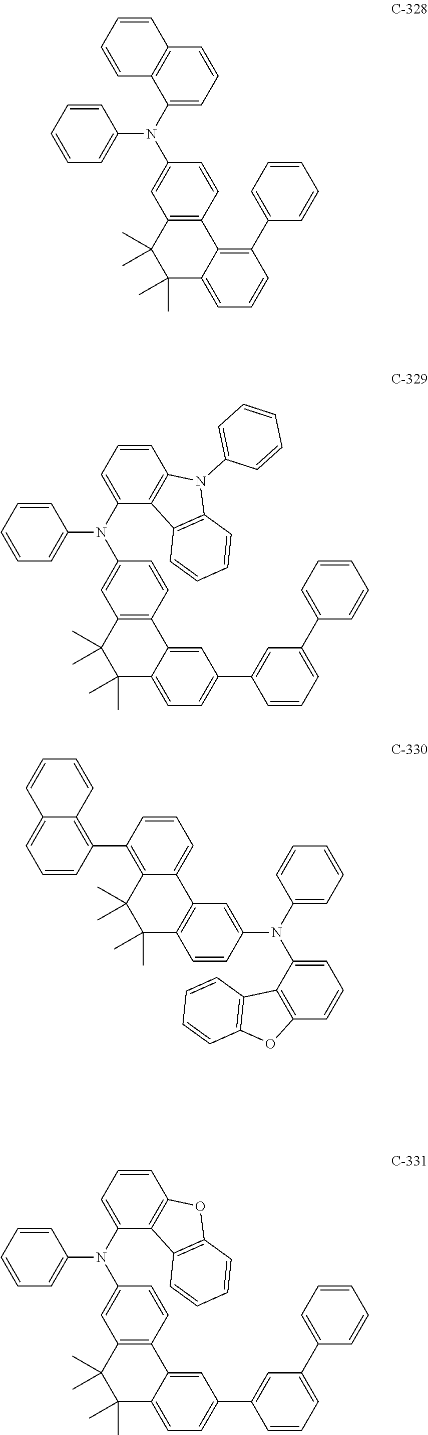 [SMILES: CC1(C)c2cc(N(c3ccccc3)c3cccc4ccccc34)ccc2-c2c(-c3ccccc3)cccc2C1(C)C ; CC1(C)c2ccc(-c3cccc(-c4ccccc4)c3)cc2-c2ccc(N(c3ccccc3)c3cccc4c3c3ccccc3n4-c3ccccc3)cc2C1(C)C ; CC1(C)c2ccc(N(c3ccccc3)c3cccc4oc5ccccc5c34)cc2-c2cccc(-c3cccc4ccccc34)c2C1(C)C ; CC1(C)c2ccc(-c3cccc(-c4ccccc4)c3)cc2-c2ccc(N(c3ccccc3)c3cccc4oc5ccccc5c34)cc2C1(C)C]

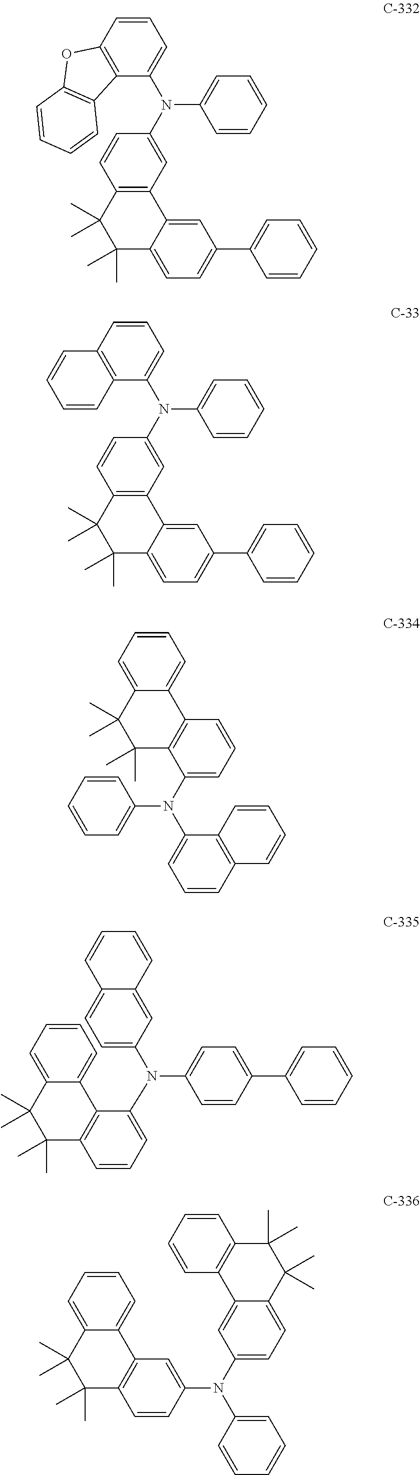

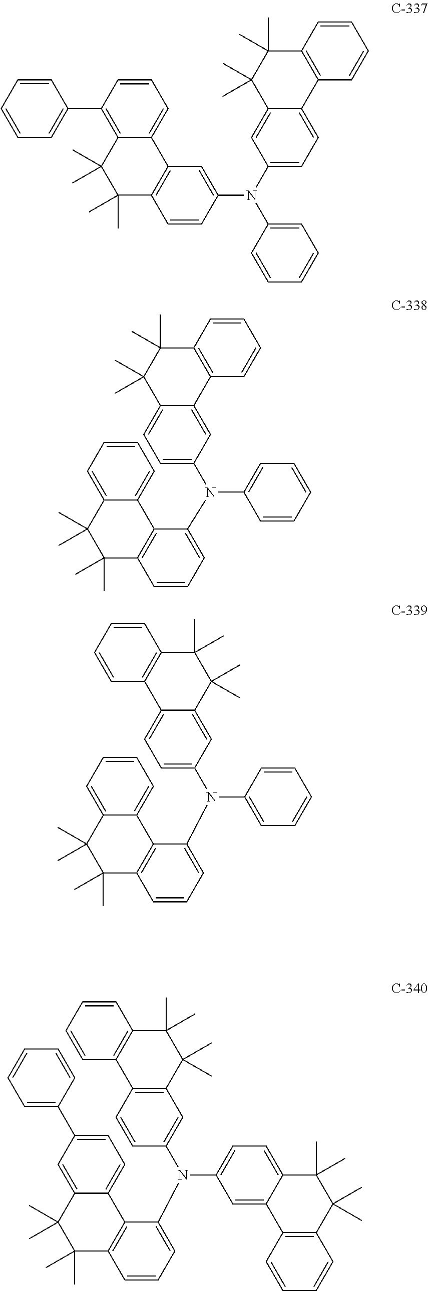

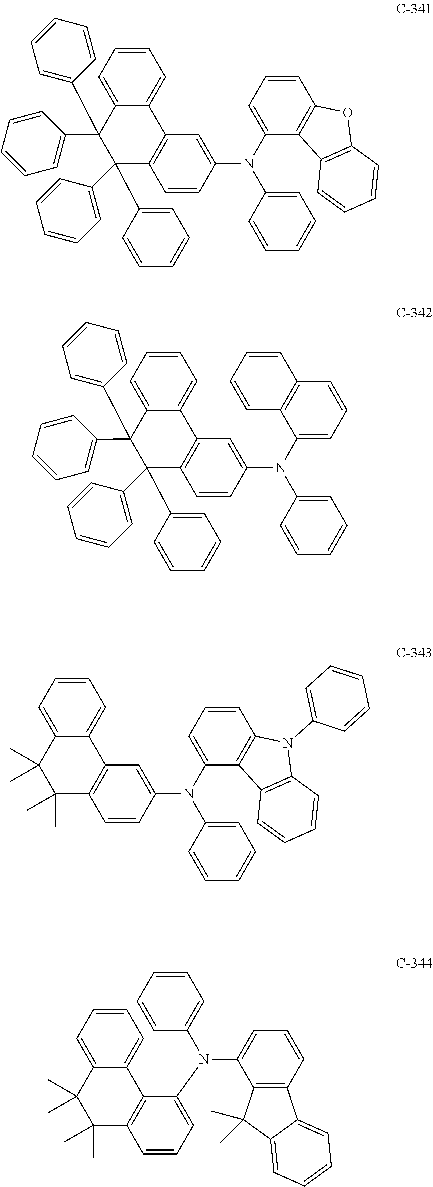

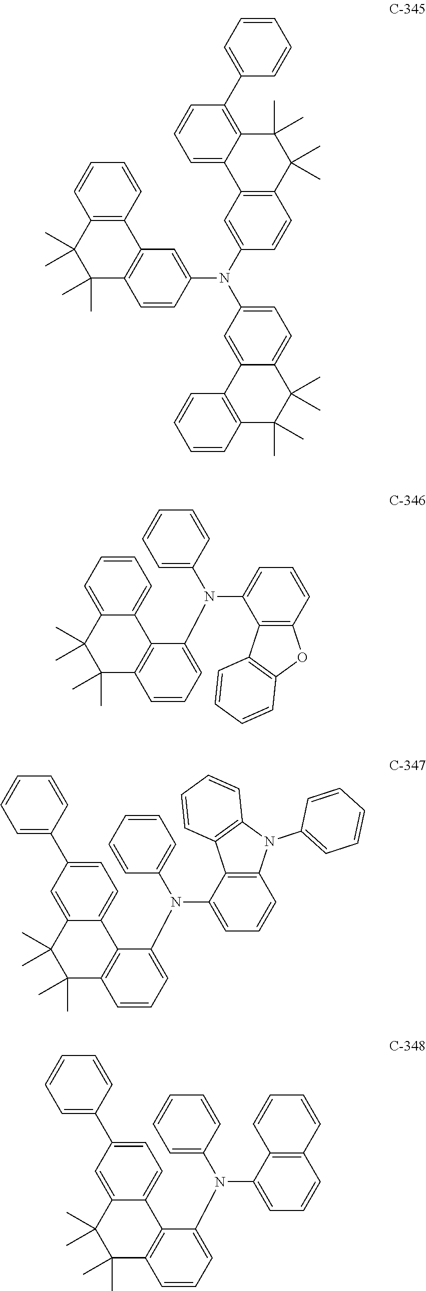

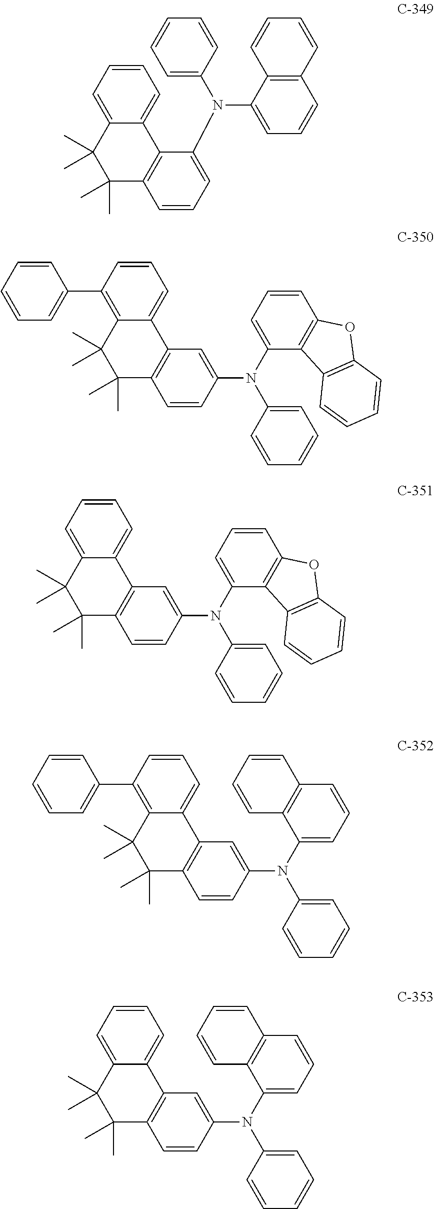 [SMILES: CC1(C)c2ccccc2-c2c(N(c3ccccc3)c3cccc4ccccc34)cccc2C1(C)C ; CC1(C)c2ccc(N(c3ccccc3)c3cccc4oc5ccccc5c34)cc2-c2cccc(-c3ccccc3)c2C1(C)C ; CC1(C)c2ccccc2-c2cc(N(c3ccccc3)c3cccc4oc5ccccc5c34)ccc2C1(C)C ; CC1(C)c2ccc(N(c3ccccc3)c3cccc4ccccc34)cc2-c2cccc(-c3ccccc3)c2C1(C)C ; CC1(C)c2ccccc2-c2cc(N(c3ccccc3)c3cccc4ccccc34)ccc2C1(C)C]

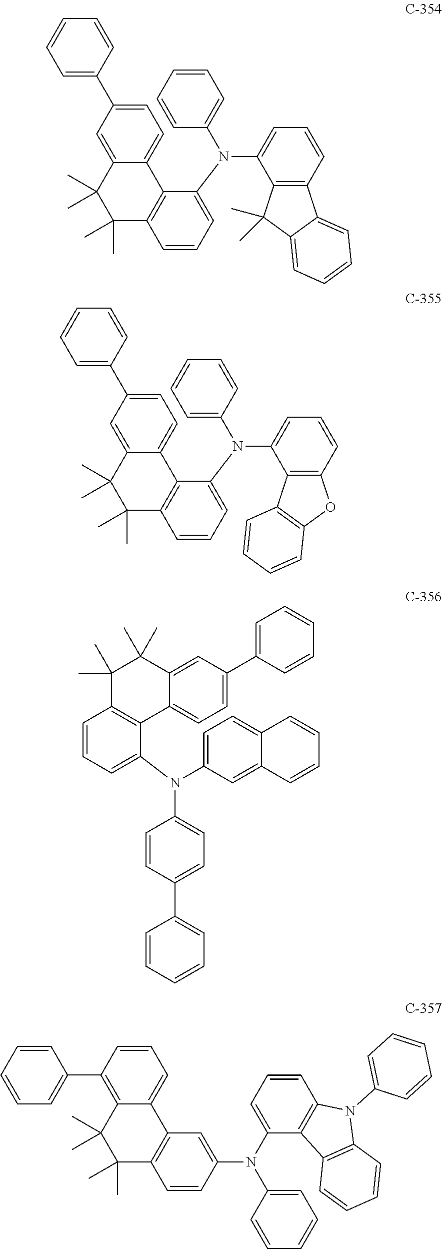

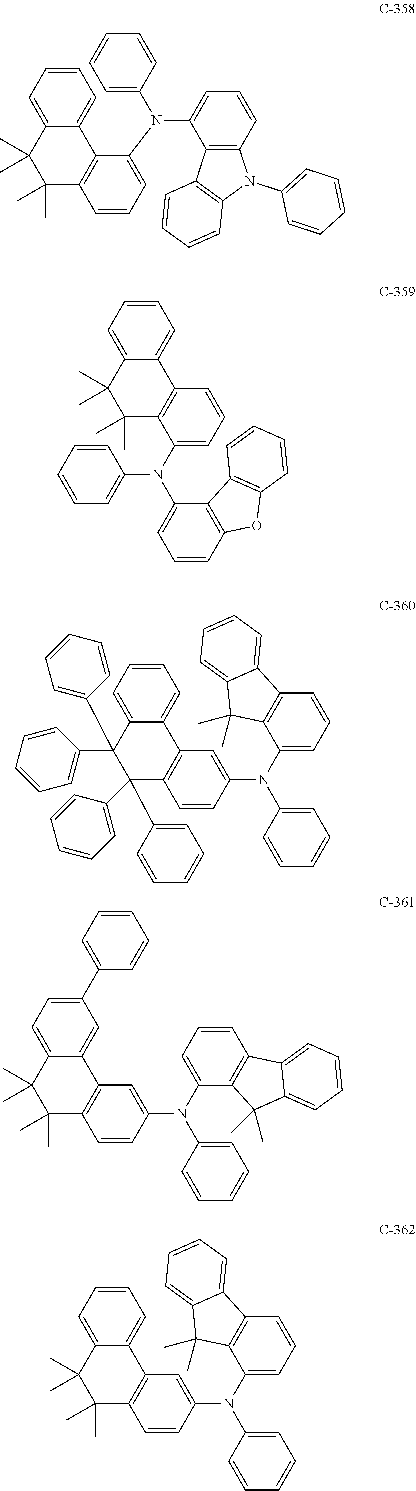

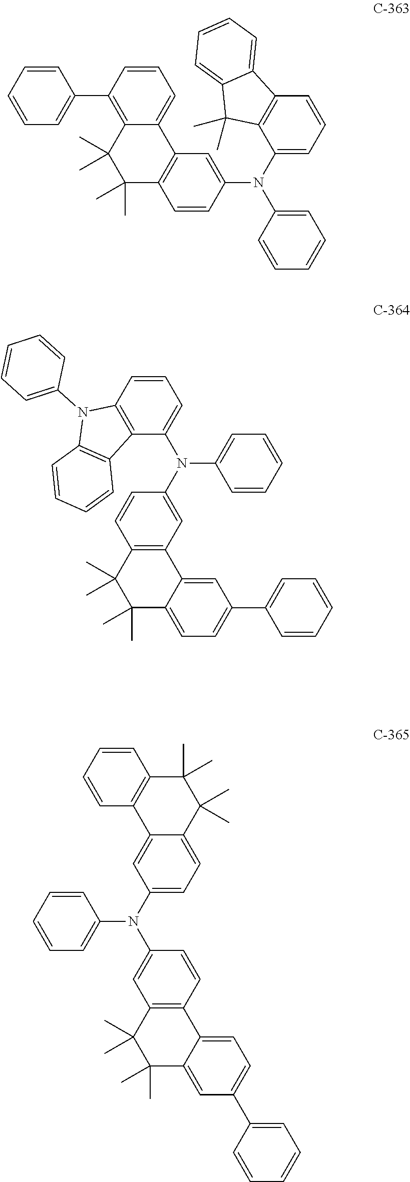 [SMILES: CC1(C)c2ccccc2-c2cccc(N(c3ccccc3)c3ccc4c(c3)-c3cccc(-c5ccccc5)c3C(C)(C)C4(C)C)c21 ; CC1(C)c2ccc(-c3ccccc3)cc2-c2cc(N(c3ccccc3)c3cccc4c3c3ccccc3n4-c3ccccc3)ccc2C1(C)C ; CC1(C)c2ccccc2-c2cc(N(c3ccccc3)c3ccc4c(c3)C(C)(C)C(C)(C)c3cc(-c5ccccc5)ccc3-4)ccc2C1(C)C]

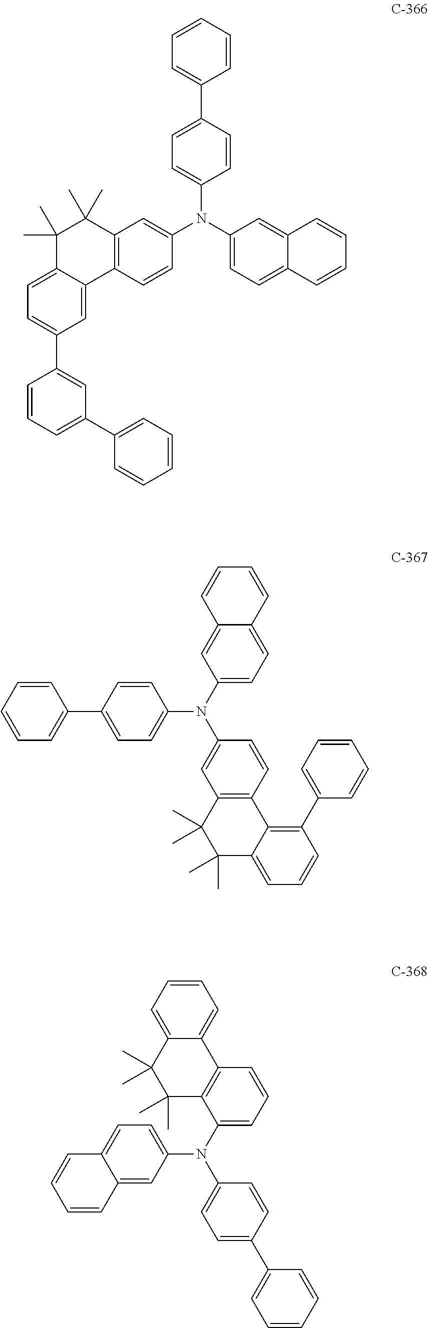 [SMILES: CC1(C)c2ccc(-c3cccc(-c4ccccc4)c3)cc2-c2ccc(N(c3ccc(-c4ccccc4)cc3)c3ccc4ccccc4c3)cc2C1(C)C ; CC1(C)c2cc(N(c3ccc(-c4ccccc4)cc3)c3ccc4ccccc4c3)ccc2-c2c(-c3ccccc3)cccc2C1(C)C ; CC1(C)c2ccccc2-c2cccc(N(c3ccc(-c4ccccc4)cc3)c3ccc4ccccc4c3)c2C1(C)C]

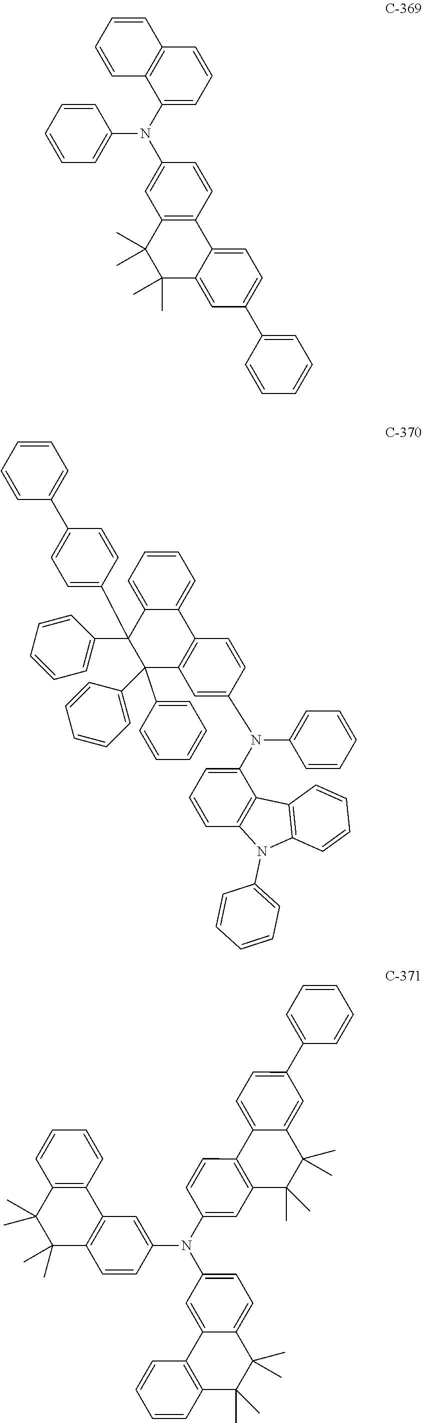

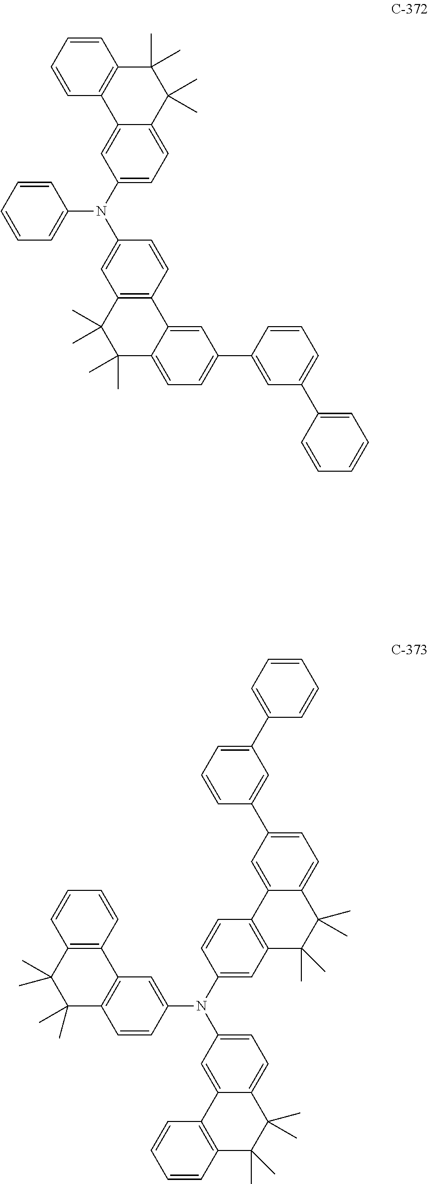

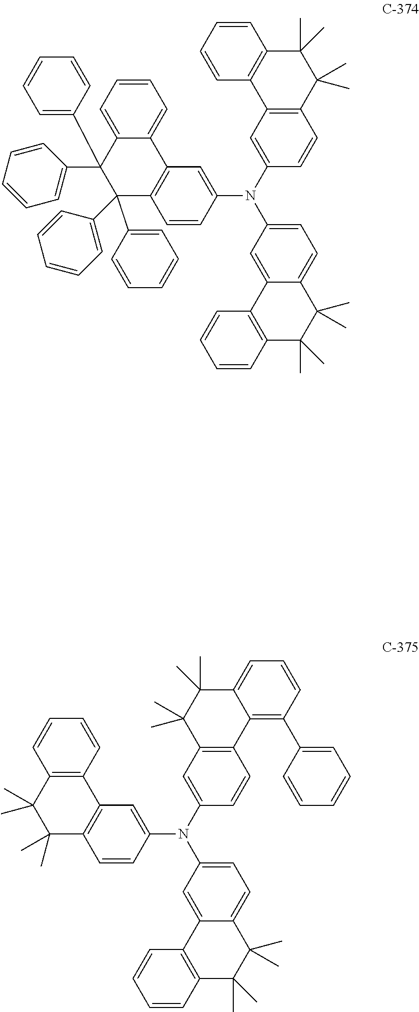

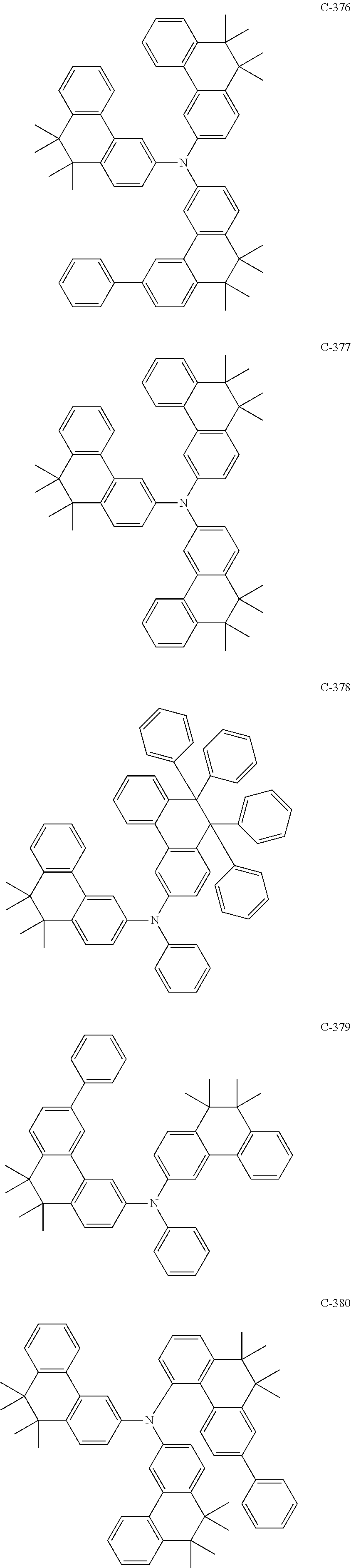

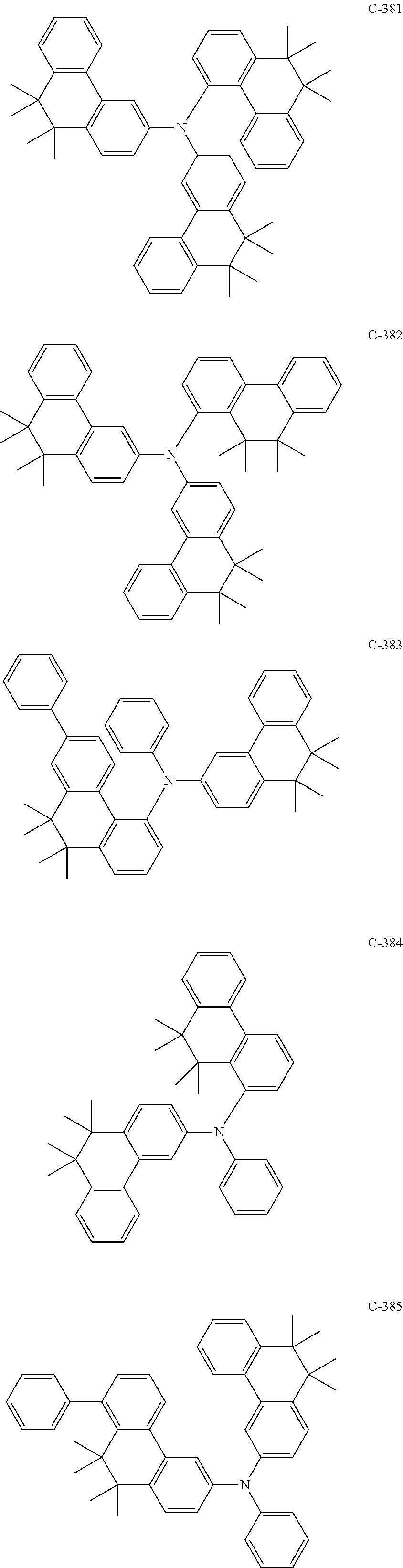

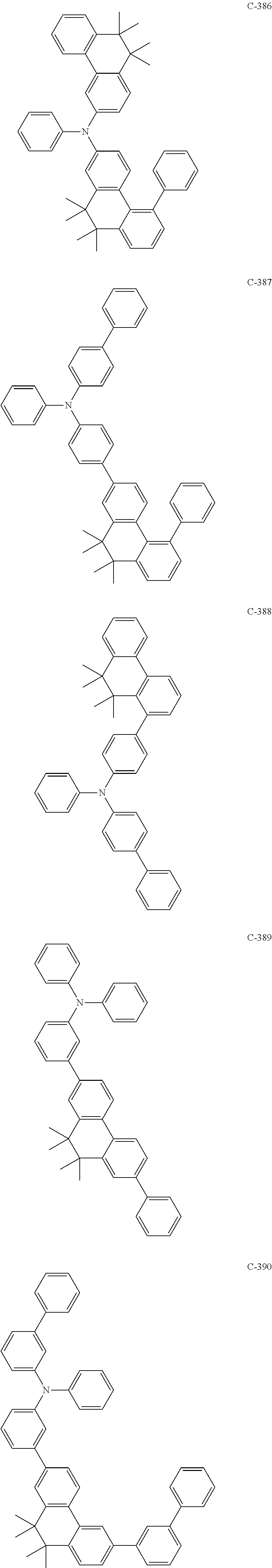 [SMILES: CC1(C)c2ccccc2-c2cc(N(c3ccccc3)c3ccc4c(c3)C(C)(C)C(C)(C)c3cccc(-c5ccccc5)c3-4)ccc2C1(C)C ; CC1(C)c2cc(-c3ccc(N(c4ccccc4)c4ccc(-c5ccccc5)cc4)cc3)ccc2-c2c(-c3ccccc3)cccc2C1(C)C ; CC1(C)c2ccccc2-c2cccc(-c3ccc(N(c4ccccc4)c4ccc(-c5ccccc5)cc4)cc3)c2C1(C)C ; CC1(C)c2cc(-c3ccccc3)ccc2-c2ccc(-c3cccc(N(c4ccccc4)c4ccccc4)c3)cc2C1(C)C ; CC1(C)c2ccc(-c3cccc(-c4ccccc4)c3)cc2-c2ccc(-c3cccc(N(c4ccccc4)c4cccc(-c5ccccc5)c4)c3)cc2C1(C)C]

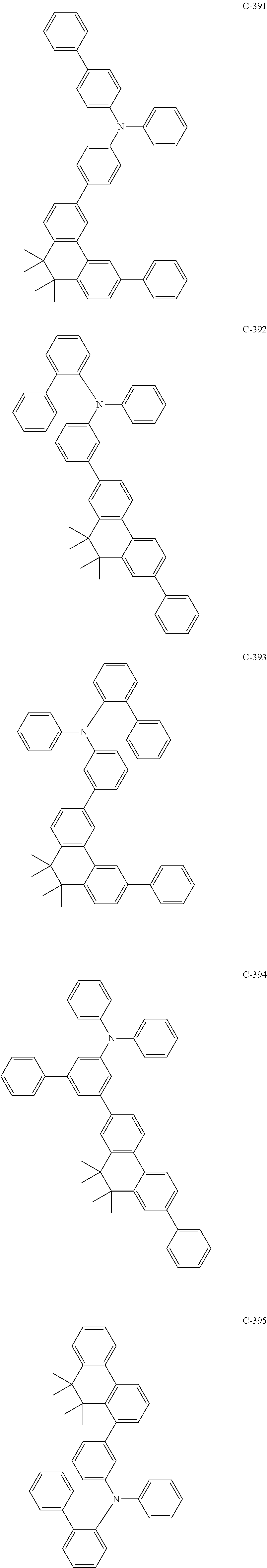 [SMILES: CC1(C)c2ccc(-c3ccccc3)cc2-c2cc(-c3ccc(N(c4ccccc4)c4ccc(-c5ccccc5)cc4)cc3)ccc2C1(C)C ; CC1(C)c2cc(-c3ccccc3)ccc2-c2ccc(-c3cccc(N(c4ccccc4)c4ccccc4-c4ccccc4)c3)cc2C1(C)C ; CC1(C)c2ccc(-c3ccccc3)cc2-c2cc(-c3cccc(N(c4ccccc4)c4ccccc4-c4ccccc4)c3)ccc2C1(C)C ; CC1(C)c2cc(-c3ccccc3)ccc2-c2ccc(-c3cc(-c4ccccc4)cc(N(c4ccccc4)c4ccccc4)c3)cc2C1(C)C ; CC1(C)c2ccccc2-c2cccc(-c3cccc(N(c4ccccc4)c4ccccc4-c4ccccc4)c3)c2C1(C)C]

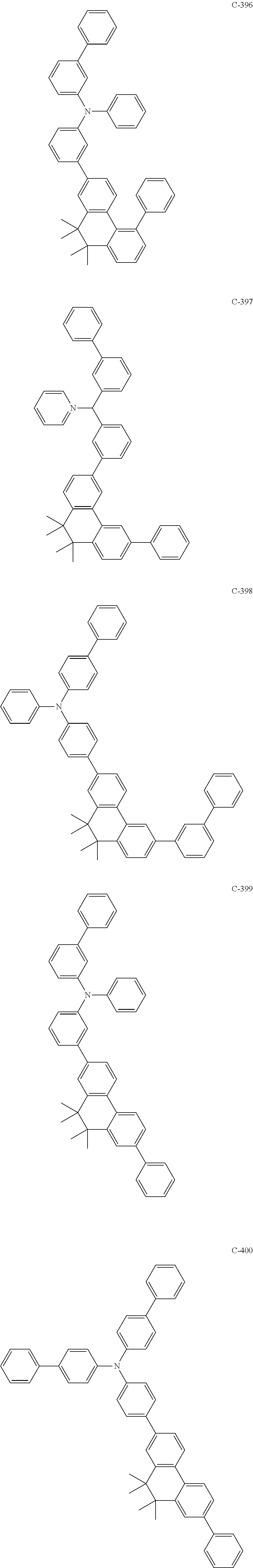

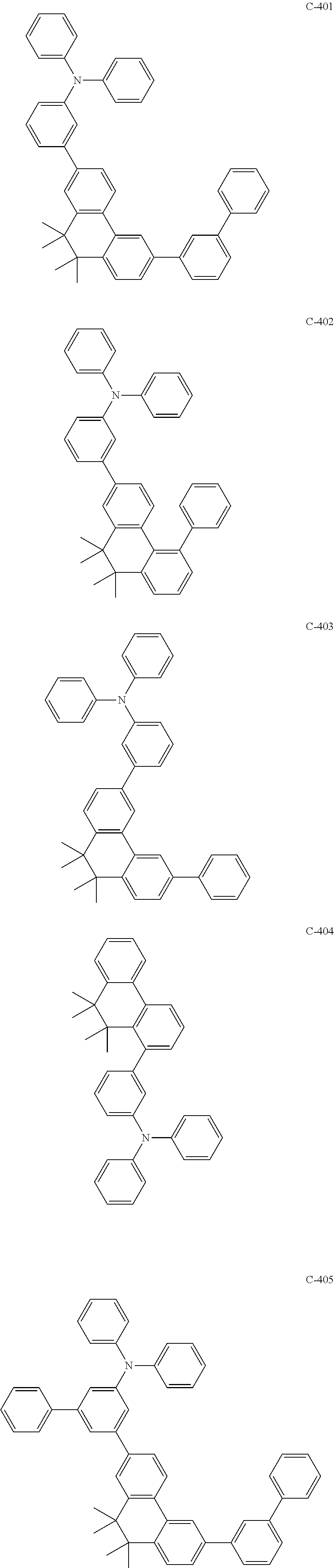 [SMILES: CC1(C)c2ccc(-c3cccc(-c4ccccc4)c3)cc2-c2ccc(-c3cccc(N(c4ccccc4)c4ccccc4)c3)cc2C1(C)C ; CC1(C)c2cc(-c3cccc(N(c4ccccc4)c4ccccc4)c3)ccc2-c2c(-c3ccccc3)cccc2C1(C)C ; CC1(C)c2ccc(-c3ccccc3)cc2-c2cc(-c3cccc(N(c4ccccc4)c4ccccc4)c3)ccc2C1(C)C ; CC1(C)c2ccccc2-c2cccc(-c3cccc(N(c4ccccc4)c4ccccc4)c3)c2C1(C)C ; CC1(C)c2ccc(-c3cccc(-c4ccccc4)c3)cc2-c2ccc(-c3cc(-c4ccccc4)cc(N(c4ccccc4)c4ccccc4)c3)cc2C1(C)C]

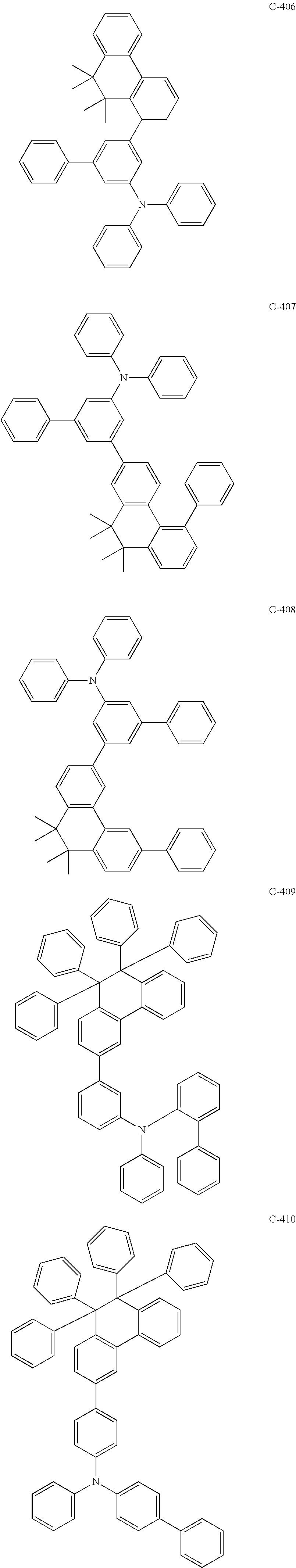

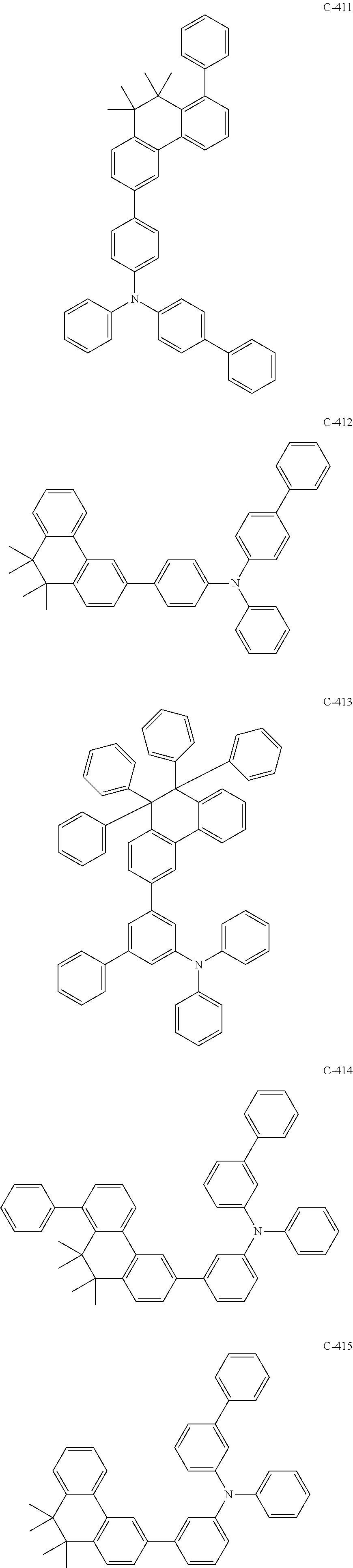 [SMILES: CC1(C)c2ccc(-c3ccc(N(c4ccccc4)c4ccc(-c5ccccc5)cc4)cc3)cc2-c2cccc(-c3ccccc3)c2C1(C)C ; CC1(C)c2ccccc2-c2cc(-c3ccc(N(c4ccccc4)c4ccc(-c5ccccc5)cc4)cc3)ccc2C1(C)C ; c1ccc(-c2cc(-c3ccc4c(c3)-c3ccccc3C(c3ccccc3)(c3ccccc3)C4(c3ccccc3)c3ccccc3)cc(N(c3ccccc3)c3ccccc3)c2)cc1 ; CC1(C)c2ccc(-c3cccc(N(c4ccccc4)c4cccc(-c5ccccc5)c4)c3)cc2-c2cccc(-c3ccccc3)c2C1(C)C ; CC1(C)c2ccccc2-c2cc(-c3cccc(N(c4ccccc4)c4cccc(-c5ccccc5)c4)c3)ccc2C1(C)C]

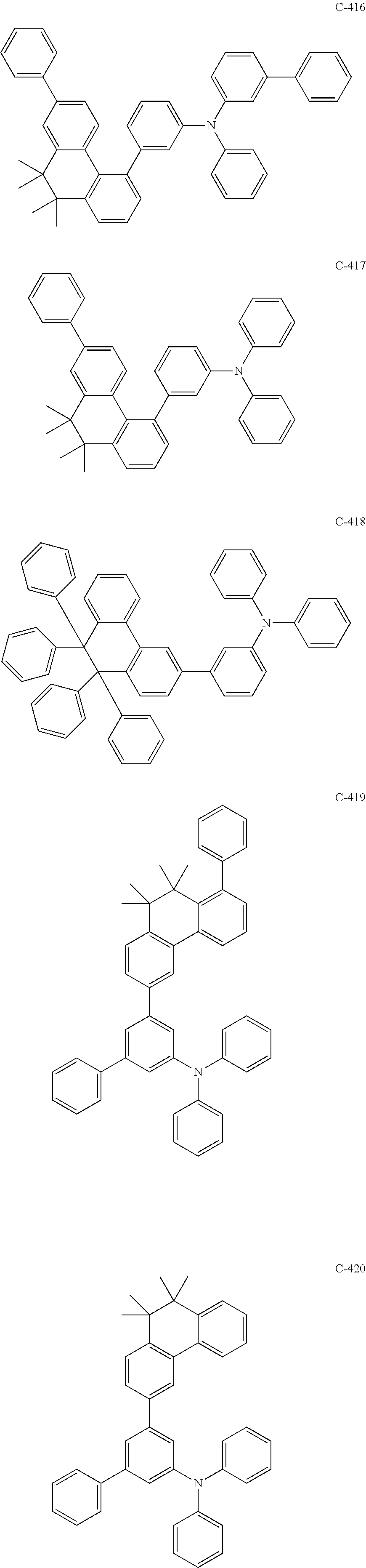 [SMILES: CC1(C)c2cc(-c3ccccc3)ccc2-c2c(-c3cccc(N(c4ccccc4)c4cccc(-c5ccccc5)c4)c3)cccc2C1(C)C ; CC1(C)c2cc(-c3ccccc3)ccc2-c2c(-c3cccc(N(c4ccccc4)c4ccccc4)c3)cccc2C1(C)C ; c1ccc(N(c2ccccc2)c2cccc(-c3ccc4c(c3)-c3ccccc3C(c3ccccc3)(c3ccccc3)C4(c3ccccc3)c3ccccc3)c2)cc1 ; CC1(C)c2ccc(-c3cc(-c4ccccc4)cc(N(c4ccccc4)c4ccccc4)c3)cc2-c2cccc(-c3ccccc3)c2C1(C)C ; CC1(C)c2ccccc2-c2cc(-c3cc(-c4ccccc4)cc(N(c4ccccc4)c4ccccc4)c3)ccc2C1(C)C]

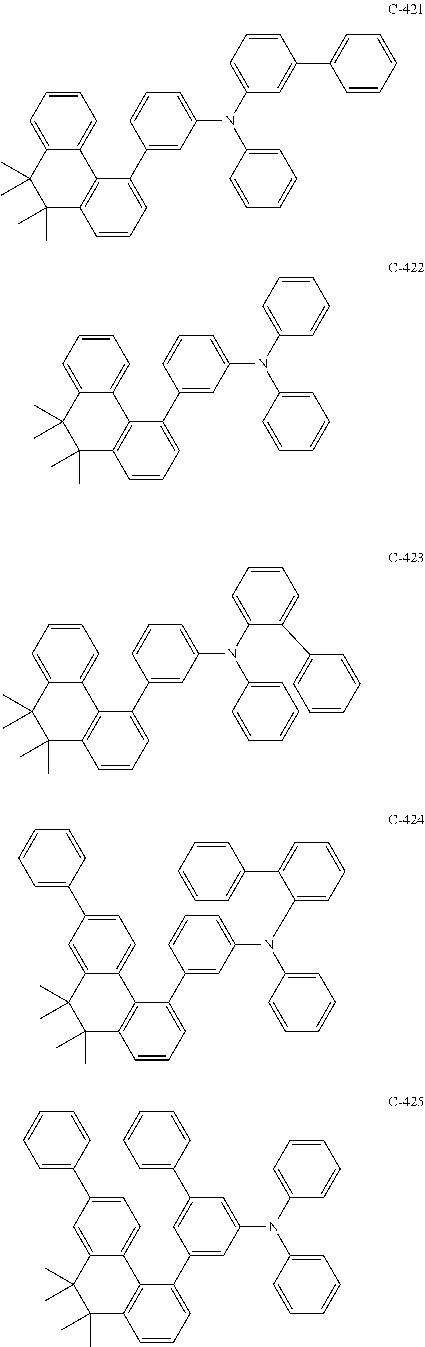 [SMILES: CC1(C)c2ccccc2-c2c(-c3cccc(N(c4ccccc4)c4cccc(-c5ccccc5)c4)c3)cccc2C1(C)C ; CC1(C)c2ccccc2-c2c(-c3cccc(N(c4ccccc4)c4ccccc4)c3)cccc2C1(C)C ; CC1(C)c2ccccc2-c2c(-c3cccc(N(c4ccccc4)c4ccccc4-c4ccccc4)c3)cccc2C1(C)C ; CC1(C)c2cc(-c3ccccc3)ccc2-c2c(-c3cccc(N(c4ccccc4)c4ccccc4-c4ccccc4)c3)cccc2C1(C)C ; CC1(C)c2cc(-c3ccccc3)ccc2-c2c(-c3cc(-c4ccccc4)cc(N(c4ccccc4)c4ccccc4)c3)cccc2C1(C)C]

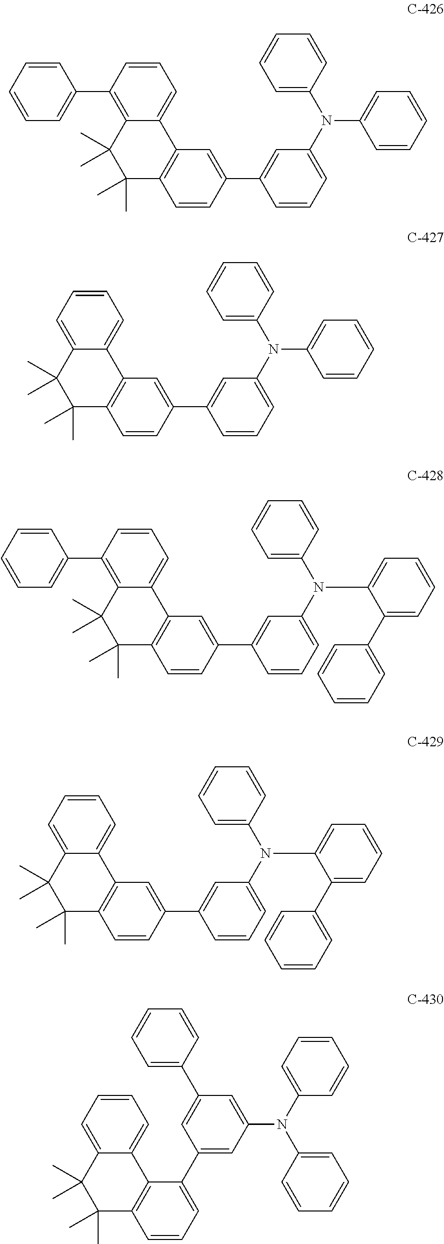 [SMILES: CC1(C)c2ccc(-c3cccc(N(c4ccccc4)c4ccccc4)c3)cc2-c2cccc(-c3ccccc3)c2C1(C)C ; CC1(C)c2ccccc2-c2cc(-c3cccc(N(c4ccccc4)c4ccccc4)c3)ccc2C1(C)C ; CC1(C)c2ccc(-c3cccc(N(c4ccccc4)c4ccccc4-c4ccccc4)c3)cc2-c2cccc(-c3ccccc3)c2C1(C)C ; CC1(C)c2ccccc2-c2cc(-c3cccc(N(c4ccccc4)c4ccccc4-c4ccccc4)c3)ccc2C1(C)C ; CC1(C)c2ccccc2-c2c(-c3cc(-c4ccccc4)cc(N(c4ccccc4)c4ccccc4)c3)cccc2C1(C)C]

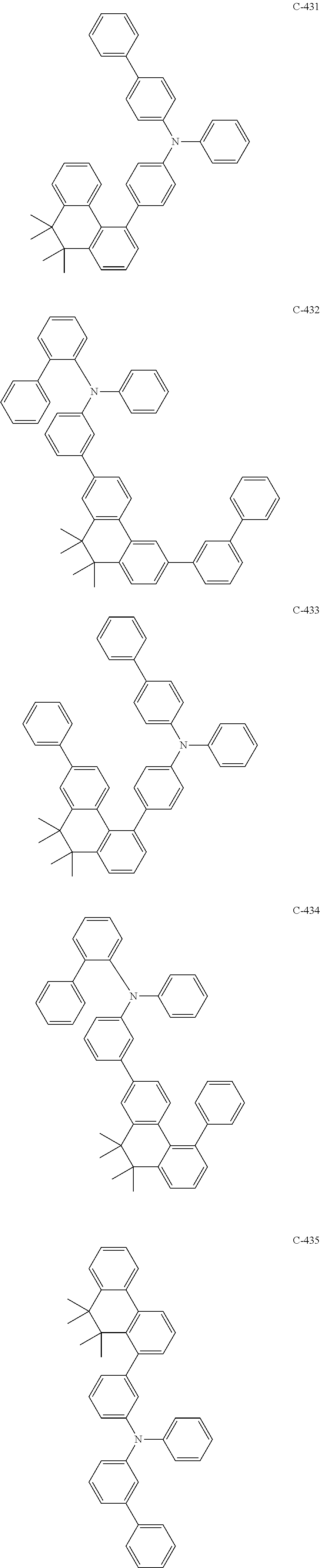

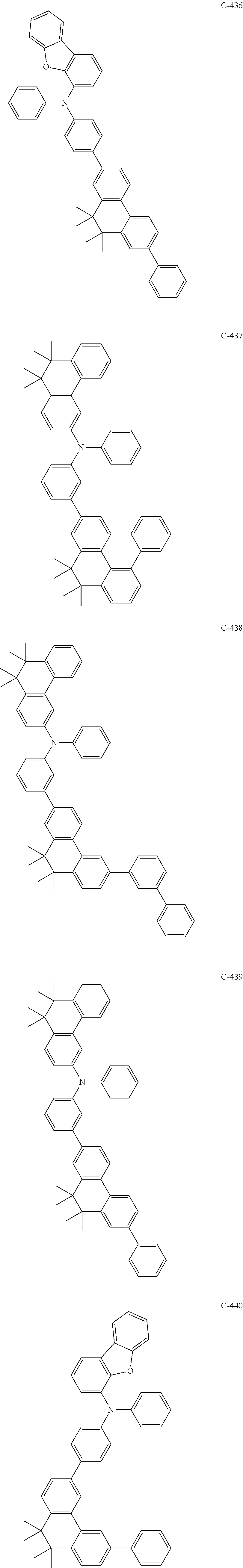 [SMILES: CC1(C)c2cc(-c3ccccc3)ccc2-c2ccc(-c3ccc(N(c4ccccc4)c4cccc5c4oc4ccccc45)cc3)cc2C1(C)C ; CC1(C)c2ccccc2-c2cc(N(c3ccccc3)c3cccc(-c4ccc5c(c4)C(C)(C)C(C)(C)c4cccc(-c6ccccc6)c4-5)c3)ccc2C1(C)C ; CC1(C)c2ccccc2-c2cc(N(c3ccccc3)c3cccc(-c4ccc5c(c4)C(C)(C)C(C)(C)c4ccc(-c6cccc(-c7ccccc7)c6)cc4-5)c3)ccc2C1(C)C ; CC1(C)c2ccccc2-c2cc(N(c3ccccc3)c3cccc(-c4ccc5c(c4)C(C)(C)C(C)(C)c4cc(-c6ccccc6)ccc4-5)c3)ccc2C1(C)C ; CC1(C)c2ccc(-c3ccccc3)cc2-c2cc(-c3ccc(N(c4ccccc4)c4cccc5c4oc4ccccc45)cc3)ccc2C1(C)C]

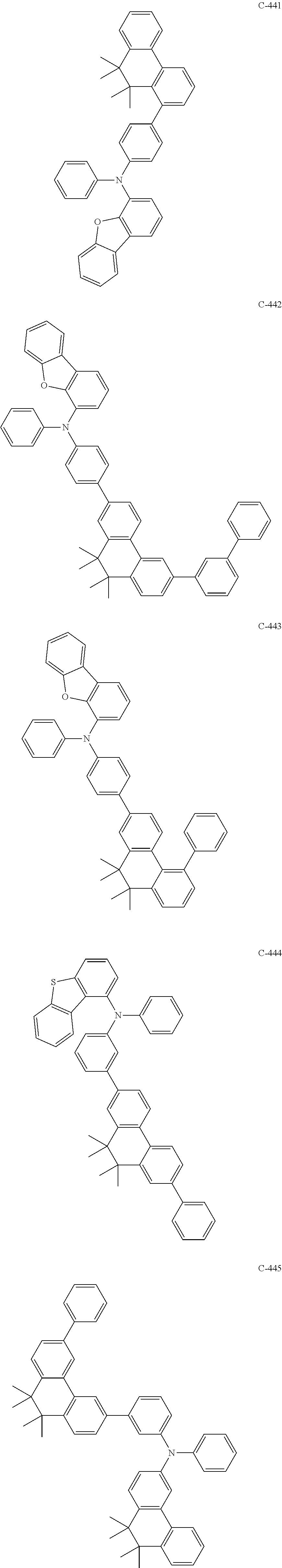 [SMILES: CC1(C)c2ccccc2-c2cccc(-c3ccc(N(c4ccccc4)c4cccc5c4oc4ccccc45)cc3)c2C1(C)C ; CC1(C)c2ccc(-c3cccc(-c4ccccc4)c3)cc2-c2ccc(-c3ccc(N(c4ccccc4)c4cccc5c4oc4ccccc45)cc3)cc2C1(C)C ; CC1(C)c2cc(-c3ccc(N(c4ccccc4)c4cccc5c4oc4ccccc45)cc3)ccc2-c2c(-c3ccccc3)cccc2C1(C)C ; CC1(C)c2cc(-c3ccccc3)ccc2-c2ccc(-c3cccc(N(c4ccccc4)c4cccc5sc6ccccc6c45)c3)cc2C1(C)C ; CC1(C)c2ccccc2-c2cc(N(c3ccccc3)c3cccc(-c4ccc5c(c4)-c4cc(-c6ccccc6)ccc4C(C)(C)C5(C)C)c3)ccc2C1(C)C]

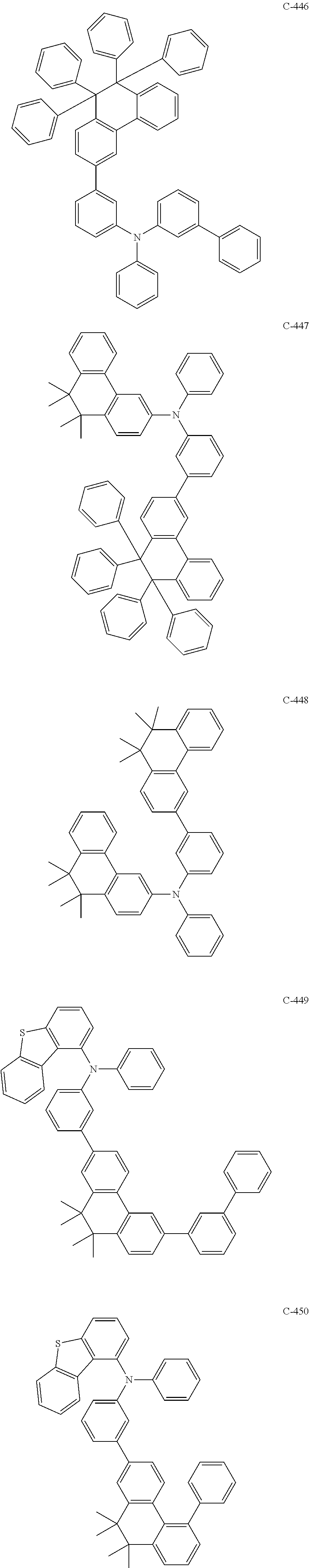

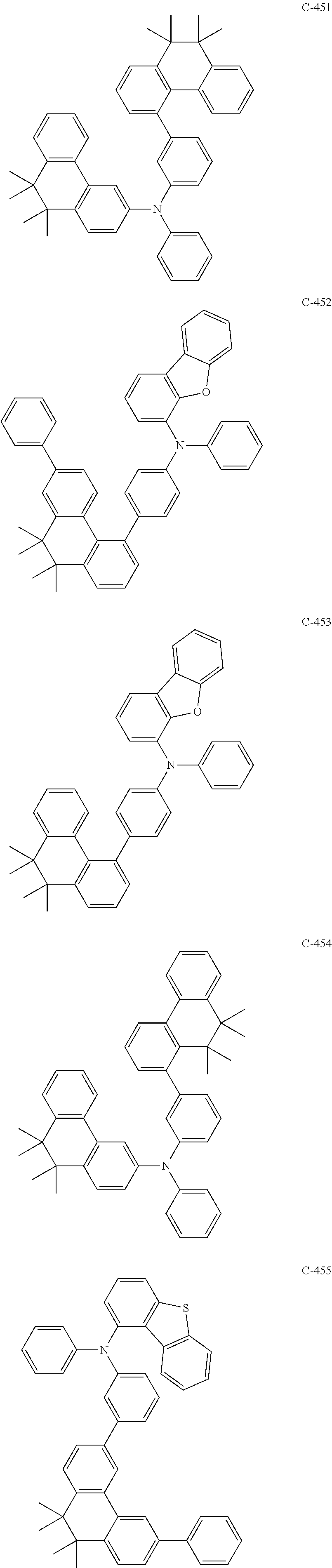

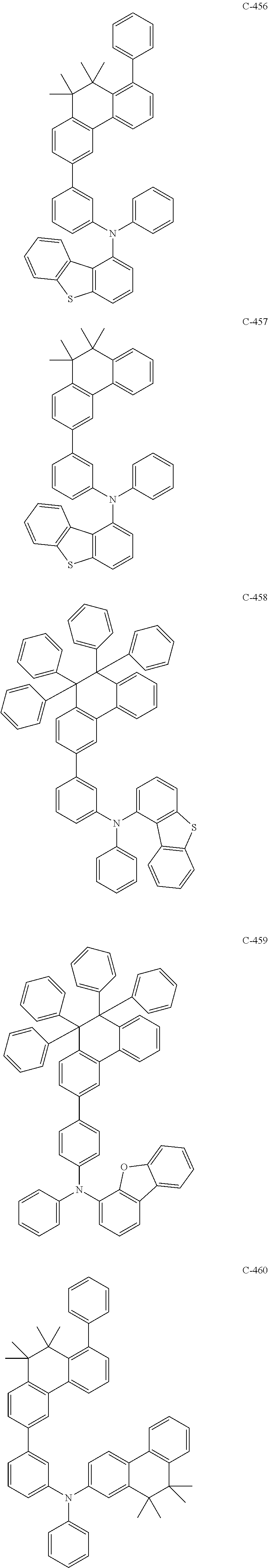

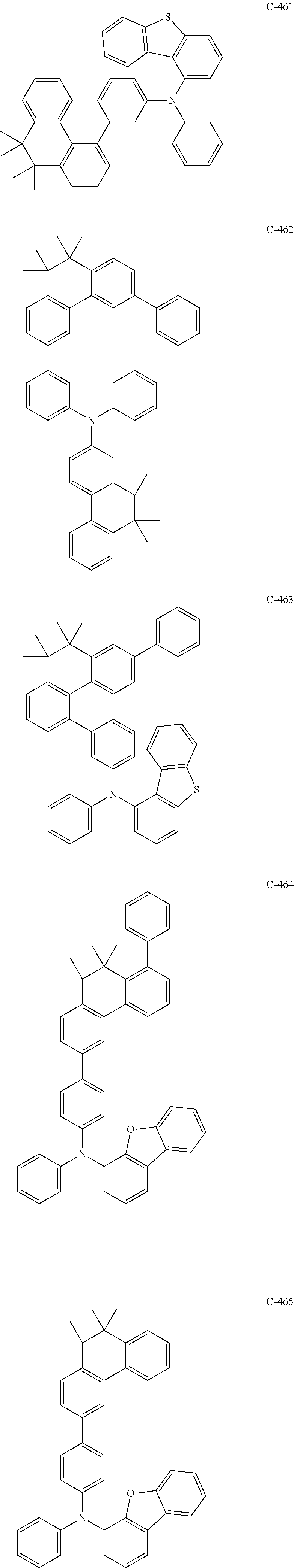 [SMILES: CC1(C)c2ccccc2-c2c(-c3cccc(N(c4ccccc4)c4cccc5sc6ccccc6c45)c3)cccc2C1(C)C ; CC1(C)c2ccc(-c3ccccc3)cc2-c2cc(-c3cccc(N(c4ccccc4)c4ccc5c(c4)C(C)(C)C(C)(C)c4ccccc4-5)c3)ccc2C1(C)C ; CC1(C)c2cc(-c3ccccc3)ccc2-c2c(-c3cccc(N(c4ccccc4)c4cccc5sc6ccccc6c45)c3)cccc2C1(C)C ; CC1(C)c2ccc(-c3ccc(N(c4ccccc4)c4cccc5c4oc4ccccc45)cc3)cc2-c2cccc(-c3ccccc3)c2C1(C)C ; CC1(C)c2ccccc2-c2cc(-c3ccc(N(c4ccccc4)c4cccc5c4oc4ccccc45)cc3)ccc2C1(C)C]

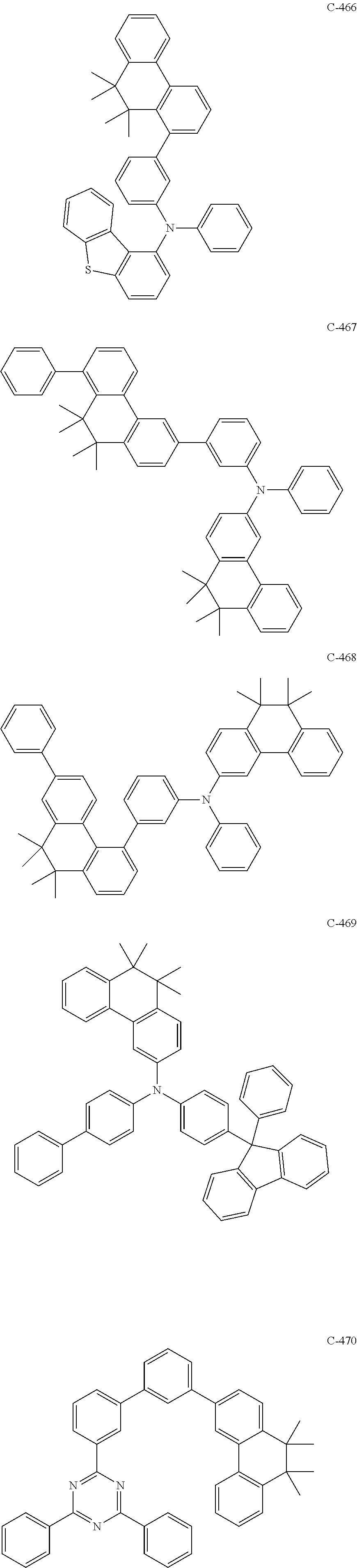 [SMILES: CC1(C)c2ccccc2-c2cccc(-c3cccc(N(c4ccccc4)c4cccc5sc6ccccc6c45)c3)c2C1(C)C ; CC1(C)c2ccccc2-c2cc(N(c3ccccc3)c3cccc(-c4ccc5c(c4)-c4cccc(-c6ccccc6)c4C(C)(C)C5(C)C)c3)ccc2C1(C)C ; CC1(C)c2ccccc2-c2cc(N(c3ccccc3)c3cccc(-c4cccc5c4-c4ccc(-c6ccccc6)cc4C(C)(C)C5(C)C)c3)ccc2C1(C)C ; CC1(C)c2ccccc2-c2cc(N(c3ccc(-c4ccccc4)cc3)c3ccc(C4(c5ccccc5)c5ccccc5-c5ccccc54)cc3)ccc2C1(C)C ; CC1(C)c2ccccc2-c2cc(-c3cccc(-c4cccc(-c5nc(-c6ccccc6)nc(-c6ccccc6)n5)c4)c3)ccc2C1(C)C]

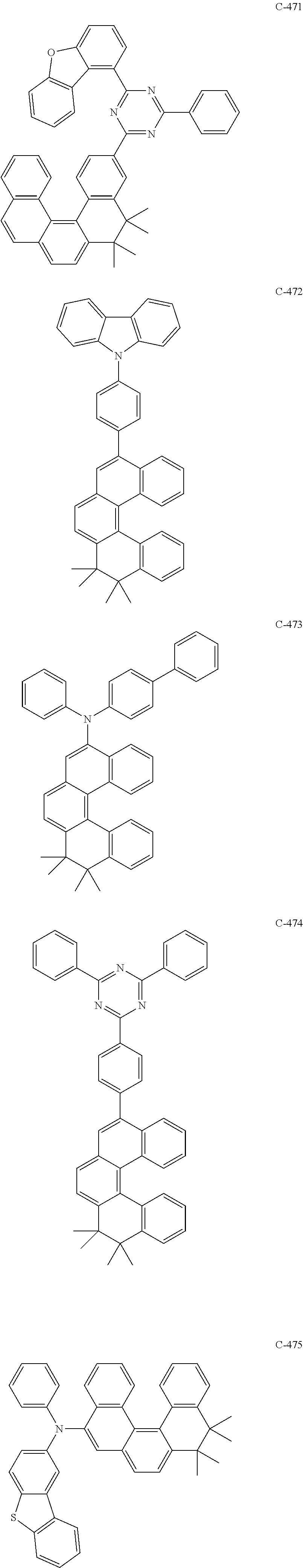 [SMILES: CC1(C)c2cc(-c3nc(-c4ccccc4)nc(-c4cccc5oc6ccccc6c45)n3)ccc2-c2c(ccc3ccc4ccccc4c23)C1(C)C ; CC1(C)c2ccccc2-c2c(ccc3cc(-c4ccc(-n5c6ccccc6c6ccccc65)cc4)c4ccccc4c23)C1(C)C ; CC1(C)c2ccccc2-c2c(ccc3cc(N(c4ccccc4)c4ccc(-c5ccccc5)cc4)c4ccccc4c23)C1(C)C ; CC1(C)c2ccccc2-c2c(ccc3cc(-c4ccc(-c5nc(-c6ccccc6)nc(-c6ccccc6)n5)cc4)c4ccccc4c23)C1(C)C ; CC1(C)c2ccccc2-c2c(ccc3cc(N(c4ccccc4)c4ccc5sc6ccccc6c5c4)c4ccccc4c23)C1(C)C]

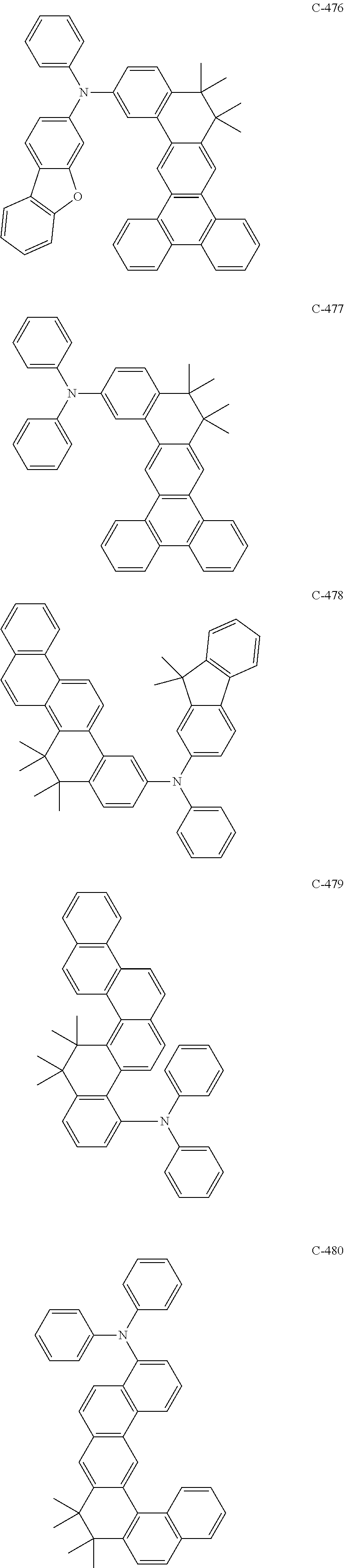 [SMILES: CC1(C)c2ccc(N(c3ccccc3)c3ccc4c(c3)oc3ccccc34)cc2-c2cc3c4ccccc4c4ccccc4c3cc2C1(C)C ; CC1(C)c2ccc(N(c3ccccc3)c3ccccc3)cc2-c2cc3c4ccccc4c4ccccc4c3cc2C1(C)C ; CC1(C)c2ccccc2-c2ccc(N(c3ccccc3)c3ccc4c(c3)-c3ccc5c(ccc6ccccc65)c3C(C)(C)C4(C)C)cc21 ; CC1(C)c2cccc(N(c3ccccc3)c3ccccc3)c2-c2ccc3ccc4c5ccccc5ccc4c3c2C1(C)C ; CC1(C)c2cc3ccc4c(N(c5ccccc5)c5ccccc5)cccc4c3cc2-c2c(ccc3ccccc23)C1(C)C]

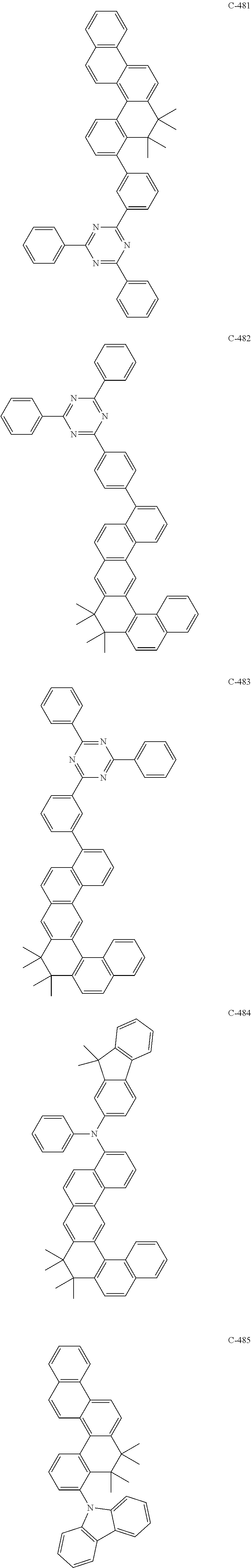

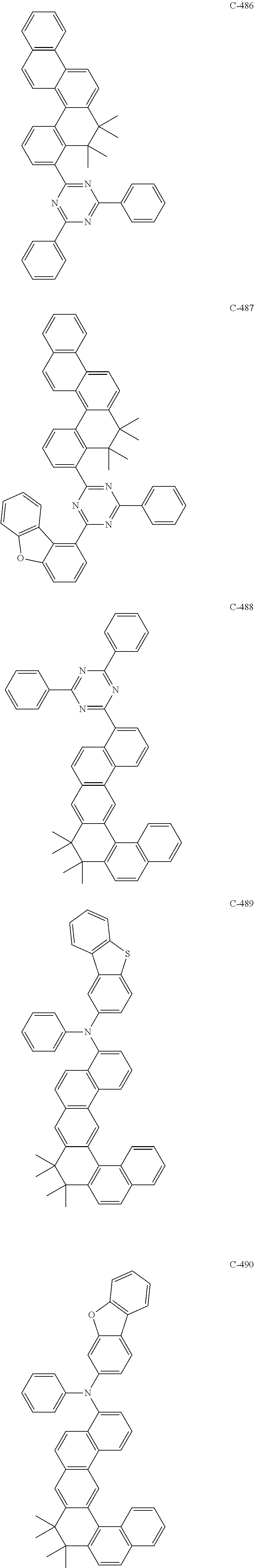

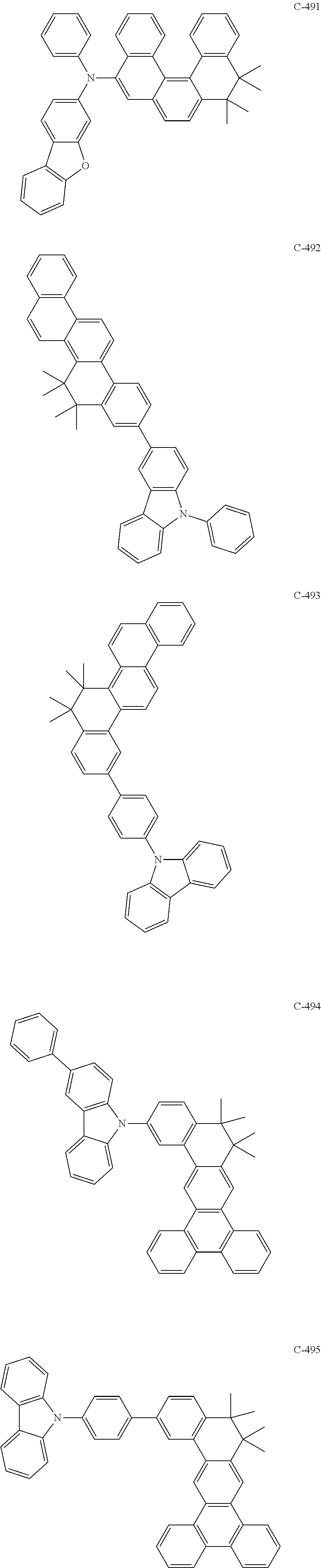

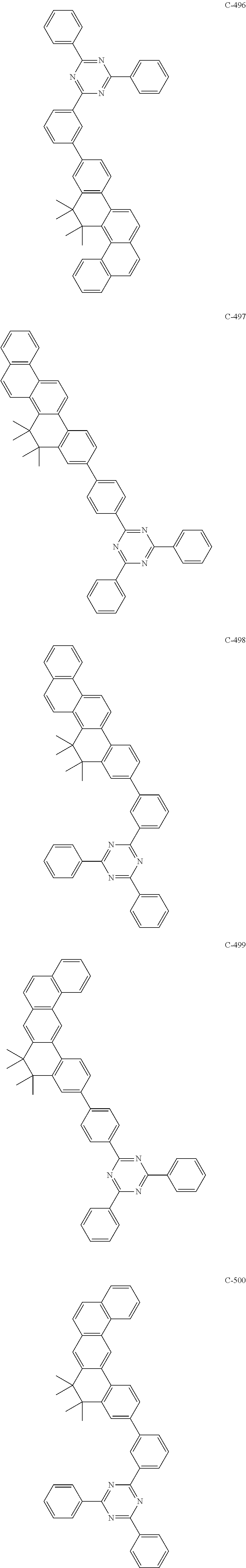 [SMILES: CC1(C)c2cc(-c3cccc(-c4nc(-c5ccccc5)nc(-c5ccccc5)n4)c3)ccc2-c2ccc3ccc4ccccc4c3c2C1(C)C ; CC1(C)c2cc(-c3ccc(-c4nc(-c5ccccc5)nc(-c5ccccc5)n4)cc3)ccc2-c2ccc3c(ccc4ccccc43)c2C1(C)C ; CC1(C)c2cc(-c3cccc(-c4nc(-c5ccccc5)nc(-c5ccccc5)n4)c3)ccc2-c2ccc3c(ccc4ccccc43)c2C1(C)C ; CC1(C)c2cc(-c3ccc(-c4nc(-c5ccccc5)nc(-c5ccccc5)n4)cc3)ccc2-c2cc3c(ccc4ccccc43)cc2C1(C)C ; CC1(C)c2cc(-c3cccc(-c4nc(-c5ccccc5)nc(-c5ccccc5)n4)c3)ccc2-c2cc3c(ccc4ccccc43)cc2C1(C)C]

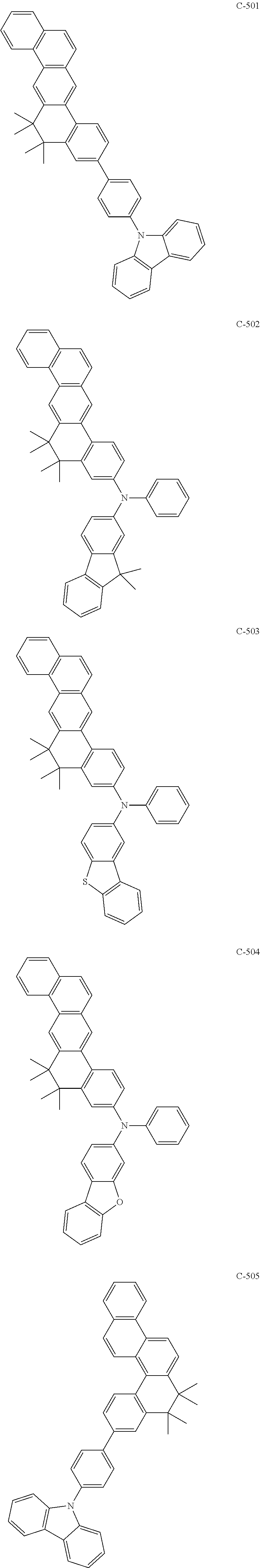 [SMILES: CC1(C)c2cc(-c3ccc(-n4c5ccccc5c5ccccc54)cc3)ccc2-c2cc3ccc4ccccc4c3cc2C1(C)C ; CC1(C)c2ccccc2-c2ccc(N(c3ccccc3)c3ccc4c(c3)C(C)(C)C(C)(C)c3cc5c(ccc6ccccc65)cc3-4)cc21 ; CC1(C)c2cc(N(c3ccccc3)c3ccc4sc5ccccc5c4c3)ccc2-c2cc3ccc4ccccc4c3cc2C1(C)C ; CC1(C)c2cc(N(c3ccccc3)c3ccc4c(c3)oc3ccccc34)ccc2-c2cc3ccc4ccccc4c3cc2C1(C)C ; CC1(C)c2cc(-c3ccc(-n4c5ccccc5c5ccccc54)cc3)ccc2-c2c(ccc3c2ccc2ccccc23)C1(C)C]

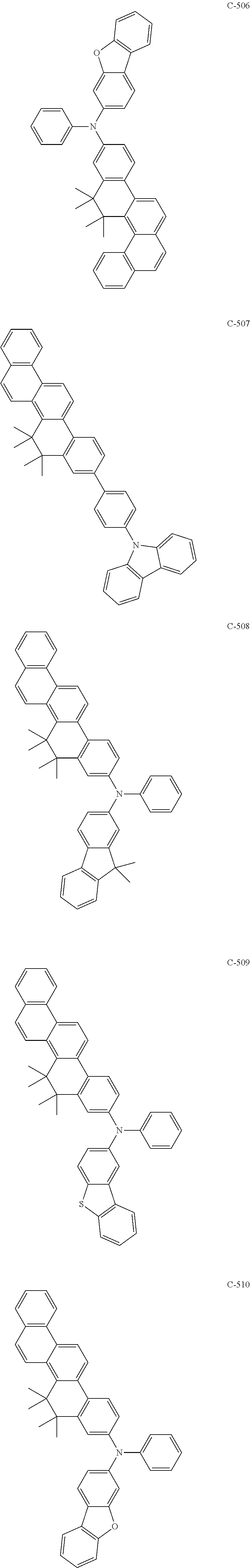 [SMILES: CC1(C)c2cc(N(c3ccccc3)c3ccc4c(c3)oc3ccccc34)ccc2-c2ccc3ccc4ccccc4c3c2C1(C)C ; CC1(C)c2cc(-c3ccc(-n4c5ccccc5c5ccccc54)cc3)ccc2-c2ccc3c(ccc4ccccc43)c2C1(C)C ; CC1(C)c2ccccc2-c2ccc(N(c3ccccc3)c3ccc4c(c3)C(C)(C)C(C)(C)c3c-4ccc4c3ccc3ccccc34)cc21 ; CC1(C)c2cc(N(c3ccccc3)c3ccc4sc5ccccc5c4c3)ccc2-c2ccc3c(ccc4ccccc43)c2C1(C)C ; CC1(C)c2cc(N(c3ccccc3)c3ccc4c(c3)oc3ccccc34)ccc2-c2ccc3c(ccc4ccccc43)c2C1(C)C]

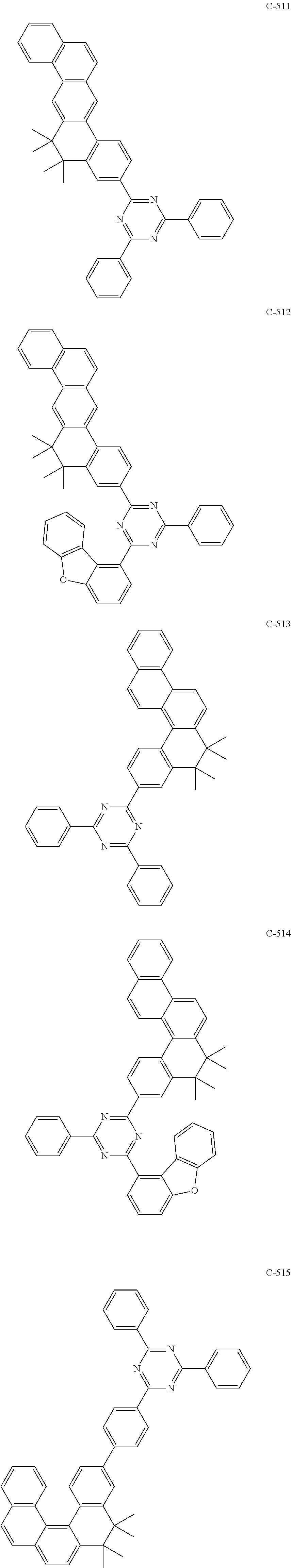 [SMILES: CC1(C)c2cc(-c3nc(-c4ccccc4)nc(-c4ccccc4)n3)ccc2-c2cc3ccc4ccccc4c3cc2C1(C)C ; CC1(C)c2cc(-c3nc(-c4ccccc4)nc(-c4cccc5oc6ccccc6c45)n3)ccc2-c2cc3ccc4ccccc4c3cc2C1(C)C ; CC1(C)c2cc(-c3nc(-c4ccccc4)nc(-c4ccccc4)n3)ccc2-c2c(ccc3c2ccc2ccccc23)C1(C)C ; CC1(C)c2cc(-c3nc(-c4ccccc4)nc(-c4cccc5oc6ccccc6c45)n3)ccc2-c2c(ccc3c2ccc2ccccc23)C1(C)C ; CC1(C)c2cc(-c3ccc(-c4nc(-c5ccccc5)nc(-c5ccccc5)n4)cc3)ccc2-c2c(ccc3ccc4ccccc4c23)C1(C)C]

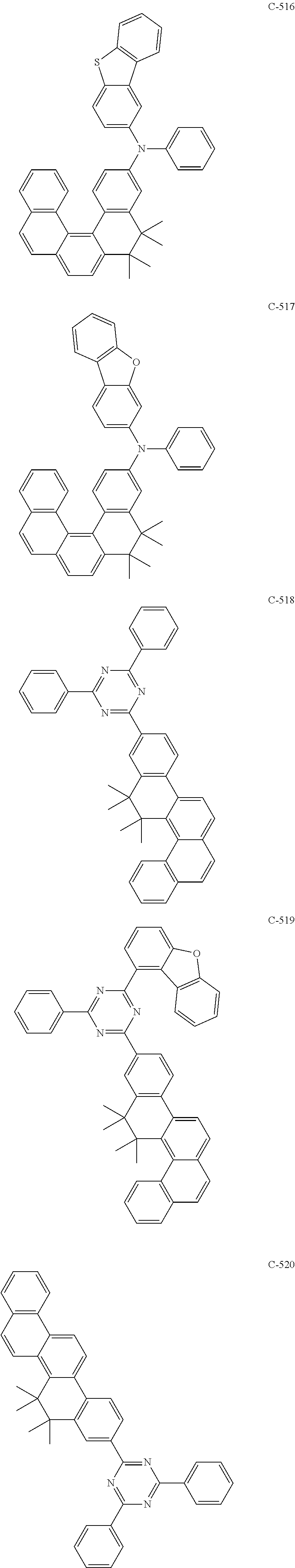

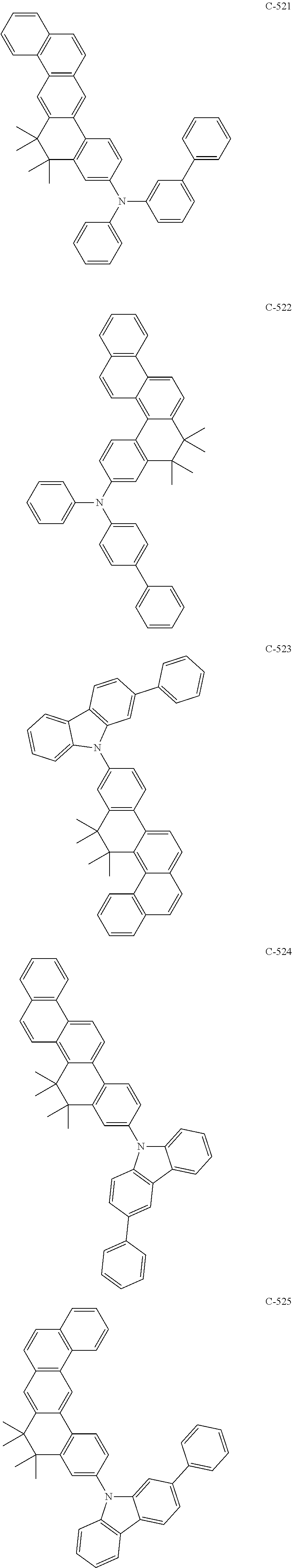

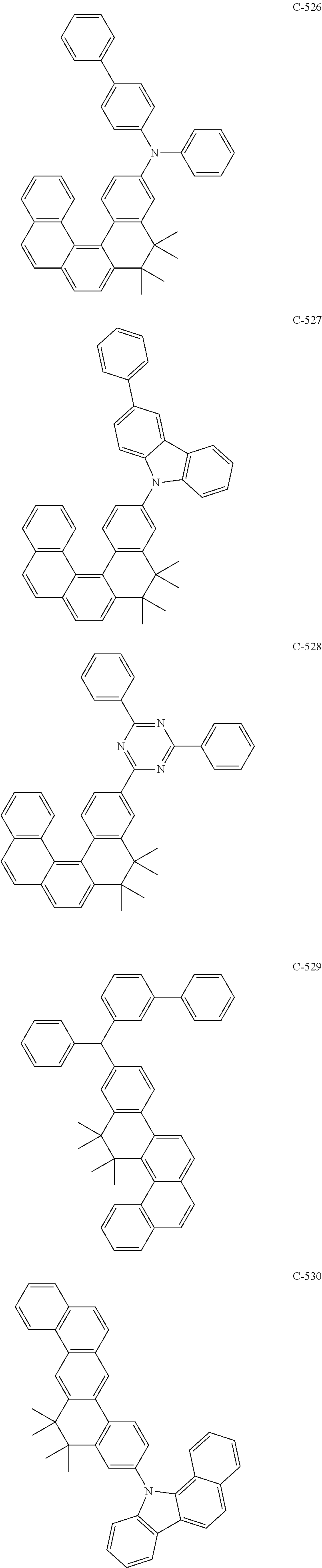

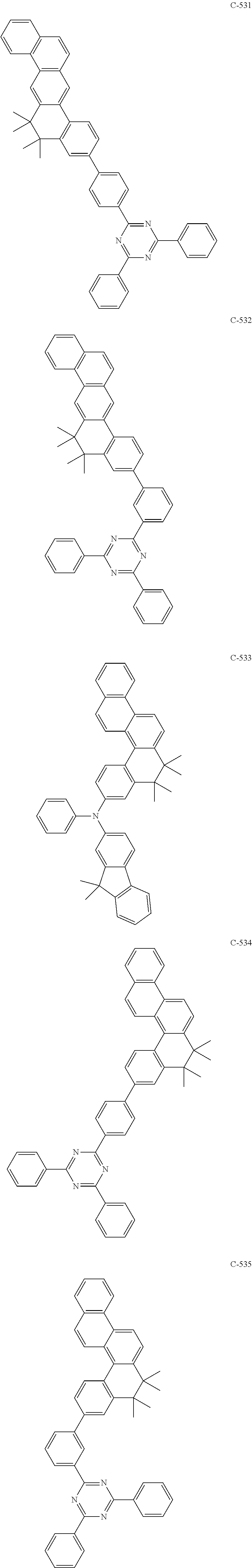

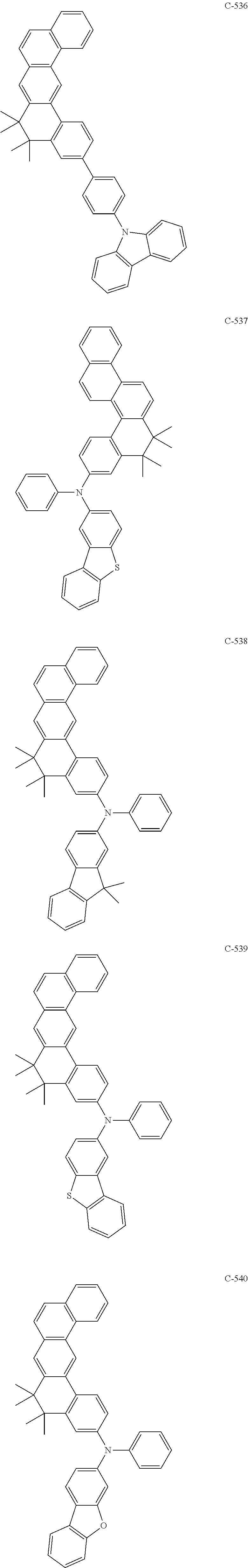

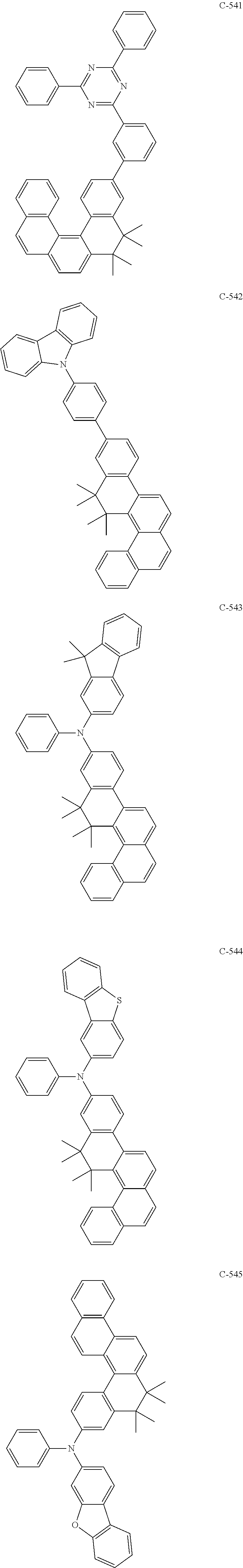

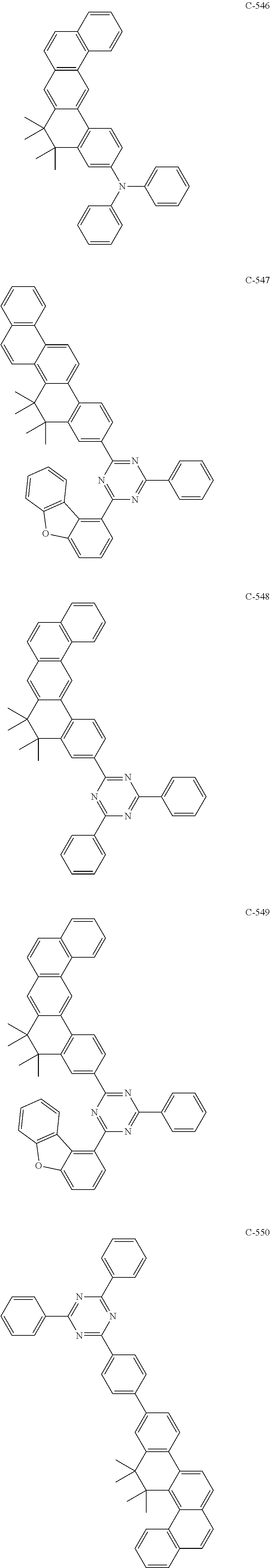 [SMILES: CC1(C)c2cc(N(c3ccccc3)c3ccccc3)ccc2-c2cc3c(ccc4ccccc43)cc2C1(C)C ; CC1(C)c2cc(-c3nc(-c4ccccc4)nc(-c4cccc5oc6ccccc6c45)n3)ccc2-c2ccc3c(ccc4ccccc43)c2C1(C)C ; CC1(C)c2cc(-c3nc(-c4ccccc4)nc(-c4ccccc4)n3)ccc2-c2cc3c(ccc4ccccc43)cc2C1(C)C ; CC1(C)c2cc(-c3nc(-c4ccccc4)nc(-c4cccc5oc6ccccc6c45)n3)ccc2-c2cc3c(ccc4ccccc43)cc2C1(C)C ; CC1(C)c2cc(-c3ccc(-c4nc(-c5ccccc5)nc(-c5ccccc5)n4)cc3)ccc2-c2ccc3ccc4ccccc4c3c2C1(C)C]

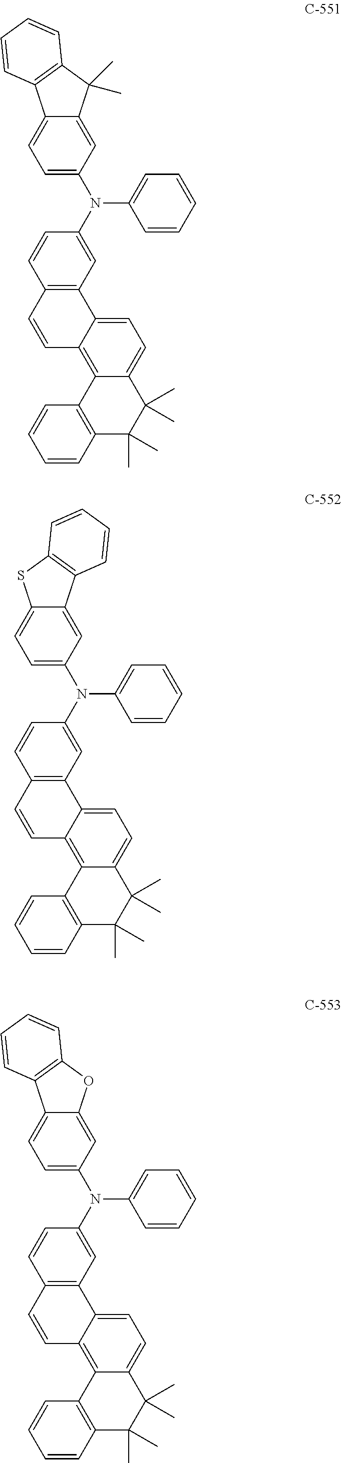

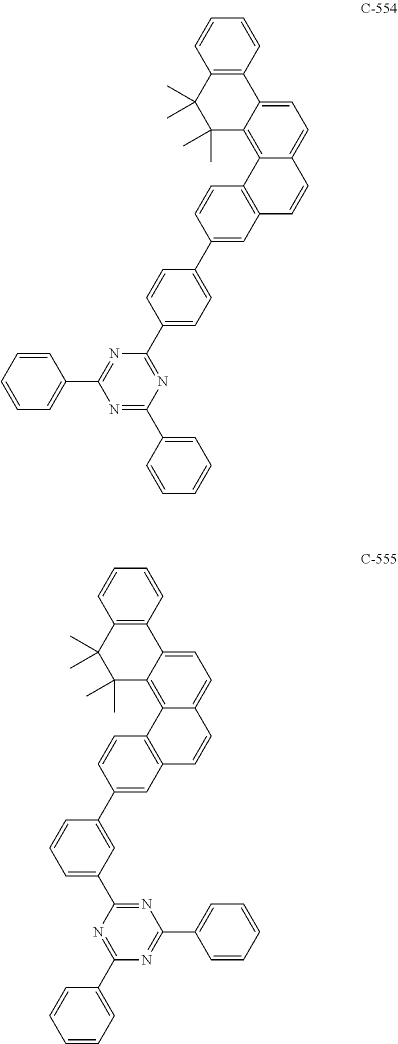 [SMILES: CC1(C)c2ccccc2-c2ccc3ccc4cc(-c5ccc(-c6nc(-c7ccccc7)nc(-c7ccccc7)n6)cc5)ccc4c3c2C1(C)C ; CC1(C)c2ccccc2-c2ccc3ccc4cc(-c5cccc(-c6nc(-c7ccccc7)nc(-c7ccccc7)n6)c5)ccc4c3c2C1(C)C]

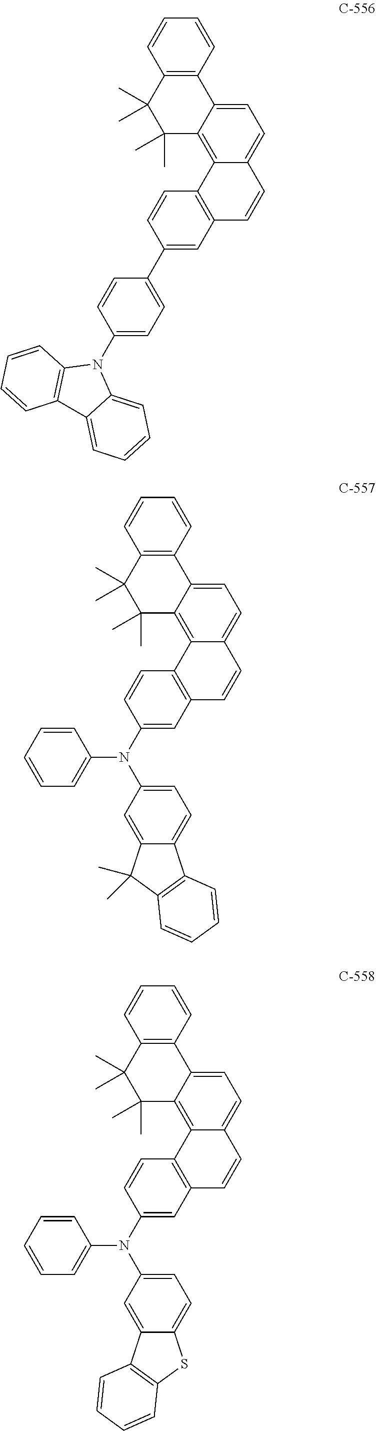

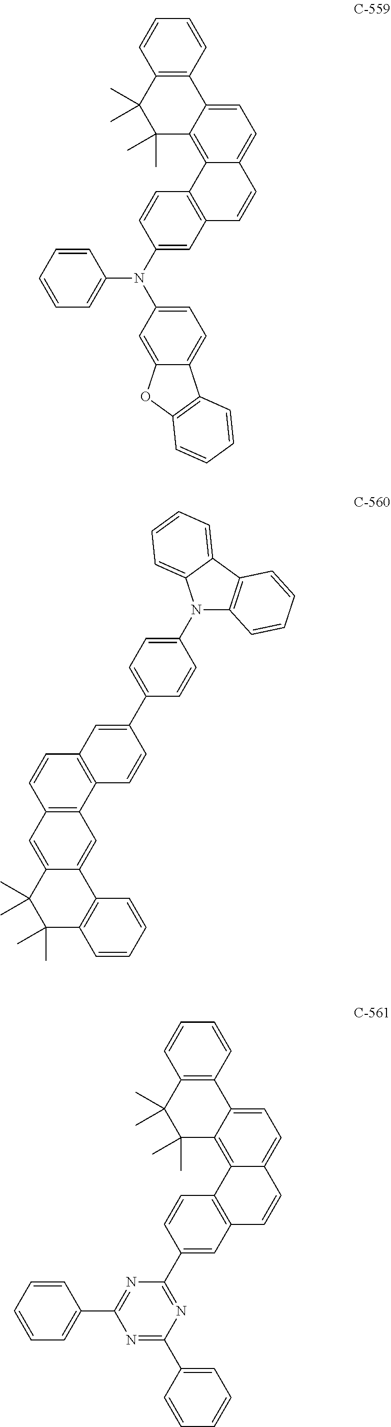 [SMILES: CC1(C)c2ccccc2-c2ccc3ccc4cc(N(c5ccccc5)c5ccc6c(c5)oc5ccccc56)ccc4c3c2C1(C)C ; CC1(C)c2ccccc2-c2cc3c(ccc4cc(-c5ccc(-n6c7ccccc7c7ccccc76)cc5)ccc43)cc2C1(C)C ; CC1(C)c2ccccc2-c2ccc3ccc4cc(-c5nc(-c6ccccc6)nc(-c6ccccc6)n5)ccc4c3c2C1(C)C]

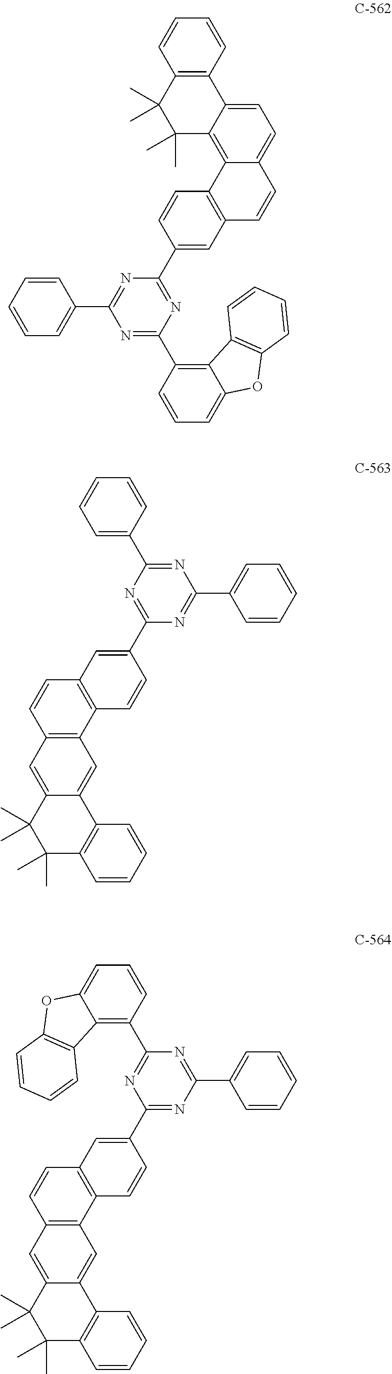 [SMILES: CC1(C)c2ccccc2-c2ccc3ccc4cc(-c5nc(-c6ccccc6)nc(-c6cccc7oc8ccccc8c67)n5)ccc4c3c2C1(C)C ; CC1(C)c2ccccc2-c2cc3c(ccc4cc(-c5nc(-c6ccccc6)nc(-c6ccccc6)n5)ccc43)cc2C1(C)C ; CC1(C)c2ccccc2-c2cc3c(ccc4cc(-c5nc(-c6ccccc6)nc(-c6cccc7oc8ccccc8c67)n5)ccc43)cc2C1(C)C]

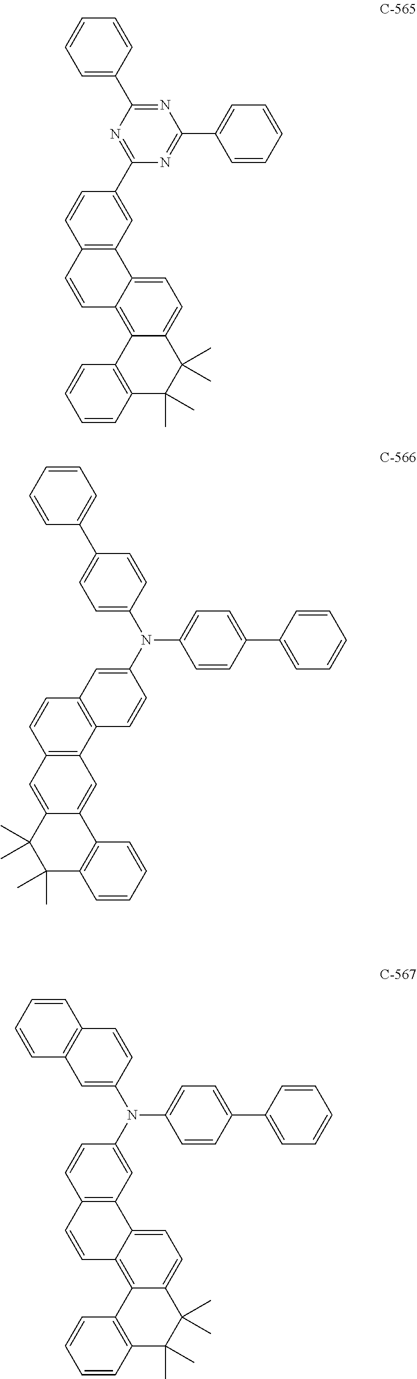

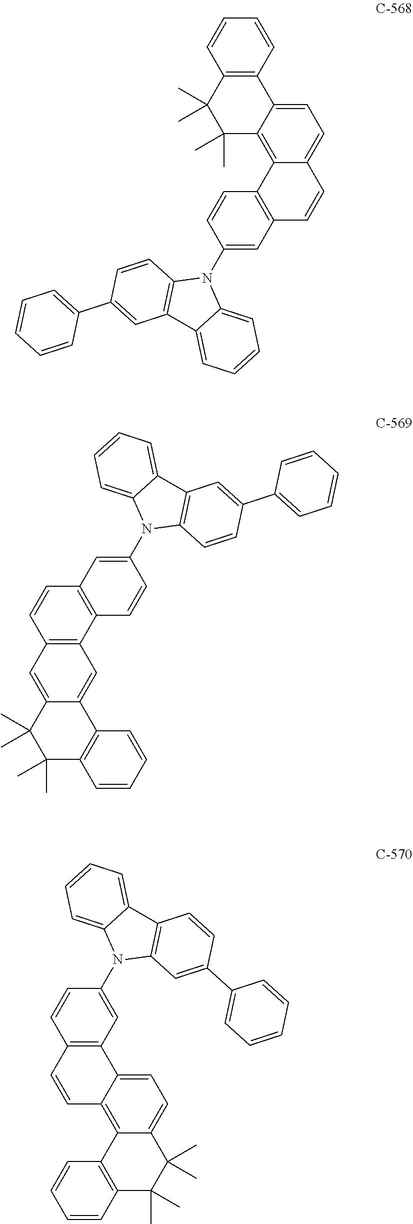 [SMILES: CC1(C)c2ccccc2-c2ccc3ccc4cc(-n5c6ccccc6c6cc(-c7ccccc7)ccc65)ccc4c3c2C1(C)C ; CC1(C)c2ccccc2-c2cc3c(ccc4cc(-n5c6ccccc6c6cc(-c7ccccc7)ccc65)ccc43)cc2C1(C)C ; CC1(C)c2ccccc2-c2c(ccc3c2ccc2ccc(-n4c5ccccc5c5ccc(-c6ccccc6)cc54)cc23)C1(C)C]

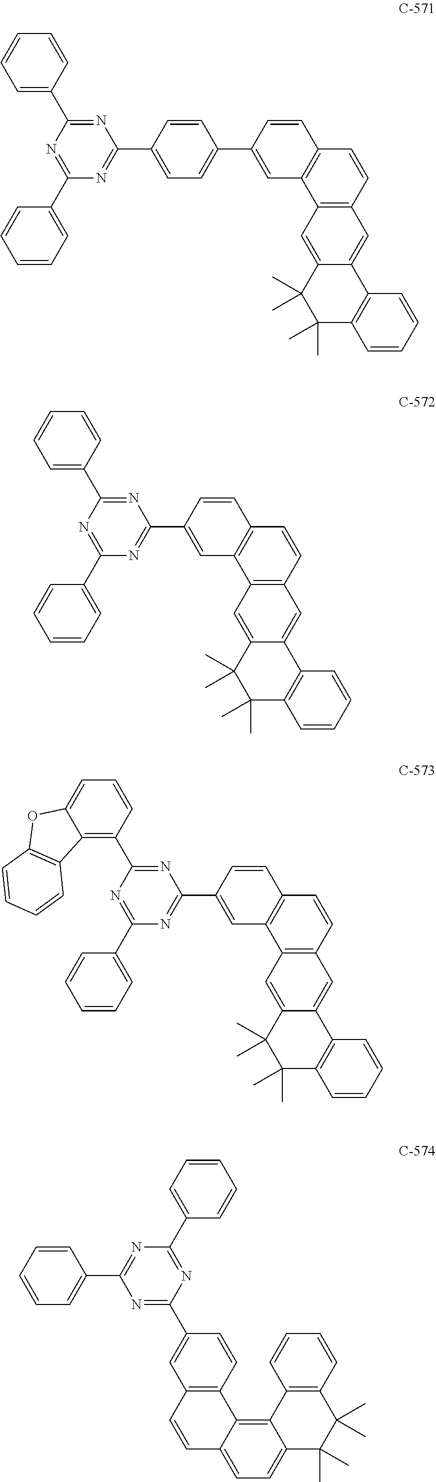 [SMILES: CC1(C)c2ccccc2-c2cc3ccc4ccc(-c5ccc(-c6nc(-c7ccccc7)nc(-c7ccccc7)n6)cc5)cc4c3cc2C1(C)C ; CC1(C)c2ccccc2-c2cc3ccc4ccc(-c5nc(-c6ccccc6)nc(-c6ccccc6)n5)cc4c3cc2C1(C)C ; CC1(C)c2ccccc2-c2cc3ccc4ccc(-c5nc(-c6ccccc6)nc(-c6cccc7oc8ccccc8c67)n5)cc4c3cc2C1(C)C ; CC1(C)c2ccccc2-c2c(ccc3ccc4cc(-c5nc(-c6ccccc6)nc(-c6ccccc6)n5)ccc4c23)C1(C)C]

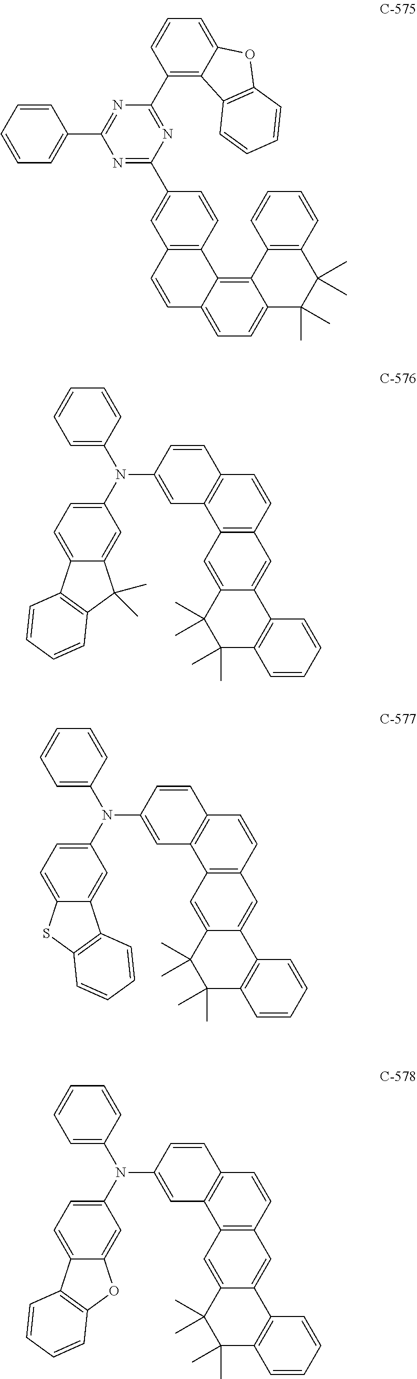 [SMILES: CC1(C)c2ccccc2-c2c(ccc3ccc4cc(-c5nc(-c6ccccc6)nc(-c6cccc7oc8ccccc8c67)n5)ccc4c23)C1(C)C ; CC1(C)c2ccccc2-c2ccc(N(c3ccccc3)c3ccc4ccc5cc6c(cc5c4c3)C(C)(C)C(C)(C)c3ccccc3-6)cc21 ; CC1(C)c2ccccc2-c2cc3ccc4ccc(N(c5ccccc5)c5ccc6sc7ccccc7c6c5)cc4c3cc2C1(C)C ; CC1(C)c2ccccc2-c2cc3ccc4ccc(N(c5ccccc5)c5ccc6c(c5)oc5ccccc56)cc4c3cc2C1(C)C]

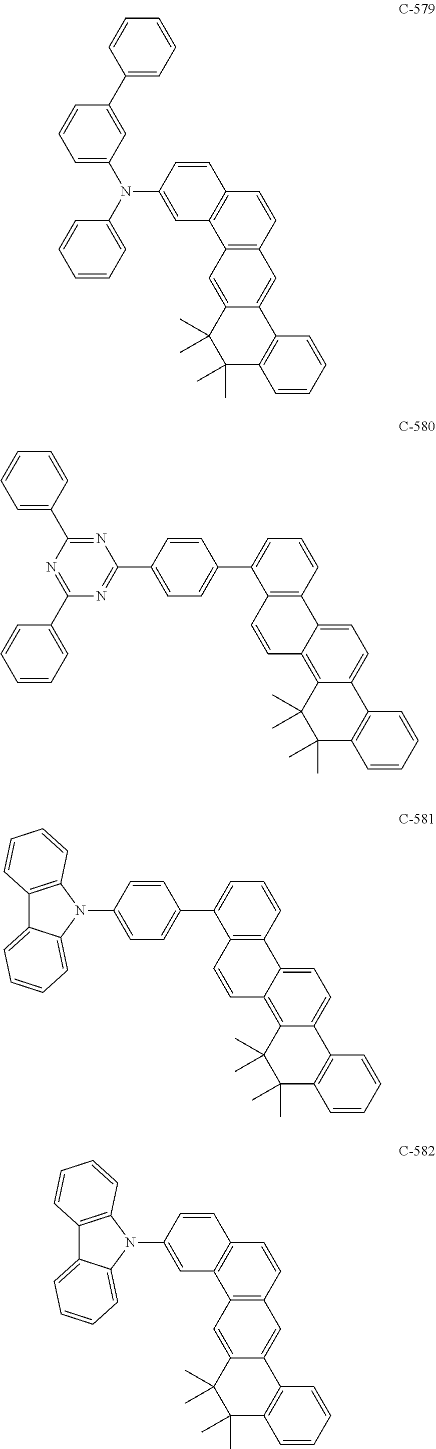 [SMILES: CC1(C)c2ccccc2-c2cc3ccc4ccc(N(c5ccccc5)c5cccc(-c6ccccc6)c5)cc4c3cc2C1(C)C ; CC1(C)c2ccccc2-c2ccc3c(ccc4c(-c5ccc(-c6nc(-c7ccccc7)nc(-c7ccccc7)n6)cc5)cccc43)c2C1(C)C ; CC1(C)c2ccccc2-c2ccc3c(ccc4c(-c5ccc(-n6c7ccccc7c7ccccc76)cc5)cccc43)c2C1(C)C ; CC1(C)c2ccccc2-c2cc3ccc4ccc(-n5c6ccccc6c6ccccc65)cc4c3cc2C1(C)C]

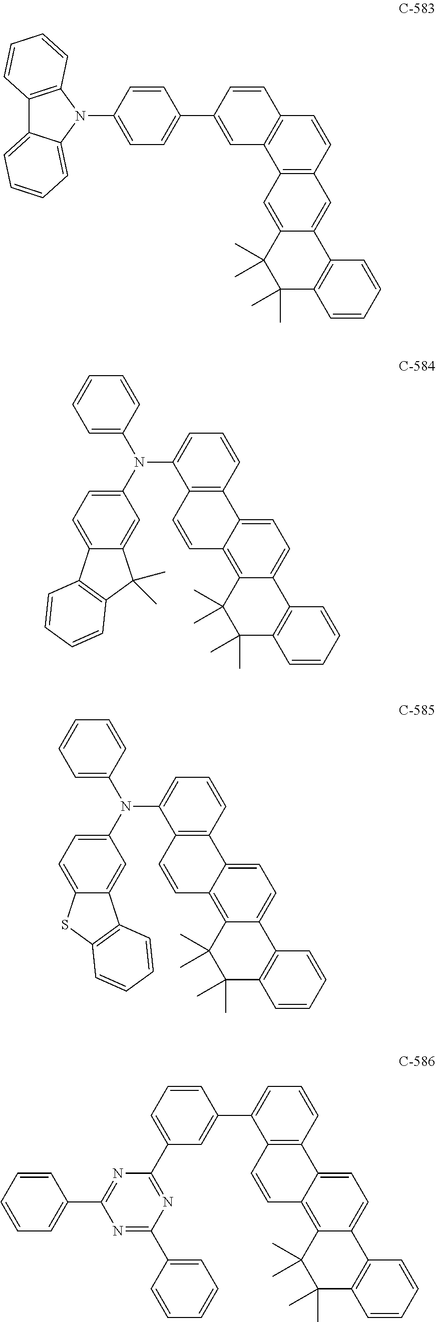 [SMILES: CC1(C)c2ccccc2-c2cc3ccc4ccc(-c5ccc(-n6c7ccccc7c7ccccc76)cc5)cc4c3cc2C1(C)C ; CC1(C)c2ccccc2-c2ccc(N(c3ccccc3)c3cccc4c3ccc3c5c(ccc34)-c3ccccc3C(C)(C)C5(C)C)cc21 ; CC1(C)c2ccccc2-c2ccc3c(ccc4c(N(c5ccccc5)c5ccc6sc7ccccc7c6c5)cccc43)c2C1(C)C ; CC1(C)c2ccccc2-c2ccc3c(ccc4c(-c5cccc(-c6nc(-c7ccccc7)nc(-c7ccccc7)n6)c5)cccc43)c2C1(C)C]

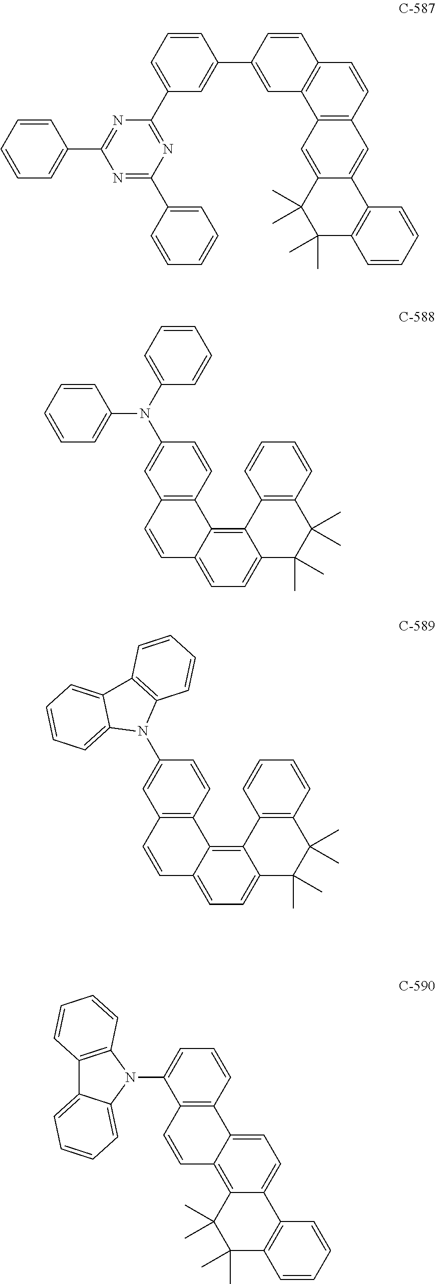 [SMILES: CC1(C)c2ccccc2-c2cc3ccc4ccc(-c5cccc(-c6nc(-c7ccccc7)nc(-c7ccccc7)n6)c5)cc4c3cc2C1(C)C ; CC1(C)c2ccccc2-c2c(ccc3ccc4cc(N(c5ccccc5)c5ccccc5)ccc4c23)C1(C)C ; CC1(C)c2ccccc2-c2c(ccc3ccc4cc(-n5c6ccccc6c6ccccc65)ccc4c23)C1(C)C ; CC1(C)c2ccccc2-c2ccc3c(ccc4c(-n5c6ccccc6c6ccccc65)cccc43)c2C1(C)C]

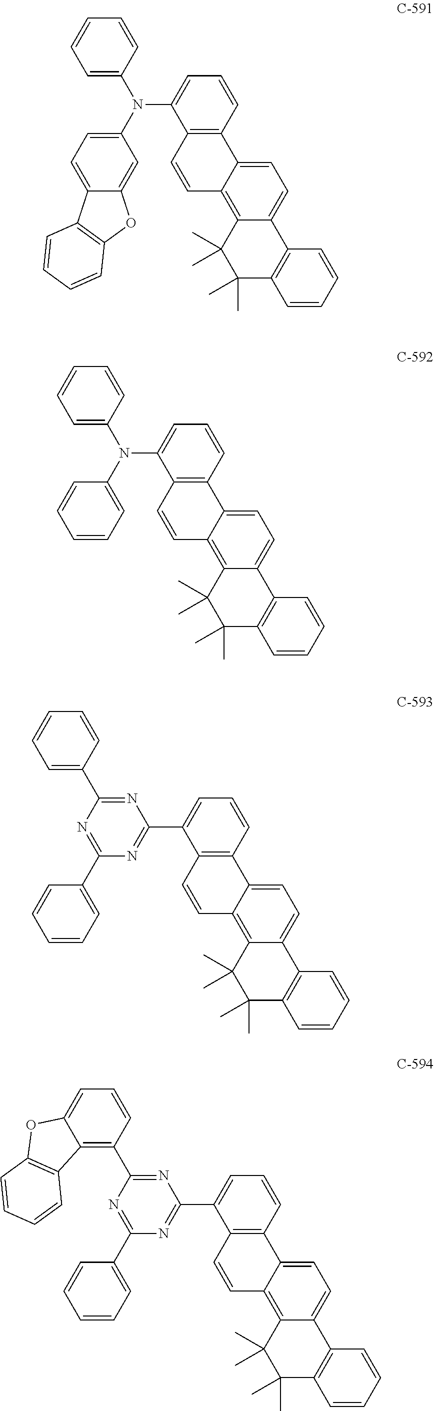 [SMILES: CC1(C)c2ccccc2-c2ccc3c(ccc4c(N(c5ccccc5)c5ccc6c(c5)oc5ccccc56)cccc43)c2C1(C)C ; CC1(C)c2ccccc2-c2ccc3c(ccc4c(N(c5ccccc5)c5ccccc5)cccc43)c2C1(C)C ; CC1(C)c2ccccc2-c2ccc3c(ccc4c(-c5nc(-c6ccccc6)nc(-c6ccccc6)n5)cccc43)c2C1(C)C ; CC1(C)c2ccccc2-c2ccc3c(ccc4c(-c5nc(-c6ccccc6)nc(-c6cccc7oc8ccccc8c67)n5)cccc43)c2C1(C)C]

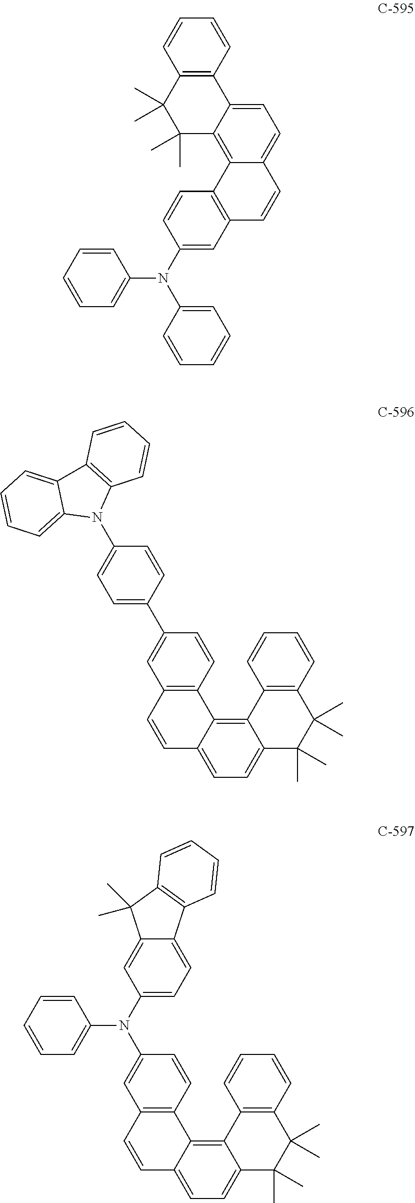 [SMILES: CC1(C)c2ccccc2-c2ccc3ccc4cc(N(c5ccccc5)c5ccccc5)ccc4c3c2C1(C)C ; CC1(C)c2ccccc2-c2c(ccc3ccc4cc(-c5ccc(-n6c7ccccc7c7ccccc76)cc5)ccc4c23)C1(C)C ; CC1(C)c2ccccc2-c2ccc(N(c3ccccc3)c3ccc4c(ccc5ccc6c(c54)-c4ccccc4C(C)(C)C6(C)C)c3)cc21]

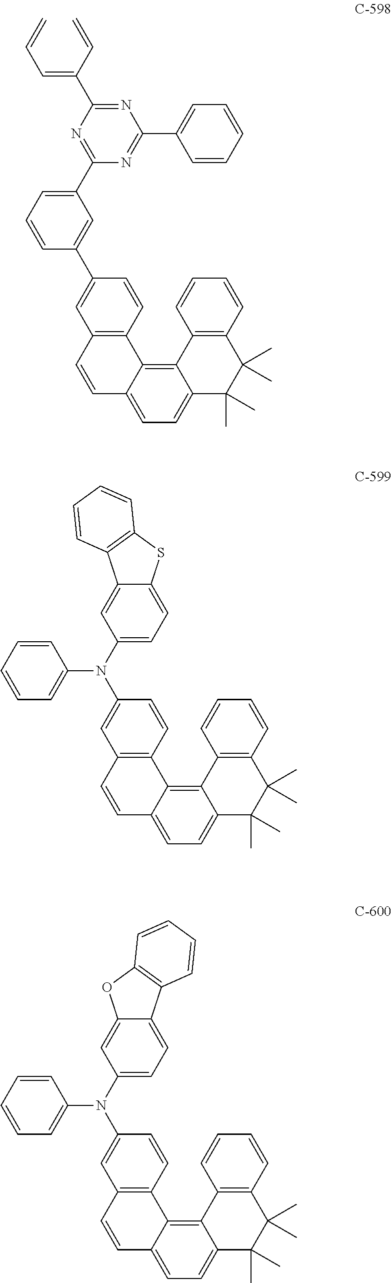 [SMILES: C=C/C=C(\C=C)c1nc(-c2ccccc2)nc(-c2cccc(-c3ccc4c(ccc5ccc6c(c54)-c4ccccc4C(C)(C)C6(C)C)c3)c2)n1 ; CC1(C)c2ccccc2-c2c(ccc3ccc4cc(N(c5ccccc5)c5ccc6sc7ccccc7c6c5)ccc4c23)C1(C)C ; CC1(C)c2ccccc2-c2c(ccc3ccc4cc(N(c5ccccc5)c5ccc6c(c5)oc5ccccc56)ccc4c23)C1(C)C]

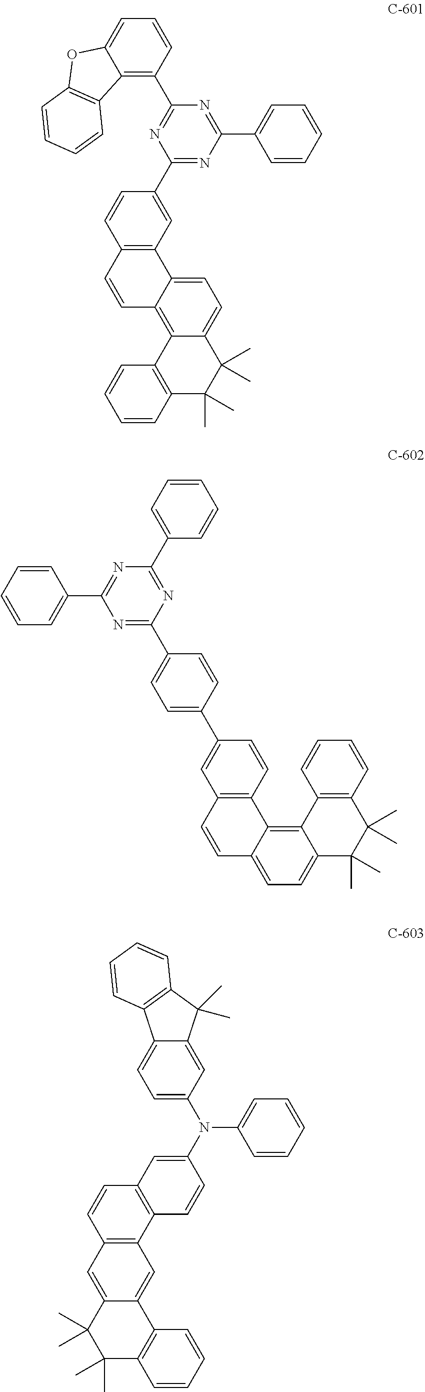

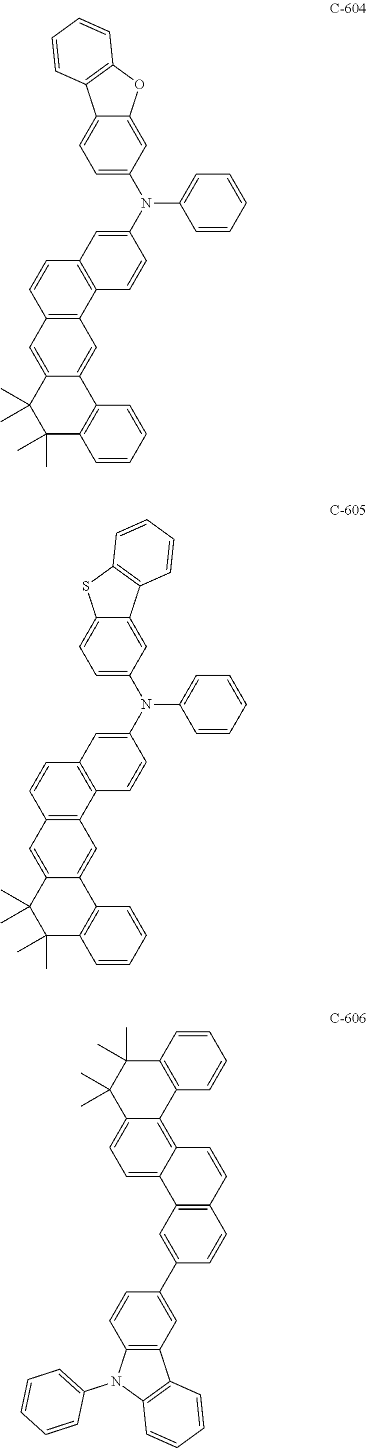 [SMILES: CC1(C)c2ccccc2-c2cc3c(ccc4cc(N(c5ccccc5)c5ccc6c(c5)oc5ccccc56)ccc43)cc2C1(C)C ; CC1(C)c2ccccc2-c2cc3c(ccc4cc(N(c5ccccc5)c5ccc6sc7ccccc7c6c5)ccc43)cc2C1(C)C ; CC1(C)c2ccccc2-c2c(ccc3c2ccc2ccc(-c4ccc5c(c4)c4ccccc4n5-c4ccccc4)cc23)C1(C)C]

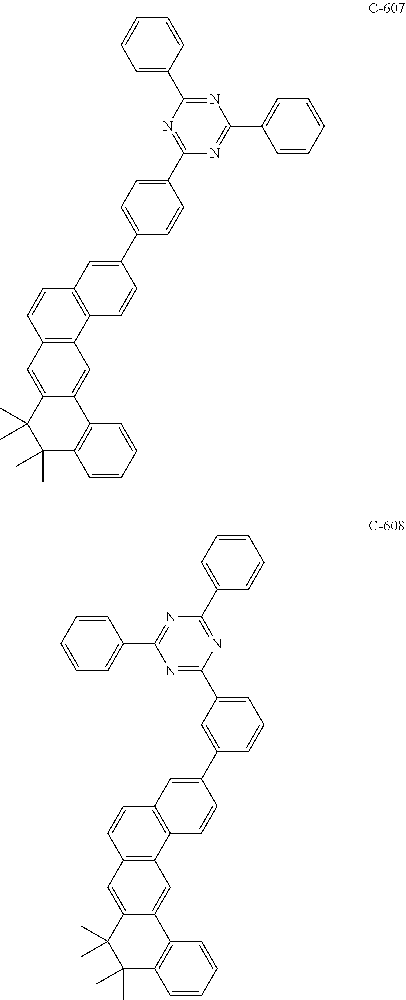 [SMILES: CC1(C)c2ccccc2-c2cc3c(ccc4cc(-c5ccc(-c6nc(-c7ccccc7)nc(-c7ccccc7)n6)cc5)ccc43)cc2C1(C)C ; CC1(C)c2ccccc2-c2cc3c(ccc4cc(-c5cccc(-c6nc(-c7ccccc7)nc(-c7ccccc7)n6)c5)ccc43)cc2C1(C)C]

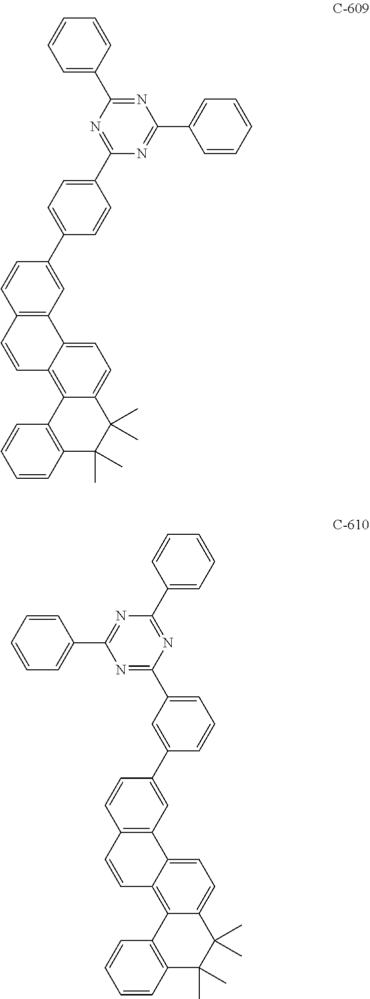 [SMILES: CC1(C)c2ccccc2-c2c(ccc3c2ccc2ccc(-c4ccc(-c5nc(-c6ccccc6)nc(-c6ccccc6)n5)cc4)cc23)C1(C)C ; CC1(C)c2ccccc2-c2c(ccc3c2ccc2ccc(-c4cccc(-c5nc(-c6ccccc6)nc(-c6ccccc6)n5)c4)cc23)C1(C)C]

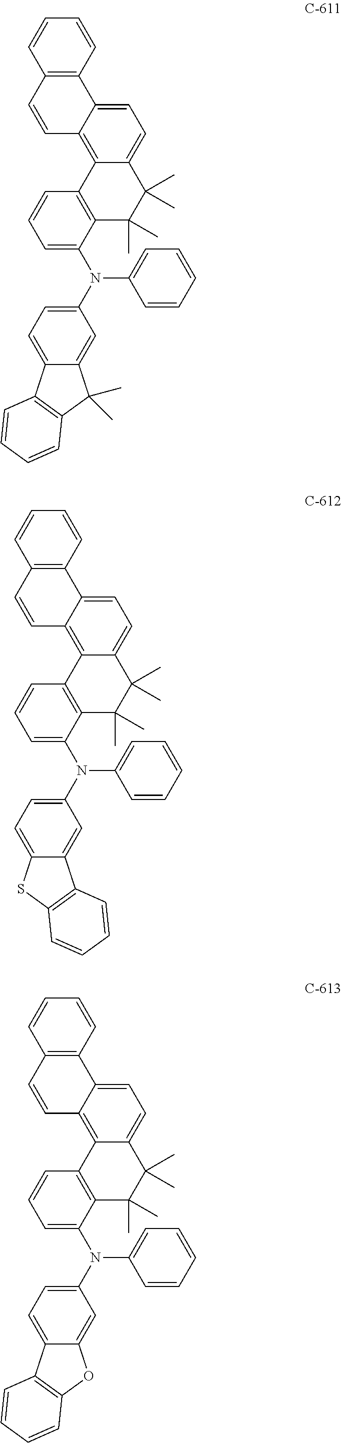

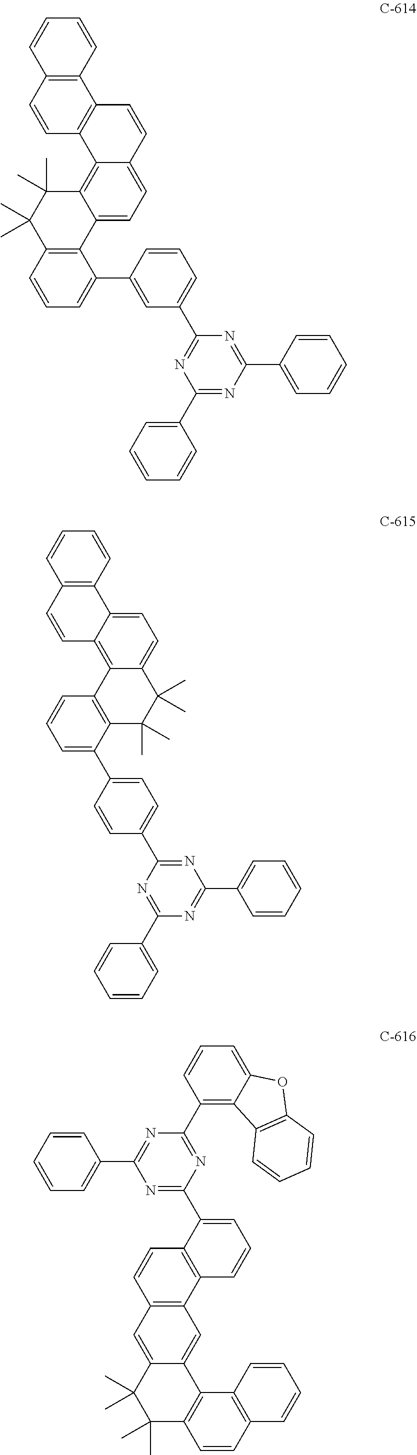 [SMILES: CC1(C)c2cccc(-c3cccc(-c4nc(-c5ccccc5)nc(-c5ccccc5)n4)c3)c2-c2ccc3ccc4c5ccccc5ccc4c3c2C1(C)C ; CC1(C)c2ccc3c(ccc4ccccc43)c2-c2cccc(-c3ccc(-c4nc(-c5ccccc5)nc(-c5ccccc5)n4)cc3)c2C1(C)C ; CC1(C)c2cc3ccc4c(-c5nc(-c6ccccc6)nc(-c6cccc7oc8ccccc8c67)n5)cccc4c3cc2-c2c(ccc3ccccc23)C1(C)C]

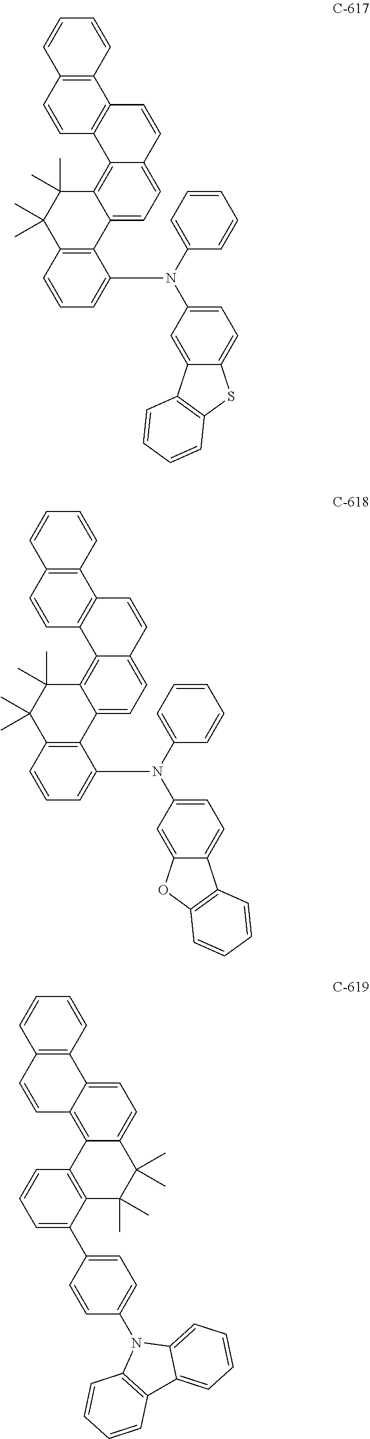 [SMILES: CC1(C)c2cccc(N(c3ccccc3)c3ccc4sc5ccccc5c4c3)c2-c2ccc3ccc4c5ccccc5ccc4c3c2C1(C)C ; CC1(C)c2cccc(N(c3ccccc3)c3ccc4c(c3)oc3ccccc34)c2-c2ccc3ccc4c5ccccc5ccc4c3c2C1(C)C ; CC1(C)c2ccc3c(ccc4ccccc43)c2-c2cccc(-c3ccc(-n4c5ccccc5c5ccccc54)cc3)c2C1(C)C]

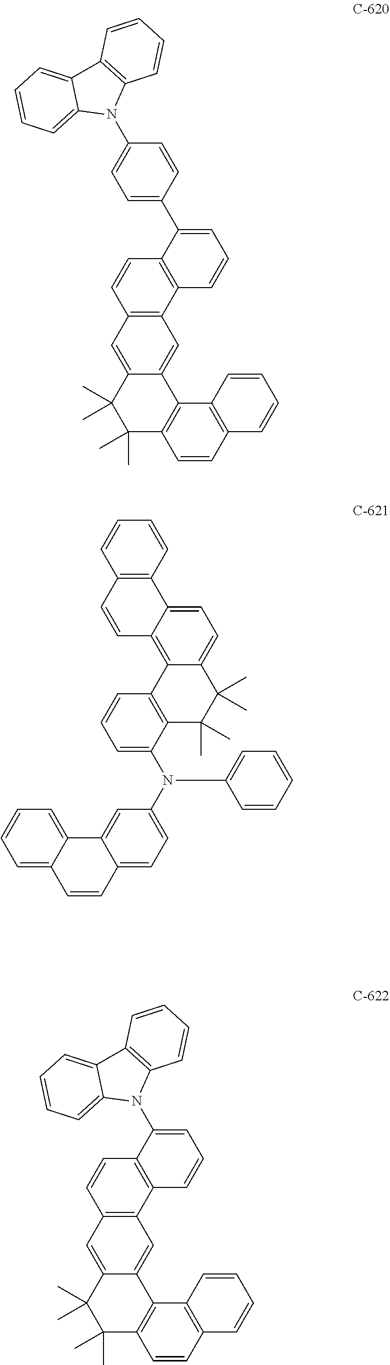 [SMILES: CC1(C)c2cc3ccc4c(-c5ccc(-n6c7ccccc7c7ccccc76)cc5)cccc4c3cc2-c2c(ccc3ccccc23)C1(C)C ; CC1(C)c2ccc3c(ccc4ccccc43)c2-c2cccc(N(c3ccccc3)c3ccc4ccc5ccccc5c4c3)c2C1(C)C ; CC1(C)c2cc3ccc4c(-n5c6ccccc6c6ccccc65)cccc4c3cc2-c2c(ccc3ccccc23)C1(C)C]

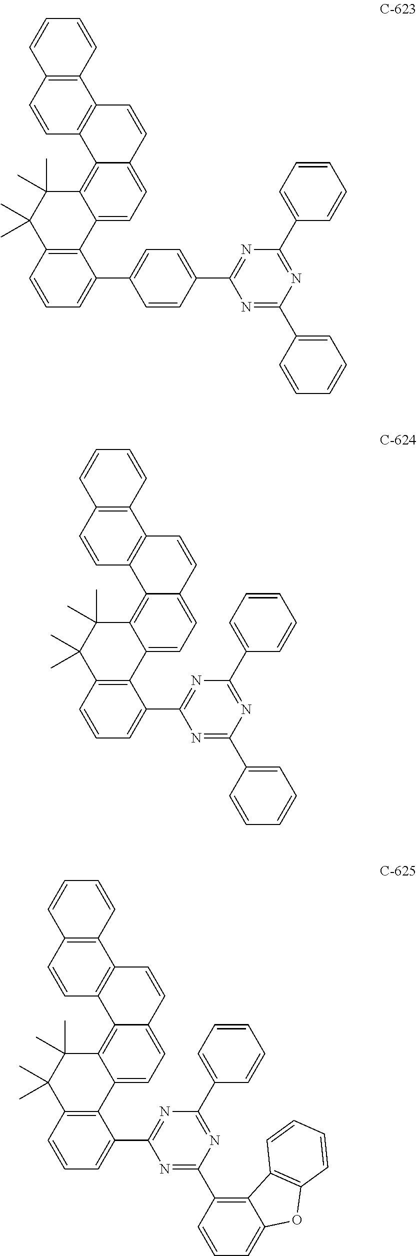 [SMILES: CC1(C)c2cccc(-c3ccc(-c4nc(-c5ccccc5)nc(-c5ccccc5)n4)cc3)c2-c2ccc3ccc4c5ccccc5ccc4c3c2C1(C)C ; CC1(C)c2cccc(-c3nc(-c4ccccc4)nc(-c4ccccc4)n3)c2-c2ccc3ccc4c5ccccc5ccc4c3c2C1(C)C ; CC1(C)c2cccc(-c3nc(-c4ccccc4)nc(-c4cccc5oc6ccccc6c45)n3)c2-c2ccc3ccc4c5ccccc5ccc4c3c2C1(C)C]

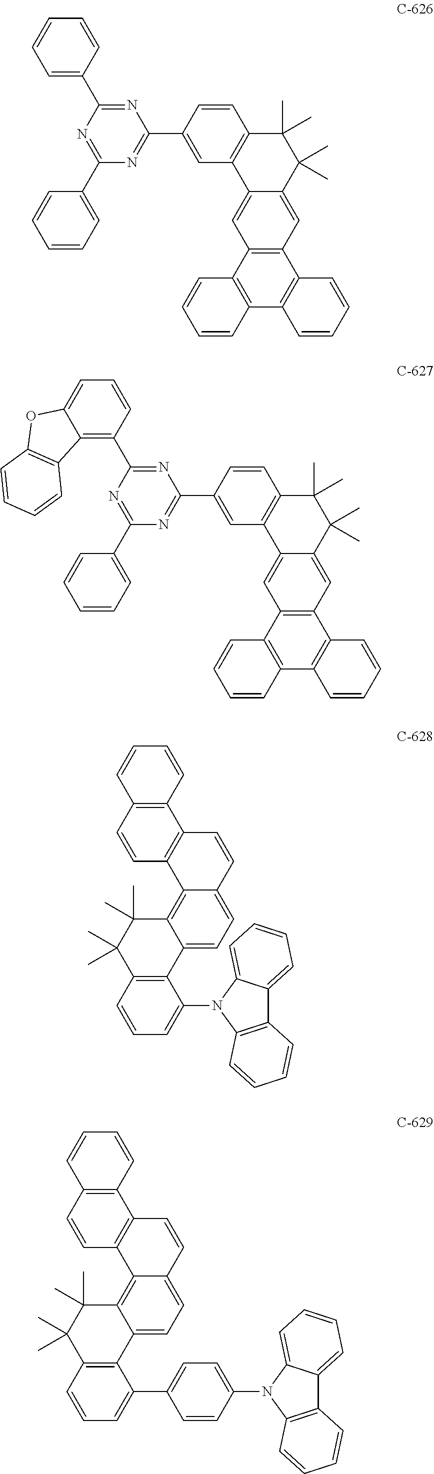 [SMILES: CC1(C)c2ccc(-c3nc(-c4ccccc4)nc(-c4ccccc4)n3)cc2-c2cc3c4ccccc4c4ccccc4c3cc2C1(C)C ; CC1(C)c2ccc(-c3nc(-c4ccccc4)nc(-c4cccc5oc6ccccc6c45)n3)cc2-c2cc3c4ccccc4c4ccccc4c3cc2C1(C)C ; CC1(C)c2cccc(-n3c4ccccc4c4ccccc43)c2-c2ccc3ccc4c5ccccc5ccc4c3c2C1(C)C ; CC1(C)c2cccc(-c3ccc(-n4c5ccccc5c5ccccc54)cc3)c2-c2ccc3ccc4c5ccccc5ccc4c3c2C1(C)C]

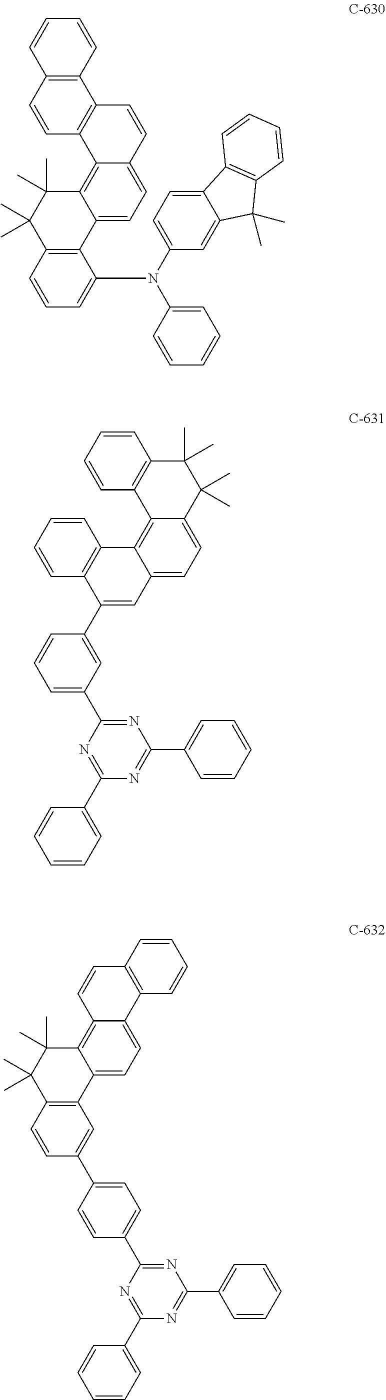

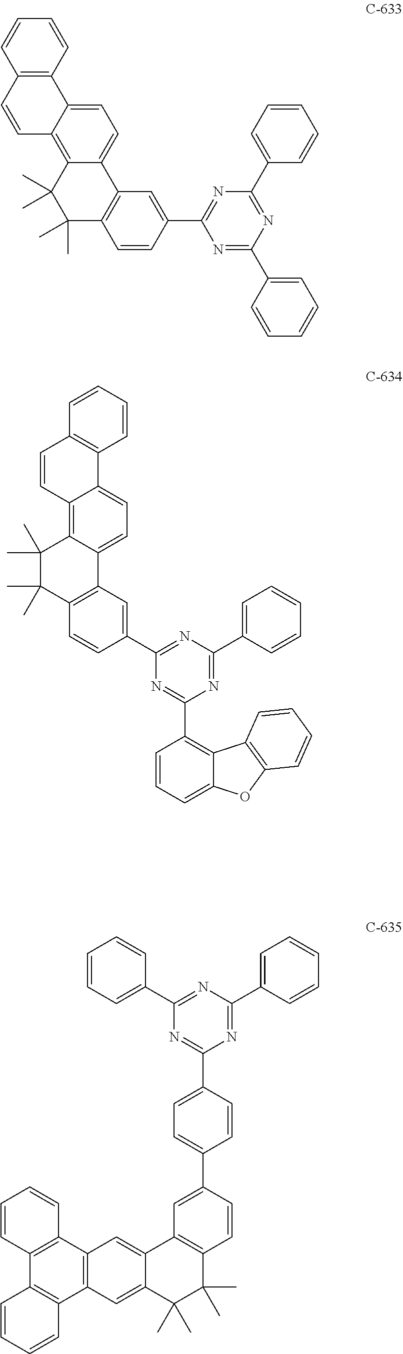 [SMILES: CC1(C)c2ccc(-c3nc(-c4ccccc4)nc(-c4ccccc4)n3)cc2-c2ccc3c(ccc4ccccc43)c2C1(C)C ; CC1(C)c2ccc(-c3nc(-c4ccccc4)nc(-c4cccc5oc6ccccc6c45)n3)cc2-c2ccc3c(ccc4ccccc43)c2C1(C)C ; CC1(C)c2ccc(-c3ccc(-c4nc(-c5ccccc5)nc(-c5ccccc5)n4)cc3)cc2-c2cc3c4ccccc4c4ccccc4c3cc2C1(C)C]

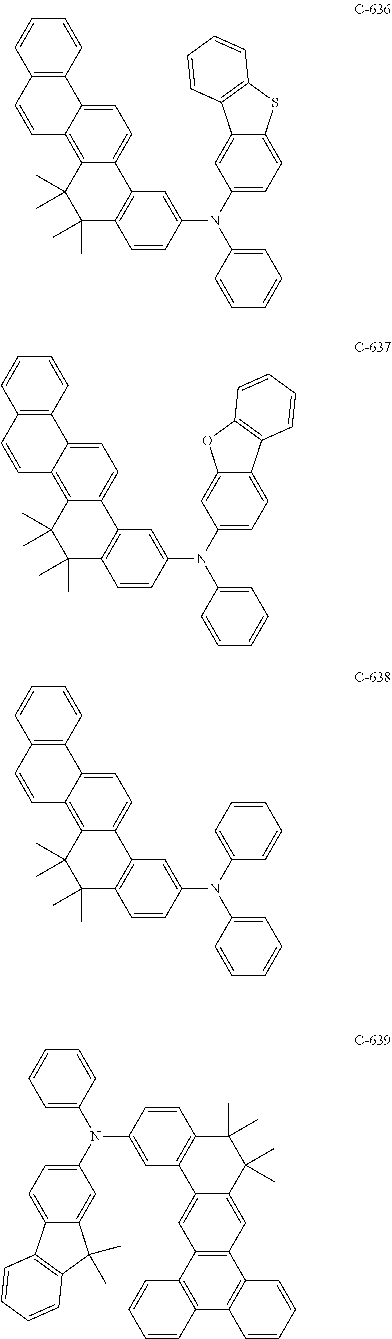 [SMILES: CC1(C)c2ccc(N(c3ccccc3)c3ccc4sc5ccccc5c4c3)cc2-c2ccc3c(ccc4ccccc43)c2C1(C)C ; CC1(C)c2ccc(N(c3ccccc3)c3ccc4c(c3)oc3ccccc34)cc2-c2ccc3c(ccc4ccccc43)c2C1(C)C ; CC1(C)c2ccc(N(c3ccccc3)c3ccccc3)cc2-c2ccc3c(ccc4ccccc43)c2C1(C)C ; CC1(C)c2ccccc2-c2ccc(N(c3ccccc3)c3ccc4c(c3)-c3cc5c6ccccc6c6ccccc6c5cc3C(C)(C)C4(C)C)cc21]

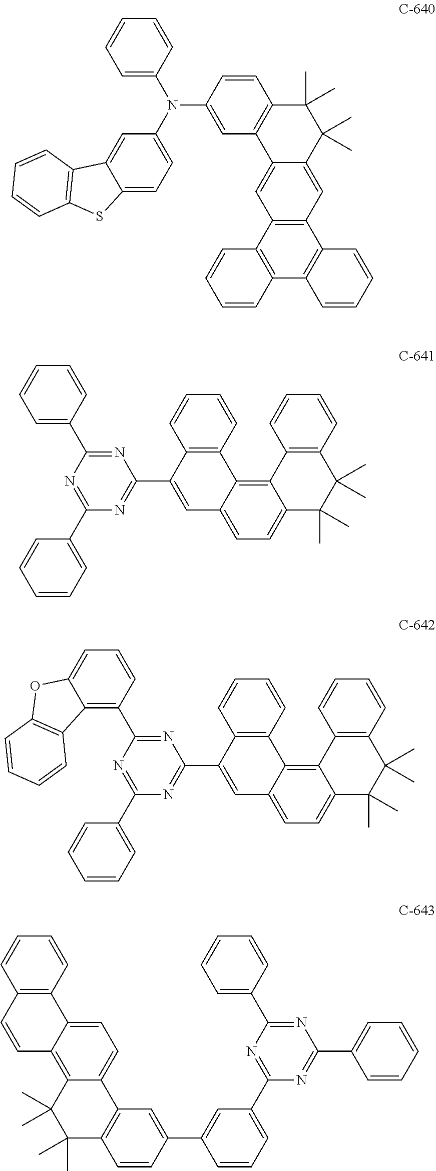 [SMILES: CC1(C)c2ccc(N(c3ccccc3)c3ccc4sc5ccccc5c4c3)cc2-c2cc3c4ccccc4c4ccccc4c3cc2C1(C)C ; CC1(C)c2ccccc2-c2c(ccc3cc(-c4nc(-c5ccccc5)nc(-c5ccccc5)n4)c4ccccc4c23)C1(C)C ; CC1(C)c2ccccc2-c2c(ccc3cc(-c4nc(-c5ccccc5)nc(-c5cccc6oc7ccccc7c56)n4)c4ccccc4c23)C1(C)C ; CC1(C)c2ccc(-c3cccc(-c4nc(-c5ccccc5)nc(-c5ccccc5)n4)c3)cc2-c2ccc3c(ccc4ccccc43)c2C1(C)C]

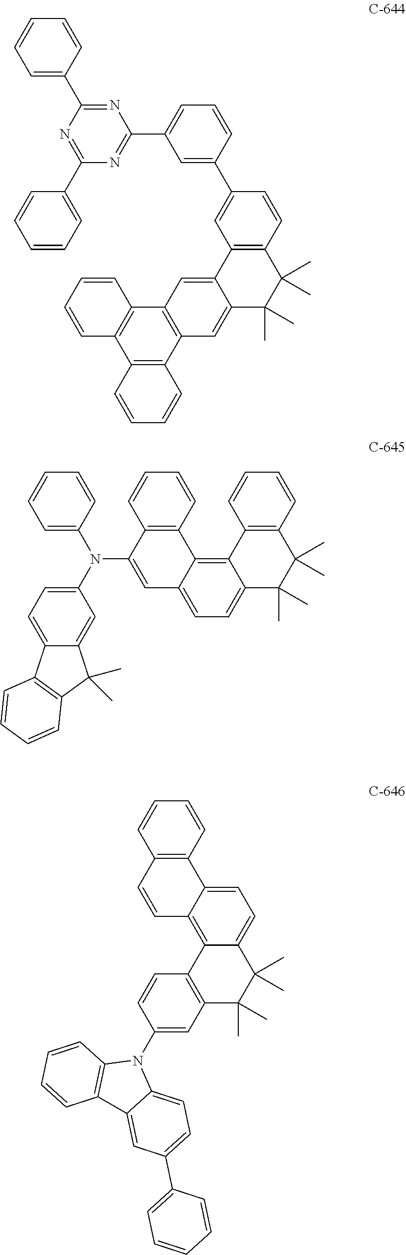 [SMILES: CC1(C)c2ccc(-c3cccc(-c4nc(-c5ccccc5)nc(-c5ccccc5)n4)c3)cc2-c2cc3c4ccccc4c4ccccc4c3cc2C1(C)C ; CC1(C)c2ccccc2-c2ccc(N(c3ccccc3)c3cc4ccc5c(c4c4ccccc34)-c3ccccc3C(C)(C)C5(C)C)cc21 ; CC1(C)c2cc(-n3c4ccccc4c4cc(-c5ccccc5)ccc43)ccc2-c2c(ccc3c2ccc2ccccc23)C1(C)C]

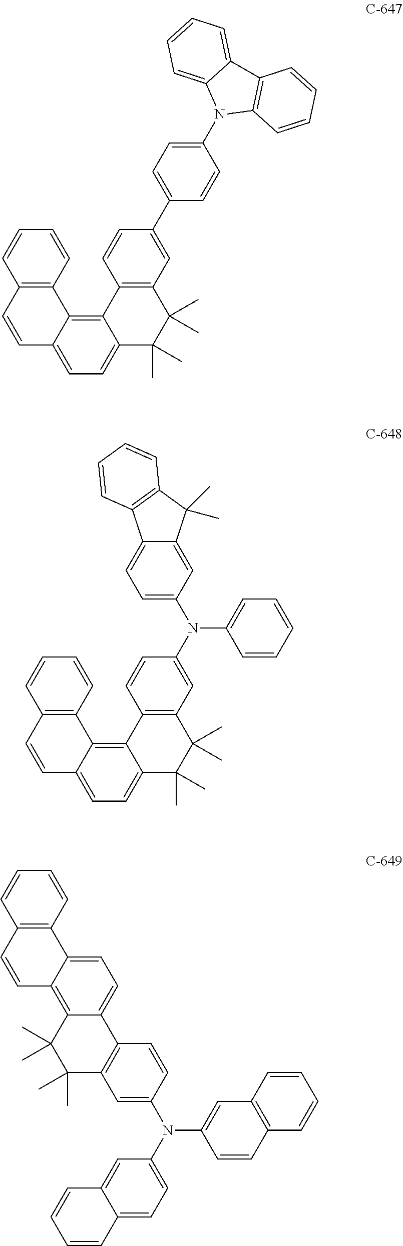 [SMILES: CC1(C)c2cc(-c3ccc(-n4c5ccccc5c5ccccc54)cc3)ccc2-c2c(ccc3ccc4ccccc4c23)C1(C)C ; CC1(C)c2ccccc2-c2ccc(N(c3ccccc3)c3ccc4c(c3)C(C)(C)C(C)(C)c3ccc5ccc6ccccc6c5c3-4)cc21 ; CC1(C)c2cc(N(c3ccc4ccccc4c3)c3ccc4ccccc4c3)ccc2-c2ccc3c(ccc4ccccc43)c2C1(C)C]

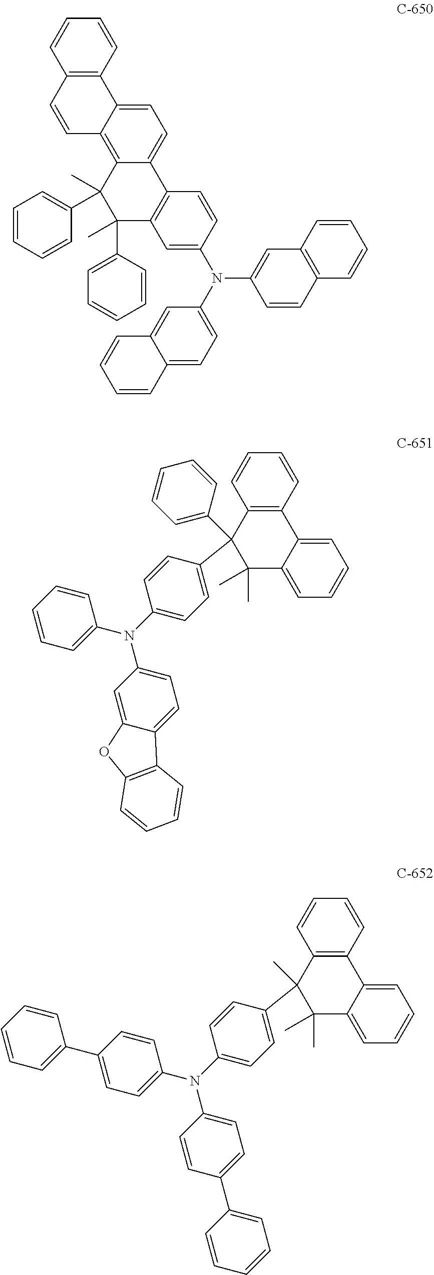 [SMILES: CC1(c2ccccc2)c2cc(N(c3ccc4ccccc4c3)c3ccc4ccccc4c3)ccc2-c2ccc3c(ccc4ccccc43)c2C1(C)c1ccccc1 ; CC1(C)c2ccccc2-c2ccccc2C1(c1ccccc1)c1ccc(N(c2ccccc2)c2ccc3c(c2)oc2ccccc23)cc1 ; CC1(C)c2ccccc2-c2ccccc2C1(C)c1ccc(N(c2ccc(-c3ccccc3)cc2)c2ccc(-c3ccccc3)cc2)cc1]

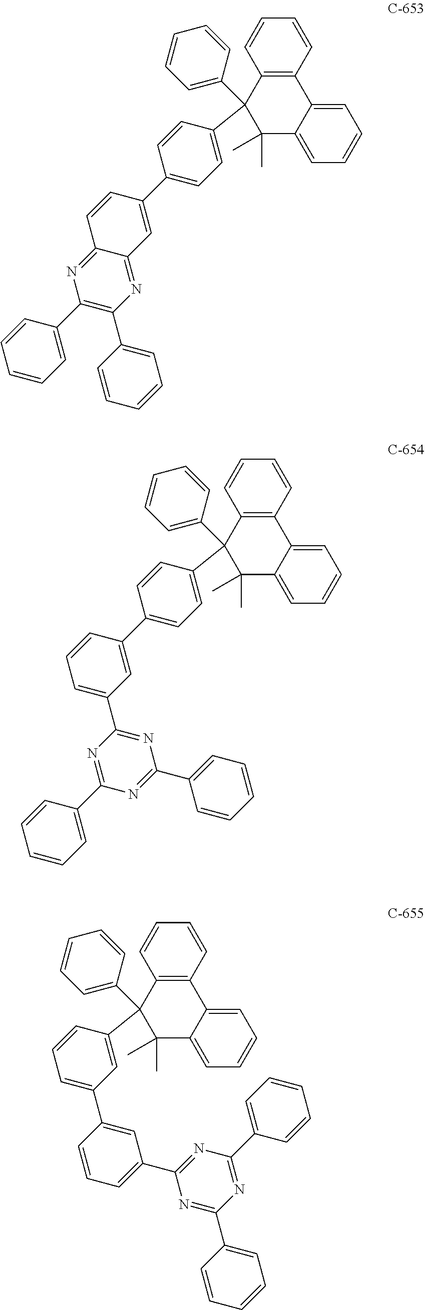 [SMILES: CC1(C)c2ccccc2-c2ccccc2C1(c1ccccc1)c1ccc(-c2ccc3nc(-c4ccccc4)c(-c4ccccc4)nc3c2)cc1 ; CC1(C)c2ccccc2-c2ccccc2C1(c1ccccc1)c1ccc(-c2cccc(-c3nc(-c4ccccc4)nc(-c4ccccc4)n3)c2)cc1 ; CC1(C)c2ccccc2-c2ccccc2C1(c1ccccc1)c1cccc(-c2cccc(-c3nc(-c4ccccc4)nc(-c4ccccc4)n3)c2)c1]

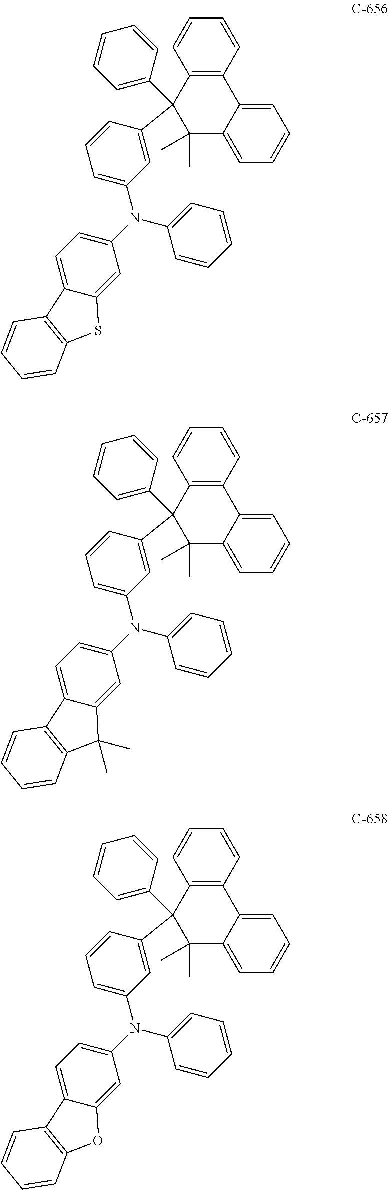 [SMILES: CC1(C)c2ccccc2-c2ccccc2C1(c1ccccc1)c1cccc(N(c2ccccc2)c2ccc3c(c2)sc2ccccc23)c1 ; CC1(C)c2ccccc2-c2ccc(N(c3ccccc3)c3cccc(C4(c5ccccc5)c5ccccc5-c5ccccc5C4(C)C)c3)cc21 ; CC1(C)c2ccccc2-c2ccccc2C1(c1ccccc1)c1cccc(N(c2ccccc2)c2ccc3c(c2)oc2ccccc23)c1]

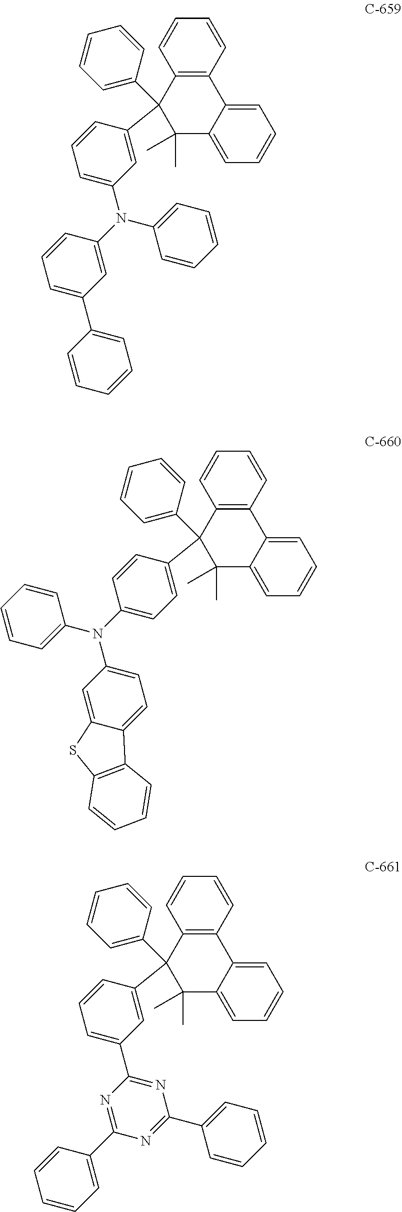 [SMILES: CC1(C)c2ccccc2-c2ccccc2C1(c1ccccc1)c1cccc(N(c2ccccc2)c2cccc(-c3ccccc3)c2)c1 ; CC1(C)c2ccccc2-c2ccccc2C1(c1ccccc1)c1ccc(N(c2ccccc2)c2ccc3c(c2)sc2ccccc23)cc1 ; CC1(C)c2ccccc2-c2ccccc2C1(c1ccccc1)c1cccc(-c2nc(-c3ccccc3)nc(-c3ccccc3)n2)c1]

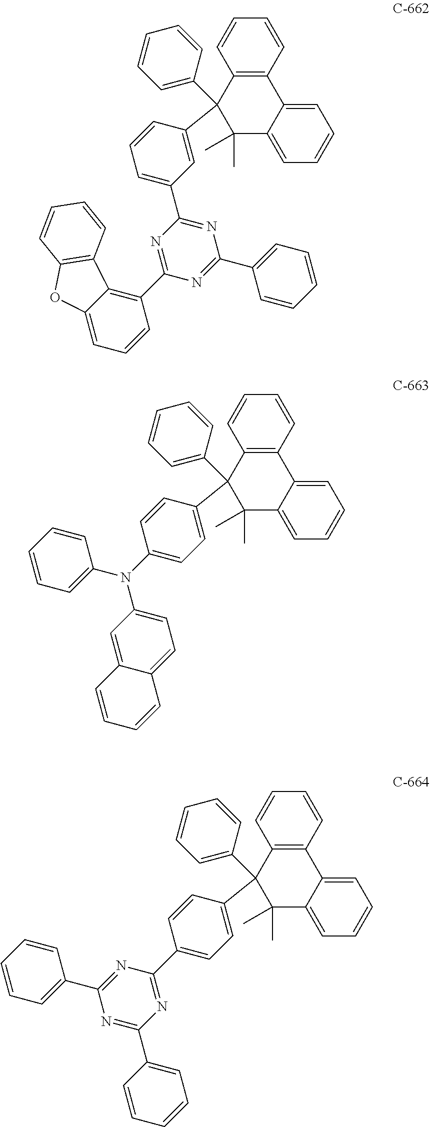 [SMILES: CC1(C)c2ccccc2-c2ccccc2C1(c1ccccc1)c1cccc(-c2nc(-c3ccccc3)nc(-c3cccc4oc5ccccc5c34)n2)c1 ; CC1(C)c2ccccc2-c2ccccc2C1(c1ccccc1)c1ccc(N(c2ccccc2)c2ccc3ccccc3c2)cc1 ; CC1(C)c2ccccc2-c2ccccc2C1(c1ccccc1)c1ccc(-c2nc(-c3ccccc3)nc(-c3ccccc3)n2)cc1]

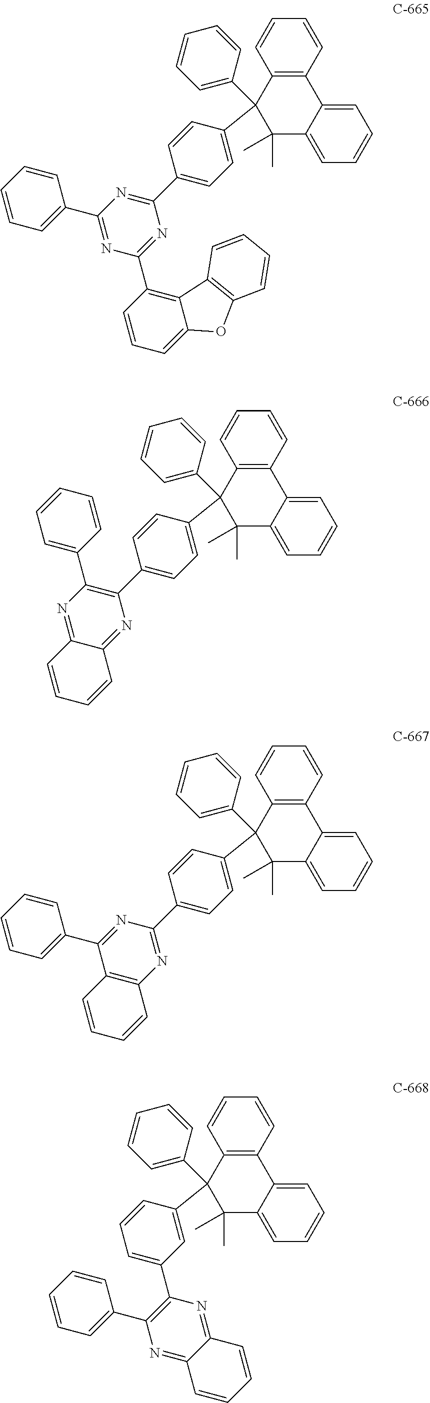 [SMILES: CC1(C)c2ccccc2-c2ccccc2C1(c1ccccc1)c1ccc(-c2nc(-c3ccccc3)nc(-c3cccc4oc5ccccc5c34)n2)cc1 ; CC1(C)c2ccccc2-c2ccccc2C1(c1ccccc1)c1ccc(-c2nc3ccccc3nc2-c2ccccc2)cc1 ; CC1(C)c2ccccc2-c2ccccc2C1(c1ccccc1)c1ccc(-c2nc(-c3ccccc3)c3ccccc3n2)cc1 ; CC1(C)c2ccccc2-c2ccccc2C1(c1ccccc1)c1cccc(-c2nc3ccccc3nc2-c2ccccc2)c1]

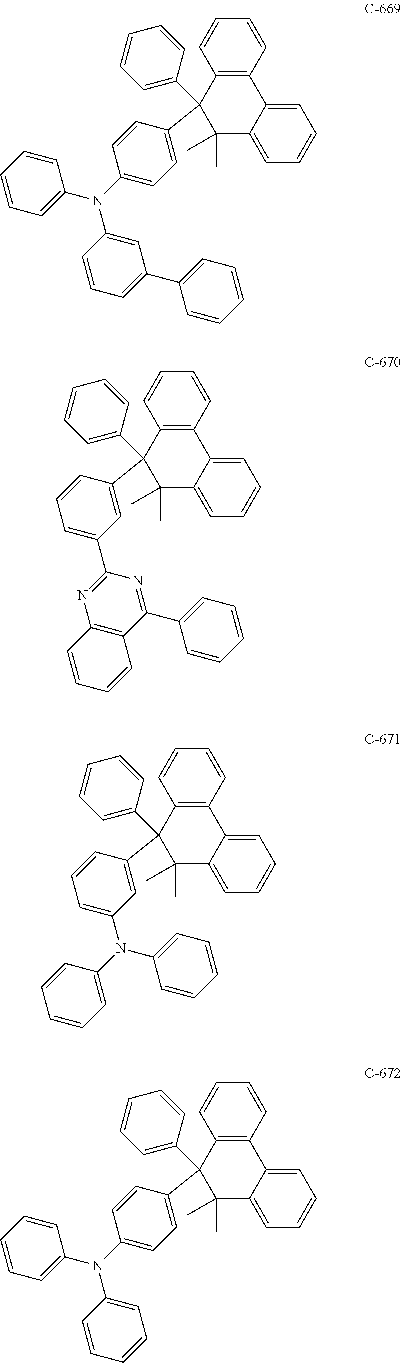 [SMILES: CC1(C)c2ccccc2-c2ccccc2C1(c1ccccc1)c1ccc(N(c2ccccc2)c2cccc(-c3ccccc3)c2)cc1 ; CC1(C)c2ccccc2-c2ccccc2C1(c1ccccc1)c1cccc(-c2nc(-c3ccccc3)c3ccccc3n2)c1 ; CC1(C)c2ccccc2-c2ccccc2C1(c1ccccc1)c1cccc(N(c2ccccc2)c2ccccc2)c1 ; CC1(C)c2ccccc2-c2ccccc2C1(c1ccccc1)c1ccc(N(c2ccccc2)c2ccccc2)cc1]

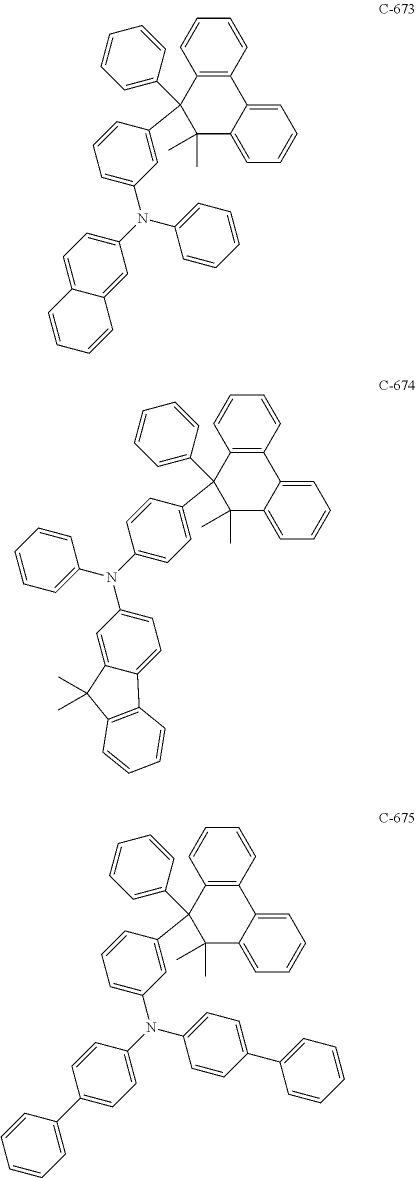 [SMILES: CC1(C)c2ccccc2-c2ccccc2C1(c1ccccc1)c1cccc(N(c2ccccc2)c2ccc3ccccc3c2)c1 ; CC1(C)c2ccccc2-c2ccc(N(c3ccccc3)c3ccc(C4(c5ccccc5)c5ccccc5-c5ccccc5C4(C)C)cc3)cc21 ; CC1(C)c2ccccc2-c2ccccc2C1(c1ccccc1)c1cccc(N(c2ccc(-c3ccccc3)cc2)c2ccc(-c3ccccc3)cc2)c1]

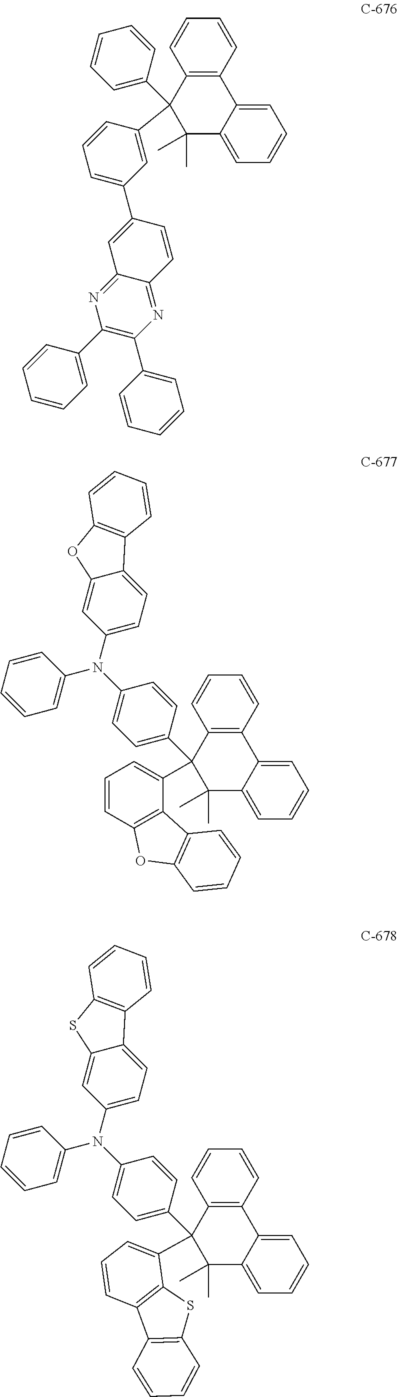 [SMILES: CC1(C)c2ccccc2-c2ccccc2C1(c1ccccc1)c1cccc(-c2ccc3nc(-c4ccccc4)c(-c4ccccc4)nc3c2)c1 ; CC1(C)c2ccccc2-c2ccccc2C1(c1ccc(N(c2ccccc2)c2ccc3c(c2)oc2ccccc23)cc1)c1cccc2oc3ccccc3c12 ; CC1(C)c2ccccc2-c2ccccc2C1(c1ccc(N(c2ccccc2)c2ccc3c(c2)sc2ccccc23)cc1)c1cccc2c1sc1ccccc12]

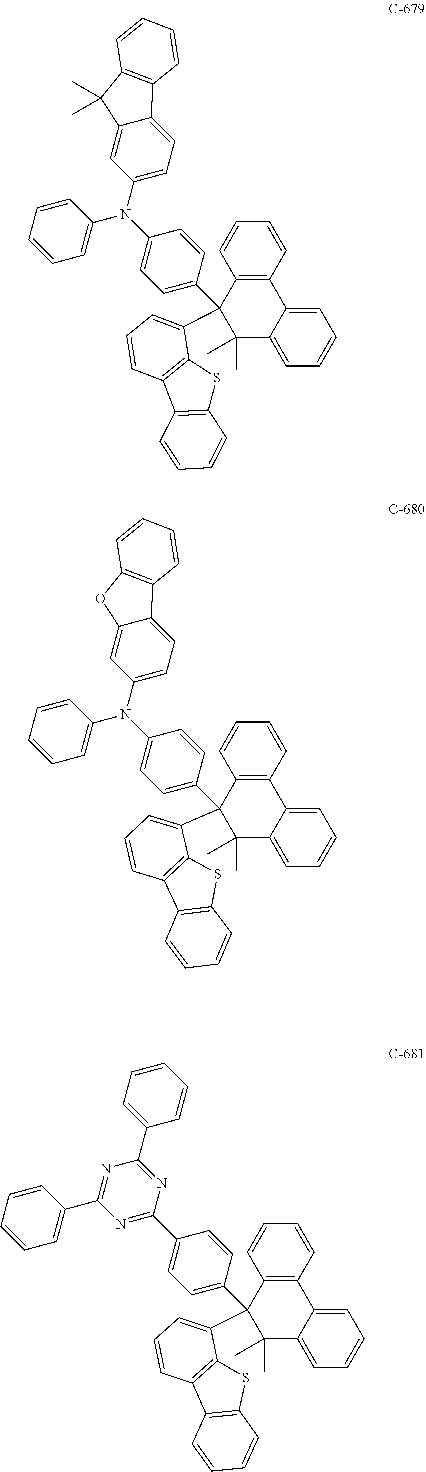 [SMILES: CC1(C)c2ccccc2-c2ccc(N(c3ccccc3)c3ccc(C4(c5cccc6c5sc5ccccc56)c5ccccc5-c5ccccc5C4(C)C)cc3)cc21 ; CC1(C)c2ccccc2-c2ccccc2C1(c1ccc(N(c2ccccc2)c2ccc3c(c2)oc2ccccc23)cc1)c1cccc2c1sc1ccccc12 ; CC1(C)c2ccccc2-c2ccccc2C1(c1ccc(-c2nc(-c3ccccc3)nc(-c3ccccc3)n2)cc1)c1cccc2c1sc1ccccc12]

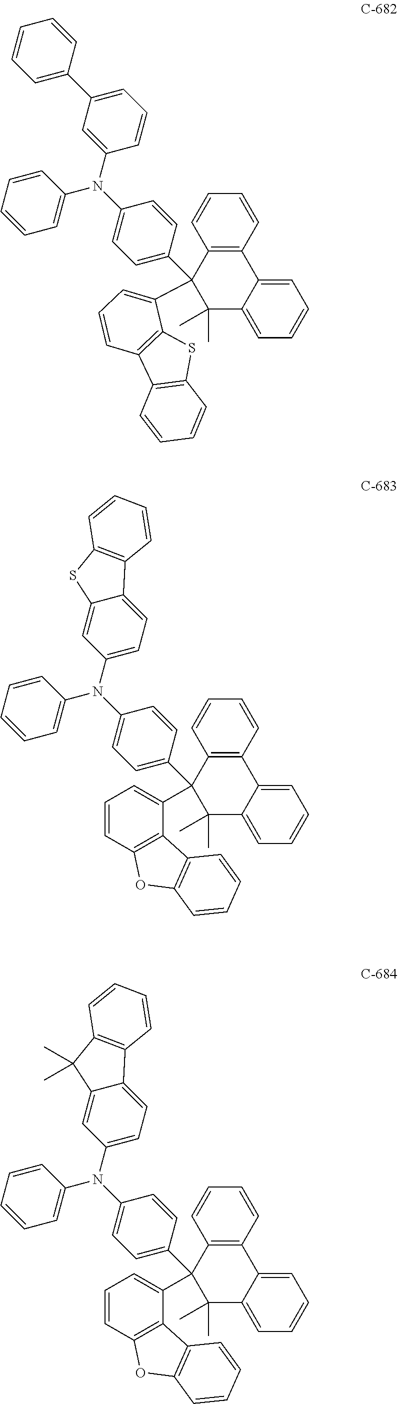 [SMILES: CC1(C)c2ccccc2-c2ccccc2C1(c1ccc(N(c2ccccc2)c2cccc(-c3ccccc3)c2)cc1)c1cccc2c1sc1ccccc12 ; CC1(C)c2ccccc2-c2ccccc2C1(c1ccc(N(c2ccccc2)c2ccc3c(c2)sc2ccccc23)cc1)c1cccc2oc3ccccc3c12 ; CC1(C)c2ccccc2-c2ccc(N(c3ccccc3)c3ccc(C4(c5cccc6oc7ccccc7c56)c5ccccc5-c5ccccc5C4(C)C)cc3)cc21]

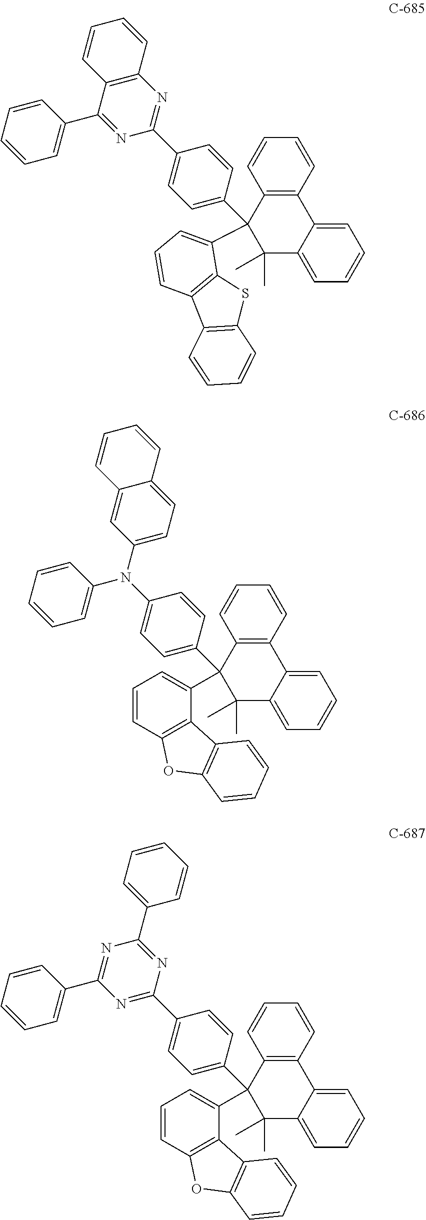 [SMILES: CC1(C)c2ccccc2-c2ccccc2C1(c1ccc(-c2nc(-c3ccccc3)c3ccccc3n2)cc1)c1cccc2c1sc1ccccc12 ; CC1(C)c2ccccc2-c2ccccc2C1(c1ccc(N(c2ccccc2)c2ccc3ccccc3c2)cc1)c1cccc2oc3ccccc3c12 ; CC1(C)c2ccccc2-c2ccccc2C1(c1ccc(-c2nc(-c3ccccc3)nc(-c3ccccc3)n2)cc1)c1cccc2oc3ccccc3c12]

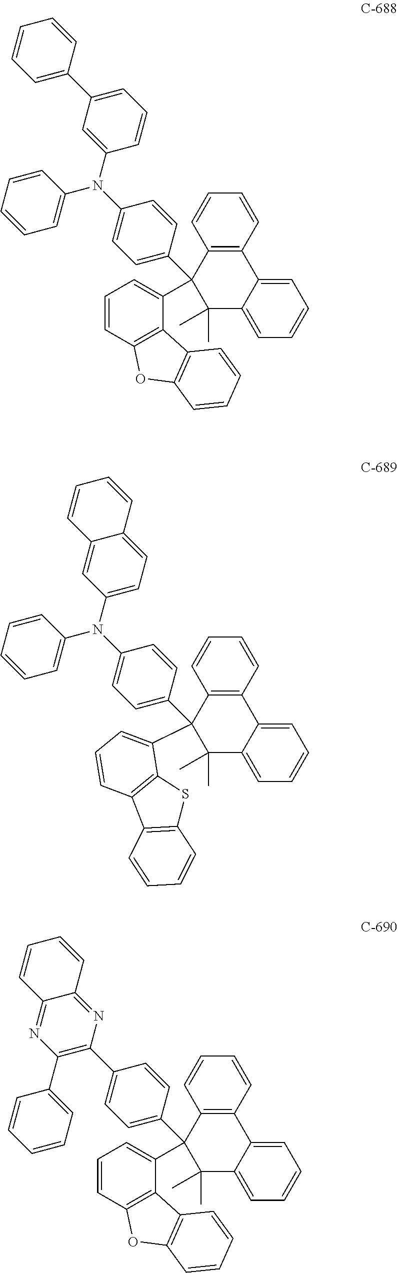 [SMILES: CC1(C)c2ccccc2-c2ccccc2C1(c1ccc(N(c2ccccc2)c2cccc(-c3ccccc3)c2)cc1)c1cccc2oc3ccccc3c12 ; CC1(C)c2ccccc2-c2ccccc2C1(c1ccc(N(c2ccccc2)c2ccc3ccccc3c2)cc1)c1cccc2c1sc1ccccc12 ; CC1(C)c2ccccc2-c2ccccc2C1(c1ccc(-c2nc3ccccc3nc2-c2ccccc2)cc1)c1cccc2oc3ccccc3c12]

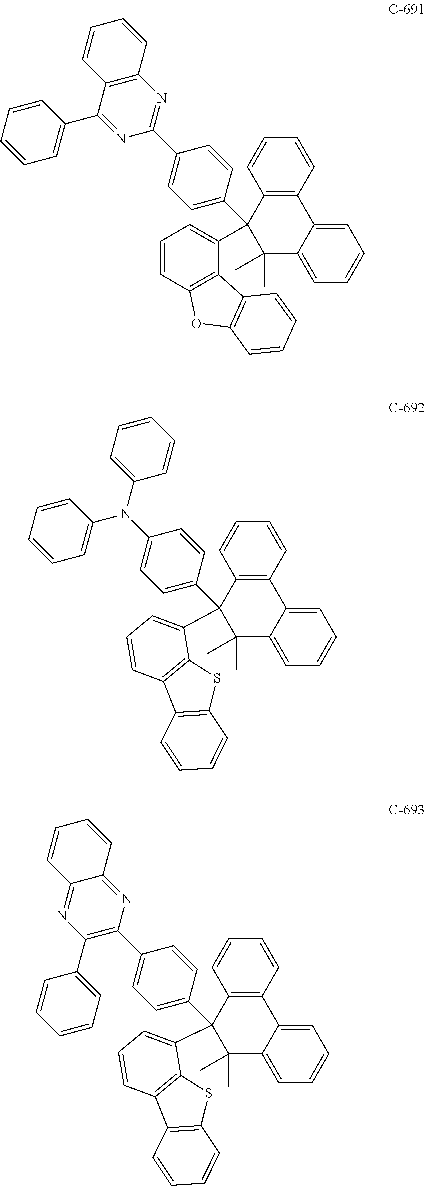 [SMILES: CC1(C)c2ccccc2-c2ccccc2C1(c1ccc(-c2nc(-c3ccccc3)c3ccccc3n2)cc1)c1cccc2oc3ccccc3c12 ; CC1(C)c2ccccc2-c2ccccc2C1(c1ccc(N(c2ccccc2)c2ccccc2)cc1)c1cccc2c1sc1ccccc12 ; CC1(C)c2ccccc2-c2ccccc2C1(c1ccc(-c2nc3ccccc3nc2-c2ccccc2)cc1)c1cccc2c1sc1ccccc12]

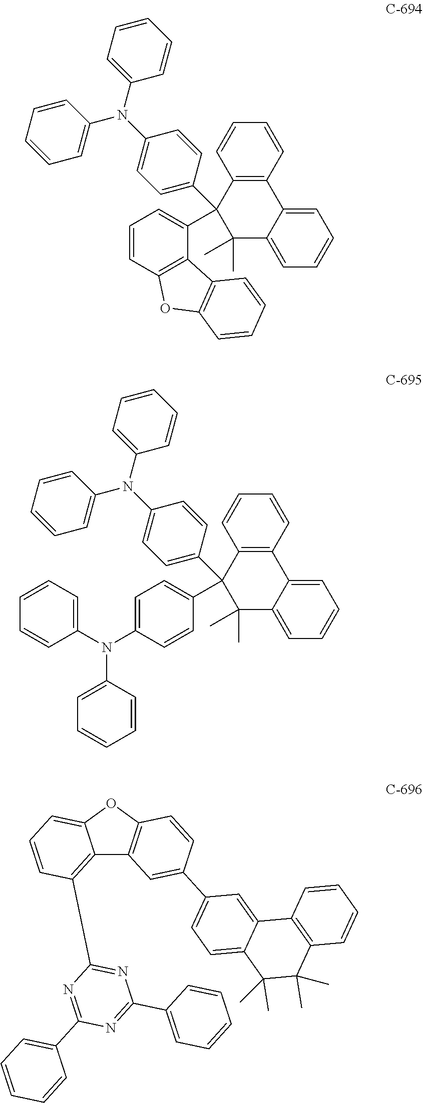 [SMILES: CC1(C)c2ccccc2-c2ccccc2C1(c1ccc(N(c2ccccc2)c2ccccc2)cc1)c1cccc2oc3ccccc3c12 ; CC1(C)c2ccccc2-c2ccccc2C1(c1ccc(N(c2ccccc2)c2ccccc2)cc1)c1ccc(N(c2ccccc2)c2ccccc2)cc1 ; CC1(C)c2ccccc2-c2cc(-c3ccc4oc5cccc(-c6nc(-c7ccccc7)nc(-c7ccccc7)n6)c5c4c3)ccc2C1(C)C]

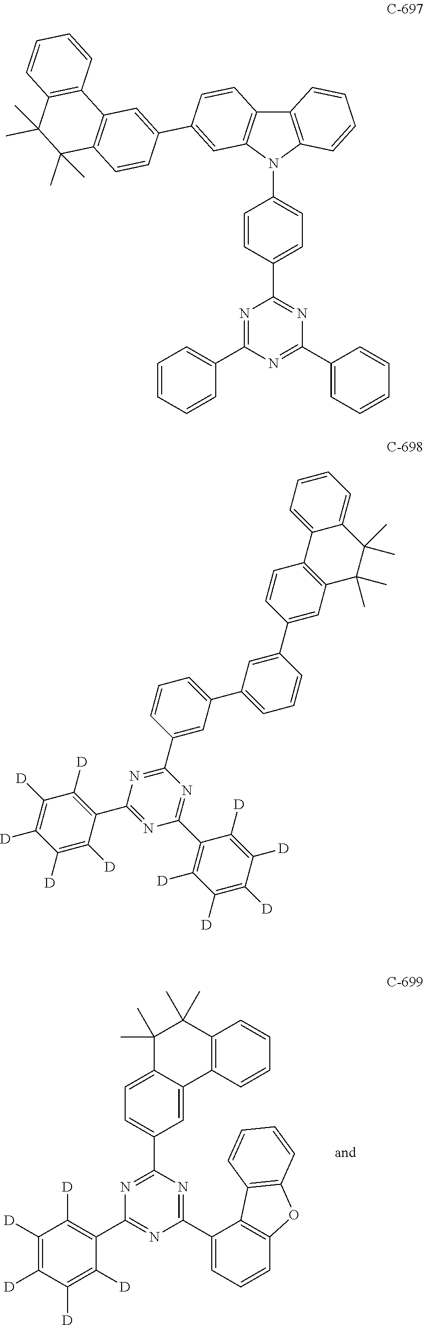 [SMILES: CC1(C)c2ccccc2-c2cc(-c3ccc4c5ccccc5n(-c5ccc(-c6nc(-c7ccccc7)nc(-c7ccccc7)n6)cc5)c4c3)ccc2C1(C)C ; [2H]c1c([2H])c([2H])c(-c2nc(-c3cccc(-c4cccc(-c5ccc6c(c5)C(C)(C)C(C)(C)c5ccccc5-6)c4)c3)nc(-c3c([2H])c([2H])c([2H])c([2H])c3[2H])n2)c([2H])c1[2H] ; [2H]c1c([2H])c([2H])c(-c2nc(-c3ccc4c(c3)-c3ccccc3C(C)(C)C4(C)C)nc(-c3cccc4oc5ccccc5c34)n2)c([2H])c1[2H]]

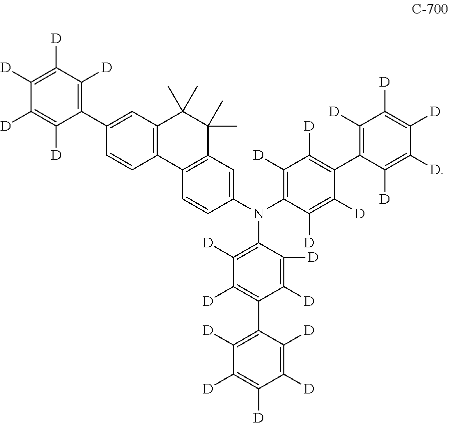

10. An organic electroluminescent material comprising the organic electroluminescent compound according to claim 1.

11. A plurality of host materials comprising at least one organic electroluminescent material according to claim 10 as a first host material and at least one second host material which is different from the first host material.

12. The plurality of host materials according to claim 11, wherein the second host material comprises a compound represented by the following formula 11: 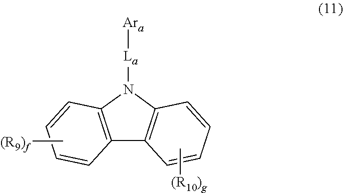
wherein
Lₐ represents a single bond, a substituted or unsubstituted (C6-C30)arylene, or a substituted or unsubstituted (3- to 30-membered)heteroarylene;
Arₐ represents a substituted or unsubstituted (C6-C30)aryl, or a substituted or unsubstituted (3- to 30-membered)heteroaryl;
R₉ and R₁₀ each independently represent hydrogen, deuterium, halogen, cyano, a substituted or unsubstituted (C1-C30)alkyl, a substituted or unsubstituted (C6-C30)aryl, a substituted or unsubstituted (3- to 50-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of an (C3-C30) aliphatic ring and a (C6-C30) aromatic ring, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to the adjacent substituent(s) to form a ring(s); and
f and g each independently represent an integer of 1 to 4; and when f and g are 2 or more, each of R₉ and each of R₁₀ may be the same or different.

13. The plurality of host materials according to claim 12, wherein the compound represented by formula 11 is represented by the following formula 12 or 13: 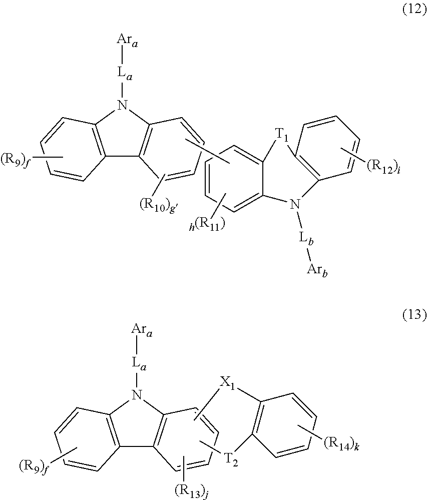
wherein
Lₐ, Arₐ, R₉, R₁₀, and f are as defined in claim 12;
T₁ and T₂ each independently represent a single bond, O, or S;
L_{b} is as defined as Lₐ in claim 12;
Ar_{b} is as defined as Arₐ in claim 12;
R₁₁ to R₁₄ each independently are as defined as R₉ in claim 12;
X₁ represents O, S, or NRₐ;
Rₐ represents a substituted or unsubstituted (C6-C30)aryl; and
g′ and h each independently represent an integer of 1 to 3, i and k each independently represent an integer of 1 to 4, and j represents an integer of 1 or 2; and when g′, h, i, j, and k are 2 or more, each of R₁₀, each of R₁₁, each of R₁₂, each of R₁₃, and each of R₁₄ may be the same or different.

14. The plurality of host materials according to claim 12, wherein the compound represented by formula 11 is selected from the following compounds:

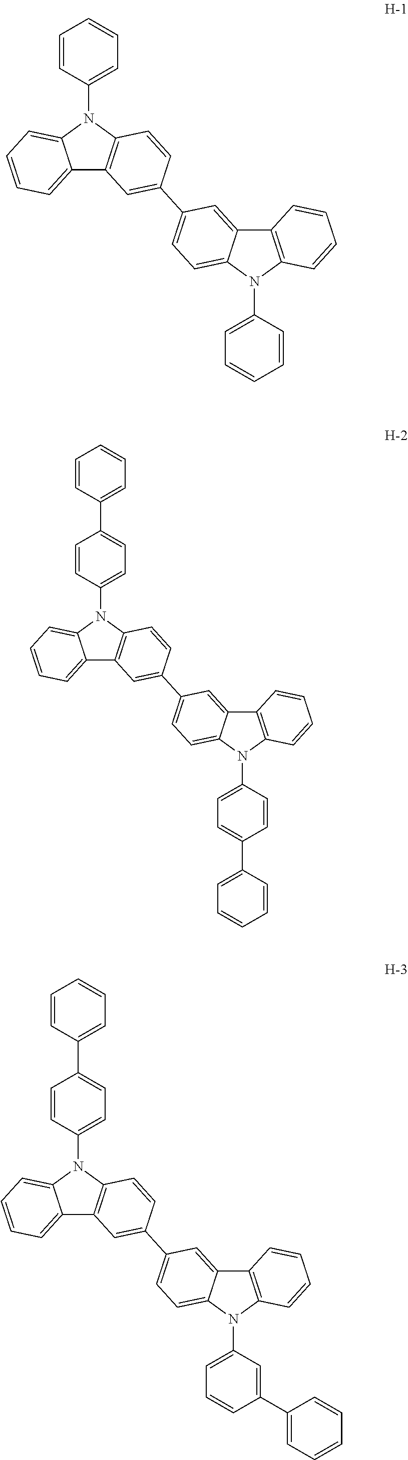 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

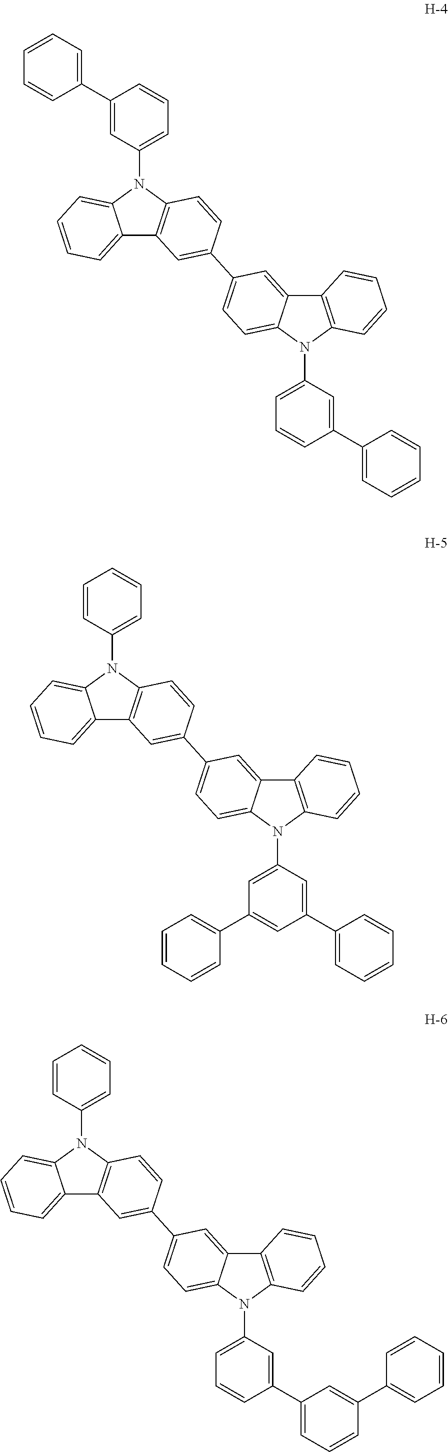 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1]

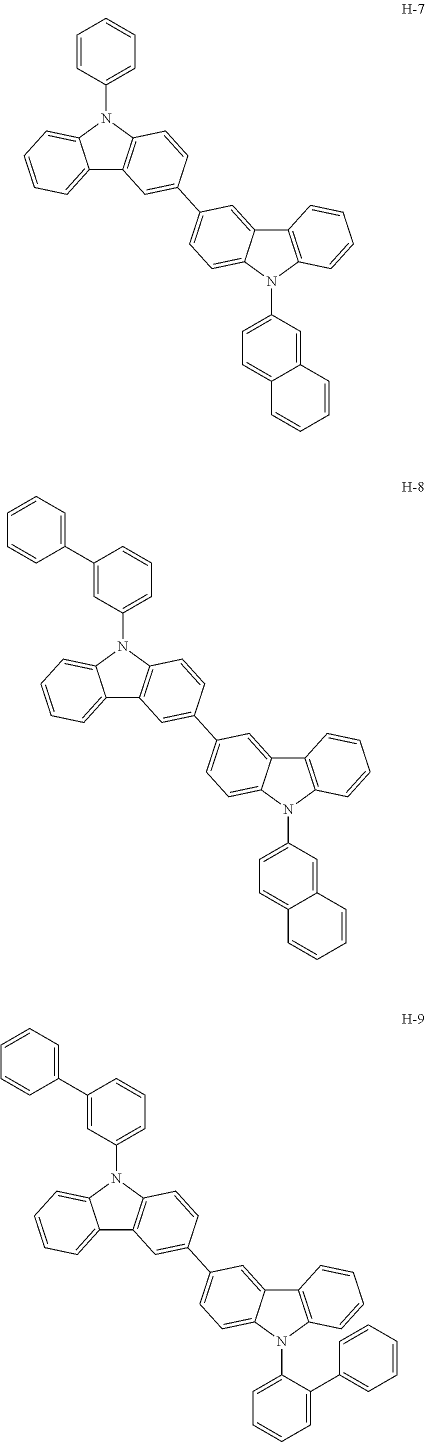 [SMILES: c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1]

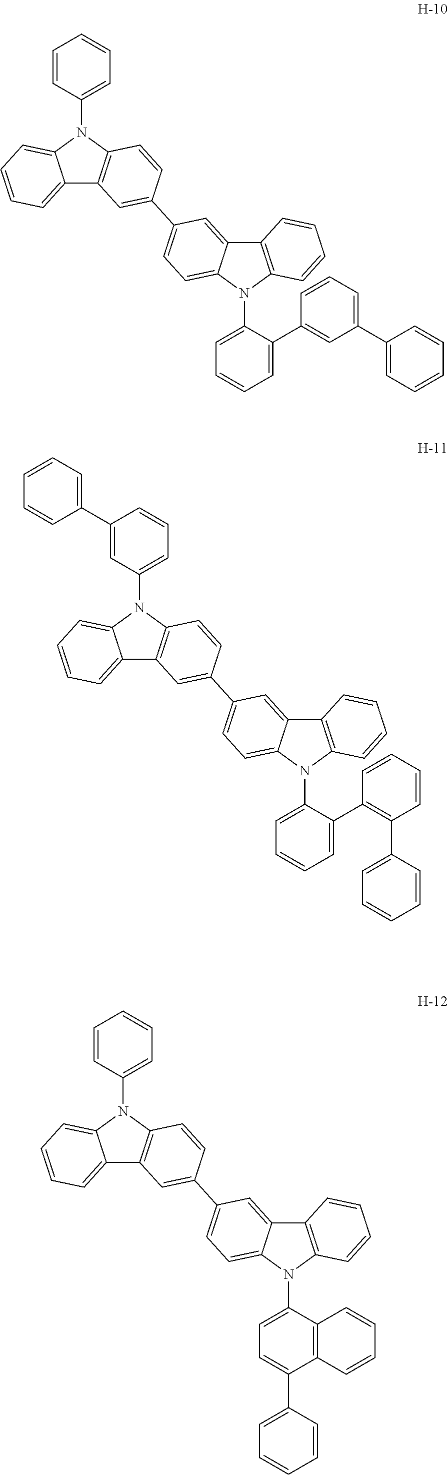 [SMILES: c1ccc(-c2cccc(-c3ccccc3-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccccc5-c5ccccc5-c5ccccc5)ccc43)c2)cc1 ; c1ccc(-c2ccc(-n3c4ccccc4c4cc(-c5ccc6c(c5)c5ccccc5n6-c5ccccc5)ccc43)c3ccccc23)cc1]

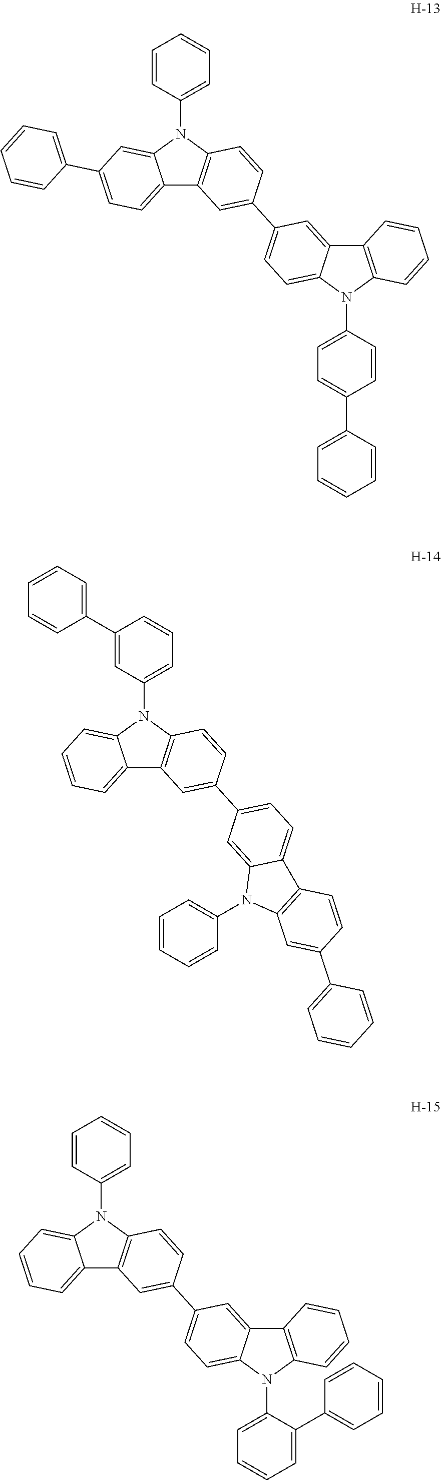 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2cccc(-n3c4ccccc4c4cc(-c5ccc6c7ccc(-c8ccccc8)cc7n(-c7ccccc7)c6c5)ccc43)c2)cc1 ; c1ccc(-c2ccccc2-n2c3ccccc3c3cc(-c4ccc5c(c4)c4ccccc4n5-c4ccccc4)ccc32)cc1]

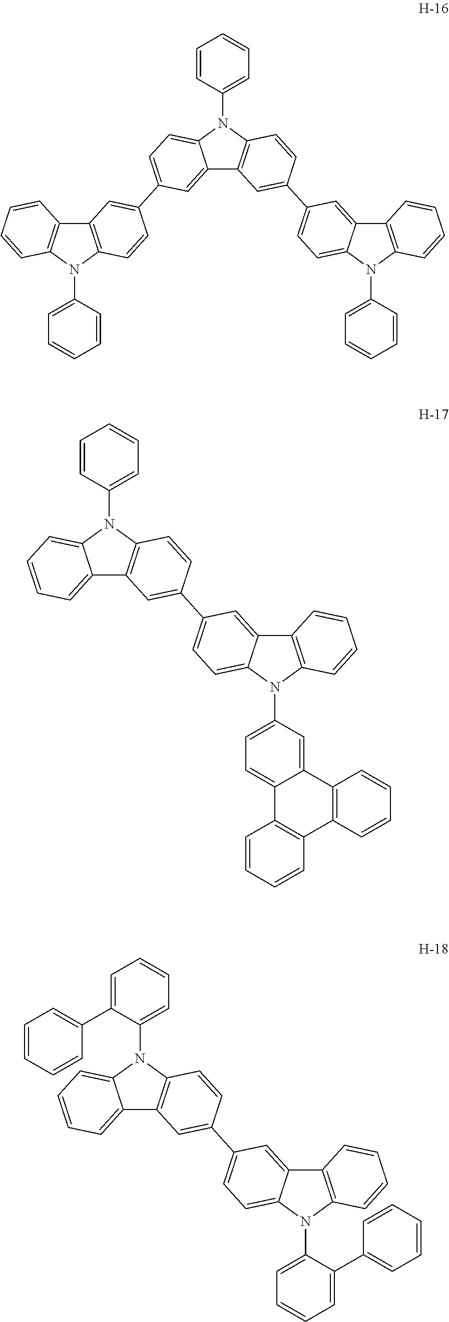 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; c1ccc(-c2ccccc2-n2c3ccccc3c3cc(-c4ccc5c(c4)c4ccccc4n5-c4ccccc4-c4ccccc4)ccc32)cc1]

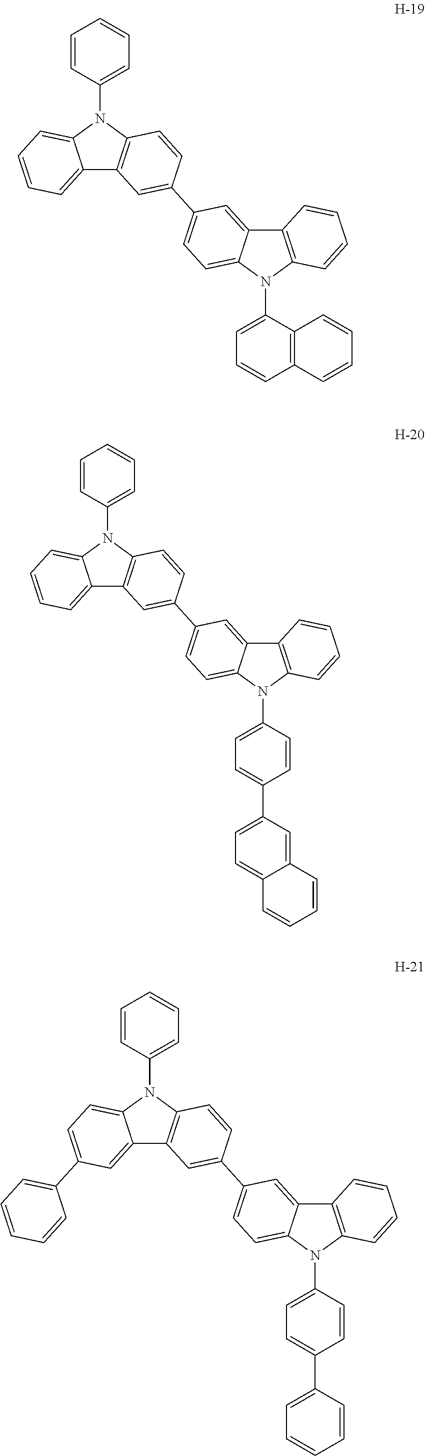 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

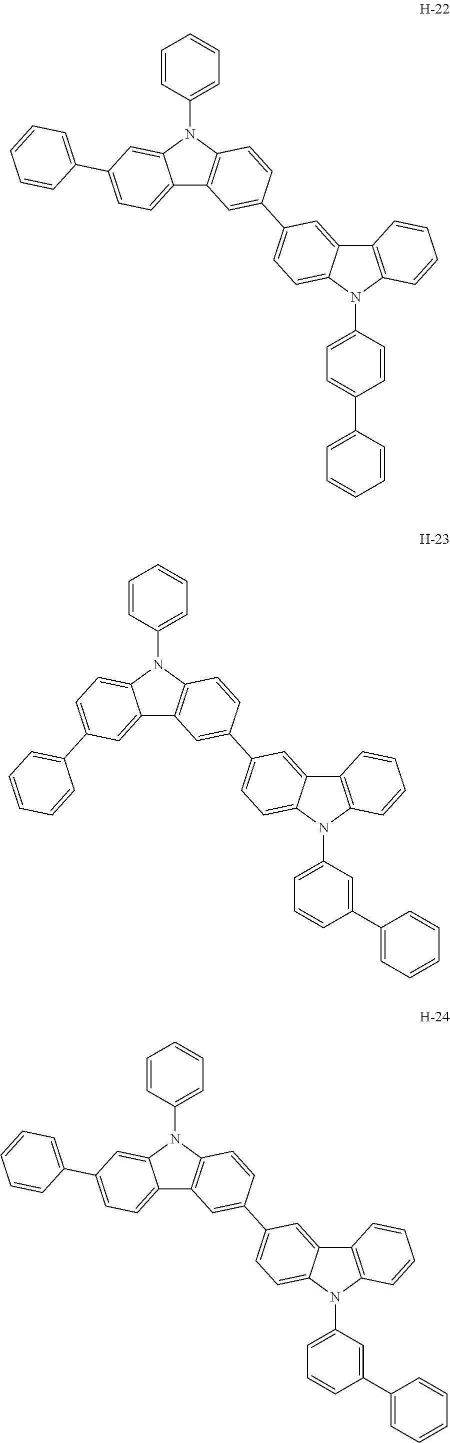 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-c2cccc(-n3c4ccccc4c4cc(-c5ccc6c(c5)c5ccc(-c7ccccc7)cc5n6-c5ccccc5)ccc43)c2)cc1]

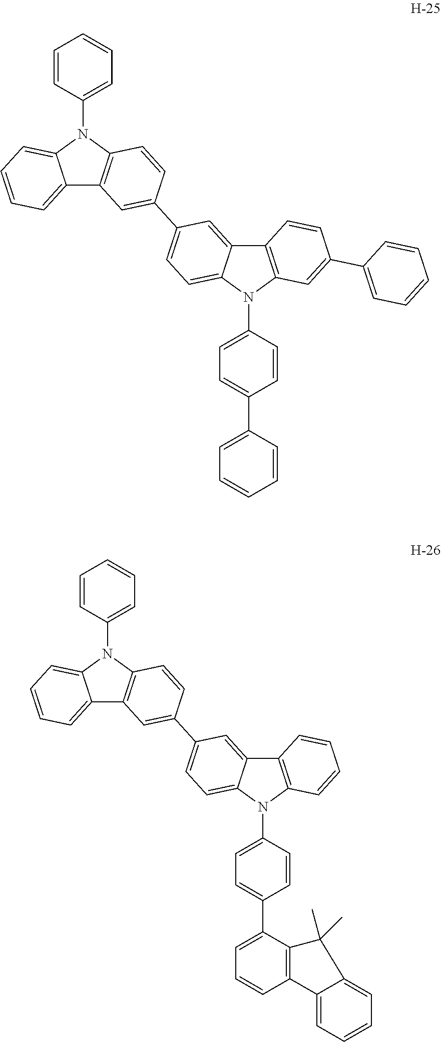 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; CC1(C)c2ccccc2-c2cccc(-c3ccc(-n4c5ccccc5c5cc(-c6ccc7c(c6)c6ccccc6n7-c6ccccc6)ccc54)cc3)c21]

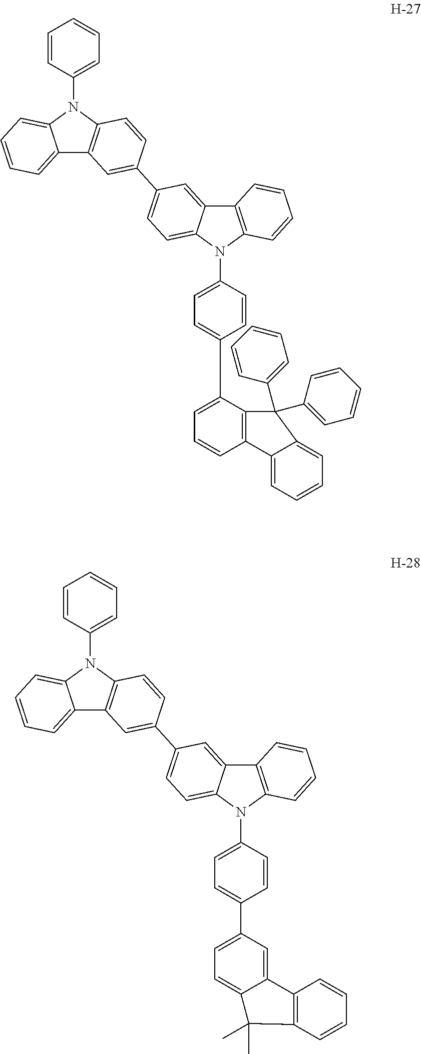 [SMILES: c1ccc(-n2c3ccccc3c3cc(-c4ccc5c(c4)c4ccccc4n5-c4ccc(-c5cccc6c5C(c5ccccc5)(c5ccccc5)c5ccccc5-6)cc4)ccc32)cc1 ; CC1(C)c2ccccc2-c2cc(-c3ccc(-n4c5ccccc5c5cc(-c6ccc7c(c6)c6ccccc6n7-c6ccccc6)ccc54)cc3)ccc21]

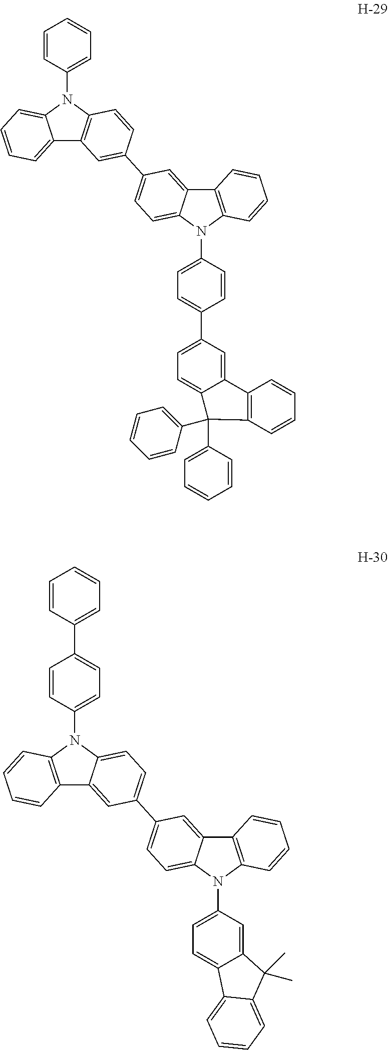 [SMILES: c1ccc(-n2c3ccccc3c3cc(-c4ccc5c(c4)c4ccccc4n5-c4ccc(-c5ccc6c(c5)-c5ccccc5C6(c5ccccc5)c5ccccc5)cc4)ccc32)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

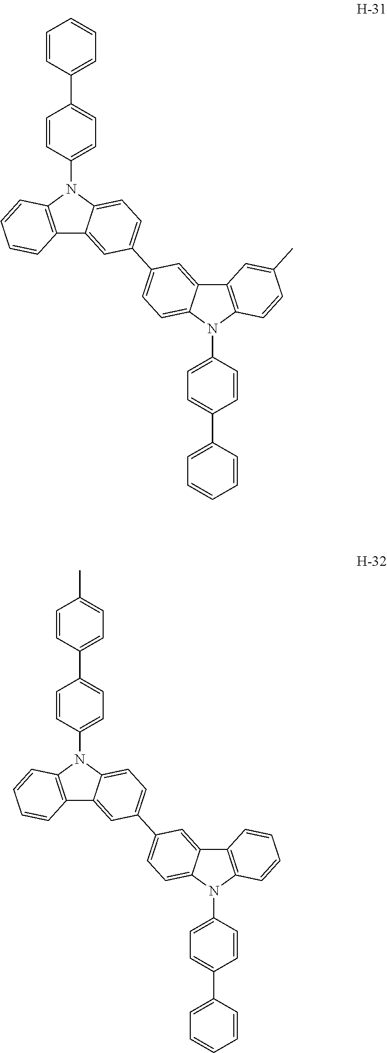 [SMILES: Cc1ccc2c(c1)c1cc(-c3ccc4c(c3)c3ccccc3n4-c3ccc(-c4ccccc4)cc3)ccc1n2-c1ccc(-c2ccccc2)cc1 ; Cc1ccc(-c2ccc(-n3c4ccccc4c4cc(-c5ccc6c(c5)c5ccccc5n6-c5ccc(-c6ccccc6)cc5)ccc43)cc2)cc1]

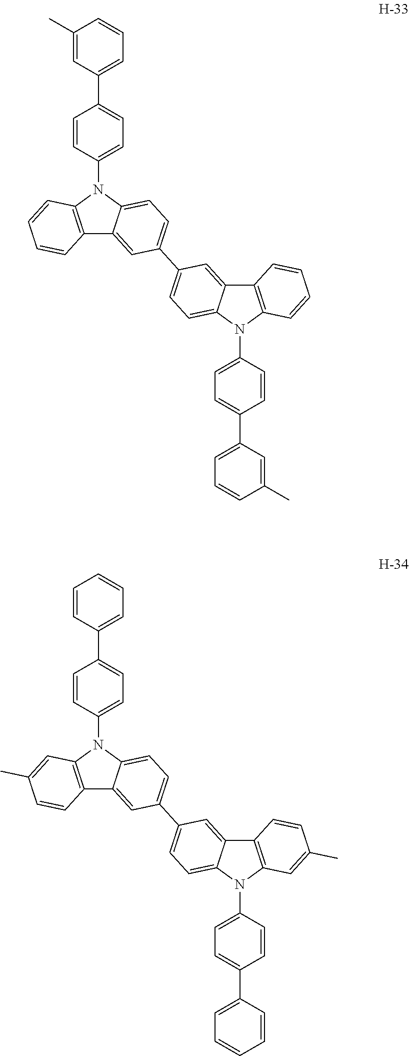

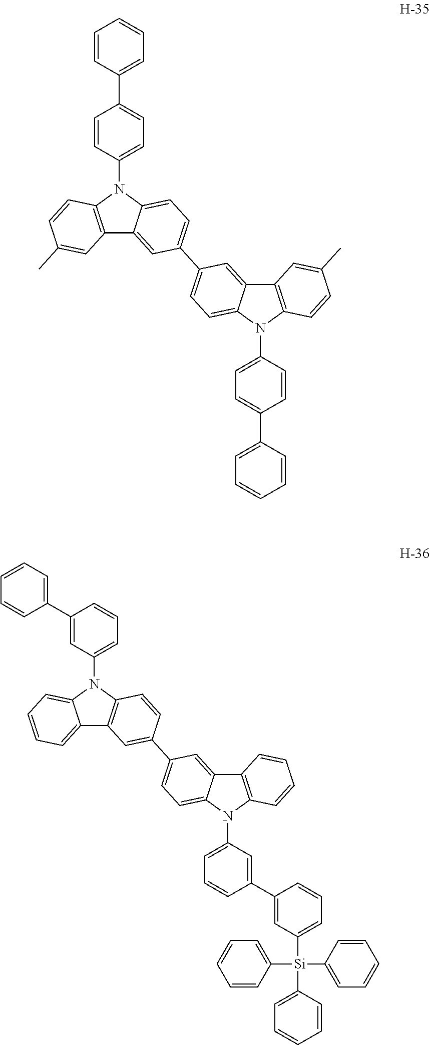

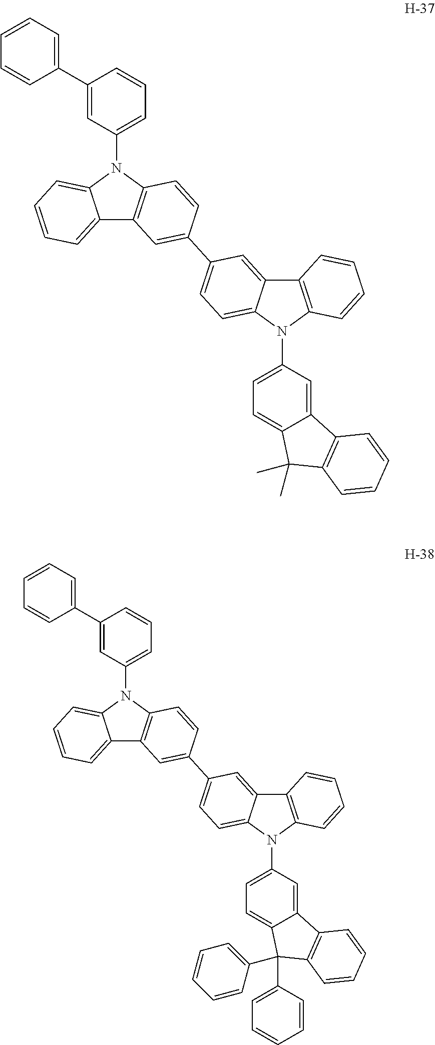 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; c1ccc(-c2cccc(-n3c4ccccc4c4cc(-c5ccc6c(c5)c5ccccc5n6-c5ccc6c(c5)-c5ccccc5C6(c5ccccc5)c5ccccc5)ccc43)c2)cc1]

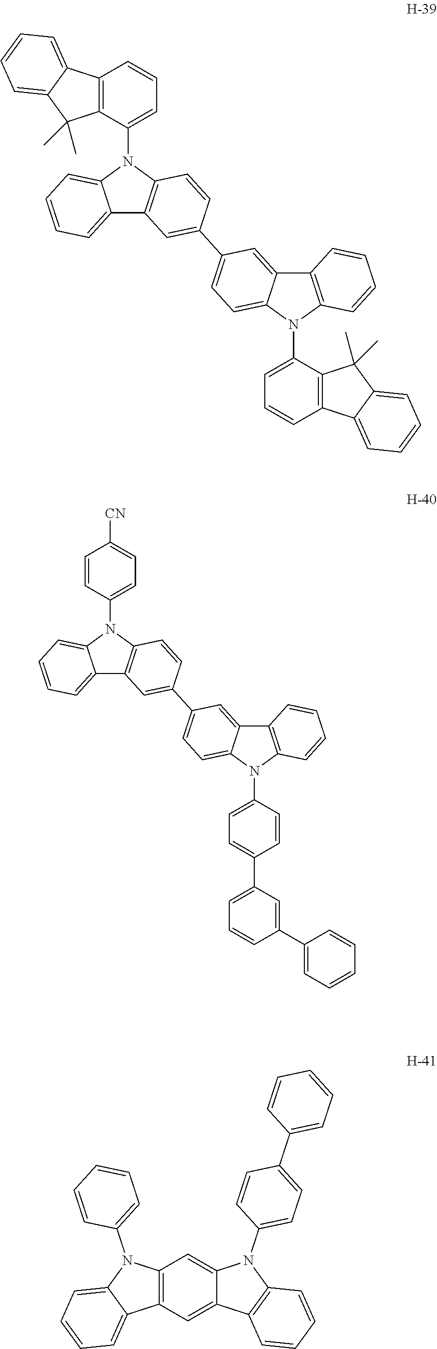 [SMILES: CC1(C)c2ccccc2-c2cccc(-n3c4ccccc4c4cc(-c5ccc6c(c5)c5ccccc5n6-c5cccc6c5C(C)(C)c5ccccc5-6)ccc43)c21 ; N#Cc1ccc(-n2c3ccccc3c3cc(-c4ccc5c(c4)c4ccccc4n5-c4ccc(-c5cccc(-c6ccccc6)c5)cc4)ccc32)cc1 ; c1ccc(-c2ccc(-n3c4ccccc4c4cc5c6ccccc6n(-c6ccccc6)c5cc43)cc2)cc1]

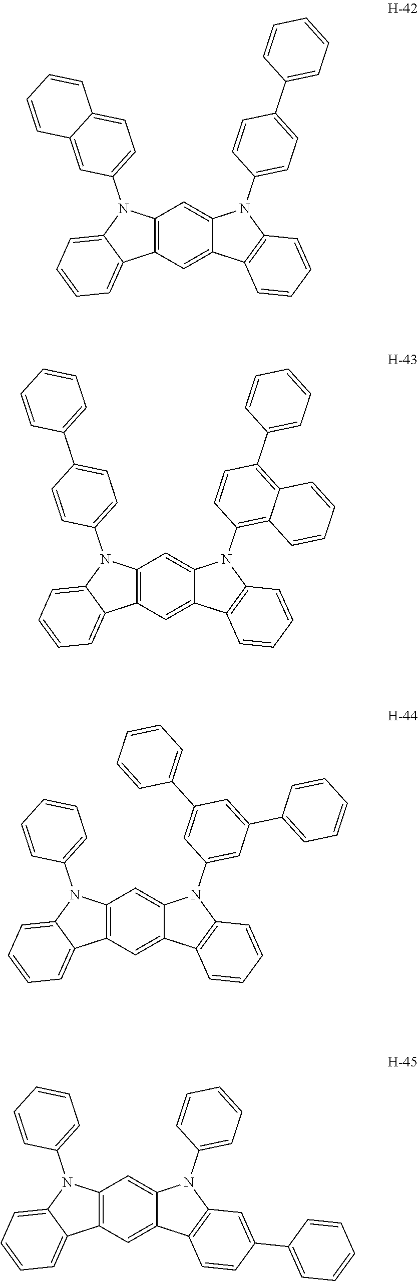 [SMILES: c1ccc(-c2ccc(-n3c4ccccc4c4cc5c6ccccc6n(-c6ccc7ccccc7c6)c5cc43)cc2)cc1 ; c1ccc(-c2ccc(-n3c4ccccc4c4cc5c6ccccc6n(-c6ccc(-c7ccccc7)c7ccccc67)c5cc43)cc2)cc1 ; c1ccc(-c2cc(-c3ccccc3)cc(-n3c4ccccc4c4cc5c6ccccc6n(-c6ccccc6)c5cc43)c2)cc1 ; c1ccc(-c2ccc3c4cc5c6ccccc6n(-c6ccccc6)c5cc4n(-c4ccccc4)c3c2)cc1]

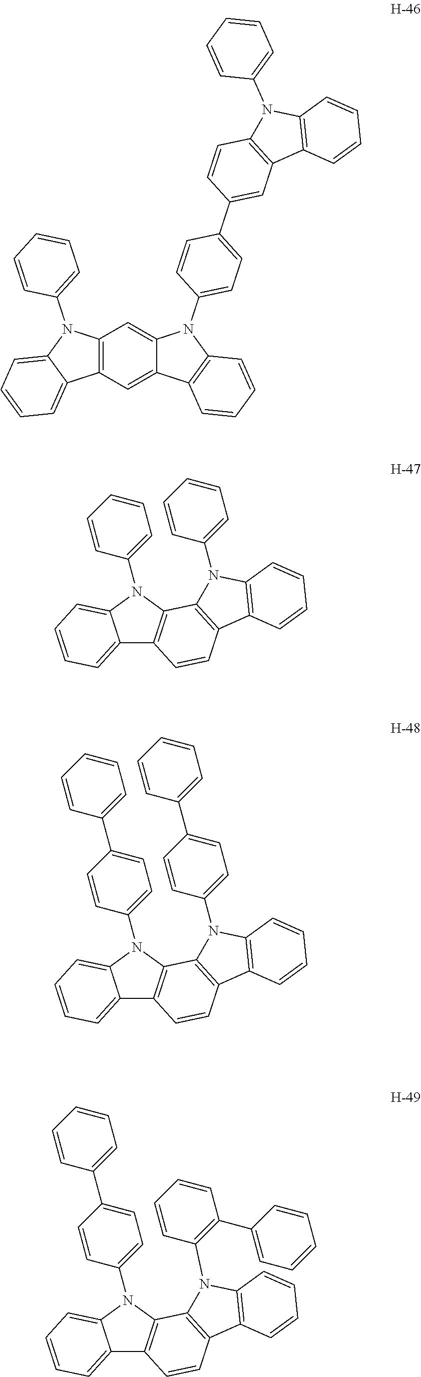 [SMILES: c1ccc(-n2c3ccccc3c3cc(-c4ccc(-n5c6ccccc6c6cc7c8ccccc8n(-c8ccccc8)c7cc65)cc4)ccc32)cc1 ; c1ccc(-n2c3ccccc3c3ccc4c5ccccc5n(-c5ccccc5)c4c32)cc1 ; c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6ccccc6-c6ccccc6)c5c43)cc2)cc1]

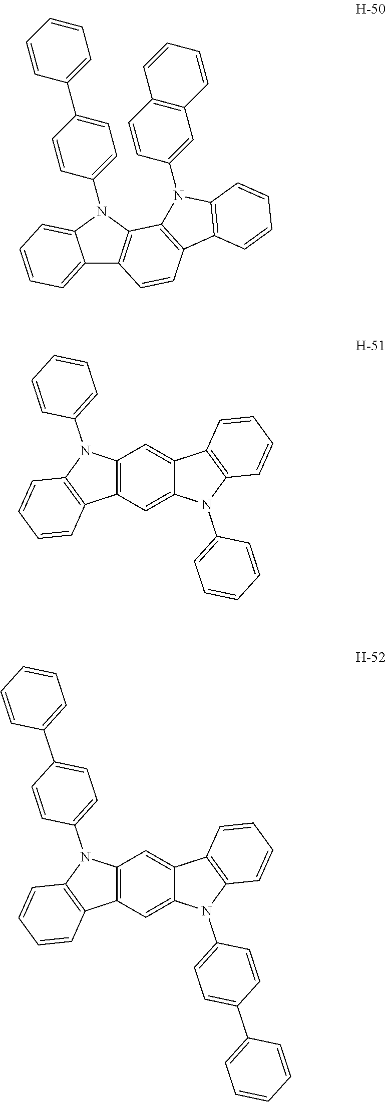 [SMILES: c1ccc(-c2ccc(-n3c4ccccc4c4ccc5c6ccccc6n(-c6ccc7ccccc7c6)c5c43)cc2)cc1 ; c1ccc(-n2c3ccccc3c3cc4c(cc32)c2ccccc2n4-c2ccccc2)cc1 ; c1ccc(-c2ccc(-n3c4ccccc4c4cc5c(cc43)c3ccccc3n5-c3ccc(-c4ccccc4)cc3)cc2)cc1]

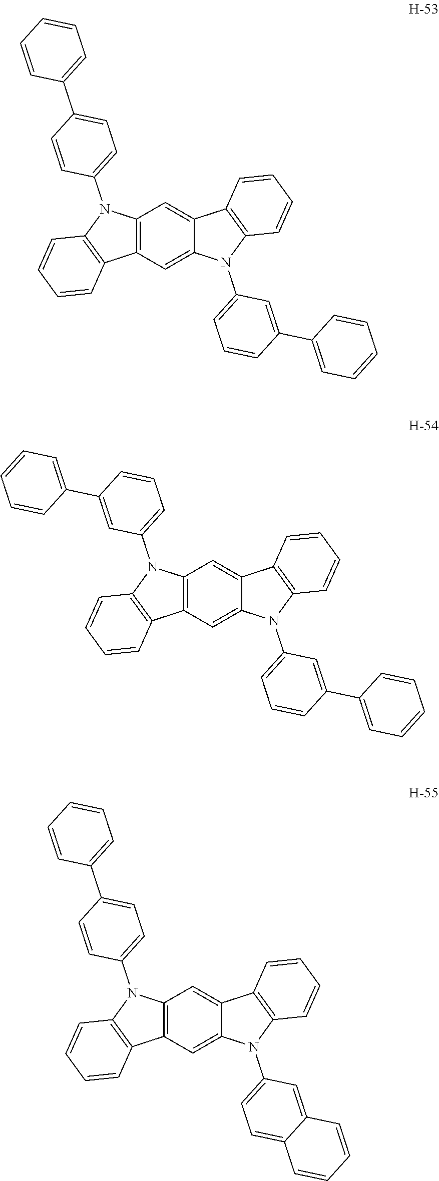 [SMILES: c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1 ; c1ccc(-c2ccc(-n3c4ccccc4c4cc5c(cc43)c3ccccc3n5-c3ccc4ccccc4c3)cc2)cc1]

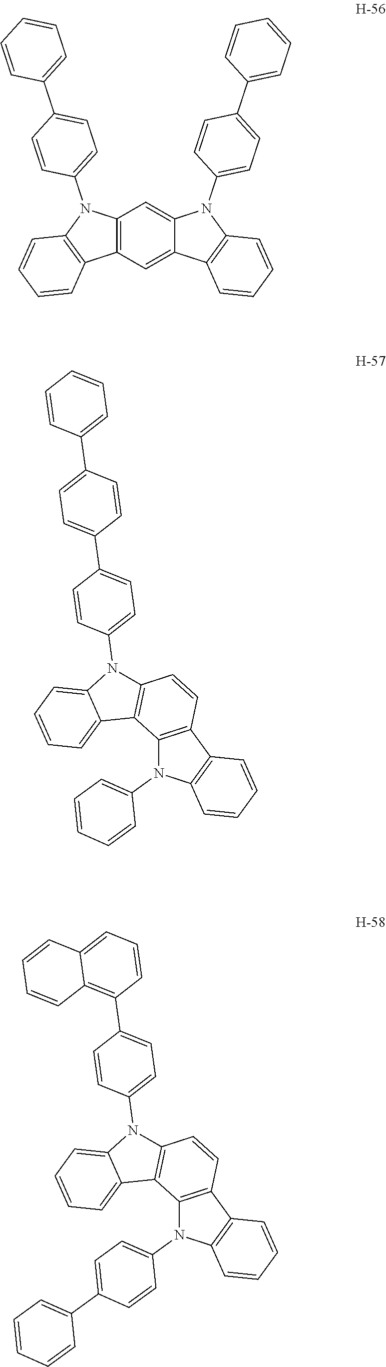 [SMILES: c1ccc(-c2ccc(-n3c4ccccc4c4cc5c6ccccc6n(-c6ccc(-c7ccccc7)cc6)c5cc43)cc2)cc1 ; c1ccc(-c2ccc(-c3ccc(-n4c5ccccc5c5c4ccc4c6ccccc6n(-c6ccccc6)c45)cc3)cc2)cc1 ; c1ccc(-c2ccc(-n3c4ccccc4c4ccc5c(c6ccccc6n5-c5ccc(-c6cccc7ccccc67)cc5)c43)cc2)cc1]

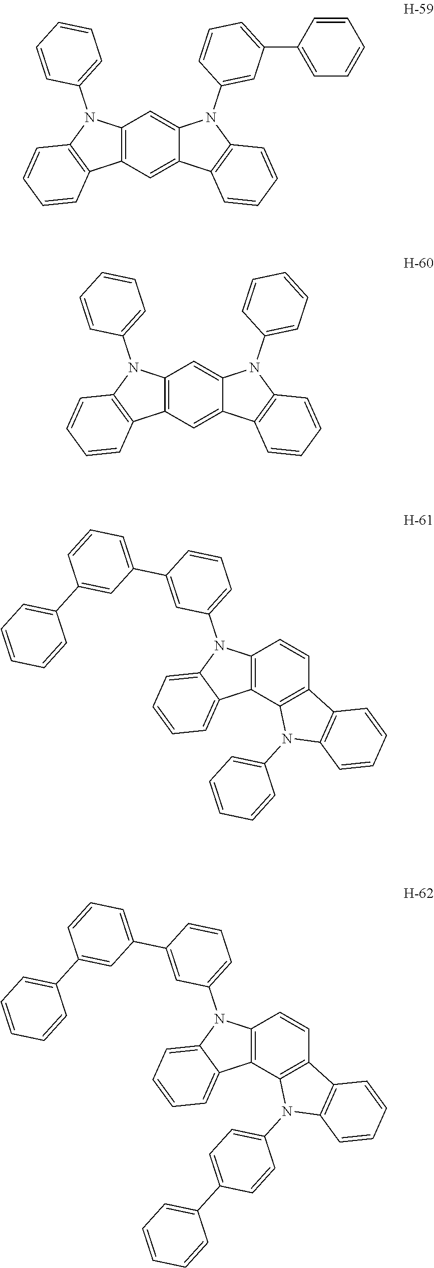 [SMILES: c1ccc(-c2cccc(-n3c4ccccc4c4cc5c6ccccc6n(-c6ccccc6)c5cc43)c2)cc1 ; c1ccc(-n2c3ccccc3c3cc4c5ccccc5n(-c5ccccc5)c4cc32)cc1 ; c1ccc(-c2cccc(-c3cccc(-n4c5ccccc5c5c4ccc4c6ccccc6n(-c6ccccc6)c45)c3)c2)cc1 ; c1ccc(-c2ccc(-n3c4ccccc4c4ccc5c(c6ccccc6n5-c5cccc(-c6cccc(-c7ccccc7)c6)c5)c43)cc2)cc1]

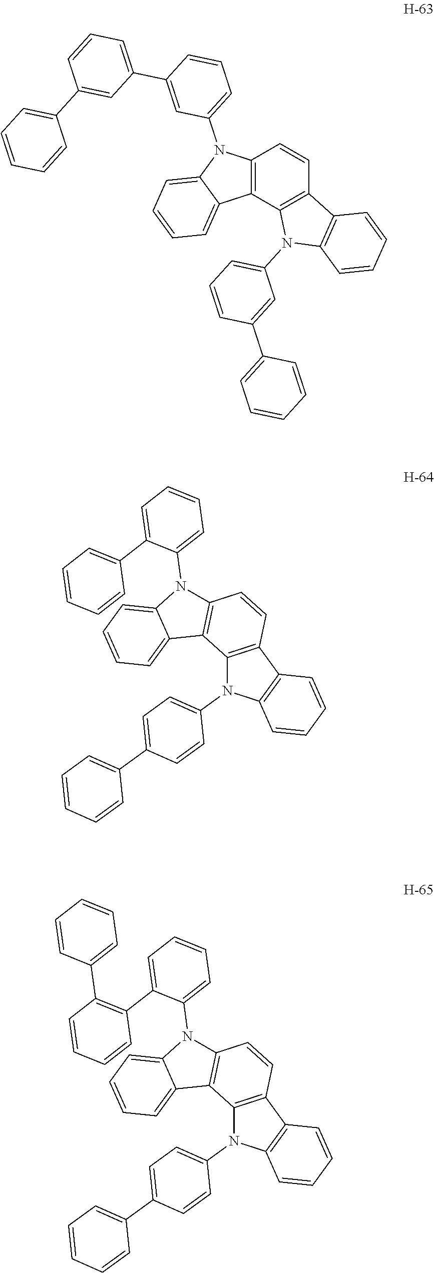 [SMILES: c1ccc(-c2cccc(-c3cccc(-n4c5ccccc5c5c4ccc4c6ccccc6n(-c6cccc(-c7ccccc7)c6)c45)c3)c2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccccc5-c5ccccc5-c5ccccc5)c43)cc2)cc1]

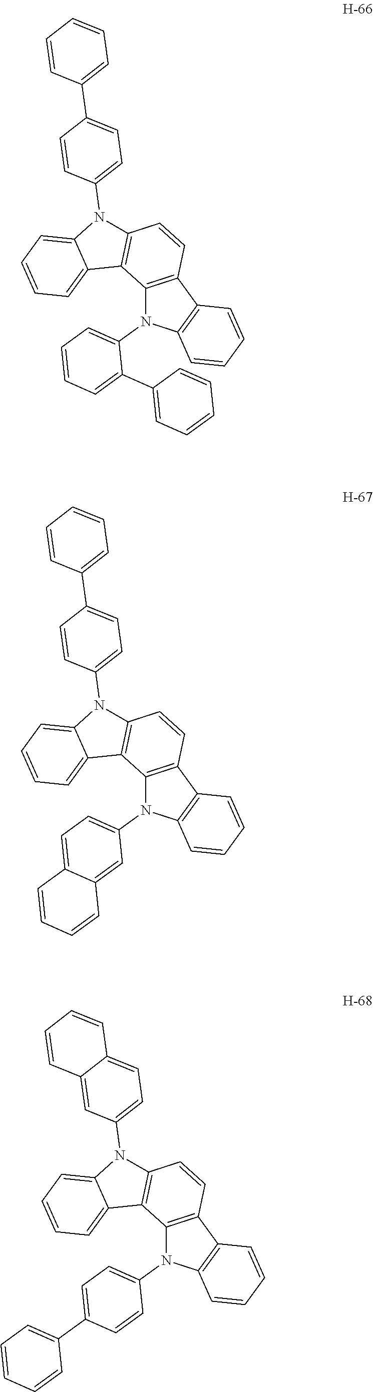 [SMILES: c1ccc(-c2ccc(-n3c4ccccc4c4c3ccc3c5ccccc5n(-c5ccccc5-c5ccccc5)c34)cc2)cc1 ; c1ccc(-c2ccc(-n3c4ccccc4c4c3ccc3c5ccccc5n(-c5ccc6ccccc6c5)c34)cc2)cc1 ; c1ccc(-c2ccc(-n3c4ccccc4c4ccc5c(c6ccccc6n5-c5ccc6ccccc6c5)c43)cc2)cc1]

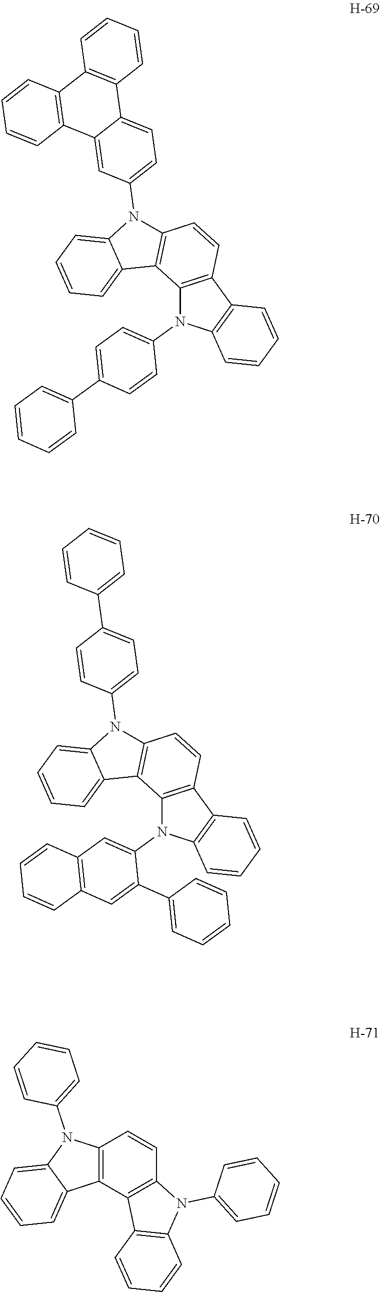 [SMILES: c1ccc(-c2ccc(-n3c4ccccc4c4ccc5c(c6ccccc6n5-c5ccc6c7ccccc7c7ccccc7c6c5)c43)cc2)cc1 ; c1ccc(-c2ccc(-n3c4ccccc4c4c3ccc3c5ccccc5n(-c5cc6ccccc6cc5-c5ccccc5)c34)cc2)cc1 ; c1ccc(-n2c3ccccc3c3c4c5ccccc5n(-c5ccccc5)c4ccc32)cc1]

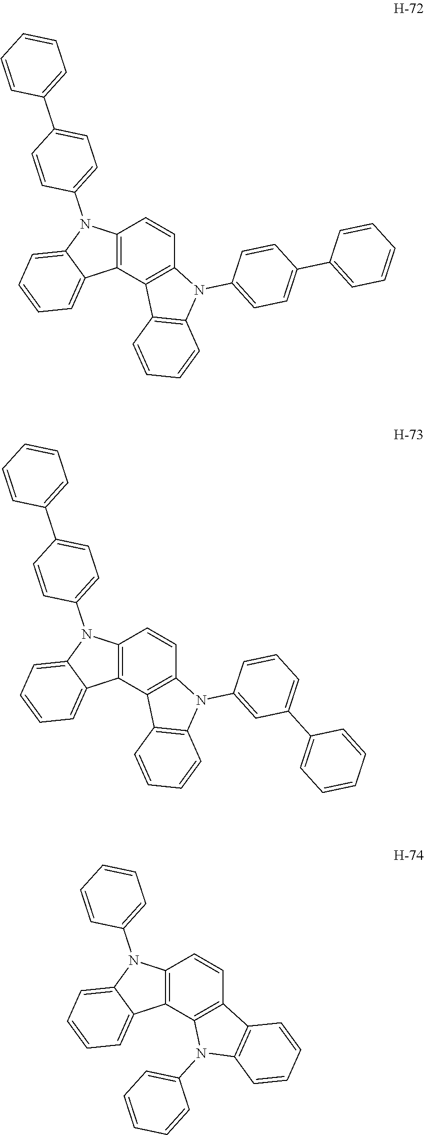 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-n2c3ccccc3c3c2ccc2c4ccccc4n(-c4ccccc4)c23)cc1]

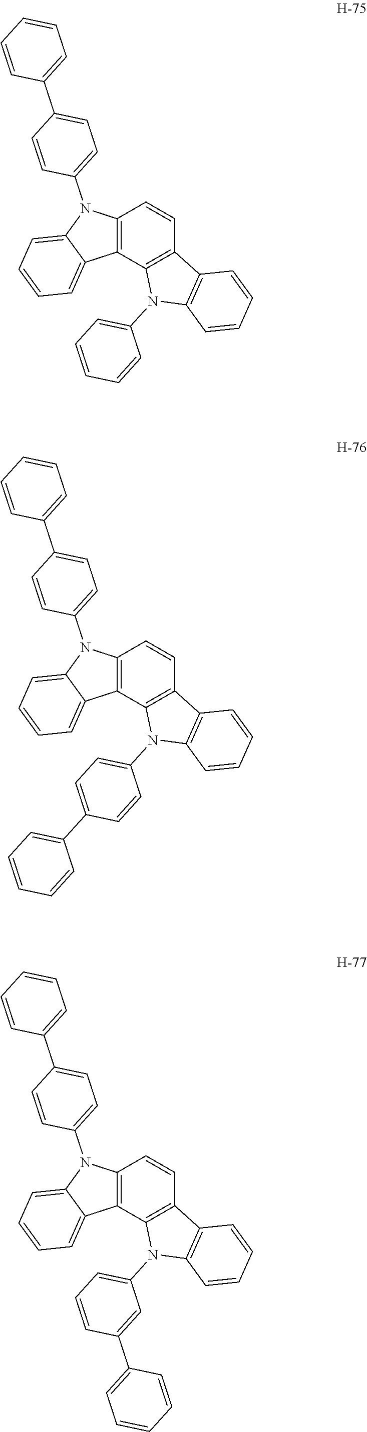 [SMILES: c1ccc(-c2ccc(-n3c4ccccc4c4c3ccc3c5ccccc5n(-c5ccccc5)c34)cc2)cc1 ; c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4c3ccc3c5ccccc5n(-c5cccc(-c6ccccc6)c5)c34)cc2)cc1]

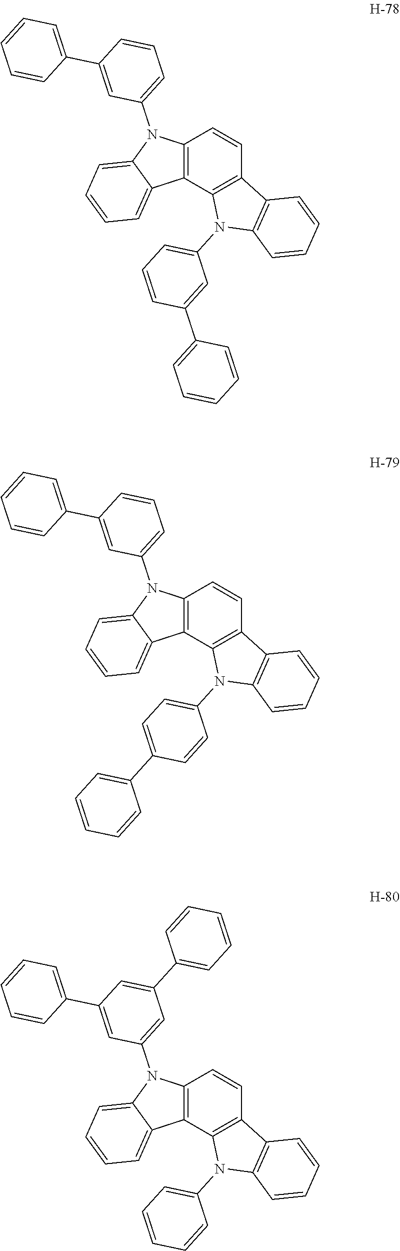 [SMILES: c1ccc(-c2cccc(-n3c4ccccc4c4c3ccc3c5ccccc5n(-c5cccc(-c6ccccc6)c5)c34)c2)cc1 ; c1ccc(-c2ccc(-n3c4ccccc4c4ccc5c(c6ccccc6n5-c5cccc(-c6ccccc6)c5)c43)cc2)cc1 ; c1ccc(-c2cc(-c3ccccc3)cc(-n3c4ccccc4c4c3ccc3c5ccccc5n(-c5ccccc5)c34)c2)cc1]

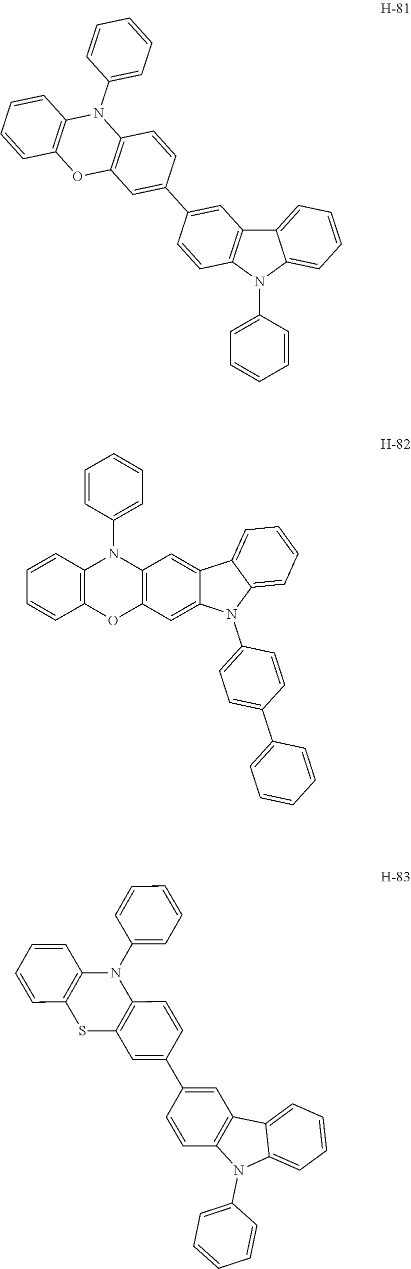 [SMILES: c1ccc(N2c3ccccc3Oc3cc(-c4ccc5c(c4)c4ccccc4n5-c4ccccc4)ccc32)cc1 ; c1ccc(-c2ccc(-n3c4ccccc4c4cc5c(cc43)Oc3ccccc3N5c3ccccc3)cc2)cc1 ; c1ccc(N2c3ccccc3Sc3cc(-c4ccc5c(c4)c4ccccc4n5-c4ccccc4)ccc32)cc1]

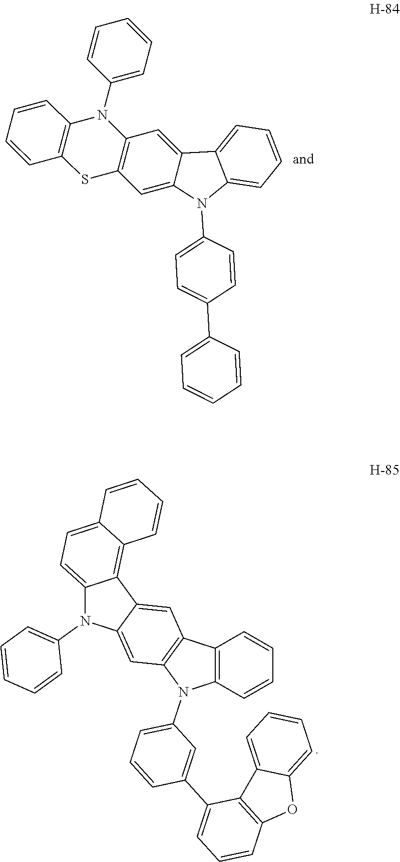 [SMILES: c1ccc(-c2ccc(-n3c4ccccc4c4cc5c(cc43)Sc3ccccc3N5c3ccccc3)cc2)cc1 ; c1ccc(-n2c3cc4c(cc3c3c5ccccc5ccc32)c2ccccc2n4-c2cccc(-c3cccc4oc5ccccc5c34)c2)cc1]

15. An organic electroluminescent device comprising the organic electroluminescent compound according to claim 1.

16. The organic electroluminescent device according to claim 15, wherein the organic electroluminescent compound is included in a hole transport zone and/or an electron transport zone and/or a light-emitting layer.

17. An organic electroluminescent device comprising an anode; a cathode; and at least one light-emitting layer between the anode and the cathode, wherein the at least one light-emitting layer comprises a plurality of host materials according to claim 11.

18. An organic electroluminescent compound represented by the following formula 2: 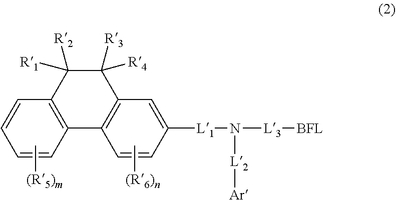
wherein
R′₁ to R′₄ each independently represent hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent(s) to form a ring(s);
R′₅ and R′₆ each independently represent hydrogen or deuterium;
L′₁ to L′₃ each independently represent a single bond, a substituted or unsubstituted (C6-C30)arylene, or a substituted or unsubstituted (3- to 30-membered)heteroarylene;
Ar′ represents a substituted or unsubstituted (C6-C30)aryl, or a substituted or unsubstituted (3- to 30-membered)heteroaryl;
BFL represents a substituted or unsubstituted benzo[a]fluorenyl, a substituted or unsubstituted benzo[b]fluorenyl, or a substituted or unsubstituted benzo[c]fluorenyl; and
m represent an integer of 1 to 4, n represents an integer of 1 to 3, and when m and n are 2 or more, each of R′₅ and each of R′₆ may be the same or different.

19. The organic electroluminescent compound according to claim 18, wherein the compound represented by formula 2 is selected from the following compounds:

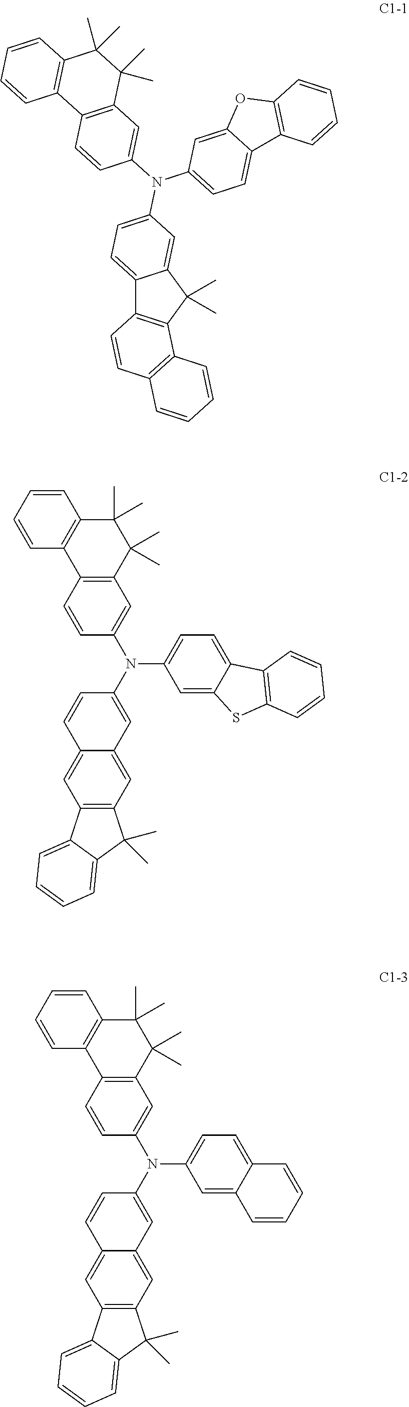 [SMILES: CC1(C)c2cc(N(c3ccc4c(c3)C(C)(C)C(C)(C)c3ccccc3-4)c3ccc4c(c3)oc3ccccc34)ccc2-c2ccc3ccccc3c21 ; CC1(C)c2ccccc2-c2cc3ccc(N(c4ccc5c(c4)C(C)(C)C(C)(C)c4ccccc4-5)c4ccc5c(c4)sc4ccccc45)cc3cc21 ; CC1(C)c2ccccc2-c2cc3ccc(N(c4ccc5c(c4)C(C)(C)C(C)(C)c4ccccc4-5)c4ccc5ccccc5c4)cc3cc21]

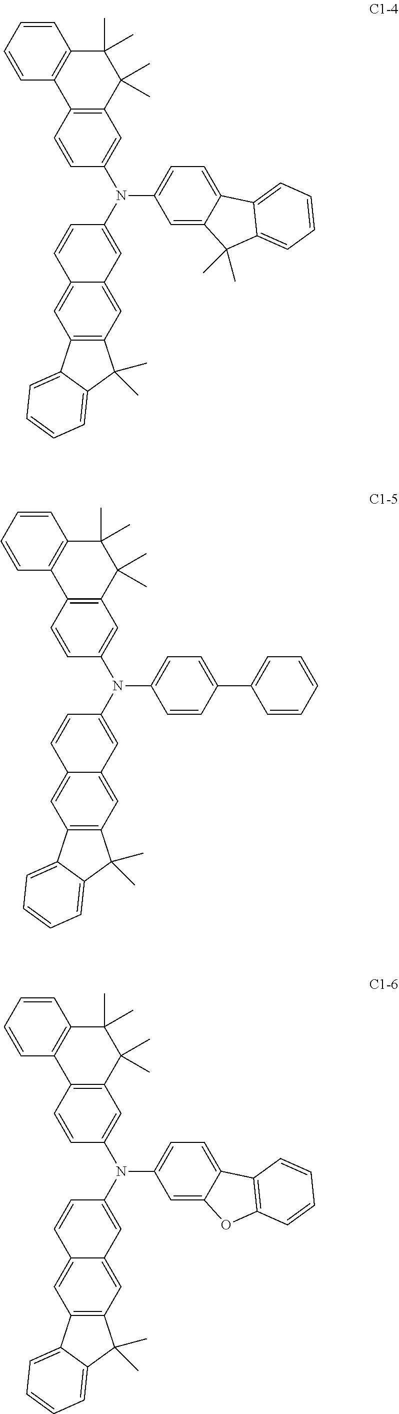 [SMILES: CC1(C)c2ccccc2-c2ccc(N(c3ccc4c(c3)C(C)(C)C(C)(C)c3ccccc3-4)c3ccc4cc5c(cc4c3)C(C)(C)c3ccccc3-5)cc21 ; CC1(C)c2ccccc2-c2cc3ccc(N(c4ccc(-c5ccccc5)cc4)c4ccc5c(c4)C(C)(C)C(C)(C)c4ccccc4-5)cc3cc21 ; CC1(C)c2ccccc2-c2cc3ccc(N(c4ccc5c(c4)C(C)(C)C(C)(C)c4ccccc4-5)c4ccc5c(c4)oc4ccccc45)cc3cc21]

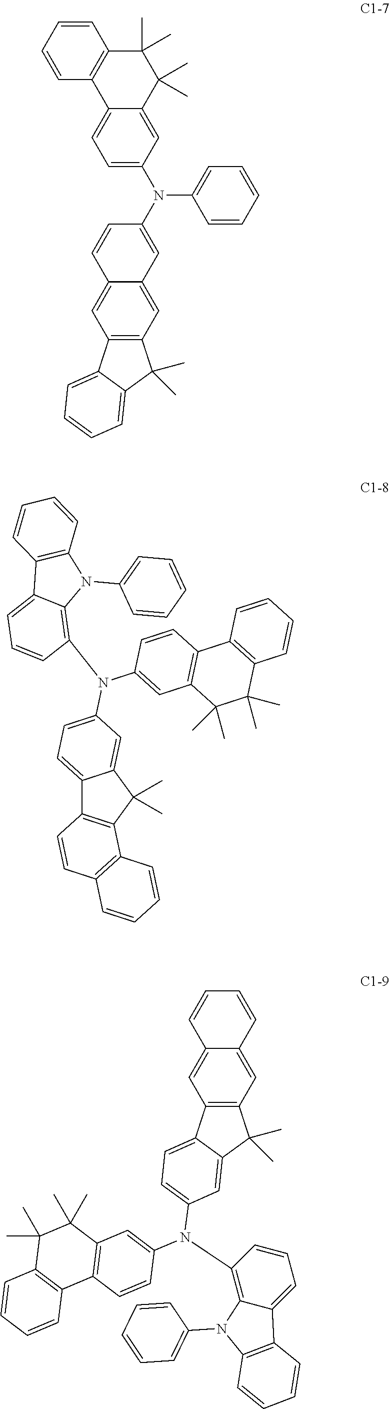 [SMILES: CC1(C)c2ccccc2-c2cc3ccc(N(c4ccccc4)c4ccc5c(c4)C(C)(C)C(C)(C)c4ccccc4-5)cc3cc21 ; CC1(C)c2cc(N(c3ccc4c(c3)C(C)(C)C(C)(C)c3ccccc3-4)c3cccc4c5ccccc5n(-c5ccccc5)c34)ccc2-c2ccc3ccccc3c21 ; CC1(C)c2cc(N(c3ccc4c(c3)C(C)(C)C(C)(C)c3ccccc3-4)c3cccc4c5ccccc5n(-c5ccccc5)c34)ccc2-c2cc3ccccc3cc21]

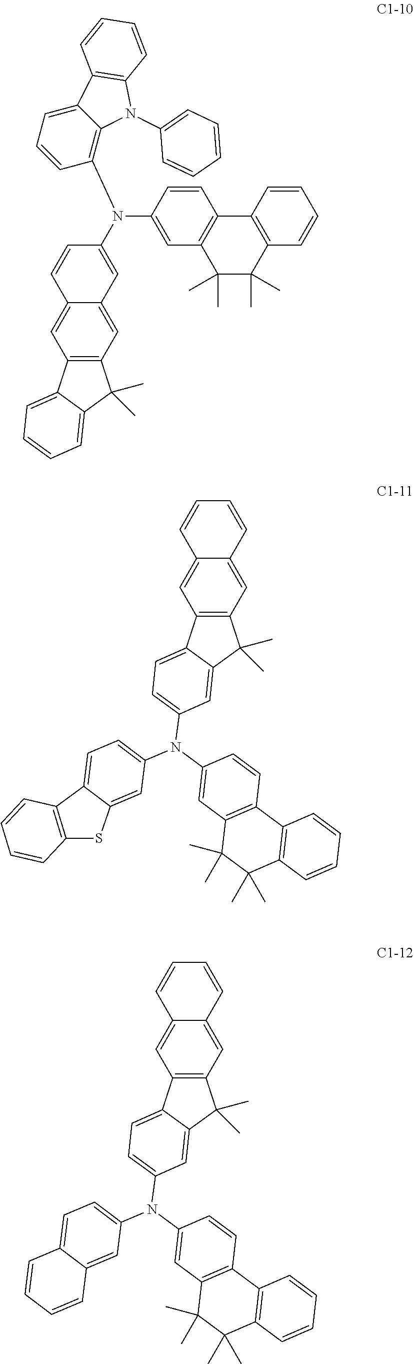 [SMILES: CC1(C)c2ccccc2-c2cc3ccc(N(c4ccc5c(c4)C(C)(C)C(C)(C)c4ccccc4-5)c4cccc5c6ccccc6n(-c6ccccc6)c45)cc3cc21 ; CC1(C)c2cc(N(c3ccc4c(c3)C(C)(C)C(C)(C)c3ccccc3-4)c3ccc4c(c3)sc3ccccc34)ccc2-c2cc3ccccc3cc21 ; CC1(C)c2cc(N(c3ccc4c(c3)C(C)(C)C(C)(C)c3ccccc3-4)c3ccc4ccccc4c3)ccc2-c2cc3ccccc3cc21]

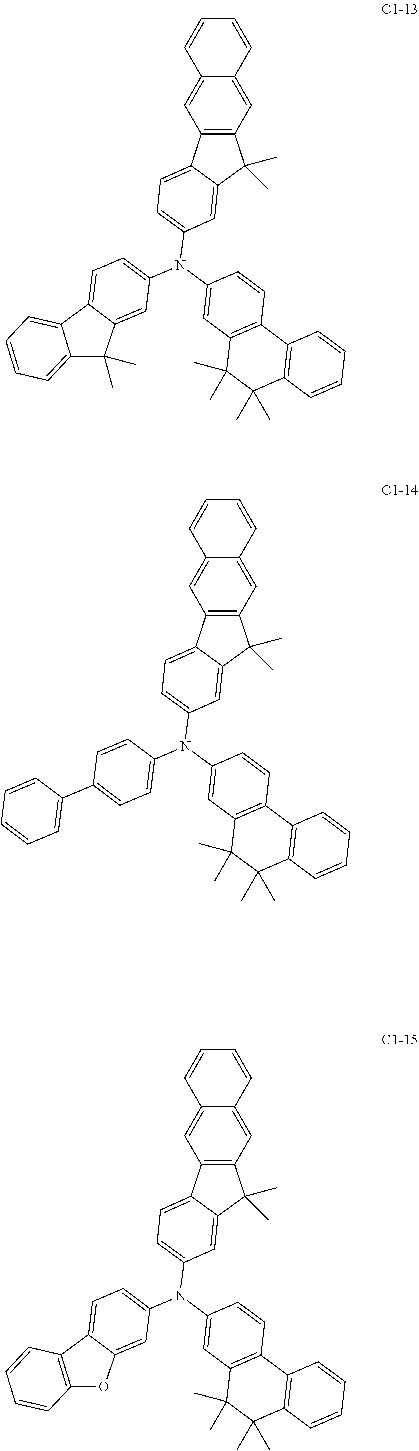 [SMILES: CC1(C)c2ccccc2-c2ccc(N(c3ccc4c(c3)C(C)(C)c3cc5ccccc5cc3-4)c3ccc4c(c3)C(C)(C)C(C)(C)c3ccccc3-4)cc21 ; CC1(C)c2cc(N(c3ccc(-c4ccccc4)cc3)c3ccc4c(c3)C(C)(C)C(C)(C)c3ccccc3-4)ccc2-c2cc3ccccc3cc21 ; CC1(C)c2cc(N(c3ccc4c(c3)C(C)(C)C(C)(C)c3ccccc3-4)c3ccc4c(c3)oc3ccccc34)ccc2-c2cc3ccccc3cc21]

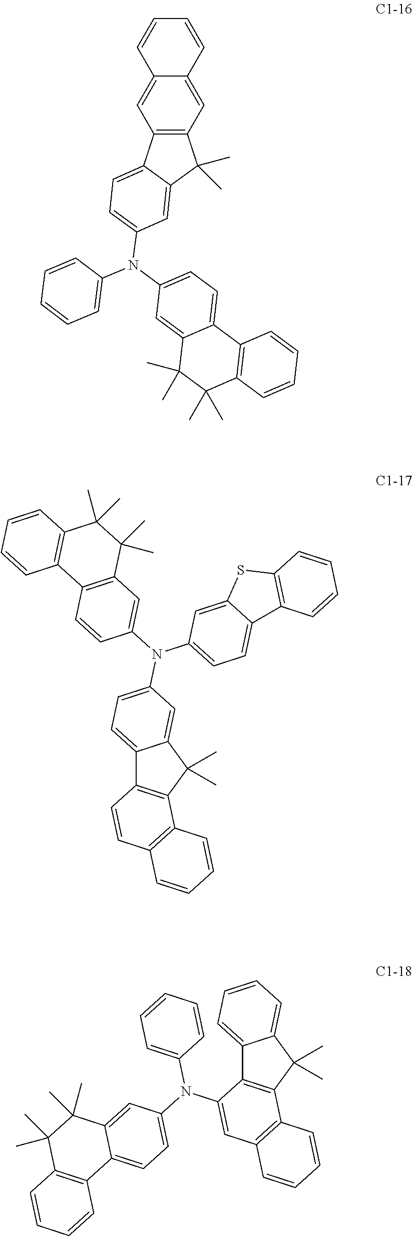

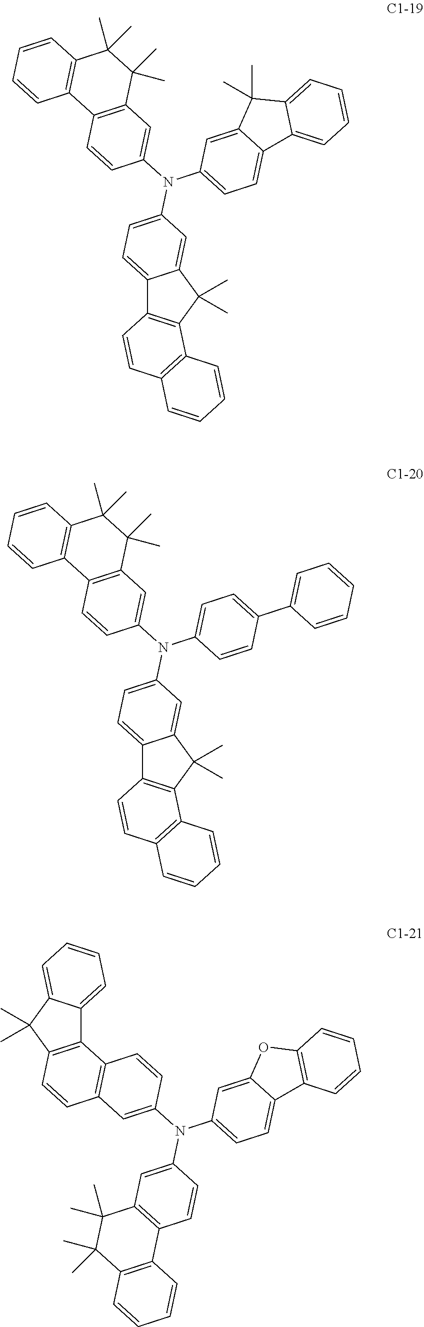 [SMILES: CC1(C)c2ccccc2-c2ccc(N(c3ccc4c(c3)C(C)(C)c3c-4ccc4ccccc34)c3ccc4c(c3)C(C)(C)C(C)(C)c3ccccc3-4)cc21 ; CC1(C)c2cc(N(c3ccc(-c4ccccc4)cc3)c3ccc4c(c3)C(C)(C)C(C)(C)c3ccccc3-4)ccc2-c2ccc3ccccc3c21 ; CC1(C)c2ccccc2-c2c1ccc1cc(N(c3ccc4c(c3)C(C)(C)C(C)(C)c3ccccc3-4)c3ccc4c(c3)oc3ccccc34)ccc21]

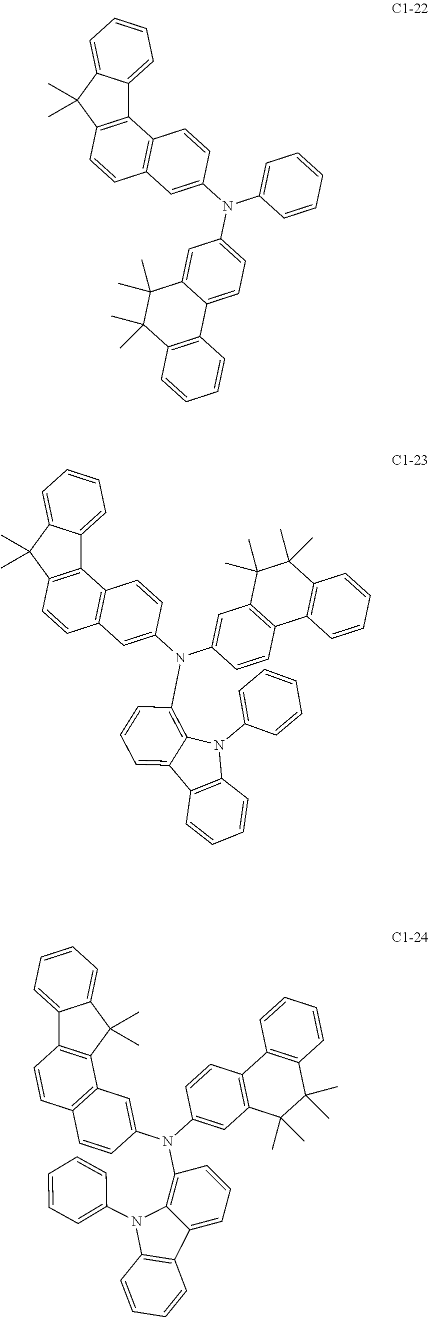 [SMILES: CC1(C)c2ccccc2-c2c1ccc1cc(N(c3ccccc3)c3ccc4c(c3)C(C)(C)C(C)(C)c3ccccc3-4)ccc21 ; CC1(C)c2ccccc2-c2c1ccc1cc(N(c3ccc4c(c3)C(C)(C)C(C)(C)c3ccccc3-4)c3cccc4c5ccccc5n(-c5ccccc5)c34)ccc21 ; CC1(C)c2ccccc2-c2ccc3ccc(N(c4ccc5c(c4)C(C)(C)C(C)(C)c4ccccc4-5)c4cccc5c6ccccc6n(-c6ccccc6)c45)cc3c21]

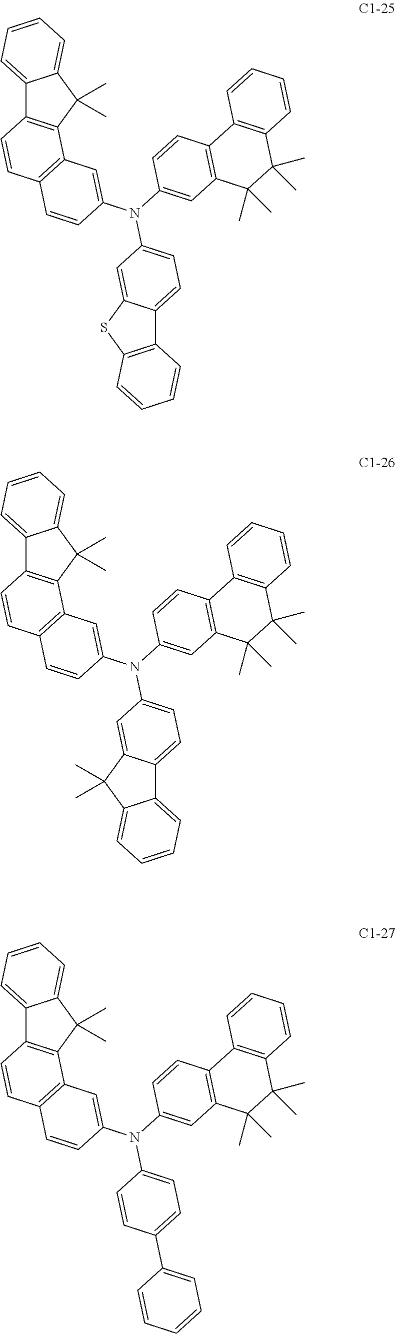 [SMILES: CC1(C)c2ccccc2-c2ccc3ccc(N(c4ccc5c(c4)C(C)(C)C(C)(C)c4ccccc4-5)c4ccc5c(c4)sc4ccccc45)cc3c21 ; CC1(C)c2ccccc2-c2ccc(N(c3ccc4c(c3)C(C)(C)C(C)(C)c3ccccc3-4)c3ccc4ccc5c(c4c3)C(C)(C)c3ccccc3-5)cc21 ; CC1(C)c2ccccc2-c2ccc3ccc(N(c4ccc(-c5ccccc5)cc4)c4ccc5c(c4)C(C)(C)C(C)(C)c4ccccc4-5)cc3c21]

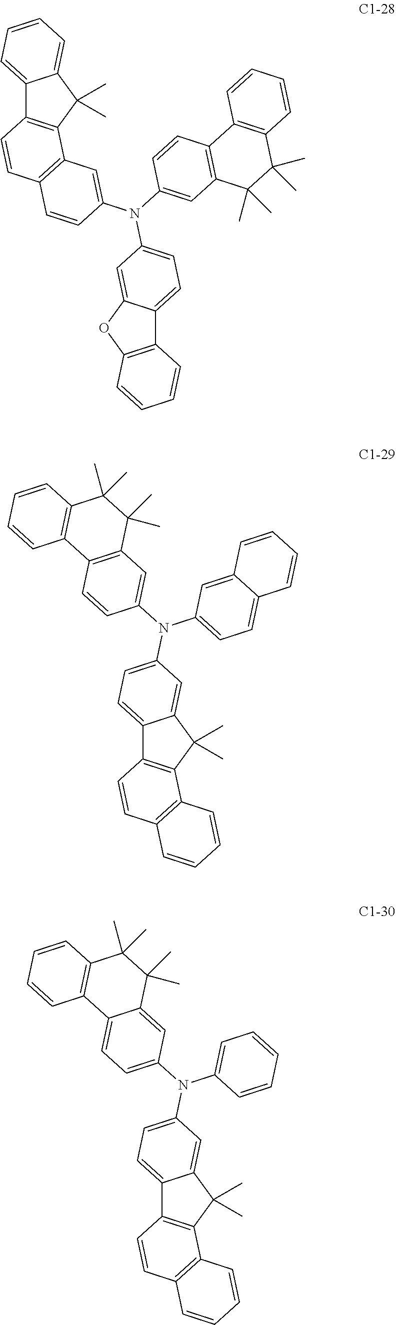 [SMILES: CC1(C)c2ccccc2-c2ccc3ccc(N(c4ccc5c(c4)C(C)(C)C(C)(C)c4ccccc4-5)c4ccc5c(c4)oc4ccccc45)cc3c21 ; CC1(C)c2cc(N(c3ccc4c(c3)C(C)(C)C(C)(C)c3ccccc3-4)c3ccc4ccccc4c3)ccc2-c2ccc3ccccc3c21 ; CC1(C)c2cc(N(c3ccccc3)c3ccc4c(c3)C(C)(C)C(C)(C)c3ccccc3-4)ccc2-c2ccc3ccccc3c21]

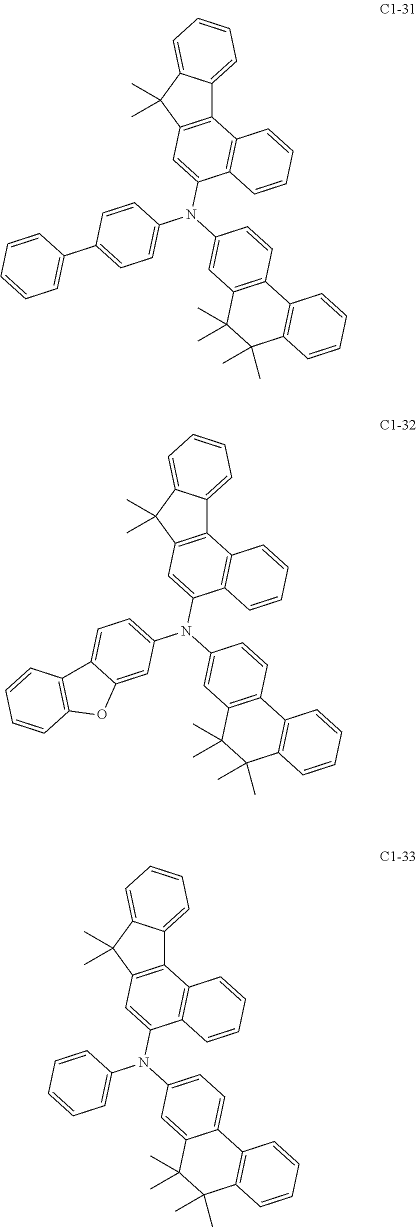 [SMILES: CC1(C)c2ccccc2-c2c1cc(N(c1ccc(-c3ccccc3)cc1)c1ccc3c(c1)C(C)(C)C(C)(C)c1ccccc1-3)c1ccccc21 ; CC1(C)c2ccccc2-c2c1cc(N(c1ccc3c(c1)C(C)(C)C(C)(C)c1ccccc1-3)c1ccc3c(c1)oc1ccccc13)c1ccccc21 ; CC1(C)c2ccccc2-c2c1cc(N(c1ccccc1)c1ccc3c(c1)C(C)(C)C(C)(C)c1ccccc1-3)c1ccccc21]

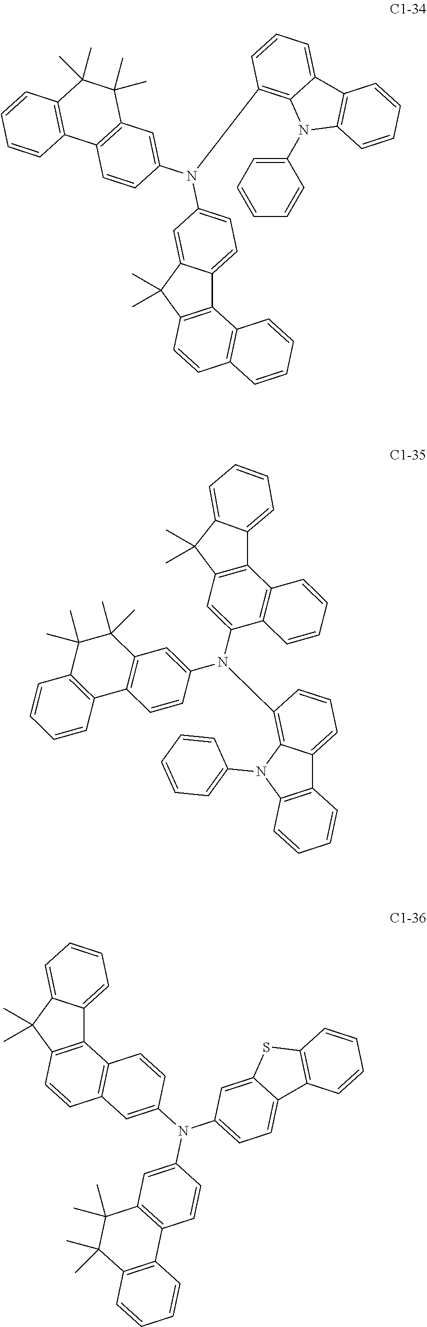 [SMILES: CC1(C)c2cc(N(c3ccc4c(c3)C(C)(C)C(C)(C)c3ccccc3-4)c3cccc4c5ccccc5n(-c5ccccc5)c34)ccc2-c2c1ccc1ccccc21 ; CC1(C)c2ccccc2-c2c1cc(N(c1ccc3c(c1)C(C)(C)C(C)(C)c1ccccc1-3)c1cccc3c4ccccc4n(-c4ccccc4)c13)c1ccccc21 ; CC1(C)c2ccccc2-c2c1ccc1cc(N(c3ccc4c(c3)C(C)(C)C(C)(C)c3ccccc3-4)c3ccc4c(c3)sc3ccccc34)ccc21]

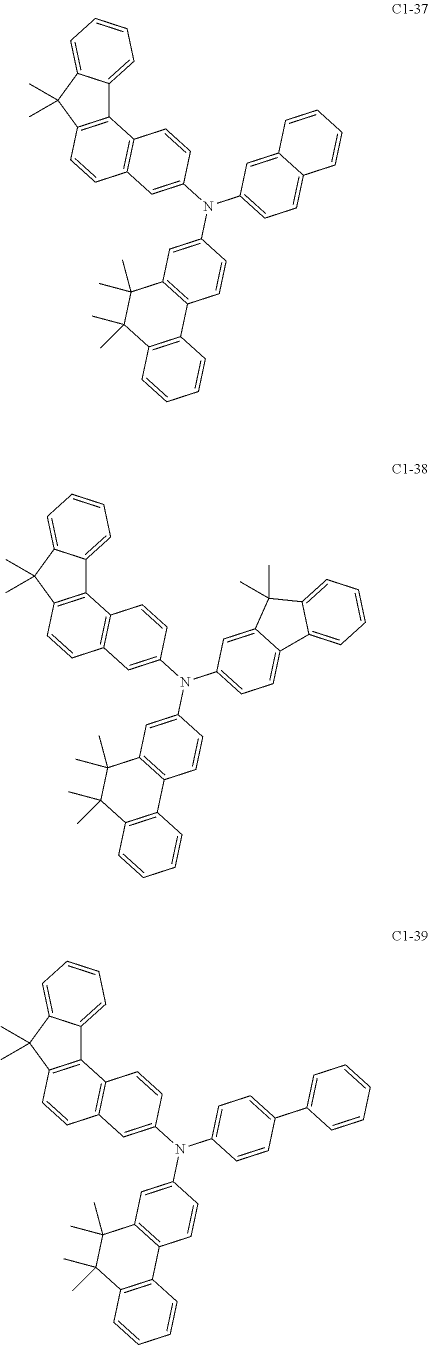 [SMILES: CC1(C)c2ccccc2-c2c1ccc1cc(N(c3ccc4c(c3)C(C)(C)C(C)(C)c3ccccc3-4)c3ccc4ccccc4c3)ccc21 ; CC1(C)c2ccccc2-c2ccc(N(c3ccc4c(c3)C(C)(C)C(C)(C)c3ccccc3-4)c3ccc4c5c(ccc4c3)C(C)(C)c3ccccc3-5)cc21 ; CC1(C)c2ccccc2-c2c1ccc1cc(N(c3ccc(-c4ccccc4)cc3)c3ccc4c(c3)C(C)(C)C(C)(C)c3ccccc3-4)ccc21]

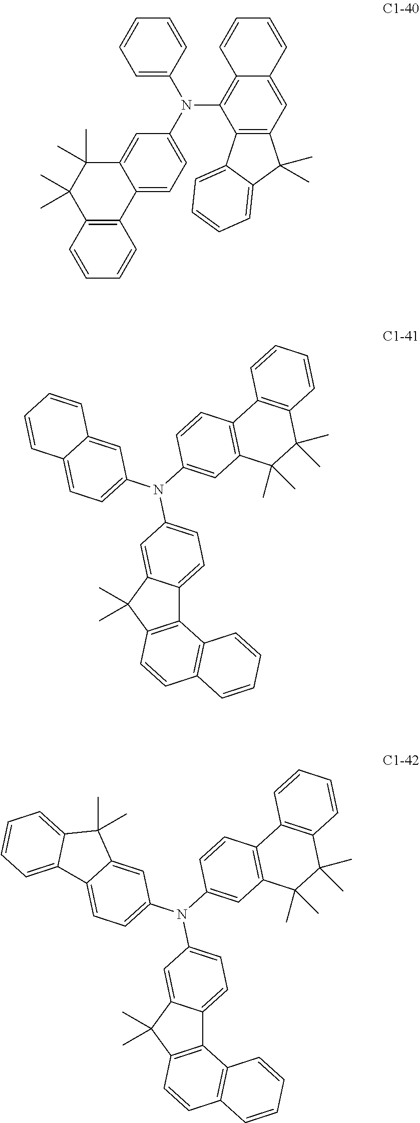

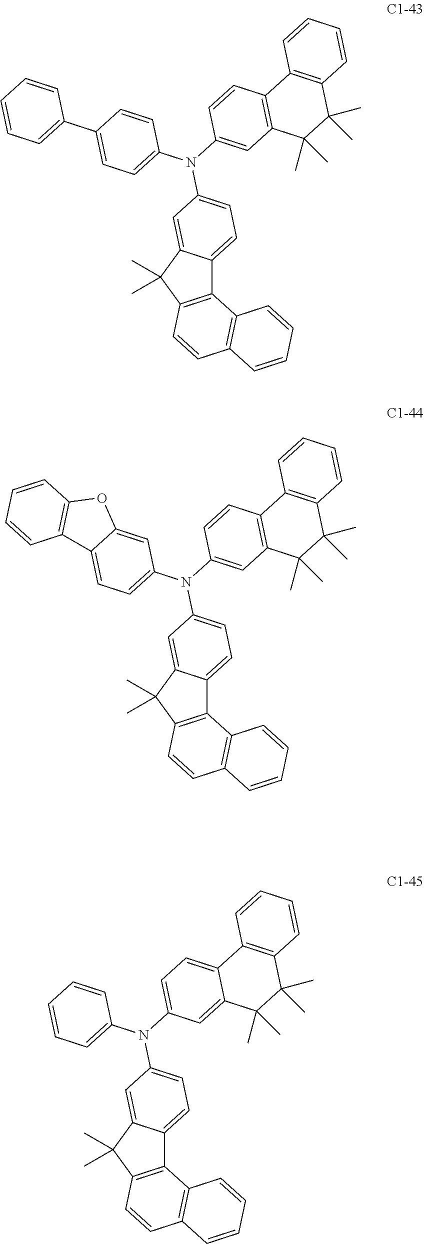 [SMILES: CC1(C)c2cc(N(c3ccc(-c4ccccc4)cc3)c3ccc4c(c3)C(C)(C)C(C)(C)c3ccccc3-4)ccc2-c2c1ccc1ccccc21 ; CC1(C)c2cc(N(c3ccc4c(c3)C(C)(C)C(C)(C)c3ccccc3-4)c3ccc4c(c3)oc3ccccc34)ccc2-c2c1ccc1ccccc21 ; CC1(C)c2cc(N(c3ccccc3)c3ccc4c(c3)C(C)(C)C(C)(C)c3ccccc3-4)ccc2-c2c1ccc1ccccc21]

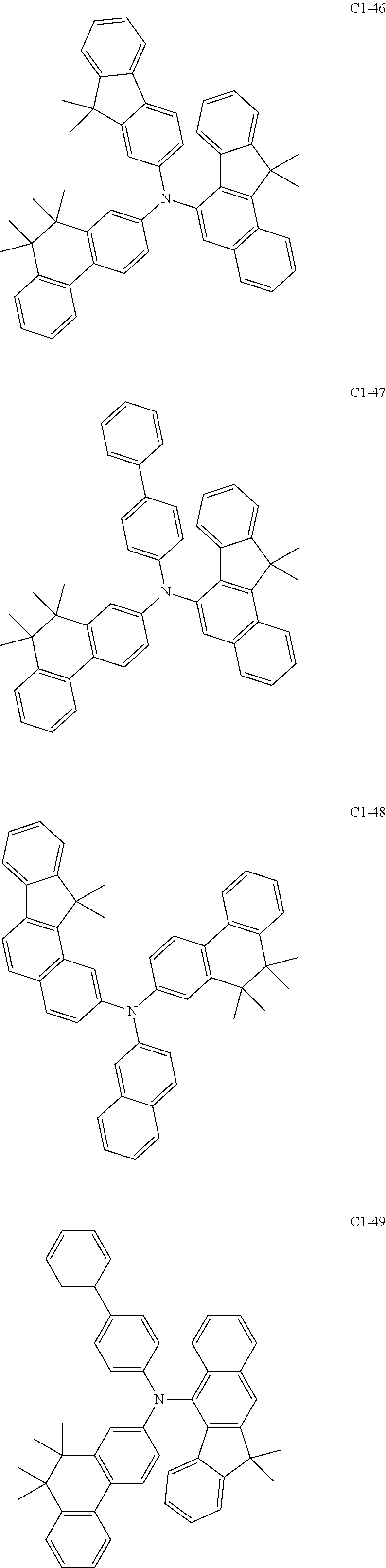

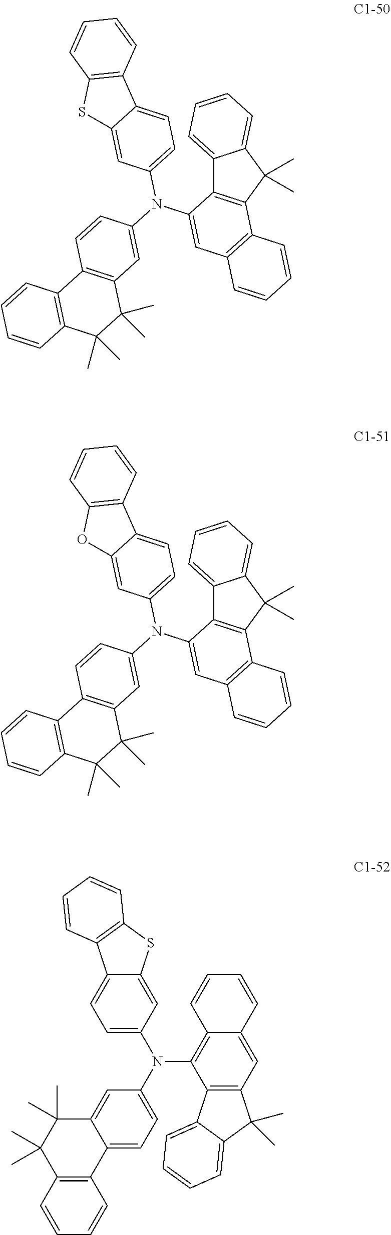

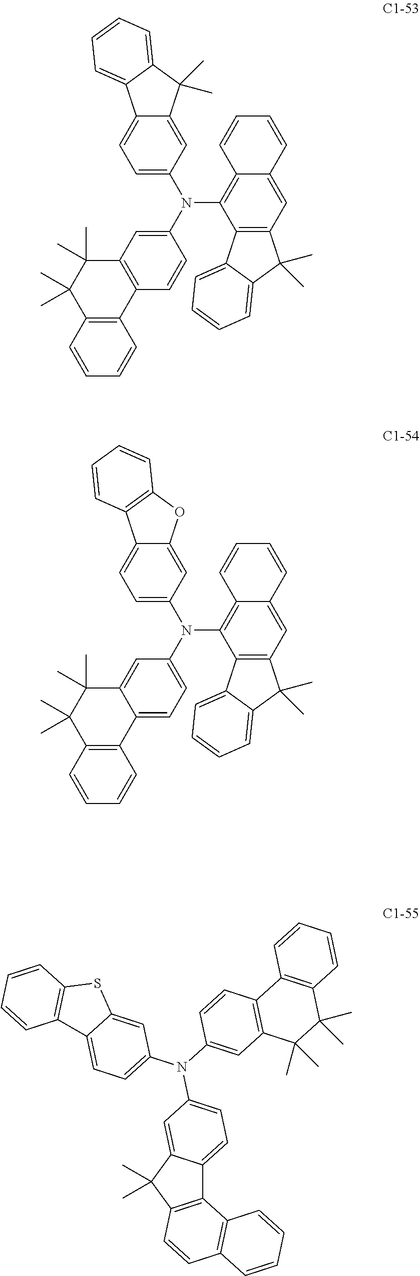

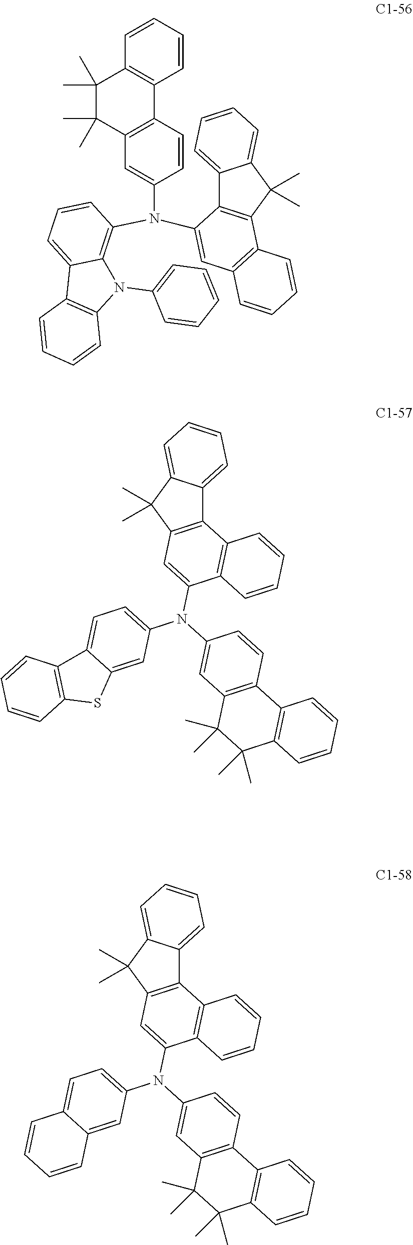 [SMILES: CC1(C)c2ccccc2-c2c(N(c3ccc4c(c3)C(C)(C)C(C)(C)c3ccccc3-4)c3cccc4c5ccccc5n(-c5ccccc5)c34)cc3ccccc3c21 ; CC1(C)c2ccccc2-c2c1cc(N(c1ccc3c(c1)C(C)(C)C(C)(C)c1ccccc1-3)c1ccc3c(c1)sc1ccccc13)c1ccccc21 ; CC1(C)c2ccccc2-c2c1cc(N(c1ccc3c(c1)C(C)(C)C(C)(C)c1ccccc1-3)c1ccc3ccccc3c1)c1ccccc21]

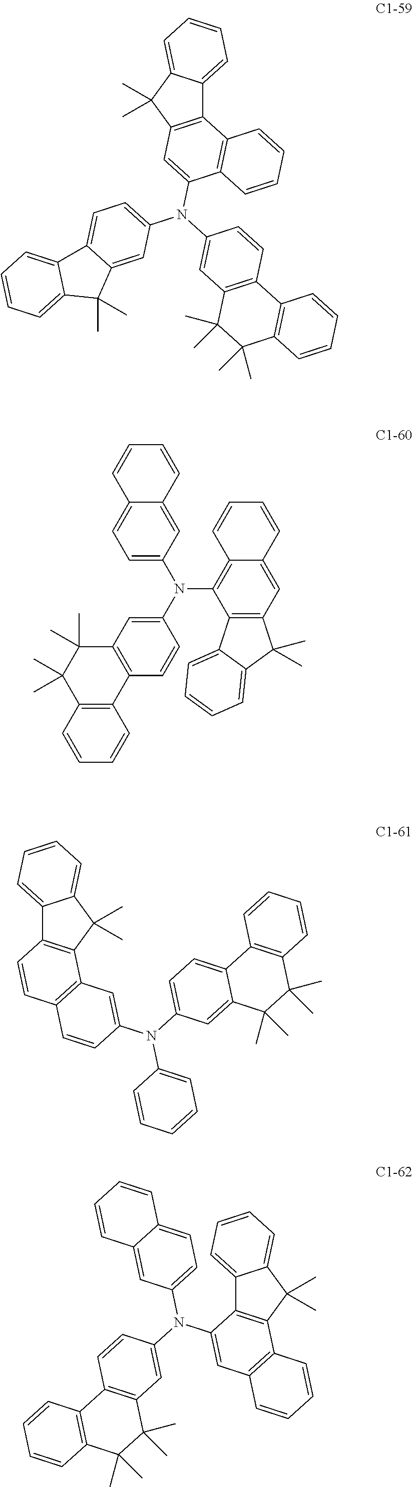

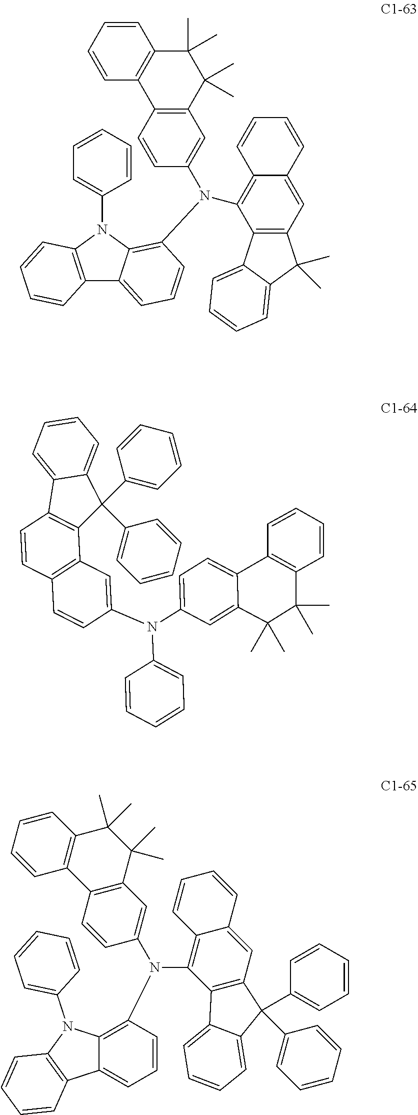 [SMILES: CC1(C)c2ccccc2-c2c1cc1ccccc1c2N(c1ccc2c(c1)C(C)(C)C(C)(C)c1ccccc1-2)c1cccc2c3ccccc3n(-c3ccccc3)c12 ; CC1(C)c2ccccc2-c2ccc(N(c3ccccc3)c3ccc4ccc5c(c4c3)C(c3ccccc3)(c3ccccc3)c3ccccc3-5)cc2C1(C)C ; CC1(C)c2ccccc2-c2ccc(N(c3c4c(cc5ccccc35)C(c3ccccc3)(c3ccccc3)c3ccccc3-4)c3cccc4c5ccccc5n(-c5ccccc5)c34)cc2C1(C)C]

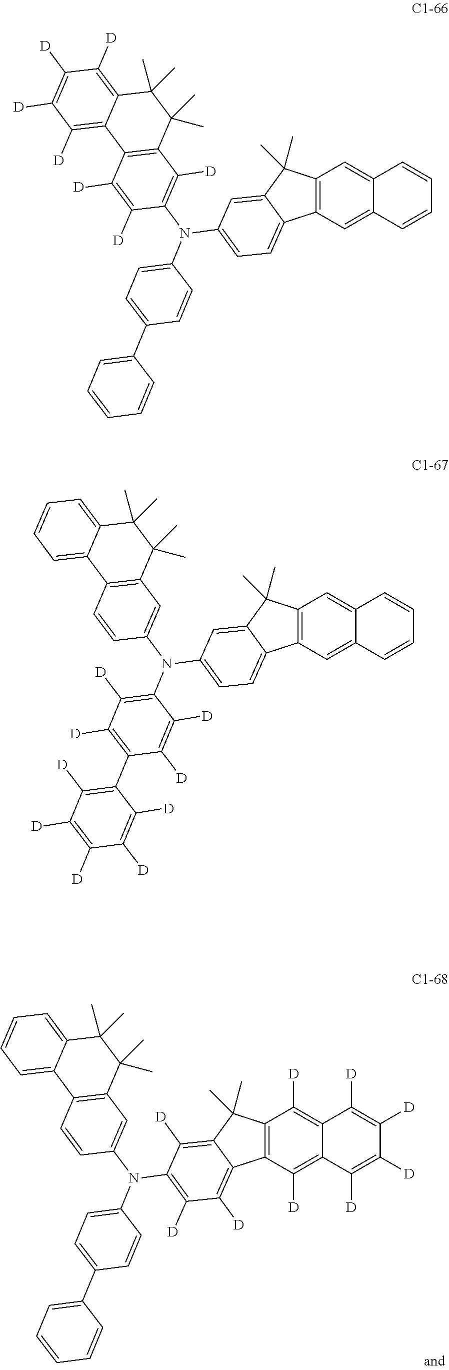 [SMILES: [2H]c1c([2H])c([2H])c2c(c1[2H])-c1c([2H])c([2H])c(N(c3ccc(-c4ccccc4)cc3)c3ccc4c(c3)C(C)(C)c3cc5ccccc5cc3-4)c([2H])c1C(C)(C)C2(C)C ; [2H]c1c([2H])c([2H])c(-c2c([2H])c([2H])c(N(c3ccc4c(c3)C(C)(C)c3cc5ccccc5cc3-4)c3ccc4c(c3)C(C)(C)C(C)(C)c3ccccc3-4)c([2H])c2[2H])c([2H])c1[2H] ; [2H]c1c([2H])c(N(c2ccc(-c3ccccc3)cc2)c2ccc3c(c2)C(C)(C)C(C)(C)c2ccccc2-3)c([2H])c2c1-c1c(c([2H])c3c([2H])c([2H])c([2H])c([2H])c3c1[2H])C2(C)C]

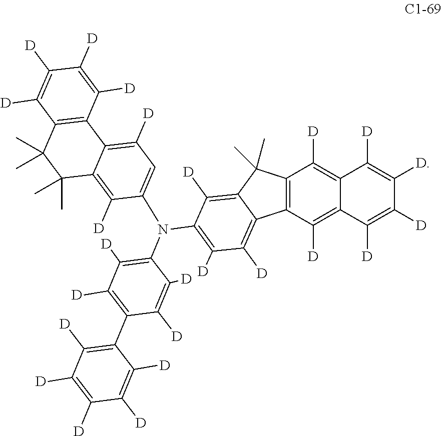

20. An organic electroluminescent device comprising the organic electroluminescent compound according to claim 18.

21. An organic electroluminescent compound represented by the following formula 3: 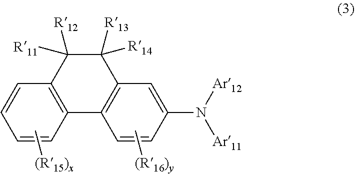
wherein
R′₁₁ to R′₁₄ each independently represent substituted or unsubstituted methyl;
R′₁₅ and R′₁₆ each independently represent hydrogen or deuterium;
Ar′₁₁ and Ar′₁₂ each independently represent phenyl unsubstituted or substituted with deuterium, biphenyl unsubstituted or substituted with deuterium, terphenyl unsubstituted or substituted with deuterium, naphthyl unsubstituted or substituted with deuterium, a group of the following formula (a) unsubstituted or substituted with deuterium, or a combination thereof: 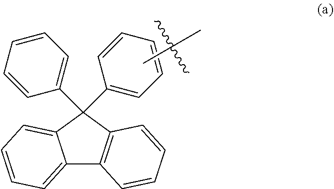
x represents an integer of 1 to 4, y represents an integer of 1 to 3, and when x and y are 2 or more, each of R′₁₅ and each of R′₁₆ may be the same or different.

22. The organic electroluminescent compound according to claim 21, wherein the compound represented by formula 3 is selected from the following compounds: 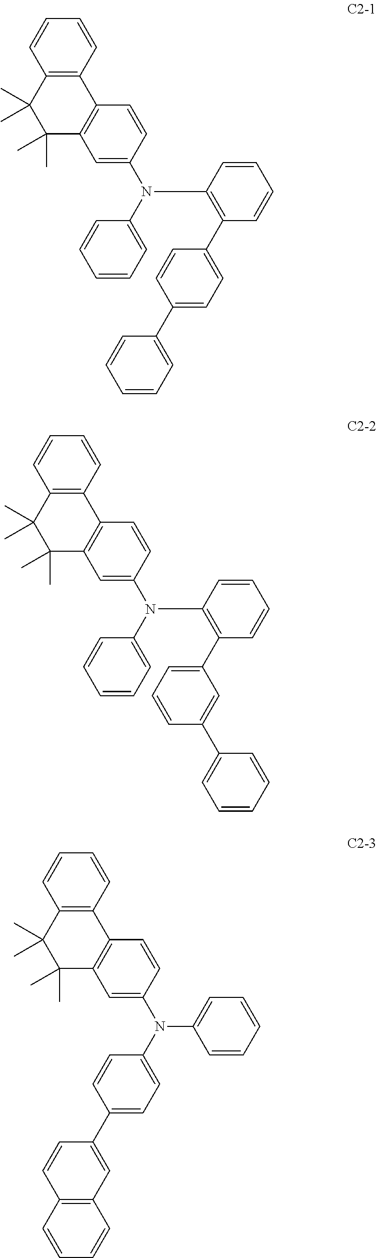 [SMILES: CC1(C)c2ccccc2-c2ccc(N(c3ccccc3)c3ccccc3-c3ccc(-c4ccccc4)cc3)cc2C1(C)C ; CC1(C)c2ccccc2-c2ccc(N(c3ccccc3)c3ccccc3-c3cccc(-c4ccccc4)c3)cc2C1(C)C ; CC1(C)c2ccccc2-c2ccc(N(c3ccccc3)c3ccc(-c4ccc5ccccc5c4)cc3)cc2C1(C)C] 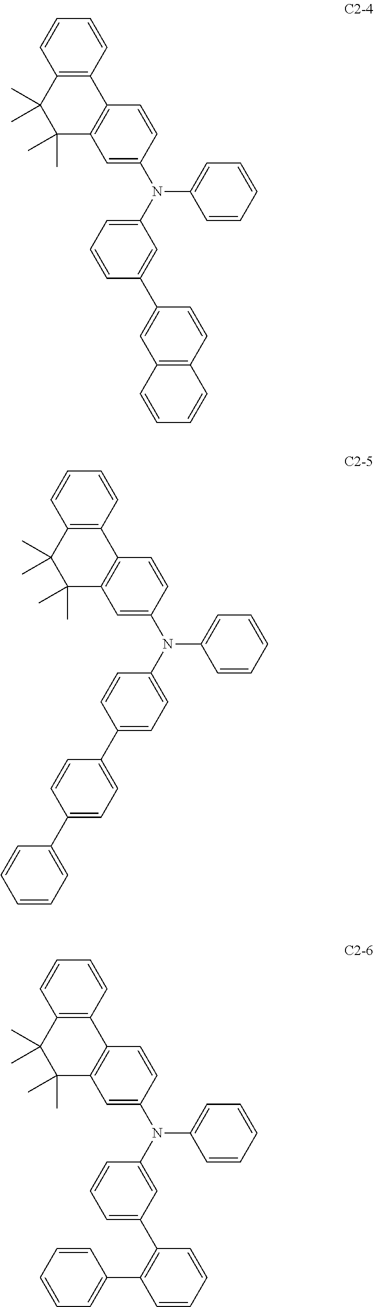 [SMILES: CC1(C)c2ccccc2-c2ccc(N(c3ccccc3)c3cccc(-c4ccc5ccccc5c4)c3)cc2C1(C)C ; CC1(C)c2ccccc2-c2ccc(N(c3ccccc3)c3ccc(-c4ccc(-c5ccccc5)cc4)cc3)cc2C1(C)C ; CC1(C)c2ccccc2-c2ccc(N(c3ccccc3)c3cccc(-c4ccccc4-c4ccccc4)c3)cc2C1(C)C] 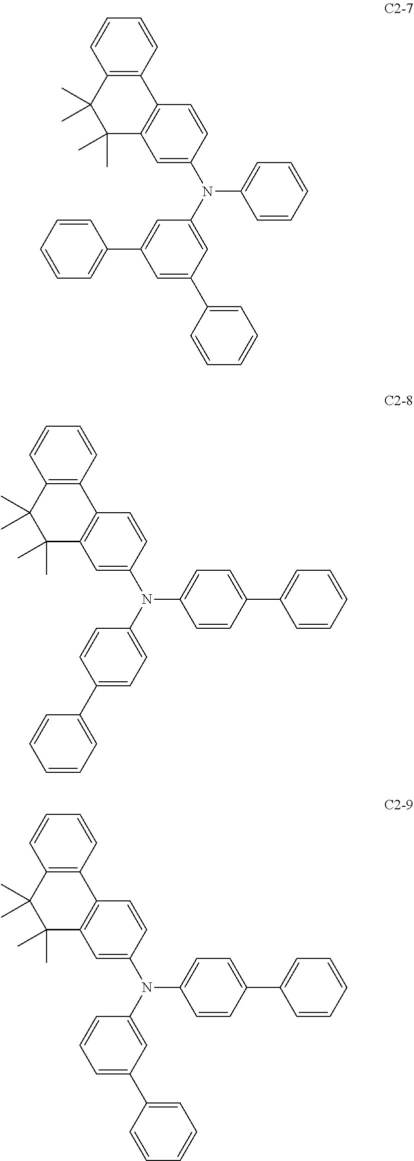 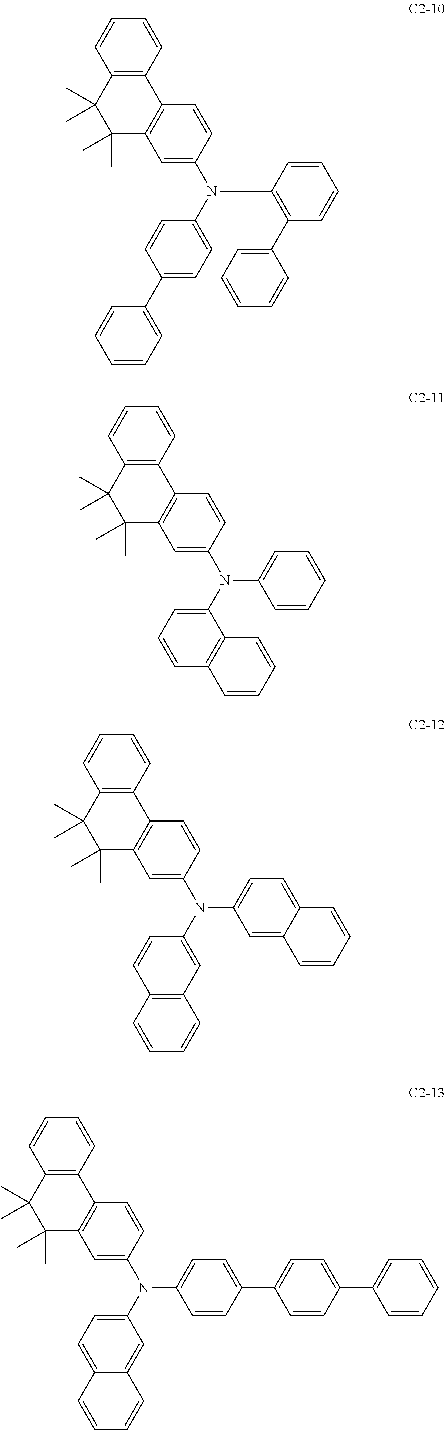 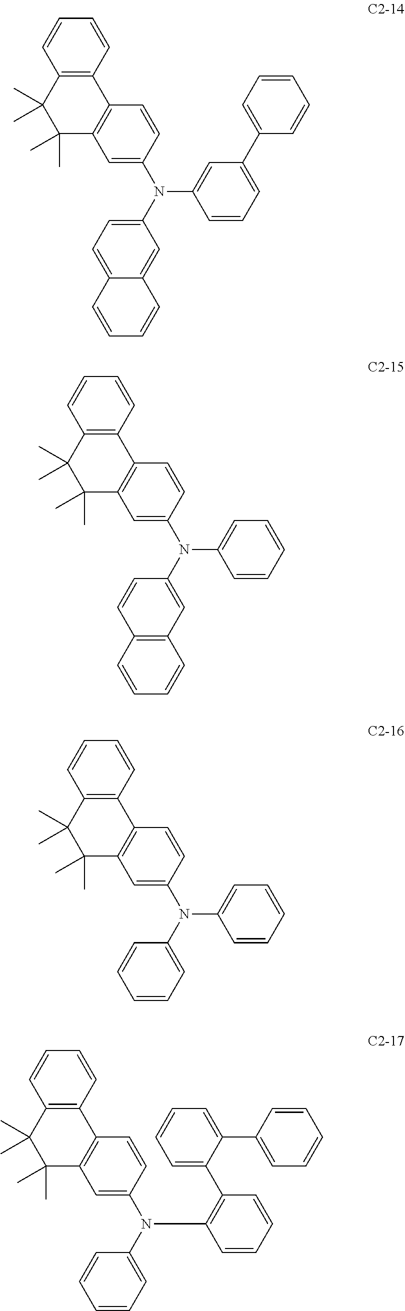 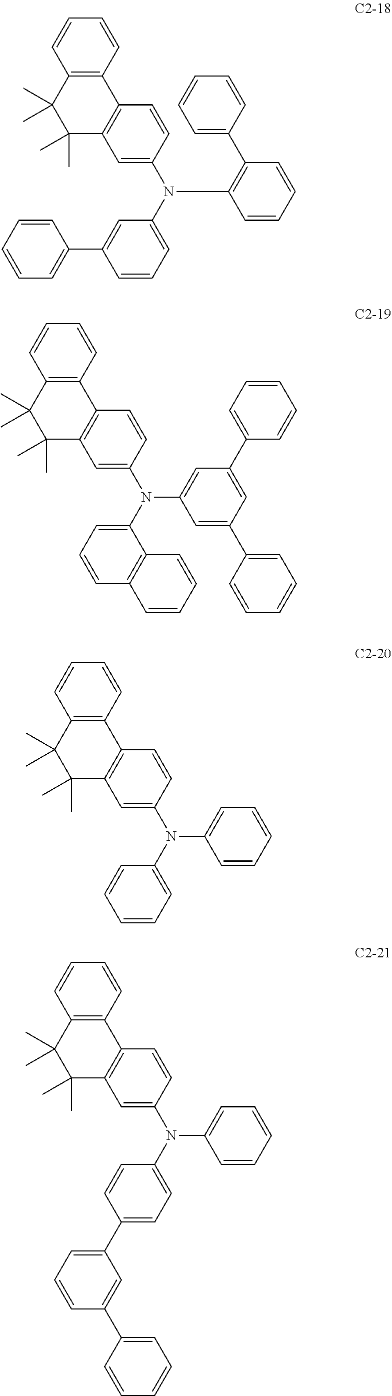 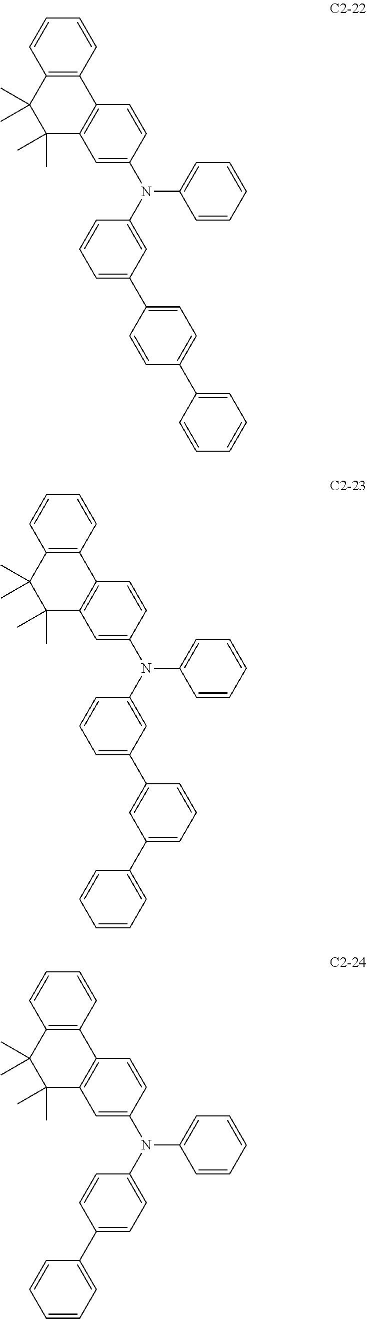 [SMILES: CC1(C)c2ccccc2-c2ccc(N(c3ccccc3)c3cccc(-c4ccc(-c5ccccc5)cc4)c3)cc2C1(C)C ; CC1(C)c2ccccc2-c2ccc(N(c3ccccc3)c3cccc(-c4cccc(-c5ccccc5)c4)c3)cc2C1(C)C ; CC1(C)c2ccccc2-c2ccc(N(c3ccccc3)c3ccc(-c4ccccc4)cc3)cc2C1(C)C] 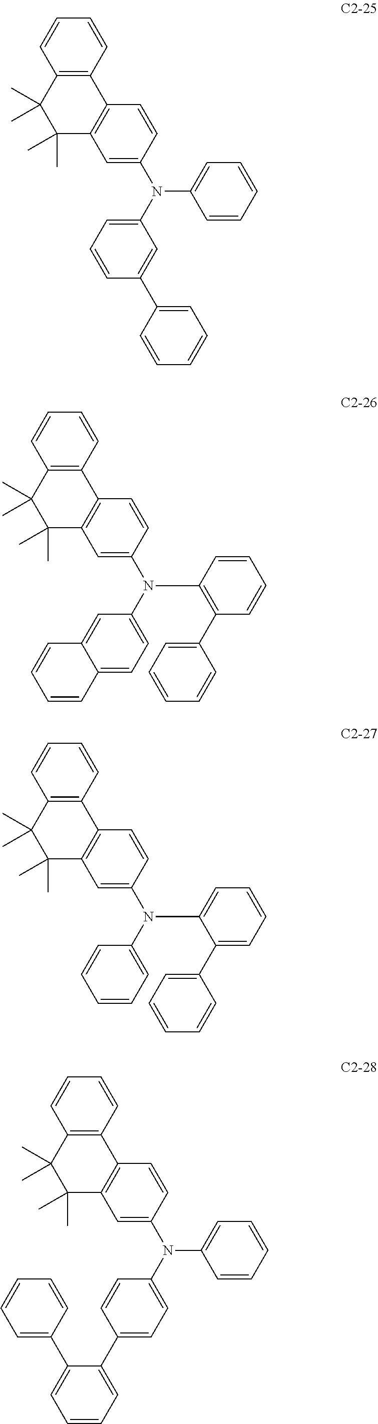 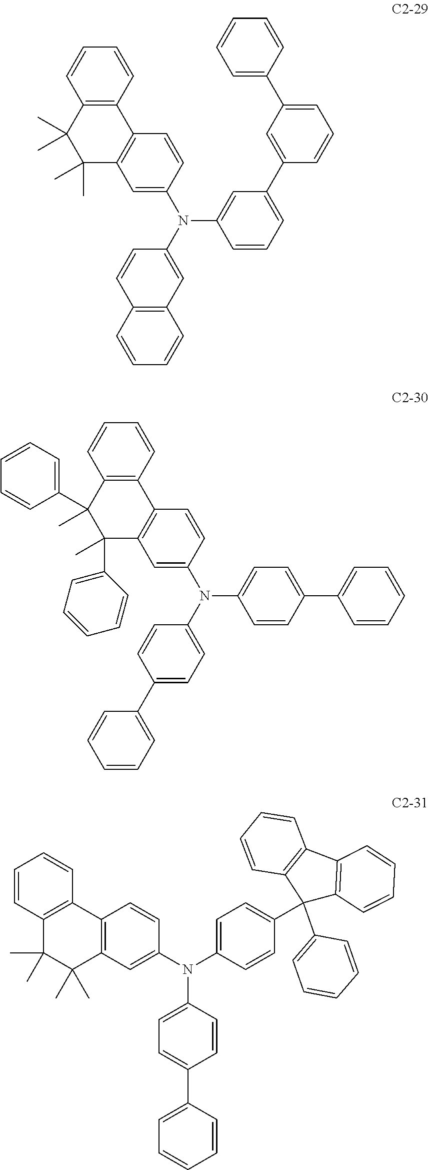 [SMILES: CC1(C)c2ccccc2-c2ccc(N(c3cccc(-c4cccc(-c5ccccc5)c4)c3)c3ccc4ccccc4c3)cc2C1(C)C ; CC1(c2ccccc2)c2ccccc2-c2ccc(N(c3ccc(-c4ccccc4)cc3)c3ccc(-c4ccccc4)cc3)cc2C1(C)c1ccccc1 ; CC1(C)c2ccccc2-c2ccc(N(c3ccc(-c4ccccc4)cc3)c3ccc(C4(c5ccccc5)c5ccccc5-c5ccccc54)cc3)cc2C1(C)C] 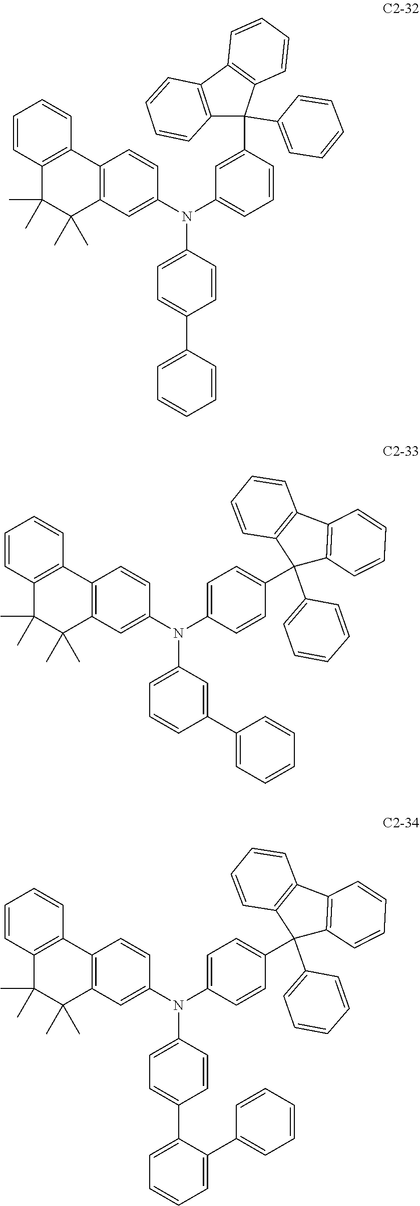 [SMILES: CC1(C)c2ccccc2-c2ccc(N(c3ccc(-c4ccccc4)cc3)c3cccc(C4(c5ccccc5)c5ccccc5-c5ccccc54)c3)cc2C1(C)C ; CC1(C)c2ccccc2-c2ccc(N(c3ccc(C4(c5ccccc5)c5ccccc5-c5ccccc54)cc3)c3cccc(-c4ccccc4)c3)cc2C1(C)C ; CC1(C)c2ccccc2-c2ccc(N(c3ccc(-c4ccccc4-c4ccccc4)cc3)c3ccc(C4(c5ccccc5)c5ccccc5-c5ccccc54)cc3)cc2C1(C)C] 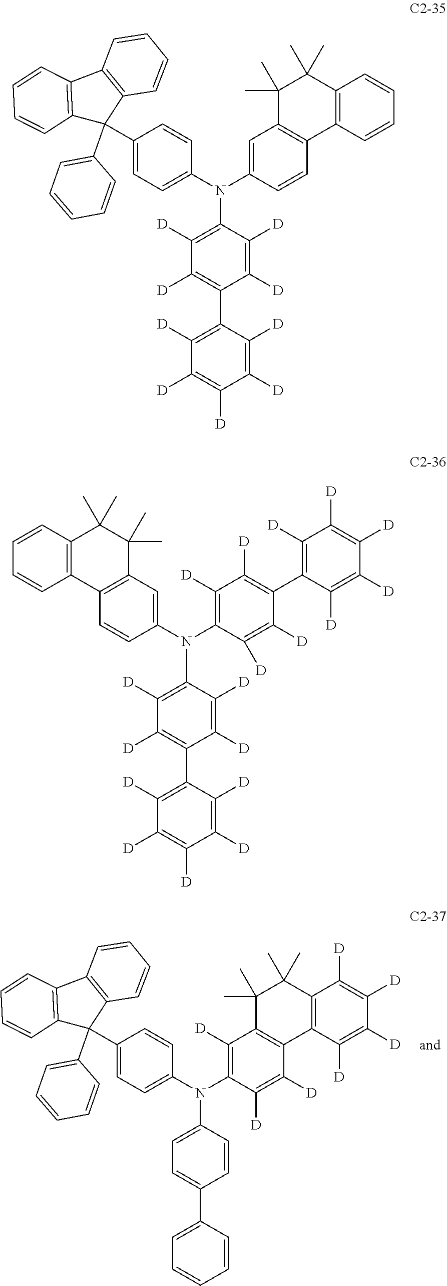 [SMILES: [2H]c1c([2H])c([2H])c(-c2c([2H])c([2H])c(N(c3ccc(C4(c5ccccc5)c5ccccc5-c5ccccc54)cc3)c3ccc4c(c3)C(C)(C)C(C)(C)c3ccccc3-4)c([2H])c2[2H])c([2H])c1[2H] ; [2H]c1c([2H])c([2H])c(-c2c([2H])c([2H])c(N(c3ccc4c(c3)C(C)(C)C(C)(C)c3ccccc3-4)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c2c(c1[2H])-c1c([2H])c([2H])c(N(c3ccc(-c4ccccc4)cc3)c3ccc(C4(c5ccccc5)c5ccccc5-c5ccccc54)cc3)c([2H])c1C(C)(C)C2(C)C] [chemical structure]
